(19) United States
(12) Patent Application Publication
(10) Pub. No.: US 2009/0305900 A1
(43) Pub. Date: Dec. 10, 2009

(54) GENEMAP OF THE HUMAN GENES ASSOCIATED WITH LONGEVITY

(76) Inventors: Abdelmajid Belouchi, Ville St-Laurent (CA); John Verner Raelson, Hudson (CA); Walter Edward Bradley, Montreal (CA); Bruno Paquin, Chateaugny (CA); Quynh Nguyen-Huu, Longvevil (CA); Pascal Croteau, Laval (CA); Rene Allard, Montreal (CA); Johanne Cousineau, Beaconsfield (CA); Nouzha Paquin, Chateaugny (CA); Paul Van Eerdewegh, Carlisle, MA (US); Randall David Little, Laval (CA); Tim Keith, Bedford, MA (US); Jonathan Segal, Efrat (IL)

Correspondence Address: DOWELL & DOWELL P.C., 103 Oronoco St., Suite 220, Alexandria, VA, 22314, US

(21) Appl. No.: 11/917,896

(22) PCT Filed: Jun. 19, 2006
(86) PCT No.: PCT/US06/23724

§ 371 Date: May 11, 2009

Related U.S. Application Data
(60) Provisional application No. 60/691,309, filed on Jun. 17, 2005.

(51) Int. Cl. C40B 30/00; C12Q 1/68; C07H 21/00; C40B 40/06; C12N 5/00; C12Q 1/02; G01N 33/68; G01N 27/26

(52) U.S. Cl. 506/7; 435/6; 536/23.1; 536/24.3; 506/16; 435/375; 435/29; 436/86; 204/450

(57) ABSTRACT

The present invention relates to the selection of a set of SNP markers for use in genome wide association studies based on linkage disequilibrium mapping. In particular, the invention relates to the fields of pharmacogenomics, diagnostics, patient therapy and the use of genetic haplotype information to predict an individual's longevity, their protection against age-related diseases and/or their response to a particular drug or drugs.

### PRIORITY

The present application claims priority from U.S. provisional application No. 60/691,309, Attorney Docket No. GENI-007/00US, filed Jun. 17, 2005, entitled GENEMAP OF THE HUMAN GENES ASSOCIATED WITH LONGEVITY, which is incorporated herein by reference.

The contents of the submission on compact discs are incorporated herein by reference in their entirety: A compact disc copy of the computer readable format copy of the Sequence Listing (HOME COPY) (filename: GENI00701WO SeqList, date recorded: Jun. 19, 2006, file size 14,313,000 bytes); a duplicate compact disc copy of the computer readable format copy of the Sequence Listing (SEARCH COPY) (filename: GENI00701WO SeqList, date recorded: Jun. 19, 2006, file size 14,313,000 bytes); and a triplicate copy of the computer readable format copy of the Sequence Listing (RECORD COPY) (filename: GENI00701WO SeqList, date recorded: Jun. 19, 2006, file size 14,313,000 bytes).

### FIELD OF THE INVENTION

The invention relates to the field of genomics and genetics, including genome analysis and the study of DNA variations. In particular, the invention relates to the fields of pharmacogenomics, diagnostics, patient therapy and the use of genetic haplotype information to predict an individual's longevity, their protection against age-related diseases and/or their response to a particular drug or drugs, so that drugs tailored to genetic differences of population groups may be developed and/or administered to the appropriate population.

The invention also relates to a GeneMap for longevity, which links variations in DNA (including both genic and non-genic regions) to an individual's longevity and susceptibility to age-related diseases and/or response to a particular drug or drugs. The invention further relates to the genes disclosed in the GeneMap (see Tables 4, 5 and 6), which are related to methods and reagents for detection of an individual's increased or decreased risk for aging related diseases by identifying at least one polymorphism in one or a combination of the genes from the GeneMap. Also related are the candidate regions identified in Table 1, which are associated with longevity. In addition, the invention further relates to nucleotide sequences of those genes including genomic DNA sequences, cDNA sequences, single nucleotide polymorphisms (SNPs), alleles and haplotypes (see Sequence Listing and Table 2, 3 and 7).

The present invention relates to the use of genes from Tables 4, 5 and 6, for determining an individual's likelihood of longevity, of being protected against cardiovascular related diseases (e.g., hypertension, diabetes mellitus, myocardial infarction, stroke, and/or transient ischemic attack), metabolic syndrome and/or other age-related diseases, and of retaining cognitive function with aging.

The invention further relates to isolated nucleic acids comprising these nucleotide sequences and isolated polypeptides or peptides encoded thereby. Also related, are expression vectors and host cells comprising the disclosed nucleic acids or fragments thereof, as well as antibodies that bind to the encoded polypeptides or peptides.

The present invention further relates to ligands that modulate the activity of the disclosed genes or gene products. In addition, the invention relates to diagnostics and therapeutics for aging related diseases, utilizing the disclosed nucleic acids, SNPs, chromosomal regions, gene maps, polypeptides or peptides, antibodies and/or ligands and small molecules that activate or repress relevant signaling events.

### BACKGROUND OF THE INVENTION

Many studies have documented the inheritance of human longevity (McGue et al., 1993); Ljungquist et al., 1998). Studies have also documented that centenarians (individuals who live for 100 years or more) are more likely than non-centenarians to have siblings who are long-lived. In particular, one study has shown that the siblings of centenarians have an approximately four-fold greater probability of survival to age 91 than siblings of non-centenarians (Perls et al., 1998). In addition, individuals who achieve exceptional longevity, such as centenarians, tend to live the majority of their lives in excellent health, demonstrating a rapid decline only at the end of their lives (Hitt et al., 1999). Genetic studies in other species including mammals indicate that specific genetic polymorphisms have powerful influences upon life span (defined by the age of the oldest member of the species). A number of studies on non-human species indicate that a relatively few genetic polymorphisms have a powerful influence upon the ability to achieve exceptional longevity. Many of those polymorphisms appear to play roles in basic mechanisms of metabolism and aging.

The DNA sequences between two human genomes are 99.9% identical. The variations in DNA sequence between individuals can be, for example, deletions of small or large stretches of DNA, insertions of stretches of DNA, variations in the number of repetitive DNA elements in non-coding regions, or changes in single base positions in the genome called “single nucleotide polymorphisms” (SNPs). Human DNA sequence variations account for a large fraction of observed differences between individuals, including susceptibility to disorders or a genetic link to traits, such as exceptional longevity.

Complex traits such as longevity are believed to involve several genes rather than single genes, as observed in rare traits. This makes detection of any particular gene substantially more difficult than in a rare trait, where a single gene mutation segregating according to a Mendelian inheritance pattern is the causative mutation. Any one of the multiple interacting gene mutations involved in the etiology of a complex and common trait will impart a lower relative risk for the trait than will the single gene mutation involved in a simple genetic trait. Low relative risk alleles are more difficult to detect and, as a result, the success of positional cloning using linkage mapping that was achieved for simple genetic trait genes has not been repeated for complex traits.

Several approaches have been proposed to discover and characterize multiple genes in complex genetic traits. These gene discovery methods can be subdivided into hypothesis-free disorder association studies and hypothesis-driven candidate gene or region studies. The candidate gene approach relies on the analysis of a gene in patients who have a disorder or a genetic trait in which the gene is thought to play a role. This approach is limited in utility because it only provides for the investigation of genes with known functions. Although variant sequences of candidate genes may be identified using this approach, it is inherently limited by the fact that variant sequences in other genes that contribute to the phenotype will be necessarily missed when the technique is employed. A genome-wide scan (GWS) has been shown to be efficient in identifying longevity susceptibility markers, such as the APOE gene on chromosome 19 and APOB gene on chromosome 2. In contrast to the candidate gene approach, a GWS searches throughout the genome without any a priori hypothesis and consequently can identify genes that are not obvious candidates for the complex genetic trait as well as genes that are relevant candidates for the trait. Furthermore, it can identify structurally important chromosomal regions that can influence the expression of specific, trait-related genes.

Family-based linkage mapping methods were initially used for disorder locus identification. This technique locates genes based on the relatively limited number of genetic recombination events within the families used in the study, and results in large chromosomal regions containing hundreds of genes, any one of which could be the trait-causing gene. Population-based, or linkage disequilibrium (LD) mapping is based on the premise that regions adjacent to a gene of interest are co-transmitted through the generations along with the gene. As a result, LD extends over shorter genetic regions than does linkage (Hewett et al., 2002), and can facilitate detection of genes with lower relative risk than family linkage mapping approaches. It also defines much smaller candidate regions which may contain only a few genes, making the identification of the actual trait gene much easier.

It has been estimated that a GWS that uses a general population and case/control association (LD) analysis would require approximately 700,000 SNP markers (Carlson et al., 2003). The cost of a GWS at this marker density for a sufficient sample size for statistical power is economically prohibitive. The use of a founder population (genetic isolates), such as the French Canadian population of Quebec, is one solution to the problem with LD analysis. The French Canadian population in Quebec (Quebec Founder Population—QFP) provides one of the best resources in the world for gene discovery based on its high levels of genetic sharing and genetic homogeneity. By combining DNA collected from the QFP, high throughput genotyping capabilities and proprietary algorithms for genetic analysis, a comprehensive genome-wide association study is facilitated. The present invention relates specifically to a set of longevity-related genes (GeneMap) and targets which present attractive points of therapeutic intervention for aging-associated diseases.

In view of the foregoing, identifying susceptibility genes associated with longevity and elucidating their respective biochemical pathways will facilitate the development of effective treatments for aging-associated diseases. This will also lead to the identification of diagnostic markers, which will predict the propensity for any such disease and allow therapeutic intervention before such disease occurs. The identification of genetic markers associated with longevity will lead to the development of effective therapeutic interventions for a much greater proportion of the individuals affected by aging-associated diseases. Knowledge of longevity-associated polymorphisms not only provides the benefit of predicting individual longevity, but also provides the ability to predict the likelihood of aging-associated diseases. The present invention satisfies this need and provides related advantages as well.

### BRIEF DESCRIPTION OF THE DRAWINGS

FIG. 1. Method employed by the inventors to permit the identification of genes predisposing to a particular genetic trait, such as longevity. The method can be applied for any given trait and the end result is the construction of a GeneMap for a particular trait or disorder. Briefly, a genetically heritable disorder or trait is selected followed by the preparation of an in-depth literature review on the prevalence, phenotypes, and available treatments (if relevant) of that trait. The literature review includes a list and description of candidate genes and regions associated with the trait. A clinical specialist in the field of the genetic trait is consulted for the definition of phenotype. Inclusion and exclusion criteria are then set and a study protocol is prepared. IRB and ethical approval are sought prior to patient recruitment. A network of physicians is required to recruit the necessary cases and controls for the study from the Quebec Founder Population. Individuals (cases and controls) are then recruited and DNA extraction and dosage is performed from the blood samples obtained. Samples are pooled into several cases and control pools. A GWS is performed on the pooled case and control samples using, as a minimum, the marker density determined from a study of linkage disequilibrium in the Quebec Founder Population; a study that led to the formulation of the Quebec linkage disequilibrium map (QLDM, a proprietary map of Genizon Biosciences Inc.). The results from the GWS genotyping are analyzed and candidate regions are selected for confirmatory mapping followed by fine mapping at a higher marker density in individual case and control samples in order to validate and/or refine the signal. The gene content of the candidate regions is analyzed and characterized. The representative haplotypes are then selected and sequenced. Once polymorphisms are identified by sequencing efforts the frequencies of genotypes and haplotypes in individual cases and controls are analyzed in a similar manner as for the GWS and fine mapping data. Ultrafine mapping is performed on all the samples to identify the polymorphisms that are most associated with the trait phenotype as part of the search for the actual DNA polymorphisms that confer susceptibility to the trait. The genes found associated with the trait, are then corroborated in a different population. The corroborated genes are used for the construction of a GeneMap.

### DESCRIPTION OF THE FILES CONTAINED ON THE CD-R

The contents of the submission on compact discs are incorporated herein by reference in their entirety: A compact disc copy of the computer readable format copy of the Sequence Listing (HOME COPY) (filename: GENI00701WO SeqList, date recorded: Jun. 19, 2006, file size 14,313,000 bytes); a duplicate compact disc copy of the computer readable format copy of the Sequence Listing (SEARCH COPY) (filename: GENI00701WO SeqList, date recorded: Jun. 19, 2006, file size 14,313,000 bytes); and a triplicate copy of the computer readable format copy of the Sequence Listing (RECORD COPY) (filename: GENI00701WO SeqList, date recorded: Jun. 19, 2006, file size 14,313,000 bytes).

The CD-R labeled “GeneMap of the Human Gene Associated with Longevity” contains the following one file of sequence listing. Each electronic copy of the sequence listing was created on Jun. 19, 2006 with a file size of 14,313 KB. The file name is as follows: GENI00701WO SeqList

### DEFINITIONS

Throughout the description of the present invention, several terms are used that are specific to the science of this field. For the sake of clarity and to avoid any misunderstanding, these definitions are provided to aid in the understanding of the specification and claims:

Allele: One of a pair, or series, of forms of a gene or non-genic region that occur at a given locus in a chromosome. Alleles are symbolized with the same basic symbol (e.g., B for dominant and b for recessive; B1, B2, Bn for n additive alleles at a locus). In a normal diploid cell there are two alleles of any one gene (one from each parent), which occupy the same relative position (locus) on homologous chromosomes. Within a population there may be more than two alleles of a gene. See multiple alleles. SNPs also have alleles, i.e., the two (or more) nucleotides that characterize the SNP

Amplification of nucleic acids: refers to methods such as polymerase chain reaction (PCR), ligation amplification (or ligase chain reaction, LCR) and amplification methods based on the use of Q-beta replicase. These methods are well known in the art and are described, for example, in U.S. Pat. Nos. 4,683,195 and 4,683,202. Reagents and hardware for conducting PCR are commercially available. Primers useful for amplifying sequences from the trait region, are preferably complementary to, and preferably hybridize specifically to, sequences in the trait region or in regions that flank a target region therein. Genes from Tables 4, 5 and 6 generated by amplification may be sequenced directly. Alternatively, the amplified sequence(s) may be cloned prior to sequence analysis.

Antigenic component: is a moiety that binds to its specific antibody with sufficiently high affinity to form a detectable antigen-antibody complex.

Antibodies: refer to polyclonal and/or monoclonal antibodies and fragments thereof, and immunologic binding equivalents thereof, that can bind to proteins and fragments thereof or to nucleic acid sequences from the trait region, particularly from the trait gene products or a portion thereof. The term antibody is used both to refer to a homogeneous molecular entity, or a mixture such as a serum product made up of a plurality of different molecular entities. Proteins may be prepared synthetically in a protein synthesizer and coupled to a carrier molecule and injected over several months into rabbits. Rabbit sera are tested for immunoreactivity to the protein or fragment. Monoclonal antibodies may be made by injecting mice with the proteins, or fragments thereof. Monoclonal antibodies will be screened by ELISA and tested for specific immunoreactivity with protein or fragments thereof (Harlow et al. 1988, Antibodies: A Laboratory Manual, Cold Spring Harbor Laboratory, Cold Spring Harbor, N.Y.). These antibodies will be useful in assays as well as therapeutics.

Associated allele: refers to an allele at a polymorphic locus that is associated with a particular phenotype of interest, e.g., a predisposition to a trait (e.g., longevity) or a particular drug response.

cDNA: refers to complementary or copy DNA produced from an RNA template by the action of RNA-dependent DNA polymerase (reverse transcriptase). Thus, a cDNA clone is a duplex DNA sequence complementary to an RNA molecule of interest, included in a cloning vector or amplified by PCR. This term includes genes from which the intervening sequences have been removed.

cDNA library: refers to a collection of recombinant DNA molecules containing cDNA inserts that together comprise essentially all of the expressed genes of an organism or tissue. A cDNA library can be prepared by methods known to one skilled in the art (see, e.g., Cowell and Austin, 1997, “DNA Library Protocols,” Methods in Molecular Biology). Generally, RNA is first isolated from the cells of the desired organism, and the RNA is used to prepare cDNA molecules.

Cloning: refers to the use of recombinant DNA techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.

Cloning vector: refers to a plasmid or phage DNA or other DNA molecule that is able to replicate in a host cell. The cloning vector is typically characterized by one or more endonuclease recognition sites at which such DNA sequences may be cleaved in a determinable fashion without loss of an essential biological function of the DNA, and which may contain a selectable marker suitable for use in the identification of cells containing the vector.

Coding sequence or a protein-coding sequence: is a polynucleotide sequence capable of being transcribed into mRNA and/or capable of being translated into a polypeptide or peptide. The boundaries of the coding sequence are typically determined by a translation start codon at the 5′-terminus and a translation stop codon at the 3′-terminus.

Complement of a nucleic acid sequence: refers to the antisense sequence that participates in Watson-Crick base-pairing with the original sequence.

Trait region (can also be referred to as a disorder region, such as age-related disorder for the longevity trait): refers to the portions of the human chromosomes displayed in Table 1 bounded by the markers from Table 1.

Trait-associated nucleic acid or polypeptide sequence: refers to a nucleic acid sequence that maps to a region of Table 1 or the polypeptides encoded therein (Table 2, 3 and 7 SNPs, nucleic acids, and polypeptides). For nucleic acids, this encompasses sequences that are identical or complementary to the gene sequences from Tables 4, 5 and 6, as well as sequence-conservative, function-conservative, and non-conservative variants thereof. For polypeptides, this encompasses sequences that are identical to the polypeptide, as well as function-conservative and non-conservative variants thereof. Included are the alleles of naturally-occurring polymorphisms causative of longevity such as, but not limited to, alleles that cause altered expression of genes of Tables 4, 5 and 6 and alleles that cause altered protein levels or stability (e.g., decreased levels, increased levels, expression in an inappropriate tissue type, increased stability, and decreased stability).

Expression vector: refers to a vehicle or plasmid that is capable of expressing a gene that has been cloned into it, after transformation or integration in a host cell. The cloned gene is usually placed under the control of (i.e., operably linked to) a regulatory sequence.

Function-conservative variants: are those in which a change in one or more nucleotides in a given codon position results in a polypeptide sequence in which a given amino acid residue in the polypeptide has been replaced by a conservative amino acid substitution. Function-conservative variants also include analogs of a given polypeptide and any polypeptides that have the ability to elicit antibodies specific to a designated polypeptide.

Founder population: also called a population isolate, this is a large number of people who have mostly descended, in genetic isolation from other populations, from a much smaller number of people who lived many generations ago.

Gene: refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions, as well as regulatory regions, and can include 5′ and 3′ ends. A gene sequence is wild-type if such sequence is usually found in individuals unaffected by the trait or condition of interest, e.g., longevity. However, environmental factors and other genes can also play an important role in the ultimate determination of the genetic trait. In the context of complex traits involving multiple genes (oligogenic traits), the wild type, or normal sequence can also be associated with a measurable risk or susceptibility, receiving its reference status based on its frequency in the general population.

GeneMaps are defined as groups of gene(s) that are directly or indirectly involved in at least one phenotype of a trait, e.g., longevity. As such, GeneMaps enable the development of synergistic diagnostic products, creating “theranostics”.

Genotype: Set of alleles at a specified locus or loci.

Haplotype: The allelic pattern of a group of (usually contiguous) DNA markers or other polymorphic loci along an individual chromosome or double helical DNA segment. Haplotypes identify individual chromosomes or chromosome segments. The presence of shared haplotype patterns among a group of individuals implies that the locus defined by the haplotype has been inherited, identical by descent (IBD), from a common ancestor. Detection of identical by descent haplotypes is the basis of linkage disequilibrium (LD) mapping. Haplotypes are broken down through the generations by recombination and mutation. In some instances, a specific allele or haplotype may be associated with susceptibility to a trait or condition of interest, e.g., longevity. In other instances, an allele or haplotype may be associated with a decrease in susceptibility to a trait or condition of interest, i.e., a protective sequence (see Table 7 for the significant haplotypes associated with longevity).

Host: includes prokaryotes and eukaryotes. The term includes an organism or cell that is the recipient of an expression vector (e.g., autonomously replicating or integrating vector).

Hybridizable: nucleic acids are hybridizable to each other when at least one strand of the nucleic acid can anneal to another nucleic acid strand under defined stringency conditions. In some embodiments, hybridization requires that the two nucleic acids contain at least 10 substantially complementary nucleotides; depending on the stringency of hybridization, however, mismatches may be tolerated. The appropriate stringency for hybridizing nucleic acids depends on the length of the nucleic acids and the degree of complementarity, and can be determined in accordance with the methods described herein.

Identity by descent (IBD): Identity among DNA sequences for different individuals that is due to the fact that they have all been inherited from a common ancestor. LD mapping identifies IBD haplotypes as the likely location of trait genes shared by a group of cases.

Identity: as known in the art, is a relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity and similarity can be readily calculated by known methods, including but not limited to those described in A. M. Lesk (ed), 1988, Computational Molecular Biology, Oxford University Press, NY; D. W. Smith (ed), 1993, Biocomputing. Informatics and Genome Projects, Academic Press, NY; A. M. Griffin and H. G. Griffin, H. G (eds), 1994, Computer Analysis of Sequence Data, Part 1, Humana Press, NJ; G. von Heinje, 1987, Sequence Analysis in Molecular Biology, Academic Press; and M. Gribskov and J. Devereux (eds), 1991, Sequence Analysis Primer, M Stockton Press, NY; H. Carillo and D. Lipman, 1988, SIAM J. Applied Math., 48:1073.

Immunogenic component: is a moiety that is capable of eliciting a humoral and/or cellular immune response in a host animal.

Isolated nucleic acids: are nucleic acids separated away from other components (e.g., DNA, RNA, and protein) with which they are associated (e.g., as obtained from cells, chemical synthesis systems, or phage or nucleic acid libraries). Isolated nucleic acids are at least 60% free, preferably 75% free, and most preferably 90% free from other associated components. In accordance with the present invention, isolated nucleic acids can be obtained by methods described herein, or other established methods, including isolation from natural sources (e.g., cells, tissues, or organs), chemical synthesis, recombinant methods, combinations of recombinant and chemical methods, and library screening methods.

Isolated polypeptides or peptides: are those that are separated from other components (e.g., DNA, RNA, and other polypeptides or peptides) with which they are associated (e.g., as obtained from cells, translation systems, or chemical synthesis systems). In a preferred embodiment, isolated polypeptides or peptides are at least 10% pure; more preferably, 80% or 90% pure. Isolated polypeptides and peptides include those obtained by methods described herein or other established methods, including isolation from natural sources (e.g., cells, tissues, or organs), chemical synthesis, recombinant methods, or combinations of recombinant and chemical methods. Proteins or polypeptides referred to herein as recombinant are proteins or polypeptides produced by the expression of recombinant nucleic acids. A portion as used herein with regard to a protein or polypeptide refers to fragments of that protein or polypeptide. The fragments can range in size from 5 amino acid residues to all but one residue of the entire protein sequence. Thus, a portion or fragment can be at least 5, 5-50, 50-100, 100-200, 200-400, 400-800, or more consecutive amino acid residues of a protein or polypeptide.

Linkage disequilibrium (LD): a statistical association between particular alleles at separate but linked loci, normally the result of a particular ancestral haplotype being common in the population studied. LD can also be defined as the situation in which the alleles for two or more loci do not occur together in individuals sampled from a population at frequencies predicted by the product of their individual allele frequencies. In other words, markers that are in LD do not follow Mendel's second law of independent random segregation. LD can be caused by any of several demographic or population artefacts as well as by the presence of genetic linkage between markers. However, when these artefacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait. The physical proximity of markers can be measured in family studies where it is called linkage or in population studies where it is called linkage disequilibrium.

LD mapping: population based gene mapping, which locates trait genes by identifying regions of the genome where haplotypes or marker variation patterns are shared statistically more frequently among cases compared to healthy controls. This method is based upon the assumption that many of the cases will have inherited an allele associated with the trait from a common ancestor (IBD), and that this allele will be in LD with the trait gene.

Locus: a specific position along a chromosome or DNA sequence. Depending upon context, a locus could be a gene, a marker, a chromosomal band or a specific sequence of one or more nucleotides.

Minor allele frequency (MAF): the population frequency of one of the alleles for a given polymorphism, which is equal or less than 50%. The sum of the MAF and the Major allele frequency equals one.

Markers: an identifiable DNA sequence that is variable (polymorphic) for different individuals within a population. These sequences facilitate the study of inheritance of a trait or a gene. Such markers are used in mapping the order of genes along chromosomes and in following the inheritance of particular genes; genes closely linked to the marker or in LD with the marker will generally be inherited with it. Two types of markers are commonly used in genetic analysis, microsatellites and SNPs.

Microsatellite: DNA of eukaryotic cells comprising a repetitive, short sequence of DNA that is present as tandem repeats and in highly variable copy number, flanked by sequences unique to that locus.

Mutant sequence: a sequence that differs from one or more wild-type sequences. For example, a nucleic acid from a gene listed in Tables 4, 5 and 6 containing a particular allele of a single nucleotide polymorphism may be a mutant sequence. In some cases, the individual carrying this allele has increased susceptibility toward the trait, or condition of interest. In other cases, the mutant sequence might also refer to an allele that decreases the susceptibility toward a trait or condition of interest and thus acts in a protective manner. The term mutation may also be used to describe a specific allele at a polymorphic locus.

Non-conservative variants: are those in which a change in one or more nucleotides in a given codon position results in a polypeptide sequence in which a given amino acid residue in a polypeptide has been replaced by a non-conservative amino acid substitution. Non-conservative variants also include polypeptides comprising non-conservative amino acid substitutions.

Nucleic acid or polynucleotide: purine- and pyrimidine-containing polymers of any length, either polyribonucleotides or polydeoxyribonucleotide or mixed polyribo polydeoxyribonucleotides. This includes single- and double-stranded molecules, i.e., DNA-DNA, DNA-RNA and RNA-RNA hybrids, as well as protein nucleic acids (PNA) formed by conjugating bases to an amino acid backbone. This also includes nucleic acids containing modified bases.

Nucleotide: consist of a ribose or deoxyribose sugar joined to a purine or pyrimidine base and to a phosphate group and that are the basic structural units of RNA and DNA. For its incorporation in DNA, nucleotides need to possess three phosphate esters but they are converted into monoesters in the process of incorporation.

Operably linked: means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

Ortholog: denotes a gene or polypeptide obtained from one species that has homology to an analogous gene or polypeptide from a different species.

Paralog: denotes a gene or polypeptide obtained from a given species that has homology to a distinct gene or polypeptide from that same species.

Phenotype: any visible, detectable or otherwise measurable property of an organism such as symptoms of, or susceptibility to, a disorder or trait.

Polymorphism: occurrence of two or more alternative genomic sequences or alleles between or among different genomes or individuals at a single locus. A polymorphic site thus refers specifically to the locus at which the variation occurs. In some cases, an individual carrying a particular allele of a polymorphism has an increased or decreased susceptibility toward a trait or condition of interest.

Portion and fragment: are synonymous. A portion as used with regard to a nucleic acid or polynucleotide refers to fragments of that nucleic acid or polynucleotide. The fragments can range in size from 8 nucleotides to all but one nucleotide of the entire gene sequence. Preferably, the fragments are at least about 8 to about 10 nucleotides in length; at least about 12 nucleotides in length; at least about 15 to about 20 nucleotides in length; at least about 25 nucleotides in length; or at least about 35 to about 55 nucleotides in length.

Probe or primer: refers to a nucleic acid or oligonucleotide that forms a hybrid structure with a sequence in a target region of a nucleic acid due to complementarity of the probe or primer sequence to at least one portion of the target region sequence.

Protein and polypeptide: are synonymous. Peptides are defined as fragments or portions of polypeptides, preferably fragments or portions having at least one functional activity (e.g., proteolysis, adhesion, fusion, antigenic, or intracellular activity) as the complete polypeptide sequence.

Recombinant nucleic acids: nuclei acids which have been produced by recombinant DNA methodology, including those nucleic acids that are generated by procedures which rely upon a method of artificial replication, such as the polymerase chain reaction (PCR) and/or cloning into a vector using restriction enzymes. Portions of recombinant nucleic acids which code for polypeptides can be identified and isolated by, for example, the method of M. Jasin et al., U.S. Pat. No. 4,952,501.

Regulatory sequence: refers to a nucleic acid sequence that controls or regulates expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Regulatory sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.

Sample: as used herein refers to a biological sample, such as, for example, tissue or fluid isolated from an individual or animal (including, without limitation, plasma, serum, cerebrospinal fluid, lymph, tears, nails, hair, saliva, milk, pus, and tissue exudates and secretions) or from in vitro cell culture-constituents, as well as samples obtained from, for example, a laboratory procedure.

Single nucleotide polymorphism (SNP): variation of a single nucleotide. This includes the replacement of one nucleotide by another and deletion or insertion of a single nucleotide. Typically, SNPs are biallelic markers although tri- and tetra-allelic markers also exist. For example, SNP A\C may comprise allele C or allele A (Table 2, 3 and 7). Thus, a nucleic acid molecule comprising SNP A\C may include a C or A at the polymorphic position. For a combination of SNPs, the term “haplotype” is used, e.g. the genotype of the SNPs in a single DNA strand that are linked to one another. In certain embodiments, the term “haplotype” is used to describe a combination of SNP alleles, e.g., the alleles of the SNPs found together on a single DNA molecule. In specific embodiments, the SNPs in a haplotype are in linkage disequilibrium with one another.

Sequence-conservative: variants are those in which a change of one or more nucleotides in a given codon position results in no alteration in the amino acid encoded at that position (i.e., silent mutation).

Substantially homologous: a nucleic acid or fragment thereof is substantially homologous to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases. Alternatively, substantial homology exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90% (M. Kanehisa, 1984, NucL Acids Res. 11:203-213). The length of homology comparison, as described, may be over longer stretches, and in certain embodiments will often be over a stretch of at least 14 nucleotides, usually at least 20 nucleotides, more usually at least 24 nucleotides, typically at least 28 nucleotides, more typically at least 32 nucleotides, and preferably at least 36 or more nucleotides.

Wild-type gene from Tables 4, 5 and 6: refers to the reference sequence. The wild-type gene sequences from Tables 4, 5 and 6 used to identify the variants (single nucleotide polymorphisms, alleles, and haplotypes) described in detail herein.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. Standard reference works setting forth the general principles of recombinant DNA technology include J. Sambrook et al., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC Press, Boca Raton; M. J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes I and 11; M. J. Gait (ed), 1984, Oligonucleotide Synthesis; B. D. Hames and S. J. Higgins (eds), 1984, Nucleic Acid Hybridization; Quirke and Taylor (eds), 1991, PCR-A Practical Approach; Harries and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning, J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology. Tools for Genomic and Molecular Research, American Society for Microbiology, Washington, D.C. Standard reference works setting forth the general principles of immunology include S. Sell, 1996, Immunology, Immunopathology & Immunity, 5th Ed., Appleton & Lange, Publ., Stamford, Conn.; D. Male et al., 1996, Advanced Immunology, 3d Ed., Times Mirror Int'l Publishers Ltd., Publ., London; D. P. Stites and A. L Terr, 1991, Basic and Clinical Immunology, 7th Ed., Appleton & Lange, Publ., Norwalk, Conn.; and A. K. Abbas et al., 1991, Cellular and Molecular Immunology, W. B. Saunders Co., Publ., Philadelphia, Pa. Any suitable materials and/or methods known to those of skill can be utilized in carrying out the present invention; however, preferred materials and/or methods are described. Materials, reagents, and the like to which reference are made in the following description and examples are generally obtainable from commercial sources, and specific vendors are cited herein.

### DETAILED DESCRIPTION OF THE INVENTION

Aging is a process in which all individuals of a species undergo a progressive decline in vitality leading to death. In metazoans, aging at the level of the whole organism is clearly evident. Aging of an organism represents the effects of entropy over time and has also been shown by many to be genetically programmed. While the effect of genetics on life expectancy is minimal across ages, this is not the case with centenarians (a rare phenotype achieved by 1 in 10,000 individuals). Siblings of current centenarians have odds ratios of between 8 and 17 of achieving 100 years of age, and parents of centenarians have an odds ratio of 7 for achieving ages 90-99 compared to appropriate controls. Furthermore, the offspring of long-lived parents have a significantly lower prevalence (50%) of hypertension, diabetes mellitus, myocardial infarctions and strokes/transient ischemic attacks compared with several age-matched control groups. In support of the inheritance of longevity, the New England Centenarian Study reported a statistically significant linkage between a genetic locus on chromosome 4 and exceptional longevity among siblings of centenarians.

Evidence in support of a genetic determinant for aging has been obtained in various organisms. For instance, in the yeast Saccharomyces cerevisiae, the patterns of expression of certain genes change in a specific manner during the life span, and these changed patterns suggest that the aging process is subject to gene regulation. Controlled expression of the transforming gene of Harvey murine sarcoma virus (v-Ha-ras) was found to extend yeast life span (as measured by the number of cell divisions) nearly two-fold (Jazwinski et al., 1993). RAS1 and RAS2, which are yeast homologs of the v-Ha-ras oncogene, play central roles in the integration of cell growth and the cell cycle in yeast. The primary role of these RAS proteins in yeast is the GTP-dependent regulation of adenylate cyclase activity. Curiously, mutations in RAS1 and RAS2 have opposite effects on yeast life span. The deletion of RAS1 lengthened life span while deletion of RAS2 decreased life span. D'mello, N. P. et al. (1994) isolated a yeast gene denoted longevity-assurance gene-1 (LAG1). LAG1 expression is highest in young cells and decreases as yeast cells age.

Furthermore, numerous diseases and disorders are associated with aging. Diseases which show age-dependent onset of symptoms include Alzheimer's disease, Pick's disease, Huntington's disease, Parkinson's disease, adult onset myotonic dystrophy, multiple sclerosis, adult onset leukodystrophy, diabetes mellitus, arteriosclerosis, and cancer.

Patients who suffer from premature aging syndromes exhibit numerous defects associated with more advanced age groups. Symptoms of Werner's syndrome include scleroderma-like skin changes, cataracts, subcutaneous calcification, premature arteriosclerosis, and diabetes mellitus. A striking aspect of Werner's syndrome, presumably arising from the same genetic defect, is a dramatic shortening of the replicative life-span of dermal fibroblasts in vitro (Faragher et al., 1993).

Scientists have also found that substantially reducing an organism's caloric intake increases longevity in mammals. Caloric restriction also known as “undernutrition without malnutrition” refers to a daily diet having about 30 to 40% fewer calories than the typical daily diet, but which contains the required nutrients and vitamins to support life. Caloric restriction extends both the maximal and the average life span of mice. In addition, preliminary studies suggest that calorie-restricted monkeys are healthier and tend to live longer than their freely fed counterparts (Mattison et al., 2003). In addition to increasing an organism's life span, caloric restriction plays a role in preventing or delaying many age-associated diseases and conditions, such as heart disease, dementia, and cancer. It has been found that caloric restriction not only slows the effects of aging on the nervous system, but studies suggest that it boosts the immune system and delays the onset of certain age-related cancers.

Mitochodria have also been implicated in age-related diseases. Mitochondria are cellular organelles often referred to as the “powerhouses” of the cell because they are the sites for cellular respiration or energy production in the cell. Indeed, mitochondria generate most of the energy of the cell primarily through oxidative phosphorylation, a complex process that uses electrons generated through oxidation of glucose and fatty acids to generate ATP. Aging mitochondria suffer from impaired function, which is associated with a variety of functional deficits (both physical and cognitive) and also the development of degenerative diseases. Proteins of the mitochondria oxidative phosphorylation complex have been shown to be impaired upon aging, which leads to a higher production of reactive oxygen species (ROS) and a decrease in efficiency of energy production. Free radicals produced by aerobic respiration cause cumulative oxidative damage resulting in aging and cell death. The biggest impact of age-related increase in ROS appears to be on somatic tissues composed of post-mitotic non-replicative cells including muscles, e.g., cardiac and skeletal, and nervous tissues, e.g., brain, retinal pigment epithelium. Numerous age-related changes have been reported in mitochondria. For example, oxidative damage to mitochondria DNA (mt DNA) increases with aging (Beckman et al., 1999) along with the oxidation of glutathione (GSH) a major intracellular antioxidant system, which plays an important role in protection against age-related mt DNA oxidative damage.

A relative lack of polymorphic variants associated with diseases of aging may be one prerequisite to achieving exceptional longevity. For example, the absence of genetic polymorphisms among centenarians is exemplified by the rarity of the apolipoprotein E E4 allele that has been associated with Alzheimer's disease and cardiovascular disease (Schachter et al., 1994). Another prerequisite to achieving exceptional longevity may be the ability to modulate the rate of the aging process, which also appears to have a genetic component. For example, one study has shown that the offspring of centenarians had more favorable lipid profile characteristics (Barzilai et al., 2001).

Thus, while there are a number of genetic studies and markers known, longevity and age-related diseases are not well understood. There is also a continuing need in the medical arts for genetic markers of longevity and guidance for the use of such markers. Compositions and methods of the invention are useful for predicting the propensity for exceptional longevity in humans. Additionally, compositions and methods of the invention are useful for predicting the propensity for age-related diseases including, but not limited to heart disease, cardiovascular disease, stroke, Alzheimer's disease, cancer, and ocular disease. Additionally, compositions and methods of the invention are useful for indicating possible early therapeutic intervention to prevent or to lessen the effects of diseases associated with aging. The present invention fulfills this need and provides further related advantages.

### Genome Wide Association Study to Construct a GeneMap for Longevity

The present invention is based on the discovery of genes associated with longevity. In the preferred embodiment, trait-associated loci (candidate regions; Tables 1-7) are therefore identified by the statistically significant differences in allele frequencies between the cases and the controls. For the purpose of the present invention, 47 candidate regions showing a difference with a −log 10 P value of 3.0 or higher are identified. The only previously replicated locus associated with longevity is at 4q24-q25 (Puca et al., 2001).

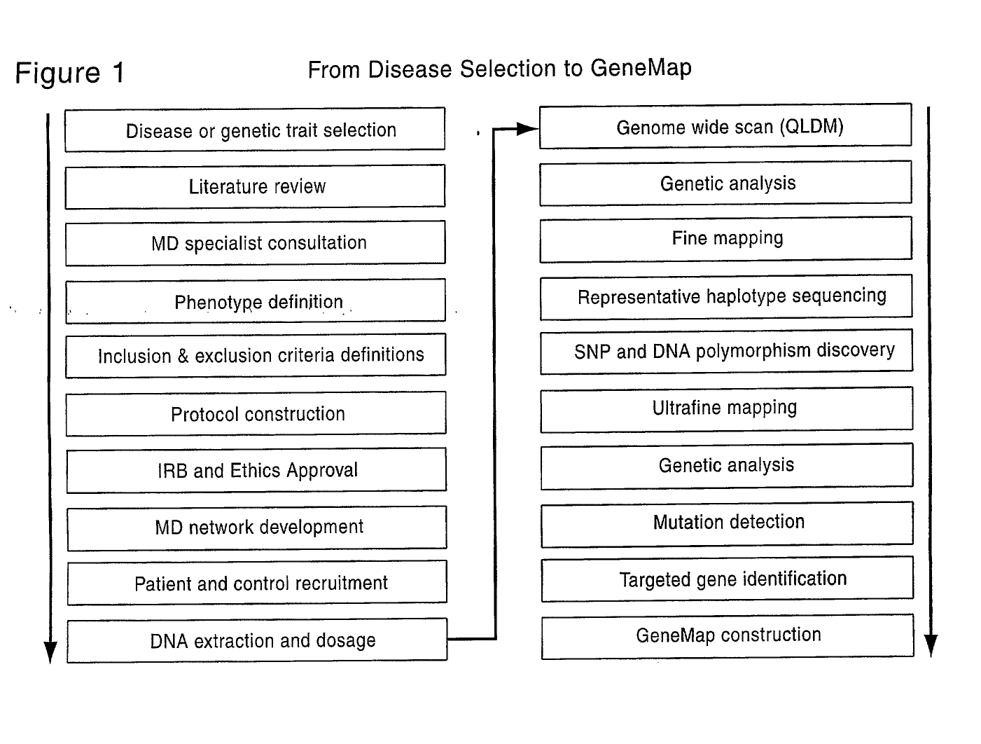

The invention provides a method for the discovery of genes associated with longevity and the construction of a GeneMap for longevity in a human population, comprising the following steps (see FIG. 1 and Example section herein):

### Step 1: Recruit Cases and Controls

In the preferred embodiment, 500 cases ascertained to be 94 years old or older along with 500 control individuals are recruited from the Quebec Founder Population (QFP).

In another embodiment, 615 cases ascertained to be 94 years old or older along with 615 control individuals, ascertained to be 65 years old or younger, are recruited from the Quebec Founder Population (QFP).

In another embodiment, the present invention is performed as a whole or partially with DNA samples from individuals of another founder population than the Quebec population or from the general population.

### Step 2: DNA Extraction and Dosage

Any sample comprising cells or nucleic acids from patients or controls may be used. Preferred samples are those easily obtained from the patient or control. Such samples include, but are not limited to blood, peripheral lymphocytes, buccal swabs, epithelial cell swabs, nails, hair, bronchoalveolar lavage fluid, sputum, or other body fluid or tissue obtained from an individual.

In the preferred embodiment, DNA is extracted from such samples in the quantity and quality necessary to perform the invention using conventional DNA extraction and dosage techniques. The present invention is not linked to any DNA extraction or dosage platform in particular.

The extracted DNA from case and control samples from recruited individuals is pooled together in various pools. Pools are designed to segregate cases from controls, and males from females.

In the preferred embodiment, proband pools consist of DNA extracted from recruited cases and control pools consist of DNA extracted from control individuals. The probands are also segregated according to their age at the time of recruitment and the proband females are further separated in two groups, those who failed a cognitive test and those who passed the test. Two proband male pools contain preferably 53-74 individuals, separated by age group. One proband female pool contains the 71 females who failed a cognitive test whereas the 7 remaining pools consist of 43-80 proband females who passed the test, separated by age group. Preferably, the ten (10) control pools consist of 8 pools of 61 female samples and 2 pools of 63-64 male samples.

In the preferred embodiment proband samples of 615 cases (127 males and 488 females) are used to construct the case pools and 615 controls (127 males and 488 females) are used to construct the control pools.

### Step 3: Genotype the Proband and Control Pools

In the preferred embodiment, assay specific and/or locus-specific and/or allele-specific oligonucleotides for every SNP marker of the present invention (Table 2) are organized onto one or more arrays. The genotype at each SNP locus is revealed by hybridizing short PCR fragments comprising each SNP locus onto these arrays. The arrays permit a high-throughput genome wide association study using DNA samples from individuals of the Quebec founder population. Such assay-specific and/or locus-specific and/or allele-specific oligonucleotides necessary for scoring each SNP of the present invention are preferably organized onto a solid support. Such supports can be arrayed on wafers, glass slides, beads or any other type of solid support.

In another embodiment, the assay-specific and/or locus-specific and/or allele-specific oligonucleotides are not organized onto a solid support but are still used as a whole, in panels or one by one. The present invention is therefore not linked to any genotyping platform in particular.

In another embodiment, one or more portions of the SNPs maps (publicly available maps, proprietary maps from Perlegen Sciences, Inc. (Mountain View, Calif., USA), and our own proprietary QLDM map) are used to screen the whole genome, a subset of chromosomes, a chromosome, a subset of genomic regions or a single genomic region.

Step 4: Exclude the Markers that Did not Pass the Quality Control of the Assay

Preferably, the quality controls consist of, but are not limited to, the following criteria: eliminate SNPs that are non-polymorphic in the Quebec founder population or have ≦10% minor allele frequency (MAF).

### Step 5: Perform the Genetic Analysis on the Results Obtained

In the preferred embodiment, genetic analysis is performed on all the genotypes from step 3.

In another embodiment, genetic analysis is performed on a total of 248,535 SNPs.

In one embodiment, the data analysis compares the relative fluorescence intensities of features corresponding to the reference allele of a given SNP with those corresponding to the alternate allele, to calculate a p-hat value. The latter is proportional to the fluorescence signal from perfect match features for the reference allele divided by the sum of fluorescence signals from perfect match features for the reference plus the alternate alleles. P-hat assumes values close to 1 (typically 0.9) for pure reference samples and close to 0 (typically 0.1) for pure alternate samples, and can be used as a measured estimate of the reference allele frequency of a SNP in a DNA pool. The difference between case and control pools, delta p-hat, is calculated using the weighted average of case and control p-hats. Delta p-hat is a reliable estimate of the allele frequency difference between the cases and controls.

In yet another embodiment, the data is analyzed according to the p-hat value obtained from the previous embodiment on each pool. Single marker P values are calculated for all markers within the genome wide scan map as described in Example 3 herein, using the p-hat value.

In another embodiment, the combined P values across multi-marker sliding windows are calculated after the method of Fisher (described in Example 3 herein).

### Step 6: Fine Mapping and Confirmatory Mapping

In this step, the candidate regions that were identified by step 5 are further mapped and confirmed for the purpose of refinement and validation.

In the preferred embodiment, the cases and controls are individually genotyped to confirm the candidate regions. The confirmed candidate regions are processed by fine mapping to refine the candidate regions.

In the preferred embodiment, this fine mapping is performed with a density of genetic markers higher than in the genome wide scan (step 3) using any genotyping platform available in the art. Such fine mapping can also be performed with fewer genetic markers than in the GWS. Such fine mapping can be, but is not limited to, typing the allele via an allele-specific elongation assay that is then ligated to a locus-specific oligonucleotide. Such assays can be performed directly on the genomic DNA at a highly multiplex level and the products can be amplified using universal oligonucleotides. For each candidate region, the density of genetic markers can be, but is not limited to, a set of SNP markers with an average inter-marker distance of 1-4 Kb distributed over about 400 Kb to 1 Mb, roughly centered at the highest point of the GWS association. The preferred samples are those obtained from longevity samples including the ones used for the GWS.

In the preferred embodiment, the genetic analysis of the results obtained using haplotype information (available after confirmatory mapping of individual samples, see Examples section herein) as well as single-marker association (as performed as in step 5, described herein) are performed as described herein (see Example section). The candidate regions that are validated and confirmed after this analysis proceed to a gene mining step described in Example 5, herein, to characterize their marker and genetic content.

### Step 7: SNP and DNA Polymorphism Discovery

In the preferred embodiment, all the candidate genes and regions identified in step 6 are sequenced for polymorphism identification.

In another embodiment, the entire region, including all introns, is sequenced to identify all polymorphisms.

In yet another embodiment, the candidate genes are prioritized for sequencing, and only functional gene elements (promoters, exons and splice sites) are sequenced.

In yet another embodiment, previously identified polymorphisms in the candidate regions can also be used. For example, SNPs from dbSNP, Perlegen Sciences, Inc., or others can also be used rather than resequencing the candidate regions to identify polymorphisms.

The discovery of SNPs and DNA polymorphisms generally comprises a step consisting of determining the major haplotypes in the region to be sequenced. The preferred samples are selected according to which haplotypes contribute to the association signal observed in the region to be sequenced. The purpose is to select a set of samples that covers all the major haplotypes in the given region. Each major haplotype is preferably analyzed in at least a few individuals.

Any analytical procedure may be used to detect the presence or absence of variant nucleotides at one or more polymorphic positions of the invention. In general, the detection of allelic variation requires a mutation discrimination technique, optionally an amplification reaction and optionally a signal generation system. Any means of mutation detection or discrimination may be used. For instance, DNA sequencing, scanning methods, hybridization, extension based methods, incorporation based methods, restriction enzyme-based methods and ligation-based methods may be used in the methods of the invention.

Sequencing methods include, but are not limited to, direct sequencing, and sequencing by hybridization. Scanning methods include, but are not limited to, protein truncation test (PTT), single-strand conformation polymorphism analysis (SSCP), denaturing gradient gel electrophoresis (DGGE), temperature gradient gel electrophoresis (TGGE), cleavage, heteroduplex analysis, chemical mismatch cleavage (CMC), and enzymatic mismatch cleavage. Hybridization-based methods of detection include, but are not limited to, solid phase hybridization such as dot blots, multiple allele specific diagnostic assay (MASDA), reverse dot blots, and oligonucleotide arrays (DNA Chips). Solution phase hybridization amplification methods may also be used, such as Taqman. Extension based methods include, but are not limited to, amplification refraction mutation systems (ARMS), amplification refractory mutation systems (ALEX), and competitive oligonucleotide priming systems (COPS). Incorporation based methods include, but are not limited to, mini-sequencing and arrayed primer extension (APEX). Restriction enzyme-based detection systems include, but are not limited to, restriction site generating PCR. Lastly, ligation based detection methods include, but are not limited to, oligonucleotide ligation assays (OLA). Signal generation or detection systems that may be used in the methods of the invention include, but are not limited to, fluorescence methods such as fluorescence resonance energy transfer (FRET), fluorescence quenching, fluorescence polarization as well as other chemiluminescence, electrochemiluminescence, Raman, radioactivity, colometric methods, hybridization protection assays and mass spectrometry methods. Further amplification methods include, but are not limited to self sustained replication (SSR), nucleic acid sequence based amplification (NASBA), ligase chain reaction (LCR), strand displacement amplification (SDA) and branched DNA (B-DNA).

### Step 8: Ultrafine Mapping

This step further maps the candidate regions and genes confirmed in the previous step to identify and validate the responsible polymorphisms associated with longevity in the human population.

In a preferred embodiment, the discovered SNPs and polymorphisms of step 7 are ultrafine mapped at a higher density of markers than the fine mapping described herein using the same technology described in step 6.

### Step 9: GeneMap Construction

The confirmed variations in DNA (including both genic and non-genic regions) are used to build a GeneMap for longevity disorder. The gene content of this GeneMap is described in more detail below. Such GeneMap can be used for other methods of the invention comprising the diagnostic methods described herein, the susceptibility to longevity, the response to a particular drug, the efficacy of a particular drug, the screening methods described herein and the treatment methods described herein.

As is evident to one of ordinary skill in the art, all of the above steps or the steps of FIG. 1 do not need to be performed, or performed in a given order to practice or use the SNPs, genomic regions, genes, proteins, etc. in the methods of the invention.

Genes from the GeneMap

In the preferred embodiment the GeneMap consists of genes and targets, in a variety of combinations, identified from the candidate regions listed in Table 1. In the preferred embodiment, all genes from Tables 4, 5 and 6 are present in the GeneMap.

### Nucleic Acid Sequences

The nucleic acid sequences of the present invention may be derived from a variety of sources including DNA, cDNA, synthetic DNA, synthetic RNA, derivatives, mimetics or combinations thereof. Such sequences may comprise genomic DNA, which may or may not include naturally occurring introns, genic regions, nongenic regions, and regulatory regions. Moreover, such genomic DNA may be obtained in association with promoter regions or poly (A) sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means. The nucleic acids described herein are used in certain embodiments of the methods of the present invention for production of RNA, proteins or polypeptides, through incorporation into cells, tissues, or organisms. In one embodiment, DNA containing all or part of the coding sequence for the genes described in Tables 4, 5 and 6, or the SNP markers described in Tables 2, 3 and 7, is incorporated into a vector for expression of the encoded polypeptide in suitable host cells. The invention also comprises the use of the nucleotide sequence of the nucleic acids of this invention to identify DNA probes for the genes described in Tables 4, 5 and 6 or the SNP markers described in Table 2, 3 or 7, PCR primers to amplify the genes described in Tables 4, 5 and 6 or the SNP markers described in Tables 2, 3 and 7, nucleotide polymorphisms in the genes described in Tables 4, 5 and 6, and regulatory elements of the genes described in Tables 4, 5 and 6. The nucleic acids of the present invention find use as primers and templates for the recombinant production of longevity-associated peptides or polypeptides, for chromosome and gene mapping, to provide antisense sequences, for tissue distribution studies, to locate and obtain full length genes, to identify and obtain homologous sequences (wild-type and mutants), and in diagnostic applications.

### Antisense Oligonucleotides

In a particular embodiment of the invention, an antisense nucleic acid or oligonucleotide is wholly or partially complementary to, and can hybridize with, a target nucleic acid (either DNA or RNA) having the sequence of SEQ ID NO:1, NO:3 or any SEQ ID from Tables 2-7. For example, an antisense nucleic acid or oligonucleotide comprising 16 nucleotides can be sufficient to inhibit expression of at least one gene from Tables 4, 5 and 6. Alternatively, an antisense nucleic acid or oligonucleotide can be complementary to 5′ or 3′ untranslated regions, or can overlap the translation initiation codon (5′ untranslated and translated regions) of at least one gene from Tables 4, 5 and 6, or its functional equivalent. In another embodiment, the antisense nucleic acid is wholly or partially complementary to, and can hybridize with, a target nucleic acid that encodes a polypeptide from a gene described in Tables 4, 5 and 6.

In addition, oligonucleotides can be constructed which will bind to duplex nucleic acid (i.e., DNA:DNA or DNA:RNA), to form a stable triple helix containing or triplex nucleic acid. Such triplex oligonucleotides can inhibit transcription and/or expression of a gene from Tables 4, 5 and 6, or its functional equivalent (M. D. Frank-Kamenetskii et al., 1995). Triplex oligonucleotides are constructed using the base-pairing rules of triple helix formation and the nucleotide sequence of the genes described in Tables 4, 5 and 6.

The present invention encompasses methods of using oligonucleotides in antisense inhibition of the function of the genes from Tables 4, 5 and 6. In the context of this invention, the term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention. Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some non-limiting examples of modifications at the 2′ position of sugar moieties which are useful in the present invention include OH, SH, SCH3, F, OCH3, OCN, O(CH2), NH2 and O(CH2)nCH3, where n is from 1 to about 10. Such oligonucleotides are functionally interchangeable with natural oligonucleotides or synthesized oligonucleotides, which have one or more differences from the natural structure. All such analogs are comprehended by this invention so long as they function effectively to hybridize with at least one gene from Tables 4, 5 and 6 DNA or RNA to inhibit the function thereof.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As defined herein, a “subunit” is a base and sugar combination suitably bound to adjacent subunits through phosphodiester or other bonds. Antisense nucleic acids or oligonucleotides can be produced by standard techniques (see, e.g., Shewmaker et al., U.S. Pat. No. 6,107,065). The oligonucleotides used in accordance with this invention may be conveniently and routinely made through the well-known technique of solid phase synthesis. Any other means for such synthesis may also be employed; however, the actual synthesis of the oligonucleotides is well within the abilities of the practitioner. It is also well known to prepare other oligonucleotide such as phosphorothioates and alkylated derivatives.

The oligonucleotides of this invention are designed to be hybridizable with RNA (e.g., mRNA) or DNA from genes described in Tables 4, 5 and 6. For example, an oligonucleotide (e.g., DNA oligonucleotide) that hybridizes to mRNA from a gene described in Tables 4, 5 and 6 can be used to target the mRNA for RnaseH digestion. Alternatively, an oligonucleotide that can hybridize to the translation initiation site of the mRNA of a gene described in Tables 4, 5 and 6 can be used to prevent translation of the mRNA. In another approach, oligonucleotides that bind to the double-stranded DNA of a gene from Tables 4, 5 and 6 can be administered. Such oligonucleotides can form a triplex construct and inhibit the transcription of the DNA encoding polypeptides of the genes described in Tables 4, 5 and 6. Triple helix pairing prevents the double helix from opening sufficiently to allow the binding of polymerases, transcription factors, or regulatory molecules. Recent therapeutic advances using triplex DNA have been described (see, e.g., J. E. Gee et al., 1994, Molecular and Immunologic Approaches, Futura Publishing Co., Mt. Kisco, N.Y.).

As non-limiting examples, antisense oligonucleotides may be targeted to hybridize to the following regions: mRNA cap region; translation initiation site; translational termination site; transcription initiation site; transcription termination site; polyadenylation signal; 3′ untranslated region; 5′ untranslated region; 5′coding region; mid coding region; and 3′ coding region. Preferably, the complementary oligonucleotide is designed to hybridize to the most unique 5′ sequence of a gene described in Tables 4, 5 and 6, including any of about 15-35 nucleotides spanning the 5′ coding sequence. In accordance with the present invention, the antisense oligonucleotide can be synthesized, formulated as a pharmaceutical composition, and administered to a subject. The synthesis and utilization of antisense and triplex oligonucleotides have been previously described (e.g., Simon et al., 1999; Barre et al., 2000; Elez et al., 2000; Sauter et al., 2000).

Alternatively, expression vectors derived from retroviruses, adenovirus, herpes or vaccinia viruses or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding a polypeptide from the genes described in Tables 4, 5 and 6. These techniques are described both in Sambrook et al., 1989 and in Ausubel et al., 1992. For example, expression of at least one gene from Tables 4, 5 and 6 can be inhibited by transforming a cell or tissue with an expression vector that expresses high levels of untranslatable sense or antisense sequences. Even in the absence of integration into the DNA, such vectors may continue to transcribe RNA molecules until they are disabled by endogenous nucleases. Transient expression may last for a month or more with a nonreplicating vector, and even longer if appropriate replication elements are included in the vector system. Various assays may be used to test the ability of gene-specific antisense oligonucleotides to inhibit the expression of at least one gene from Tables 4, 5 and 6. For example, mRNA levels of the genes described in Tables 4, 5 and 6 can be assessed by Northern blot analysis (Sambrook et al., 1989; Ausubel et al., 1992; J. C. Alwine et al. 1977; I. M. Bird, 1998), quantitative or semi-quantitative RT-PCR analysis (see, e.g., W. M. Freeman et al., 1999; Ren et al., 1998; J. M. Cale et al., 1998), or in situ hybridization (reviewed by A. K. Raap, 1998). Alternatively, antisense oligonucleotides may be assessed by measuring levels of the polypeptide from the genes described in Tables 4, 5 and 6, e.g., by western blot analysis, indirect immunofluorescence and immunoprecipitation techniques (see, e.g., J. M. Walker, 1998, Protein Protocols on CD-ROM, Humana Press, Totowa, N.J.). Any other means for such detection may also be employed, and is well within the abilities of the practitioner.

### Mapping Technologies

The present invention includes various methods which employ mapping technologies to map SNPs and polymorphisms. For purpose of clarity, this section comprises, but is not limited to, the description of mapping technologies that can be utilized to achieve the embodiments described herein. Mapping technologies may be based on amplification methods, restriction enzyme cleavage methods, hybridization methods, sequencing methods, and cleavage methods using agents.

Amplification methods include: self sustained sequence replication (Guatelli et al., 1990), transcriptional amplification system (Kwoh et al., 1989), Q-Beta Replicase (Lizardi et al., 1988), isothermal amplification (e.g. Dean et al., 2002; and Hafner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

Restriction enzyme cleavage methods include: isolating sample and control DNA, amplification (optional), digestion with one or more restriction endonucleases, determination of fragment length sizes by gel electrophoresis and comparing samples and controls. Differences in fragment length sizes between sample and control DNA indicates mutations in the sample DNA. Moreover, sequence specific ribozymes (see, e.g., U.S. Pat. No. 5,498,531) or DNAzyme (e.g. U.S. Pat. No. 5,807,718) can be used to score for the presence of specific mutations by development or loss of a ribozyme or DNAzyme cleavage site.

SNPs and SNP maps of the invention can be identified or generated by hybridizing sample nucleic acids, e.g., DNA or RNA, to high density arrays or bead arrays containing oligonucleotide probes corresponding to the SNPS of Tables 2, 3 and 7 (see the Affymetrix arrays and Illumina bead sets at www.affymetrix.com and www.illumina.com and see Cronin et al., 1996; or Kozal et al., 1996).

A variety of sequencing reactions known in the art can be used to directly sequence nucleic acids for the presence or the absence of one or more SNPs of Tables 2, 3 and 7. Examples of sequencing reactions include those based on techniques developed by Maxam and Gilbert (1977) or Sanger (1977). It is also contemplated that any of a variety of automated sequencing procedures can be utilized, including sequencing by mass spectrometry (see, e.g. PCT International Publication No. WO 94/16101; Cohen et al., 1996; and Griffin et al., 1993), real-time pyrophosphate sequencing method (Ronaghi et al., 1998; and Permutt et al., 2001) and sequencing by hybridization (see e.g. Drmanac et al., 2002).

Other methods of detecting SNPs include methods in which protection from cleavage agents is used to detect mismatched bases in RNA/RNA, DNA/DNA or RNA/DNA heteroduplexes (Myers et al., 1985). In general, the technique of “mismatch cleavage” starts by providing heteroduplexes formed by hybridizing (labeled) RNA or DNA containing a wild-type sequence with potentially mutant RNA or DNA obtained from a sample. The double-stranded duplexes are treated with an agent who cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of a mutation or SNP. (see, for example, Cotton et al., 1988; and Saleeba et al., 1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping SNPs. For example, the mutY enzyme of E. coli cleaves A at G/A mismatches (Hsu et al., 1994). Other examples include, but are not limited to, the MutHLS enzyme complex of E. coli (Smith and Modrich Proc. 1996) and Cel 1 from the celery (Kulinski et al., 2000) both cleave the DNA at various mismatches. According to an exemplary embodiment, a probe based on a polymorphic site corresponding to a SNP of Tables 2, 3 and 7 is hybridized to a cDNA or other DNA product from a test cell or cells. The duplex is treated with a DNA mismatch repair enzyme, and the cleavage products, if any, can be detected from electrophoresis protocols or the like. See, for example, U.S. Pat. No. 5,459,039. Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad et al., 2004).

In other embodiments, alterations in electrophoretic mobility can be used to identify SNPs in a sample. For example, single strand conformation polymorphism (SSCP) can be used to detect differences in electrophoretic mobility between mutant and wild type nucleic acids (Orita et al., 1989; Cotton et al., 1993; and Hayashi 1992). Single-stranded DNA fragments of case and control nucleic acids will be denatured and allowed to renature. The secondary structure of single-stranded nucleic acids varies according to sequence. The resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et al., 1985). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoorn et al., 2000).

Examples of other techniques for detecting SNPs include, but are not limited to, selective oligonucleotide hybridization, selective amplification, selective primer extension, selective ligation, single-base extension, selective termination of extension or invasive cleavage assay. For example, oligonucleotide primers may be prepared in which the SNP is placed centrally and then hybridized to target DNA under conditions which permit hybridization only if a perfect match is found (Saiki et al., 1986; Saiki et al., 1989). Such oligonucleotides are hybridized to PCR amplified target DNA or a number of different mutations when the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

Hybridization assays may also be carried out with a temperature gradient following the principle of dynamic allele-specific hybridization or like e.g. Jobs et al., (2003); and Bourgeois and Labuda, (2004). For example, the hybridization is done using one of the two allele-specific oligonucleotides labeled with a fluorescent dye, an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of an associated allele, a particular allele of a polymorphic locus, or the like (e.g. Jobs et al., 2003). Alternatively, the hybridization is done under a gradually decreasing temperature. In this case, both allele-specific oligonucleotides are hybridized to the template competitively. At high temperature none of the two probes are hybridized. Once the optimal temperature of the full-matched probe is reached, it hybridizes and leaves no target for the mismatched probe (e.g. Bourgeois and Labuda, 2004). In the latter case, if the allele-specific probes are differently labeled, then they are hybridized to a single PCR-amplified target. If the probes are labeled with the same dye, then the probe cocktail is hybridized to twice to identical templates with only one labeled probes, different in the two cocktails, in the presence of the unlabeled competitive probe.

Alternatively, allele specific amplification technology that depends on selective PCR amplification may be used in conjunction with the present invention. Oligonucleotides used as primers for specific amplification may carry the associated allele, a particular allele of a polymorphic locus, or the like, also referred to as “mutation” of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the mutation to create cleavage-based detection (Gasparini et al., 1992). It is anticipated that in certain embodiments, amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., 1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNPs and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78 2003).

Another alternative includes the single-base extension/ligation assay using a molecular inversion probe, consisting of a single, long oligonucleotide (see e.g. Hardenbol et al., 2003). In such an embodiment, the oligonucleotide hybridizes on both side of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, and bead array or like. This reaction can be performed at a high multiplexed level.

In another alternative, the associated allele, a particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, and bead array or like. In the latter case, the extension oligonucleotide will contain a SNP-specific zip code tag.

In yet another alternative, a SNP is scored by selective termination of extension. The template is first amplified by PCR and the extension oligonucleotide hybridizes in vicinity to the SNP locus, close to but not necessarily adjacent to it. The extension reaction is carried out using a thermostable polymerase such as Thermo Sequenase (GE Healthcare) in the presence of a mix of dNTPs and at least one ddNTP. The latter has to terminate the extension at one of the allele of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, SNPs are detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Methods to Identify Agents that Modulate the Expression of a Nucleic Acid Encoding a Gene Involved in Longevity.

The present invention provides methods for identifying agents that modulate the expression of a nucleic acid encoding a gene from Tables 4, 5 and 6. Such methods may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell. Such cells can be obtained from any parts of the body such as the scalp, blood, dermis, epidermis and other skin cells, cutaneous surfaces, intertrigious areas, genitalia, vessels and endothelium. Some non-limiting examples of cells that can be used are red blood cells, muscle cells, heart cells, nerve cells, insulin-producing cells, pancreatic cells, brain cells, germ cells, keratinocytes, monocytes, neutrophils, langerhans cells, CD4+ and CD8+ T cells, B and T lymphocytes, leukocytes, hormonal cells, bone marrow cells, skin cells, buccal cells, spinal cord cells, bone cells, adipose cells, cartilage cells, dendritic cells, intestinal cells, hepatic cells, mucous cells, olfactory cells, retinal cells, somatic cells and arterial cells.

In one assay format, the expression of a nucleic acid encoding a gene of the invention (see Tables 4, 5 and 6) in a cell or tissue sample is monitored directly by hybridization to the nucleic acids of the invention. Cell lines or tissues are exposed to the agent to be tested under appropriate conditions and time and total RNA or mRNA is isolated by standard procedures such as those disclosed in Sambrook et al., (1989) Molecular Cloning: A Laboratory Manual, Cold Spring Harbor Laboratory Press).

Probes to detect differences in RNA expression levels between cells exposed to the agent and control cells may be prepared as described above. Hybridization conditions are modified using known methods, such as those described by Sambrook et al., and Ausubel et al., as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The chip or wafer can then be exposed to total cellular RNA or polyA RNA from a sample under conditions in which the affixed sequences will specifically hybridize to the RNA. By examining for the ability of a given probe to specifically hybridize to an RNA sample from an untreated cell population and from a cell population exposed to the agent, agents which up or down regulate expression are identified.

Methods to Identify Agents that Modulate the Activity of a Protein Encoded by a Gene Involved in Longevity.

The present invention provides methods for identifying agents that modulate at least one activity of the proteins described in Tables 4, 5 and 6. Such methods may utilize any means of monitoring or detecting the desired activity. As used herein, an agent is said to modulate the expression of a protein of the invention if it is capable of up- or down-regulating expression of the protein in a cell. Such cells can be obtained from any parts of the body such as the scalp, blood, dermis, epidermis and other skin cells, cutaneous surfaces, intertrigious areas, genitalia, vessels and endothelium. Some non-limiting examples of cells that can be used are red blood cells, muscle cells, heart cells, nerve cells, insulin-producing cells, pancreatic cells, brain cells, germ cells, keratinocytes, monocytes, neutrophils, langerhans cells, CD4+ and CD8+ T cells, B and T lymphocytes, leukocytes, hormonal cells, bone marrow cells, skin cells, buccal cells, spinal cord cells, bone cells, adipose cells, cartilage cells, dendritic cells, intestinal cells, hepatic cells, mucous cells, olfactory cells, retinal cells, somatic cells and arterial cells.

In one format, the specific activity of a protein of the invention, normalized to a standard unit, may be assayed in a cell population that has been exposed to the agent to be tested and compared to an unexposed control cell population may be assayed. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes can be prepared by immunizing suitable mammalian hosts utilizing appropriate immunization protocols using the proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. (Rockford, Ill.) may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation. While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) or modifications which affect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid. The desired monoclonal antibodies may be recovered from the culture supernatant or from the ascites supernatant. Fragments of the monoclonal antibodies or the polyclonal antisera which contain the immunologically significant portion(s) can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin. The antibodies or fragments may also be produced, using current technology, by recombinant means. Antibody regions that bind specifically to the desired regions of the protein can also be produced in the context of chimeras derived from multiple species. Antibody regions that bind specifically to the desired regions of the protein can also be produced in the context of chimeras from multiple species, for instance, humanized antibodies. The antibody can therefore be a humanized antibody or a human antibody, as described in U.S. Pat. No. 5,585,089 or Riechmann et al. (1988).

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use of a chemical library or a peptide combinatorial library, or a growth broth of an organism. As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. The agents of the present invention can be, as examples, oligonucleotides, antisense polynucleotides, interfering RNA, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention includes antibodies or fragments thereof that bind to a protein encoded by a gene in Tables 4, 5 and 6. Antibody agents can be obtained by immunization of suitable mammalian subjects with peptides, containing as antigenic regions, those portions of the protein intended to be targeted by the antibodies (see section above of antibodies as probes for standard antibody preparation methodologies).

In yet another class of agents, the present invention includes peptide mimetics that mimic the three-dimensional structure of the protein encoded by a gene from Tables 4, 5 and 6. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others. In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In another form, peptide analogs are commonly used in the pharmaceutical industry as non-peptide drugs with properties analogous to those of the template peptide. These types of non-peptide compounds are also referred to as peptide mimetics or peptidomimetics (Fauchere, 1986; Veber & Freidinger, 1985; Evans et al., 1987) which are usually developed with the aid of computerized molecular modeling. Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage using methods known in the art. Labeling of peptide mimetics usually involves covalent attachment of one or more labels, directly or through a spacer (e.g., an amide group), to non-interfering position(s) on the peptide mimetic that are predicted by quantitative structure-activity data and molecular modeling. Such non-interfering positions generally are positions that do not form direct contacts with the macromolecule(s) to which the peptide mimetic binds to produce the therapeutic effect. Derivitization (e.g., labeling) of peptide mimetics should not substantially interfere with the desired biological or pharmacological activity of the peptide mimetic. The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al., 1991) and the phage display method. The two hybrid method detects protein-protein interactions in yeast (Fields et al., 1989). The phage display method detects the interaction between an immobilized protein and a protein that is expressed on the surface of phages such as lambda and M13 (Amberg et al., 1993; Hogrefe et al., 1993). These methods allow positive and negative selection for protein-protein interactions and the identification of the sequences that determine these interactions.

### Method to Diagnose Longevity Trait and Age-Related Disease

The present invention also relates to methods for diagnosing longevity trait or a related disorder, preferably age-related diseases, a disposition to such trait, predisposition to such a trait and/or disorder progression. In some methods, the steps comprise contacting a target sample with (a) nucleic molecule(s) or fragments thereof and comparing the concentration of individual mRNA(s) with the concentration of the corresponding mRNA(s) from at least one healthy donor. An aberrant (increased or decreased) mRNA level of at least one gene from Tables 4, 5 and 6, at least 5 or 10 genes from Tables 4, 5 and 6, at least 20 genes from Tables 4, 5 and 6, at least 30 genes from Tables 4, 5 and 6 determined in the sample in comparison to the control sample is an indication of longevity or a related disorder or a disposition to such kinds of disorders. For diagnosis, samples are from any parts of the body such as the scalp, blood, dermis, epidermis and other skin cells, cutaneous surfaces, intertrigious areas, genitalia, vessels and endothelium. Some non-limiting examples of cells that can be used are red blood cells, muscle cells, heart cells, nerve cells, insulin-producing cells, pancreatic cells, brain cells, germ cells, keratinocytes, monocytes, neutrophils, langerhans cells, CD4+ and CD8+ T cells, B and T lymphocytes, leukocytes, hormonal cells, bone marrow cells, skin cells, buccal cells, spinal cord cells, bone cells, adipose cells, cartilage cells, dendritic cells, intestinal cells, hepatic cells, mucous cells, olfactory cells, retinal cells, somatic cells and arterial cells.

For analysis of gene expression, total RNA is obtained from cells according to standard procedures and, preferably, reverse-transcribed. Preferably, a DNAse treatment (in order to get rid of contaminating genomic DNA) is performed. Some non-limiting examples of cells that can be used are: red blood cells, muscle cells, heart cells, nerve cells, insulin-producing cells, pancreatic cells, brain cells, germ cells, keratinocytes, monocytes, neutrophils, langerhans cells, CD4+ and CD8+ T cells, B and T lymphocytes, leukocytes, hormonal cells, bone marrow cells, skin cells, buccal cells, spinal cord cells, bone cells, adipose cells, cartilage cells, dendritic cells, intestinal cells, hepatic cells, mucous cells, olfactory cells, retinal cells, somatic cells and arterial cells.

The nucleic acid molecule or fragment is typically a nucleic acid probe for hybridization or a primer for PCR. The person skilled in the art is in a position to design suitable nucleic acids probes based on the information provided in the Tables of the present invention. The target cellular component, i.e. mRNA, e.g., in skin, may be detected directly in situ, e.g. by in situ hybridization or it may be isolated from other cell components by common methods known to those skilled in the art before contacting with a probe. Detection methods include Northern blot analysis, RNase protection, in situ methods, e.g. in situ hybridization, in vitro amplification methods (PCR, LCR, QRNA replicase or RNA-transcription/amplification (TAS, 3SR), reverse dot blot disclosed in EP-B10237362) and other detection assays that are known to those skilled in the art. Products obtained by in vitro amplification can be detected according to established methods, e.g. by separating the products on agarose or polyacrylamide gels and by subsequent staining with ethidium bromide. Alternatively, the amplified products can be detected by using labeled primers for amplification or labeled dNTPs. Preferably, detection is based on a microarray.

The probes (or primers) (or, alternatively, the reverse-transcribed sample mRNAs) can be detectably labeled, for example, with a radioisotope, a bioluminescent compound, a chemiluminescent compound, a fluorescent compound, a metal chelate, or an enzyme.

The present invention also relates to the use of the nucleic acid molecules or fragments described above for the preparation of a diagnostic composition for the diagnosis of longevity or a disposition to such a trait.

The present invention also relates to the use of the nucleic acid molecules of the present invention for the isolation or development of a compound which is useful for therapy of age-associated diseases. For example, the nucleic acid molecules of the invention and the data obtained using said nucleic acid molecules for diagnosis of longevity trait might allow for the identification of further genes which are specifically dysregulated, and thus may be considered as potential targets for therapeutic interventions.

The invention further provides prognostic assays that can be used to identify subjects having or at risk of developing age-associated diseases. In such method, a test sample is obtained from a subject and the amount and/or concentration of the nucleic acid described in Tables 4, 5 and 6 is determined; wherein the presence of an associated allele, a particular allele of a polymorphic locus, or the likes in the nucleic acids sequences of this invention (see SEQ ID from Tables 2-7) can be diagnostic for a subject having or at risk of developing age-associated diseases. As used herein, a “test sample” refers to a biological sample obtained from a subject of interest. For example, a test sample can be a biological fluid, a cell sample, or tissue. A biological fluid can be, but is not limited to saliva, serum, mucus, urine, stools, spermatozoids, vaginal secretions, lymph, amiotic liquid, pleural liquid and tears. Some non-limiting examples of cells that can be used are red blood cells, muscle cells, heart cells, nerve cells, insulin-producing cells, pancreatic cells, brain cells, germ cells, keratinocytes, monocytes, neutrophils, langerhans cells, CD4+ and CD8+ T cells, B and T lymphocytes, leukocytes, hormonal cells, bone marrow cells, skin cells, buccal cells, spinal cord cells, bone cells, adipose cells, cartilage cells, dendritic cells, intestinal cells, hepatic cells, mucous cells, olfactory cells, retinal cells, somatic cells and arterial cells.

Furthermore, the prognostic assays described herein can be used to determine whether a subject can be administered an agent (e.g., an agonist, antagonist, peptidomimetic, polypeptide, nucleic acid such as antisense DNA or interfering RNA (RNAi), small molecule or other drug candidate) to treat age-associated diseases. Specifically, these assays can be used to predict whether an individual will have an efficacious response or will experience adverse events in response to such an agent. For example, such methods can be used to determine whether a subject can be effectively treated with an agent that modulates the expression and/or activity of a gene from Tables 4, 5 and 6, or the nucleic acids described herein. In another example, an association study may be performed to identify polymorphisms from Tables 2, 3 and 7 that are associated with a given response to the agent e.g., an efficacious response or the likelihood of one or more adverse events. Thus, the present invention provides methods for determining whether a subject can be effectively treated with an agent for a disorder associated with aberrant expression or activity of a gene from Tables 4, 5 and 6 in which a test sample is obtained and nucleic acids or polypeptides from Tables 2, 3 or 7 are detected (e.g., wherein the presence of a particular level of expression of a gene from Tables 4, 5 and 6 or a particular allelic variant of such gene, such as polymorphism from Tables 2, 3 or 7, is diagnostic for a subject that can be administered an agent to treat a trait or age-associated disease). In one embodiment, the method includes obtaining a sample from a subject suspected of having age-associated diseases or an affected individual and exposing such sample to an agent. The expression and/or activity of the nucleic acids and or genes of the invention are monitored before and after treatment with such agent to assess the effect of such agent. After analysis of the expression values, one skilled in the art can determine whether such agent can effectively treat such subject. In another embodiment, the method includes obtaining a sample from a subject having or susceptible to developing an age-associated disease and determining the allelic constitution of one or more polymorphism from Tables 2, 3 or 7 that are associated with a particular response to an agent. After analysis of the allelic constitution of the individual at the associated polymorphisms, one skilled in the art can determine whether such agent can effectively treat such subject.

The methods of the invention can also be used to detect genetic alterations in a gene from Tables 4, 5 and 6, thereby determining if a subject with the lesioned gene is at risk for an age-associated disorder. In preferred embodiments, the methods include detecting, in a sample of cells from the subject, the presence or absence of a genetic alteration characterized by at least one alteration linked to or affecting the integrity of a gene from Tables 4, 5 and 6 encoding a polypeptide or the misexpression of such gene. For example, such genetic alterations can be detected by ascertaining the existence of at least one of: (1) a deletion of one or more nucleotides from a gene from Tables 4, 5 and 6; (2) an addition of one or more nucleotides to a gene from Tables 4, 5 and 6; (3) a substitution of one or more nucleotides of a gene from Tables 4, 5 and 6; (4) a chromosomal rearrangement of a gene from Tables 4, 5 and 6; (5) an alteration in the level of a messenger RNA transcript of a gene from Tables 4, 5 and 6; (6) aberrant modification of a gene from Tables 4, 5 and 6, such as of the methylation pattern of the genomic DNA, (7) the presence of a non-wild type splicing pattern of a messenger RNA transcript of a gene from Tables 4, 5 and 6; (8) inappropriate post-translational modification of a polypeptide encoded by a gene from Tables 4, 5 and 6; and (9) alternative promoter use. As described herein, there are a large number of assay techniques known in the art which can be used for detecting alterations in a gene from Tables 4, 5 and 6. A preferred biological sample is a peripheral blood sample obtained by conventional means from a subject. Another preferred biological sample is a buccal swab. Other biological samples can be, but is not limited to, urine, stools, spermatozoids, vaginal secretions, lymph, amniotic liquid, pleural liquid and tears. In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or alternatively, in a ligation chain reaction (LCR) (see, e.g., Landegran et al., 1988; and Nakazawa et al., 1994), the latter of which can be particularly useful for detecting point mutations in a gene from Tables 4, 5 and 6 (see Abavaya et al., 1995). This method can include the steps of collecting a sample of cells from a patient, isolating nucleic acid (e.g., genomic DNA, mRNA, or both) from the cells of the sample, contacting the nucleic acid sample with one or more primers which specifically hybridize to a gene from Tables 4, 5 and 6 under conditions such that hybridization and amplification of the nucleic acid from Tables 4, 5 and 6 (if present) occurs, and detecting the presence or absence of an amplification product, or detecting the size of the amplification product and comparing the length to a control sample. PCR and/or LCR may be desirable to use as a preliminary amplification step in conjunction with some of the techniques used for detecting an associated allele, a particular allele of a polymorphic locus, or the like described herein.

Alternative amplification methods include: self sustained sequence replication (Guatelli et al., 1990), transcriptional amplification system (Kwoh et al., 1989), Q-Beta Replicase (Lizardi et al., 1988), isothermal amplification (e.g. Dean et al., 2002); and Hafner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

In an alternative embodiment, alterations in a gene from Tables 4, 5 and 6, from a sample cell can be identified by identifying changes in a restriction enzyme cleavage pattern. For example, sample and control DNA is isolated, amplified (optionally), digested with one or more restriction endonucleases, and fragment length sizes are determined by gel electrophoresis and compared. Differences in fragment length sizes between sample and control DNA indicates an associated allele, a particular allele of a polymorphic locus, or the like, in the sample DNA. Moreover, sequence specific ribozymes (see, e.g., U.S. Pat. No. 5,498,531 or DNAzyme e.g. U.S. Pat. No. 5,807,718) can be used to score for the presence of specific associated allele, a particular allele of a polymorphic locus, or the likes by development or loss of a ribozyme or DNAzyme cleavage site.

The present invention also relates to further methods for diagnosing the longevity trait or a related disorder, preferably an age-associated disorder, a disposition to such disorder, and predisposition to such a disorder and/or disorder progression. In some methods, the steps comprise contacting a target sample with (a) nucleic molecule(s) or fragments thereof and determining the presence or absence of a particular allele of a polymorphism that confers a disorder-related phenotype (e.g., predisposition to such a disorder and/or disorder progression). The presence of at least one allele from Tables 2, 3 or 7 that is associated with the longevity trait (“associated allele”), at least 5 or 10 associated alleles from Tables 2, 3 or 7, at least 50 associated alleles from Tables 2, 3 or 7, at least 100 associated alleles from Tables 2, 3 or 7, or at least 200 associated alleles from Tables 2, 3 or 7 determined in the sample is an indication of the longevity trait or a related age-associated disorder, a disposition or predisposition to such kinds of disorders, or a prognosis for such disorder progression. Samples may be obtained from any parts of the body such as the scalp, blood, dermis, epidermis and other skin cells, cutaneous surfaces, intertrigious areas, genitalia, vessels and endothelium. Some non-limiting examples of cells that can be used are red blood cells, muscle cells, heart cells, nerve cells, insulin-producing cells, pancreatic cells, brain cells, germ cells, keratinocytes, monocytes, neutrophils, langerhans cells, CD4+ and CD8+ T cells, lymphocytes, hormonal cells, bone marrow cells, skin cells, buccal cells, spinal cord cells, bone cells, adipose cells, cartilage cells, dendritic cells, intestinal cells, hepatic cells, mucous cells, olfactory cells, retinal cells, somatic cells and arterial cells.

In other embodiments, alterations in a gene from Tables 4, 5 and 6 or a locus from Tables 2, 3 or 7, or different alleles of the polymorphisms from Tables 2, 3 or 7 can be identified by hybridizing sample and control nucleic acids, e.g., DNA or RNA, to high density arrays or bead arrays containing tens to thousands of oligonucleotide probes (Cronin et al., 1996; Kozal et al., 1996). For example, alterations in a gene from Tables 4, 5 and 6 or a locus from Tables 2, 3 or 7, or different alleles of the polymorphisms from Tables 2, 3 or 7 can be identified in two dimensional arrays containing light-generated DNA probes as described in Cronin et al., (1996). Briefly, a first hybridization array of probes can be used to scan through long stretches of DNA in a sample and control to identify base changes between the sequences by making linear arrays of sequential overlapping probes. This step allows the identification of point mutations or different alleles of polymorphisms. This step is followed by a second hybridization array that allows the characterization of specific mutations, associated alleles or alleles of a particular polymorphic locus, by using smaller, specialized probe arrays complementary to all variants or mutations detected. Each mutation array is composed of parallel probe sets, one complementary to the wild-type gene and the other complementary to the mutant gene or associated alleles or particular allele of a polymorphic locus.

In yet another embodiment, any of a variety of sequencing reactions known in the art can be used to directly sequence a gene from Tables 4, 5 and 6 and detect an associated allele, a particular allele of a polymorphic locus, or the like by comparing the sequence of the sample gene from Tables 4, 5 and 6 with the corresponding wild-type (control) sequence. Examples of sequencing reactions include those based on techniques developed by Maxam and Gilbert (1977) or Sanger (1977). It is also contemplated that any of a variety of automated sequencing procedures can be utilized when performing the diagnostic assays (Bio/Techniques 19:448, 1995) including sequencing by mass spectrometry (see, e.g. PCT International Publication No. WO 94/16101; Cohen et al., 1996; and Griffin et al. 1993), real-time pyrophosphate sequencing method (Ronaghi et al., 1998; and Permutt et al., 2001) and sequencing by hybridization (see e.g. Drmanac et al., 2002).

Other methods of detecting an associated allele, a particular allele of a polymorphic locus, or the likes in a gene from Tables 4, 5 and 6 include methods in which protection from cleavage agents is used to detect mismatched bases in RNA/RNA, DNA/DNA or RNA/DNA heteroduplexes (Myers et al., 1985). In general, the art technique of “mismatch cleavage” starts by providing heteroduplexes formed by hybridizing (labeled) RNA or DNA containing the wild-type gene from Tables 4, 5 and 6 sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of an associated allele, a particular allele of a polymorphic locus, or the like. (see, for example, Cotton et al., 1988; Saleeba et al., 1992). In a preferred embodiment, the control DNA or RNA can be labeled for detection, as described herein.

In still another embodiment, the mismatch cleavage reaction employs one or more proteins that recognize mismatched base pairs in double-stranded DNA (so called “DNA mismatch repair” enzymes) in defined systems for detecting and mapping point an associated allele, a particular allele of a polymorphic locus, or the likes in a gene from Tables 4, 5 and 6 cDNAs obtained from samples of cells. For example, the mutY enzyme of E. coli cleaves A at G/A mismatches (Hsu et al., 1994). Other examples include, but are not limited to, the MutHLS enzyme complex of E. coli (Smith and Modrich., 1996) and Cel 1 from the celery (Kulinski et al., 2000) both cleave the DNA at various mismatches. According to an exemplary embodiment, a probe based on a gene sequence from Tables 4, 5 and 6 is hybridized to a cDNA or other DNA product from a test cell or cells. The duplex is treated with a DNA mismatch repair enzyme, and the cleavage products, if any, can be detected using electrophoresis protocols or the like. See, for example, U.S. Pat. No. 5,459,039. Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad et al., 2004).

In other embodiments, alterations in electrophoretic mobility can be used to identify an associated allele, a particular allele of a polymorphic locus, or the likes in genes from Tables 4, 5 and 6. For example, single strand conformation polymorphism (SSCP) can be used to detect differences in electrophoretic mobility between mutant and wild type nucleic acids (Orita et al., 1993; see also Cotton, 1993; and Hayashi et al., 1992). Single-stranded DNA fragments of sample and control nucleic acids from Tables 4, 5 and 6 will be denatured and allowed to renature. The secondary structure of single-stranded nucleic acids varies according to sequence; the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et al., 1985). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoorn et al., 2000).

Examples of other techniques for detecting point mutations, associated alleles or alleles of a particular polymorphic locus include, but are not limited to, selective oligonucleotide hybridization, selective amplification, selective primer extension, selective ligation, single-base extension, selective termination of extension or invasive cleavage assay. For example, oligonucleotide primers may be prepared in which the known associated allele, particular allele of a polymorphic locus, or the like is placed centrally and then hybridized to target DNA under conditions which permit hybridization only if a perfect match is found (Saiki et al., 1986; Saiki et al., 1989). Such allele specific oligonucleotides are hybridized to PCR amplified target DNA or a number of different associated allele, a particular allele of a polymorphic locus, or the likes where the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like locus is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

The hybridization may also be carried out with a temperature gradient following the principle of dynamic allele-specific hybridization or like (e.g. Jobs et al., 2003; and Bourgeois and Labuda, 2004). For example, the hybridization is done using one of the two allele-specific oligonucleotides labeled with a fluorescent dye, an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of the associated allele, particular allele of a polymorphic locus, or the like (e.g. Jobs et al. supra). Alternatively, the hybridization is done under a gradually decreasing temperature. In this case, both allele-specific oligonucleotides are hybridized to the template competitively. At high temperature none of the two probes is hybridized. Once the optimal temperature of the full-matched probe is reached, it hybridizes and leaves no target for the mismatched probe. In the latter case, if the allele-specific probes are differently labeled, then they are hybridized to a single PCR-amplified target. If the probes are labeled with the same dye, then the probe cocktail is hybridizes twice to identical templates with only one labeled probes, different in the two cocktails, in the presence of the unlabeled competitive probe.

Alternatively, allele specific amplification technology that depends on selective PCR amplification may be used in conjunction with the present invention. Oligonucleotides used as primers for specific amplification may carry the associated allele, particular allele of a polymorphic locus, or the like of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the associated allele, particular allele of a polymorphic locus, or the like to create cleavage-based detection (Gasparini et al., 1992). It is anticipated that in certain embodiments amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., 1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNP and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78, 2003).

Another alternative includes the single-base extension/ligation assay using a molecular inversion probe, consisting of a single, long oligonucleotide (see e.g. Hardenbol et al., 2003). In such an embodiment, the oligonucleotide hybridizes on both side of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, bead array or like. This reaction can be performed at a highly multiplexed level.

In another alternative, the associated allele, particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, bead array or like. In the latter case, the extension oligonucleotide will contain a SNP-specific zip code tag.

In yet another alternative, the variant is scored by selective termination of extension. The template is first amplified by PCR and the extension oligonucleotide hybridizes in vicinity to the SNP locus, close to but not necessarily adjacent to it. The extension reaction is carried out using a thermostable polymerase such as Thermo Sequenase (GE Healthcare) in the presence of a mix of dNTPs and at least one ddNTP. The latter has to terminate the extension at one of the alleles of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, the associated allele, particular allele of a polymorphic locus, or the like is detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Other types of markers can also be used for diagnostic purposes. For example, microsatellites can also be useful to detect the genetic predisposition of an individual to a given disorder. Microsatellites consist of short sequence motifs of one or a few nucleotides repeated in tandem. The most common motifs are polynucleotide runs, dinucleotide repeats (particularly the CA repeats) and trinucleotide repeats. However, other types of repeats can also be used. The microsatellites are very useful for genetic mapping because they are highly polymorphic in their length. Microsatellite markers can be typed by various means, including but not limited to DNA PCR fragment sizing, oligonucleotide ligation assay and mass spectrometry. For example, the locus of the microsatellite is amplified by PCR and the size of the PCR fragment will be directly correlated to the length of the microsatellite repeat. The size of the PCR fragment can be detected by regular means of gel electrophoresis. The fragment can be labeled internally during PCR or by using end-labeled oligonucleotides in the PCR reaction (e.g. Mansfield et al., 1996). Alternatively, the size of the PCR fragment is determined by mass spectrometry. In such a case, however, the flanking sequences need to be eliminated. This can be achieved by ribozyme cleavage of an RNA transcript of the microsatellite repeat (Krebs et al., 2001). For example, the microsatellite locus is amplified using oligonucleotides that include a T7 promoter on one end and a ribozyme motif on the other end. Transcription of the amplified fragments will yield an RNA substrate for the ribozyme, releasing small RNA fragments that contain the repeated region. The size of the latter is determined by mass spectrometry. Alternatively, the flanking sequences are specifically degraded. This is achieved by replacing the dTTP in the PCR reaction by dUTP. The dUTP nucleosides are then removed by uracyl DNA glycosylases and the resulting abasic sites are cleaved by either abasic endonucleases such as human AP endonuclease or chemical agents such as piperidine. Bases can also be modified post-PCR by chemical agents such as dimethyl sulfate and then cleaved by other chemical agents such as piperidine (see e.g. Maxam and Gilbert, 1977; U.S. Pat. No. 5,869,242; and U.S. Patent pending Ser. No. 60/335,068).

In another alternative, an oligonucleotide ligation assay can be performed. The microsatellite locus is first amplified by PCR. Then, different oligonucleotides can be submitted to ligation at the center of the repeat with a set of oligonucleotides covering all the possible lengths of the marker at a given locus (Zirvi et al., 1999). Another example of design of an oligonucleotide assay comprises the ligation of three oligonucleotides; a 5′ oligonucleotide hybridizing to the 5′ flanking sequence, a repeat oligonucleotide of the length of the shortest allele of the marker hybridizing to the repeated region and a set of 3′ oligonucleotides covering all the existing alleles hybridizing to the 3′ flanking sequence and a portion of the repeated region for all the alleles longer than the shortest one. For the shortest allele, the 3′ oligonucleotide exclusively hybridizes to the 3′ flanking sequence (U.S. Pat. No. 6,479,244).

The methods described herein may be performed, for example, by utilizing pre-packaged diagnostic kits comprising at least one probe nucleic acid selected from the SEQ ID of Tables 2-7, or antibody reagent described herein, which may be conveniently used, for example, in a clinical setting to diagnose patient exhibiting symptoms or a family history of a disorder or genetic trait, or disorder involving abnormal activity of genes from Tables 4, 5 and 6.

### Method to Treat an Animal Suspected of Having Age-Associated Diseases

The present invention provides methods of treating a disorder associated with the longevity trait, such age-associated diseases by expressing in vivo the nucleic acids of at least one gene from Tables 4, 5 and 6. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids encoding a gene from Tables 4, 5 and 6, under the control of a promoter, then expresses the encoded protein, thereby mitigating the effects of absent, partial inactivation, or abnormal expression of a gene from Tables 4, 5 and 6.

Such gene therapy procedures have been used to correct acquired and inherited genetic defects, cancer, and viral infection in a number of contexts. The ability to express artificial genes in humans facilitates the prevention and/or cure of many important human disorders, including many disorders which are not amenable to treatment by other therapies (for a review of gene therapy procedures, see Anderson, 1992; Nabel & Felgner, 1993; Mitani & Caskey, 1993; Mulligan, 1993; Dillon, 1993; Miller, 1992; Van Brunt, 1998; Vigne, 1995; Kremer & Perricaudet 1995; Doerfler & Bohm 1995; and Yu et al., 1994).

Delivery of the gene or genetic material into the cell is the first critical step in gene therapy treatment of disorder or genetic traits. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see the references included in the above section.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vector that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., 1992; Johann et al., 1992; Sommerfelt et al., 1990; Wilson et al., 1989; Miller et al., 1999; and PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., 1987; U.S. Pat. No. 4,797,368; WO 93/24641; Kotin, 1994; Muzyczka, 1994). Construction of recombinant AAV vectors are described in a number of publications, including U.S. Pat. No. 5,173,414; Tratschin et al., 1985; Tratschin, et al., 1984; Hermonat & Muzyczka, 1984; and Samulski et al., 1989.

In particular, numerous viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors (Ellem et al., 1997; and Dranoff et al., 1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., 1998, Kearns et al 1996).

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., 1998). Additional examples of the use of adenovirus vectors for gene transfer in clinical trials include Rosenecker et al., 1996; Sterman et al., 1998; Welsh et al., 1995; Alvarez et al., 1997; Topf et al., 1998.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, and tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., 1994; and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+(panB cells), GR-1 (granulocytes), and lad (differentiated antigen presenting cells).

Vectors (e.g., retroviruses, adenoviruses, liposomes, etc.) containing therapeutic nucleic acids can be also administered directly to the organism for transduction of cells in vivo. Alternatively, naked DNA can be administered.

Administration is by any of the routes normally used for introducing a molecule into ultimate contact with blood or tissue cells, as described above. The nucleic acids from Tables 2-7 are administered in any suitable manner, preferably with the pharmaceutically acceptable carriers described above. Suitable methods of administering such nucleic acids are available and well known to those of skill in the art, and, although more than one route can be used to administer a particular composition, a particular route can often provide a more immediate and more effective reaction than another route (see Samulski et al., 1989). The present invention is not limited to any method of administering such nucleic acids, but preferentially uses the methods described herein.

The present invention further provides other methods of treating disorders, such as age-associated disorders, by for example administering to an individual having an age-associated disorder (or suspected of having a age-associated disorder) an effective amount of an agent that regulates the expression, activity or physical state of at least one gene from Tables 4, 5 and 6. An “effective amount” of an agent is an amount that modulates a level of expression or activity of a gene from Tables 4, 5 and 6, in a cell in the individual at least about 10%, at least about 20%, at least about 30%, at least about 40%, at least about 50%, at least about 60%, at least about 70%, at least about 80% or more, compared to a level of the respective gene from Tables 4, 5 and 6 in a cell in the individual in the absence of the compound. The preventive or therapeutic agents of the present invention may be administered, either orally or parenterally, systemically or locally. For example, intravenous injection such as drip infusion, intramuscular injection, intraperitoneal injection, subcutaneous injection, suppositories, intestinal lavage, oral enteric coated tablets, and the like can be selected, and the method of administration may be chosen, as appropriate, depending on the age and the conditions of the patient. The effective dosage is chosen from the range of 0.01 mg to 100 mg per kg of body weight per administration. Alternatively, the dosage in the range of 1 to 1000 mg, preferably 5 to 50 mg per patient may be chosen. The therapeutic efficacy of the treatment may be monitored by observing various parts of the body and mind, such as the response to a cognitive test, by any monitoring method known in the art, such as the MMSE (mini-mental state examination). Others ways of monitoring efficacy can be, but are not limited to monitoring well-being, memory, mental state and psychological state of the patient.

The present invention further provides a method of treating an individual clinically diagnosed with a trait or age-associated disorder. The methods generally comprises analyzing a biological sample that includes a cell, in some cases, a skin cell, from an individual clinically diagnosed with an age-associated disorder for the presence of modified levels of expression of at least 1 gene, at least 10 genes, at least 30 genes from Tables 4, 5 and 6. A treatment plan that is most effective for individuals clinically diagnosed as having a condition associated with a trait or age-associated disorder is then selected on the basis of the detected expression of such genes in a cell. Treatment may include administering a composition that includes an agent that modulates the expression or activity of a protein from Tables 4, 5 and 6 in the cell. Information obtained as described in the methods above can also be used to predict the response of the individual to a particular agent. Thus, the invention further provides a method for predicting a patient's likelihood to respond to a drug treatment for a condition associated with the longevity trait, comprising determining whether modified levels of a gene from Tables 4, 5 and 6 is present in a cell, wherein the presence of protein is predictive of the patient's likelihood to respond to a drug treatment for the condition. Examples of the prevention or improvement of symptoms accompanied by age-associated disorders that can monitor for effectiveness include response to a cognitive test, by any monitoring method known in the art, such as the MMSE (mini-mental state examination). Others ways of monitoring efficacy can be, but are not limited to monitoring well-being, memory, mental state and psychological state of the patient.

The invention also provides a method of predicting a response to therapy in a subject having age-associated disorders by determining the presence or absence in the subject of one or more markers associated with the longevity trait described in Tables 2, 3 and/or 7, diagnosing the subject in which the one or more markers are present as having an age-associated disorder, and predicting a response to a therapy based on the diagnosis e.g., response to therapy may include an efficacious response and/or one or more adverse events. The invention also provides a method of optimizing therapy in a subject having an age-associated disorder by determining the presence or absence in the subject of one or more markers associated with a clinical subtype of age-associated disorders, diagnosing the subject in which the one or more markers are present as having a particular clinical subtype of age-associated disorders, and treating the subject having a particular clinical subtype of age-associated disorders based on the diagnosis.

Thus, while there are a number of treatments for age-associated disorders currently available, they all are accompanied by various side effects, high costs, and long complicated treatment protocols, which are often not available and effective in a large number of individuals. Accordingly, there remains a need in the art for more effective and otherwise improved methods for treating and preventing age-associated disorders. Thus, there is a continuing need in the medical arts for genetic markers of longevity trait and guidance for the use of such markers. The present invention fulfills this need and provides further related advantages.

### EXAMPLES

### Example 1

### Identification of Cases and Controls

All individuals were sampled from the Quebec founder population (QFP). Membership in the founder population was defined as having four grandparents with French Canadian family names who were born in the Province of Quebec, Canada or in adjacent areas of the Provinces of New Brunswick and Ontario or in New England or New York State. The Quebec founder population has two distinct advantages over general populations for LD mapping. Because it is relatively young, about 12 to 15 generations from mid-17th century to present, and because it has a limited but sufficient number of founders, approximately 2600 effective founders (Charbonneau et al. 1987), the Quebec population is characterized both by extended LD and by decreased genetic heterogeneity. The increased extent of LD allows the detection of genes affecting the trait using a reasonable marker density, while still allowing the increased meiotic resolution of population-based mapping. The number of founders is small enough to result in increased LD and reduced allelic heterogeneity, yet large enough to insure that all of the major genes affecting the trait involved in general populations are present in Quebec. Reduced allelic heterogeneity will act to increase relative risk imparted by the remaining alleles and so increase the power of case/control studies to detect genes and trait associated alleles within the Quebec population. The specific combination of age in generations, optimal number of founders and large present population size makes the QFP optimal for LD-based gene mapping. The family relationships among samples are routinely examined using proprietary algorithms and information from the genealogical data bases. When two subjects are found to be too closely related for LD analysis, one of them is removed from the sample.

Case inclusion criteria for the study included being 94 years of age or older. Control inclusion criterion for the study included being 65 years of age or younger and gender matched to cases.

All human sampling was subject to ethical review procedures.

All enrolled QFP subjects (cases and controls) provided a 30 ml blood sample (3 barcoded tubes of 10 ml). Samples were processed immediately upon arrival at Genizon's laboratory. All samples were scanned and logged into a LabVantage Laboratory Information Management System (LIMS), which served as a hub between the clinical data management system and the genetic analysis system. Following centrifugation, the buffy coat containing the white blood cells was isolated from each tube. Genomic DNA was extracted from the buffy coat from one of the tubes, and stored at 4° C. until required for genotyping. DNA extraction was performed with a commercial kit using a guanidine hydrochloride based method (FlexiGene, Qiagen) according to the manufacturer's instructions. The extraction method yielded high molecular weight DNA, and the quality of every DNA sample was verified by agarose gel electrophoresis. Genomic DNA appeared on the gel as a large band of very high molecular weight. The remaining two buffy coats were stored at −80° C. as backups.

The samples were collected as 615 cases and 615 controls (127 males and 488 females, for both cases and controls). The DNA extracted from cases and control samples was pooled together in various case and control pools.

Separate case and control and male and female pools were constructed. The probands are also segregated according to their age at the time of recruitment and the proband females are further separated in two groups, those who failed a cognitive test and those who passed the test. Two proband male pools contain 53-74 individuals, separated by age group. One proband female pool contains the 71 females who failed a cognitive test whereas the 7 remaining pools consisted of 43-80 proband females who passed the test, separated by age group. Ten (10) control pools consisted of 8 pools of 61 female samples and 2 pools of 63-64 male samples.

### Example 2

### Genome Wide Association

Genotyping was performed using Perlegen Life Sciences ultra-high-throughput platform. Loci of interest were amplified and hybridized to wafers containing arrays of oligonucleotides. Allele discrimination was performed through allele-specific hybridization. In total, 248,535 SNPs, spread over 3 microarrays, were genotyped. This set of markers contained the QLDM (Quebec LD Map), a map created specifically for the Quebec founder population, which possesses a base density of one marker per 40 kb and up to one marker per 10 kb in low-LD regions, the lower the LD is in a given area, the higher the marker density will be. The QLDM markers and other markers were selected from various databases including the ˜1.6 million SNP database of Perlegen Life Sciences (Patil, 2001), the hapmap consortium database and dbSNP at NCBI. The SNPs were chosen to maximize uniformity of genetic coverage and as much as possible, with a minor allele frequency of 10% or higher.

The genotyping information was entered into a Unified Genotype Database (a proprietary database under development) from which it was accessed using custom-built programs for export to the genetic analysis pipeline. Analyses of these genotypes were performed with the statistical tools described in Example 3. The GWS permitted the identification of 47 candidate regions that are further analyzed by the Confirmation Mapping and Fine Mapping approaches described below.

### Example 3

### Genetic Analysis

The raw data generated by the GWS approach (Example 2 herein) was analyzed by various means to identify candidate regions (see also Confirmatory Mapping and Fine Mapping described in Example 5).

### Raw Data Analysis by Perlegen

The data analysis process compares the relative fluorescence intensities of features corresponding to the reference allele of a given SNP with those corresponding to the alternate allele, to calculate a p-hat value. The latter is proportional to the fluorescence signal from perfect match features for the reference allele divided by the sum of fluorescence signals from perfect match features for the reference plus the alternate alleles. P-hat assumes values close to 1 (typically 0.9) for pure reference samples and close to 0 (typically 0.1) for pure alternate samples, and can be used as a measured estimate of the reference allele frequency of a SNP in a DNA pool. The difference between case and control pools, delta p-hat, is calculated using the weighted average of case and control p-hats. Delta p-hat is a reliable estimate of the allele frequency difference between the cases and controls.

### Data Analysis by Genizon

Analysis of the data by Genizon was based upon Perlegen's p-hat values.

### Data Analysis of P-Hat

The data consisted of 10 pools of cases and 10 pools of controls, with a p-hat value for each pool (provided by Perlegen's analysis methods described above). The weighted mean p-hat value for all case pools was calculated as X̅=NᵢPhatᵢ/ΣNᵢ where Nᵢ is the number of alleles (2× the number of people) in each case pool and the weighted p-hat value for all control pools were calculated as Y̅=ΣLⱼPhatⱼ/ΣLⱼ where Lᵢ is the number of alleles in each control pool. The estimate of delta-phat, the difference in allele frequency between cases and controls was X̅- Y̅ and the significance of delta-phat was determined using a Student's t statistic where

$$t = \frac{\overline{X} - \overline{Y}}{\sqrt{{S}_{p} ( {( \sum {N}_{i} )}^{- 1} + {( \sum {L}_{i} )}^{- 1} )}}$$

where Sₚ is the pooled variance under the assumption that both case and control variances are equal. The variance of X̅ was calculated as S_{X}=(¹/₈₋₁)ΣNᵢ(Phatᵢ− X̅)² and similarly the variance of Y̅ was calculated as S_{Y}=(¹/₈₋₁)ΣLⱼ(Phatⱼ− Y̅)². The Student's t test was performed with 8+8−2=14 degrees of freedom. Single Marker P values were calculated for all markers within the genome wide scan map.

### Combined P Values

In addition to single marker P values, combined P values across multi-marker sliding windows were calculated after the method of Fisher (Statistical Methods for Research Workers, 14^{th} edition Hafner Press N.Y. 1970 pp 99-100) where χ²=lnPᵢ with 2 k degrees of freedom where Pᵢ is the P value for association of each of k markers within the sliding window. The combined P values identify regions of multiple single marker associations. However, Fisher's method assumes independence of association for each marker which is known not to be the case because of strong LD between adjacent markers. Therefore the magnitude of the combined P value was affected both by LD and association and these cannot be taken at face value. The combined P values were best used as a means to identify regions of multi-marker association which can then be assessed on the basis of the magnitude of single marker association.

### Permutation Test for Exact P Values

There were 10 case and 10 control pools, each giving an estimate of p-hat. Therefore there are 20!(20 factorial)/10!×10!=184756 ways to group the pools into 2 groups of 10. The POOLEX exact test calculates the mean difference in phat for single markers between the two groups for all of these possible arrangements of the pools into two groups as well as the combined P values for 5, 9, and 15 marker windows for all combinations. These values are then arranged in order in each case and the relative rank among all combinations of the actually observed combination for real cases and controls is determined. The exact P value for the observed case and control combination is calculated as:

No. of combinations with a value greater than or equal to the actual observed value 184756 total combinations

For example, if the actually observed combined P value for a 5 marker window corresponding to the actual cases and controls combination, were the largest combined P value of all 184756 possible combinations of 2 groups of 10, then the exact probability (P value) for observing this event by chance given no difference between cases and controls would be 1/184756=0.000005412.

### Example 4

### Confirmatory Mapping and Fine Mapping

42 of the 47 top regions identified as being associated with longevity by the GWS are further analyzed by confirmatory mapping (genotyping all cases and controls samples individually) followed by fine mapping using a denser set of markers, in order to validate and/or refine the signal. Both confirmatory and fine mapping are carried out using the Illumina BeadStation 500GX SNP genotyping platform. Alleles are genotyped using an allele-specific elongation assay that involves ligation to a locus-specific oligonucleotide. The assay is performed directly on genomic DNA at a highly multiplex level and the products are amplified using universal oligonucleotides. For each candidate region, a set of SNP markers is selected with an average inter-marker distance varying with the mean extent of LD throughout the region as determined by delta-M (Δ_{M}), where M is the number of markers present in the 300 kb window centered at each reference marker is defined as the square root of the average r² or Δ²ᵢⱼ measures of LD between all ({M(M−1)}/2) pairwise comparisons of all (M) markers within the 300 kb window (Dawson et al., 2002). This produces an average multi-marker measure of LD analogous to Hill's Δ statistic for two marker LD. Regions with a signal harboring a high −Log₁₀ P value and with mean delta-M of 0.3 or below are mapped with a target density of one marker per 10 kb. Regions showing a signal with a high −Log₁₀ P value and with mean delta-M between 0.3 and 0.35 as well as selected regions with a signal with a lower −Log₁₀ P value and a delta-M value below 0.35 are mapped with a target density of one marker per 10-20 kb. The principle is that low-LD regions will be mapped at a higher SNP density. Selected regions with a delta-M value above 0.35 are mapped with a density of one marker per 20-30 kb, including the markers used in the GWS. The selected regions are delimited by the location where the LDSTATS −Log₁₀ P values reach the background level. The cohort consists of 615 cases and 615 controls (as used for the GWS).

Table 3 lists the fine mapping SNPs for the 42 confirmed regions and their respective p values using 615 cases and 615 controls trios and two analysis methods: LDSTATS(v4.0) and SingleType. For each region that was associated with longevity in the fine mapping analyses, we report in Table 7 the allele frequencies and the relative risk (RR) for the haplotypes contributing to the best signal at each SNP in the region. The best signal at a given location was determined by comparing the significance (p-value) of the association with longevity for multiple window sizes, and selecting the most significant window. For a given window size at a given location, the association with longevity was evaluated by comparing the overall distribution of haplotypes in the cases with the overall distribution of haplotypes in the controls. Haplotypes with a relative risk greater than one increase the risk of longevity while haplotypes with a relative risk less than one are protective and decrease longevity.

### Haplotype Association Analysis

Haplotype association analysis was performed using the program LDSTATS. LDSTATS tests for association of haplotypes with the disease phenotype. The algorithm LDSTATS (v4.0) defines haplotypes using multi-marker windows that advance across the marker map in one-marker increments. Windows can contain any odd number of markers specified as a parameter of the algorithm. Other marker windows can also be used. At each position the frequency of haplotypes in cases and controls was calculated and a chi-square statistic was calculated from case control frequency tables. LDSTATS v4.0 calculates significance of chi-square values using a permutation test in which case-control status is randomly permuted until 350 permuted chi-square values are observed that are greater than or equal to chi-square value of the actual data. The P value is then calculated as 350/the number of permutations required.

### Singletype Analysis

The SINGLETYPE algorithm assesses the significance of case-control association for single markers using the genotype data from the laboratory as input in contrast to LDSTATS single marker window analyses, in which case-control alleles for single markers from estimated haplotypes are used as input. SINGLETYPE calculates P values for association for both alleles, 1 and 2, as well as for genotypes, 11, 12, and 22, and plots these as −log₁₀ P values for significance of association against marker position.

### Example 5

### Gene Identification and Characterization

A series of gene characterization steps was performed for each candidate region described in Table 1. Any gene or EST mapping to the interval based on public map data or proprietary map data was considered as a candidate longevity gene. The approach used to identify all genes located in the critical regions is described below.

### Public Gene Mining

Once regions were identified using the analyses described above, a series of public data mining efforts were undertaken, with the aim of identifying all genes located within the critical intervals as well as their respective structural elements (i.e., promoters and other regulatory elements, UTRs, exons and splice sites). The initial analysis relied on annotation information stored in public databases (e.g. NCBI, UCSC Genome Bioinformatics, Entrez Human Genome Browser, OMIM—see below for database URL information).

Database URLs

| Name | URL |
|---|---|
| Biocarta | http://www.biocarta.com/ |
| BioCyc | http://www.biocyc.org/ |
| Bimolecular Interaction Network | http://bind.ca/ |
| Database (BIND) | |
| Database of Interacting Proteins | http://dip.doe-mbi.ucla.edu/ |
| Gene Expression Omnibus | http://www.ncbi.nlm.nih.gov/geo/ |
| Human Genome Browser | http://www.ensembl.org/Homo_sapiens/ |
| Intercom | http://interdom.lit.org.sg/help/term.php |
| Kyoto Encyclopedia of Genes and | http://www.genome.jp/kegg/ |
| Genomes (KEGG) | |
| Molecular Interactions Database | http://mint.bio.uniroma2.it/mint/ |
| (MINT) | |
| National Center for Biotechnology | http://www.ncbi.nlm.nih.gov/ |
| Information (NCBI) | |
| Online Mendelian Inheritance in | http://www.ncbi.nlm.nih.gov/entrez/query.fcgi?db=OMIM |
| Man (OMIM) | |
| OmniViz | http://www.omniviz.com/applications/omni_viz.htm |
| Pathway Enterprise | http://www.omniviz.com/applications/pathways.htm |
| Reactome | http://www.reactome.org/ |
| Transpath | http://www.biobase.de/pages/products/transpath.html |
| UCSC Genome Bioinformatics | http://genome.ucsc.edu/index.html?org=Human |
| UniGene | http://www.ncbi.nlm.nih.gov/entrez/query.fcgi?db=unigene |

For some genes the available public annotation was extensive, whereas for others very little was known about a gene's function. Customized analysis was therefore performed to characterize genes that corresponded to this latter class. Importantly, the presence of rare splice variants and artifactual ESTs was carefully evaluated. Subsequent cluster analysis of novel ESTs provided an indication of additional gene content in some cases. The resulting clusters were graphically displayed against the genomic sequence, providing indications of separate clusters that may contribute to the same gene, thereby facilitating development of confirmatory experiments in the laboratory. While much of this information was available in the public domain, the customized analysis performed revealed additional information not immediately apparent from the public genome browsers.

A unique consensus sequence was constructed for each splice variant and a trained reviewer assessed each alignment. This assessment included examination of all putative splice junctions for consensus splice donor/acceptor sequences, putative start codons, consensus Kozak sequences and upstream in-frame stops, and the location of polyadenylation signals. In addition, conserved noncoding sequences (CNSs) that could potentially be involved in regulatory functions were included as important information for each gene. The genomic reference and exon sequences were then archived for future reference. A master assembly that included all splice variants, exons and the genomic structure was used in subsequent analyses (i.e., analysis of polymorphisms).

An important component of these efforts was the ability to visualize and store the results of the data mining efforts. A customized version of the highly versatile genome browser GBrowse (http://www.gmod.org/) was implemented in order to permit the visualization of several types of information against the corresponding genomic sequence. In addition, the results of the statistical analyses were plotted against the genomic interval, thereby greatly facilitating focused analysis of gene content.

### Computational Analysis of Genes and GeneMaps

In order to assist in the prioritization of candidate genes for which minimal annotation existed, a series of computational analyses were performed that included basic BLAST searches and alignments to identify related genes. In some cases this provided an indication of potential function. In addition, protein domains and motifs were identified that further assisted in the understanding of potential function, as well as predicted cellular localization.

A comprehensive review of the public literature was also performed in order to facilitate identification of information regarding the potential role of candidate genes in the pathophysiology of longevity trait and/or age-associated disorders. In addition to the standard review of the literature, public resources (Medline and other online databases) were also mined for information regarding the involvement of candidate genes in specific signaling pathways. The Ingenuity Pathway Analysis System was also used to generate protein interaction networks. A variety of pathway and yeast two hybrid databases were mined for information regarding protein-protein interactions. These included BIND, MINT, DIP, Interdom, and Reactome, among others. By identifying homologues of genes in the longevity candidate regions and exploring whether interacting proteins had been identified already, knowledge regarding the GeneMaps for longevity was advanced. The pathway information gained from the use of these resources was also integrated with the literature review efforts, as described above.

### 3. Expression Studies

In order to determine the expression patterns for genes, relevant information was first extracted from public databases. The UniGene database, for example, contains information regarding the tissue source for ESTs and cDNAs contributing to individual clusters. This information was extracted and summarized to provide an indication in which tissues the gene was expressed. Particular emphasis was placed on annotating the tissue source for bona fide ESTs, since many ESTs mapped to Unigene clusters are artifactual. In addition, SAGE and microarray data, also curated at NCBI (Gene Expression Omnibus), provided information on expression profiles for individual genes. Particular emphasis was placed on identifying genes that were expressed in tissues known to be involved in the pathophysiology of longevity trait and/or age-associated disorders.

### 4. Polymorphism Analysis

Polymorphisms identified in candidate genes, including those from the public domain as well as those identified by sequencing candidate genes and regions, are evaluated for potential function. Initially, polymorphisms are examined for potential impact upon encoded proteins. If the protein is a member of a gene family with reported 3-dimensional structural information, this information is used to predict the location of the polymorphism with respect to protein structure. This information provided insight into the potential role of polymorphisms in altering protein or ligand interactions, as well as suitability as a drug target. In a second phase of analysis we evaluate the potential role of polymorphisms in other biological phenomena, including regulation of transcription, splicing and mRNA stability, etc. There are many examples of the functional involvement of naturally occurring polymorphisms in these processes. As part of this analysis, polymorphisms located in promoter or other regulatory elements, canonical splice sites, exonic and intronic splice enhancers and repressors, conserved noncoding sequences and UTRs are localized.

### Example 6

### SNP and Polymorphism Discovery (SNPD)

Candidate genes and regions are selected for sequencing in order to identify all polymorphisms. In cases where the critical interval, identified by fine mapping, was relatively small (˜50 kb), the entire region, including all introns, is sequenced to identify polymorphisms. In situations where the region is large (>50 kb), candidate genes are prioritized for sequencing, and/or only functional gene elements (promoters, exons and splice sites) are sequenced.

The samples to be sequenced are selected according to which haplotypes contribute to the association signal observed in the region. The purpose is to select a set of samples that covered all the major haplotypes in the given region. Each major haplotype must be present in a few copies. The first step therefore consisted of determining the major haplotypes in the region to be sequenced.

Once a region is defined with the two boundary markers, all the markers used in fine mapping that are located within the region are used to determine the major haplotypes. Long haplotypes covering the whole region are thus inferred using the middle marker as an anchor. The results included two series of haplotype themes that define the major haplotypes, comparing the cases and the controls. This exercise is repeated using an anchor in the peripheral regions to ensure that major haplotype subsets that are not anchored at the original middle marker are not missed.

Once the major haplotypes are determined as described above, appropriate genomic DNA samples are selected such that each major haplotype and haplotype subset are represented in at least two to four copies.

The protocol includes the following steps, once a region is delimited:

### Primer Design

The design of the primers is performed using a proprietary primer design tool. A primer quality control is included in the primer design process. Primers that successfully passed the control quality process were synthesized by Integrated DNA Technologies (IDT). The sense and anti-sense oligos are separated such that the sense oligos are placed on one plate in the same position as their anti-sense counterparts are on another plate. Two additional plates are created from each storage plate, one for use in PCR and the other for sequencing. For PCR, the sense and anti-sense oligos of the same pair are combined in the same well to achieve a final concentration of 1.5 μM for each oligonucleotide.

### PCR Optimization

PCR conditions are optimized by testing a variety of conditions that included varying salt concentrations and temperatures, as well as including various additives. PCR products are checked for robust amplification and minimal background by agarose gel electrophoresis.

### PCR on Selected Samples

PCR products to be used for sequencing are amplified using the conditions chosen during optimization. The PCR products are purified free of salts, dNTPs and unincorporated primers by use of a MultiScreen PCR384 filter plate manufactured by Millipore. Following PCR, the amplicons are quantified by use of a lambda/Hind III standard curve. This is done to ensure that the quantity of PCR product required for sequencing had been generated. The raw data was measured against the standard curve data in Excel by use of a macro.

### Sequencing

Sequencing of PCR products is performed by DNA Landmarks using ABI 3730 capillary sequencing instruments.

### Sequence Analysis

The ABI Prism SeqScape software (Applied Biosystems) is used for SNP identification. The chromatogram trace files were imported into a SeqScape sequencing project and the base calling is automatically performed. Sequences are then aligned and compared to each other using the SeqScape program. The base calling is checked manually, base by base; editing was performed if needed.

### Example 7

### Ultra Fine Mapping (UFM)

Once polymorphisms are identified by sequencing efforts as described in Example 6, additional genotyping of all newly found polymorphisms is performed on the samples used in the fine mapping studies. Various types of genotyping assays may need to be utilized based on the type of polymorphism identified (i.e., SNP, indel, microsatellite). The assay type can be, but is not restricted to, Sentrix Assay Matrix on Illumina BeadStations, microsatellite on MegaBACE, SNP on ABI or Orchid. The frequencies of genotypes and haplotypes in cases and controls are analyzed in a similar manner as the GWS and fine mapping data. By examining all SNPs in a region, polymorphisms are identified that increase an individual's susceptibility to longevity. The goal of ultra-fine mapping is to identify the polymorphism that is most associated with disorder phenotype as part of the search for the actual DNA polymorphism that confers susceptibility to disorder. This statistical identification may need to be corroborated by functional studies.

### Example 8

### Confirmation of Candidate Regions and Genes in a General Population

The confirmation of any putative associations described in Example 7 is performed in an independent general population patient sample. These DNA samples consist of at least 400 male controls and 400 male patients with longevity.

All publications, patents and patent applications mentioned in the specification and reference list are herein incorporated by reference in their entirety for all purposes. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, or related fields are intended to be within the scope of the following claims.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular CloningA Laboratory Manual, 2nd Ed., ed. by Sambrook, Fritsch and Maniatis (Cold Spring Harbor Laboratory Press: 1989); DNA Cloning, Volumes I and H (D. N. Glover ed., 4); Oligonucleotide Synthesis (M. J. Gait ed., 1984); Mullis et al U.S. Pat. No. 4,683,195; Nucleic Acid Hybridization (B. D. Hames & S. J. Higgins eds. 1984); Transcription And Translation (B. D. Haines & S. J. Higgins eds. 1984); Culture Of Animal Cells (R. 1. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### REFERENCES

Alwine, J. C., D. J. Kemp, et al. (1977). “Method for detection of specific RNAs in agarose gels by transfer to diazobenzyloxymethyl-paper and hybridization with DNA probes.” Proc Natl Acad Sci USA 74(12): 5350-4.
Alwine, J. C., Reed, S. I., & Stark, G. R. (1977). Characterization of the autoregulation of simian virus 40 gene A. J Virol, 24(1), 22-27.
Anderson, W. F. (1992). “Human gene therapy.” Science 256(5058): 808-13.
Antonarakis, S. E. (1987). “Hemophilia A persistence and gene mutational vulnerability.” Hosp Pract (Off Ed) 22(12): 93-5, 99-102.
Antonarakis, S. E., H. Youssoufian, et al. (1987). “Molecular genetics of hemophilia A in man (factor VIII deficiency).” Mol Biol Med 4(2): 81-94.
Barany, F. (1991). “Genetic disease detection and DNA amplification using cloned thermostable ligase.” Proc Natl Acad Sci USA 88(1): 189-93.
Barre, F. X., S. Ait-Si-Ali, et al. (2000). “Unambiguous demonstration of triple-helix-directed gene modification.” Proc Natl Acad Sci USA 97(7): 3084-8.
Barzilai, N., I. Gabriely, et al. (2001). “Offspring of centenarians have a favorable lipid profile.” J Am Geriatr Soc 49(1): 76-9.
Beckman, K. B. and B. N. Ames (1999). “Endogenous oxidative damage of mtDNA.” Mutat Res 424(1-2): 51-8.
Bourgeois, S. and D. Labuda (2004). “Dynamic allele-specific oligonucleotide hybridization on solid support.” Anal Biochem 324(2): 309-11.
Cale, J. M., C. E. Shaw, et al. (1998). “Optimization of a reverse transcription-polymerase chain reaction (RT-PCR) mass assay for low-abundance mRNA.” Methods Mol Biol 105: 351-71.
Chen, X., L. Levine, et al. (1999). “Fluorescence polarization in homogeneous nucleic acid analysis.” Genome Res 9(5): 492-8.
Chien, C. T., P. L. Bartel, et al. (1991). “The two-hybrid system: a method to identify and clone genes for proteins that interact with a protein of interest.” Proc Natl Acad Sci USA 88(21): 9578-82.
Cohen, A. S., D. L. Smisek, et al. (1996). “Emerging technologies for sequencing antisense oligonucleotides: capillary electrophoresis and mass spectrometry.” Adv Chromatogr 36: 127-62.
Cotton, R. G., N. R. Rodrigues, et al. (1988). “Reactivity of cytosine and thymine in single-base-pair mismatches with hydroxylamine and osmium tetroxide and its application to the study of mutations.” Proc Natl Acad Sci USA 85(12): 4397-401.
Cronin, M. T., R. V. Fucini, et al. (1996). “Cystic fibrosis mutation detection by hybridization to light-generated DNA probe arrays.” Hum Mutat 7(3): 244-55.
Dean, F. B., S. Hosono, et al. (2002). “Comprehensive human genome amplification using multiple displacement amplification.” Proc Natl Acad Sci USA 99(8): 5261-6.
Dillon, N. (1993). “Regulating gene expression in gene therapy.” Trends Biotechnol 11(5): 167-73.
D'Mello N, P., A. M. Childress, et al. (1994). “Cloning and characterization of LAG1, a longevity-assurance gene in yeast.” J Biol Chem 269(22): 15451-9.
Drmanac, R., S. Drmanac, et al. (2002). “Sequencing by hybridization (SBH): advantages, achievements, and opportunities.” Adv Biochem Eng Biotechnol 77: 75-101.
Elez, R., A. Piiper, et al. (2000). “Polo-like kinase1, a new target for antisense tumor therapy.” Biochem Biophys Res Commun 269(2): 352-6.
Evans, B. E., K. E. Rittle, et al. (1987). “Design of nonpeptidal ligands for a peptide receptor: cholecystokinin antagonists.” J Med Chem 30(7): 1229-39.
Fackler, O. T. and B. M. Peterlin (2000). “Endocytic entry of HIV-1.” Curr Biol 10(16): 1005-8.
Fakhrai-Rad, H., J. Zheng, et al. (2004). “SNP discovery in pooled samples with mismatch repair detection.” Genome Res 14(7): 1404-12.
Faragher, R. G., I. R. Kill, et al. (1993). “The gene responsible for Werner syndrome may be a cell division “counting” gene.” Proc Natl Acad Sci USA 90(24): 12030-4.
Fields, S. and O, Song (1989). “A novel genetic system to detect protein-protein interactions.” Nature 340(6230): 245-6.
Frank-Kamenetskii, M. D. and S. M. Mirkin (1995). “Triplex DNA structures.” Annu Rev Biochem 64: 65-95.
Freeman, W. M., S. J. Walker, et al. (1999). “Quantitative RT-PCR: pitfalls and potential.” Biotechniques 26(1): 112-22, 124-5.
Gasparini, P., A. Bonizzato, et al. (1992). “Restriction site generating-polymerase chain reaction (RG-PCR) for the probeless detection of hidden genetic variation: application to the study of some common cystic fibrosis mutations.” Mol Cell Probes 6(1): 1-7.
Gibbs, R. A., P. N. Nguyen, et al. (1989). “Detection of single DNA base differences by competitive oligonucleotide priming.” Nucleic Acids Res 17(7): 2437-48.
Griffin, H. G. and A. M. Griffin (1993). “DNA sequencing. Recent innovations and future trends.” Appl Biochem Biotechnol 38(1-2): 147-59.
Grossman, P. D., W. Bloch, et al. (1994). “High-density multiplex detection of nucleic acid sequences: oligonucleotide ligation assay and sequence-coded separation.” Nucleic Acids Res 22(21): 4527-34.
Guatelli, J. C., K. M. Whitfield, et al. (1990). “Isothermal, in vitro amplification of nucleic acids by a multienzyme reaction modeled after retroviral replication.” Proc Natl Acad Sci USA 87(5): 1874-8.
Hafner, G. J., I. C. Yang, et al. (2001). “Isothermal amplification and multimerization of DNA by Bst DNA polymerase.” Biotechniques 30(4): 852-6, 858, 860 passim.
Hardenbol, P., J. Baner, et al. (2003). “Multiplexed genotyping with sequence-tagged molecular inversion probes.” Nat Biotechnol 21(6): 673-8. Epub 2003 May 5.
Hayashi, K. (1992). “PCR-SSCP: a method for detection of mutations.” Genet Anal Tech Appl 9(3): 73-9.
Helbock, H. J., K. B. Beckman, et al. (1999). “8-Hydroxydeoxyguanosine and 8-hydroxyguanine as biomarkers of oxidative DNA damage.” Methods Enzymol 300: 156-66.
Hermonat, P. L. and N. Muzyczka (1984). “Use of adeno-associated virus as a mammalian DNA cloning vector: transduction of neomycin resistance into mammalian tissue culture cells.” Proc Natl Acad Sci USA 81(20): 6466-70.
Hitt, R., Y. Young-Xu, et al. (1999). “Centenarians: the older you get, the healthier you have been.” Lancet 354(9179): 652.
Hogrefe, H. H., R. L. Mullinax, et al. (1993). “A bacteriophage lambda vector for the cloning and expression of immunoglobulin Fab fragments on the surface of filamentous phage.” Gene 128(1): 119-26.
Hoogendoorn, B., N. Norton, et al. (2000). “Cheap, accurate and rapid allele frequency estimation of single nucleotide polymorphisms by primer extension and DHPLC in DNA pools.” Hum Genet 107(5): 488-93.
Hsu, I. C., Q. Yang, et al. (1994). “Detection of DNA point mutations with DNA mismatch repair enzymes.” Carcinogenesis 15(8): 1657-62.
Jazwinski, S. M., J. B. Chen, et al. (1993). “A single gene change can extend yeast life span: the role of Ras in cellular senescence.” Adv Exp Med Biol 330: 45-53.
Jobs, M., W. M. Howell, et al. (2003). “DASH-2: flexible, low-cost, and high-throughput SNP genotyping by dynamic allele-specific hybridization on membrane arrays.” Genome Res 13(5): 916-24.
Kanazawa, N., M. Kurosaki, et al. (2004). “Regulation of hepatitis C virus replication by interferon regulatory factor 1.” J Virol 78(18): 9713-20.
Kanehisa, M. (1984). “Use of statistical criteria for screening potential homologies in nucleic acid sequences.” Nucleic Acids Res 12(1 Pt 1): 203-13.
Kohno, T., T. Otsuka, et al. (1995). “Identification of a novel phospholipase C family gene at chromosome 2q33 that is homozygously deleted in human small cell lung carcinoma.” Hum Mol Genet 4(4): 667-74.
Kohler, G. and C. Milstein (1992). “Continuous cultures of fused cells secreting antibody of predefined specificity. 1975.” Biotechnology 24: 524-6.
Kotin, R. M. (1994). “Prospects for the use of adeno-associated virus as a vector for human gene therapy.” Hum Gene Ther 5(7): 793-801.
Kozal, M. J., N. Shah, et al. (1996). “Extensive polymorphisms observed in HIV-1 clade B protease gene using high-density oligonucleotide arrays.” Nat Med 2(7): 753-9.
Krebs, S., D. Seichter, et al. (2001). “Genotyping of dinucleotide tandem repeats by MALDI mass spectrometry of ribozyme-cleaved RNA transcripts.” Nat Biotechnol 19(9): 877-80.
Kremer, E. J. and M. Perricaudet (1995). “Adenovirus and adeno-associated virus mediated gene transfer.” Br Med Bull 51(1): 31-44.
Kulinski, J., D. Besack, et al. (2000). “CEL I enzymatic mutation detection assay.” Biotechniques 29(1): 44-6, 48.
Law, S. W., K. J. Lackner, et al. (1986). “The molecular biology of human apoA-I, apoA-II, apoC-II and apoB.” Adv Exp Med Biol 201: 151-62.
Leppert, M., J. L. Breslow, et al. (1988). “Inference of a molecular defect of apolipoprotein B in hypobetalipoproteinemia by linkage analysis in a large kindred.” J Clin Invest 82(3): 847-51.
Livak, K. J., J. Marmaro, et al. (1995). “Towards fully automated genome-wide polymorphism screening.” Nat Genet 9(4): 341-2.
Lizardi, P. M., X. Huang, et al. (1998). “Mutation detection and single-molecule counting using isothermal rolling-circle amplification.” Nat Genet 19(3): 225-32.
Ljungquist, B., S. Berg, et al. (1998). “The effect of genetic factors for longevity: a comparison of identical and fraternal twins in the Swedish Twin Registry.” J Gerontol A Biol Sci Med Sci 53(6): M441-6.
Maiato, H., C. L. Rieder, et al. (2003). “How do kinetochores CLASP dynamic microtubules?” Cell Cycle 2(6): 511-4.
Mansfield, E. S., M. Vainer, et al. (1996). “Sensitivity, reproducibility, and accuracy in short tandem repeat genotyping using capillary array electrophoresis.” Genome Res 6(9): 893-903.
Maxam, A. M. and W. Gilbert (1977). “A new method for sequencing DNA.” Proc Natl Acad Sci USA 74(2): 560-4.
McGue, M., J. W. Vaupel, et al. (1993). “Longevity is moderately heritable in a sample of Danish twins born 1870-1880.” J Gerontol 48(6): B237-44.
Mehrabian, M., R. S. Sparkes, et al. (1986). “Human apolipoprotein B: chromosomal mapping and DNA polymorphisms of hepatic and intestinal species.” Somat Cell Mol Genet 12(3): 245-54.
Miller, A. D. (1992). “Human gene therapy comes of age.” Nature 357(6378): 455-60.
Miller, A. D., J. V. Garcia, et al. (1991). “Construction and properties of retrovirus packaging cells based on gibbon ape leukemia virus.” J Virol 65(5): 2220-4.
Mitani, K. and C. T. Caskey (1993). “Delivering therapeutic genes—matching approach and application.” Trends Biotechnol 11(5): 162-6.
Muzyczka, N. (1994). “Adeno-associated virus (AAV) vectors: will they work?” J Clin Invest 94(4): 1351.
Myers, R. M., Z. Larin, et al. (1985). “Detection of single base substitutions by ribonuclease cleavage at mismatches in RNA:DNA duplexes.” Science 230(4731): 1242-6.
Myers, R. M., N. Lumelsky, et al. (1985). “Detection of single base substitutions in total genomic DNA.” Nature 313(6002): 495-8.
Nabel, G. J. and P. L. Felgner (1993). “Direct gene transfer for immunotherapy and immunization.” Trends Biotechnol 11(5): 211-5.
Nagley, P. and Y. H. Wei (1998). “Ageing and mammalian mitochondrial genetics.” Trends Genet 14(12): 513-7.
Nathan, C. and M. U. Shiloh (2000). “Reactive oxygen and nitrogen intermediates in the relationship between mammalian hosts and microbial pathogens.” Proc Natl Acad Sci USA 97(16): 8841-8.
Niu, T., Z. S. Qin, et al. (2002). “Bayesian haplotype inference for multiple linked single-nucleotide polymorphisms.” Am J Hum Genet 70(1): 157-69.
Orita, M., H. Iwahana, et al. (1989). “Detection of polymorphisms of human DNA by gel electrophoresis as single-strand conformation polymorphisms.” Proc Natl Acad Sci USA 86(8): 2766-70.
Odley, A., H. S. Hahn, et al. (2004). “Regulation of cardiac contractility by Rab4-modulated beta2-adrenergic receptor recycling.” Proc Natl Acad Sci USA 101 (18): 7082-7. Epub 2004 Apr. 22.
Overgaard, M. T., H. B. Boldt, et al. (2001). “Pregnancy-associated plasma protein-A2 (PAPP-A2), a novel insulin-like growth factor-binding protein-5 proteinase.” J Biol Chem 276(24): 21849-53. Epub 2001 Mar. 22.
Perls, T. T., E. Bubrick, et al. (1998). “Siblings of centenarians live longer.” Lancet 351(9115): 1560
Permutt, M. A., Wasson, J. C., Suarez, B. K., Lin, J., Thomas, J., Meyer, J., Lewitzky, S., Rennich, J. S., Parker, A., DuPrat, L., Maruti, S., Chayen, S., & Glaser, B. (2001). A genome scan for type 2 diabetes susceptibility loci in a genetically isolated population. Diabetes, 50(3), 681-685.
Raap, A. K. (1998). “Advances in fluorescence in situ hybridization.” Mutat Res 400(1-2): 287-98.
Remick, D. G. (1995). “Applied molecular biology of sepsis.” J Crit Care 10(4): 198-212.
Ronaghi, M., M. Uhlen, et al. (1998). “A sequencing method based on real-time pyrophosphate.” Science 281(5375): 363, 365.
Rosenecker, J., K. H. Harms, et al. (1996). “Adenovirus infection in cystic fibrosis patients: implications for the use of adenoviral vectors for gene transfer.” Infection 24(1): 5-8.
Saiki, R. K., T. L. Bugawan, et al. (1986). “Analysis of enzymatically amplified beta-globin and HLA-DQ alpha DNA with allele-specific oligonucleotide probes.” Nature 324(6093): 163-6.
Saiki, R. K., P. S. Walsh, et al. (1989). “Genetic analysis of amplified DNA with immobilized sequence-specific oligonucleotide probes.” Proc Natl Acad Sci USA 86(16): 6230-4.
Saleeba, J. A. and R. G. Cotton (1993). “Chemical cleavage of mismatch to detect mutations.” Methods Enzymol 217: 286-95.
Samulski, R. J., L. S. Chang, et al. (1989). “Helper-free stocks of recombinant adeno-associated viruses: normal integration does not require viral gene expression.” J Virol 63(9): 3822-8.
Sanger, F., S. Nicklen, et al. (1977). “DNA sequencing with chain-terminating inhibitors.” Proc Natl Acad Sci USA 74(12): 5463-7.
Sauter, E. R., M. Herlyn, et al. (2000). “Prolonged response to antisense cyclin D1 in a human squamous cancer xenograft model.” Clin Cancer Res 6(2): 654-60.
Schachter, F., L. Faure-Delanef, et al. (1994). “Genetic associations with human longevity at the APOE and ACE loci.” Nat Genet 6(1): 29-32.
Simon, H., I. Fortsch, et al. (1999). “Triple helix formation inhibits DNA gyrase activity.” Antisense Nucleic Acid Drug Dev 9(6): 527-31.
Sommerfelt, M. A. and R. A. Weiss (1990). “Receptor interference groups of 20 retroviruses plating on human cells.” Virology 176(1): 58-69.
Sterman, D. H., J. Treat, et al. (1998). “Adenovirus-mediated herpes simplex virus thymidine kinase/ganciclovir gene therapy in patients with localized malignancy: results of a phase I clinical trial in malignant mesothelioma.” Hum Gene Ther 9(7): 1083-92.
Storm, N., B. Darnhofer-Patel, et al. (2003). “MALDI-TOF mass spectrometry-based SNP genotyping.” Methods Mol Biol 212: 241-62.
Tornillo, L., Carafa, V., Richter, J., Sauter, G., Moch, H., Minola, E., Gambacorta, M., Bianchi, L., Vecchione, R., & Terracciano, L. M. (2000). Marked genetic similarities between hepatitis B virus-positive and hepatitis C virus-positive hepatocellular carcinomas. J Pathol, 192(3), 307-312.
Tratschin, J. D., M. H. West, et al. (1984). “A human parvovirus, adeno-associated virus, as a eucaryotic vector: transient expression and encapsidation of the procaryotic gene for chloramphenicol acetyltransferase.” Mol Cell Biol 4(10): 2072-81.
Walker, J. M. (1998). Pathomechanics and classification of cartilage lesions, facilitation of repair. J Orthop Sports Phys Ther, 28(4), 216-231.
West, A. B., Lockhart, P. J., O'Farell, C., & Farrer, M. J. (2003). Identification of a novel gene linked to parkin via a bi-directional promoter. J Mol Biol, 326(1), 11-19.
West, M. H., J. P. Trempe, et al. (1987). “Gene expression in adeno-associated virus vectors: the effects of chimeric mRNA structure, helper virus, and adenovirus VA1 RNA.” Virology 160(1): 38-47.
Wilson, C., M. S. Reitz, et al. (1989). “Formation of infectious hybrid virions with gibbon ape leukemia virus and human T-cell leukemia virus retroviral envelope glycoproteins and the gag and pol proteins of Moloney murine leukemia virus.” J Virol 63(5): 2374-8.
Wu, L., T. Sun, et al. (2002). “Identification of a family of mastermind-like transcriptional coactivators for mammalian notch receptors.” Mol Cell Biol 22(21): 7688-700.
Yajnik, V., Paulding, C., Sordella, R., McClatchey, A. I., Saito, M., Wahrer, D. C., Reynolds, P., Bell, D. W., Lake, R., van den Heuvel, S., Settleman, J., & Haber, D. A. (2003). DOCK4, a GTPase activator, is disrupted during tumorigenesis. Cell, 112(5), 673-684.
Yang, B., Wen, X., Kodali, N. S., Oleykowski, C. A., Miller, C. G., Kulinski, J., Besack, D., Yeung, J. A., Kowalski, D., & Yeung, A. T. (2000). Purification, cloning, and characterization of the CEL I nuclease. Biochemistry, 39(13), 3533-3541
Yu, M., E. Poeschla, et al. (1994). “Progress towards gene therapy for HIV infection.” Gene Ther 1(1): 13-26.

### Books

Abbas A K, Litchman A H. Cellular and Molecular Immunology. Philadelphia: Saunders; 1991. 417 p.
Austen B M and. Westwood O M R. Protein Targeting and Secretion. Oxford: IRL Press; 1991. 85 p.
Bishop M J, editor. Guide to Human Genome Computing, 2d ed. San Diego: Academic Press; 1998. 306 p.
Cowell I G, Austin C A, editors. DNA Library Protocols. Methods in Molecular Biology. Vol. 69 Totowa, N.J.:Humana Press; 1997. 321 p.
Freshney R I, editor. Animal Cell Culture: A Practical Approach. Oxford: IRL Press; 1986.
Freshney R I. Culture Of Animal Cells: A Manual of Basic Technique. New York: AR Liss; 1987. 397 p.
Glover D M, editor. DNA Cloning: A Practical Approach. Vols 1 & 2. Oxford; Washington: IRL Press; 1985.
Gribskov M, Devereux J, editors. Sequence Analysis Primer. Oxford University Press; 1994. 296 p.
Griffin A M, Griffin H G, editors. Computer Analysis of Sequence Data, Part 1. Totowa, N.J. Humana Press; 1994. 392 p.
Hames B D, Higgins S J, editors. Nucleic Acid Hybridization: A Practical Approach. Oxford: IRL Press; 1985. 245 p.
Hames B D, Higgins S J, editors. Transcription and Translation: A Practical Approach. Oxford: IRL Press; 1984. 328 p.
Harlow Ed, Lane D. Antibodies: A Laboratory Manual. New York: Cold Spring Harbor Laboratory; 1988. 726 p.
Heinje G. von. Sequence Analysis in Molecular Biology. San Diego: Academic Press; 1987. 188 p.
Hogan B, Costantini F, Lacy E, editors. Manipulating the Mouse Embryo: A Laboratory Manual. New York: Cold Spring Harbor Laboratory Press; 1986. 332 p.
Huber B E, Carr B I. Molecular and Immunologic Approaches. Mt. Kisco, N.Y.: Futura Publishing Co; 1994.
Jones J. Amino Acid and Peptide Synthesis. Oxford; New York: Oxford Science Publications; 1992. 86 p.
Kaufman P B, William W, Donghern K, editors. Handbook of Molecular and Cellular Methods in Biology and Medicine. Boca Raton: CRC Press; 1995. 484 p.
Lesk A M, editor. Computational Molecular Biology: sources and methods for sequence analysis. New York: Oxford University Press; 1988. 254 p.
Male D, Cooke A, Owen M, Trowsdale J, Champion B, editors. Advanced Immunology. 3rd ed. London; Baltimore: Mosby; 1996. 273 p.
McPherson M J, editor. Directed Mutagenesis: A Practical Approach. New York: IRL Press; 1991. 257 p.
McPherson M J, Quirke P, Taylor J R, editors. PCR: A Practical Approach. Oxford; New York: IRL Press; 1991. 253 p.
Miller J H, Calos M P, editors. Gene Transfer Vectors for Mammalian Cells. New York: Cold Spring Harbor Laboratory Press; 1987. 169 p.
Miller J H, Calos M P, editors. Gene Transfer Vectors For Mammalian Cells. New York: Cold Spring Harbor Laboratory; 1987. 169 p.
Pawlowitzki I H, Edwards J H, Thompson E A, editors. Genetic Mapping of Disease Genes. Academic Press London; 1997. 288 p.
Perbal B V. A Practical Guide to Molecular Cloning. 1st ed. New York: Wiley Interscience Publication; 1984. 554 p.
Perbal B V. A Practical Guide To Molecular Cloning. New York: Wiley; 1984. 554 p.
Peruski L F, Peruski A H. The Internet and the New Biology. Tools for Genomic and Molecular Research. Washington, D.C.: American Society for Microbiology Press; 1997.
Sambrook J. Molecular Cloning: A Laboratory Manual. 2nd ed. 3 vols. New York: Cold Spring Harbor Laboratory Press; 1989.
Sell S. Immunology, Immunopathology & Immunity. 5th ed. Stamford, Conn.: Appleton & Lange; 1996. 1014 p.
Smith D W, editor. Biocomputing. Informatics and Genome Projects, New York: Academic Press; 1993. 336 p.
Stites D P, Terr A T, editors. Basic and Clinical Immunology. 7th ed. Norwalk, Conn.: Appleton & Lange; 1991. 870 p.
Walker J M. Protein Protocols on CD-ROM, Humana Press, Totowa, N.J.
Weir D M, Herzenberg L A, Blackwell C, editors. Handbook Of Experimental Immunology. 4 vols. Oxford: Blackwell; 1986.
Woodward J. Immobilized Cells And Enzymes: A Practical Approach. Oxford: IRL Press; 1986.
Wu R, Grossman L, editors. Methods in Enzymology: Recombinant DNA Part E. Vol. 154. Amsterdam: Elsevier Science; 1987. 576 p.
Wu R, Grossman L, editors. Methods in Enzymology: Recombinant DNA Part F. Vol. 155. Amsterdam: Elsevier Science; 1987. 628 p.

### Patent

WO 30/42661A2
WO 1997US0005216
PCT/US94/05700
WO 0181628A1
WO 94/16101
WO 93/24641
U.S. Pat. No. 60/335,068
U.S. Pat. No. 5,869,242
U.S. Pat. No. 6,090,606
U.S. Pat. No. 5,888,819
U.S. Pat. No. 5,459,039
U.S. Pat. No. 5,498,531
U.S. Pat. No. 5,807,718
U.S. Pat. No. 4,683,195
U.S. Pat. No. 4,683,202
U.S. Pat. No. 4,797,368
U.S. Pat. No. 6,090,543
U.S. Pat. No. 6,107,065
U.S. Pat. No. 4,952,501.
U.S. 20040009479A1
U.S. Pat. No. 5,315,000
U.S. Pat. No. 5,585,089
U.S. Pat. No. 6,090,606
U.S. Pat. No. 5,869,242
U.S. Pat. No. 6,479,244
U.S. Pat. No. 4,797,368
U.S. Pat. No. 5,173,414

TABLE 1
List of Longevity candidate regions identified from the
genome wide scan and Fine Mapping association analyses. The first
column denotes the region identifier. The second and third columns
correspond to the chromosome and cytogenetic band, respectively.
The fourth and fifth columns correspond to the chromosomal start
and end coordinates of the NCBI genome assembly derived from
build 35 (B35).

| Region | Chromosome | Cytogenetic Band | B35 Start | B35 End |
|---|---|---|---|---|
| 1 | 1 | 1q23-q25 | 172922500 | 173790737 |
| 2 | 1 | 1q42-q43 | 225466378 | 225987741 |
| 3 | 1 | 1q42.13-q43 | 227029796 | 227560738 |
| 4 | 1 | 1q41-q44 | 236154391 | 236644953 |
| 5 | 2 | 2p24-p23 | 20891681 | 21414379 |
| 6 | 2 | 2p16.3 | 49817753 | 50101824 |
| 7 | 2 | 2p16.3 | 50101825 | 51414723 |
| 8 | 2 | 2q14.2-q14.3 | 121573870 | 122352090 |
| 9 | 2 | 2q14.3 | 127494872 | 128057833 |
| 10 | 2 | 2q23-q24 | 162067733 | 163003491 |
| 11 | 2 | 2q33 | 198533144 | 199078721 |
| 12 | 3 | 3p26.1-p25.1 | 6629288 | 8005030 |
| 13 | 3 | 3p21.33 | 32775326 | 33350489 |
| 14 | 3 | 3p21.3 | 39582260 | 40686361 |
| 15 | 4 | 4q21 | 77038761 | 77559991 |
| 16 | 4 | 4q23 | 100854365 | 101421476 |
| 17 | 4 | 4q28.1 | 126424833 | 127003431 |
| 18 | 4 | 4q31.21 | 143068047 | 143957383 |
| 19 | 6 | 6p22.1 | 27042873 | 27452810 |
| 20 | 6 | 6q25 | 152286110 | 153283388 |
| 21 | 7 | 7p14.3 | 30326902 | 30949204 |
| 22 | 7 | 7p14 | 32696611 | 33661735 |
| 23 | 7 | 7q31.3 | 127239803 | 127721306 |
| 24 | 7 | 7q35-q36 | 145001785 | 147803868 |
| 25 | 7 | 7q36.1 | 149454345 | 149955253 |
| 26 | 7 | 7q36.1 | 150075668 | 150561177 |
| 27 | 7 | 7q36 | 156602277 | 158116469 |
| 28 | 9 | 9p21.2 | 26700092 | 27297163 |
| 29 | 9 | 9q21.11 | 70132969 | 70960548 |
| 30 | 9 | 9q22.31 | 93175798 | 93764675 |
| 31 | 9 | 9q31.3-q32 | 110265356 | 110882740 |
| 32 | 10 | 10p12.31 | 19899999 | 20848770 |
| 33 | 10 | 10q23-q24 | 99966729 | 101219517 |
| 34 | 11 | 11p15.2 | 11697651 | 12231441 |
| 35 | 11 | 11p15.1 | 19443295 | 20335244 |
| 36 | 11 | 11q21 | 95101359 | 95908847 |
| 37 | 12 | 12q13 | 56066088 | 56635863 |
| 38 | 12 | 12q21.31 | 81345981 | 82268637 |
| 39 | 14 | 14q21.3 | 49608345 | 50174491 |
| 40 | 14 | 14q31 | 80247029 | 80930316 |
| 41 | 16 | 16q12.1 | 46450036 | 46985312 |
| 42 | 16 | 16q22-q23 | 73578487 | 74088978 |
| 43 | 16 | 16q23.3-q24.1 | 76445659 | 78049764 |
| 44 | 18 | 18q11.2 | 22503963 | 23258373 |
| 45 | 18 | 18q22-q23 | 61324317 | 61945465 |
| 46 | 22 | 22q11.21 | 16451100 | 17003269 |
| 47 | X | Xq13.3 | 80030757 | 80606131 |

TABLE 2

Longevity genome wide association study results in the Quebec Founder Population (QFP). SNP markers
found to be associated with longevity from the analysis of genome wide scan (GWS) data. Columns
include: Region ID; Chromosome; Build 35 location in base pairs (bp); rs#, dbSNP data base
(NCBI) reference number; Sequence ID, unique numerical identifier for this patent application;
Sequence, 21 bp of sequence covering 10 base pair of unique sequence flanking either side of central
polymorphic SNP; −log10 P values for GWS, −log10 of the P value for statistical significance
from the GWS for single SNP markers (both T test and Permutation test p-values are displayed; see
Example section) and for the most highly associated multi-marker haplotypes centered at the
reference marker and defined by the sliding windows of specified sizes (W05, W09 and W15).

Poolex

| | | | | | | | | | | |
|---|---|---|---|---|---|---|---|---|---|---|
| | | | | | | Single | Single Marker | | | |
| | | | | | | Marker | (Permutation | | | |
| Region ID | Chr | B35 Position | RS# | Seq ID | Flanking Sequence | (T test) | test) | W05 | W09 | W15 |

| | | | | | | | | | | |
|---|---|---|---|---|---|---|---|---|---|---|
| 1 | 1 | 172922500 | 12758275 | 4805 | GCAGTTTACTRATTCTTAGAT | 1.646 | 1.581 | 1.481 | 1.111 | 1.019 |
| 1 | 1 | 172951095 | 1553770 | 4806 | AAATGCTCAGYCTGTCTAATT | 1.216 | 1.269 | 1.377 | 1.148 | 1.035 |
| 1 | 1 | 172953249 | 1858537 | 4807 | GGGTCCTGGAWTGATGGTGTA | 1.153 | 1.099 | 1.170 | 0.925 | 0.952 |
| 1 | 1 | 172969990 | 352333 | 4808 | GTGCATCTGARAATAGAGGCT | 0.063 | 0.068 | 0.788 | 1.123 | 0.923 |
| 1 | 1 | 172980523 | — | 4809 | CCTGCTTTATRAAGACTATAT | 0.027 | 0.028 | 0.409 | 1.010 | 0.914 |
| 1 | 1 | 173002087 | 181280 | 4810 | CAAAAATACAWGTGTTTTTCA | 0.754 | 0.699 | 0.323 | 0.599 | 0.840 |
| 1 | 1 | 173006422 | 1503120 | 4811 | ATAGAGGGGTYATTTGCAAGT | 0.236 | 0.225 | 0.311 | 0.421 | 0.843 |
| 1 | 1 | 173016407 | 2504511 | 4812 | GTCTAGTGTCRCTTGGCTGTT | 0.825 | 0.856 | 0.398 | 0.265 | 0.787 |
| 1 | 1 | 173024350 | — | 4813 | CAAGTCAAACMGATTTTAAGC | 0.113 | 0.111 | 0.347 | 0.368 | 0.664 |
| 1 | 1 | 173030154 | 2455729 | 4814 | GATCGACATAMGTCTGCACAC | 0.202 | 0.204 | 0.458 | 0.465 | 0.835 |
| 1 | 1 | 173037068 | 2455727 | 4815 | CAAAGTGAGARTAGAATGAGA | 0.577 | 0.575 | 0.298 | 0.346 | 0.863 |
| 1 | 1 | 173049316 | 2455760 | 4816 | GACTGTGCTGSATTAACCAAT | 0.450 | 0.454 | 0.406 | 0.563 | 0.859 |
| 1 | 1 | 173067383 | 1354286 | 4817 | GAAAACTATAYATCATTGTAA | 0.590 | 0.550 | 0.408 | 0.856 | 0.947 |
| 1 | 1 | 173077283 | 12139848 | 4818 | CCCTGAGGAARAAAAGAAGTA | 0.410 | 0.404 | 0.588 | 1.148 | 0.920 |
| 1 | 1 | 173085783 | 1503124 | 4819 | GCTTGGTCCARTGTGTTTTGG | 0.242 | 0.237 | 1.316 | 1.144 | 0.927 |
| 1 | 1 | 173119991 | 2455725 | 4820 | ACAGGGTTTTSTTGCATAGAC | 1.016 | 1.023 | 1.505 | 1.177 | 0.724 |
| 1 | 1 | 173134901 | 1503116 | 4821 | TGTGAAAAGARCCTACTGTTC | 2.096 | 2.074 | 1.360 | 1.268 | 1.032 |
| 1 | 1 | 173143278 | 4652175 | 4822 | TTTGAGGCAGRATTTTATAAA | 1.293 | 1.312 | 1.588 | 1.148 | 1.586 |
| 1 | 1 | 173146438 | 4650943 | 4823 | TTGTAGAATTRTGACTAGTCT | 0.204 | 0.202 | 1.665 | 1.015 | 1.486 |
| 1 | 1 | 173157471 | 6425384 | 4824 | CTTAAAATGGRTATTTTTACT | 0.000 | 0.000 | 0.569 | 1.539 | 1.449 |
| 1 | 1 | 173164621 | — | 4825 | ATCCTCTGCASACACCTTCTC | 0.000 | 0.000 | 0.025 | 2.137 | 1.387 |
| 1 | 1 | 173171836 | 12742614 | 4826 | AGGTGATAGCYTGTGATATGC | 0.143 | 0.139 | 0.552 | 1.318 | 1.503 |
| 1 | 1 | 173177355 | — | 4827 | AATCTGTCTGSCACGCCTGCC | 0.031 | 0.028 | 1.454 | 0.776 | 1.851 |
| 1 | 1 | 173188880 | 6676641 | 4828 | TGCATGATATKGTTGTTTCTC | 1.511 | 1.420 | 1.185 | 0.872 | 1.813 |
| 1 | 1 | 173194705 | 1569639 | 4829 | CTTAGTTAGAKAATTTTTTTG | 2.272 | 2.155 | 1.118 | 0.951 | 1.433 |
| 1 | 1 | 173204977 | 10157031 | 4830 | CTAAGCAGCARTTTTTGAAAA | 0.011 | 0.011 | 1.340 | 1.264 | 1.168 |
| 1 | 1 | 173219357 | 10489473 | 4831 | TAATGCCTTTMAGTGCAGTTA | 0.088 | 0.088 | 0.952 | 1.445 | 1.080 |
| 1 | 1 | 173227979 | 6692448 | 4832 | GATATTGTATYGAAGAAGAAC | 0.404 | 0.395 | 0.508 | 1.651 | 0.990 |
| 1 | 1 | 173235591 | 7530550 | 4833 | GCTTCAACTGSAAGTATATAG | 0.710 | 0.769 | 0.909 | 1.298 | 0.905 |
| 1 | 1 | 173245194 | 2206510 | 4834 | GATTTGGAAAYACTGATGGCC | 1.288 | 1.230 | 1.331 | 0.655 | 1.057 |
| 1 | 1 | 173256851 | 2294654 | 4835 | AAGGACTAAARTACAGCAAGA | 0.866 | 0.857 | 1.266 | 0.682 | 1.188 |
| 1 | 1 | 173262289 | 1569638 | 4836 | CAGTTTGCTARATTGTAAAAT | 0.825 | 0.861 | 0.930 | 0.693 | 0.913 |
| 1 | 1 | 173268546 | 10913204 | 4837 | TACTGAGAGTRACTGGTCCTG | 0.736 | 0.722 | 0.543 | 0.848 | 0.498 |
| 1 | 1 | 173274944 | 10798461 | 4838 | ACAAGGCAGGWTTTGCCAGCT | 0.050 | 0.048 | 0.270 | 0.772 | 0.533 |
| 1 | 1 | 173280130 | 10489475 | 4839 | GTTGTATGCTSTTCCCATTAG | 0.080 | 0.082 | 0.244 | 0.476 | 0.913 |
| 1 | 1 | 173298693 | 16849993 | 4840 | CTCCTCAGAGYTTCCCTTTAT | 0.093 | 0.092 | 0.123 | 0.353 | 0.958 |
| 1 | 1 | 173303088 | 240104 | 4841 | TTGCCCATGGYACACTCCCCT | 0.875 | 0.869 | 0.195 | 0.163 | 0.793 |
| 1 | 1 | 173314633 | 10489479 | 4842 | GTAATGTGACYTCCAGGAAAT | 0.000 | 0.000 | 0.350 | 0.363 | 0.558 |
| 1 | 1 | 173328155 | — | 4843 | AAATTCTAGAYAAGAATCTGA | 0.284 | 0.285 | 0.370 | 0.509 | 0.460 |
| 1 | 1 | 173339083 | 6670258 | 4844 | CAAACACAATRCACATTAGAA | 0.459 | 0.457 | 0.718 | 0.556 | 0.323 |
| 1 | 1 | 173353855 | 6689901 | 4845 | TTAGTCACTTWGTCTGTTACT | 0.151 | 0.146 | 0.718 | 0.671 | 0.344 |
| 1 | 1 | 173364672 | — | 4846 | TTGTCTTTCTWAGTATCTGTA | 1.559 | 1.512 | 0.688 | 0.486 | 0.397 |
| 1 | 1 | 173370788 | 953541 | 4847 | GACGCTGTGAWAGGAACATTC | 0.000 | 0.000 | 0.643 | 0.410 | 0.524 |
| 1 | 1 | 173378657 | 17546000 | 4848 | CCATAGATAGRATAATGAAAA | 0.218 | 0.218 | 0.772 | 0.581 | 0.510 |
| 1 | 1 | 173385960 | 10494501 | 4849 | CAGACTGCACRTAGAATAATA | 0.405 | 0.401 | 0.190 | 0.532 | 0.857 |
| 1 | 1 | 173391904 | 726252 | 4850 | CTGTCTCTCAYGCCTTCCTTG | 0.355 | 0.361 | 0.341 | 0.678 | 1.087 |
| 1 | 1 | 173403942 | 791030 | 4851 | TGAACAAATGRCCTATTCTGC | 0.201 | 0.212 | 0.364 | 0.248 | 1.430 |
| 1 | 1 | 173409624 | 17354671 | 4852 | TGAACATGAARAAAAGGAAAT | 0.857 | 0.851 | 0.452 | 0.749 | 1.994 |
| 1 | 1 | 173424493 | 1995651 | 4853 | CCCAAAAAAAWTTCTATCATT | 0.348 | 0.319 | 0.308 | 1.167 | 3.026 |
| 1 | 1 | 173445270 | 10913246 | 4854 | GACTCAGTAGSATTTTACCTT | 0.656 | 0.630 | 1.205 | 1.437 | 2.702 |
| 1 | 1 | 173455106 | 10494502 | 4855 | TAGTGACTTAYCATAGTTCCT | 0.037 | 0.036 | 1.361 | 1.999 | 2.766 |
| 1 | 1 | 173462360 | 17352504 | 4856 | ACTATTAGATRTGCTATGTTT | 2.261 | 2.177 | 1.735 | 2.812 | 2.955 |
| 1 | 1 | 173469910 | 1325601 | 4857 | CAGAAACTGTKTAGAGCAGGC | 1.461 | 1.398 | 2.220 | 2.840 | 2.850 |
| 1 | 1 | 173475519 | 1325599 | 4858 | TTATTTCCCCRCATGAAAATG | 1.434 | 1.360 | 3.143 | 2.892 | 3.454 |
| 1 | 1 | 173481447 | 10913248 | 4859 | TGGCAGCTCTSTCAGGTCAGT | 2.400 | 2.348 | 3.044 | 3.065 | 3.198 |
| 1 | 1 | 173491681 | 10913254 | 4860 | TTTAAGCAGAMAGGCAAAAAC | 4.146 | 3.804 | 3.289 | 3.039 | 3.198 |
| 1 | 1 | 173503616 | 760939 | 4861 | TATTCTCTTGRCATAAGGATT | 0.618 | 0.587 | 3.542 | 3.748 | 3.130 |
| 1 | 1 | 173510267 | 10913257 | 4862 | CTTCTCTGTTKTCTTGGACCT | 1.061 | 1.032 | 2.688 | 3.526 | 3.369 |
| 1 | 1 | 173522159 | 10732997 | 4863 | ATTATTTGAARTAGATTTGAA | 0.604 | 0.590 | 2.231 | 3.496 | 3.262 |
| 1 | 1 | 173525723 | 1325596 | 4864 | GACCCTGATGRCACTACAGCT | 0.299 | 0.296 | 1.676 | 2.522 | 2.888 |
| 1 | 1 | 173539000 | 16850237 | 4865 | AGATTAGAAGYGACCCTTGTT | 2.965 | 3.014 | 1.463 | 1.778 | 2.700 |
| 1 | 1 | 173548489 | 6681309 | 4866 | AGTCAGAATTKAGTCTGGTAG | 0.471 | 0.464 | 1.283 | 1.400 | 2.492 |
| 1 | 1 | 173558611 | 16850265 | 4867 | CAGGACAAAASGGCATGTAAG | 0.386 | 0.372 | 2.165 | 1.157 | 1.934 |
| 1 | 1 | 173566293 | 4652196 | 4868 | GATCCTCTCAYTCAGTTGTGT | 0.010 | 0.010 | 0.584 | 1.038 | 1.090 |
| 1 | 1 | 173572622 | 16850279 | 4869 | CAATTGCAGGYTCACAGAAAC | 1.855 | 1.775 | 0.467 | 1.104 | 0.992 |
| 1 | 1 | 173579759 | 16850292 | 4870 | GTAGGAAGTCYGTCCTCTGCC | 0.026 | 0.026 | 0.381 | 0.309 | 0.767 |
| 1 | 1 | 173586269 | 4652202 | 4871 | TTCAAGATGASTGGATTCTGG | 0.176 | 0.177 | 0.573 | 0.363 | 0.809 |
| 1 | 1 | 173592666 | — | 4872 | AAGATGTAGAYACACACACAA | 0.149 | 0.149 | 0.039 | 0.347 | 0.767 |
| 1 | 1 | 173599953 | 12131188 | 4873 | TAAATAAATGYCATTTGCTTT | 0.494 | 0.488 | 0.211 | 0.350 | 0.296 |
| 1 | 1 | 173606740 | 11585886 | 4874 | CTGGACATGGRAGGAAACAAA | 0.130 | 0.130 | 0.245 | 0.132 | 0.302 |
| 1 | 1 | 173613449 | 10489303 | 4875 | TAGGTCATACRGATTTCAAAG | 0.751 | 0.746 | 0.238 | 0.170 | 0.318 |
| 1 | 1 | 173624043 | 760690 | 4876 | AATCATTTGCYGAGTTTATAG | 0.294 | 0.292 | 0.327 | 0.220 | 0.348 |
| 1 | 1 | 173632495 | 17379085 | 4877 | AAAACCCACAYGGAAGTCACC | 0.043 | 0.041 | 0.316 | 0.311 | 0.112 |
| 1 | 1 | 173637961 | 17313493 | 4878 | GTTGCAGTAAYTGACTCAGCC | 0.751 | 0.750 | 0.225 | 0.301 | 0.111 |
| 1 | 1 | 173651588 | 4652211 | 4879 | TGTATACATAYCGCCAACAAG | 0.135 | 0.135 | 0.321 | 0.311 | 0.139 |
| 1 | 1 | 173660586 | 2235300 | 4880 | ACCTACTTCTYTGACCAGTTG | 0.459 | 0.448 | 0.468 | 0.173 | 0.176 |
| 1 | 1 | 173667964 | 16850479 | 4881 | TACCTAGAGGYAAGAATTGGC | 0.533 | 0.513 | 0.244 | 0.124 | 0.252 |
| 1 | 1 | 173674919 | — | 4882 | TATATGTGCASACAGGTGCTT | 0.000 | 0.000 | 0.230 | 0.205 | 0.354 |
| 1 | 1 | 173682085 | — | 4883 | TCTCCTTCAGSACCTACAGCT | 0.280 | 0.268 | 0.093 | 0.133 | 0.278 |
| 1 | 1 | 173691633 | 228006 | 4884 | TCAGCCTAAAYGTTTCTTTCT | 0.126 | 0.128 | 0.093 | 0.320 | 0.426 |
| 1 | 1 | 173703286 | 228015 | 4885 | ATAAAACCAGSAGTATGAGCA | 0.001 | 0.002 | 0.132 | 0.389 | 0.405 |
| 1 | 1 | 173710122 | 6661023 | 4886 | GGTCTTTGTCYTTTTAGTGGT | 0.449 | 0.448 | 0.366 | 0.340 | 0.275 |
| 1 | 1 | 173724072 | 13375529 | 4887 | CAGGCAGTCCRCATGGTCTAG | 0.533 | 0.525 | 0.583 | 0.516 | 0.355 |
| 1 | 1 | 173730025 | 10753142 | 4888 | CTTGCCTAGCWCAAACCCAAG | 1.058 | 0.991 | 0.736 | 0.432 | 0.378 |
| 1 | 1 | 173735903 | — | 4889 | TGTTCAAAAGRTTCCTCACTT | 0.685 | 0.705 | 0.999 | 0.425 | 0.278 |
| 1 | 1 | 173741051 | 6672689 | 4890 | GTCTTTAATCRTGTGGAAAGG | 0.320 | 0.319 | 0.727 | 0.601 | 0.483 |
| 1 | 1 | 173748025 | 6667588 | 4891 | GTCATGGGGCRAAACTGAATT | 1.036 | 1.035 | 0.358 | 0.657 | 0.552 |
| 1 | 1 | 173767376 | — | 4892 | CCAAGTGCCCKGTTCCCTCTT | 0.003 | 0.003 | 0.324 | 0.482 | 0.566 |
| 1 | 1 | 173773076 | 6704507 | 4893 | AGTTATTAACYTATTCACACT | 0.148 | 0.148 | 0.427 | 0.615 | 0.597 |
| 1 | 1 | 173784395 | 2068331 | 4894 | GTTTCTAACCYCTGGAATAAA | 0.580 | 0.593 | 0.093 | 0.561 | 0.516 |
| 1 | 1 | 173790737 | 1028304 | 4895 | AACATCAAAAYCATCAGTTGT | 0.570 | 0.583 | 0.599 | 0.524 | 0.466 |
| 2 | 1 | 225466378 | 537250 | 4896 | ACAGATACTCRGTCGGCCCAG | 0.098 | 0.096 | 0.093 | 0.330 | 0.319 |
| 2 | 1 | 225472223 | 484947 | 4897 | ATTGAGCCTGMTCAGTAATGA | 0.249 | 0.256 | 0.058 | 0.139 | 0.319 |
| 2 | 1 | 225479994 | 627512 | 4898 | ATCATACATTYTCTACTTCGT | 0.137 | 0.136 | 0.079 | 0.101 | 0.306 |
| 2 | 1 | 225488350 | 1380024 | 4899 | CTTAATTCTAYTTGAGTCACC | 0.111 | 0.110 | 0.083 | 0.240 | 0.316 |
| 2 | 1 | 225496212 | 682283 | 4900 | GATCCAGGGTRTTATGATGGA | 0.496 | 0.494 | 0.134 | 0.217 | 0.165 |
| 2 | 1 | 225504626 | 12122684 | 4901 | ATTACCCTTTYCTGTGCCCTT | 0.139 | 0.139 | 0.415 | 0.231 | 0.427 |
| 2 | 1 | 225511143 | 1359150 | 4902 | AAGGCCATAGSTAGGAGCATG | 0.484 | 0.464 | 0.505 | 0.208 | 0.490 |
| 2 | 1 | 225519198 | 630685 | 4903 | GGAACCTGCARTGAGAAGTAA | 1.062 | 1.094 | 0.411 | 0.194 | 0.400 |
| 2 | 1 | 225531481 | 12139882 | 4904 | TTGTCACTCAYAGGGCCAATG | 0.330 | 0.344 | 0.431 | 0.906 | 0.494 |
| 2 | 1 | 225537876 | 619422 | 4905 | GGAAAACCCAMAGAAGAATAT | 0.225 | 0.239 | 0.294 | 0.939 | 0.664 |
| 2 | 1 | 225610045 | — | 4906 | AATCCAGCTCRCAAACCAAGA | 0.217 | 0.220 | 0.967 | 0.960 | 0.744 |
| 2 | 1 | 225616273 | 9435811 | 4907 | CCTCTAATGGSTTGGTCACAC | 0.131 | 0.130 | 1.032 | 1.009 | 0.904 |
| 2 | 1 | 225623289 | 12026629 | 4908 | TTCTTTCATGYATCATGTAGA | 2.452 | 2.384 | 0.927 | 0.944 | 0.884 |
| 2 | 1 | 225626459 | — | 4909 | CACGCCTTTCYAGCCTCACAC | 0.596 | 0.592 | 1.169 | 0.927 | 3.052 |
| 2 | 1 | 225633768 | 342782 | 4910 | ATTTATGTATKCAGGTATTCA | 0.030 | 0.030 | 1.729 | 1.041 | 4.062 |
| 2 | 1 | 225645683 | 342785 | 4911 | AGGAGAAGAGMCTTCAAACTG | 0.557 | 0.551 | 0.455 | 1.205 | 3.966 |
| 2 | 1 | 225660552 | — | 4912 | CACATCTACCRACCATCTGAC | 0.927 | 0.883 | 0.414 | 3.775 | 3.710 |
| 2 | 1 | 225667166 | — | 4913 | AGTGGAGGTCRTTAGGAGAGG | 0.319 | 0.312 | 0.603 | 3.761 | 3.447 |
| 2 | 1 | 225673850 | 342818 | 4914 | ACATTTAAATYATCCAGTCTG | 0.513 | 0.500 | 3.447 | 3.735 | 4.665 |
| 2 | 1 | 225680359 | 7526949 | 4915 | CCCATTGTTCRTTTTTGGAAT | 0.441 | 0.436 | 3.789 | 4.364 | 3.966 |
| 2 | 1 | 225687492 | 12041301 | 4916 | AGACAACATCRTGTTCTTCCA | 4.536 | 2.865 | 3.687 | 3.761 | 3.221 |
| 2 | 1 | 225692972 | 16849638 | 4917 | TCATCTCTTAWGCAACTGAAC | 2.478 | 2.365 | 3.966 | 3.108 | 2.686 |
| 2 | 1 | 225703254 | — | 4918 | GCACATTTCAYCAAGCTGATA | 0.397 | 0.398 | 3.397 | 2.886 | 2.545 |
| 2 | 1 | 225722322 | — | 4919 | TGAATATAATYATAAACTGAG | 1.197 | 1.054 | 1.447 | 3.327 | 2.372 |
| 2 | 1 | 225730168 | 9435835 | 4920 | AAGGATCTTASCCTGCATTTT | 0.150 | 0.153 | 0.328 | 2.847 | 2.310 |
| 2 | 1 | 225738651 | 237777 | 4921 | TTACATTACAYTTCTTGACAT | 0.030 | 0.031 | 0.872 | 1.260 | 2.290 |
| 2 | 1 | 225746316 | 9069 | 4922 | TTGGGACACAWTCGTTGGAAC | 0.199 | 0.197 | 0.484 | 0.525 | 2.409 |
| 2 | 1 | 225756176 | 9435838 | 4923 | TCAAAGCCGCWTGAGAAGGTG | 1.719 | 1.730 | 0.550 | 0.591 | 2.457 |
| 2 | 1 | 225765367 | 237799 | 4924 | TTGGGTTCTCYGTTTCAGACT | 0.279 | 0.276 | 0.571 | 0.292 | 0.955 |
| 2 | 1 | 225771581 | 10916465 | 4925 | AGGACCAAGASTAAAGGGAAA | 0.294 | 0.274 | 0.790 | 0.443 | 0.402 |
| 2 | 1 | 225807183 | 16849715 | 4926 | AGGAATTCAAWCCTCAAGACC | 0.139 | 0.136 | 0.117 | 0.487 | 0.391 |
| 2 | 1 | 225815451 | 238098 | 4927 | GAGCCAGGGTYCAGTTTGTGC | 0.652 | 0.637 | 0.245 | 0.485 | 0.315 |
| 2 | 1 | 225829949 | 6587333 | 4928 | GTCATAAATGRGTATTTGTTA | 0.000 | 0.000 | 0.227 | 0.143 | 0.316 |
| 2 | 1 | 225843520 | 12131147 | 4929 | TGTGCCATTCSAATGTGGCTT | 0.708 | 0.712 | 0.255 | 0.153 | 0.418 |
| 2 | 1 | 225845432 | 683116 | 4930 | ATCTGGATAGWGATCACTGAG | 0.242 | 0.241 | 0.194 | 0.283 | 0.377 |
| 2 | 1 | 225862235 | 4925479 | 4931 | AATGTCAACTYGATTTTAGAT | 0.207 | 0.212 | 0.272 | 0.297 | 0.097 |
| 2 | 1 | 225867901 | 482442 | 4932 | GTTACAGACAMACAAGTTAAG | 0.416 | 0.418 | 0.331 | 0.273 | 0.082 |
| 2 | 1 | 225872563 | 500639 | 4933 | AAACGAGTTTRTTACTTTGAG | 0.328 | 0.328 | 0.324 | 0.256 | 0.155 |
| 2 | 1 | 225894215 | — | 4934 | CTTACAGCACWCAAAAGATAA | 0.903 | 0.848 | 0.423 | 0.122 | 0.204 |
| 2 | 1 | 225902995 | 1328219 | 4935 | CTTCTCAGGTRTATGACAGTA | 0.154 | 0.158 | 0.278 | 0.106 | 0.147 |
| 2 | 1 | 225909019 | 7555749 | 4936 | CAACCTTATAMATTATCGGCA | 0.525 | 0.524 | 0.163 | 0.225 | 0.194 |
| 2 | 1 | 225914901 | 4925489 | 4937 | GTGAAGCAATRGCTCTAGATT | 0.047 | 0.045 | 0.026 | 0.235 | 0.138 |
| 2 | 1 | 225921466 | — | 4938 | GGATCTGATGYACAGTAGTCC | 0.046 | 0.038 | 0.194 | 0.211 | 0.172 |
| 2 | 1 | 225930073 | 17355666 | 4939 | GACCGTCAACYGACACAAAAT | 0.099 | 0.099 | 0.167 | 0.133 | 0.219 |
| 2 | 1 | 225938469 | 11805194 | 4940 | CATTTACTGAYGTTTGAACTC | 0.890 | 0.884 | 0.209 | 0.144 | 0.185 |
| 2 | 1 | 225947206 | 12033236 | 4941 | GACTGAATTTYGCATATACAG | 0.387 | 0.389 | 0.325 | 0.129 | 0.209 |
| 2 | 1 | 225949387 | 6671385 | 4942 | TTACATGGTCYTCAACAGCAG | 0.126 | 0.124 | 0.342 | 0.236 | 0.110 |
| 2 | 1 | 225971007 | 927205 | 4943 | AAGAAAGTGAYTCCAGAGACA | 0.484 | 0.438 | 0.212 | 0.264 | 0.213 |
| 2 | 1 | 225982078 | — | 4944 | CCCAGTCTGTYGAATGAAGAA | 0.258 | 0.248 | 0.287 | 0.341 | 0.220 |
| 2 | 1 | 225987741 | 927204 | 4945 | GGGTCCTCTTYCAGAGGTTTG | 0.499 | 0.516 | 0.249 | 0.155 | 0.262 |
| 3 | 1 | 227029796 | 12135204 | 4946 | ATCCATGATTRCAAGTGATTG | 0.273 | 0.270 | 0.441 | 0.265 | 0.471 |
| 3 | 1 | 227038868 | 200580 | 4947 | TTGGTTGTAARATCTTAGAAA | 0.155 | 0.159 | 0.101 | 0.371 | 0.406 |
| 3 | 1 | 227056681 | 853465 | 4948 | CTAGAAATCCWGCAGACAGTA | 0.351 | 0.352 | 0.187 | 0.466 | 0.267 |
| 3 | 1 | 227062143 | 699900 | 4949 | CTATTTACCASAAGCAAGCAT | 0.232 | 0.237 | 0.255 | 0.257 | 0.298 |
| 3 | 1 | 227068583 | 10779849 | 4950 | TGCAGATGGTYTCACTCACTC | 0.524 | 0.532 | 0.399 | 0.230 | 0.309 |
| 3 | 1 | 227082261 | 3761950 | 4951 | GCTAAGCCAGRCAGTAACCCT | 0.464 | 0.476 | 0.495 | 0.312 | 0.696 |
| 3 | 1 | 227102376 | 2225146 | 4952 | GGTTGTTTTCRTTAGCAGCTG | 0.663 | 0.656 | 0.420 | 0.334 | 0.414 |
| 3 | 1 | 227108750 | 16852170 | 4953 | CCTTCACTAAYGAGGCTAGAC | 0.612 | 0.597 | 0.467 | 0.265 | 0.389 |
| 3 | 1 | 227115479 | 2148964 | 4954 | GGCCTTTGCAKTACCCTCTCT | 0.078 | 0.068 | 0.393 | 0.911 | 0.568 |
| 3 | 1 | 227122216 | 2493144 | 4955 | AAGAGTCATTKTTGCAGATTC | 0.644 | 0.621 | 0.176 | 0.774 | 0.571 |
| 3 | 1 | 227129065 | 2296800 | 4956 | AAATGTATTAYGCTATGTTAT | 0.268 | 0.301 | 0.868 | 0.599 | 0.475 |
| 3 | 1 | 227135608 | 3789662 | 4957 | AAATCCTCACRTTTTACAACA | 0.141 | 0.139 | 0.889 | 0.751 | 0.479 |
| 3 | 1 | 227147004 | 11122575 | 4958 | AAAATCCTTCRTGTCATTTGC | 1.937 | 1.984 | 0.587 | 0.589 | 0.485 |
| 3 | 1 | 227153414 | 11122576 | 4959 | TTTCATTGTCYCAATATTCCC | 0.263 | 0.254 | 0.900 | 0.541 | 0.402 |
| 3 | 1 | 227160094 | 9804147 | 4960 | CTTGGAAGAGRCATGTGAGCC | 0.067 | 0.067 | 0.887 | 0.426 | 0.456 |
| 3 | 1 | 227167331 | 7548604 | 4961 | ACAGAATATTSCTGGCAAAAT | 1.051 | 0.999 | 0.169 | 0.490 | 0.410 |
| 3 | 1 | 227174605 | 2478518 | 4962 | TTACTTATGTSCTGGTTTATG | 0.179 | 0.178 | 0.153 | 0.483 | 0.449 |
| 3 | 1 | 227186246 | 2478542 | 4963 | GTGAAGCTTCYGAGCATTTGT | 0.006 | 0.006 | 0.285 | 0.223 | 0.525 |
| 3 | 1 | 227196981 | 2493148 | 4964 | TTTACCTAAGYGCATCTCTGT | 0.252 | 0.262 | 0.048 | 0.250 | 0.540 |
| 3 | 1 | 227205229 | 2282319 | 4965 | CCGACCACAAYGAGTTCTGGA | 0.527 | 0.528 | 0.256 | 0.293 | 0.903 |
| 3 | 1 | 227213689 | 6677009 | 4966 | TCTTGGACTCYGAAGAGTTGA | 0.088 | 0.095 | 0.402 | 0.287 | 0.663 |
| 3 | 1 | 227219919 | 11122597 | 4967 | ATTCTTCACCRGCTCTAGGAT | 0.911 | 0.895 | 0.397 | 0.349 | 0.818 |
| 3 | 1 | 227231640 | 12082061 | 4968 | GCATAACACAYGGCTAGCGCA | 0.439 | 0.414 | 0.581 | 0.819 | 1.742 |
| 3 | 1 | 227238473 | 11122604 | 4969 | TTTCTAATCTRTGTTCCATAG | 0.258 | 0.265 | 0.678 | 1.044 | 2.642 |
| 3 | 1 | 227246905 | 10864783 | 4970 | TAGTATACACRAGCTATCTAA | 0.969 | 0.975 | 1.022 | 1.241 | 2.983 |
| 3 | 1 | 227258633 | 1202525 | 4971 | TGAGGCTGCASGGAGGCCAGA | 0.414 | 0.388 | 1.250 | 2.377 | 3.250 |
| 3 | 1 | 227265670 | 1202534 | 4972 | GTTTTCCCCCRAAAAGGTTGG | 1.595 | 1.513 | 1.512 | 3.676 | 3.127 |
| 3 | 1 | 227270465 | — | 4973 | ATGGCCCCGTRAGGTTAGCGG | 1.029 | 1.010 | 2.517 | 3.665 | 3.447 |
| 3 | 1 | 227281064 | 6673201 | 4974 | AGGTGTCGCARAGATTAAGGC | 0.939 | 0.918 | 4.364 | 3.886 | 3.585 |
| 3 | 1 | 227291324 | 16852841 | 4975 | TGCTCAGCTCYAAAAACTCCA | 3.457 | 3.237 | 4.966 | 3.735 | 3.623 |
| 3 | 1 | 227301328 | 1999903 | 4976 | CTCTGAGGAAYGAAGACTTAG | 4.565 | 4.153 | 4.966 | 4.011 | 3.534 |
| 3 | 1 | 227307747 | 16852927 | 4977 | ACCGGCTCCTYTCCTTTTGCT | 1.713 | 1.578 | 4.364 | 4.011 | 3.386 |
| 3 | 1 | 227320088 | 1202594 | 4978 | TTTTTCCAATKGTAGAGAGAG | 0.031 | 0.031 | 3.159 | 3.604 | 3.312 |
| 3 | 1 | 227328505 | 4028814 | 4979 | AATTCTCTACYTCAGAAAGCA | 0.078 | 0.076 | 0.771 | 2.759 | 2.944 |
| 3 | 1 | 227334767 | 6704527 | 4980 | TTACTTCCTGKTAAGAGGTCT | 0.800 | 0.818 | 0.138 | 1.946 | 2.526 |
| 3 | 1 | 227343240 | 3762404 | 4981 | AGACGGAAGTYAGAGATTTTT | 0.581 | 0.550 | 0.244 | 0.662 | 2.282 |
| 3 | 1 | 227354652 | 4846878 | 4982 | AAGAAGGCCAYAGACCAGGAA | 0.053 | 0.054 | 0.544 | 0.238 | 2.167 |
| 3 | 1 | 227362159 | 11122622 | 4983 | GGGCAGTCTAYTTAGAAATTG | 0.344 | 0.338 | 0.447 | 0.294 | 1.379 |
| 3 | 1 | 227372902 | 7533671 | 4984 | CAGTCATGGTRTAAAGGGCTC | 0.797 | 0.812 | 0.288 | 0.314 | 0.347 |
| 3 | 1 | 227381046 | 6667425 | 4985 | GTCAGGCAGTYCTCAAGCAAT | 0.624 | 0.586 | 0.348 | 0.242 | 0.093 |
| 3 | 1 | 227388039 | 6673085 | 4986 | CAGCTCTACTKCTTTATGCTA | 0.091 | 0.092 | 0.272 | 0.127 | 0.102 |
| 3 | 1 | 227394719 | 6679473 | 4987 | TGGATGCCTCYAAATTATTCT | 0.228 | 0.238 | 0.161 | 0.187 | 0.108 |
| 3 | 1 | 227396393 | 16853489 | 4988 | ATTAAGTTAAWCATGAAGATC | 0.146 | 0.147 | 0.048 | 0.129 | 0.046 |
| 3 | 1 | 227417148 | 7413309 | 4989 | TGTTGTTAAAYGGTATGTAAC | 0.548 | 0.544 | 0.124 | 0.033 | 0.031 |
| 3 | 1 | 227432026 | 6541249 | 4990 | ATTTCAGACTYTTAAGAATTA | 0.039 | 0.040 | 0.074 | 0.012 | 0.075 |
| 3 | 1 | 227458465 | 4551581 | 4991 | TAAATTACACMATAAATCATT | 0.442 | 0.455 | 0.055 | 0.013 | 0.071 |
| 3 | 1 | 227469378 | 4506448 | 4992 | TGCTCAAAAAYCATGGAAGAA | 0.008 | 0.008 | 0.014 | 0.020 | 0.024 |
| 3 | 1 | 227486664 | 4332346 | 4993 | ATTACTTTGARAGCAACTGTG | 0.122 | 0.119 | 0.022 | 0.063 | 0.052 |
| 3 | 1 | 227496553 | 6679453 | 4994 | ATTAATTTACRCAAAGTCATC | 0.151 | 0.152 | 0.017 | 0.042 | 0.138 |
| 3 | 1 | 227511349 | 6663912 | 4995 | CAAACTACCCMGAGTTAGCAC | 0.103 | 0.104 | 0.133 | 0.051 | 0.257 |
| 3 | 1 | 227518492 | 6541254 | 4996 | TTGGCATTCARTGATCTGAAA | 0.375 | 0.388 | 0.190 | 0.139 | 0.234 |
| 3 | 1 | 227541095 | 7518479 | 4997 | GTTGCCTGTCRATGGCAGTCT | 0.682 | 0.685 | 0.169 | 0.338 | 0.157 |
| 3 | 1 | 227551201 | 9431925 | 4998 | ACTTCCTTCCMTTCCGTTAGA | 0.309 | 0.307 | 0.543 | 0.580 | 0.191 |
| 3 | 1 | 227560738 | 10864669 | 4999 | TCTGCAAACARATCAAAACAG | 0.104 | 0.106 | 0.746 | 0.539 | 0.134 |
| 4 | 1 | 236154391 | 16838637 | 5000 | TCGGTCTCTGRAAAAAAATGG | 0.649 | 0.661 | 0.578 | 0.423 | 0.609 |
| 4 | 1 | 236161654 | 7541783 | 5001 | CTTCCTTTTGWCAGCAGGAGA | 0.994 | 0.971 | 0.677 | 0.453 | 0.576 |
| 4 | 1 | 236168996 | 10925949 | 5002 | AGATTAATGTMCCTGATTTAA | 0.471 | 0.482 | 0.601 | 0.467 | 0.443 |
| 4 | 1 | 236176629 | 6688537 | 5003 | ATGGCAGCCCMTGCCACTTTT | 0.331 | 0.331 | 0.663 | 0.529 | 0.478 |
| 4 | 1 | 236183565 | 2355237 | 5004 | AAAGAATTAGRTGATCGATAC | 0.280 | 0.277 | 0.385 | 0.574 | 0.444 |
| 4 | 1 | 236196816 | 10802795 | 5005 | CTCAATGTTCYGAATTATGAG | 0.844 | 0.802 | 0.348 | 0.545 | 0.484 |
| 4 | 1 | 236203307 | 6429154 | 5006 | TTAATACGGCRGATTTCTTTC | 0.176 | 0.170 | 0.456 | 0.455 | 0.490 |
| 4 | 1 | 236209843 | 10925956 | 5007 | GCCACTCAACSCCAGACTTTT | 0.321 | 0.315 | 0.516 | 0.444 | 0.453 |
| 4 | 1 | 236217552 | 6657343 | 5008 | TAACAAACACWTACTTCACAA | 0.690 | 0.661 | 0.416 | 0.454 | 0.390 |
| 4 | 1 | 236227047 | 11579382 | 5009 | TGTTTGTAATSTTATGCTGCT | 0.548 | 0.522 | 0.514 | 0.401 | 0.617 |
| 4 | 1 | 236235692 | 11585281 | 5010 | ATTTTTAAAAYTCTGTGATAC | 0.000 | 0.000 | 0.534 | 0.220 | 0.620 |
| 4 | 1 | 236237040 | 934344 | 5011 | GTTTTCAAACYATTTTATAAG | 0.000 | 0.000 | 0.252 | 0.226 | 1.292 |
| 4 | 1 | 236245819 | 10925972 | 5012 | TACCAATTTAKTTCAGGGAGA | 0.394 | 0.387 | 0.089 | 0.918 | 1.426 |
| 4 | 1 | 236257599 | 865213 | 5013 | ATCTTAGGATYAGCGTTCACT | 0.115 | 0.113 | 0.091 | 0.873 | 1.613 |
| 4 | 1 | 236274676 | 658842 | 5014 | AAAACATTATWAAACCCGGTT | 0.158 | 0.158 | 0.889 | 2.569 | 1.847 |
| 4 | 1 | 236286446 | 6661317 | 5015 | CAGAAACACARAATCAAATAC | 0.343 | 0.351 | 1.017 | 2.365 | 1.952 |
| 4 | 1 | 236300211 | 1110615 | 5016 | TCTGAAAATCRCCATCTGTAA | 2.208 | 2.317 | 3.735 | 2.650 | 1.742 |
| 4 | 1 | 236311513 | 6429161 | 5017 | TCTGCATAGAWATCTAATTCA | 0.000 | 0.000 | 3.687 | 2.691 | 1.509 |
| 4 | 1 | 236324113 | 477507 | 5018 | CACCAACTAGRTTGAAAATTG | 3.741 | 3.312 | 3.924 | 2.327 | 1.509 |
| 4 | 1 | 236339319 | 613228 | 5019 | TCCTTTTAATWTAAATATGTT | 0.297 | 0.379 | 2.589 | 2.459 | 1.749 |
| 4 | 1 | 236365881 | 17598757 | 5020 | ACTCTCTCTAYGTCTCTATAT | 0.981 | 0.944 | 1.965 | 2.235 | 2.389 |
| 4 | 1 | 236372267 | — | 5021 | TCCTGGAGACWCATCAGCCCA | 0.062 | 0.062 | 0.145 | 1.326 | 2.635 |
| 4 | 1 | 236381514 | 10495448 | 5022 | TTTTAATCCCRTAGAGCCAGA | 0.043 | 0.043 | 0.079 | 1.654 | 2.886 |
| 4 | 1 | 236391685 | 10926012 | 5023 | TCGTCTCAAARTAGTCAAGTA | 0.135 | 0.132 | 0.002 | 0.807 | 3.173 |
| 4 | 1 | 236403350 | 4659554 | 5024 | AGGGGCTCACRTCTCAGTTGC | 0.021 | 0.021 | 0.246 | 0.898 | 2.701 |
| 4 | 1 | 236415559 | — | 5025 | TAATAGCACCKGAAATAAAGA | 0.000 | 0.000 | 1.231 | 0.930 | 2.549 |
| 4 | 1 | 236429275 | 12404902 | 5026 | TCACCTCTTGYCAGTTAATAA | 1.195 | 1.253 | 1.706 | 1.466 | 0.868 |
| 4 | 1 | 236436879 | 6671685 | 5027 | TCCAGCAAAAYCGTTTATTTT | 2.138 | 1.968 | 2.197 | 1.658 | 0.771 |
| 4 | 1 | 236443827 | 10926032 | 5028 | GATCTCACGTWGACTCTGTCT | 0.000 | 0.000 | 2.299 | 1.874 | 0.555 |
| 4 | 1 | 236450356 | 4659556 | 5029 | ATACCCATTAYGTAGCTTTTC | 0.935 | 0.986 | 1.995 | 1.793 | 0.541 |
| 4 | 1 | 236456129 | 10926042 | 5030 | TTAAAACATGYCTCATGTGAA | 1.160 | 1.149 | 0.932 | 1.389 | 0.530 |
| 4 | 1 | 236461974 | 4659557 | 5031 | AGGGCGTGCARCTGAGCGGTG | 0.000 | 0.000 | 0.629 | 0.862 | 0.557 |
| 4 | 1 | 236467860 | 12744283 | 5032 | TAGAACTTCCRTCCCTCATAA | 0.115 | 0.113 | 0.243 | 0.231 | 0.679 |
| 4 | 1 | 236474186 | 12407556 | 5033 | ACATGCATAGRTGTTGTTCTG | 0.046 | 0.046 | 0.012 | 0.166 | 0.659 |
| 4 | 1 | 236479700 | 2169453 | 5034 | AAGAAAAAATYTACACTTGGA | 0.051 | 0.053 | 0.003 | 0.062 | 0.458 |
| 4 | 1 | 236485357 | 12743555 | 5035 | TAGGTAGAAAKAGGATTACAG | 0.139 | 0.143 | 0.003 | 0.012 | 0.133 |
| 4 | 1 | 236493915 | 7512443 | 5036 | AGGGAGTATTWATTGAGCAGT | 0.050 | 0.042 | 0.009 | 0.018 | 0.118 |
| 4 | 1 | 236500948 | 12073505 | 5037 | TTGATGGGAGMCTTAGGACAT | 0.104 | 0.084 | 0.056 | 0.032 | 0.068 |
| 4 | 1 | 236508885 | 2883722 | 5038 | TGAGGTTAAGRTTTAGAATTT | 0.225 | 0.226 | 0.103 | 0.039 | 0.013 |
| 4 | 1 | 236518457 | 10926084 | 5039 | TTTTCTACAAYGAATGATTCT | 0.550 | 0.529 | 0.164 | 0.053 | 0.011 |
| 4 | 1 | 236528295 | 12075485 | 5040 | GTGAAGTCCCRCAAGAGAGGT | 0.416 | 0.415 | 0.188 | 0.083 | 0.017 |
| 4 | 1 | 236533851 | 12239766 | 5041 | GAAAAAGTGCRCAAAATCTGC | 0.334 | 0.323 | 0.202 | 0.084 | 0.019 |
| 4 | 1 | 236545865 | — | 5042 | CATCACGTTGMATTTTTAAAT | 0.174 | 0.165 | 0.174 | 0.095 | 0.033 |
| 4 | 1 | 236557741 | — | 5043 | TAGAGATCCCYATAGTCTATC | 0.261 | 0.284 | 0.100 | 0.112 | 0.028 |
| 4 | 1 | 236561988 | — | 5044 | AGCACCTTTTYCAGTAGTCTC | 0.443 | 0.461 | 0.079 | 0.066 | 0.035 |
| 4 | 1 | 236584566 | 12143985 | 5045 | GTGACATTCAYGTATATAGTT | 0.113 | 0.116 | 0.116 | 0.065 | 0.164 |
| 4 | 1 | 236591444 | 16839420 | 5046 | TTTTCCTACTRTTGAATATGC | 0.262 | 0.231 | 0.092 | 0.043 | 0.199 |
| 4 | 1 | 236597432 | 1545727 | 5047 | TCAGTGAATTWTGATTCCTCC | 0.302 | 0.281 | 0.086 | 0.055 | 0.212 |
| 4 | 1 | 236605709 | 16839444 | 5048 | GCGAGATTATRGGAGAGAATA | 0.168 | 0.169 | 0.075 | 0.276 | 0.272 |
| 4 | 1 | 236625805 | — | 5049 | TTTTAAGACAMTGACATACTT | 0.431 | 0.423 | 0.094 | 0.278 | 0.240 |
| 4 | 1 | 236632495 | 12071494 | 5050 | TTGAGCTAACYGGCGCAACAT | 0.114 | 0.117 | 0.539 | 0.418 | 0.360 |
| 4 | 1 | 236638476 | 10737842 | 5051 | AACATTGAACYTGCAGAATGT | 0.331 | 0.341 | 0.684 | 0.590 | 0.406 |
| 4 | 1 | 236644953 | 6666380 | 5052 | TGAGAGCCCARAGTGATTGAG | 1.572 | 1.626 | 0.782 | 0.525 | 0.425 |
| 5 | 2 | 20891681 | 661929 | 5053 | AGGAAACTCTRTGTTATTATG | 0.058 | 0.057 | 0.275 | 0.303 | 0.182 |
| 5 | 2 | 20901381 | 16988063 | 5054 | CTACTTCATCRATCCATAAGG | 0.054 | 0.054 | 0.090 | 0.224 | 0.184 |
| 5 | 2 | 20910027 | 535022 | 5055 | CTCATACCACYAGAGAGACGT | 0.347 | 0.326 | 0.098 | 0.197 | 0.168 |
| 5 | 2 | 20916346 | 16982026 | 5056 | TACTTCACCAYTTAAACCTCC | 0.411 | 0.410 | 0.138 | 0.090 | 0.277 |
| 5 | 2 | 20926604 | 649018 | 5057 | TGAAAATCAARCTCAGTCTAT | 0.359 | 0.370 | 0.213 | 0.077 | 0.283 |
| 5 | 2 | 20933936 | 16988079 | 5058 | TCATGACTTCYCTGCTGCCAT | 0.197 | 0.191 | 0.246 | 0.127 | 0.243 |
| 5 | 2 | 20952607 | 340602 | 5059 | TGACTGCCTGYTTCATTTGGT | 0.217 | 0.221 | 0.162 | 0.273 | 0.104 |
| 5 | 2 | 20968923 | 6709100 | 5060 | GCCCTCCATAYGGTTCAGAGA | 0.480 | 0.495 | 0.167 | 0.342 | 0.108 |
| 5 | 2 | 20975686 | 6743521 | 5061 | AAAGCCAATGRGCCTAAAATT | 0.172 | 0.171 | 0.361 | 0.277 | 0.153 |
| 5 | 2 | 20984059 | 11891188 | 5062 | AAAGTCAGAGWTCTTCGGAGG | 0.478 | 0.485 | 0.540 | 0.202 | 0.156 |
| 5 | 2 | 20992564 | 1437404 | 5063 | AATATAAAAGYTAAAAGGCAA | 0.824 | 0.828 | 0.413 | 0.237 | 0.164 |
| 5 | 2 | 20998690 | 16988112 | 5064 | TCCTCCTCCARCTCTTACGTT | 0.603 | 0.604 | 0.376 | 0.305 | 0.166 |
| 5 | 2 | 21005714 | 28538171 | 5065 | AAGACACCCAYGTCCTGAGAG | 0.178 | 0.182 | 0.316 | 0.201 | 0.115 |
| 5 | 2 | 21023010 | 6722139 | 5066 | AGCACACATGMGGAATTTTAT | 0.082 | 0.082 | 0.204 | 0.245 | 0.174 |
| 5 | 2 | 21047370 | 4468779 | 5067 | AAGAACCCATYCTAGCAGGTC | 0.379 | 0.372 | 0.068 | 0.230 | 0.179 |
| 5 | 2 | 21060496 | 4606879 | 5068 | GTCCTAGGAARATGAAAAAGA | 0.473 | 0.479 | 0.112 | 0.088 | 0.240 |
| 5 | 2 | 21100722 | 17397826 | 5069 | AGAATGATTGYGTTTTTGATT | 0.043 | 0.055 | 0.227 | 0.107 | 0.259 |
| 5 | 2 | 21107056 | 17041679 | 5070 | TAGAACAAATYTGTGCTGTTG | 0.385 | 0.386 | 0.129 | 0.121 | 0.309 |
| 5 | 2 | 21112994 | 17041694 | 5071 | AGCTGCACAAYCCACCTTGCA | 0.535 | 0.487 | 0.192 | 0.292 | 0.278 |
| 5 | 2 | 21124837 | 4665642 | 5072 | CCAGTGGAGARTTTCCATCTC | 0.021 | 0.022 | 0.259 | 0.270 | 0.250 |
| 5 | 2 | 21137405 | 1042031 | 5073 | ATATGGAATTYTTGAGTAACT | 0.620 | 0.626 | 0.411 | 0.335 | 1.064 |
| 5 | 2 | 21149196 | 673548 | 5074 | CAAAAATACCRATTTGACAAG | 0.161 | 0.167 | 0.341 | 0.461 | 1.039 |
| 5 | 2 | 21157019 | 3791981 | 5075 | TGATCTCTCCRGAGCTATTGT | 0.925 | 0.917 | 0.596 | 0.467 | 0.978 |
| 5 | 2 | 21162692 | 17240674 | 5076 | CAAAATGTCTKGATTTCATTG | 0.285 | 0.281 | 0.609 | 1.536 | 0.919 |
| 5 | 2 | 21172253 | 17240549 | 5077 | ACTCCTCAATRACTGTTTTAA | 0.802 | 0.757 | 0.703 | 1.628 | 0.947 |
| 5 | 2 | 21182206 | 585967 | 5078 | TTCTCTGGGAMCAGCCCAGTT | 0.621 | 0.618 | 2.097 | 1.396 | 0.952 |
| 5 | 2 | 21188053 | 11892073 | 5079 | GCATAAACATRCGGAAATACC | 0.404 | 0.403 | 2.202 | 1.345 | 0.915 |
| 5 | 2 | 21199203 | — | 5080 | GCCGTCCATCYATCCGTTACT | 3.828 | 3.687 | 1.756 | 1.193 | 1.102 |
| 5 | 2 | 21204658 | 594677 | 5081 | ACTGAGTAATYGTCTAATGAA | 0.075 | 0.075 | 1.382 | 1.173 | 1.040 |
| 5 | 2 | 21217337 | 17399144 | 5082 | ATGCTTCAATRAACAGCCAGT | 0.198 | 0.196 | 1.386 | 1.035 | 0.992 |
| 5 | 2 | 21223437 | 675430 | 5083 | CATTTTAATGMGGACAAATCT | 0.289 | 0.286 | 0.130 | 1.080 | 1.056 |
| 5 | 2 | 21229137 | 645456 | 5084 | CCTTCATCATYGATCTCTGGA | 0.358 | 0.334 | 0.234 | 1.106 | 1.138 |
| 5 | 2 | 21233518 | 312942 | 5085 | GTATTCCTCCYTACCTGATTT | 0.349 | 0.350 | 0.451 | 0.202 | 1.344 |
| 5 | 2 | 21253964 | 312957 | 5086 | ATGATTGATARTTAATTTACT | 0.484 | 0.451 | 0.479 | 0.437 | 1.386 |
| 5 | 2 | 21259931 | — | 5087 | AGGGTTGTGTKAGGAAGAGAT | 0.898 | 0.878 | 0.355 | 0.520 | 1.409 |
| 5 | 2 | 21270933 | 1517471 | 5088 | TGTATAGTTCMTCCTAATATC | 0.398 | 0.392 | 0.690 | 0.932 | 0.595 |
| 5 | 2 | 21282720 | 11687710 | 5089 | CAGTTCCTACYTTGTTGAGTT | 0.038 | 0.037 | 0.750 | 1.161 | 0.724 |
| 5 | 2 | 21294098 | 17041836 | 5090 | CATTAAGTCCRATCTGTCCTT | 1.225 | 1.217 | 1.262 | 1.127 | 0.740 |
| 5 | 2 | 21308834 | 506585 | 5091 | CTCTACTAGGRCTACAAGCTT | 0.519 | 0.527 | 1.639 | 1.118 | 0.704 |
| 5 | 2 | 21312943 | 2030195 | 5092 | CAATTTAATARAATCATTTTC | 1.700 | 1.753 | 1.595 | 0.980 | 0.650 |
| 5 | 2 | 21334933 | 4387830 | 5093 | TATACAACTTRAGAAACAAAG | 1.032 | 0.994 | 1.041 | 0.963 | 0.578 |
| 5 | 2 | 21339837 | 312052 | 5094 | TGCCTTCTATRGAATATGTCT | 0.113 | 0.111 | 1.080 | 0.964 | 0.536 |
| 5 | 2 | 21350446 | 437775 | 5095 | CCCATTTCAGRTTGCCTTTCT | 0.179 | 0.178 | 0.341 | 0.555 | 0.683 |
| 5 | 2 | 21360686 | 13420469 | 5096 | TCTCACTCTGYTCAGATTGCT | 0.568 | 0.567 | 0.110 | 0.421 | 0.829 |
| 5 | 2 | 21371828 | 10166647 | 5097 | ATACATTTTGYCTGTGTTCTA | 0.274 | 0.274 | 0.089 | 0.101 | 0.998 |
| 5 | 2 | 21386917 | 4665243 | 5098 | GCAAACAGCTRTCGCAAGCCA | 0.189 | 0.188 | 0.073 | 0.167 | 0.767 |
| 5 | 2 | 21395207 | 1397400 | 5099 | GATTACAGAAKTCTCCTTTGT | 0.034 | 0.035 | 0.023 | 0.355 | 0.666 |
| 5 | 2 | 21403500 | 17042055 | 5100 | TATGCATGCAKCCTGCACCTC | 0.094 | 0.090 | 0.293 | 0.515 | 0.404 |
| 5 | 2 | 21412996 | 4665662 | 5101 | TGCAAATGAAKCTATTCCTGA | 0.211 | 0.208 | 0.590 | 0.459 | 0.229 |
| 5 | 2 | 21414379 | 13396400 | 5102 | GAAGGTGGAARTGGAATAAAA | 1.514 | 1.392 | 0.856 | 0.411 | 0.400 |
| 6 | 2 | 49817753 | 1022369 | 5103 | ATATAGCTCAMCAAAAATATT | 0.450 | 0.424 | 1.326 | 0.817 | 0.697 |
| 6 | 2 | 49820321 | 17180320 | 5104 | TTAATAATTTSAGCACTGTCA | 0.403 | 0.396 | 1.215 | 0.886 | 0.654 |
| 6 | 2 | 49826561 | 17180439 | 5105 | ATTGAATTGASTCTTCCAGGG | 1.506 | 1.592 | 0.680 | 1.065 | 0.663 |
| 6 | 2 | 49856216 | 10185615 | 5106 | TATTAATCTTMTTTGGACACG | 0.007 | 0.007 | 0.629 | 1.205 | 0.675 |
| 6 | 2 | 49869232 | 13407557 | 5107 | ATGCAATGCARTAAAACACTG | 0.648 | 0.618 | 0.850 | 0.683 | 0.683 |
| 6 | 2 | 49876262 | 12713075 | 5108 | TGGTTACTATYCTCTCCCCAG | 0.300 | 0.277 | 0.435 | 0.583 | 0.786 |
| 6 | 2 | 49894129 | 6737293 | 5109 | TGTTCCTTCTYTTTGGGCATA | 0.901 | 0.906 | 0.599 | 0.615 | 1.409 |
| 6 | 2 | 49908869 | 870168 | 5110 | AGCACTTCCCRTTTATGAGAG | 0.555 | 0.545 | 0.391 | 0.268 | 0.929 |
| 6 | 2 | 49921274 | 10174758 | 5111 | CTACTGTGACWATATATTTAA | 0.409 | 0.406 | 0.483 | 0.473 | 1.258 |
| 6 | 2 | 49933375 | 2350705 | 5112 | TTCCCAAAGGSTAACGGTAAC | 0.074 | 0.071 | 0.225 | 0.948 | 1.170 |
| 6 | 2 | 49948039 | 7584378 | 5113 | CTCTATGTCAMTGTCCGTCTA | 0.492 | 0.494 | 0.291 | 0.869 | 1.478 |
| 6 | 2 | 49958503 | 17795388 | 5114 | GATTTTTTAARTCAAAAGAAA | 0.176 | 0.175 | 1.095 | 1.153 | 2.016 |
| 6 | 2 | 49969040 | 17039353 | 5115 | GTTTCCCTGAYAGCCATGGCT | 0.805 | 0.803 | 1.106 | 1.064 | 2.682 |
| 6 | 2 | 49991254 | 10495984 | 5116 | AATGATTTATSTGTTCCTATG | 2.244 | 2.279 | 1.707 | 2.081 | 2.920 |
| 6 | 2 | 49999544 | 17039373 | 5117 | TTTTCTACCAYACTTTTTTCA | 0.041 | 0.040 | 1.682 | 2.662 | 2.829 |
| 6 | 2 | 50004404 | 17490406 | 5118 | GTTTATGTGAYTGTGACATTC | 1.689 | 1.693 | 2.435 | 3.187 | 2.569 |
| 6 | 2 | 50010410 | 1981797 | 5119 | AATTTTTCTCYTTAATATATC | 0.213 | 0.212 | 2.258 | 3.258 | 2.432 |
| 6 | 2 | 50037491 | 1156742 | 5120 | AATGGTATAARAATATTAGGG | 2.618 | 2.545 | 3.275 | 2.886 | 2.518 |
| 6 | 2 | 50048374 | 17039425 | 5121 | CTATTTGCCTRTGATTTTTAA | 1.846 | 1.799 | 3.303 | 2.512 | 2.671 |
| 6 | 2 | 50055972 | 12465974 | 5122 | ATCAAAGATTYCAATTCTTGG | 2.962 | 3.009 | 3.364 | 2.444 | 2.665 |
| 6 | 2 | 50064931 | 10495987 | 5123 | TTGGAAGCTAMTGATTATTCT | 1.835 | 1.901 | 2.362 | 2.249 | 2.575 |
| 6 | 2 | 50077180 | 6758043 | 5124 | ATCATTGCTTRTGTAATCTGA | 0.548 | 0.539 | 1.855 | 2.738 | 2.420 |
| 6 | 2 | 50083309 | 12618646 | 5125 | TAGTTGCTTCRTTGCGCTTAC | 0.738 | 0.723 | 1.215 | 2.305 | 2.425 |
| 6 | 2 | 50089802 | 1363046 | 5126 | CTGTTTAAAAWGTATGCAATG | 0.429 | 0.449 | 1.267 | 2.113 | 2.252 |
| 6 | 2 | 50095750 | 6713560 | 5127 | AGCTTTTAACRGTTCTCAAAG | 1.039 | 1.073 | 1.404 | 1.467 | 2.497 |
| 6 | 2 | 50101824 | 17039579 | 5128 | ACAGGAAATAWCACGGATTCA | 1.856 | 1.760 | 1.294 | 1.021 | 2.086 |
| 7 | 2 | 50107806 | 17039592 | 5129 | GGGTGCTTAGWAATTACTTAT | 0.663 | 0.680 | 1.326 | 1.111 | 1.801 |
| 7 | 2 | 50114259 | 10495989 | 5130 | CCAAAGCTCARTTTGTCTTGG | 0.235 | 0.203 | 0.930 | 0.914 | 1.050 |
| 7 | 2 | 50127794 | 17439837 | 5131 | CCCAGCACAAYGTGGCAGCCA | 0.316 | 0.334 | 0.321 | 0.854 | 0.613 |
| 7 | 2 | 50135085 | 1897924 | 5132 | TGTAGAGAAGRTAGAAGAACA | 0.251 | 0.259 | 0.181 | 0.605 | 0.628 |
| 7 | 2 | 50142762 | 1421577 | 5133 | AGAGATACAAMATAATAATTC | 0.557 | 0.563 | 0.216 | 0.165 | 0.463 |
| 7 | 2 | 50148980 | 10495992 | 5134 | ACACCAAATAKTATTTCATGA | 0.245 | 0.245 | 0.220 | 0.074 | 0.498 |
| 7 | 2 | 50155053 | 6731061 | 5135 | TTCAACATTAYTCACAGAAGG | 0.266 | 0.276 | 0.180 | 0.144 | 0.321 |
| 7 | 2 | 50161986 | 4971634 | 5136 | TCTTCCTGGARAGAAAGTCCT | 0.322 | 0.327 | 0.074 | 0.097 | 0.109 |
| 7 | 2 | 50168831 | 17039678 | 5137 | TGGGTAACTAYGTGTCAAGTG | 0.144 | 0.148 | 0.166 | 0.136 | 0.098 |
| 7 | 2 | 50174623 | 2117560 | 5138 | AAAATACATTYATGTACCTTA | 0.076 | 0.073 | 0.099 | 0.075 | 0.095 |
| 7 | 2 | 50180362 | 10191989 | 5139 | CTCTTTGAAAYGGGCAAGCTA | 0.637 | 0.609 | 0.135 | 0.090 | 0.093 |
| 7 | 2 | 50187712 | 12470518 | 5140 | AGTCAAAAACYAGAATCTCTC | 0.002 | 0.002 | 0.121 | 0.152 | 0.090 |
| 7 | 2 | 50194688 | 17039734 | 5141 | ATAGATCTAAMCTAAGTGAGA | 0.588 | 0.577 | 0.213 | 0.114 | 0.058 |
| 7 | 2 | 50201562 | 17039741 | 5142 | TTCATTTTAGYTCTTCACTCT | 0.172 | 0.177 | 0.211 | 0.132 | 0.100 |
| 7 | 2 | 50208052 | 1563021 | 5143 | CAAAGAAGGGYCACAGAAACC | 0.383 | 0.378 | 0.270 | 0.165 | 0.099 |
| 7 | 2 | 50215591 | 10490244 | 5144 | AACACTATTAYGAGAATGCTT | 0.603 | 0.586 | 0.200 | 0.091 | 0.147 |
| 7 | 2 | 50225689 | 1563026 | 5145 | TGTTCATTAGSTTTTACAGTG | 0.106 | 0.106 | 0.224 | 0.225 | 0.137 |
| 7 | 2 | 50233983 | 17039807 | 5146 | CTGATTTTACKTTTTATGTCT | 0.269 | 0.270 | 0.151 | 0.150 | 0.203 |
| 7 | 2 | 50244051 | 9309176 | 5147 | AGCATTAAATYAGTCCAGTTG | 0.265 | 0.261 | 0.183 | 0.286 | 0.139 |
| 7 | 2 | 50251406 | 12469244 | 5148 | GATAGGATTCYATGAGGGTGC | 0.030 | 0.030 | 0.150 | 0.222 | 0.168 |
| 7 | 2 | 50258695 | 896685 | 5149 | AGTATTGTAARAAGTCTCTCT | 0.681 | 0.659 | 0.315 | 0.208 | 0.162 |
| 7 | 2 | 50265931 | 985132 | 5150 | CCTCCATTCAYTAGGGAGATA | 0.173 | 0.171 | 0.278 | 0.192 | 0.200 |
| 7 | 2 | 50273114 | 10490240 | 5151 | TTCCCTCACCRTATTTCTAAC | 0.922 | 0.897 | 0.475 | 0.172 | 0.249 |
| 7 | 2 | 50278945 | 17039985 | 5152 | TGAAAGATTGYTTTGTCTCTT | 0.097 | 0.099 | 0.256 | 0.231 | 0.174 |
| 7 | 2 | 50285638 | 10490238 | 5153 | GTCATACTTASGTCAGTCTTC | 0.624 | 0.604 | 0.283 | 0.313 | 0.256 |
| 7 | 2 | 50293510 | 17040013 | 5154 | AGGTATTCTGYAGATATAATT | 0.068 | 0.067 | 0.163 | 0.319 | 0.266 |
| 7 | 2 | 50298927 | 6723207 | 5155 | AGAACTAGTAYATAGTGAGGG | 0.221 | 0.254 | 0.260 | 0.321 | 0.248 |
| 7 | 2 | 50312072 | 12994146 | 5156 | ATGAAAGCAASTTAAGTCTCA | 0.553 | 0.538 | 0.287 | 0.293 | 0.439 |
| 7 | 2 | 50318605 | 1377241 | 5157 | GCTGCTTAGGRGTGCTTAACC | 0.000 | 0.000 | 0.294 | 0.344 | 0.315 |
| 7 | 2 | 50324810 | 17040123 | 5158 | AGTTAAATACYGATTTATAAA | 0.671 | 0.664 | 0.521 | 0.217 | 0.318 |
| 7 | 2 | 50331889 | 4971658 | 5159 | GCACAATAGTRTTGCATTCCT | 0.067 | 0.068 | 0.464 | 0.502 | 0.184 |
| 7 | 2 | 50337287 | 4971659 | 5160 | CCCAACCTCTYGAATGCCCAT | 0.740 | 0.736 | 0.327 | 0.436 | 0.214 |
| 7 | 2 | 50344043 | 1712886 | 5161 | TTATCATCTGMAAAATGAAAA | 0.412 | 0.411 | 0.484 | 0.333 | 0.176 |
| 7 | 2 | 50350726 | 1915221 | 5162 | GTACTCTACAYAAATCTTGCA | 0.115 | 0.115 | 0.468 | 0.252 | 0.239 |
| 7 | 2 | 50353927 | 6724128 | 5163 | ACTTCCTTTAYGTTTTCTTAT | 1.139 | 1.112 | 0.255 | 0.182 | 0.314 |
| 7 | 2 | 50363405 | 17509125 | 5164 | AAATGGAGAAYCAATTTTACC | 0.056 | 0.056 | 0.177 | 0.236 | 0.251 |
| 7 | 2 | 50375352 | 1618655 | 5165 | CAGAATTAAGRGGAATTCACA | 0.164 | 0.182 | 0.259 | 0.198 | 0.209 |
| 7 | 2 | 50388807 | 633128 | 5166 | TAAATAACTTKTCTAATAAAT | 0.133 | 0.133 | 0.061 | 0.266 | 0.141 |
| 7 | 2 | 50395288 | 2682005 | 5167 | CTAAATTTTGRTACTTCAATA | 0.397 | 0.369 | 0.162 | 0.268 | 0.205 |
| 7 | 2 | 50402259 | 1712896 | 5168 | GTAGCTATTARGTCTTCAAAC | 0.302 | 0.306 | 0.307 | 0.092 | 0.131 |
| 7 | 2 | 50405457 | 13030223 | 5169 | TTAGTTGTCARTAACACTGGC | 0.462 | 0.474 | 0.310 | 0.098 | 0.107 |
| 7 | 2 | 50417935 | 7422036 | 5170 | AATACAAATARTGGTGTTAGT | 0.690 | 0.720 | 0.232 | 0.162 | 0.166 |
| 7 | 2 | 50428271 | — | 5171 | ATGAATGTGTRGTATAAAGCT | 0.125 | 0.124 | 0.171 | 0.155 | 0.113 |
| 7 | 2 | 50434710 | 7592085 | 5172 | GTTTATTACASTAATTTCCCT | 0.074 | 0.078 | 0.204 | 0.112 | 0.105 |
| 7 | 2 | 50442046 | 1712891 | 5173 | ATTTTCACATWTCTATGCATG | 0.139 | 0.138 | 0.058 | 0.173 | 0.094 |
| 7 | 2 | 50452128 | 10490229 | 5174 | AATATTGCTCYTCTGAGTGCC | 0.616 | 0.623 | 0.060 | 0.212 | 0.112 |
| 7 | 2 | 50463718 | — | 5175 | CTGGTTGTGASGAAGCTGTGC | 0.116 | 0.117 | 0.201 | 0.089 | 0.098 |
| 7 | 2 | 50479374 | 17040473 | 5176 | AAATATCTCCMGTTACAATAT | 0.142 | 0.133 | 0.385 | 0.084 | 0.099 |
| 7 | 2 | 50492880 | 12475979 | 5177 | TCCAATATAAYTACAGGCAGA | 0.688 | 0.682 | 0.191 | 0.118 | 0.084 |
| 7 | 2 | 50505813 | 4377361 | 5178 | GTACGCCAGCYGAGGCCTCCA | 0.679 | 0.677 | 0.167 | 0.142 | 0.054 |
| 7 | 2 | 50513350 | 4971667 | 5179 | TCCACTGAAGYTACTATTATC | 0.055 | 0.052 | 0.223 | 0.100 | 0.117 |
| 7 | 2 | 50519950 | 1402126 | 5180 | AAAGAGTTCARGTTGAAGAGA | 0.026 | 0.026 | 0.107 | 0.124 | 0.141 |
| 7 | 2 | 50530709 | 10191436 | 5181 | TCTATGTTTCYGACAATAAAA | 0.275 | 0.273 | 0.028 | 0.157 | 0.195 |
| 7 | 2 | 50537843 | 12713098 | 5182 | ATGGATTTTCRAAAGTAAAAC | 0.256 | 0.256 | 0.077 | 0.184 | 0.252 |
| 7 | 2 | 50545936 | 13392422 | 5183 | ATGGCCTCAAYATTCTACTCT | 0.262 | 0.267 | 0.137 | 0.104 | 0.252 |
| 7 | 2 | 50555070 | 1915170 | 5184 | AATAAAATATRCCAAGAAAGG | 0.345 | 0.346 | 0.326 | 0.192 | 0.305 |
| 7 | 2 | 50564676 | 7423850 | 5185 | GTCACTTCACYGGTATTCTTC | 0.218 | 0.225 | 0.329 | 0.410 | 0.225 |
| 7 | 2 | 50571943 | 2160444 | 5186 | TGGAAAGATGWAAAGCTCCTT | 0.819 | 0.825 | 0.404 | 0.388 | 0.133 |
| 7 | 2 | 50578856 | 12613343 | 5187 | AAAGATTTACSGACACTTCAA | 0.179 | 0.194 | 0.651 | 0.427 | 0.144 |
| 7 | 2 | 50584538 | — | 5188 | CAGCAAATGARCAAACGGTAT | 0.514 | 0.510 | 0.618 | 0.394 | 0.302 |
| 7 | 2 | 50594113 | 1005431 | 5189 | TTTACATGGCYATAGCTTTCA | 1.024 | 1.002 | 0.465 | 0.315 | 0.336 |
| 7 | 2 | 50608708 | 17040688 | 5190 | GATTATGTGGRTAAACAACAC | 0.194 | 0.181 | 0.440 | 0.282 | 0.338 |
| 7 | 2 | 50614171 | 17040693 | 5191 | ATTGAGGCCAYGGTAGAAAAG | 0.489 | 0.480 | 0.268 | 0.367 | 0.571 |
| 7 | 2 | 50619827 | 10197380 | 5192 | CTACAAGAGGRGTAAAGGTTA | 0.185 | 0.181 | 0.055 | 0.427 | 0.503 |
| 7 | 2 | 50627282 | 6726707 | 5193 | TGGGCAGCTTYTGGGAGGCAG | 0.037 | 0.035 | 0.291 | 0.371 | 0.505 |
| 7 | 2 | 50634579 | 2241175 | 5194 | TGCTGAAAATKAAATTCTGGT | 0.168 | 0.167 | 0.305 | 0.502 | 0.517 |
| 7 | 2 | 50640296 | 17476739 | 5195 | ACAAAAAGATMATTTCACAGA | 1.132 | 1.149 | 0.345 | 0.456 | 0.544 |
| 7 | 2 | 50647858 | 1014428 | 5196 | ACCTTTACATKATTAATCTGC | 0.473 | 0.472 | 0.962 | 0.382 | 0.471 |
| 7 | 2 | 50655269 | 17536202 | 5197 | GAAGCATAGTKGAGTCACCTT | 0.284 | 0.274 | 0.942 | 0.599 | 0.336 |
| 7 | 2 | 50662806 | — | 5198 | GGAAGAAACARTACTGGAAAT | 1.328 | 1.157 | 0.499 | 0.717 | 0.320 |
| 7 | 2 | 50668252 | 17040792 | 5199 | TCTTGTATGGRTAACACAAAC | 0.027 | 0.027 | 0.694 | 0.723 | 0.233 |
| 7 | 2 | 50675296 | 10175222 | 5200 | TTTTGCCATAKGTAGTAGCGG | 0.264 | 0.254 | 0.757 | 0.460 | 0.285 |
| 7 | 2 | 50681836 | 11125316 | 5201 | TTGCCAAACTSTATATATCAA | 0.875 | 0.853 | 0.187 | 0.361 | 0.281 |
| 7 | 2 | 50696829 | 17040825 | 5202 | GTGGTATCTARCAACAGCGGC | 0.372 | 0.371 | 0.301 | 0.284 | 0.320 |
| 7 | 2 | 50710112 | 12713112 | 5203 | CAGTAAGTCASGAGTTTGAAG | 0.157 | 0.151 | 0.241 | 0.079 | 0.299 |
| 7 | 2 | 50719478 | 11125320 | 5204 | TCAACACATTSTAGATATCTG | 0.364 | 0.330 | 0.038 | 0.076 | 0.248 |
| 7 | 2 | 50727713 | 10179269 | 5205 | TGAGCTCATASAATAAATCCT | 0.117 | 0.118 | 0.055 | 0.096 | 0.361 |
| 7 | 2 | 50735982 | 12713117 | 5206 | TTGTCTTGAGRAGGGACATGG | 0.018 | 0.017 | 0.033 | 0.128 | 0.361 |
| 7 | 2 | 50751973 | 12476492 | 5207 | AACTTCTGCCRAAAACATTAA | 0.480 | 0.473 | 0.038 | 0.109 | 0.403 |
| 7 | 2 | 50764421 | 17040906 | 5208 | ATTGGGCACAMGTACAACTAT | 0.011 | 0.011 | 0.278 | 0.242 | 0.646 |
| 7 | 2 | 50772700 | 10176888 | 5209 | AGGGAAGTTGWTTTAGATGAT | 0.417 | 0.423 | 0.340 | 0.554 | 0.645 |
| 7 | 2 | 50779781 | 13390911 | 5210 | TCCTGAAACASTTTAAGAGAA | 1.037 | 1.024 | 0.498 | 0.596 | 0.858 |
| 7 | 2 | 50788335 | 12713120 | 5211 | CTTAGTGAGAYTGGTTGGTTT | 0.158 | 0.157 | 1.162 | 1.082 | 0.893 |
| 7 | 2 | 50803660 | 858928 | 5212 | ACCTTCTTTGMAGAACAATAC | 0.908 | 0.914 | 1.085 | 1.277 | 0.870 |
| 7 | 2 | 50810606 | 9309186 | 5213 | ATATAATAGTYGTAACTGCAC | 1.522 | 1.552 | 1.301 | 1.763 | 0.876 |
| 7 | 2 | 50821305 | 2194393 | 5214 | TCGTGACTTGSTTGACACACA | 0.268 | 0.268 | 1.662 | 1.705 | 1.561 |
| 7 | 2 | 50831786 | 2352074 | 5215 | TTATCAGTTASTGTGTCAATG | 1.317 | 1.324 | 1.893 | 1.331 | 1.423 |
| 7 | 2 | 50837738 | 10490221 | 5216 | AAAAGTTCTCWCTATATATAC | 0.805 | 0.810 | 1.208 | 1.347 | 2.248 |
| 7 | 2 | 50844593 | 1518548 | 5217 | TAATTCAGGARTTGCCCTTAT | 1.185 | 1.256 | 1.095 | 1.720 | 2.339 |
| 7 | 2 | 50851819 | 858936 | 5218 | AAATTTAAAAYAGCTGAGTGT | 0.244 | 0.233 | 0.564 | 1.078 | 2.763 |
| 7 | 2 | 50858024 | 17040976 | 5219 | ACCTCTTACCRTAATTCCAGT | 0.237 | 0.241 | 1.112 | 1.743 | 2.599 |
| 7 | 2 | 50866998 | — | 5220 | CTCATAATGTMCTTCTGCTAC | 0.118 | 0.124 | 0.588 | 1.829 | 2.564 |
| 7 | 2 | 50876758 | 10184889 | 5221 | CAAAGACACARAGGTTTATCA | 1.801 | 1.960 | 1.453 | 2.450 | 2.705 |
| 7 | 2 | 50883768 | 17502407 | 5222 | GCTTCACATGYTGCCCTCTCA | 0.284 | 0.293 | 2.128 | 2.293 | 2.682 |
| 7 | 2 | 50896377 | 9309194 | 5223 | CACACATGCARTATCTCCCTT | 1.989 | 1.989 | 2.977 | 2.177 | 2.399 |
| 7 | 2 | 50905547 | 9309197 | 5224 | GGGAGGGTCARTTACCAGGAC | 1.352 | 1.357 | 2.243 | 2.498 | 2.297 |
| 7 | 2 | 50914257 | 3850332 | 5225 | GATCTGGAAARTAACTATTAT | 1.828 | 1.766 | 1.974 | 2.630 | 1.780 |
| 7 | 2 | 50922546 | 3850335 | 5226 | CTTTTCCAAGYGTGGGAATAA | 1.000 | 1.007 | 1.930 | 2.088 | 1.869 |
| 7 | 2 | 50933139 | 17568791 | 5227 | GGACATGTTAYGGCTGTGAAA | 0.303 | 0.310 | 1.698 | 2.193 | 1.842 |
| 7 | 2 | 50940313 | 2352077 | 5228 | ATGCGAGGACYAAAGCATTCT | 1.215 | 1.239 | 1.162 | 1.471 | 1.914 |
| 7 | 2 | 50948363 | 9750635 | 5229 | GCTATTAAAAYTCCTGAGATA | 0.892 | 0.870 | 1.040 | 1.286 | 1.346 |
| 7 | 2 | 50959009 | 9309203 | 5230 | GAACTTACTAYCCTTTCTATT | 0.508 | 0.512 | 0.896 | 0.768 | 1.319 |
| 7 | 2 | 50974365 | — | 5231 | TGCCAGCACAYGACATTTGCT | 0.715 | 0.702 | 0.558 | 0.584 | 1.273 |
| 7 | 2 | 50985100 | 17504439 | 5232 | CTCAAAGTGTYAGGTGTAAGG | 0.042 | 0.042 | 0.248 | 0.520 | 1.161 |
| 7 | 2 | 50992132 | 17504614 | 5233 | GAGAAATAATYCTTCATGTCC | 0.468 | 0.500 | 0.217 | 0.269 | 1.618 |
| 7 | 2 | 50998498 | 6710741 | 5234 | TACAGAGTACMCACAGTTCTC | 0.117 | 0.117 | 0.069 | 0.517 | 1.719 |
| 7 | 2 | 51013463 | 2193413 | 5235 | TACTTCCAACRGGAGAATTCT | 0.448 | 0.438 | 0.111 | 0.578 | 1.985 |
| 7 | 2 | 51019436 | 983935 | 5236 | CAATTTGCACRAGGTTAACAG | 0.090 | 0.085 | 0.593 | 1.624 | 2.003 |
| 7 | 2 | 51025393 | 10490172 | 5237 | TTGCTGCTTASAAACAAATTC | 0.296 | 0.282 | 0.831 | 2.248 | 2.616 |
| 7 | 2 | 51032119 | 17041091 | 5238 | GGACAGACAAKTATTTTGTAG | 1.864 | 1.796 | 2.938 | 2.329 | 3.723 |
| 7 | 2 | 51039440 | 17041095 | 5239 | TAGGGCTCTTMAATAGTAGGA | 0.679 | 0.695 | 4.036 | 2.584 | 3.676 |
| 7 | 2 | 51046992 | 1160595 | 5240 | TGTTTCATGTYTTAAAGTGGG | 3.562 | 4.153 | 4.036 | 3.775 | 4.225 |
| 7 | 2 | 51052993 | 1003017 | 5241 | TGGACATACAMTAATGTATCT | 1.320 | 1.479 | 3.511 | 5.267 | 4.568 |
| 7 | 2 | 51058686 | 741421 | 5242 | CTGTCATTCAKTCTAACTTGC | 0.900 | 0.851 | 3.988 | 5.267 | 4.568 |
| 7 | 2 | 51065534 | 7602202 | 5243 | AATATTTAGCYCAATTTCTCT | 1.333 | 1.285 | 3.944 | 4.036 | 5.267 |
| 7 | 2 | 51076203 | 1541602 | 5244 | CTATCAGTAGYATGGGAATGA | 2.030 | 1.966 | 3.481 | 4.153 | 5.267 |
| 7 | 2 | 51083805 | 7579976 | 5245 | CATTCTAACAYCTACAAAGAG | 2.947 | 2.726 | 3.488 | 3.966 | 5.267 |
| 7 | 2 | 51091598 | — | 5246 | ATCTATGCCASATTCCACTGA | 0.074 | 0.074 | 2.730 | 3.966 | 3.869 |
| 7 | 2 | 51096961 | 10205578 | 5247 | TGGAAAGATTKTAATCAGAAA | 1.090 | 1.045 | 2.689 | 3.966 | 3.988 |
| 7 | 2 | 51103966 | 12612704 | 5248 | TATAGATATAYGATATATTGT | 0.000 | 0.000 | 1.609 | 3.225 | 3.422 |
| 7 | 2 | 51107252 | 10174398 | 5249 | TCTGTACCCAYGCTCTCTCCA | 1.535 | 1.429 | 2.171 | 3.153 | 3.364 |
| 7 | 2 | 51117400 | — | 5250 | GCATACAATGYTTGGCTAATG | 0.945 | 0.904 | 2.165 | 2.245 | 3.275 |
| 7 | 2 | 51126792 | 17041161 | 5251 | AGAAAATCCAYAAGTCTAAAT | 1.110 | 1.128 | 2.105 | 2.396 | 3.031 |
| 7 | 2 | 51134758 | 888242 | 5252 | TAAGTATTTAYATAGGTTATC | 0.873 | 0.894 | 1.629 | 2.107 | 2.694 |
| 7 | 2 | 51141925 | 17574007 | 5253 | TTCTATGCAGMCCTCATAAGG | 0.931 | 0.932 | 1.641 | 1.847 | 1.807 |
| 7 | 2 | 51148326 | 17041184 | 5254 | TCAAGTTCCAYTATGCCTGCA | 0.705 | 0.698 | 1.282 | 1.468 | 1.743 |
| 7 | 2 | 51158533 | 12474437 | 5255 | TTTGTGCTCCRTGGATGCAAG | 1.386 | 1.326 | 0.973 | 1.254 | 1.738 |
| 7 | 2 | 51174609 | — | 5256 | ATGGAAATGCMTGCTCACCTA | 0.285 | 0.277 | 0.894 | 1.213 | 1.657 |
| 7 | 2 | 51181930 | — | 5257 | TCCTAGGAGTRTAGGATGAGT | 0.209 | 0.217 | 0.776 | 0.978 | 1.281 |
| 7 | 2 | 51195568 | 6545191 | 5258 | TTTAGAGTTTWTTGTTTTATG | 0.893 | 0.816 | 0.716 | 1.101 | 1.061 |
| 7 | 2 | 51207597 | — | 5259 | AGTACACACASTGACATAATC | 0.408 | 0.402 | 0.594 | 0.995 | 0.884 |
| 7 | 2 | 51219646 | 6545192 | 5260 | AATGGGCTAASTTTCTACTAC | 1.232 | 1.239 | 1.068 | 0.588 | 0.849 |
| 7 | 2 | 51226179 | 17041243 | 5261 | GAAATCTAAGKTTCTCTTTTA | 0.025 | 0.025 | 0.900 | 0.552 | 0.672 |
| 7 | 2 | 51232787 | 1549704 | 5262 | CAGCTGGAATKCACAACGAGA | 1.400 | 1.371 | 0.728 | 0.556 | 0.560 |
| 7 | 2 | 51239401 | 12618610 | 5263 | TTTCTATTAGMCCCTTAAAAA | 0.303 | 0.299 | 0.240 | 0.490 | 0.356 |
| 7 | 2 | 51246851 | — | 5264 | AGGACAAGCTYGTTGGAAGTA | 0.025 | 0.025 | 0.345 | 0.436 | 0.533 |
| 7 | 2 | 51257125 | 4971569 | 5265 | AGGTGCTTTCMTGGGAGTCCC | 0.120 | 0.122 | 0.107 | 0.168 | 0.618 |
| 7 | 2 | 51267562 | 1013164 | 5266 | CACTGGGACTRGGTATGGATC | 0.258 | 0.245 | 0.087 | 0.263 | 0.435 |
| 7 | 2 | 51280190 | 10186486 | 5267 | CCATTTCCCTRGAGATTAAAT | 0.634 | 0.606 | 0.130 | 0.216 | 0.373 |
| 7 | 2 | 51286897 | — | 5268 | GTTATGTATGYGTGTACATGT | 0.260 | 0.257 | 0.216 | 0.271 | 0.246 |
| 7 | 2 | 51292648 | 13404753 | 5269 | GCTAGGAGGARTTAAAATAGC | 0.177 | 0.180 | 0.523 | 0.276 | 0.374 |
| 7 | 2 | 51298389 | 13428293 | 5270 | CAGTTTGCTCKGCTAAGAGGT | 0.441 | 0.431 | 0.522 | 0.272 | 0.170 |
| 7 | 2 | 51306221 | 880384 | 5271 | AGAGGGCCAARGCTTTTTTTC | 1.141 | 1.087 | 0.430 | 0.336 | 0.171 |
| 7 | 2 | 51312996 | 17518964 | 5272 | TTAAATGATTKCAGGTCTTTA | 0.589 | 0.597 | 0.392 | 0.372 | 0.232 |
| 7 | 2 | 51313386 | 17578308 | 5273 | GAAGTTGCTAYGTAGTGTGGC | 0.014 | 0.013 | 0.423 | 0.339 | 0.314 |
| 7 | 2 | 51338825 | 3732051 | 5274 | ACTTAATGCAYCAAAGTAATA | 0.139 | 0.130 | 0.284 | 0.367 | 0.431 |
| 7 | 2 | 51344632 | 12477826 | 5275 | TCTATAATTAMCTCTTGCTGA | 0.484 | 0.478 | 0.133 | 0.378 | 0.363 |
| 7 | 2 | 51350968 | 1468896 | 5276 | TGATCTTTGAKGTTCAAAAAA | 0.710 | 0.755 | 0.220 | 0.234 | 0.374 |
| 7 | 2 | 51356868 | 17041444 | 5277 | TATGCCATCTRCAATGACTGG | 0.110 | 0.113 | 0.334 | 0.325 | 0.539 |
| 7 | 2 | 51362562 | 6745538 | 5278 | AATAATTGGTYCCCTTGTGAC | 0.329 | 0.322 | 0.407 | 0.382 | 0.497 |
| 7 | 2 | 51378902 | 2041947 | 5279 | CTGTATAAACRTATGAAATTA | 0.000 | 0.000 | 0.473 | 0.437 | 0.264 |
| 7 | 2 | 51389580 | 7583309 | 5280 | ATTTAAAATGRTATTACCACT | 0.610 | 0.600 | 0.527 | 0.591 | 0.237 |
| 7 | 2 | 51403035 | 13431262 | 5281 | TACATAGTTAYGCAAACACTA | 0.883 | 0.870 | 0.519 | 0.405 | 0.248 |
| 7 | 2 | 51408733 | 6721829 | 5282 | TTGCAAGCCARGAGGTTCTAG | 0.222 | 0.239 | 0.742 | 0.395 | 0.328 |
| 7 | 2 | 51414723 | 12470385 | 5283 | CAACCCTTCASTAATTCCAAA | 0.341 | 0.336 | 0.541 | 0.425 | 0.708 |
| 8 | 2 | 121573870 | 12477717 | 5284 | AGCCCTGGAGKGTTGGTCTGA | 1.145 | 1.120 | 1.004 | 0.851 | 1.428 |
| 8 | 2 | 121575378 | 17005869 | 5285 | TGTGGGGCTAYGGGCCAGGAA | 1.463 | 1.496 | 1.163 | 0.989 | 1.320 |
| 8 | 2 | 121590453 | 17005870 | 5286 | GAAGCAAATAYGGTAAAATTT | 0.443 | 0.447 | 1.707 | 0.979 | 1.575 |
| 8 | 2 | 121599976 | 10169502 | 5287 | AAACGGGAATRATGGTCAGAC | 0.276 | 0.269 | 1.085 | 1.693 | 1.341 |
| 8 | 2 | 121612456 | 12475346 | 5288 | TGCAGATCACYGAATTTCCCC | 1.048 | 1.010 | 0.486 | 1.687 | 1.418 |
| 8 | 2 | 121618246 | — | 5289 | ACCGTGCAGCYACAGCTCAAA | 0.348 | 0.340 | 0.975 | 1.686 | 1.451 |
| 8 | 2 | 121626028 | 6724707 | 5290 | ATGCTCTTTTYGTGCACCTTT | 0.420 | 0.409 | 1.100 | 1.283 | 1.644 |
| 8 | 2 | 121627437 | 12616919 | 5291 | TTGGACTGTCYGGTTATTGTC | 1.475 | 1.423 | 1.237 | 1.148 | 1.721 |
| 8 | 2 | 121633564 | 10189645 | 5292 | GCTAATGTAAYTTTGGCCACA | 0.657 | 0.658 | 1.452 | 1.205 | 1.278 |
| 8 | 2 | 121646388 | 13015322 | 5293 | CAGGTAAGAAYAAGAAAACCA | 1.479 | 1.528 | 1.452 | 1.060 | 0.816 |
| 8 | 2 | 121654594 | — | 5294 | GAAACAGCATYATCACATACC | 0.000 | 0.000 | 0.873 | 1.224 | 0.814 |
| 8 | 2 | 121664020 | 17179427 | 5295 | GAGCCTTGTGMATGGGCCGGG | 0.191 | 0.185 | 0.783 | 1.071 | 0.890 |
| 8 | 2 | 121674800 | 17006162 | 5296 | AAACACAGGAYCTCTTTTCTT | 0.327 | 0.322 | 0.342 | 0.518 | 0.845 |
| 8 | 2 | 121684644 | 4073806 | 5297 | GTCACCATATRGCAGCTTGCC | 0.000 | 0.000 | 0.178 | 0.458 | 0.838 |
| 8 | 2 | 121692872 | 17794420 | 5298 | CATGCAACCCRAGCGGAAGTG | 0.728 | 0.697 | 0.182 | 0.160 | 0.877 |
| 8 | 2 | 121700701 | 12473888 | 5299 | CAAGTCATGARCCCTGGACAA | 0.027 | 0.027 | 0.206 | 0.248 | 0.526 |
| 8 | 2 | 121710285 | 3768905 | 5300 | CCCCGAGAGCYTGGTCCCACA | 0.143 | 0.147 | 0.241 | 0.279 | 1.162 |
| 8 | 2 | 121723608 | 2119021 | 5301 | AGCTCAGCTCMCTCTCTGGAC | 0.000 | 0.000 | 0.231 | 0.314 | 1.198 |
| 8 | 2 | 121734993 | 17006292 | 5302 | GCCTGATGGAMTATCACCTGC | 0.474 | 0.476 | 0.360 | 0.299 | 1.342 |
| 8 | 2 | 121746144 | 11122852 | 5303 | GCTGAGGTTCWCGCCTACCAG | 0.768 | 0.767 | 0.547 | 1.036 | 1.462 |
| 8 | 2 | 121753101 | 2953083 | 5304 | AGTTGGTAGCMCTTGACCTTC | 0.291 | 0.314 | 0.491 | 1.859 | 1.421 |
| 8 | 2 | 121768706 | — | 5305 | ATGCACTGCASCAGCTCTCCA | 0.000 | 0.000 | 1.621 | 2.055 | 1.765 |
| 8 | 2 | 121775314 | 13028716 | 5306 | TGCCTTTCCAYCTGGACTTAT | 0.380 | 0.381 | 2.154 | 1.930 | 2.827 |
| 8 | 2 | 121794928 | 2580373 | 5307 | AACTGTGCCAYCTCATTGTCC | 2.612 | 2.642 | 2.390 | 1.746 | 2.636 |
| 8 | 2 | 121820468 | 12622908 | 5308 | CTGTTGCCACYACTGATGCCC | 1.503 | 1.574 | 2.247 | 2.043 | 2.476 |
| 8 | 2 | 121833968 | 12479320 | 5309 | TGGTGCCAATRTCTCTAAAAG | 0.910 | 0.883 | 1.897 | 3.093 | 2.476 |
| 8 | 2 | 121834342 | 17006394 | 5310 | CCTAAAAAATRGCAAGAAGGA | 0.385 | 0.395 | 1.392 | 2.712 | 2.449 |
| 8 | 2 | 121859795 | 6716602 | 5311 | CAGAGGGAAAYTTGAGGGAGT | 0.275 | 0.270 | 2.539 | 2.510 | 2.203 |
| 8 | 2 | 121870433 | 10496566 | 5312 | ATATAGAATAWGTTTCAGCCC | 1.420 | 1.483 | 2.237 | 2.010 | 2.204 |
| 8 | 2 | 121886006 | 10191223 | 5313 | ACCACAGAAAKGGAGAAAAAT | 4.004 | 4.153 | 2.200 | 1.819 | 2.021 |
| 8 | 2 | 121891735 | 10496567 | 5314 | CTACGTCTAAKTATTATAAAA | 0.206 | 0.207 | 2.677 | 1.574 | 2.096 |
| 8 | 2 | 121897370 | 10496568 | 5315 | AACAGACATCMTATAAACTTA | 0.394 | 0.401 | 2.153 | 1.799 | 1.718 |
| 8 | 2 | 121911303 | 756504 | 5316 | TTGTTTGTGTSCTGTGGACAC | 0.000 | 0.000 | 0.183 | 1.718 | 1.730 |
| 8 | 2 | 121923785 | 17006485 | 5317 | TATCAATGCAYTGCCCAAGAG | 0.438 | 0.414 | 0.421 | 1.667 | 1.631 |
| 8 | 2 | 121930788 | 17006497 | 5318 | ACGACTTGCTRGAAAGAGGGT | 0.228 | 0.224 | 0.324 | 0.593 | 2.019 |
| 8 | 2 | 121936188 | 2118387 | 5319 | TTGCTCCAACRTTTTCTGGTC | 0.761 | 0.773 | 0.823 | 1.071 | 2.097 |
| 8 | 2 | 121950085 | 17039719 | 5320 | CTCCACTGAASTCCCTAAGAA | 0.162 | 0.164 | 0.790 | 1.212 | 1.815 |
| 8 | 2 | 121958256 | 17006551 | 5321 | ACATCCCATGSTCCCCTTTGT | 1.661 | 1.585 | 1.488 | 1.531 | 0.947 |
| 8 | 2 | 121964620 | — | 5322 | GGAAACACTTYCCTAAGATAC | 0.366 | 0.318 | 1.551 | 1.391 | 0.909 |
| 8 | 2 | 121989999 | 6730694 | 5323 | GTACAGGCCAYATGAAGTCCC | 1.813 | 1.761 | 2.134 | 1.540 | 0.873 |
| 8 | 2 | 121994411 | 7608301 | 5324 | ACCAAACTCARCAAGAGGGCC | 0.752 | 0.737 | 1.549 | 1.291 | 0.795 |
| 8 | 2 | 122017375 | 2164797 | 5325 | AGCCCCCAGCSCGCCAACAGG | 1.546 | 1.546 | 1.690 | 1.212 | 0.740 |
| 8 | 2 | 122041926 | 4848712 | 5326 | TTCATCCACTYGTCTAGAGGG | 0.026 | 0.027 | 0.693 | 0.767 | 0.752 |
| 8 | 2 | 122042281 | — | 5327 | AATGAAACAARCATTTCTTTA | 0.593 | 0.591 | 0.420 | 0.733 | 0.648 |
| 8 | 2 | 122065350 | 10496574 | 5328 | CAGTACTAAAKTCCTATACTA | 0.062 | 0.067 | 0.037 | 0.299 | 0.628 |
| 8 | 2 | 122070442 | 10496573 | 5329 | AGTCTACCAARGAAGTGCTAA | 0.103 | 0.103 | 0.068 | 0.192 | 0.321 |
| 8 | 2 | 122093087 | 287790 | 5330 | TCTTATCTTTRTGCTTTCCTG | 0.139 | 0.143 | 0.018 | 0.020 | 0.315 |
| 8 | 2 | 122102558 | 10185512 | 5331 | TAAAAAGTAGYAATACATAGG | 0.224 | 0.230 | 0.033 | 0.024 | 0.138 |
| 8 | 2 | 122108669 | 192608 | 5332 | CTTCTGTCAARCTTTTTAGAC | 0.165 | 0.168 | 0.036 | 0.004 | 0.125 |
| 8 | 2 | 122119204 | 6541792 | 5333 | TGCCTGCAGTSTGACACTGAT | 0.241 | 0.249 | 0.035 | 0.009 | 0.051 |
| 8 | 2 | 122132877 | — | 5334 | CAGCAAAACTWTTTATACATC | 0.214 | 0.215 | 0.017 | 0.025 | 0.073 |
| 8 | 2 | 122138380 | 6541794 | 5335 | TTTCTTATCARTGGGCCCTCT | 0.093 | 0.096 | 0.035 | 0.061 | 0.122 |
| 8 | 2 | 122165738 | 17833009 | 5336 | AGAATTCATAYACAAACCCCA | 0.037 | 0.037 | 0.078 | 0.138 | 0.159 |
| 8 | 2 | 122184058 | 17039745 | 5337 | ATAATTTCCTYCTTTCCAATG | 0.277 | 0.274 | 0.179 | 0.177 | 0.251 |
| 8 | 2 | 122191938 | — | 5338 | ATAGTGGTAGWTTCTGTCCTG | 0.512 | 0.507 | 0.372 | 0.392 | 0.326 |
| 8 | 2 | 122206162 | 6726677 | 5339 | TCACATATTCYGGGGACTGTT | 0.581 | 0.573 | 0.546 | 0.471 | 0.390 |
| 8 | 2 | 122224032 | 287803 | 5340 | AGTGCTAACASGTCAATAATA | 0.773 | 0.771 | 0.998 | 0.661 | 0.481 |
| 8 | 2 | 122232697 | — | 5341 | GCTTACAACTMGGCAAACATC | 0.000 | 0.000 | 1.059 | 0.846 | 0.559 |
| 8 | 2 | 122238287 | 7565841 | 5342 | CGTATGTTGASAGTGCACTAA | 1.103 | 1.112 | 1.048 | 0.949 | 0.547 |
| 8 | 2 | 122263080 | 17006811 | 5343 | AAGATTGAAARTCACTTATGA | 0.000 | 0.000 | 0.973 | 0.935 | 0.563 |
| 8 | 2 | 122273164 | 4848159 | 5344 | GCATGAGCATYTTTCATTGTC | 0.760 | 0.746 | 0.955 | 0.982 | 0.653 |
| 8 | 2 | 122285711 | 4848719 | 5345 | CCATGGCCTTRGCCTGTCTGC | 0.564 | 0.573 | 0.694 | 0.794 | 0.572 |
| 8 | 2 | 122311345 | 6761861 | 5346 | AACGATGACCSAGAGGCTCTT | 0.559 | 0.553 | 0.720 | 0.669 | 0.687 |
| 8 | 2 | 122315106 | 6541810 | 5347 | CACACAGGCTSTTTAAGGTAA | 0.565 | 0.587 | 0.484 | 0.428 | 0.562 |
| 8 | 2 | 122336423 | 4430963 | 5348 | GGCTGAGTAAMAATCTTTTTG | 0.664 | 0.591 | 0.314 | 0.329 | 0.421 |
| 8 | 2 | 122352090 | 4254513 | 5349 | CAGAATTCTARGTTGGTGGTG | 0.135 | 0.140 | 0.267 | 0.393 | 0.612 |
| 9 | 2 | 127494872 | 17014773 | 5350 | GAATTCTTTGSTGTAAAGCTT | 0.488 | 0.454 | 0.754 | 0.234 | 0.962 |
| 9 | 2 | 127507108 | 11893989 | 5351 | GTATTTGATCRAATCTCTCAT | 1.817 | 1.824 | 0.552 | 0.385 | 1.199 |
| 9 | 2 | 127513886 | 11902275 | 5352 | GGACCACAGCMTGGCAGAGGA | 0.248 | 0.246 | 0.616 | 0.383 | 0.781 |
| 9 | 2 | 127526927 | 17014818 | 5353 | GACCGGCCCAYTTCTGTCTGG | 0.106 | 0.108 | 0.701 | 0.744 | 0.754 |
| 9 | 2 | 127536416 | 17014851 | 5354 | CAGGCACGAGMGGAGGTTTAT | 0.162 | 0.160 | 0.073 | 1.289 | 1.256 |
| 9 | 2 | 127542763 | 17844995 | 5355 | TCTTTTTGGCRGTTTGAAGGG | 0.628 | 0.659 | 0.592 | 1.379 | 1.247 |
| 9 | 2 | 127549063 | 6754017 | 5356 | TTGCATAGATSTCTGTCAAAC | 0.019 | 0.019 | 1.459 | 0.841 | 1.058 |
| 9 | 2 | 127553271 | 13430599 | 5357 | CCTCACACCGKGTCCAGGCTG | 1.786 | 1.860 | 1.642 | 1.390 | 1.355 |
| 9 | 2 | 127559423 | 13425613 | 5358 | GAGGAAGGAGMCCGAAAGAAA | 1.569 | 1.611 | 1.341 | 1.402 | 1.332 |
| 9 | 2 | 127594668 | 4663100 | 5359 | TTCCCGTTTGSGGTTGGGGAC | 0.645 | 0.637 | 2.190 | 1.338 | 0.835 |
| 9 | 2 | 127606162 | 6710467 | 5360 | ATCCGAAGGTRAGTGGTATTT | 0.132 | 0.136 | 1.258 | 1.478 | 0.848 |
| 9 | 2 | 127613550 | 3943703 | 5361 | CATTATTTTAYTTTCCCTTAT | 1.556 | 1.582 | 0.493 | 1.680 | 0.842 |
| 9 | 2 | 127620718 | 10208217 | 5362 | CTAATACTTASGTGTACATCA | 0.087 | 0.083 | 0.630 | 0.896 | 1.364 |
| 9 | 2 | 127630065 | 729666 | 5363 | TCACTCTGCTYTTTGGAATGT | 0.064 | 0.065 | 0.742 | 0.454 | 1.745 |
| 9 | 2 | 127638462 | 17701884 | 5364 | TGCTAAACATRAGATACTCAG | 0.931 | 0.962 | 0.186 | 0.283 | 2.625 |
| 9 | 2 | 127644972 | 17015008 | 5365 | AGCTGAAAGGRCTCTGAATGT | 0.381 | 0.386 | 0.282 | 0.732 | 3.364 |
| 9 | 2 | 127650421 | 4662708 | 5366 | AGTCGGCAGAKGCTCTGAGAT | 0.161 | 0.164 | 0.253 | 0.798 | 3.735 |
| 9 | 2 | 127668493 | 13006847 | 5367 | GCTGAACAGGYCAAACTATTT | 0.277 | 0.260 | 0.494 | 1.511 | 3.551 |
| 9 | 2 | 127692604 | 6710496 | 5368 | AAGCCCCCATMCCAGAAATAA | 0.050 | 0.068 | 1.208 | 3.267 | 3.775 |
| 9 | 2 | 127723916 | 6430936 | 5369 | CCCGAACAGGMTTTTGTTCAC | 1.597 | 1.809 | 2.784 | 4.187 | 3.403 |
| 9 | 2 | 127729734 | 4662717 | 5370 | GTTCCTGGGGYTTGCACTGAT | 1.757 | 1.935 | 4.187 | 4.062 | 3.488 |
| 9 | 2 | 127749557 | 4150474 | 5371 | ACAAACCCACMAAGAAAACAG | 2.034 | 2.101 | 4.789 | 4.312 | 3.568 |
| 9 | 2 | 127754774 | 4150454 | 5372 | CCATCACTTTYAGACCTGTCC | 4.078 | 4.488 | 4.312 | 4.153 | 3.735 |
| 9 | 2 | 127776393 | 4233584 | 5373 | CCTGGATTTCYTACTCACTGT | 4.250 | 3.804 | 4.568 | 4.036 | 3.735 |
| 9 | 2 | 127802584 | 12613413 | 5374 | TTTTGATGGTYCACATGCCAA | 0.498 | 0.536 | 4.153 | 3.775 | 3.761 |
| 9 | 2 | 127810283 | 17015199 | 5375 | TTTTCTTTCTRCTTCCCAAAG | 2.959 | 2.899 | 2.959 | 3.723 | 3.698 |
| 9 | 2 | 127828431 | 6714840 | 5376 | GCAAAGGATCWGTTTCCAAGT | 1.018 | 1.018 | 1.933 | 3.835 | 3.944 |
| 9 | 2 | 127831525 | 6727155 | 5377 | TTTAAAGGGCWAAGGCTATTC | 0.731 | 0.715 | 1.960 | 2.972 | 3.467 |
| 9 | 2 | 127862992 | 2276683 | 5378 | AAACACAGAGSTCAGTGGAAA | 0.386 | 0.393 | 1.160 | 1.860 | 2.951 |
| 9 | 2 | 127880407 | 7599210 | 5379 | TCCTTGGCCARGCATACTGGC | 0.000 | 0.000 | 0.823 | 1.794 | 2.448 |
| 9 | 2 | 127886129 | 6753288 | 5380 | ATTATAGCGCRCTTAAGTTAC | 1.310 | 1.269 | 0.745 | 0.722 | 1.996 |
| 9 | 2 | 127906285 | 1568277 | 5381 | AAGAGAAACAYGGATGGAGCG | 0.247 | 0.251 | 0.750 | 0.526 | 1.315 |
| 9 | 2 | 127912750 | 12478656 | 5382 | TCATGGGCAGKAAACTGTTCT | 0.587 | 0.586 | 0.536 | 0.377 | 1.299 |
| 9 | 2 | 127930463 | 17015274 | 5383 | GTGCTTAGATRTGGAGAGCAT | 0.377 | 0.384 | 0.221 | 0.603 | 0.723 |
| 9 | 2 | 127937786 | 3889307 | 5384 | CGGTCTCAACYTGGTTTTGTT | 0.017 | 0.017 | 0.165 | 0.720 | 0.642 |
| 9 | 2 | 127945128 | 10496663 | 5385 | ATGACAAGTGYTATATGTTAG | 0.299 | 0.304 | 0.399 | 0.442 | 0.748 |
| 9 | 2 | 127955157 | 777557 | 5386 | TTAGAAACGCRTATTAAACTC | 0.112 | 0.110 | 0.581 | 0.433 | 0.676 |
| 9 | 2 | 127963870 | 334156 | 5387 | CCTTGAAGAGYCCATTATTTC | 1.292 | 1.359 | 0.662 | 0.441 | 0.682 |
| 9 | 2 | 127971045 | 11681225 | 5388 | CCCGGAACTTKTCATGCACTA | 0.932 | 0.970 | 0.652 | 0.677 | 0.475 |
| 9 | 2 | 127982848 | — | 5389 | CACGAAAACAYAGCATTATGT | 0.209 | 0.218 | 0.953 | 0.705 | 0.589 |
| 9 | 2 | 127988904 | 334137 | 5390 | CAGTTTAATCRCTCATTTTCT | 0.292 | 0.285 | 0.917 | 0.794 | 1.068 |
| 9 | 2 | 128006344 | 11684705 | 5391 | TTCAAGGTTASCTGAATATTT | 0.633 | 0.642 | 0.458 | 0.873 | 1.011 |
| 9 | 2 | 128015247 | 2052954 | 5392 | GTGAACGTCAYTACAAAGTGC | 1.193 | 1.233 | 0.587 | 0.688 | 1.310 |
| 9 | 2 | 128023821 | 17015401 | 5393 | ATAGCTCCTGKTACTTTTGGC | 0.030 | 0.031 | 0.581 | 1.356 | 1.335 |
| 9 | 2 | 128033130 | 11688201 | 5394 | GCTTAAAGTTRTGTCTATAGC | 0.536 | 0.569 | 0.678 | 1.209 | 1.618 |
| 9 | 2 | 128040262 | — | 5395 | ATCCCATCTTYCCAGAGGAGA | 0.258 | 0.258 | 1.269 | 1.394 | 1.181 |
| 9 | 2 | 128057833 | 10928772 | 5396 | ATGCAGCTGTKTGCAGATGGC | 0.861 | 0.822 | 1.262 | 1.237 | 0.907 |
| 10 | 2 | 162067733 | 10193429 | 5397 | TGTGGAAATAYTGTTGTAAAA | 0.218 | 0.222 | 0.178 | 0.176 | 0.084 |
| 10 | 2 | 162078126 | 12463483 | 5398 | TTTCAAGCTGKCCAACACCCA | 0.292 | 0.292 | 0.172 | 0.166 | 0.079 |
| 10 | 2 | 162084212 | 2216941 | 5399 | AGTCTTTCCCWTTCAAATCAA | 0.138 | 0.123 | 0.248 | 0.178 | 0.126 |
| 10 | 2 | 162090777 | 16845844 | 5400 | TCTGGTCATCRTTATTTTTGG | 0.211 | 0.218 | 0.233 | 0.190 | 0.091 |
| 10 | 2 | 162114653 | 12470386 | 5401 | TTACTCTTCAYCTAAGTTTAG | 0.807 | 1.157 | 0.159 | 0.088 | 0.105 |
| 10 | 2 | 162122423 | 918964 | 5402 | ATTGATTTCCRTGAGCTGAGC | 0.182 | 0.178 | 0.244 | 0.145 | 0.203 |
| 10 | 2 | 162130415 | 963758 | 5403 | ATTTGGAGCAYTTGCCAGAGC | 0.057 | 0.056 | 0.205 | 0.131 | 0.424 |
| 10 | 2 | 162147459 | 6742389 | 5404 | ATATTTTGTAYGATTCTAATT | 0.000 | 0.000 | 0.110 | 0.182 | 0.387 |
| 10 | 2 | 162156767 | 994383 | 5405 | ATCCTAATCCRTCATCTTCTA | 0.089 | 0.089 | 0.111 | 0.315 | 0.681 |
| 10 | 2 | 162174354 | 6731620 | 5406 | TAACTTTCTGYTGAAATGAAA | 0.625 | 0.591 | 0.194 | 0.489 | 0.622 |
| 10 | 2 | 162176411 | 3849351 | 5407 | CAGAATTACCRTAAGACAGGC | 0.166 | 0.169 | 0.334 | 0.543 | 0.713 |
| 10 | 2 | 162200351 | 3914093 | 5408 | CATTAATTTCRGAGAAATTGT | 0.000 | 0.000 | 1.042 | 1.095 | 0.664 |
| 10 | 2 | 162237504 | 13410944 | 5409 | ACTTTTAGGTYTCAGGAAATT | 0.779 | 0.780 | 0.924 | 0.900 | 0.553 |
| 10 | 2 | 162264719 | — | 5410 | AGCAGACTAASAAAAATTTAT | 1.571 | 1.539 | 1.761 | 1.093 | 0.640 |
| 10 | 2 | 162270121 | 971027 | 5411 | ACACTTAGACWGATTCCATGT | 0.360 | 0.368 | 1.391 | 0.910 | 0.614 |
| 10 | 2 | 162283074 | 16845940 | 5412 | AGGTTTGACARATGCTGGCTT | 1.720 | 1.820 | 1.354 | 1.114 | 0.547 |
| 10 | 2 | 162298795 | — | 5413 | GTCAGAGTTTRTTGGAATTTT | 0.015 | 0.015 | 0.559 | 1.120 | 0.593 |
| 10 | 2 | 162310868 | — | 5414 | TATTAATATTRTGTTTATGAT | 0.000 | 0.000 | 0.644 | 0.762 | 0.575 |
| 10 | 2 | 162330250 | 10803781 | 5415 | GTGAAATGGCRGCATTCTCTT | 0.005 | 0.005 | 0.043 | 0.339 | 0.545 |
| 10 | 2 | 162343872 | 2389424 | 5416 | ATTTTACTATMAATGAATATT | 0.000 | 0.000 | 0.042 | 0.293 | 0.615 |
| 10 | 2 | 162354105 | — | 5417 | ACGTAGGGTASATGGTGGGAT | 0.279 | 0.555 | 0.038 | 0.032 | 0.845 |
| 10 | 2 | 162365216 | 9677118 | 5418 | CAGCTTGAGTWATAGTTTGAA | 0.004 | 0.004 | 0.114 | 0.043 | 0.625 |
| 10 | 2 | 162374606 | 1567981 | 5419 | TTGAGTGTTARCGTTGCTAAT | 0.181 | 0.183 | 0.137 | 0.115 | 0.575 |
| 10 | 2 | 162383186 | 7578900 | 5420 | AATTGGTAGTSTGCCTCCATA | 0.284 | 0.280 | 0.061 | 0.606 | 0.224 |
| 10 | 2 | 162393175 | 17728078 | 5421 | TAAACTGTGARTCAGTAAAAT | 0.461 | 0.457 | 0.248 | 0.584 | 0.227 |
| 10 | 2 | 162407761 | 4664048 | 5422 | AATTTTCCTAWTGTATTGGCT | 0.050 | 0.051 | 1.019 | 0.473 | 0.248 |
| 10 | 2 | 162412390 | 16846076 | 5423 | ATTTTGGTTTYATTTGTCCAT | 0.845 | 0.843 | 1.052 | 0.475 | 0.321 |
| 10 | 2 | 162434172 | 971282 | 5424 | AAAAAGGAAGYACATCTTGTG | 1.929 | 1.967 | 0.899 | 0.450 | 0.321 |
| 10 | 2 | 162444883 | 7609393 | 5425 | CACTTAAAAAMCTAGCTCTTA | 0.460 | 0.448 | 0.901 | 0.519 | 0.240 |
| 10 | 2 | 162454850 | 10193728 | 5426 | AAGATGCTATYGAGGGTTCTG | 0.185 | 0.181 | 0.556 | 0.517 | 0.243 |
| 10 | 2 | 162498178 | 7608076 | 5427 | AGTGAGAATAYATGACATTTC | 0.000 | 0.000 | 0.099 | 0.653 | 0.312 |
| 10 | 2 | 162512676 | 1710513 | 5428 | ATAAAATTTTWAAAAAAAGCC | 0.000 | 0.000 | 0.094 | 0.403 | 0.328 |
| 10 | 2 | 162525680 | 1227925 | 5429 | ATCTAAAAACRCTAATTTTTG | 0.534 | 0.523 | 0.133 | 0.036 | 0.301 |
| 10 | 2 | 162536523 | 1227936 | 5430 | AACAGGCCCCWAATTATGCTA | 0.446 | 0.419 | 0.151 | 0.055 | 0.317 |
| 10 | 2 | 162542388 | 1227932 | 5431 | AATTAAAAATMATAAACATAT | 0.000 | 0.000 | 0.126 | 0.075 | 0.175 |
| 10 | 2 | 162550936 | 10179404 | 5432 | ACAGAAATAAYTGAACATTGC | 0.044 | 0.043 | 0.140 | 0.122 | 0.020 |
| 10 | 2 | 162557433 | 11683174 | 5433 | AAAAAGGTCTYTATTCATCTT | 0.022 | 0.024 | 0.107 | 0.118 | 0.059 |
| 10 | 2 | 162563221 | 6707956 | 5434 | ATAGTAGAAAKGTCAGAAAAT | 0.632 | 0.590 | 0.108 | 0.046 | 0.240 |
| 10 | 2 | 162568431 | 16846177 | 5435 | TGTTGGTTCARTCTGAATCTT | 0.327 | 0.319 | 0.112 | 0.023 | 0.631 |
| 10 | 2 | 162578850 | 17729070 | 5436 | ACTAATCAAASAGTGCCATAT | 0.265 | 0.273 | 0.106 | 0.108 | 0.978 |
| 10 | 2 | 162586481 | 7594396 | 5437 | TTGTCTCACARGAGGTCCATT | 0.131 | 0.133 | 0.023 | 0.493 | 1.313 |
| 10 | 2 | 162605465 | 6432705 | 5438 | ACAATCTCTAYTTACTTATGT | 0.077 | 0.073 | 0.197 | 1.106 | 1.720 |
| 10 | 2 | 162610830 | 10930036 | 5439 | ATATGCCATGYGTATAGCAAC | 0.035 | 0.036 | 0.760 | 1.332 | 2.008 |
| 10 | 2 | 162628500 | 12474587 | 5440 | GTTCATATTTKTCTCCAAGAT | 1.212 | 1.159 | 1.358 | 1.665 | 2.106 |
| 10 | 2 | 162636693 | 4637136 | 5441 | TTTGGTTTACRTTAAATCAAG | 1.717 | 1.711 | 1.747 | 2.129 | 2.277 |
| 10 | 2 | 162644128 | 4500960 | 5442 | TCCAGCAGCAYGTTACTGTCT | 1.911 | 1.835 | 2.098 | 2.442 | 2.361 |
| 10 | 2 | 162653508 | 4664442 | 5443 | CTTTTAATGGRTCCTATGTAA | 1.537 | 1.515 | 2.562 | 2.350 | 2.299 |
| 10 | 2 | 162657263 | 4295021 | 5444 | CCTGTTGATTKTTTAGCTGAA | 2.045 | 2.188 | 3.102 | 2.531 | 2.369 |
| 10 | 2 | 162671072 | 1861979 | 5445 | TTAAAGCCTGYAAGCACCAAA | 2.474 | 2.222 | 2.983 | 2.795 | 2.451 |
| 10 | 2 | 162680415 | 2287509 | 5446 | CCTCCTTGTTKCTCTCCAAAT | 2.232 | 2.264 | 2.916 | 2.905 | 2.655 |
| 10 | 2 | 162688346 | — | 5447 | CTTTTGGAGAYCCCTAACAAT | 0.394 | 0.395 | 2.740 | 2.784 | 2.683 |
| 10 | 2 | 162694710 | 1014445 | 5448 | TCTGTAAAGCRCTCTCATTTC | 1.404 | 1.396 | 1.830 | 2.596 | 2.755 |
| 10 | 2 | 162702535 | 12692646 | 5449 | GATTTCCACTWCAAGTTGGTT | 1.488 | 1.513 | 1.425 | 2.543 | 2.914 |
| 10 | 2 | 162712470 | 2284873 | 5450 | GGATATGGCCYAACAGGAAAA | 0.038 | 0.039 | 1.570 | 2.023 | 2.779 |
| 10 | 2 | 162726834 | 16822665 | 5451 | TTGATGGAATYTTTACGATCT | 1.562 | 1.526 | 1.662 | 1.446 | 2.399 |
| 10 | 2 | 162741920 | 10490422 | 5452 | CATTCTTATGKTGTTCTTCTA | 0.779 | 0.793 | 1.227 | 1.574 | 2.380 |
| 10 | 2 | 162749424 | 12469968 | 5453 | CTCTACCTCARTTATACATCC | 1.477 | 1.503 | 1.267 | 1.178 | 1.731 |
| 10 | 2 | 162764974 | 10930042 | 5454 | TATCTTCACCRTCTGTTTCTG | 0.154 | 0.142 | 0.834 | 0.735 | 1.187 |
| 10 | 2 | 162777856 | 4420712 | 5455 | GGAAAGATTAKATTTGAGCCA | 0.253 | 0.222 | 0.519 | 1.282 | 1.080 |
| 10 | 2 | 162789104 | 16846291 | 5456 | AGGCACTTCAKTTCAAAAATC | 0.660 | 0.621 | 0.105 | 0.723 | 1.083 |
| 10 | 2 | 162797892 | 10185151 | 5457 | CCTGTCTCTARGTCATGCATT | 0.066 | 0.068 | 0.549 | 0.475 | 0.720 |
| 10 | 2 | 162807953 | 10490423 | 5458 | GCACATGAATYGATATAAAGA | 0.238 | 0.235 | 0.483 | 0.129 | 0.849 |
| 10 | 2 | 162824895 | 6732914 | 5459 | CAGACAATGGYAAAGAAGCTT | 1.503 | 1.538 | 0.272 | 0.395 | 0.648 |
| 10 | 2 | 162835518 | 13001107 | 5460 | CTACTTGAAGSCCACAGAGAT | 0.054 | 0.052 | 0.246 | 0.356 | 0.729 |
| 10 | 2 | 162839680 | 4664450 | 5461 | TATGCCCAGTKTTCTGGTGAA | 0.032 | 0.033 | 0.675 | 0.370 | 0.445 |
| 10 | 2 | 162857841 | 6707966 | 5462 | TAAGGTTGCAYTGCCTCGTGA | 0.028 | 0.027 | 0.157 | 0.536 | 0.461 |
| 10 | 2 | 162869862 | 6715552 | 5463 | TTTTCCTGAARATCATCATTA | 1.359 | 1.378 | 0.349 | 0.815 | 0.424 |
| 10 | 2 | 162880231 | 4664452 | 5464 | AAAGGTTCATYTTCTATCCCC | 0.071 | 0.068 | 0.580 | 0.449 | 0.333 |
| 10 | 2 | 162888686 | 6706926 | 5465 | GAAGCACTAGKTTCCTTTGAT | 0.645 | 0.639 | 1.122 | 0.493 | 0.360 |
| 10 | 2 | 162898589 | 16846387 | 5466 | TTTATACTTTYCTTGTTTTGT | 0.735 | 0.688 | 0.614 | 0.478 | 0.332 |
| 10 | 2 | 162906489 | 16846391 | 5467 | ATACCTTTGARTAATCACTTT | 1.196 | 1.201 | 0.696 | 0.497 | 0.183 |
| 10 | 2 | 162911936 | 3788970 | 5468 | GCCATAATGARAATAATCCTC | 0.207 | 0.198 | 0.444 | 0.244 | 0.188 |
| 10 | 2 | 162920558 | — | 5469 | CAAACTTGATKTCTTTATCTT | 0.256 | 0.251 | 0.238 | 0.230 | 0.299 |
| 10 | 2 | 162928735 | 16846429 | 5470 | CCAGTGTTACYACCTTACATT | 0.020 | 0.020 | 0.018 | 0.196 | 0.382 |
| 10 | 2 | 162934353 | 1125605 | 5471 | CATATCGTCAYATCATACTGT | 0.095 | 0.095 | 0.008 | 0.101 | 0.171 |
| 10 | 2 | 162940042 | 16846499 | 5472 | TTTTTTTATCWACTATGCAAA | 0.205 | 0.204 | 0.031 | 0.048 | 0.221 |
| 10 | 2 | 162946354 | 6755575 | 5473 | TTGTGAACTGYACTAATTAAT | 0.075 | 0.077 | 0.063 | 0.081 | 0.145 |
| 10 | 2 | 162954331 | 3747517 | 5474 | AACTGTCTCAYGTTCGATAAC | 0.546 | 0.536 | 0.199 | 0.055 | 0.188 |
| 10 | 2 | 162960189 | 4664053 | 5475 | TAAACATGGGWTAAACTAAGT | 0.114 | 0.131 | 0.312 | 0.119 | 0.063 |
| 10 | 2 | 162966835 | 10179671 | 5476 | GACAAGATCTRCATTTGATTT | 0.778 | 0.746 | 0.288 | 0.115 | 0.113 |
| 10 | 2 | 162973735 | 6758639 | 5477 | TGCAAATTCTRTAATACTTTT | 0.567 | 0.574 | 0.278 | 0.274 | 0.296 |
| 10 | 2 | 162980587 | 2163215 | 5478 | TAATCCTCACKCCTGGAAGGT | 0.012 | 0.012 | 0.276 | 0.275 | 0.368 |
| 10 | 2 | 162989298 | 12478709 | 5479 | CTGACCGTCAYGTTTAGGAAG | 0.510 | 0.495 | 0.357 | 0.342 | 0.688 |
| 10 | 2 | 163003491 | 12468353 | 5480 | AGTTTAGTGARAAACCCTGGA | 0.107 | 0.104 | 0.188 | 0.761 | 0.665 |
| 11 | 2 | 198533144 | 3851979 | 5481 | TTTGCTAGGCWGTAAGGAATA | 3.455 | 3.551 | 3.241 | 2.210 | 1.583 |
| 11 | 2 | 198538737 | 6707521 | 5482 | TGAAGACAAGRACTAGGCCGA | 0.000 | 0.000 | 2.149 | 1.954 | 1.124 |
| 11 | 2 | 198546560 | 700686 | 5483 | TAGAAGTCGARATAAGTTAGA | 0.082 | 0.083 | 2.141 | 2.240 | 1.112 |
| 11 | 2 | 198554006 | 700692 | 5484 | CTTGAAGTCAYCCCGTGTATA | 0.227 | 0.224 | 0.243 | 1.372 | 1.235 |
| 11 | 2 | 198561218 | — | 5485 | TTTTCTGGATRTTAAACTACA | 1.052 | 1.043 | 0.217 | 1.087 | 1.303 |
| 11 | 2 | 198569830 | 9288280 | 5486 | AACCCTGAGGRAAATTTAATA | 0.015 | 0.015 | 0.300 | 0.138 | 1.397 |
| 11 | 2 | 198577143 | 2060488 | 5487 | CCTTAGGTTTKTATATTCTGT | 0.275 | 0.286 | 0.387 | 0.127 | 0.886 |
| 11 | 2 | 198585479 | 6734781 | 5488 | TCCTCCATTCRTCTCCATTTC | 0.296 | 0.301 | 0.040 | 0.171 | 0.739 |
| 11 | 2 | 198593672 | 6760891 | 5489 | CAAAAATCTAKGTATCTTCCT | 0.000 | 0.000 | 0.079 | 0.199 | 0.581 |
| 11 | 2 | 198602052 | 4241195 | 5490 | CTTTTGTCCCRAGTTCCAGTT | 0.006 | 0.006 | 0.076 | 0.052 | 1.012 |
| 11 | 2 | 198617826 | 1401092 | 5491 | ATTACATAAGMCAACTCACAG | 0.257 | 0.274 | 0.080 | 0.217 | 1.437 |
| 11 | 2 | 198624136 | 1464211 | 5492 | CAGTATTTAARTTACTGAGCT | 0.000 | 0.000 | 0.062 | 0.815 | 1.458 |
| 11 | 2 | 198631344 | 11887138 | 5493 | AAACTAGACAWCTATACCAAT | 0.261 | 0.273 | 0.373 | 1.782 | 1.657 |
| 11 | 2 | 198644132 | — | 5494 | TCCTGAGTAGWCACCAATGCC | 0.169 | 0.175 | 1.407 | 2.237 | 2.230 |
| 11 | 2 | 198651174 | 10497809 | 5495 | TTGGCAAATARTATTCGATTA | 0.892 | 0.891 | 2.530 | 2.343 | 2.628 |
| 11 | 2 | 198671129 | 16826320 | 5496 | TTTGCAGTTAYAATGGAGCTG | 2.505 | 2.450 | 3.024 | 2.977 | 2.794 |
| 11 | 2 | 198678142 | 2139050 | 5497 | CTTATGTTGGRGAAGTTGATT | 3.055 | 2.742 | 3.221 | 3.198 | 2.533 |
| 11 | 2 | 198684689 | 1518369 | 5498 | AAAGTCATCTRAGTCAGAAAT | 2.168 | 1.994 | 3.271 | 3.221 | 2.605 |
| 11 | 2 | 198693707 | 1976772 | 5499 | CTGGACTCTTKTATTGGTTCG | 0.574 | 0.599 | 3.156 | 3.369 | 2.563 |
| 11 | 2 | 198704803 | 1440090 | 5500 | TTTTGTTGTTKATGTTATCTT | 2.221 | 2.000 | 3.071 | 3.071 | 2.388 |
| 11 | 2 | 198707174 | 1016883 | 5501 | CTCAAAGTTARAGACCATGAA | 2.439 | 2.314 | 2.462 | 2.749 | 2.417 |
| 11 | 2 | 198719433 | 16826740 | 5502 | GGCTTTTCTGRTGATTCTTAC | 2.259 | 2.181 | 2.482 | 2.260 | 2.592 |
| 11 | 2 | 198730776 | 892514 | 5503 | TTCTCCTTGGYGGGTGGAATT | 0.006 | 0.009 | 1.647 | 1.746 | 2.607 |
| 11 | 2 | 198736751 | 10497812 | 5504 | CTTTATAATCRAGGGCTAAAT | 0.426 | 0.447 | 1.145 | 1.843 | 2.226 |
| 11 | 2 | 198746066 | 13382697 | 5505 | TAGGGATGAAMTAATCACTGT | 0.137 | 0.141 | 0.576 | 1.308 | 1.866 |
| 11 | 2 | 198755222 | 6434954 | 5506 | GAGGGGCAGCYAAGATTTATG | 1.326 | 1.355 | 0.577 | 0.916 | 1.462 |
| 11 | 2 | 198765757 | 7590828 | 5507 | CTCATAACTGYGTACGAAGCA | 0.689 | 0.706 | 0.519 | 0.348 | 1.729 |
| 11 | 2 | 198773023 | 16827521 | 5508 | TAGGAAGGACRATAGGTGAGA | 0.024 | 0.023 | 0.772 | 0.474 | 1.363 |
| 11 | 2 | 198785109 | 3755333 | 5509 | TGCCTATCTARTATTTTGTGC | 0.286 | 0.290 | 0.238 | 0.389 | 0.792 |
| 11 | 2 | 198791257 | 7570678 | 5510 | TACATTTATTYGACAGCTACT | 0.755 | 0.786 | 0.175 | 0.671 | 0.347 |
| 11 | 2 | 198798289 | — | 5511 | ATCTTTTTCTRTACGATTCTT | 0.057 | 0.058 | 0.211 | 0.460 | 0.417 |
| 11 | 2 | 198808027 | 16827729 | 5512 | AAGCTTCTTARCAATGTATAT | 0.494 | 0.515 | 0.545 | 0.297 | 0.352 |
| 11 | 2 | 198814784 | 16827788 | 5513 | TAGTCCTGGGWAATTGGGACC | 0.134 | 0.135 | 0.501 | 0.321 | 0.521 |
| 11 | 2 | 198821526 | 16827819 | 5514 | TTCATGGACASCTCACCTTCT | 1.115 | 1.064 | 0.526 | 0.353 | 0.264 |
| 11 | 2 | 198834891 | 16827923 | 5515 | GAAAATGGCCMAGATAAAAAT | 0.695 | 0.703 | 0.363 | 0.201 | 0.187 |
| 11 | 2 | 198843361 | 11896429 | 5516 | ATTGAGGCACWACTGAAAAAG | 0.112 | 0.113 | 0.452 | 0.428 | 0.259 |
| 11 | 2 | 198850476 | 13031577 | 5517 | TAAAAGTTATRTTGACTGGCT | 0.124 | 0.124 | 0.143 | 0.316 | 0.232 |
| 11 | 2 | 198863571 | 1368990 | 5518 | ACACTAACTTYTTTGCTCTCA | 0.393 | 0.395 | 0.229 | 0.323 | 0.267 |
| 11 | 2 | 198873644 | 1434293 | 5519 | GTGGCCATGCMTGAGGTTGAG | 0.155 | 0.150 | 0.219 | 0.198 | 0.270 |
| 11 | 2 | 198882620 | 3937566 | 5520 | GTTTGTATGGYCTATTGCATA | 0.991 | 0.984 | 0.243 | 0.101 | 0.264 |
| 11 | 2 | 198899396 | 4850447 | 5521 | CCATTTATAGYGTTCCATGTG | 0.045 | 0.045 | 0.274 | 0.245 | 0.281 |
| 11 | 2 | 198905312 | 1598660 | 5522 | TTCACTCTGAYTAATGGATGG | 0.162 | 0.164 | 0.233 | 0.239 | 0.142 |
| 11 | 2 | 198912596 | 6746027 | 5523 | CAAGGTTTCAYTTGAGGTGCC | 0.516 | 0.504 | 0.237 | 0.256 | 0.080 |
| 11 | 2 | 198918962 | 13411216 | 5524 | ATGACTGGTAYACTTTACACA | 0.132 | 0.133 | 0.239 | 0.267 | 0.078 |
| 11 | 2 | 198924942 | 12693836 | 5525 | AACAGGAGAAYGGAATCCCTG | 1.012 | 0.999 | 0.364 | 0.096 | 0.098 |
| 11 | 2 | 198934154 | 11674810 | 5526 | CTGCTTTTCTRTGGTCACATC | 0.066 | 0.068 | 0.274 | 0.100 | 0.154 |
| 11 | 2 | 198940187 | 6434961 | 5527 | TAAGCAAATTYGGAGATACAG | 0.426 | 0.432 | 0.272 | 0.108 | 0.175 |
| 11 | 2 | 198947953 | 1456556 | 5528 | AAGCCAAGGAYTGGATCATTT | 0.250 | 0.258 | 0.037 | 0.094 | 0.121 |
| 11 | 2 | 198954384 | 16829186 | 5529 | GATATGGTTCRGCTGTGGCGA | 0.068 | 0.068 | 0.051 | 0.239 | 0.124 |
| 11 | 2 | 198964749 | 4850838 | 5530 | AGGAAATCTCRTAATTGAGAA | 0.106 | 0.106 | 0.041 | 0.114 | 0.359 |
| 11 | 2 | 198977567 | 12471228 | 5531 | TGAACTTAATYCTTTATCCAA | 0.170 | 0.171 | 0.188 | 0.193 | 0.289 |
| 11 | 2 | 198987602 | 1037663 | 5532 | CCTCCAAATCSTTCCTGAGAG | 0.357 | 0.369 | 0.281 | 0.125 | 0.346 |
| 11 | 2 | 198995021 | 13398693 | 5533 | CTTAATGTTAYGGTGATTTTT | 0.909 | 0.920 | 0.430 | 0.423 | 0.221 |
| 11 | 2 | 199003237 | 2529670 | 5534 | TACTACTTTCSCTTAACAAGG | 0.353 | 0.357 | 0.375 | 0.446 | 0.367 |
| 11 | 2 | 199009513 | 13022622 | 5535 | CTGACAAATCRAAGCAGAAAA | 0.493 | 0.521 | 0.992 | 0.552 | 0.359 |
| 11 | 2 | 199015891 | 2727767 | 5536 | TCATATGCTTRTTGTGACTTG | 0.069 | 0.070 | 0.586 | 0.595 | 0.372 |
| 11 | 2 | 199018779 | 16829952 | 5537 | GCTTTTTCCARATAGAGGTGT | 1.610 | 1.708 | 0.618 | 0.762 | 0.477 |
| 11 | 2 | 199030492 | 10490082 | 5538 | TCTGTATTATWCTTAATGGTG | 0.145 | 0.148 | 0.527 | 0.579 | 0.468 |
| 11 | 2 | 199042202 | — | 5539 | CTAGGAATCTKGCAAAGTGGA | 0.451 | 0.444 | 0.900 | 0.562 | 0.436 |
| 11 | 2 | 199049683 | 12468958 | 5540 | TGAAAGAGACRGAGTCTAAGA | 0.268 | 0.289 | 0.368 | 0.570 | 0.537 |
| 11 | 2 | 199056937 | 2467041 | 5541 | ATATGTATTCYGGCTAGACTG | 0.885 | 0.922 | 0.455 | 0.578 | 0.381 |
| 11 | 2 | 199063519 | 6744584 | 5542 | TGTAAGTACAYTGGCTTTTAT | 0.362 | 0.361 | 0.496 | 0.180 | 0.330 |
| 11 | 2 | 199071714 | 2880591 | 5543 | CTTTATAGGTRCATACTTATG | 0.287 | 0.304 | 0.387 | 0.341 | 0.360 |
| 11 | 2 | 199078721 | 2041080 | 5544 | GGATATTGGCSTGAAGATGTA | 0.558 | 0.563 | 0.095 | 0.288 | 0.435 |
| 12 | 3 | 6629288 | 6774186 | 5545 | TGAGATGGCASATTTTTAATA | 1.051 | 1.052 | 1.199 | 1.388 | 1.231 |
| 12 | 3 | 6641674 | 2323812 | 5546 | CTAAAGCCCARTGATATATAA | 0.807 | 0.786 | 1.785 | 1.427 | 1.339 |
| 12 | 3 | 6654184 | 17045432 | 5547 | TCCAGCTGGCSAAACTCATGT | 1.147 | 1.141 | 1.678 | 1.203 | 1.579 |
| 12 | 3 | 6661203 | 9843107 | 5548 | GGCTGCTGCARGGAATCAGTT | 1.403 | 1.442 | 1.261 | 1.774 | 1.602 |
| 12 | 3 | 6668609 | 17045515 | 5549 | TTTAGCCTCAYTGGGATTCTG | 0.195 | 0.199 | 1.060 | 1.777 | 2.133 |
| 12 | 3 | 6674962 | 17045532 | 5550 | GTTGTCAGATMCCAAGGTTTT | 0.287 | 0.275 | 1.167 | 1.376 | 2.144 |
| 12 | 3 | 6681752 | 17045568 | 5551 | CCATAGGGACMGGTGCACACT | 0.471 | 0.497 | 0.545 | 1.426 | 2.204 |
| 12 | 3 | 6687829 | 9822640 | 5552 | TAAATCATTCKGGGGCTTCCA | 1.403 | 1.330 | 0.638 | 1.337 | 2.051 |
| 12 | 3 | 6694368 | 17045665 | 5553 | GTTGGTCGCCMAAACACTAAA | 0.279 | 0.270 | 1.034 | 1.306 | 1.590 |
| 12 | 3 | 6700175 | 17045707 | 5554 | GCAAAACATCYTGCAATATTA | 0.389 | 0.387 | 1.270 | 1.518 | 1.373 |
| 12 | 3 | 6715694 | 17045781 | 5555 | ATAAATGTTGYCAAATTTGGA | 1.238 | 1.187 | 1.206 | 1.370 | 0.978 |
| 12 | 3 | 6723760 | 9817687 | 5556 | AAATGCCTTAYGATTTAATCT | 1.001 | 0.980 | 1.439 | 1.175 | 0.560 |
| 12 | 3 | 6732331 | 17460972 | 5557 | TGCCTGAAAARTGGTAGGTAG | 1.202 | 1.234 | 1.269 | 0.733 | 0.709 |
| 12 | 3 | 6745752 | 345233 | 5558 | GGGATTGCTAYTTTAAGAAGT | 0.640 | 0.608 | 0.697 | 0.651 | 0.626 |
| 12 | 3 | 6753268 | 163966 | 5559 | AGTAGGTAACRCCCATTTGTT | 0.176 | 0.177 | 0.399 | 0.523 | 0.513 |
| 12 | 3 | 6759934 | 347078 | 5560 | GACTAATCAASATCAATTCAC | 0.005 | 0.005 | 0.053 | 0.388 | 0.362 |
| 12 | 3 | 6770637 | 345224 | 5561 | GCAGCCATAASTCTCTAAAAG | 0.295 | 0.289 | 0.002 | 0.167 | 0.451 |
| 12 | 3 | 6780770 | 191525 | 5562 | CCAGCTGTATWGGTTTTGCTT | 0.018 | 0.017 | 0.061 | 0.022 | 0.426 |
| 12 | 3 | 6789988 | 6781770 | 5563 | TGTGGTTTGAYTACCTGAACG | 0.004 | 0.004 | 0.062 | 0.026 | 0.207 |
| 12 | 3 | 6798823 | 11714467 | 5564 | ACTTCTTTTGRCAGGTATGTG | 0.779 | 0.765 | 0.023 | 0.070 | 0.239 |
| 12 | 3 | 6807712 | 12635360 | 5565 | ACAGTTGCCAMCTCTCTACCC | 0.024 | 0.024 | 0.168 | 0.112 | 0.098 |
| 12 | 3 | 6825351 | 6776458 | 5566 | ACTCATTTCGKTTGTTTCCTT | 0.036 | 0.036 | 0.375 | 0.076 | 0.067 |
| 12 | 3 | 6834633 | 11929454 | 5567 | ATTGTGCCTCRGCTCATAAAG | 0.739 | 0.707 | 0.223 | 0.299 | 0.058 |
| 12 | 3 | 6842256 | 17046136 | 5568 | CACTTCCTAAYGGGAAACCAC | 0.632 | 0.630 | 0.224 | 0.331 | 0.181 |
| 12 | 3 | 6849819 | 437555 | 5569 | ACACCAGCAGRGCGTCCAATG | 0.297 | 0.291 | 0.690 | 0.215 | 0.420 |
| 12 | 3 | 6858651 | 449379 | 5570 | TACTCTTGCCRGTTTTATACT | 0.042 | 0.041 | 0.433 | 0.225 | 0.415 |
| 12 | 3 | 6868897 | 7651971 | 5571 | CTAGAGACTCYACTAAATGCT | 1.217 | 1.169 | 0.278 | 0.546 | 0.495 |
| 12 | 3 | 6880612 | 340660 | 5572 | ACTAGGCTGASGGACTGTGTT | 0.143 | 0.145 | 0.202 | 0.867 | 0.348 |
| 12 | 3 | 6887801 | 460041 | 5573 | TCTGCAGTAAWACTGAACAAA | 0.289 | 0.285 | 0.678 | 0.595 | 0.546 |
| 12 | 3 | 6899331 | 1240966 | 5574 | TGTTTGTGGGKGTACACACAG | 0.041 | 0.042 | 0.848 | 0.616 | 0.936 |
| 12 | 3 | 6913008 | 9882865 | 5575 | TGAAAGAATTRTGTGTCTTCT | 1.216 | 1.177 | 0.768 | 0.663 | 0.764 |
| 12 | 3 | 6920735 | — | 5576 | TGTATATATCRTCTACGTGTC | 1.532 | 1.535 | 0.792 | 0.533 | 0.635 |
| 12 | 3 | 6928331 | 17046336 | 5577 | CATCTTTGCTRTTTATTTTTC | 0.027 | 0.026 | 0.836 | 0.914 | 0.703 |
| 12 | 3 | 6939637 | 339013 | 5578 | GGCCACTGACRGGAATAAAGT | 0.302 | 0.315 | 0.658 | 0.888 | 0.910 |
| 12 | 3 | 6946081 | 1516561 | 5579 | TGCCCAAAATKAACTCAGAAT | 0.116 | 0.117 | 0.522 | 0.932 | 0.599 |
| 12 | 3 | 6952098 | 17693917 | 5580 | ATATGGCAAARCTCAAAGAAT | 0.855 | 0.848 | 0.631 | 0.728 | 0.555 |
| 12 | 3 | 6956587 | 535178 | 5581 | TTTTCATTTCRTGATTTACAA | 1.247 | 1.238 | 0.599 | 0.469 | 0.501 |
| 12 | 3 | 6969753 | 6443077 | 5582 | TTTTAGTGACYGAACGTGAAG | 0.252 | 0.231 | 0.795 | 0.491 | 0.492 |
| 12 | 3 | 6978781 | 483162 | 5583 | TTTATGGTACRTCCTCATTCA | 0.313 | 0.302 | 0.722 | 0.397 | 0.268 |
| 12 | 3 | 6986240 | 802783 | 5584 | ATAAAGAGCTMTGTCAGGTTC | 0.565 | 0.542 | 0.229 | 0.364 | 0.101 |
| 12 | 3 | 6993932 | 781388 | 5585 | ATGTTATCTTYACCAAAAACG | 0.674 | 0.685 | 0.162 | 0.163 | 0.342 |
| 12 | 3 | 7000186 | 655620 | 5586 | GGTCAGAAAGRGATACATCAA | 0.057 | 0.058 | 0.097 | 0.025 | 0.300 |
| 12 | 3 | 7006247 | 28407476 | 5587 | ATTGTCAATCYTGATCATACA | 0.054 | 0.055 | 0.023 | 0.030 | 0.286 |
| 12 | 3 | 7018599 | 2201924 | 5588 | ATGTGTGTGCWTGGACTCTTA | 0.048 | 0.046 | 0.000 | 0.235 | 0.436 |
| 12 | 3 | 7024917 | 1240472 | 5589 | GAAGGAGTTAYAGGGCTAATT | 0.044 | 0.043 | 0.005 | 0.150 | 0.230 |
| 12 | 3 | 7030300 | 17046535 | 5590 | TATATGAGTTYGTTTGAATTA | 0.065 | 0.065 | 0.355 | 0.061 | 0.203 |
| 12 | 3 | 7036890 | 1154367 | 5591 | TAGGATAATAYGTTAAAAGGT | 0.336 | 0.336 | 0.366 | 0.353 | 0.327 |
| 12 | 3 | 7043889 | 6800531 | 5592 | CCTCTTTTGTRTTGTTAGCTG | 1.668 | 1.609 | 0.357 | 0.402 | 0.277 |
| 12 | 3 | 7055901 | 234790 | 5593 | GAGTTTGCATSGTTCCTTTCA | 0.052 | 0.054 | 0.990 | 0.382 | 0.241 |
| 12 | 3 | 7061757 | 1499147 | 5594 | GTTAATGTGAKGAAGGGTGGT | 0.066 | 0.066 | 0.975 | 0.673 | 0.271 |
| 12 | 3 | 7068995 | 17046736 | 5595 | TAGTTTGTTARTTGAACTCCT | 1.566 | 1.514 | 0.263 | 0.712 | 0.456 |
| 12 | 3 | 7077260 | 6443079 | 5596 | TGAGTCAGGARCATCATTAAT | 0.205 | 0.209 | 0.664 | 0.779 | 0.961 |
| 12 | 3 | 7084915 | 1499156 | 5597 | CAGTGGAACARCTGAGCAAAG | 0.011 | 0.011 | 0.737 | 0.349 | 1.055 |
| 12 | 3 | 7095417 | — | 5598 | TTTAACTTGTRAGGTGTAGCC | 1.025 | 1.046 | 0.289 | 0.649 | 1.159 |
| 12 | 3 | 7102850 | 7625532 | 5599 | ATTCTAGTTAYGTTCACTGGA | 0.242 | 0.243 | 0.298 | 1.367 | 1.793 |
| 12 | 3 | 7114845 | 6769115 | 5600 | TTCTGTCTTCYGATCATTCAA | 0.000 | 0.000 | 0.799 | 0.967 | 3.559 |
| 12 | 3 | 7122158 | 17046783 | 5601 | TCATGAATTTYGGCATTTTTT | 0.269 | 0.270 | 1.489 | 1.084 | 3.441 |
| 12 | 3 | 7128652 | 6778030 | 5602 | TTGACTATCTRTGAAAACTGT | 1.010 | 1.026 | 1.363 | 2.097 | 3.415 |
| 12 | 3 | 7136316 | 17234935 | 5603 | TCATAATCTASGACTGGATAT | 2.104 | 2.063 | 1.272 | 4.120 | 4.568 |
| 12 | 3 | 7142442 | 11915246 | 5604 | TATTAGAGACYTGTTGGCAGT | 0.405 | 0.400 | 2.306 | 3.966 | 4.568 |
| 12 | 3 | 7148002 | 1878164 | 5605 | ATGACACTTCRTCTACTTGAA | 0.584 | 0.576 | 4.488 | 3.643 | 4.789 |
| 12 | 3 | 7155162 | 6804466 | 5606 | ACTTTATAGGYATACTGGTAG | 2.722 | 2.430 | 4.120 | 4.153 | 4.364 |
| 12 | 3 | 7162872 | 17234969 | 5607 | ACAACCATGAYCCTGACCTTG | 6.278 | 4.966 | 3.886 | 4.789 | 4.187 |
| 12 | 3 | 7168821 | 7623514 | 5608 | TCCTCAAGAAMGACTTGCATT | 0.651 | 0.690 | 4.312 | 4.966 | 4.225 |
| 12 | 3 | 7174495 | 6443093 | 5609 | AATGTAGAAGYGGCAGAAGAC | 0.979 | 0.966 | 4.789 | 5.267 | 4.091 |
| 12 | 3 | 7187512 | 17235018 | 5610 | AAACCTACATSATTCTGTATG | 4.807 | 4.789 | 3.454 | 4.966 | 4.091 |
| 12 | 3 | 7193175 | 1400166 | 5611 | TCATCCTTTCRTCTTTATTCA | 0.785 | 0.786 | 3.093 | 5.267 | 3.886 |
| 12 | 3 | 7198739 | 13082571 | 5612 | GGCTAAAGAAYAGTACAAACC | 0.735 | 0.742 | 2.601 | 1.870 | 4.062 |
| 12 | 3 | 7204619 | 11708019 | 5613 | CAGTTGTCTCRATGCCTAGTA | 0.120 | 0.121 | 0.354 | 1.694 | 3.924 |
| 12 | 3 | 7211588 | 11918486 | 5614 | TTGCTCAGGGWTTCTATGATA | 0.378 | 0.363 | 0.122 | 1.687 | 3.665 |
| 12 | 3 | 7220908 | 2133440 | 5615 | GACAGAAAGAMAATGGTTGTG | 0.111 | 0.107 | 0.020 | 0.521 | 1.766 |
| 12 | 3 | 7227190 | 10510354 | 5616 | TGTTACAGAASGTATGTGTTT | 0.028 | 0.028 | 0.213 | 0.346 | 1.711 |
| 12 | 3 | 7233031 | 17638908 | 5617 | TGATAATTGASTAAAGGCAAG | 0.083 | 0.078 | 0.504 | 0.252 | 1.455 |
| 12 | 3 | 7239504 | 6414460 | 5618 | TTCAAGTGTTWGTTTGGAATA | 1.117 | 1.030 | 0.503 | 0.430 | 0.371 |
| 12 | 3 | 7251549 | 1508713 | 5619 | TATTTAATATYCTTGAAAGGT | 0.929 | 0.856 | 0.629 | 0.420 | 0.285 |
| 12 | 3 | 7259458 | 870440 | 5620 | CTCTGATCTAYTCCAGAGCTG | 0.137 | 0.135 | 1.064 | 0.406 | 0.231 |
| 12 | 3 | 7266260 | 2136153 | 5621 | GTCTCTGATAMGGGAAACAAA | 0.353 | 0.340 | 0.684 | 0.511 | 0.267 |
| 12 | 3 | 7274331 | 9873905 | 5622 | AGAAACCTAGYGGTAGCAAGT | 0.870 | 0.845 | 0.227 | 0.589 | 0.238 |
| 12 | 3 | 7286253 | 9829398 | 5623 | TGAAGGGCCAYGTACTACCTC | 0.339 | 0.330 | 0.334 | 0.392 | 0.286 |
| 12 | 3 | 7295514 | 6443099 | 5624 | CTTCACTGAAYGGATAGATAC | 0.068 | 0.069 | 0.331 | 0.207 | 0.316 |
| 12 | 3 | 7305179 | 7609608 | 5625 | CTGGTCCTGCRTTTGGCTCTG | 0.388 | 0.389 | 0.179 | 0.214 | 0.369 |
| 12 | 3 | 7318584 | 17047063 | 5626 | GTTAACTTCARATACTTTGTC | 0.331 | 0.325 | 0.176 | 0.230 | 0.243 |
| 12 | 3 | 7329377 | 9866393 | 5627 | AGGATGAACARGTGACTTAAT | 0.393 | 0.370 | 0.176 | 0.107 | 0.302 |
| 12 | 3 | 7336512 | 2030154 | 5628 | TTGTTGCCTTWGATTATTATT | 0.382 | 0.386 | 0.168 | 0.106 | 0.297 |
| 12 | 3 | 7344342 | 9311986 | 5629 | GCAGTTGGATSACTTGATTTT | 0.087 | 0.087 | 0.136 | 0.144 | 0.318 |
| 12 | 3 | 7357930 | 17047171 | 5630 | CTTTCTAGAGWTTACATGAGG | 0.413 | 0.401 | 0.144 | 0.408 | 0.217 |
| 12 | 3 | 7367719 | 17047199 | 5631 | TTTGTCGAGGWTGCTAAAACC | 0.165 | 0.165 | 0.143 | 0.345 | 0.174 |
| 12 | 3 | 7379067 | 2221668 | 5632 | AAATTCAAAAYGAGAGAAAAT | 0.368 | 0.363 | 0.685 | 0.370 | 0.228 |
| 12 | 3 | 7386549 | 1548146 | 5633 | AAATAAGATTYTTTTGAATAG | 0.370 | 0.378 | 0.553 | 0.331 | 0.214 |
| 12 | 3 | 7394156 | 9883258 | 5634 | ATCAATAGTGRGTGCTTACGG | 1.589 | 1.755 | 0.688 | 0.335 | 0.416 |
| 12 | 3 | 7400433 | 4686127 | 5635 | GCATTCAAGTMATTCAAATAT | 0.105 | 0.110 | 0.630 | 0.347 | 0.379 |
| 12 | 3 | 7408728 | 1499079 | 5636 | GCTGGTGCAGKTTCACGTTTT | 0.458 | 0.459 | 0.516 | 0.388 | 0.531 |
| 12 | 3 | 7415256 | — | 5637 | TTTAAAGGCARTTGGAGAAAA | 0.219 | 0.224 | 0.151 | 0.696 | 0.640 |
| 12 | 3 | 7420630 | 1066658 | 5638 | GATATATGAGYGATGAAATTG | 0.106 | 0.109 | 0.186 | 0.631 | 0.705 |
| 12 | 3 | 7426750 | 17700470 | 5639 | TGACAACCACRTACAGCTCAA | 0.474 | 0.478 | 0.519 | 0.499 | 0.774 |
| 12 | 3 | 7433710 | 712775 | 5640 | CCTCTGACTTYTATGAGCTTA | 0.253 | 0.263 | 0.505 | 0.620 | 0.799 |
| 12 | 3 | 7441454 | 17047321 | 5641 | ATTCCAGTACRGGAGGCAGAA | 1.440 | 1.485 | 0.924 | 0.688 | 1.117 |
| 12 | 3 | 7448956 | 11717280 | 5642 | TAGGAGTGAAYGCCAGTGTAA | 0.160 | 0.163 | 0.993 | 0.767 | 0.746 |
| 12 | 3 | 7461021 | 712786 | 5643 | TCTATGGCTTRTAGAAGAGGA | 1.079 | 1.097 | 1.287 | 0.910 | 0.716 |
| 12 | 3 | 7467488 | — | 5644 | TGGTATTTAARTGAGATACAT | 0.576 | 0.553 | 0.714 | 1.397 | 0.694 |
| 12 | 3 | 7473748 | 713291 | 5645 | TTTCGAATCCRTTGTGAAAAA | 0.701 | 0.657 | 0.875 | 1.430 | 0.737 |
| 12 | 3 | 7481105 | 1450092 | 5646 | AGCTCAGACARCAGTTGTGAA | 0.490 | 0.482 | 1.210 | 0.780 | 0.751 |
| 12 | 3 | 7487728 | 779708 | 5647 | TTCCTGAACCRCAATTCCCTT | 0.413 | 0.406 | 0.929 | 0.852 | 0.959 |
| 12 | 3 | 7499103 | 779705 | 5648 | AAAACTGATAYGTAGGTAACC | 1.720 | 1.853 | 0.584 | 0.591 | 1.097 |
| 12 | 3 | 7508618 | 10222587 | 5649 | CTGTTCTACCYGGCAAGGACC | 0.137 | 0.132 | 0.493 | 0.438 | 0.796 |
| 12 | 3 | 7517268 | 1375923 | 5650 | TGATATTGGAYGATACTGATT | 0.004 | 0.004 | 0.502 | 0.596 | 0.893 |
| 12 | 3 | 7526367 | 924354 | 5651 | CAGTAACAATKTTTCTTTAGT | 0.298 | 0.294 | 0.041 | 0.654 | 0.675 |
| 12 | 3 | 7532504 | 1145137 | 5652 | TAAACTAGGGRACAATGACTT | 0.396 | 0.392 | 0.307 | 0.673 | 0.704 |
| 12 | 3 | 7540619 | 1144026 | 5653 | TAGTTTTCTGSGTTTTGTTGT | 0.143 | 0.135 | 0.598 | 0.344 | 0.657 |
| 12 | 3 | 7552841 | 17721161 | 5654 | TGCTGGGACTYGGTTTCTGAA | 1.200 | 1.222 | 0.667 | 0.379 | 0.553 |
| 12 | 3 | 7562251 | 779736 | 5655 | TTTTAACCAGRAAGTCTTTGT | 0.699 | 0.664 | 0.728 | 0.574 | 0.466 |
| 12 | 3 | 7571446 | 779719 | 5656 | TCCCTTCTCTRTAATCAGCAG | 0.523 | 0.511 | 0.790 | 0.631 | 0.324 |
| 12 | 3 | 7578708 | 1485179 | 5657 | GAGTTTGAAAYTCCAACTGCT | 0.501 | 0.513 | 0.553 | 0.536 | 0.467 |
| 12 | 3 | 7579955 | 1145141 | 5658 | AGGCTCAAGCRCAAAAGAAGA | 0.228 | 0.234 | 0.472 | 0.502 | 0.482 |
| 12 | 3 | 7592025 | 1531939 | 5659 | TTTTCTTAATSTTTCTGCACC | 0.731 | 0.680 | 0.301 | 0.455 | 0.656 |
| 12 | 3 | 7604004 | 4686140 | 5660 | AATTAGAGTAWGTAAAGCTCT | 0.000 | 0.000 | 0.142 | 0.488 | 0.682 |
| 12 | 3 | 7614708 | 3804883 | 5661 | TCAGGAAGACRAAAGGACCAA | 0.121 | 0.120 | 0.440 | 0.358 | 0.672 |
| 12 | 3 | 7620217 | 11713266 | 5662 | TTGTGCAAGCKAGTTAAATTT | 0.004 | 0.004 | 0.471 | 0.506 | 0.394 |
| 12 | 3 | 7629492 | 1485170 | 5663 | AGAATTCTTGRAAACAGCTTC | 1.036 | 1.057 | 0.312 | 0.579 | 0.319 |
| 12 | 3 | 7635133 | 7640685 | 5664 | AATGCCAAAAYGAACATTTAG | 0.785 | 0.786 | 0.687 | 0.397 | 0.320 |
| 12 | 3 | 7643760 | 3804866 | 5665 | GCTTTGGGTCWCAGCTTCCAC | 0.102 | 0.099 | 0.910 | 0.304 | 0.285 |
| 12 | 3 | 7650322 | 9832222 | 5666 | TACTCCTGCASCTTCTAATTC | 1.019 | 1.011 | 0.478 | 0.368 | 0.320 |
| 12 | 3 | 7656966 | 17724199 | 5667 | AACTACATGCYACTGGGACAG | 0.460 | 0.469 | 0.216 | 0.501 | 0.298 |
| 12 | 3 | 7663564 | 4686146 | 5668 | TTTCCTCAGGKAGCCATGTAA | 0.109 | 0.109 | 0.297 | 0.297 | 0.319 |
| 12 | 3 | 7680399 | — | 5669 | GTTATGGAGASCATGGATTTT | 0.116 | 0.109 | 0.153 | 0.213 | 0.328 |
| 12 | 3 | 7685841 | 2324209 | 5670 | GTCTAGCAAGSGGACACGTAT | 0.327 | 0.324 | 0.113 | 0.334 | 0.414 |
| 12 | 3 | 7691409 | 9845583 | 5671 | TGCAACTAGAMTGGAGCTTTC | 0.513 | 0.513 | 0.191 | 0.233 | 0.251 |
| 12 | 3 | 7698179 | 10514663 | 5672 | AGTGAATGTCRTGAAATACTT | 0.317 | 0.318 | 0.333 | 0.160 | 0.204 |
| 12 | 3 | 7706991 | 1354405 | 5673 | AATGAGTAGCRCACAATACTT | 0.394 | 0.387 | 0.417 | 0.228 | 0.254 |
| 12 | 3 | 7713566 | 162773 | 5674 | ATTTATATATYGGGAGAGCTT | 0.521 | 0.513 | 0.282 | 0.229 | 0.146 |
| 12 | 3 | 7720781 | 13099271 | 5675 | ATATAAAACAWGATAACATTG | 0.509 | 0.532 | 0.358 | 0.271 | 0.106 |
| 12 | 3 | 7733107 | 1857697 | 5676 | AGTTTGTAAAYTGAACATTAT | 0.128 | 0.124 | 0.255 | 0.246 | 0.111 |
| 12 | 3 | 7744798 | 162723 | 5677 | TTGACTGTGAYGGCACACACG | 0.479 | 0.479 | 0.260 | 0.221 | 0.115 |
| 12 | 3 | 7755175 | 17047887 | 5678 | TAACCTCATCRTGATTTTAAC | 0.152 | 0.156 | 0.193 | 0.164 | 0.099 |
| 12 | 3 | 7761554 | 17047896 | 5679 | CTTGTTCCAGRATGCTAGCTT | 0.421 | 0.428 | 0.205 | 0.126 | 0.110 |
| 12 | 3 | 7772492 | 161885 | 5680 | GTCCTTCGAARTCAAAACAGA | 0.334 | 0.325 | 0.089 | 0.067 | 0.222 |
| 12 | 3 | 7780780 | 9817941 | 5681 | ATGTAGCACAYAGGGTGATAT | 0.221 | 0.214 | 0.109 | 0.060 | 0.198 |
| 12 | 3 | 7786740 | 1473278 | 5682 | ATTTTTTTAGSTTCTAGACTC | 0.088 | 0.089 | 0.042 | 0.057 | 0.410 |
| 12 | 3 | 7792722 | 9849633 | 5683 | CTATTAGAGGRTAAGCAGACA | 0.160 | 0.151 | 0.017 | 0.233 | 0.520 |
| 12 | 3 | 7798160 | 1550277 | 5684 | AAGGCTGTTGYTAAGCTGTCC | 0.166 | 0.166 | 0.064 | 0.185 | 0.611 |
| 12 | 3 | 7805201 | — | 5685 | CATATTAGTCYAGTTTTACCA | 0.124 | 0.121 | 0.421 | 0.586 | 0.574 |
| 12 | 3 | 7812783 | 11708939 | 5686 | AGTTTGTGGCMTTTTACACTG | 0.581 | 0.596 | 0.469 | 0.929 | 0.617 |
| 12 | 3 | 7818918 | 1354400 | 5687 | AGTTTGGCATMCTTTGGCATC | 1.244 | 1.272 | 1.293 | 1.207 | 0.587 |
| 12 | 3 | 7825284 | 6780542 | 5688 | GACAATTATAYGCATTGTTTC | 0.290 | 0.295 | 1.827 | 1.215 | 0.625 |
| 12 | 3 | 7831694 | 17048003 | 5689 | TGAATGGTACYAAGAAAGAAT | 1.817 | 1.908 | 1.973 | 1.265 | 0.660 |
| 12 | 3 | 7838157 | 162707 | 5690 | GAAGGGTTTARAATCCTTGTT | 1.120 | 1.094 | 1.233 | 1.227 | 0.882 |
| 12 | 3 | 7850799 | 12488799 | 5691 | GCATAAACTCRGGAAACGACA | 0.525 | 0.523 | 1.210 | 1.094 | 0.822 |
| 12 | 3 | 7859798 | 17048056 | 5692 | TTGTGTTTTGKATCAGAGCTG | 0.335 | 0.326 | 0.542 | 0.819 | 0.875 |
| 12 | 3 | 7871679 | 11718722 | 5693 | AACCCTGGAGYCAGATTTGTC | 0.351 | 0.343 | 0.343 | 0.992 | 1.002 |
| 12 | 3 | 7877712 | 17740513 | 5694 | ACAGGGCAAGRATGGTGTGAT | 0.303 | 0.308 | 0.286 | 0.523 | 0.864 |
| 12 | 3 | 7884368 | 9813540 | 5695 | TTTTTGAGGTYGAAGCTTTAT | 0.524 | 0.501 | 0.469 | 0.336 | 0.621 |
| 12 | 3 | 7890962 | 401263 | 5696 | GACATGAAAAYGGAATGAGAT | 0.362 | 0.351 | 0.401 | 0.354 | 0.572 |
| 12 | 3 | 7900483 | 378055 | 5697 | GCGATTAAAARTAGATATTCT | 0.930 | 0.889 | 0.388 | 0.324 | 0.323 |
| 12 | 3 | 7909333 | 1001254 | 5698 | GAATGTTTTASCTAGCAGCCT | 0.149 | 0.146 | 0.416 | 0.282 | 0.170 |
| 12 | 3 | 7924234 | — | 5699 | AGCGTTGATTKGCACTAGAGT | 0.317 | 0.325 | 0.359 | 0.239 | 0.117 |
| 12 | 3 | 7929783 | 17048336 | 5700 | CCCTTACATAWCATAATAGCA | 0.602 | 0.618 | 0.145 | 0.169 | 0.122 |
| 12 | 3 | 7937060 | 7625080 | 5701 | CACTCCAAATRCCCATTCCTG | 0.167 | 0.165 | 0.118 | 0.128 | 0.109 |
| 12 | 3 | 7942629 | 17048368 | 5702 | AGACCTTTGARAAAGAAGTTG | 0.157 | 0.156 | 0.111 | 0.026 | 0.074 |
| 12 | 3 | 7950061 | 10510379 | 5703 | TTATATAGTARTTGTCACGCA | 0.058 | 0.057 | 0.020 | 0.037 | 0.038 |
| 12 | 3 | 7957884 | 7652192 | 5704 | ATTTGACAGTKTATTTACCAG | 0.208 | 0.203 | 0.006 | 0.038 | 0.027 |
| 12 | 3 | 7967737 | 7428715 | 5705 | AGACTCTAATRTTATTTGGTT | 0.120 | 0.119 | 0.021 | 0.006 | 0.007 |
| 12 | 3 | 7980225 | 17693661 | 5706 | TGTCAGTCCTRTATTTTCTGT | 0.035 | 0.034 | 0.043 | 0.004 | 0.005 |
| 12 | 3 | 7987346 | 4459890 | 5707 | GGTAATATGGYTTTTGAATAT | 0.390 | 0.373 | 0.026 | 0.002 | 0.006 |
| 12 | 3 | 7994240 | 17048460 | 5708 | AAACTTTTCARTAAACCATGT | 0.000 | 0.000 | 0.034 | 0.007 | 0.001 |
| 12 | 3 | 8005030 | 17048480 | 5709 | ACTCTCTGTTKCTAAACTAGG | 0.105 | 0.104 | 0.038 | 0.006 | 0.005 |
| 13 | 3 | 32775326 | 6785966 | 5710 | GTAACTTAAAYGTTGTCTTCA | 0.098 | 0.100 | 0.262 | 0.195 | 0.110 |
| 13 | 3 | 32780201 | 6550159 | 5711 | TTACTCAAGAYTAGTGCTCAG | 1.185 | 1.197 | 0.453 | 0.127 | 0.060 |
| 13 | 3 | 32801772 | 7619274 | 5712 | CAGCCTGGGCRACACACAGCG | 0.119 | 0.120 | 0.406 | 0.128 | 0.024 |
| 13 | 3 | 32814515 | 6774262 | 5713 | TGTCTATCATMTATTGTTATA | 0.578 | 0.577 | 0.373 | 0.115 | 0.019 |
| 13 | 3 | 32851743 | 7644738 | 5714 | ATGGGGGGAGYGGGGGCAGAA | 0.243 | 0.320 | 0.067 | 0.076 | 0.019 |
| 13 | 3 | 32865318 | 6550166 | 5715 | GGTCACAGGAMAACCAGAAAG | 0.045 | 0.045 | 0.057 | 0.082 | 0.029 |
| 13 | 3 | 32879421 | 6550167 | 5716 | TTGCGAGATAKAACTTAAGAT | 0.077 | 0.078 | 0.009 | 0.008 | 0.067 |
| 13 | 3 | 32890863 | 7612206 | 5717 | CTACTACTACWACAACATATT | 0.039 | 0.039 | 0.002 | 0.011 | 0.057 |
| 13 | 3 | 32917788 | 1599831 | 5718 | GTCTGGAGTASGTAATGCCAA | 0.173 | 0.178 | 0.003 | 0.005 | 0.114 |
| 13 | 3 | 32930887 | 7643959 | 5719 | GGTATTTGAASAGCCACTCCT | 0.066 | 0.067 | 0.008 | 0.018 | 0.040 |
| 13 | 3 | 32942855 | 6786990 | 5720 | TTTTCTTTGTKAAGCTCTTCC | 0.109 | 0.109 | 0.058 | 0.036 | 0.037 |
| 13 | 3 | 32954092 | 6550177 | 5721 | GGGAGAGGGAYGAGGGGAGGA | 0.000 | 0.000 | 0.165 | 0.109 | 0.283 |
| 13 | 3 | 32970932 | 2228428 | 5722 | TCCAAATTTAYTCTGCTGACA | 0.388 | 0.382 | 0.300 | 0.189 | 0.452 |
| 13 | 3 | 32977803 | 6803961 | 5723 | CCAAAGCAGCWGAGATCAGGT | 0.660 | 0.677 | 0.587 | 0.166 | 1.384 |
| 13 | 3 | 32995794 | 4640506 | 5724 | TTCCTAATAARGAAAGATTTC | 0.000 | 0.000 | 0.587 | 1.039 | 1.309 |
| 13 | 3 | 33009602 | 4075736 | 5725 | TTTCATAAAGRAGAGAAATAA | 0.621 | 0.641 | 0.456 | 1.414 | 1.316 |
| 13 | 3 | 33019646 | 4074708 | 5726 | ATGGTTTCTCYATATTTTGGG | 0.000 | 0.000 | 1.434 | 2.229 | 2.381 |
| 13 | 3 | 33036155 | 6550190 | 5727 | TCCCGCCCAGRCTCCACATCT | 0.103 | 0.103 | 1.566 | 2.054 | 3.159 |
| 13 | 3 | 33046631 | 6762132 | 5728 | CAGGAGGTATRAACAGTGCTG | 2.293 | 2.228 | 2.616 | 2.048 | 3.604 |
| 13 | 3 | 33062204 | 6780220 | 5729 | CTGCCTTAATMGGGGCTATAG | 0.995 | 1.027 | 2.280 | 3.127 | 3.397 |
| 13 | 3 | 33074192 | 4438612 | 5730 | GTGTCCTGCCRAGGTGGGAGG | 3.074 | 2.951 | 2.595 | 3.852 | 3.332 |
| 13 | 3 | 33085660 | 7610916 | 5731 | TCACGGTGGGYGGATGGCTTG | 0.034 | 0.040 | 3.447 | 3.852 | 3.275 |
| 13 | 3 | 33121193 | 4578976 | 5732 | TCCAGAGATAYGAGTTGGGAC | 0.280 | 0.282 | 3.551 | 4.062 | 3.217 |
| 13 | 3 | 33133916 | 4678686 | 5733 | TGCAGCCTCARGTGCATCATA | 3.132 | 3.518 | 2.665 | 3.460 | 3.217 |
| 13 | 3 | 33151828 | 7652193 | 5734 | ACTGTGGTTTRCAAAGTATAG | 2.210 | 2.073 | 2.568 | 2.756 | 3.332 |
| 13 | 3 | 33179004 | 4678490 | 5735 | TTCTTTGAACWGACGAGTAAG | 0.000 | 0.000 | 2.807 | 1.901 | 3.447 |
| 13 | 3 | 33191738 | 4465894 | 5736 | CTGGCAGAATRCCTGGAATAG | 0.868 | 0.836 | 1.488 | 2.027 | 2.876 |
| 13 | 3 | 33204889 | 7636399 | 5737 | TCCCACTCACYCCACAACACA | 0.000 | 0.000 | 0.557 | 2.443 | 2.775 |
| 13 | 3 | 33217501 | 4678764 | 5738 | GGAACCAGAAYTAAAAAATTT | 0.620 | 0.601 | 0.380 | 1.261 | 2.269 |
| 13 | 3 | 33223374 | 4535176 | 5739 | TCTTTAGCGCYGGCACTGAGG | 0.169 | 0.175 | 0.619 | 0.590 | 2.349 |
| 13 | 3 | 33243630 | 6798379 | 5740 | ACAAAATTTGRTGGTAAAAGT | 0.162 | 0.147 | 0.449 | 0.918 | 2.446 |
| 13 | 3 | 33256297 | 6800399 | 5741 | TACTTTGGACRGAATAGAAAA | 1.347 | 1.379 | 0.421 | 1.192 | 1.491 |
| 13 | 3 | 33267546 | 4678512 | 5742 | TTCGTGGTAAYAGAGGAGAAT | 0.113 | 0.116 | 0.938 | 1.093 | 0.992 |
| 13 | 3 | 33273594 | 6798170 | 5743 | CAGAGAGCCARCTAGTACTGC | 0.531 | 0.529 | 1.523 | 1.076 | 0.944 |
| 13 | 3 | 33289743 | 4678832 | 5744 | CAGTAGAGGGRCCAATTCTTG | 1.423 | 1.447 | 1.086 | 1.037 | 0.753 |
| 13 | 3 | 33318826 | 6807330 | 5745 | ATGTGTGGTGKTGTGACACCT | 1.551 | 1.558 | 1.389 | 1.121 | 0.700 |
| 13 | 3 | 33348207 | 17029976 | 5746 | CCACACCTCAMTAATCGGATG | 0.308 | 0.309 | 1.149 | 0.817 | 1.002 |
| 13 | 3 | 33350489 | — | 5747 | AGTGTACTTTYAGCTGTGACC | 0.512 | 0.528 | 0.748 | 0.795 | 1.011 |
| 14 | 3 | 39582260 | 1620274 | 5748 | TGGAAACAAGYTCTGAAGTAT | 0.565 | 0.553 | 0.266 | 0.407 | 1.009 |
| 14 | 3 | 39586705 | 816502 | 5749 | CAGCTCTAGGYAATCTGGCCT | 0.278 | 0.281 | 0.156 | 0.616 | 0.979 |
| 14 | 3 | 39608066 | 7640123 | 5750 | CCTCAAATGAYGTGAGTAGAG | 0.419 | 0.411 | 0.199 | 0.951 | 1.085 |
| 14 | 3 | 39617791 | 9863522 | 5751 | TGCCAGCACCRATGCTTTCAA | 0.231 | 0.227 | 0.898 | 1.287 | 1.122 |
| 14 | 3 | 39628600 | 4676652 | 5752 | CTGTTTCTTTSTATTGCTATT | 0.142 | 0.146 | 1.373 | 1.177 | 0.766 |
| 14 | 3 | 39650458 | 4676655 | 5753 | GTGATTGATAYGAAGGAGCAT | 2.312 | 2.252 | 1.823 | 1.160 | 0.835 |
| 14 | 3 | 39663717 | 4586749 | 5754 | TTGTTTTGGARGTCCTAATTA | 1.213 | 1.162 | 1.677 | 1.169 | 0.687 |
| 14 | 3 | 39669773 | 1405796 | 5755 | CAAGTAGGGAYGTGGGTTTTC | 1.397 | 1.468 | 2.082 | 1.040 | 0.899 |
| 14 | 3 | 39680713 | 10510704 | 5756 | CAGTGCATTAMGAGGACAAGA | 0.050 | 0.050 | 0.900 | 1.184 | 0.792 |
| 14 | 3 | 39700502 | 17759069 | 5757 | TTTGGAATATYGATTACACTG | 0.501 | 0.505 | 0.422 | 1.108 | 0.969 |
| 14 | 3 | 39742324 | 17039611 | 5758 | AGTTCTGGACYGCCATATACT | 0.200 | 0.198 | 0.155 | 0.735 | 0.888 |
| 14 | 3 | 39751229 | 17076570 | 5759 | GAATGACTCCYGTGAATGACC | 0.180 | 0.179 | 0.191 | 0.423 | 1.074 |
| 14 | 3 | 39764195 | 1405788 | 5760 | AGTGTAACATYTACATCAATG | 0.568 | 0.551 | 0.373 | 0.350 | 1.054 |
| 14 | 3 | 39774785 | 1918028 | 5761 | TTTCCTTAACRTTTTGCAGTC | 0.029 | 0.029 | 0.324 | 0.347 | 0.545 |
| 14 | 3 | 39784586 | 4676512 | 5762 | TGTGTTGGCCRGCCTATATTG | 1.155 | 1.129 | 0.664 | 0.410 | 0.349 |
| 14 | 3 | 39794224 | 11717218 | 5763 | CACCTGTTAGYGCACCAGAAA | 0.084 | 0.084 | 0.431 | 0.371 | 0.219 |
| 14 | 3 | 39800003 | 11717991 | 5764 | ATAGAATTACRGCTGGGAACT | 1.063 | 1.094 | 0.791 | 0.373 | 0.399 |
| 14 | 3 | 39807162 | 7621402 | 5765 | ATTGAATAATMTCTGTGAGCA | 0.048 | 0.047 | 0.322 | 0.287 | 0.373 |
| 14 | 3 | 39817037 | 17030291 | 5766 | TACAGATTCAMCTTTCTAGTA | 0.827 | 0.804 | 0.362 | 0.427 | 0.788 |
| 14 | 3 | 39823345 | 1918029 | 5767 | ACAATGAACAYCATTTAACAT | 0.018 | 0.018 | 0.105 | 0.416 | 0.836 |
| 14 | 3 | 39837056 | 9867310 | 5768 | ACATGAATACRGTTTTGTTGC | 0.180 | 0.187 | 0.256 | 0.510 | 0.814 |
| 14 | 3 | 39848488 | 1918025 | 5769 | CACCTGGCACYCTGTTGGTCA | 0.215 | 0.212 | 0.346 | 0.838 | 0.842 |
| 14 | 3 | 39868177 | 28539759 | 5770 | GTTTTCAGCTYCTAGCCCTGT | 0.545 | 0.578 | 0.487 | 0.923 | 0.614 |
| 14 | 3 | 39884611 | 7612386 | 5771 | CCATATACTTYTGGGCCCTGC | 1.131 | 1.136 | 1.585 | 0.838 | 0.616 |
| 14 | 3 | 39895005 | 4676531 | 5772 | CAACAGTATGYGCCTGGTACT | 0.345 | 0.350 | 1.636 | 0.892 | 0.453 |
| 14 | 3 | 39898848 | — | 5773 | AGGCTTGTACRTTGGGCAACT | 2.077 | 2.079 | 1.583 | 0.916 | 0.561 |
| 14 | 3 | 39922940 | 7618607 | 5774 | GTGTTCCTCCRTTTAGGAGAG | 0.239 | 0.246 | 0.978 | 0.886 | 0.417 |
| 14 | 3 | 39930070 | 1599903 | 5775 | ATAAACTAGAMCCCCTGAAAT | 0.000 | 0.000 | 0.901 | 0.768 | 0.504 |
| 14 | 3 | 39936970 | 13060371 | 5776 | TCTTAGCTGCYGAGACATTTT | 0.206 | 0.210 | 0.048 | 0.530 | 0.476 |
| 14 | 3 | 39949399 | 6803701 | 5777 | TTGGCACACCSTCTGAATTAG | 0.227 | 0.218 | 0.067 | 0.484 | 0.447 |
| 14 | 3 | 39962741 | 725296 | 5778 | TACCAGAGACYCACCTACATA | 0.051 | 0.052 | 0.101 | 0.074 | 0.366 |
| 14 | 3 | 39967543 | — | 5779 | ACTGAGAAACRGTGGCCATAT | 0.383 | 0.362 | 0.100 | 0.052 | 0.161 |
| 14 | 3 | 39978902 | 17183401 | 5780 | AATTTGGAGAYGGATAAGGAC | 0.477 | 0.480 | 0.135 | 0.034 | 0.149 |
| 14 | 3 | 40028112 | 4309670 | 5781 | TTAAGCTCACRCAAAGCACTA | 0.223 | 0.219 | 0.147 | 0.032 | 0.206 |
| 14 | 3 | 40037923 | 7637285 | 5782 | AGGAGTTCACWTACAATGCAT | 0.410 | 0.411 | 0.090 | 0.021 | 0.294 |
| 14 | 3 | 40051924 | 7630287 | 5783 | AAGGTGCACTKACATAAAAAG | 0.089 | 0.086 | 0.037 | 0.033 | 0.263 |
| 14 | 3 | 40069799 | 11920120 | 5784 | GTTAAGATACYGCCCTGTCTA | 0.088 | 0.090 | 0.021 | 0.476 | 0.830 |
| 14 | 3 | 40076928 | 2887954 | 5785 | CTTATTTCAARGGAAAAACAC | 0.147 | 0.151 | 0.012 | 0.549 | 0.792 |
| 14 | 3 | 40082408 | 9816042 | 5786 | AGCCTGGACTRTGTTTCTCAG | 0.064 | 0.072 | 0.826 | 0.556 | 1.465 |
| 14 | 3 | 40088394 | 17184770 | 5787 | AGGGTCACGARGGAGCTGGCC | 0.325 | 0.323 | 1.182 | 1.309 | 1.413 |
| 14 | 3 | 40095453 | 6790823 | 5788 | GACCCTCTTCSTTCTCCAGTT | 2.441 | 2.719 | 1.130 | 1.286 | 1.387 |
| 14 | 3 | 40105656 | 17075569 | 5789 | AGGACTCCAGYTTTACTGCTA | 0.715 | 0.698 | 2.318 | 1.976 | 1.560 |
| 14 | 3 | 40116861 | 11720413 | 5790 | TGATGTTTTGSAACAGAACTA | 0.253 | 0.259 | 2.020 | 2.073 | 1.626 |
| 14 | 3 | 40124462 | 1799414 | 5791 | CAACTGCATCMGTGTCCCACA | 2.459 | 2.389 | 1.850 | 2.126 | 1.695 |
| 14 | 3 | 40132773 | 1616490 | 5792 | CCTCCATGAGWTGTGTAGACC | 0.053 | 0.054 | 1.727 | 2.433 | 1.812 |
| 14 | 3 | 40142195 | 882822 | 5793 | TGTAGGGATCRGAGAAATTGC | 2.142 | 2.074 | 1.829 | 1.790 | 2.253 |
| 14 | 3 | 40158347 | 7634072 | 5794 | CAACAGCTTCRTGGTGTATTT | 0.446 | 0.441 | 1.211 | 1.570 | 2.686 |
| 14 | 3 | 40170306 | 12486603 | 5795 | CCTACAGAACRTCTTCACTAT | 0.478 | 0.495 | 1.569 | 1.639 | 2.751 |
| 14 | 3 | 40180682 | 1799423 | 5796 | TGTCAGCTCTRTTTTCAATCT | 0.930 | 0.942 | 0.759 | 1.431 | 2.048 |
| 14 | 3 | 40194450 | 2255401 | 5797 | TGAATGGCCAYAGTATAGAAT | 0.681 | 0.683 | 0.775 | 1.854 | 1.975 |
| 14 | 3 | 40201689 | 2371176 | 5798 | GGAGCCTGTTMGTGCACAGTT | 0.543 | 0.544 | 1.383 | 1.365 | 2.336 |
| 14 | 3 | 40212567 | 2256927 | 5799 | GTTATGCATCMTCTAGTTTAT | 0.450 | 0.457 | 1.585 | 1.637 | 1.572 |
| 14 | 3 | 40223397 | 2371129 | 5800 | GCCTTGTAAAYTAGTCTTTCA | 1.766 | 1.771 | 1.513 | 1.838 | 1.958 |
| 14 | 3 | 40227350 | 1799422 | 5801 | TCTGCCTGCTSTCCTCCAGCA | 1.537 | 1.567 | 1.738 | 1.778 | 1.921 |
| 14 | 3 | 40232784 | 1714408 | 5802 | AAAGGCCTGGMAAGCAAGGCA | 0.516 | 0.492 | 1.746 | 1.465 | 2.264 |
| 14 | 3 | 40246701 | 1317217 | 5803 | ATGATTAATAKCTGGAGAAAG | 0.992 | 1.022 | 1.512 | 1.843 | 2.388 |
| 14 | 3 | 40249744 | 13092724 | 5804 | TTTCCTGTTCRCCCCCATGGT | 0.000 | 0.000 | 0.746 | 2.141 | 2.357 |
| 14 | 3 | 40286329 | 4974028 | 5805 | TCAATGAAGGRCAGCTGGTGA | 0.939 | 0.952 | 1.333 | 2.250 | 2.569 |
| 14 | 3 | 40301127 | 4974039 | 5806 | TCATATTTGCKTTTAGCATTA | 0.036 | 0.036 | 1.959 | 2.238 | 2.542 |
| 14 | 3 | 40320412 | 9868700 | 5807 | GGAGGCCTCTRAAGCCAAATC | 1.667 | 1.734 | 2.190 | 2.312 | 2.499 |
| 14 | 3 | 40327055 | 9826419 | 5808 | GAATGTAATCRAGATGACGCA | 2.448 | 2.367 | 2.104 | 2.619 | 2.452 |
| 14 | 3 | 40337126 | 11718621 | 5809 | TATCACATCAYTCTACATATG | 1.229 | 1.261 | 2.418 | 2.518 | 2.635 |
| 14 | 3 | 40342528 | 7652014 | 5810 | TAACAGTGGAYAAAAGCCTAT | 0.909 | 0.911 | 2.767 | 2.401 | 2.621 |
| 14 | 3 | 40349555 | 4974067 | 5811 | AAGTCTAACAYGCAGCTCTGA | 2.164 | 2.149 | 2.656 | 2.439 | 2.569 |
| 14 | 3 | 40354011 | 9821036 | 5812 | GGAGTTGTTCRGGGCAAGGAA | 2.835 | 2.815 | 2.305 | 2.669 | 2.578 |
| 14 | 3 | 40371258 | 17078511 | 5813 | GTAATTAGAARTTAACAACCA | 1.143 | 1.141 | 2.331 | 2.705 | 2.654 |
| 14 | 3 | 40378879 | 9860162 | 5814 | GGCTTGTAGTYAGCATACTTC | 1.939 | 1.966 | 2.606 | 2.568 | 2.816 |
| 14 | 3 | 40379774 | 10510708 | 5815 | TGAATCAAGTYGGTCTACAAT | 1.516 | 1.521 | 2.394 | 2.596 | 2.735 |
| 14 | 3 | 40398626 | 9854493 | 5816 | CTCTAGTCTAYAGTTAGCTAT | 2.970 | 3.031 | 2.456 | 2.768 | 2.781 |
| 14 | 3 | 40404187 | 2305521 | 5817 | ATGTTCTGAARAAAAGCCCCG | 2.236 | 2.313 | 2.511 | 2.689 | 2.772 |
| 14 | 3 | 40417584 | — | 5818 | CTGGAGAGGTSCTGAGATGTT | 0.463 | 0.453 | 3.082 | 2.580 | 2.778 |
| 14 | 3 | 40424355 | 6783755 | 5819 | TCTAAGAAGTYAGACAGACAT | 0.374 | 0.364 | 2.622 | 2.549 | 2.795 |
| 14 | 3 | 40431030 | 7645864 | 5820 | GCAAAAGGGAYTGTGGTGTAC | 1.021 | 1.048 | 2.042 | 3.057 | 2.669 |
| 14 | 3 | 40433045 | 9332450 | 5821 | TACCCTTATAYGGCAGAACTT | 2.326 | 2.340 | 1.963 | 2.718 | 2.623 |
| 14 | 3 | 40435771 | 13324109 | 5822 | AATGAACAAAYAGGTAATCTT | 1.437 | 1.410 | 2.816 | 2.748 | 2.654 |
| 14 | 3 | 40447322 | — | 5823 | ATTCACATACRGACACCTGAT | 0.412 | 0.402 | 2.552 | 2.761 | 2.613 |
| 14 | 3 | 40453155 | 6762997 | 5824 | TCTTCCTAACYCAAGCACAGG | 2.876 | 2.897 | 2.497 | 2.569 | 2.344 |
| 14 | 3 | 40462079 | 11707278 | 5825 | ATTGGCTACAWTTAACAACAA | 2.123 | 2.115 | 2.725 | 2.456 | 2.020 |
| 14 | 3 | 40481552 | 6771527 | 5826 | ACTGGTTCCCYTGGTAAGAGC | 2.217 | 2.533 | 2.480 | 2.068 | 1.824 |
| 14 | 3 | 40496322 | 4974029 | 5827 | CACTGCAGTTSTGACAACTAA | 0.931 | 0.959 | 1.993 | 2.009 | 1.831 |
| 14 | 3 | 40506140 | 6762251 | 5828 | AGATGCCTGARTATAGTAATC | 0.432 | 0.426 | 1.267 | 1.798 | 1.559 |
| 14 | 3 | 40515742 | 6599106 | 5829 | AAATCCAGAGRCATATATTTT | 0.651 | 0.633 | 0.454 | 1.099 | 1.306 |
| 14 | 3 | 40534082 | 4973907 | 5830 | GATTTTTCAASAGCAACCTTC | 0.026 | 0.025 | 0.234 | 0.710 | 1.104 |
| 14 | 3 | 40540598 | 6599108 | 5831 | AATATGAAAARTAAGTTCTTT | 0.345 | 0.344 | 0.165 | 0.291 | 1.096 |
| 14 | 3 | 40551065 | 3924444 | 5832 | TTACCATGTGYCTGAATTACA | 0.234 | 0.240 | 0.092 | 0.125 | 0.703 |
| 14 | 3 | 40574409 | 4973908 | 5833 | TGAGAGGACCRTCCAGAACAT | 0.072 | 0.073 | 0.177 | 0.103 | 0.403 |
| 14 | 3 | 40583767 | 6762428 | 5834 | TCCCTTTCTCYGAGTGGTTTT | 0.336 | 0.346 | 0.082 | 0.050 | 0.169 |
| 14 | 3 | 40592543 | 6774841 | 5835 | GTTGACTCTTYTGCTGGCTTC | 0.344 | 0.350 | 0.069 | 0.064 | 0.094 |
| 14 | 3 | 40609500 | 6599112 | 5836 | CTCTTTTTGGWTTGGTAGGCA | 0.027 | 0.028 | 0.067 | 0.048 | 0.070 |
| 14 | 3 | 40624764 | 6599113 | 5837 | GAGGTTGGCASTGTCAGATCA | 0.169 | 0.169 | 0.069 | 0.083 | 0.025 |
| 14 | 3 | 40634559 | 6772841 | 5838 | AGGATAGCCCRTCTTGGTTTG | 0.205 | 0.207 | 0.036 | 0.090 | 0.044 |
| 14 | 3 | 40647131 | 4577438 | 5839 | AGGCTTTTTGYTTTGAGGGCA | 0.294 | 0.304 | 0.177 | 0.072 | 0.052 |
| 14 | 3 | 40665380 | 4383488 | 5840 | TTGTCATCTGYGCAGTGTCCT | 0.220 | 0.216 | 0.195 | 0.026 | 0.180 |
| 14 | 3 | 40678197 | 7650783 | 5841 | TGAAAAATGGYTGCTTTCTCA | 0.625 | 0.632 | 0.186 | 0.073 | 0.289 |
| 14 | 3 | 40686361 | 6599117 | 5842 | AGGGCATACTRAGAGCCCCCA | 0.261 | 0.241 | 0.093 | 0.121 | 0.258 |
| 15 | 4 | 77038761 | 4859408 | 5843 | CTCTTTTAGTRTATCTGAGGT | 0.264 | 0.267 | 0.409 | 0.175 | 0.435 |
| 15 | 4 | 77044119 | 324719 | 5844 | ATCCTTTAAGSAGATCTGTAG | 0.166 | 0.166 | 0.211 | 0.157 | 0.409 |
| 15 | 4 | 77052318 | 3796479 | 5845 | CTCATAAATTRCATAATACCA | 0.095 | 0.094 | 0.022 | 0.223 | 0.439 |
| 15 | 4 | 77060174 | 17000863 | 5846 | GAGCTTACTTRTCGTTACGTA | 0.331 | 0.280 | 0.007 | 0.381 | 0.401 |
| 15 | 4 | 77065861 | 17000870 | 5847 | ACTTAGTCTTMCTCAACCGGC | 0.019 | 0.019 | 0.061 | 0.209 | 0.416 |
| 15 | 4 | 77076629 | — | 5848 | TACATTGTGGRTATATTTGGT | 0.029 | 0.029 | 0.530 | 0.228 | 0.421 |
| 15 | 4 | 77083718 | 11728073 | 5849 | CCTTGGATCTYTTAAGCCAGA | 0.664 | 0.690 | 0.474 | 0.407 | 0.396 |
| 15 | 4 | 77091522 | 17284996 | 5850 | ATATTAAATAYGTCTTTTTCC | 1.509 | 1.633 | 0.623 | 0.445 | 0.237 |
| 15 | 4 | 77093390 | 905954 | 5851 | GTTACCTCTCYGACCCCACAC | 0.155 | 0.153 | 0.992 | 0.458 | 0.191 |
| 15 | 4 | 77107327 | — | 5852 | GATTCTTTATYATTCTTTGTG | 0.363 | 0.365 | 0.826 | 0.676 | 0.504 |
| 15 | 4 | 77115063 | 7696758 | 5853 | ATTTCAAACTMCTCAGAGCAT | 0.946 | 0.919 | 0.298 | 0.712 | 0.526 |
| 15 | 4 | 77122212 | 17000922 | 5854 | GCAGGTGTCTYGCATTTGAGA | 0.212 | 0.207 | 0.524 | 0.474 | 0.679 |
| 15 | 4 | 77127596 | — | 5855 | CTGTTTCTCCRTGTGCTCACA | 0.323 | 0.337 | 0.398 | 0.550 | 1.221 |
| 15 | 4 | 77145093 | 6811934 | 5856 | TTAAAAAAAAYTAAACCACCT | 0.745 | 0.719 | 0.105 | 0.559 | 1.323 |
| 15 | 4 | 77157235 | 7669428 | 5857 | ACTCAAACTCRTGTTCTTAAT | 0.097 | 0.095 | 0.694 | 0.771 | 1.320 |
| 15 | 4 | 77163081 | 1976518 | 5858 | GTTCTGGCGAYGGGAGGCCAT | 0.002 | 0.002 | 0.610 | 1.217 | 0.851 |
| 15 | 4 | 77170444 | 17001165 | 5859 | GCACTACCCAYTGACGCCCAT | 1.769 | 1.768 | 0.714 | 1.319 | 0.830 |
| 15 | 4 | 77181257 | 9784511 | 5860 | CTAAGATGCTYGTGTGTGTGT | 0.162 | 0.170 | 1.892 | 1.362 | 0.892 |
| 15 | 4 | 77196287 | 6819442 | 5861 | GAAATTCCTCYGGAGGATAAA | 0.918 | 0.930 | 2.026 | 1.069 | 0.688 |
| 15 | 4 | 77204330 | 3733233 | 5862 | CCAGGCAAAAYAAAATTATGT | 2.059 | 1.991 | 1.183 | 1.027 | 0.810 |
| 15 | 4 | 77222469 | 17001237 | 5863 | TCTAGAATGCRTAAAGATCTC | 0.308 | 0.293 | 1.203 | 1.197 | 1.996 |
| 15 | 4 | 77228362 | 7154 | 5864 | TGAACTAAATRTATTCCAAAG | 0.428 | 0.442 | 0.709 | 0.649 | 1.969 |
| 15 | 4 | 77234514 | 17001277 | 5865 | CAACATACTTYAGGAGTCATC | 0.079 | 0.075 | 0.151 | 0.787 | 2.093 |
| 15 | 4 | 77239950 | 17001284 | 5866 | TTAAAGTCTTYAGGGCTATTA | 0.066 | 0.068 | 0.149 | 1.864 | 2.311 |
| 15 | 4 | 77247035 | 17506007 | 5867 | TCAAGAAAATRTGTATGCGTA | 0.599 | 0.591 | 0.223 | 1.312 | 1.784 |
| 15 | 4 | 77271152 | 6849878 | 5868 | AATGTCTGGTRTATTGAAACA | 0.285 | 0.275 | 2.116 | 1.529 | 2.412 |
| 15 | 4 | 77287534 | 7670156 | 5869 | CTGAGAGATGWAGGTGGATTA | 0.665 | 0.659 | 2.508 | 1.749 | 2.751 |
| 15 | 4 | 77291919 | 867562 | 5870 | CTGTGTATTTRAGTTGTTTCC | 4.089 | 4.568 | 2.693 | 1.826 | 2.096 |
| 15 | 4 | 77313707 | 4859415 | 5871 | TTAGTAATGCRTCTGATTGCA | 0.621 | 0.645 | 2.763 | 2.810 | 2.312 |
| 15 | 4 | 77322926 | 4859603 | 5872 | TTCTCTCTTCRACTTCTTTGT | 0.000 | 0.000 | 2.788 | 3.353 | 2.953 |
| 15 | 4 | 77338523 | 4621456 | 5873 | TTCTTCTTAASCTACTGATGT | 1.047 | 1.097 | 1.443 | 3.643 | 4.267 |
| 15 | 4 | 77352642 | 4304003 | 5874 | GACTCCTGACRAGGTACCTGA | 0.194 | 0.195 | 2.738 | 3.819 | 3.568 |
| 15 | 4 | 77359429 | — | 5875 | GAAGAGTACARTTAGGCCCAG | 1.702 | 1.743 | 2.514 | 2.491 | 3.819 |
| 15 | 4 | 77365621 | 17001359 | 5876 | GCGCACGTGTWCCATGTGTTT | 2.290 | 2.287 | 1.790 | 3.312 | 3.886 |
| 15 | 4 | 77378467 | 7654988 | 5877 | CATTTAGACTSTGACCGACAA | 0.504 | 0.498 | 2.676 | 2.924 | 3.568 |
| 15 | 4 | 77384429 | 10028141 | 5878 | AAATATTATTRACACTTCAGA | 0.381 | 0.376 | 3.139 | 2.816 | 3.102 |
| 15 | 4 | 77391640 | 17001424 | 5879 | AAACCCAACASGATGACTGGG | 2.131 | 2.037 | 2.135 | 2.879 | 2.872 |
| 15 | 4 | 77402183 | 11733489 | 5880 | TATATCTACGRTGATGCCTCT | 3.192 | 3.245 | 2.591 | 2.682 | 2.979 |
| 15 | 4 | 77416693 | 4481255 | 5881 | AGAAAATCTTYACCTTCTGTT | 0.499 | 0.503 | 2.477 | 2.459 | 3.120 |
| 15 | 4 | 77430723 | 6824251 | 5882 | CACATTCCAAMATAACAGAAC | 2.199 | 2.159 | 2.364 | 2.470 | 3.353 |
| 15 | 4 | 77439978 | 3733256 | 5883 | GGAGGTGGAGSGTTTCCCCAC | 0.305 | 0.313 | 1.847 | 2.637 | 3.267 |
| 15 | 4 | 77448185 | — | 5884 | TACCAGGAGGWAGTATGTACT | 1.896 | 1.817 | 2.092 | 3.052 | 3.007 |
| 15 | 4 | 77456563 | 17001573 | 5885 | TTATTTGGGCRTGTAGCTATG | 1.087 | 1.010 | 2.133 | 2.961 | 3.127 |
| 15 | 4 | 77467490 | 6824953 | 5886 | AGTCAGAGACSAAAGGACTTG | 0.256 | 0.251 | 3.019 | 3.293 | 3.233 |
| 15 | 4 | 77474753 | 999361 | 5887 | ACTTGGGCAGMAGCATGAAGC | 2.357 | 2.479 | 3.130 | 3.114 | 3.258 |
| 15 | 4 | 77481924 | 17001640 | 5888 | TTCAACCAGCRTAAGCTTCCA | 2.966 | 2.837 | 2.883 | 2.988 | 2.953 |
| 15 | 4 | 77488248 | 17001659 | 5889 | GCCCTGACTGYGGCCAGTTCT | 1.120 | 1.098 | 3.332 | 3.046 | 3.120 |
| 15 | 4 | 77501861 | 2034004 | 5890 | TTATCACCGCYCTCTTCCTCT | 0.321 | 0.335 | 1.902 | 2.633 | 2.593 |
| 15 | 4 | 77534206 | 17001726 | 5891 | GTGTACAAACRAAGGATATAT | 0.347 | 0.349 | 0.991 | 3.079 | 2.615 |
| 15 | 4 | 77543775 | 17236158 | 5892 | ACTGTCTTTGSTCTCTGTATT | 0.315 | 0.330 | 0.634 | 2.150 | 2.082 |
| 15 | 4 | 77550724 | 1441911 | 5893 | AGATTTAAGTKCCTAAGTTGG | 1.392 | 1.383 | 1.065 | 1.076 | 1.781 |
| 15 | 4 | 77559991 | 1530296 | 5894 | TAGTGGCAAARCACCCTTCTT | 0.364 | 0.373 | 0.983 | 0.730 | 1.909 |
| 16 | 4 | 100854365 | 28666735 | 5895 | CAATAGAGGGYTAAATTTAAA | 0.397 | 0.408 | 0.286 | 0.971 | 0.514 |
| 16 | 4 | 100861842 | 3816873 | 5896 | CTTCATCTAAYCCATGGAAAG | 0.257 | 0.262 | 0.475 | 0.901 | 0.647 |
| 16 | 4 | 100869590 | 982424 | 5897 | CTGGAAATTGYAAAGTGACCT | 0.239 | 0.234 | 0.840 | 0.672 | 0.636 |
| 16 | 4 | 100878983 | 17029215 | 5898 | TTCAAAAGTGMCAGCAGCATT | 1.510 | 1.493 | 0.994 | 0.421 | 0.822 |
| 16 | 4 | 100889431 | 2298747 | 5899 | CTTGGGAAACWGTCATTACAA | 0.827 | 0.874 | 0.928 | 0.660 | 0.704 |
| 16 | 4 | 100908302 | — | 5900 | AGCTCAGTCAWTCTTGTTTAG | 0.639 | 0.607 | 0.804 | 0.563 | 0.520 |
| 16 | 4 | 100914572 | 17029251 | 5901 | GCCAAGCTCARATCAGACTCC | 0.115 | 0.116 | 0.517 | 0.815 | 0.360 |
| 16 | 4 | 100921177 | 17600719 | 5902 | TCTCCACGCCRGCCTGTGCCC | 0.085 | 0.084 | 0.214 | 0.829 | 0.412 |
| 16 | 4 | 100929924 | 17029267 | 5903 | GTCTGATCACYGCTGTGGAGG | 0.796 | 0.834 | 0.334 | 0.374 | 0.355 |
| 16 | 4 | 100947301 | 17029309 | 5904 | TGAATCATCARGTGCTATTGT | 0.127 | 0.122 | 0.413 | 0.199 | 0.883 |
| 16 | 4 | 100958504 | 13123629 | 5905 | AGTCCACCCTMACCTAAAAAA | 0.967 | 0.993 | 0.408 | 0.142 | 0.909 |
| 16 | 4 | 100972443 | 17029332 | 5906 | TTTCCCCAGGYAGCTGAATCA | 0.306 | 0.298 | 0.177 | 0.143 | 0.559 |
| 16 | 4 | 100983727 | 10516451 | 5907 | TTTACTAACCRTCATTACAAA | 0.090 | 0.089 | 0.234 | 0.773 | 0.589 |
| 16 | 4 | 100993990 | 4699759 | 5908 | GTATTCAAATYGATCACCTAG | 0.152 | 0.153 | 0.054 | 0.637 | 0.460 |
| 16 | 4 | 101011970 | 6532828 | 5909 | CCTAGCAAGCRCTGCACGAGA | 0.293 | 0.294 | 0.733 | 0.641 | 0.531 |
| 16 | 4 | 101019807 | 7675455 | 5910 | TAGGCAAGACSTGGTATCCTA | 0.174 | 0.171 | 0.883 | 0.643 | 1.011 |
| 16 | 4 | 101026498 | 17537090 | 5911 | AATTCCATGARGACAGGTTTT | 2.394 | 2.261 | 0.875 | 0.561 | 0.828 |
| 16 | 4 | 101033818 | 1820495 | 5912 | TAAAAGTTTGYTCACCTCCCC | 0.465 | 0.455 | 1.185 | 0.677 | 1.320 |
| 16 | 4 | 101041979 | 716556 | 5913 | TACTAGCCAGRTTAAGTTCAG | 0.087 | 0.085 | 1.156 | 1.354 | 1.398 |
| 16 | 4 | 101062450 | 6831238 | 5914 | CCTCACACTGYCTCCATGGTT | 0.990 | 0.989 | 0.315 | 1.336 | 1.306 |
| 16 | 4 | 101070943 | 11735070 | 5915 | GAGGAAACAASTAGGAACAAT | 0.030 | 0.029 | 0.969 | 2.080 | 1.354 |
| 16 | 4 | 101082500 | 10031644 | 5916 | AGTAGGGAATKTATTTGCACA | 0.464 | 0.475 | 0.971 | 1.554 | 2.812 |
| 16 | 4 | 101095575 | 2282588 | 5917 | GAAAACAAGTWGGTTGAGAAA | 1.892 | 2.113 | 1.268 | 1.347 | 2.855 |
| 16 | 4 | 101103675 | 10031708 | 5918 | CAGAGCTTCTYGCATCATTTC | 0.140 | 0.143 | 2.088 | 1.269 | 3.551 |
| 16 | 4 | 101110330 | 2162386 | 5919 | AACAGGTTATMTTTCAAGGCC | 1.673 | 1.761 | 1.728 | 2.536 | 3.585 |
| 16 | 4 | 101116777 | — | 5920 | TTTGGTACGTKTTGTAGAACA | 1.136 | 1.131 | 0.820 | 2.801 | 3.434 |
| 16 | 4 | 101123160 | 4699387 | 5921 | TTTGACGACASCTACCGCTAT | 0.188 | 0.185 | 2.531 | 3.924 | 3.761 |
| 16 | 4 | 101129781 | 4699767 | 5922 | GCACCTTATCYGGGCCCAAGT | 0.232 | 0.225 | 1.924 | 3.447 | 3.924 |
| 16 | 4 | 101130928 | 11935615 | 5923 | CAGCTTGTTCRGGTCATGTTC | 3.916 | 3.585 | 3.146 | 3.102 | 4.187 |
| 16 | 4 | 101149661 | 6829912 | 5924 | GCTGAGCCTAYGGTCTGCTCT | 0.747 | 0.745 | 3.342 | 3.342 | 3.924 |
| 16 | 4 | 101158451 | 11723286 | 5925 | TTTATTTACARAGGCTATTTT | 3.996 | 3.761 | 2.961 | 3.503 | 3.503 |
| 16 | 4 | 101166087 | 11937985 | 5926 | TACTTTGATAYTATGAAAAAC | 0.327 | 0.328 | 2.708 | 3.665 | 3.966 |
| 16 | 4 | 101180236 | 11931053 | 5927 | AATGGCCAAARTAGAAGACAT | 0.859 | 0.872 | 3.434 | 3.761 | 3.761 |
| 16 | 4 | 101183482 | 17029641 | 5928 | TCTTACACATRAAGTATATGG | 1.691 | 1.656 | 1.776 | 2.633 | 3.422 |
| 16 | 4 | 101206644 | 17029667 | 5929 | CTCATCTCCARTTTGACAGAG | 1.131 | 1.165 | 1.851 | 3.322 | 3.241 |
| 16 | 4 | 101209057 | 17613664 | 5930 | AGTGTGTTCTYCAAAAAGATT | 0.816 | 0.815 | 1.655 | 2.238 | 3.187 |
| 16 | 4 | 101220468 | 17613746 | 5931 | AGCAAAAAATYAGGAAGAAAA | 0.896 | 0.890 | 2.289 | 2.277 | 2.536 |
| 16 | 4 | 101245150 | 6842840 | 5932 | TTATAATTCARTTTGCAGCCA | 0.416 | 0.430 | 1.971 | 2.415 | 2.471 |
| 16 | 4 | 101258022 | 7689566 | 5933 | AGAGCTATCARTTTATAGAGC | 3.091 | 3.139 | 1.879 | 1.853 | 1.876 |
| 16 | 4 | 101270627 | 6816635 | 5934 | AGAAATGCCARAATCTCTCAA | 0.843 | 0.898 | 1.718 | 1.515 | 1.931 |
| 16 | 4 | 101278022 | 11932783 | 5935 | TTATTTCATGKGATCCTTTCA | 0.106 | 0.110 | 1.799 | 1.428 | 2.229 |
| 16 | 4 | 101287921 | 6838495 | 5936 | TTATTTTAGAYATCTGTGGCA | 1.359 | 1.438 | 0.495 | 1.479 | 1.910 |
| 16 | 4 | 101295878 | — | 5937 | ACATTTGCCCKTAACTTTAGA | 0.067 | 0.065 | 0.530 | 1.682 | 1.666 |
| 16 | 4 | 101304621 | — | 5938 | GGTGATTAACYGCACTTGGCT | 0.056 | 0.054 | 1.103 | 1.511 | 1.610 |
| 16 | 4 | 101316549 | — | 5939 | TGTTGTCAATKAAAGGGAAAA | 0.951 | 0.986 | 1.086 | 1.441 | 1.464 |
| 16 | 4 | 101328817 | 17029724 | 5940 | TTTAGTTGCARTTAAATGCTG | 1.511 | 1.610 | 1.798 | 1.352 | 1.465 |
| 16 | 4 | 101350212 | 6532842 | 5941 | TGCTCATCTCYTCAGCTGACA | 1.276 | 1.262 | 1.782 | 1.114 | 1.014 |
| 16 | 4 | 101362457 | 7668076 | 5942 | GGTGTACAAAKGATTTTTTCA | 2.358 | 3.071 | 1.519 | 1.121 | 0.961 |
| 16 | 4 | 101376717 | 4699391 | 5943 | ACTGGAGACAYGCTCAGCCCC | 0.022 | 0.023 | 1.070 | 1.108 | 1.158 |
| 16 | 4 | 101395853 | 7671736 | 5944 | GATAATGAACRTTTTGTTATC | 0.084 | 0.079 | 0.603 | 1.061 | 1.332 |
| 16 | 4 | 101415183 | 4699777 | 5945 | CTACATATGGYATACTATTTT | 0.108 | 0.113 | 0.002 | 0.734 | 1.382 |
| 16 | 4 | 101421476 | 17029744 | 5946 | TTTTGAGCTCRTGATCATTCA | 0.121 | 0.125 | 0.056 | 0.655 | 1.560 |
| 17 | 4 | 126424833 | 1986513 | 5947 | CTTAGTTTTAWCTCCACAGAA | 0.000 | 0.000 | 0.340 | 0.605 | 0.906 |
| 17 | 4 | 126433664 | 4349615 | 5948 | TTGCCCTCTCMAGGTTTTCTT | 0.558 | 0.516 | 0.608 | 0.390 | 0.695 |
| 17 | 4 | 126443621 | 4240276 | 5949 | ACTGTGCAAARAAGTCGTGTC | 0.518 | 0.518 | 0.766 | 0.741 | 0.707 |
| 17 | 4 | 126456202 | 4532242 | 5950 | AAAAGTAAATRTAGAAATCAT | 1.070 | 1.091 | 0.678 | 0.568 | 0.572 |
| 17 | 4 | 126478116 | 11721960 | 5951 | TCATGTATAARTTTGTGTCAT | 0.390 | 0.410 | 0.957 | 0.906 | 0.473 |
| 17 | 4 | 126491841 | 4240277 | 5952 | CTCATTTTCTKAAACAGTCAA | 0.384 | 0.293 | 0.750 | 0.809 | 0.628 |
| 17 | 4 | 126504147 | 17009345 | 5953 | TCTGACCAAARGTGTGCATGA | 1.060 | 1.057 | 0.690 | 0.637 | 0.516 |
| 17 | 4 | 126518233 | 17009376 | 5954 | TCCTCTCACTSAGTTAATAGC | 0.088 | 0.091 | 0.627 | 0.645 | 0.507 |
| 17 | 4 | 126528678 | 17793037 | 5955 | AATTCACTCARGAAGTATTTA | 0.993 | 0.954 | 0.490 | 0.537 | 0.487 |
| 17 | 4 | 126539380 | 17009427 | 5956 | GTGTGGAAGARACCATACATT | 0.255 | 0.271 | 0.287 | 0.439 | 0.531 |
| 17 | 4 | 126554568 | 12512873 | 5957 | ATAGAAATTTWGGACAGTATT | 0.044 | 0.044 | 0.547 | 0.363 | 0.486 |
| 17 | 4 | 126562521 | 10518462 | 5958 | TCTAAGTTTAYGTTTTGAGAC | 0.538 | 0.530 | 0.208 | 0.191 | 0.382 |
| 17 | 4 | 126573834 | 1355167 | 5959 | AATAATTCTCYCCTTCAACAT | 0.756 | 0.745 | 0.150 | 0.333 | 0.562 |
| 17 | 4 | 126583321 | 11098804 | 5960 | TATGGTGTTAYGTCAATTGTT | 0.102 | 0.104 | 0.252 | 0.186 | 0.512 |
| 17 | 4 | 126594846 | 7677005 | 5961 | CGCTCAGACTRTCTGGATTCA | 0.038 | 0.040 | 0.300 | 0.260 | 0.313 |
| 17 | 4 | 126598940 | — | 5962 | TGAAGAAAGASAGTTTGCTAT | 0.392 | 0.298 | 0.160 | 0.576 | 0.403 |
| 17 | 4 | 126613149 | 10034706 | 5963 | AATCCCATTTRCAAAGATCCT | 0.667 | 0.674 | 0.340 | 0.468 | 0.237 |
| 17 | 4 | 126622721 | 17801021 | 5964 | TAGATGATTTSATAAAGACTT | 0.355 | 0.338 | 0.823 | 0.281 | 0.299 |
| 17 | 4 | 126628378 | 7676058 | 5965 | AAGTGAAATTKTATATGAAGT | 0.621 | 0.640 | 0.707 | 0.397 | 0.405 |
| 17 | 4 | 126634576 | 17009520 | 5966 | ATGATGATGAMAAAGTTGATT | 1.219 | 1.171 | 0.443 | 0.381 | 0.506 |
| 17 | 4 | 126643919 | 9307561 | 5967 | AGTTAGGGAAYACCGGATGAG | 0.143 | 0.137 | 0.513 | 0.451 | 0.511 |
| 17 | 4 | 126659459 | 17009559 | 5968 | AAGCAGACTGYGAGAATTCTA | 0.102 | 0.101 | 0.307 | 0.436 | 0.713 |
| 17 | 4 | 126664946 | 13146586 | 5969 | ACATTAAATGYCTTCTGTACC | 0.573 | 0.455 | 0.146 | 0.660 | 0.761 |
| 17 | 4 | 126671984 | 13146027 | 5970 | TCCGTTCATTRAGAAACATTT | 0.052 | 0.052 | 0.271 | 0.689 | 1.223 |
| 17 | 4 | 126683303 | 17009611 | 5971 | TATTGTATTCMTTTGATACGC | 0.648 | 0.643 | 0.690 | 0.640 | 1.079 |
| 17 | 4 | 126696793 | 4833325 | 5972 | ACAGTTTAATYTTATTTTCCA | 0.619 | 0.630 | 0.798 | 0.702 | 1.112 |
| 17 | 4 | 126705065 | 6842220 | 5973 | TAATGAAAATYGCTTGAAAAC | 1.061 | 1.033 | 1.293 | 1.399 | 2.245 |
| 17 | 4 | 126711912 | 1395233 | 5974 | AATAATATGGKAAGGTTGTGA | 0.826 | 0.750 | 1.043 | 1.401 | 2.040 |
| 17 | 4 | 126718936 | 9307564 | 5975 | ATTGAATGAAYGATTTTAATA | 1.073 | 1.058 | 1.758 | 1.599 | 2.206 |
| 17 | 4 | 126726483 | 13108706 | 5976 | CCTTATGGCARCTTTTCTGCA | 0.399 | 0.373 | 1.533 | 3.258 | 2.015 |
| 17 | 4 | 126732504 | 4834042 | 5977 | CTAATGGTTGRGTAGAAAGCA | 2.270 | 2.290 | 1.345 | 2.890 | 2.283 |
| 17 | 4 | 126738202 | 17009708 | 5978 | GAAAGTCTGCRGATACCATGG | 0.035 | 0.031 | 3.303 | 2.694 | 2.210 |
| 17 | 4 | 126744932 | 17009734 | 5979 | TGCTTCAGACRTTATAAAGGC | 0.315 | 0.318 | 2.988 | 2.368 | 2.207 |
| 17 | 4 | 126752525 | 12506486 | 5980 | GGACATGGGAKCAAGAGGTGA | 4.497 | 3.819 | 2.070 | 2.170 | 2.046 |
| 17 | 4 | 126759250 | 17009779 | 5981 | CACTAACTAAMCCTAATGGTT | 0.364 | 0.374 | 2.120 | 2.033 | 1.993 |
| 17 | 4 | 126765749 | 2068148 | 5982 | CAGCATAACAYACTCCAATGG | 0.361 | 0.362 | 2.185 | 1.733 | 1.975 |
| 17 | 4 | 126771743 | 7696949 | 5983 | ACTCTTTGTAYGCCCAATTGA | 0.130 | 0.126 | 0.429 | 1.748 | 2.255 |
| 17 | 4 | 126777331 | 10518469 | 5984 | TTTTAAACAGRTATTTGTCGA | 1.327 | 1.340 | 0.620 | 1.802 | 2.266 |
| 17 | 4 | 126784567 | 12643736 | 5985 | TTGCCAGCCCYAGTTCTCTAT | 0.121 | 0.119 | 0.605 | 0.542 | 2.084 |
| 17 | 4 | 126791603 | — | 5986 | AAAAACCCTARCAATGTGTTG | 0.908 | 0.884 | 0.789 | 1.035 | 2.058 |
| 17 | 4 | 126798362 | — | 5987 | GTACAATGTGRAGGAAAGTAC | 0.321 | 0.321 | 0.459 | 1.170 | 2.060 |
| 17 | 4 | 126804720 | 17009923 | 5988 | TGGGGAAAAGRGATTAAAAAT | 0.000 | 0.000 | 1.450 | 1.415 | 0.871 |
| 17 | 4 | 126817579 | 17009964 | 5989 | GCAAAGGAGARCTAGGAGGGT | 0.598 | 0.603 | 1.193 | 0.973 | 1.034 |
| 17 | 4 | 126820698 | 17818247 | 5990 | AACAAAGAATRAAAGTGCCAG | 1.692 | 1.676 | 1.529 | 1.118 | 1.240 |
| 17 | 4 | 126829646 | — | 5991 | ACAGCTCTACRTAATCGGGCA | 0.693 | 0.717 | 1.168 | 0.940 | 1.447 |
| 17 | 4 | 126839872 | 13138789 | 5992 | GCTACCCTAAYAGAGGCAGCT | 0.792 | 0.799 | 1.140 | 1.201 | 1.079 |
| 17 | 4 | 126873778 | 7441520 | 5993 | TTAACCACAASTTTGCAAAAT | 0.324 | 0.308 | 0.528 | 1.326 | 1.091 |
| 17 | 4 | 126882430 | 1864363 | 5994 | AGTCTTCTAAMCTTTCAGTCG | 0.348 | 0.338 | 0.645 | 1.414 | 0.865 |
| 17 | 4 | 126893281 | 1346715 | 5995 | TTTCAAGACTYGGTGGAAAGA | 0.427 | 0.441 | 0.799 | 0.915 | 0.817 |
| 17 | 4 | 126904971 | 6858650 | 5996 | TACCTCATAAWCCACCCCCAA | 0.937 | 0.984 | 1.060 | 0.706 | 0.786 |
| 17 | 4 | 126911493 | 9307573 | 5997 | CAGGACAGAARCCTTTTCGAG | 1.149 | 1.188 | 0.911 | 0.505 | 0.748 |
| 17 | 4 | 126921490 | 17010193 | 5998 | GACAGGAGGAYGGGATTCTCG | 0.895 | 0.911 | 0.813 | 0.453 | 0.721 |
| 17 | 4 | 126933969 | 13142551 | 5999 | GGTCTTGAGGYTGAGCAAATG | 0.134 | 0.134 | 0.480 | 0.489 | 0.680 |
| 17 | 4 | 126937821 | 17010216 | 6000 | TTCCTACTTAYGTATTTTCCA | 0.111 | 0.110 | 0.128 | 0.481 | 0.652 |
| 17 | 4 | 126949005 | 4466054 | 6001 | ACCTGCTGTAYTGGGAAGAAA | 0.117 | 0.104 | 0.052 | 0.618 | 0.679 |
| 17 | 4 | 126960644 | 17222107 | 6002 | GTTGTATCTTYGTTTTGAAGT | 0.103 | 0.100 | 0.094 | 0.440 | 0.721 |
| 17 | 4 | 126971660 | 1346716 | 6003 | GAGGCCTGAARTGCTGTATGG | 0.463 | 0.469 | 0.528 | 0.372 | 0.749 |
| 17 | 4 | 126981792 | 7697425 | 6004 | GGTAAACTTAMTATTCAGTCT | 0.398 | 0.396 | 0.741 | 0.433 | 0.623 |
| 17 | 4 | 127003431 | 7686488 | 6005 | ATATGATGAGRTGCATAAAAG | 1.526 | 1.581 | 1.082 | 0.573 | 0.455 |
| 18 | 4 | 143068047 | 1493023 | 6006 | CCAGGCCCTTMTACCTTGGTC | 0.202 | 0.189 | 0.387 | 0.562 | 0.141 |
| 18 | 4 | 143079592 | 4956411 | 6007 | CAATCTATGARCATTATCTAG | 0.000 | 0.000 | 0.362 | 0.532 | 0.124 |
| 18 | 4 | 143091542 | 4956305 | 6008 | TAGAAATTCTRGCCAATCTTT | 0.963 | 0.959 | 0.912 | 0.498 | 0.109 |
| 18 | 4 | 143100742 | 977569 | 6009 | AAAGAAGGAAYGACTAGAAAG | 0.346 | 0.362 | 0.982 | 0.406 | 0.149 |
| 18 | 4 | 143106130 | 17702304 | 6010 | ATTTTTCAGTSAATGATAATT | 1.498 | 1.494 | 0.731 | 0.364 | 0.139 |
| 18 | 4 | 143111727 | 2241911 | 6011 | TTATCAAGGTMATTTTCCTTT | 0.184 | 0.184 | 0.404 | 0.306 | 0.413 |
| 18 | 4 | 143118755 | 17645963 | 6012 | TGGCACCTGASGAAGACAGCG | 0.049 | 0.046 | 0.260 | 0.282 | 0.743 |
| 18 | 4 | 143127818 | 6843479 | 6013 | CAAGTACATCMAATAAGAACT | 0.118 | 0.116 | 0.001 | 0.127 | 0.940 |
| 18 | 4 | 143136103 | 6817546 | 6014 | TTGAGAATAARCTTCACTCTG | 0.000 | 0.000 | 0.004 | 0.424 | 0.967 |
| 18 | 4 | 143153576 | 11942550 | 6015 | CCTTTAAAATKTAAACACTTA | 0.031 | 0.030 | 0.012 | 0.608 | 0.881 |
| 18 | 4 | 143162329 | 17015129 | 6016 | TTTGCTTAACYTTTCTCATTC | 0.268 | 0.251 | 0.402 | 0.912 | 1.058 |
| 18 | 4 | 143169736 | 2218314 | 6017 | TTGTGCCATAYTTTCCTGCCT | 0.305 | 0.237 | 1.407 | 0.970 | 1.086 |
| 18 | 4 | 143180192 | 2840104 | 6018 | TCTCCTAAACSATCTCTAGGG | 1.693 | 1.733 | 1.818 | 1.020 | 0.791 |
| 18 | 4 | 143193702 | 1909868 | 6019 | GGAACTTACTRCAACAAGGCT | 2.172 | 2.290 | 1.752 | 1.568 | 0.780 |
| 18 | 4 | 143201428 | 6813602 | 6020 | GTGACCATTTYCTCACTTAAG | 1.288 | 1.350 | 1.666 | 1.606 | 0.803 |
| 18 | 4 | 143210622 | 1392779 | 6021 | CAACAAGTCCRTAAGAGTCAA | 0.316 | 0.322 | 1.954 | 1.572 | 1.266 |
| 18 | 4 | 143264681 | 168074 | 6022 | TCTCATACACRGTCTTCTAGC | 0.111 | 0.110 | 1.132 | 1.456 | 1.387 |
| 18 | 4 | 143274664 | 337237 | 6023 | CCTGAGGCTGYTGTCCTTAAA | 1.735 | 1.855 | 0.517 | 1.041 | 1.817 |
| 18 | 4 | 143299633 | 1988510 | 6024 | TCTATGGTTGYCACCACTGCT | 0.336 | 0.327 | 0.438 | 1.043 | 1.905 |
| 18 | 4 | 143305151 | 11100741 | 6025 | ATGCAGATGASTATGGTAATG | 0.022 | 0.022 | 0.469 | 0.798 | 3.358 |
| 18 | 4 | 143311890 | 4245935 | 6026 | GGGTCTCCAARGAGGCTTTTA | 0.117 | 0.114 | 0.585 | 1.219 | 3.348 |
| 18 | 4 | 143318966 | 1912714 | 6027 | TGTCCAGGAGSGTGAAGCCTT | 0.188 | 0.185 | 0.626 | 1.291 | 2.726 |
| 18 | 4 | 143325686 | 3775605 | 6028 | GCTCACCTTAYAAGGCTTATA | 2.082 | 1.983 | 1.516 | 2.213 | 2.252 |
| 18 | 4 | 143328144 | 2645800 | 6029 | GTCTGGTTTTRTGTTTGTATA | 0.457 | 0.442 | 1.574 | 2.892 | 2.467 |
| 18 | 4 | 143343738 | 336376 | 6030 | TTTGCAGTTTKAAAAAATTTA | 1.437 | 1.523 | 3.869 | 3.156 | 2.800 |
| 18 | 4 | 143351949 | 12501475 | 6031 | ATAATGATGTYATTCACACTC | 0.065 | 0.067 | 3.221 | 3.317 | 2.384 |
| 18 | 4 | 143358319 | 10519618 | 6032 | GTTGTATGTGYGGTAGGAAAT | 3.791 | 3.332 | 3.337 | 3.835 | 2.432 |
| 18 | 4 | 143364802 | 336325 | 6033 | TGACACCTGGYAACCCAAAGT | 1.119 | 1.209 | 2.245 | 2.622 | 2.469 |
| 18 | 4 | 143374465 | 336300 | 6034 | AAGTCAACATRGATTGCAAAG | 0.407 | 0.405 | 2.842 | 3.082 | 2.529 |
| 18 | 4 | 143380368 | 336330 | 6035 | GTGCAGTTTGYTAAAGAAGTC | 0.060 | 0.061 | 0.551 | 2.219 | 3.076 |
| 18 | 4 | 143390434 | 336336 | 6036 | TGTTATCTACRTTTTCTGTAA | 0.849 | 0.853 | 0.535 | 2.127 | 2.159 |
| 18 | 4 | 143403715 | 2667083 | 6037 | GCCACCCATCRGACCTCTCCA | 0.162 | 0.161 | 0.561 | 0.615 | 2.224 |
| 18 | 4 | 143413901 | 7697702 | 6038 | TCAACAGCAAYGGTAGATCCA | 1.129 | 1.110 | 0.549 | 0.570 | 1.941 |
| 18 | 4 | 143420416 | 336396 | 6039 | AAAACCTTGGYTCTCAAACAA | 0.476 | 0.469 | 0.403 | 0.496 | 2.032 |
| 18 | 4 | 143429323 | 336405 | 6040 | AACTGGCTATRAAATTTTCCT | 0.070 | 0.070 | 0.818 | 0.670 | 0.769 |
| 18 | 4 | 143439909 | 17015614 | 6041 | TTATCACCTCYTAAATGCTCT | 0.483 | 0.486 | 0.386 | 0.726 | 0.492 |
| 18 | 4 | 143448657 | 173045 | 6042 | TATTAAACAASGTCTATCAAT | 1.024 | 1.089 | 0.404 | 0.823 | 0.527 |
| 18 | 4 | 143454027 | 1219271 | 6043 | GACACCACTARGAAAAACTGA | 0.158 | 0.161 | 0.898 | 0.544 | 0.526 |
| 18 | 4 | 143460748 | 3822146 | 6044 | CTACATTTTAYTTCTCTCTGG | 0.538 | 0.520 | 0.873 | 0.421 | 0.566 |
| 18 | 4 | 143468215 | 3113519 | 6045 | CAGAAACGGCRTACAAGAAAA | 1.052 | 1.008 | 0.484 | 0.571 | 0.888 |
| 18 | 4 | 143475449 | 4574489 | 6046 | TATGAGTGGCYGGTCGGTTAC | 0.517 | 0.486 | 0.444 | 0.423 | 0.707 |
| 18 | 4 | 143489842 | 10452237 | 6047 | AAAAAAGGTAYGCATTCTGAA | 0.218 | 0.217 | 0.446 | 0.438 | 0.728 |
| 18 | 4 | 143502133 | 7672890 | 6048 | TATGTGCCAASTAGTAGATAT | 0.115 | 0.113 | 0.126 | 0.862 | 0.826 |
| 18 | 4 | 143520079 | 17015781 | 6049 | TTTTGGACCAYATCATTGTAT | 0.539 | 0.532 | 0.296 | 0.776 | 0.811 |
| 18 | 4 | 143530628 | 17654944 | 6050 | ATTCATTAACYTGATTATCAT | 0.018 | 0.016 | 0.851 | 0.650 | 0.665 |
| 18 | 4 | 143552356 | 3102439 | 6051 | TATCTATTCCSTTTCTTAAAG | 1.053 | 1.057 | 0.927 | 0.648 | 0.945 |
| 18 | 4 | 143559553 | 3113513 | 6052 | ATTATTAAAAYGTTATCCTCT | 1.568 | 1.508 | 0.994 | 0.727 | 0.895 |
| 18 | 4 | 143565954 | 2627813 | 6053 | CTATGACGTGYTTCTGAAGTT | 0.362 | 0.368 | 1.198 | 0.840 | 0.876 |
| 18 | 4 | 143572254 | 2636632 | 6054 | CTATTACTTCYGTCTTATTCT | 0.639 | 0.647 | 0.922 | 1.137 | 0.775 |
| 18 | 4 | 143579022 | 17015855 | 6055 | TTTATCTTTTWATCACTCTGA | 0.437 | 0.439 | 0.431 | 1.251 | 0.852 |
| 18 | 4 | 143585347 | 1907108 | 6056 | ATGAAAGGCTRCATCCACACT | 0.400 | 0.422 | 0.851 | 1.208 | 0.948 |
| 18 | 4 | 143592198 | 2636670 | 6057 | TGATGAGAGTRTGCATTTAGG | 0.442 | 0.445 | 0.655 | 0.658 | 0.908 |
| 18 | 4 | 143598256 | 17015920 | 6058 | AACAGAAATGYTTTGCCCTGC | 1.273 | 1.326 | 0.882 | 0.706 | 1.097 |
| 18 | 4 | 143601161 | 2635429 | 6059 | AGTATCATAAYGGTAGTGTTA | 0.264 | 0.249 | 0.737 | 0.640 | 0.930 |
| 18 | 4 | 143619115 | 2635421 | 6060 | TGTGTAACAAYGTTCCCCTTT | 1.006 | 1.010 | 0.769 | 0.595 | 0.623 |
| 18 | 4 | 143631056 | 1391093 | 6061 | ATTGGCAGTAYTAAGACAACA | 0.037 | 0.037 | 0.424 | 0.691 | 0.638 |
| 18 | 4 | 143639478 | 1497397 | 6062 | ATAATAGTCAMGATGATGGTT | 0.543 | 0.529 | 0.430 | 0.723 | 0.831 |
| 18 | 4 | 143647804 | 11938760 | 6063 | AAGGGTTCAARAAATGAGGAA | 0.407 | 0.413 | 0.334 | 0.442 | 1.556 |
| 18 | 4 | 143655337 | 17016027 | 6064 | CACTTCAAAAMGTGAGTCCCT | 0.304 | 0.307 | 0.567 | 0.487 | 1.812 |
| 18 | 4 | 143680514 | 13135641 | 6065 | AAAGCCAGTAWATGGAACGGA | 0.771 | 0.789 | 0.416 | 0.642 | 2.133 |
| 18 | 4 | 143688658 | 1489578 | 6066 | TCTGCTCCAGYAGTTCTCACT | 0.616 | 0.602 | 0.431 | 1.769 | 2.019 |
| 18 | 4 | 143695802 | 17016097 | 6067 | TCGTGGGAAAKAGTAGTTCTA | 0.196 | 0.194 | 0.965 | 2.228 | 2.573 |
| 18 | 4 | 143702078 | 3756125 | 6068 | GTCTGTAAAGYGATGAGACAT | 0.419 | 0.437 | 2.491 | 2.687 | 2.735 |
| 18 | 4 | 143712847 | 10428305 | 6069 | CAATCATCTAYAGAGACCGAG | 1.631 | 1.529 | 2.983 | 2.662 | 2.691 |
| 18 | 4 | 143718806 | 931637 | 6070 | CTGTCAGAACRTAAATTTGAT | 3.912 | 3.654 | 3.447 | 3.127 | 2.738 |
| 18 | 4 | 143739539 | 17016163 | 6071 | GATGCCCTTTSCAACACATAT | 1.868 | 1.756 | 3.397 | 3.364 | 2.730 |
| 18 | 4 | 143752462 | 3913164 | 6072 | ACTTCTGCCAYAACTTGGCTG | 2.760 | 2.658 | 3.460 | 3.187 | 2.720 |
| 18 | 4 | 143764438 | 3844178 | 6073 | TTAAGAAATARGGAACTGAAC | 0.921 | 0.914 | 2.845 | 3.114 | 2.787 |
| 18 | 4 | 143773529 | 1425537 | 6074 | TAGTCCCACAWAAATCTCATT | 3.010 | 2.879 | 2.696 | 3.073 | 2.909 |
| 18 | 4 | 143779157 | 12646243 | 6075 | TAGTAATTGTRGATTCTAGCC | 2.912 | 2.767 | 2.488 | 2.536 | 2.858 |
| 18 | 4 | 143802349 | 13107432 | 6076 | TCTTCCCACARTGTCCCTCCT | 0.683 | 0.632 | 2.425 | 2.471 | 2.789 |
| 18 | 4 | 143810140 | 10857395 | 6077 | ATAGGTTTTAMTGGAAAATTA | 0.404 | 0.408 | 1.383 | 2.061 | 2.740 |
| 18 | 4 | 143818067 | 2059513 | 6078 | ATTTGACTGASGAATTTTGCT | 0.597 | 0.593 | 0.263 | 1.779 | 2.297 |
| 18 | 4 | 143830301 | 17016354 | 6079 | ATTTCAGTGAYGTAAACCCCT | 0.063 | 0.063 | 0.151 | 0.839 | 1.899 |
| 18 | 4 | 143839071 | 16998560 | 6080 | GACAATCAGGSTCCTCTGTGG | 0.097 | 0.096 | 0.082 | 0.146 | 1.223 |
| 18 | 4 | 143845700 | 1473222 | 6081 | TAAGGACTACRTTCAGAGCCT | 0.309 | 0.277 | 0.069 | 0.146 | 0.977 |
| 18 | 4 | 143851164 | 1425523 | 6082 | CTGGTGTAGTRGTCTCTTAAG | 0.119 | 0.119 | 0.068 | 0.115 | 0.416 |
| 18 | 4 | 143865944 | 7688435 | 6083 | TATCCTGTAAYGGCCAGTGGG | 0.549 | 0.528 | 0.225 | 0.052 | 0.089 |
| 18 | 4 | 143873979 | 1364925 | 6084 | TCTCTTAGTGSTATTTCCTCT | 0.077 | 0.072 | 0.193 | 0.050 | 0.070 |
| 18 | 4 | 143886618 | 13120964 | 6085 | TTCCACTATTRCCCTTAATTC | 0.689 | 0.703 | 0.168 | 0.069 | 0.121 |
| 18 | 4 | 143896248 | 10857396 | 6086 | CAGGACAGCAYCAGAGGCAAA | 0.163 | 0.161 | 0.056 | 0.079 | 0.096 |
| 18 | 4 | 143900941 | 1443191 | 6087 | CTCCCAAAAARCTCAATGTGG | 0.084 | 0.079 | 0.071 | 0.111 | 0.136 |
| 18 | 4 | 143922224 | 1373039 | 6088 | GAGCAATTCARTGGAGGTGAA | 0.023 | 0.024 | 0.037 | 0.168 | 0.139 |
| 18 | 4 | 143933345 | 13125853 | 6089 | GCTTGATTACRTTTTTCTTGT | 0.154 | 0.148 | 0.074 | 0.220 | 0.127 |
| 18 | 4 | 143946527 | 17016550 | 6090 | AGAAGGAGGCYTCACAGTAGA | 0.515 | 0.508 | 0.290 | 0.172 | 0.141 |
| 18 | 4 | 143957383 | 1838087 | 6091 | AACGTAAGAGSATATAAAAAG | 0.345 | 0.347 | 0.380 | 0.136 | 0.134 |
| 19 | 6 | 27042873 | — | 6092 | AAACTGTGGASAAATTTCTTT | 0.000 | 0.000 | 0.001 | 0.002 | 0.462 |
| 19 | 6 | 27078381 | 9357029 | 6093 | GAGCCGGGCCRTGAGCGCCTG | 0.123 | 0.121 | 0.007 | 0.022 | 0.463 |
| 19 | 6 | 27091877 | 7752299 | 6094 | GCATTAAACCYTTCATGACTG | 0.059 | 0.057 | 0.041 | 0.177 | 1.802 |
| 19 | 6 | 27104110 | 6913271 | 6095 | CATACTGAGTSTTTTAATCCG | 0.220 | 0.211 | 0.091 | 0.351 | 1.152 |
| 19 | 6 | 27117491 | 7775041 | 6096 | TTGACTATATWCATGCATTCT | 0.324 | 0.335 | 0.531 | 0.492 | 1.522 |
| 19 | 6 | 27155895 | 994690 | 6097 | CTGAAATAACYCCTGAGGCAA | 0.591 | 0.605 | 0.879 | 2.191 | 1.267 |
| 19 | 6 | 27162476 | 2142685 | 6098 | TTTATGTTCTYTTGAAGATGA | 1.418 | 1.317 | 1.033 | 2.048 | 1.337 |
| 19 | 6 | 27172468 | — | 6099 | GTATTCACTCRAGATGTTAGT | 0.757 | 0.754 | 3.327 | 2.350 | 1.489 |
| 19 | 6 | 27183462 | 9348752 | 6100 | AGAATGTTCAYAGATATTTCT | 0.462 | 0.468 | 3.049 | 2.355 | 1.382 |
| 19 | 6 | 27201800 | 6456768 | 6101 | TGGAGGATGGSATAGTAAAGA | 4.046 | 4.011 | 3.024 | 2.137 | 1.451 |
| 19 | 6 | 27211559 | 2022272 | 6102 | ACAATTAAATRTACAGTGGTT | 0.000 | 0.000 | 2.029 | 2.150 | 1.577 |
| 19 | 6 | 27222031 | 9393790 | 6103 | TACTCAGTGTKATAAGATCAT | 0.785 | 0.758 | 1.901 | 1.807 | 1.659 |
| 19 | 6 | 27239937 | 4582379 | 6104 | TGATTAAAAGRAATTCAAGAA | 0.090 | 0.095 | 0.291 | 1.480 | 1.703 |
| 19 | 6 | 27248129 | — | 6105 | GCTTGCAGAGRTACTAAACCA | 0.284 | 0.277 | 0.370 | 1.513 | 1.630 |
| 19 | 6 | 27272736 | 6901448 | 6106 | GAAAACTAACMGTTTGAGGGT | 0.751 | 0.733 | 0.235 | 0.187 | 1.167 |
| 19 | 6 | 27281378 | 4711143 | 6107 | CTTGTCAGTTYTTAAGGACAG | 0.327 | 0.323 | 0.368 | 0.297 | 0.936 |
| 19 | 6 | 27309252 | 3800309 | 6108 | ATGATTTTTTMTCACCATGGA | 0.367 | 0.366 | 0.314 | 0.247 | 0.901 |
| 19 | 6 | 27318564 | 3800311 | 6109 | CAACAGCTTCRTAGCAAAAGG | 0.512 | 0.484 | 0.251 | 0.223 | 0.138 |
| 19 | 6 | 27324467 | 9468021 | 6110 | GTGAGCAAGTRAATAAACAAG | 0.000 | 0.000 | 0.352 | 0.201 | 0.145 |
| 19 | 6 | 27331221 | 3734577 | 6111 | CATGTGTCTGYAAACATCCTT | 0.527 | 0.519 | 0.208 | 0.133 | 0.074 |
| 19 | 6 | 27337540 | 11758636 | 6112 | TCCGATGGCTRTTCCCTCACA | 0.500 | 0.507 | 0.102 | 0.149 | 0.083 |
| 19 | 6 | 27343845 | 6915678 | 6113 | AGACCAAGAGKTCCGCTGCCA | 0.028 | 0.029 | 0.177 | 0.106 | 0.159 |
| 19 | 6 | 27364197 | 16897282 | 6114 | GCCGCTCCAGMGAGTTTAATT | 0.145 | 0.140 | 0.128 | 0.046 | 0.144 |
| 19 | 6 | 27371321 | 16897348 | 6115 | TACTGTTGCTYATACAAATCA | 0.356 | 0.345 | 0.044 | 0.066 | 0.112 |
| 19 | 6 | 27379322 | 7771953 | 6116 | ACAGAACGCGRATCCGTTGGA | 0.399 | 0.390 | 0.049 | 0.113 | 0.090 |
| 19 | 6 | 27385644 | 2281023 | 6117 | TGGGAAAGGTRGCCTTGAGCT | 0.101 | 0.099 | 0.073 | 0.133 | 0.078 |
| 19 | 6 | 27400907 | 16897710 | 6118 | TTTGTGTATGKACTTCTTCTC | 0.073 | 0.073 | 0.226 | 0.133 | 0.136 |
| 19 | 6 | 27406608 | 9379978 | 6119 | GAGGAAAACCRAAGTCACTAG | 0.281 | 0.272 | 0.300 | 0.138 | 0.128 |
| 19 | 6 | 27412565 | 6903160 | 6120 | CAATTTTTACWGTTTTTCCAA | 0.941 | 0.862 | 0.298 | 0.153 | 0.087 |
| 19 | 6 | 27428723 | 2393926 | 6121 | CTGATCACCCSAACAAAAAGG | 0.631 | 0.615 | 0.327 | 0.206 | 0.165 |
| 19 | 6 | 27438664 | 12213055 | 6122 | TATTCTGTCTYTTACAAGAAA | 0.045 | 0.046 | 0.370 | 0.303 | 0.301 |
| 19 | 6 | 27452810 | 6456774 | 6123 | AGGGAATTGTSAACAACTGAG | 0.216 | 0.177 | 0.274 | 0.323 | 0.439 |
| 20 | 6 | 152286110 | 1709183 | 6124 | TTCCCTTGTAYCGTTGTGGCT | 0.172 | 0.173 | 0.597 | 0.636 | 1.021 |
| 20 | 6 | 152300836 | 1913474 | 6125 | AAACCTTCTTRGAACCCTAGG | 1.921 | 1.863 | 0.446 | 0.745 | 1.033 |
| 20 | 6 | 152316850 | 9322341 | 6126 | ATTGCTAGACYTAATCATCCA | 0.057 | 0.057 | 0.737 | 0.479 | 1.131 |
| 20 | 6 | 152327453 | 12204714 | 6127 | TCAGGGCTCAYAATTTCTTTG | 0.023 | 0.022 | 1.100 | 0.952 | 0.787 |
| 20 | 6 | 152355742 | 6915819 | 6128 | CTTAAAATATRTAGTAAGTGC | 0.852 | 0.855 | 0.187 | 1.087 | 0.700 |
| 20 | 6 | 152369037 | 3020391 | 6129 | TAAACTCATGRCTCGGGGCTT | 0.757 | 0.789 | 0.846 | 1.287 | 0.715 |
| 20 | 6 | 152378739 | 985694 | 6130 | ATATAATACAYCCCTGAAGTT | 0.006 | 0.006 | 1.133 | 0.471 | 0.657 |
| 20 | 6 | 152392741 | 9340939 | 6131 | GTCTCATAATMATAAGAGAGT | 1.534 | 0.856 | 0.939 | 0.544 | 0.662 |
| 20 | 6 | 152402727 | 6916218 | 6132 | CAAAAGTATTWAAAAAAATCA | 0.510 | 0.514 | 0.558 | 0.817 | 0.675 |
| 20 | 6 | 152408861 | 17081994 | 6133 | TTCTATTTTCYAGTTAGACTT | 0.000 | 0.000 | 0.685 | 0.569 | 0.315 |
| 20 | 6 | 152424244 | 9340969 | 6134 | AACCACAATAYTATCAGTAAT | 0.059 | 0.065 | 0.382 | 0.439 | 0.322 |
| 20 | 6 | 152431729 | 7755185 | 6135 | TGTGTTTTTGRTTCACATGAA | 0.234 | 0.244 | 0.235 | 0.507 | 0.322 |
| 20 | 6 | 152435877 | 3020411 | 6136 | CAGTTTTTTGRGAAGTAGAGG | 0.753 | 0.812 | 0.217 | 0.180 | 0.259 |
| 20 | 6 | 152450293 | 2982712 | 6137 | TGTATTGCAGYGCCAATTGCT | 0.131 | 0.133 | 0.271 | 0.090 | 0.222 |
| 20 | 6 | 152459232 | 3020364 | 6138 | CTGGAATAACRCCTGAGATCT | 0.242 | 0.254 | 0.211 | 0.044 | 0.248 |
| 20 | 6 | 152472491 | 9397079 | 6139 | ATTTAAACATRAAAGCATGGA | 0.264 | 0.268 | 0.033 | 0.080 | 0.071 |
| 20 | 6 | 152477350 | 3778080 | 6140 | AAAACCCGGAMTCATCCATAG | 0.107 | 0.110 | 0.016 | 0.110 | 0.054 |
| 20 | 6 | 152500240 | 750686 | 6141 | TTCAACTCACRTAATGAGAAG | 0.037 | 0.037 | 0.028 | 0.040 | 0.079 |
| 20 | 6 | 152514449 | 2747648 | 6142 | TTGTTGGATAYTGAATGACAG | 0.004 | 0.004 | 0.071 | 0.047 | 0.166 |
| 20 | 6 | 152525016 | 910416 | 6143 | CTGCTTTACAYGTGGTCTCAG | 0.443 | 0.468 | 0.099 | 0.048 | 0.159 |
| 20 | 6 | 152533353 | 2813549 | 6144 | TCTTCAGTGAYGGGAGATGCC | 0.531 | 0.530 | 0.144 | 0.091 | 0.118 |
| 20 | 6 | 152542014 | 9397486 | 6145 | GATTAAACACRAGATATCTCA | 0.201 | 0.198 | 0.237 | 0.236 | 0.169 |
| 20 | 6 | 152550188 | 2813566 | 6146 | AGGATGGCTARGTAAAAGACA | 0.154 | 0.150 | 0.293 | 0.289 | 0.182 |
| 20 | 6 | 152556953 | 2256135 | 6147 | GTATCTGAAARCGGCTATCAT | 0.332 | 0.330 | 0.425 | 0.394 | 0.269 |
| 20 | 6 | 152563317 | 2813493 | 6148 | ATATCCCGATSATTTTAATTT | 0.607 | 0.620 | 0.443 | 0.431 | 0.485 |
| 20 | 6 | 152570021 | 6937954 | 6149 | ACCAAGGGCCWGTATATATGC | 0.957 | 0.962 | 0.557 | 0.388 | 0.482 |
| 20 | 6 | 152576877 | 4870079 | 6150 | GAGATATTCTRTCTTTGCTAC | 0.310 | 0.296 | 0.705 | 0.558 | 1.080 |
| 20 | 6 | 152584631 | 2763031 | 6151 | CAGCAAATACKGGAGTGACTG | 0.446 | 0.455 | 0.577 | 0.928 | 1.282 |
| 20 | 6 | 152590550 | 2247922 | 6152 | TGTGATTTAASTGACGATGAA | 0.621 | 0.622 | 0.531 | 0.840 | 1.275 |
| 20 | 6 | 152601623 | 2635430 | 6153 | GCCTTTCCACSAATCTTTCCC | 0.350 | 0.373 | 0.918 | 1.504 | 1.466 |
| 20 | 6 | 152615664 | 2253407 | 6154 | CAATATGAAGKTATGCCCACA | 0.815 | 0.834 | 0.791 | 1.660 | 1.694 |
| 20 | 6 | 152621374 | 3798756 | 6155 | GGCTGTCAATRATCCCCCGCC | 1.280 | 1.321 | 1.748 | 1.714 | 1.854 |
| 20 | 6 | 152627649 | 10872683 | 6156 | TTTGTCAGTGYTATGGGAAAG | 0.029 | 0.029 | 2.190 | 2.014 | 1.690 |
| 20 | 6 | 152641700 | 12528883 | 6157 | AGGTGTCTCCSTGTTAAATAG | 2.670 | 2.410 | 2.070 | 1.925 | 1.340 |
| 20 | 6 | 152647226 | 2296254 | 6158 | AAACAAGTAGYGATGTTCAAA | 1.339 | 1.328 | 1.940 | 2.146 | 1.343 |
| 20 | 6 | 152652592 | 4869757 | 6159 | GCAGATAGCCMAATGTACACA | 0.350 | 0.345 | 2.256 | 1.912 | 1.256 |
| 20 | 6 | 152662388 | 17215781 | 6160 | TTTCTCTTCTRGGAAAAGGCA | 0.548 | 0.575 | 0.908 | 1.201 | 1.238 |
| 20 | 6 | 152667818 | 7763242 | 6161 | ATATTCTTTCKCAATGTGTTT | 0.485 | 0.488 | 0.419 | 1.220 | 1.505 |
| 20 | 6 | 152680602 | 9383983 | 6162 | CCTGTATATCYTCTCAGTTGA | 0.684 | 0.678 | 0.290 | 0.406 | 1.727 |
| 20 | 6 | 152691531 | — | 6163 | CAGCATATTCWGCAGCAACAG | 0.233 | 0.228 | 0.234 | 0.219 | 1.392 |
| 20 | 6 | 152706238 | 630548 | 6164 | TGCATTTTAAMTTGACACATT | 0.039 | 0.037 | 0.106 | 0.387 | 1.464 |
| 20 | 6 | 152716034 | 674724 | 6165 | TTTAATTTCAYTTGCTTTTGA | 0.388 | 0.381 | 0.092 | 0.734 | 0.941 |
| 20 | 6 | 152724385 | 12206761 | 6166 | GCTCCTGCTCRGTCAAGCCCA | 0.027 | 0.027 | 0.350 | 0.645 | 0.892 |
| 20 | 6 | 152733065 | 1472023 | 6167 | TAGGAAAATTRATCCAGAGAA | 0.606 | 0.556 | 1.055 | 0.551 | 1.567 |
| 20 | 6 | 152739795 | 2306916 | 6168 | AGCTGTAACAWTTCTTGGGCA | 1.111 | 1.116 | 1.057 | 0.794 | 1.449 |
| 20 | 6 | 152745878 | 725235 | 6169 | ATGTGCTTTCYTTTGTGAAGA | 1.744 | 1.689 | 1.197 | 1.248 | 2.080 |
| 20 | 6 | 152752424 | 4870095 | 6170 | TCAATCTCACRTGAAACATTC | 0.213 | 0.210 | 1.397 | 1.981 | 2.116 |
| 20 | 6 | 152758998 | 7738089 | 6171 | AAAACTCTTTKTAGTGTCATT | 0.375 | 0.375 | 1.447 | 1.893 | 2.340 |
| 20 | 6 | 152767168 | 6905339 | 6172 | AACAATGTGAYACAAACTGTA | 1.031 | 0.950 | 1.613 | 2.495 | 3.130 |
| 20 | 6 | 152774905 | 6557214 | 6173 | CAGGCTGACCRATGATCATGC | 1.147 | 1.201 | 1.751 | 2.636 | 3.034 |
| 20 | 6 | 152781309 | 9371592 | 6174 | AAAAATGGGTRTAAACATAGA | 2.447 | 2.328 | 2.676 | 2.278 | 3.237 |
| 20 | 6 | 152789820 | 214955 | 6175 | GAAAAGGTGAYGCAAAAATTT | 0.630 | 0.617 | 2.564 | 3.460 | 3.559 |
| 20 | 6 | 152810619 | 549981 | 6176 | TCCTCAGGCCYTTTCTTTTCC | 2.950 | 2.714 | 2.634 | 3.623 | 3.988 |
| 20 | 6 | 152819217 | 17082645 | 6177 | CATAGCCCAAYTACTACTTGA | 0.581 | 0.593 | 3.245 | 3.474 | 3.676 |
| 20 | 6 | 152828926 | 761408 | 6178 | TGTTTAAAAAYAGATAATAAG | 0.670 | 0.668 | 3.392 | 3.403 | 3.988 |
| 20 | 6 | 152838247 | 6909684 | 6179 | TATATTAAAAYGAAATGGACT | 3.292 | 3.011 | 1.824 | 3.835 | 4.153 |
| 20 | 6 | 152848308 | 17082676 | 6180 | ATATAAAGTCYTCAAAATTTC | 0.000 | 0.000 | 1.696 | 3.905 | 3.748 |
| 20 | 6 | 152854769 | 214996 | 6181 | GTAAAGTCTTRGAAATGTAAA | 0.783 | 0.754 | 2.876 | 3.403 | 3.905 |
| 20 | 6 | 152861412 | 17082700 | 6182 | GTATCCCATAYAGCAAGAAGT | 0.520 | 0.532 | 2.301 | 3.835 | 3.676 |
| 20 | 6 | 152868292 | 214970 | 6183 | GTGACACAGTRTCTATACTAT | 3.367 | 3.258 | 3.130 | 3.595 | 4.225 |
| 20 | 6 | 152875047 | 579464 | 6184 | CTTAACTTCCRGATTCCACAG | 1.791 | 1.733 | 3.698 | 3.633 | 3.835 |
| 20 | 6 | 152887970 | 7755437 | 6185 | ATTCATAATTMCTTATATTGA | 0.000 | 0.000 | 3.481 | 3.869 | 3.869 |
| 20 | 6 | 152898003 | 9397512 | 6186 | TGTTGCCATAYCACTGTGTAG | 4.278 | 3.654 | 3.034 | 4.312 | 3.869 |
| 20 | 6 | 152913124 | 9479327 | 6187 | AATTTATTTTYTGGATGATTA | 0.000 | 0.000 | 3.428 | 3.869 | 3.496 |
| 20 | 6 | 152925152 | 11155857 | 6188 | CCAACAAAATRGACTTACATG | 1.043 | 1.023 | 3.723 | 3.206 | 3.676 |
| 20 | 6 | 152934088 | 17699371 | 6189 | CTGTTAGACAYGACACAGTCT | 0.217 | 0.206 | 0.689 | 2.672 | 4.568 |
| 20 | 6 | 152940828 | 11155858 | 6190 | AGCCATATACRGTCATTAAAA | 1.573 | 1.505 | 0.811 | 2.550 | 4.568 |
| 20 | 6 | 152941489 | 2141151 | 6191 | GCGACCTCTCMTAAACTTTGT | 0.061 | 0.062 | 0.531 | 0.566 | 3.676 |
| 20 | 6 | 152956242 | 11968876 | 6192 | AATATAAATTSGCATAATATG | 0.296 | 0.312 | 0.789 | 1.289 | 3.698 |
| 20 | 6 | 152964386 | 9397523 | 6193 | CAATGATCGGMTAGTAAAGTA | 0.429 | 0.431 | 0.231 | 2.386 | 3.262 |
| 20 | 6 | 152979559 | 2623964 | 6194 | CAAGCTGAAGYGAGCAACTAG | 0.789 | 0.744 | 1.137 | 2.424 | 1.633 |
| 20 | 6 | 152989528 | 7749912 | 6195 | TAAGGAATTASTAGGCATTTA | 0.245 | 0.251 | 2.494 | 1.852 | 1.802 |
| 20 | 6 | 152997160 | 2178009 | 6196 | ACTCAACATTRCCTAAAAACT | 1.999 | 1.908 | 2.240 | 1.735 | 1.873 |
| 20 | 6 | 153003053 | 17701297 | 6197 | AACCTTAGCTRGATCCATTTT | 2.910 | 2.696 | 2.287 | 1.709 | 2.276 |
| 20 | 6 | 153011820 | 10457880 | 6198 | TCCCATAATGWATCAGCTTCA | 0.042 | 0.042 | 2.208 | 1.676 | 2.172 |
| 20 | 6 | 153031553 | 1744387 | 6199 | AATCAGTGTARTTCTTGGCAA | 0.788 | 0.826 | 1.319 | 1.989 | 2.278 |
| 20 | 6 | 153037891 | 1407488 | 6200 | TTTAAATTGAKTAAAGTTCCA | 0.271 | 0.268 | 0.216 | 3.004 | 2.837 |
| 20 | 6 | 153044292 | 9383634 | 6201 | TGACACAGAGRCTATTTTTGA | 0.178 | 0.178 | 0.626 | 2.203 | 2.792 |
| 20 | 6 | 153058027 | — | 6202 | CCTTACGTGTRTGTAGTGCAT | 0.483 | 0.465 | 1.025 | 1.082 | 2.516 |
| 20 | 6 | 153070069 | 2984656 | 6203 | ATGGAGGAGAYACTAATGAAG | 1.076 | 1.052 | 1.330 | 1.620 | 2.803 |
| 20 | 6 | 153078353 | 2758768 | 6204 | TTAATTTACCYTGTCTGATAC | 1.525 | 1.458 | 1.659 | 1.659 | 2.258 |
| 20 | 6 | 153083723 | 873889 | 6205 | CTGAGAACTCYGGGCTGTTTC | 0.938 | 0.919 | 1.930 | 1.611 | 1.436 |
| 20 | 6 | 153090503 | 2758774 | 6206 | TTCAGCCCTGMTCTCCATTTT | 0.739 | 0.748 | 2.076 | 1.660 | 1.534 |
| 20 | 6 | 153097114 | 2758775 | 6207 | AAAACTCAACWTATGGAAGGC | 1.391 | 1.785 | 1.537 | 1.806 | 1.422 |
| 20 | 6 | 153109354 | 1936726 | 6208 | CAATCAGGTARCCTTTAATAA | 1.279 | 1.257 | 1.173 | 1.664 | 1.403 |
| 20 | 6 | 153116791 | 2078355 | 6209 | AAAGGCATCARTACAGTTCCC | 0.158 | 0.154 | 1.552 | 1.478 | 1.486 |
| 20 | 6 | 153135149 | 17710008 | 6210 | TTACACCCACRGCCTCAACAG | 0.348 | 0.340 | 1.001 | 1.193 | 1.487 |
| 20 | 6 | 153140084 | 9384043 | 6211 | CACATGATACYGGACAGCCAG | 1.837 | 1.743 | 0.691 | 1.054 | 1.447 |
| 20 | 6 | 153150872 | 1282450 | 6212 | GGGATCTAGGWGGGGAACAAG | 0.134 | 0.122 | 0.751 | 0.745 | 1.058 |
| 20 | 6 | 153159957 | 601240 | 6213 | TTTCTCTATASCCTTCTTTTT | 0.498 | 0.513 | 0.636 | 0.470 | 0.774 |
| 20 | 6 | 153166436 | 3823082 | 6214 | GATAACTAAAYAAAATGGTTC | 0.263 | 0.266 | 0.128 | 0.574 | 0.649 |
| 20 | 6 | 153172175 | 688136 | 6215 | TGAAGGAAAAYGATACGCAAC | 0.071 | 0.075 | 0.246 | 0.457 | 0.345 |
| 20 | 6 | 153183689 | 311347 | 6216 | TTTTTCATGTRTAATCCCAAA | 0.543 | 0.508 | 0.226 | 0.069 | 0.177 |
| 20 | 6 | 153199759 | 610111 | 6217 | AGTAATGCTCWTTCCTGCTAT | 0.467 | 0.489 | 0.137 | 0.097 | 0.182 |
| 20 | 6 | 153205685 | 311353 | 6218 | ATATATTGGCYATCTACATTG | 0.446 | 0.454 | 0.136 | 0.049 | 0.169 |
| 20 | 6 | 153213019 | — | 6219 | GCTGTATAGTRGCAAAGCTTC | 0.000 | 0.000 | 0.094 | 0.048 | 0.019 |
| 20 | 6 | 153219459 | 625764 | 6220 | TCGTTTGATGRCTTTAACAAA | 0.121 | 0.111 | 0.029 | 0.059 | 0.020 |
| 20 | 6 | 153227029 | 628410 | 6221 | GGTCATTTTARAGACTATTAT | 0.278 | 0.280 | 0.021 | 0.027 | 0.070 |
| 20 | 6 | 153235164 | 1328395 | 6222 | GCTCCTCTCTYCTCAAAGGGA | 0.034 | 0.035 | 0.047 | 0.009 | 0.081 |
| 20 | 6 | 153245407 | 9371268 | 6223 | TGTCGATGTTYTGAATCACAT | 0.381 | 0.376 | 0.052 | 0.004 | 0.112 |
| 20 | 6 | 153255860 | 1154023 | 6224 | AGAAAGTTTCRAAGGGGATTT | 0.292 | 0.258 | 0.023 | 0.106 | 0.141 |
| 20 | 6 | 153266113 | 9384046 | 6225 | CCAACACCATYGGCTCTTCTG | 0.098 | 0.101 | 0.036 | 0.172 | 0.185 |
| 20 | 6 | 153272074 | 17083127 | 6226 | ATGGGAGCATYTTATAATGTG | 0.059 | 0.061 | 0.250 | 0.194 | 0.165 |
| 20 | 6 | 153277784 | 9371269 | 6227 | GCTAGAATGCYATGGTGGATG | 0.146 | 0.150 | 0.356 | 0.371 | 0.199 |
| 20 | 6 | 153283388 | 17083144 | 6228 | AAATAGCCCAYGCAATTACAA | 1.280 | 1.287 | 0.507 | 0.495 | 0.246 |
| 21 | 7 | 30326902 | 38433 | 6229 | GATTACTGCAWCAATTCCTAG | 0.133 | 0.139 | 0.160 | 0.668 | 0.348 |
| 21 | 7 | 30332916 | 38437 | 6230 | ACCAATCCCGYGGACAATAAG | 0.039 | 0.042 | 0.171 | 0.454 | 0.470 |
| 21 | 7 | 30344024 | 38450 | 6231 | GCTCAAATATAMCTTCATTAA | 0.633 | 0.654 | 0.488 | 0.262 | 0.396 |
| 21 | 7 | 30359460 | 38466 | 6232 | CTATCATTAAWCTCTCCTTAA | 0.307 | 0.300 | 0.558 | 0.239 | 0.461 |
| 21 | 7 | 30363462 | 17159202 | 6233 | AAAATTGAAAYCCATAATGAA | 1.250 | 1.281 | 0.558 | 0.338 | 0.348 |
| 21 | 7 | 30378807 | 10227389 | 6234 | ATGGGATGAGYTCTCTCAGAC | 0.340 | 0.363 | 0.369 | 0.347 | 0.272 |
| 21 | 7 | 30398623 | 10243040 | 6235 | CAAGTCTCTTSATGCCTCTTC | 0.075 | 0.077 | 0.582 | 0.472 | 0.341 |
| 21 | 7 | 30409656 | 17159267 | 6236 | CTATGTCAGCYGAAAAATAAT | 0.241 | 0.242 | 0.183 | 0.425 | 0.364 |
| 21 | 7 | 30417902 | — | 6237 | ATGCATGTACRGCFGTCAATA | 0.740 | 0.760 | 0.240 | 0.480 | 0.427 |
| 21 | 7 | 30427347 | 6945035 | 6238 | TTATATGGGCRCAGAAGATGT | 0.183 | 0.185 | 0.375 | 0.309 | 0.481 |
| 21 | 7 | 30435477 | 10249885 | 6239 | TGGCATTATARCCTGAAAAAA | 0.541 | 0.515 | 0.457 | 0.329 | 0.464 |
| 21 | 7 | 30444327 | 14270 | 6240 | CTGCTGTAGTYGCTCCATTCA | 0.482 | 0.466 | 0.416 | 0.448 | 0.430 |
| 21 | 7 | 30462973 | 17159316 | 6241 | GGTTAAACAGRGACCCTAAGG | 0.000 | 0.000 | 0.569 | 0.467 | 0.175 |
| 21 | 7 | 30467015 | 4722999 | 6242 | TGGCTCTACCYGGTGCCATTT | 0.641 | 0.664 | 0.592 | 0.393 | 0.243 |
| 21 | 7 | 30481779 | 2008003 | 6243 | CCCAGGTGGGMTCTAATTTGA | 0.000 | 0.000 | 0.457 | 0.379 | 0.625 |
| 21 | 7 | 30489883 | 2267716 | 6244 | AGAAGGAAAAYGGACCATGGC | 0.000 | 0.000 | 0.506 | 0.228 | 0.885 |
| 21 | 7 | 30502912 | 17159371 | 6245 | TCTCCTAAAAMATCATCAGAG | 0.316 | 0.314 | 0.233 | 0.316 | 1.563 |
| 21 | 7 | 30505347 | 255105 | 6246 | AACTGGACATYGGAGCGGAAT | 0.568 | 0.568 | 0.124 | 0.753 | 1.616 |
| 21 | 7 | 30520933 | 255131 | 6247 | TACGTTCTTASGGATCCAGCC | 0.203 | 0.153 | 0.249 | 0.851 | 1.951 |
| 21 | 7 | 30535092 | 255152 | 6248 | TAATACCATCRTCAGAAATCC | 0.014 | 0.014 | 0.789 | 1.651 | 2.035 |
| 21 | 7 | 30540960 | 255160 | 6249 | ATGAAAGCTGYCTTGCCTAAG | 0.768 | 0.722 | 0.940 | 1.519 | 1.981 |
| 21 | 7 | 30552965 | 17159410 | 6250 | CTCACCTGGARAGAAAAGCTG | 1.693 | 1.726 | 2.390 | 2.036 | 2.968 |
| 21 | 7 | 30565088 | 4723010 | 6251 | CTACAGCTTCRATGGCAGCCC | 0.858 | 0.873 | 2.590 | 2.270 | 3.481 |
| 21 | 7 | 30579153 | 3901848 | 6252 | TGTCCTTCCARTGTAAAGTCC | 2.165 | 2.230 | 2.426 | 2.351 | 3.358 |
| 21 | 7 | 30592754 | 6949578 | 6253 | TTGAAAACTAYGCAAGTACAT | 0.303 | 0.302 | 2.012 | 3.511 | 3.905 |
| 21 | 7 | 30602279 | 11768076 | 6254 | TGTGGCCTTTRAGTACCTTGG | 1.690 | 1.617 | 1.882 | 3.988 | 4.153 |
| 21 | 7 | 30610787 | 7803974 | 6255 | AGCAGGGCTAYGTTCTGAGAA | 1.004 | 1.027 | 2.837 | 3.542 | 4.312 |
| 21 | 7 | 30618224 | 1990011 | 6256 | TGGATATGTCSAGTGGAGCTT | 0.552 | 0.565 | 3.835 | 3.835 | 4.312 |
| 21 | 7 | 30629046 | 10237363 | 6257 | CCCGGGTCTCRTATGCCAAAT | 3.996 | 3.576 | 3.496 | 3.775 | 4.422 |
| 21 | 7 | 30641902 | 17159487 | 6258 | GTAAGTTAACKAAGGTCTCCA | 2.330 | 2.254 | 3.676 | 4.153 | 4.312 |
| 21 | 7 | 30649882 | 10229281 | 6259 | TGGGAATAGARCTGCTTCATC | 1.197 | 1.159 | 3.511 | 3.723 | 4.225 |
| 21 | 7 | 30671937 | 17159526 | 6260 | TCAGGTTGTTSCAATGTCCCA | 3.110 | 2.896 | 3.415 | 3.710 | 3.988 |
| 21 | 7 | 30682866 | 17159567 | 6261 | CAAAGTTATAMATCTCTTAAA | 0.327 | 0.340 | 3.019 | 3.687 | 3.748 |
| 21 | 7 | 30688503 | 10216063 | 6262 | GCATTACAACRTGTGCCAGGT | 2.161 | 2.894 | 2.797 | 3.102 | 3.163 |
| 21 | 7 | 30701508 | 10281903 | 6263 | CAATCGGGGARCGATTCGATT | 0.547 | 0.570 | 1.020 | 2.687 | 3.085 |
| 21 | 7 | 30710418 | 1000597 | 6264 | AGGACAGTTTYGTAATCAGGA | 0.175 | 0.183 | 0.932 | 2.397 | 3.031 |
| 21 | 7 | 30719606 | 763422 | 6265 | ACTGGTGGCCYGCTAAATCCG | 0.237 | 0.244 | 0.408 | 0.943 | 2.669 |
| 21 | 7 | 30732804 | 765839 | 6266 | TCAGCAGTCASGAAGGCACAT | 0.218 | 0.220 | 0.432 | 0.999 | 2.127 |
| 21 | 7 | 30745035 | 10244884 | 6267 | TTGGAGCTATYGACTGATTTT | 1.090 | 1.087 | 0.464 | 0.331 | 1.819 |
| 21 | 7 | 30762253 | 7801005 | 6268 | GCCAGGCTAAYAAGGTCTCAG | 0.630 | 0.630 | 0.572 | 0.631 | 0.767 |
| 21 | 7 | 30768151 | 4723034 | 6269 | TAGACAAAATKTTAAAGTTAA | 0.221 | 0.222 | 0.534 | 0.698 | 0.805 |
| 21 | 7 | 30775780 | 17159769 | 6270 | ATGATGTGATYGGGCTGTAAA | 0.525 | 0.509 | 0.757 | 0.615 | 0.356 |
| 21 | 7 | 30776153 | 17159772 | 6271 | CCCCTTTTGCYGTGAGAGTTT | 0.123 | 0.126 | 0.647 | 0.553 | 0.316 |
| 21 | 7 | 30790343 | 4988505 | 6272 | AAGCTGAACCSGAATGTTTCC | 1.552 | 1.558 | 0.557 | 0.377 | 0.397 |
| 21 | 7 | 30797413 | 11771444 | 6273 | CGGAATGGGGYTGATCTCAGT | 0.383 | 0.387 | 0.390 | 0.296 | 0.460 |
| 21 | 7 | 30809401 | 6948808 | 6274 | CCCATTCAATYCACAATAGCG | 0.048 | 0.049 | 0.514 | 0.330 | 0.429 |
| 21 | 7 | 30818744 | 7786414 | 6275 | TCCGAACGTAYCTTTGGTGAA | 0.026 | 0.026 | 0.098 | 0.362 | 0.281 |
| 21 | 7 | 30827485 | 2391936 | 6276 | TATATATAGAYTGTATGTTAC | 0.000 | 0.000 | 0.067 | 0.469 | 0.283 |
| 21 | 7 | 30838336 | — | 6277 | TAAATGCACAYATAAAATCAT | 0.319 | 0.307 | 0.193 | 0.126 | 0.347 |
| 21 | 7 | 30848961 | 17159844 | 6278 | TGCTGGACATYTCACTGTTAC | 0.328 | 0.324 | 0.378 | 0.097 | 0.347 |
| 21 | 7 | 30858437 | 4439020 | 6279 | GGACTTGAGCYAATTACATGA | 0.643 | 0.584 | 0.246 | 0.182 | 0.457 |
| 21 | 7 | 30876872 | 17159871 | 6280 | GTTTTGAGTTRAAGGGACGGC | 0.562 | 0.565 | 0.253 | 0.326 | 0.195 |
| 21 | 7 | 30880688 | 17723231 | 6281 | CTAAGGACAGYGTATGGGATT | 0.081 | 0.080 | 0.357 | 0.348 | 0.218 |
| 21 | 7 | 30895706 | 2267729 | 6282 | TCGGGCAGAGWTACCCATCCC | 0.277 | 0.279 | 0.356 | 0.465 | 0.288 |
| 21 | 7 | 30911743 | 2284225 | 6283 | ATTTGCATCARGTCCAATCTG | 0.618 | 0.616 | 0.347 | 0.400 | 0.317 |
| 21 | 7 | 30921519 | 1541518 | 6284 | GGCCCTGCACKTAAGTGATTT | 0.602 | 0.592 | 0.610 | 0.392 | 0.262 |
| 21 | 7 | 30930958 | 10264624 | 6285 | TGTTTCAAACYGAGCAAAACA | 0.246 | 0.379 | 0.539 | 0.364 | 0.211 |
| 21 | 7 | 30936040 | 12672673 | 6286 | ATTGTAAACASTAACATAACT | 0.667 | 0.697 | 0.516 | 0.377 | 0.168 |
| 21 | 7 | 30949204 | 7785534 | 6287 | GCAAATAGGGRTAACAGCATT | 0.100 | 0.106 | 0.443 | 0.345 | 0.333 |
| 22 | 7 | 32696611 | 12154586 | 6288 | CAAATGTTTAYTGAATACTAA | 0.634 | 0.642 | 0.815 | 0.953 | 0.829 |
| 22 | 7 | 32708674 | — | 6289 | ATGTTTCCCAMAAAGACAATC | 1.663 | 1.571 | 1.446 | 0.883 | 0.575 |
| 22 | 7 | 32722241 | 7778110 | 6290 | GTGATGAAGAYGTCCCTGCTA | 0.291 | 0.288 | 1.489 | 0.965 | 0.526 |
| 22 | 7 | 32734764 | 2278819 | 6291 | AACTTTACTTKCAGAAGAGGG | 1.449 | 1.415 | 1.264 | 0.976 | 0.531 |
| 22 | 7 | 32745485 | 17469596 | 6292 | CAGGAGCTGARCAAACAACAT | 0.572 | 0.551 | 0.697 | 0.971 | 0.604 |
| 22 | 7 | 32816185 | 16881929 | 6293 | CTTCCACATCYGCTATTGAAG | 0.122 | 0.122 | 0.596 | 0.693 | 0.574 |
| 22 | 7 | 32829655 | 6942974 | 6294 | ATTAGAAAACSTTATTCAAAT | 0.000 | 0.000 | 0.141 | 0.257 | 0.619 |
| 22 | 7 | 32838422 | 17385980 | 6295 | CTTAATAAGGYTGTATCTTAA | 0.123 | 0.123 | 0.030 | 0.387 | 0.517 |
| 22 | 7 | 32847596 | 6462450 | 6296 | ATTTATTAAGYGTGACAAAAT | 0.379 | 0.377 | 0.062 | 0.125 | 0.406 |
| 22 | 7 | 32855971 | 7796184 | 6297 | ACTTGATAAGYGGCAGAAATG | 0.001 | 0.001 | 0.181 | 0.109 | 0.155 |
| 22 | 7 | 32862513 | 4723241 | 6298 | GCTTAGAAAGWCATCAAAATT | 0.293 | 0.279 | 0.206 | 0.100 | 0.164 |
| 22 | 7 | 32868656 | 10281012 | 6299 | TCTCTTAGTCRAGTGCAACTA | 0.755 | 0.734 | 0.217 | 0.078 | 0.054 |
| 22 | 7 | 32870950 | 10256717 | 6300 | AGAGAAAAACYGAAAATTTGC | 0.243 | 0.248 | 0.218 | 0.085 | 0.029 |
| 22 | 7 | 32891598 | 2392216 | 6301 | GCCACCTGAGRCCCTCCCCAG | 0.394 | 0.419 | 0.186 | 0.084 | 0.065 |
| 22 | 7 | 32900239 | 17393789 | 6302 | GTATACTAAGYCAGCCATTTC | 0.077 | 0.072 | 0.063 | 0.122 | 0.055 |
| 22 | 7 | 32906447 | 11981778 | 6303 | TTGTATTCCAMGGCCAAGAAT | 0.153 | 0.153 | 0.094 | 0.087 | 0.094 |
| 22 | 7 | 32914544 | 6964595 | 6304 | TACTAATCCCRGATCTGTTCC | 0.196 | 0.175 | 0.073 | 0.075 | 0.082 |
| 22 | 7 | 32944350 | 7796922 | 6305 | TTTGAACACTRCATGCATTTG | 0.442 | 0.440 | 0.053 | 0.072 | 0.086 |
| 22 | 7 | 32954589 | 17169808 | 6306 | CCCAAAGAAAYTGCCTTTTAT | 0.247 | 0.262 | 0.163 | 0.107 | 0.087 |
| 22 | 7 | 32961789 | 17169847 | 6307 | AAGTTTTCATRCTCTTTAGCA | 0.057 | 0.056 | 0.198 | 0.121 | 0.060 |
| 22 | 7 | 32969056 | 17169913 | 6308 | TGGGTACAAGYATAGTGTGCG | 0.686 | 0.687 | 0.228 | 0.102 | 0.058 |
| 22 | 7 | 33000206 | 759396 | 6309 | TCAGTTACCCSAAATTTACCT | 0.235 | 0.220 | 0.224 | 0.110 | 0.074 |
| 22 | 7 | 33011440 | 10486521 | 6310 | TTCCTCAACARTCTGTCTGAA | 0.627 | 0.583 | 0.220 | 0.101 | 0.094 |
| 22 | 7 | 33017621 | 6979640 | 6311 | AGGAAAAGAGSTGTATTGGGA | 0.202 | 0.201 | 0.085 | 0.094 | 0.100 |
| 22 | 7 | 33033399 | 1894872 | 6312 | GTGTGCATAARCATAAAGGCT | 0.039 | 0.039 | 0.138 | 0.196 | 0.222 |
| 22 | 7 | 33051018 | 17170131 | 6313 | GCCAAGGTTCRATATACATGT | 0.185 | 0.198 | 0.062 | 0.128 | 0.298 |
| 22 | 7 | 33057213 | 1420155 | 6314 | GGAATCACTGYTTTACTGAGT | 0.428 | 0.442 | 0.159 | 0.137 | 0.253 |
| 22 | 7 | 33063498 | 6952244 | 6315 | GATCAATCTCYACATATTTCT | 0.208 | 0.216 | 0.258 | 0.253 | 0.268 |
| 22 | 7 | 33069447 | 17170142 | 6316 | TTTCTATGCARTTTGATTTCT | 0.624 | 0.605 | 0.239 | 0.426 | 0.199 |
| 22 | 7 | 33076014 | 12671191 | 6317 | CTATGTCTTGSAAAATTTAAA | 0.355 | 0.357 | 0.495 | 0.418 | 0.233 |
| 22 | 7 | 33081762 | — | 6318 | TACAATATAAKGAGAAATATA | 0.135 | 0.143 | 0.801 | 0.393 | 0.166 |
| 22 | 7 | 33082095 | 7783612 | 6319 | TGTGACTTTTSATGATCTTTT | 1.170 | 1.160 | 0.555 | 0.356 | 0.274 |
| 22 | 7 | 33095157 | 9791453 | 6320 | TCAACCCTGAYATTTTTCTCT | 0.959 | 0.914 | 0.467 | 0.406 | 0.346 |
| 22 | 7 | 33102971 | 17170169 | 6321 | TAGGATTGAAWGTCGCAATAA | 0.008 | 0.008 | 0.495 | 0.272 | 0.341 |
| 22 | 7 | 33111593 | 17476738 | 6322 | AACAGTTCAARTCAGAGGGAG | 0.145 | 0.144 | 0.228 | 0.432 | 0.296 |
| 22 | 7 | 33124869 | 10273634 | 6323 | TTTTGTAATAYAAGGTTTCTT | 0.259 | 0.259 | 0.021 | 0.534 | 0.305 |
| 22 | 7 | 33141753 | 11771086 | 6324 | AATCCCACAARTATTTAATTT | 0.000 | 0.000 | 0.246 | 0.221 | 0.227 |
| 22 | 7 | 33147769 | 17170193 | 6325 | ACAGGGTTGCRTGTCACTGAC | 0.147 | 0.144 | 0.367 | 0.072 | 0.197 |
| 22 | 7 | 33154814 | 10230133 | 6326 | TGTAGAAATCWGTGTAGAAAT | 0.884 | 0.921 | 0.351 | 0.129 | 0.180 |
| 22 | 7 | 33166726 | 10951387 | 6327 | ATACTTGTCARATTAGAATTT | 0.000 | 0.000 | 0.255 | 0.143 | 0.089 |
| 22 | 7 | 33172356 | 17170203 | 6328 | ACTCACTTTASGAATGGGACA | 0.235 | 0.234 | 0.345 | 0.128 | 0.127 |
| 22 | 7 | 33180964 | 3735420 | 6329 | ATATGTAACTSATATATTCAC | 0.185 | 0.181 | 0.103 | 0.077 | 0.267 |
| 22 | 7 | 33184507 | 2109525 | 6330 | ACAGGAGGGAKCGTATGTACA | 0.319 | 0.311 | 0.066 | 0.136 | 0.343 |
| 22 | 7 | 33197001 | 17724206 | 6331 | TGAGCTTATCRGAGGCCATTT | 0.239 | 0.238 | 0.035 | 0.214 | 0.343 |
| 22 | 7 | 33206555 | 1419898 | 6332 | GCTCAAGTGCWTCTTTTTGGG | 0.177 | 0.176 | 0.080 | 0.324 | 0.276 |
| 22 | 7 | 33214153 | 6979138 | 6333 | TTCCCACTTTYGTGATTCAAG | 0.050 | 0.049 | 0.351 | 0.400 | 0.342 |
| 22 | 7 | 33224797 | 1419928 | 6334 | TACTCCTTATKGTAGTCATTT | 0.510 | 0.512 | 0.618 | 0.418 | 0.241 |
| 22 | 7 | 33232397 | 17170225 | 6335 | ATAAAATGTCRTGGAACCAGA | 1.212 | 1.190 | 0.802 | 0.350 | 0.549 |
| 22 | 7 | 33239234 | 9639675 | 6336 | TAACTTAATGMTTTTAAAGAG | 0.890 | 0.835 | 0.908 | 0.409 | 0.544 |
| 22 | 7 | 33245933 | 17170234 | 6337 | GTACCACATGYTAGGAGGCAC | 0.575 | 0.545 | 0.700 | 0.466 | 0.650 |
| 22 | 7 | 33251798 | 758779 | 6338 | GTTTATCATAWTTGGTAGGTA | 0.245 | 0.245 | 0.366 | 1.114 | 0.755 |
| 22 | 7 | 33257747 | 1860601 | 6339 | AGTTGAAAATKTTGCGAAAGC | 0.081 | 0.078 | 0.177 | 0.990 | 0.784 |
| 22 | 7 | 33271222 | 2392234 | 6340 | TGTTCATTTGWTCCATAGATT | 0.470 | 0.434 | 0.803 | 0.832 | 0.799 |
| 22 | 7 | 33276768 | 9886325 | 6341 | AATATTTTTCYGGATGTATCC | 0.322 | 0.349 | 0.766 | 0.811 | 0.768 |
| 22 | 7 | 33283598 | 4582453 | 6342 | TCACACACAARTTCACAAAGC | 1.954 | 2.124 | 1.095 | 0.740 | 0.958 |
| 22 | 7 | 33290160 | 11980608 | 6343 | GGCAGTCACTWTGAAGAACTA | 0.272 | 0.263 | 1.233 | 0.729 | 0.813 |
| 22 | 7 | 33297086 | 4723289 | 6344 | TCAGGAAAATMTACAAAAAAG | 0.722 | 0.712 | 1.145 | 0.708 | 0.668 |
| 22 | 7 | 33303288 | 6964756 | 6345 | TGTCATGTCCYTTTTTGTTTA | 0.790 | 0.791 | 0.457 | 1.047 | 0.554 |
| 22 | 7 | 33310906 | 11769528 | 6346 | GCTTTTACCGMGTATTGTTTT | 0.407 | 0.385 | 0.372 | 1.052 | 0.842 |
| 22 | 7 | 33319060 | 2392242 | 6347 | TTTTCAGGAAWGGATTATTTA | 0.244 | 0.255 | 0.661 | 0.500 | 0.954 |
| 22 | 7 | 33327456 | 17794801 | 6348 | ATGATTTACTRTAACATAAAA | 0.029 | 0.027 | 0.510 | 0.437 | 0.882 |
| 22 | 7 | 33342965 | 6462483 | 6349 | TTGTATTTTTRTTTAACAAAA | 1.449 | 1.398 | 0.409 | 0.703 | 0.893 |
| 22 | 7 | 33353932 | 6944252 | 6350 | TTCCATCTTCSTGCTCCCCTG | 0.447 | 0.441 | 0.315 | 0.546 | 0.420 |
| 22 | 7 | 33359477 | 2598408 | 6351 | TTTACTGATAYTGAAATACTC | 0.151 | 0.147 | 1.001 | 0.435 | 0.516 |
| 22 | 7 | 33367994 | 13226813 | 6352 | ATATAAACACWGTCTCAAATG | 0.021 | 0.022 | 0.482 | 0.492 | 0.392 |
| 22 | 7 | 33374179 | 1451018 | 6353 | TGTCTTATTTYCTACTTCTTC | 1.566 | 1.532 | 0.321 | 0.516 | 0.325 |
| 22 | 7 | 33380156 | 2252989 | 6354 | TGTGCATATTSCATATATCTC | 0.321 | 0.313 | 0.467 | 0.332 | 0.276 |
| 22 | 7 | 33391562 | 2598395 | 6355 | TATGCTTTTAYGTTGCATTGC | 0.048 | 0.048 | 0.493 | 0.264 | 0.284 |
| 22 | 7 | 33400828 | 1817055 | 6356 | ATGGATCCTTYTTCATTCGTT | 0.474 | 0.458 | 0.213 | 0.332 | 0.601 |
| 22 | 7 | 33409604 | 17170308 | 6357 | GTGTAGAAGTSCATACACAGA | 0.073 | 0.072 | 0.159 | 0.348 | 0.348 |
| 22 | 7 | 33414977 | 1451021 | 6358 | AAGGGAATATRTGAGGCCATG | 0.699 | 0.682 | 0.256 | 0.112 | 0.444 |
| 22 | 7 | 33423634 | 7791607 | 6359 | TGGGCTCAAAWGTAAGTACAC | 0.106 | 0.103 | 0.122 | 0.370 | 0.509 |
| 22 | 7 | 33440554 | 10486541 | 6360 | CCACAAAGCCRATAGTGATCA | 0.439 | 0.430 | 0.183 | 0.381 | 0.510 |
| 22 | 7 | 33446505 | 17170316 | 6361 | ACGATCTCTGKAATAGCTTCC | 0.100 | 0.099 | 0.535 | 0.496 | 0.279 |
| 22 | 7 | 33452874 | 1451011 | 6362 | AGAGATAGGCRAGGGTAAGGT | 0.287 | 0.277 | 0.540 | 0.629 | 1.407 |
| 22 | 7 | 33459609 | 16879181 | 6363 | GAGAAATCTTRCCATGTGGTG | 1.724 | 1.663 | 0.780 | 0.417 | 1.465 |
| 22 | 7 | 33466478 | 7811071 | 6364 | CAGCATTTGTRAGGCCAGGTG | 0.087 | 0.088 | 1.071 | 0.497 | 1.778 |
| 22 | 7 | 33478731 | 1451010 | 6365 | GGTTTTGTCAYCTGCTGGAAA | 0.949 | 0.998 | 0.929 | 2.997 | 1.890 |
| 22 | 7 | 33484293 | 4723309 | 6366 | TATCAGTTTTRTAAGTAAGCA | 0.000 | 0.000 | 0.254 | 3.046 | 1.808 |
| 22 | 7 | 33491746 | 6966497 | 6367 | CTTGTGGTGAYCAGCACTACT | 0.049 | 0.047 | 3.852 | 3.474 | 1.969 |
| 22 | 7 | 33497620 | 17170353 | 6368 | TACAACTTTCYTGCATGGAGA | 0.366 | 0.349 | 2.886 | 2.207 | 1.958 |
| 22 | 7 | 33505602 | 1376350 | 6369 | AACTGAGAGCRATTGTGGCAA | 5.035 | 4.267 | 2.738 | 2.352 | 1.809 |
| 22 | 7 | 33506023 | 990080 | 6370 | ATGCATCCACWACTTGATTCC | 0.286 | 0.288 | 2.775 | 1.879 | 2.037 |
| 22 | 7 | 33529577 | 7800309 | 6371 | ACCCTTGGAARGATAAAAAGC | 0.908 | 0.905 | 2.823 | 1.707 | 1.427 |
| 22 | 7 | 33540418 | 2232121 | 6372 | CAGATGTTGTRTGTTCTGGAA | 0.121 | 0.122 | 0.177 | 1.631 | 1.454 |
| 22 | 7 | 33549587 | 2042095 | 6373 | GGTTTTACAGRTAATGAGACA | 0.208 | 0.199 | 0.293 | 1.891 | 1.239 |
| 22 | 7 | 33557584 | 16879226 | 6374 | TATATACCCTRGTGACTGGTA | 0.131 | 0.131 | 0.071 | 0.226 | 1.130 |
| 22 | 7 | 33561770 | 2893475 | 6375 | AAAATCTATTYGAGAGCCCAG | 0.641 | 0.622 | 0.259 | 0.230 | 1.171 |
| 22 | 7 | 33572120 | 4723328 | 6376 | CTCCTTCCACYGCCACAGGAA | 0.030 | 0.030 | 0.244 | 0.135 | 1.164 |
| 22 | 7 | 33581291 | 12536852 | 6377 | TATTTTTGTGYATTTAATTTT | 0.821 | 0.912 | 0.332 | 0.144 | 0.170 |
| 22 | 7 | 33587768 | 10951394 | 6378 | CAGTCTTTCCYTCATACTCTT | 0.225 | 0.229 | 0.217 | 0.112 | 0.157 |
| 22 | 7 | 33593267 | — | 6379 | CTATCCTGGASAGCCTCCCTC | 0.328 | 0.314 | 0.274 | 0.158 | 0.098 |
| 22 | 7 | 33600653 | 10486618 | 6380 | GTGTACCCTTSTTTTCTATGT | 0.350 | 0.350 | 0.069 | 0.179 | 0.283 |
| 22 | 7 | 33607562 | 1362928 | 6381 | TTTTTGGGTAYAGTTCCCAGG | 0.220 | 0.220 | 0.098 | 0.217 | 0.315 |
| 22 | 7 | 33613722 | 17170440 | 6382 | ATCAAATCGTMAAAAAAAAAG | 0.081 | 0.082 | 0.210 | 0.120 | 0.393 |
| 22 | 7 | 33621606 | 7794034 | 6383 | TAAAAGAAACRTGAAACCCCA | 0.376 | 0.392 | 0.184 | 0.418 | 0.554 |
| 22 | 7 | 33627530 | 17740751 | 6384 | GCTAATGAGGSCGAGTCATGT | 0.777 | 0.740 | 0.217 | 0.446 | 0.556 |
| 22 | 7 | 33638981 | 7785398 | 6385 | ATTTGGCCCCYATTAGTAACA | 0.224 | 0.240 | 0.843 | 0.481 | 0.423 |
| 22 | 7 | 33645497 | 17740905 | 6386 | GACAGCCTGGRCACTGGAGAG | 0.315 | 0.313 | 0.853 | 0.826 | 0.533 |
| 22 | 7 | 33652339 | — | 6387 | ATAACAGTGTSTGACAAATGG | 1.550 | 1.472 | 0.663 | 0.814 | 0.474 |
| 22 | 7 | 33661735 | 10243554 | 6388 | GCTGGTTCAGRCATTTTCTCT | 0.365 | 0.356 | 1.120 | 0.775 | 0.589 |
| 23 | 7 | 127239803 | 17151653 | 6389 | TTATTTGAGGRGCTCCCCACC | 0.252 | 0.252 | 0.112 | 0.071 | 0.173 |
| 23 | 7 | 127254714 | 6467157 | 6390 | TCGGAGTGGAYGTGGACTTAC | 0.554 | 0.538 | 0.133 | 0.084 | 0.416 |
| 23 | 7 | 127262797 | — | 6391 | CTGTCCAGTGKGCCCCATGTG | 0.239 | 0.225 | 0.167 | 0.191 | 0.310 |
| 23 | 7 | 127282576 | 6962635 | 6392 | GGAAAAACACRCAGCCTGTTT | 0.173 | 0.174 | 0.248 | 0.266 | 0.320 |
| 23 | 7 | 127289004 | 6966724 | 6393 | AATAGGATGGRTATCTGCCCG | 0.359 | 0.347 | 0.321 | 0.753 | 0.240 |
| 23 | 7 | 127296399 | 17151707 | 6394 | GAGGCGTGAGRACAAAAGGAA | 0.570 | 0.526 | 0.424 | 0.705 | 0.315 |
| 23 | 7 | 127300637 | 3757754 | 6395 | GCATTGTTAAYGTTCATCATC | 0.780 | 0.778 | 1.294 | 0.600 | 0.318 |
| 23 | 7 | 127317264 | 124756 | 6396 | TTGGCAGGAGRCAAAGTCCTG | 0.000 | 0.000 | 1.099 | 0.553 | 0.283 |
| 23 | 7 | 127325849 | — | 6397 | GATCACAGAAYACCTGAATGC | 1.966 | 1.896 | 1.001 | 0.673 | 0.390 |
| 23 | 7 | 127333672 | 6954161 | 6398 | CCTCAGAGATKTCACAGTAGA | 0.010 | 0.010 | 0.615 | 0.586 | 0.276 |
| 23 | 7 | 127360351 | 10258271 | 6399 | TATCTTCTTGSACCCAATATA | 0.208 | 0.199 | 0.616 | 0.414 | 0.256 |
| 23 | 7 | 127377349 | 322740 | 6400 | ACAGCATTAGKTTTGAAAAAT | 0.081 | 0.079 | 0.033 | 0.401 | 0.321 |
| 23 | 7 | 127383147 | 53125 | 6401 | CTGAAACGCASTTTTTAAGAT | 0.557 | 0.516 | 0.032 | 0.289 | 0.285 |
| 23 | 7 | 127389755 | 10447854 | 6402 | ATATAATTGCYGGGGACATAC | 0.078 | 0.081 | 0.144 | 0.016 | 1.220 |
| 23 | 7 | 127396223 | 10954170 | 6403 | AATAAATCACMAAGTATGAGT | 0.037 | 0.037 | 0.132 | 0.049 | 1.073 |
| 23 | 7 | 127408308 | 17293437 | 6404 | ACAAAAAATARTAACAGCAGC | 0.753 | 0.742 | 0.043 | 0.047 | 1.047 |
| 23 | 7 | 127423722 | 896178 | 6405 | GGCCCAGACGMTGCCATCATT | 0.070 | 0.066 | 0.120 | 1.228 | 0.636 |
| 23 | 7 | 127435720 | — | 6406 | TGTTTATTACKTTGGACCGCA | 0.070 | 0.070 | 0.155 | 1.065 | 0.682 |
| 23 | 7 | 127442525 | 2060736 | 6407 | TAACCGCGCCSTTCATTCTGT | 0.523 | 0.510 | 1.803 | 1.211 | 0.624 |
| 23 | 7 | 127451999 | 11764840 | 6408 | CTTAGTTGCAWTCCGATATGC | 0.160 | 0.140 | 2.105 | 1.399 | 0.707 |
| 23 | 7 | 127464579 | 1376349 | 6409 | TGGTTTAAGCYTTTGAGCTCT | 4.300 | 4.364 | 2.069 | 1.113 | 0.737 |
| 23 | 7 | 127476021 | 4731426 | 6410 | TGCTAAATACSCGCTGTTGGC | 0.114 | 0.113 | 1.976 | 1.183 | 0.862 |
| 23 | 7 | 127484013 | 11763517 | 6411 | GTGGCCATTAYTTGAGAGTGA | 0.469 | 0.465 | 1.753 | 1.197 | 0.976 |
| 23 | 7 | 127493080 | 2060715 | 6412 | GCTAAGTGACRGATACATGGG | 0.442 | 0.460 | 0.093 | 1.284 | 0.965 |
| 23 | 7 | 127499508 | 10954175 | 6413 | CCAGGTGAAARGCCCAGCGCT | 0.199 | 0.203 | 0.153 | 1.353 | 0.948 |
| 23 | 7 | 127507182 | 10274395 | 6414 | CTACCCCATTSTGCAGTGAAA | 0.000 | 0.000 | 0.189 | 0.233 | 0.924 |
| 23 | 7 | 127517729 | 7811892 | 6415 | GGACTTGCTTYAGCCTTACAC | 0.345 | 0.353 | 0.239 | 0.357 | 0.912 |
| 23 | 7 | 127523017 | 12534540 | 6416 | CATGTTAAAARTTCAGAGGAA | 0.642 | 0.601 | 0.351 | 0.292 | 1.042 |
| 23 | 7 | 127542555 | 12538391 | 6417 | CCAAGTCATGYCTCGAGGTGA | 0.601 | 0.566 | 0.587 | 0.199 | 0.215 |
| 23 | 7 | 127550209 | 4731441 | 6418 | AGAGATGCATWCACAAATAAA | 0.461 | 0.481 | 0.510 | 0.252 | 0.239 |
| 23 | 7 | 127556786 | 12536858 | 6419 | CTAGGTGTTCRCTACCACAAA | 0.710 | 0.707 | 0.299 | 0.417 | 0.176 |
| 23 | 7 | 127567084 | 359650 | 6420 | GAAATCTAAAYGAAAACGTGA | 0.133 | 0.133 | 0.240 | 0.401 | 0.162 |
| 23 | 7 | 127581730 | 962294 | 6421 | AAATCTAACTSTGTCTCTATG | 0.034 | 0.033 | 0.313 | 0.307 | 0.149 |
| 23 | 7 | 127618814 | 885343 | 6422 | CAGCGTAGAGYCAACCCTCCA | 0.429 | 0.442 | 0.181 | 0.175 | 0.171 |
| 23 | 7 | 127667809 | 17152065 | 6423 | CTTACTATGCRTCAGACACCA | 0.773 | 0.744 | 0.229 | 0.155 | 0.173 |
| 23 | 7 | 127678648 | 13222212 | 6424 | ATTTTGGGTARAATCCCCTTT | 0.306 | 0.312 | 0.245 | 0.067 | 0.135 |
| 23 | 7 | 127684249 | 17152085 | 6425 | CTAAAAATAGYTTTGAGTAGT | 0.298 | 0.312 | 0.225 | 0.075 | 0.138 |
| 23 | 7 | 127704972 | — | 6426 | GGATGGCAAARTGTTTTATTT | 0.049 | 0.044 | 0.059 | 0.129 | 0.158 |
| 23 | 7 | 127721306 | 2562737 | 6427 | TCACTCAAATWATGAGCCCAA | 0.397 | 0.396 | 0.048 | 0.110 | 0.178 |
| 24 | 7 | 145001785 | 6943520 | 6428 | TTTATAATGAYGTTTTTACCA | 0.556 | 0.555 | 0.384 | 0.307 | 0.172 |
| 24 | 7 | 145018059 | 4601231 | 6429 | TCTTTTCTGAKACTACGGAAG | 0.639 | 0.617 | 0.519 | 0.228 | 0.139 |
| 24 | 7 | 145034566 | 10321215 | 6430 | CTCTCTCTTAMGCTCTAATGA | 0.118 | 0.119 | 0.317 | 0.212 | 0.283 |
| 24 | 7 | 145046677 | 4392813 | 6431 | ATCTCCCACTRTCTCCATTTG | 0.517 | 0.514 | 0.157 | 0.231 | 0.264 |
| 24 | 7 | 145055277 | 7780753 | 6432 | CCCAGGTCCARTTGGGGAAAT | 0.134 | 0.144 | 0.070 | 0.129 | 0.250 |
| 24 | 7 | 145071042 | 4355696 | 6433 | AAAATGTTAARGGGAATAAAA | 0.068 | 0.065 | 0.102 | 0.299 | 0.268 |
| 24 | 7 | 145083737 | 7799145 | 6434 | TATAGTTATCYGCAACTTACT | 0.333 | 0.342 | 0.044 | 0.186 | 0.261 |
| 24 | 7 | 145096619 | 7786054 | 6435 | GTAATACGTGYGTAGTGGTAT | 0.251 | 0.250 | 0.384 | 0.223 | 0.184 |
| 24 | 7 | 145114530 | 7777578 | 6436 | AGTGAGATTTKGTAGTGTTTC | 0.176 | 0.175 | 0.399 | 0.212 | 0.143 |
| 24 | 7 | 145126258 | — | 6437 | CCATTCCAAGSAAACTCCATT | 1.397 | 1.355 | 0.369 | 0.199 | 0.094 |
| 24 | 7 | 145137729 | 6968866 | 6438 | ACCACAAGTCYGATAAGGCTT | 0.069 | 0.070 | 0.473 | 0.240 | 0.119 |
| 24 | 7 | 145139853 | 10226110 | 6439 | GGTGGCCACAYTGTACTAGAC | 0.274 | 0.265 | 0.440 | 0.207 | 0.090 |
| 24 | 7 | 145155154 | 17169920 | 6440 | TGAAGTTTTAYACACAGTTCT | 0.482 | 0.496 | 0.102 | 0.192 | 0.096 |
| 24 | 7 | 145164360 | 17169926 | 6441 | GTCACTCAGGKTTATTACGCT | 0.144 | 0.142 | 0.115 | 0.244 | 0.093 |
| 24 | 7 | 145168812 | 4726779 | 6442 | ATAGCCAAGCYTCATTTTCCT | 0.270 | 0.275 | 0.104 | 0.054 | 0.066 |
| 24 | 7 | 145205736 | 10277243 | 6443 | TGCTTGCGTTRTTATCAGTGT | 0.208 | 0.210 | 0.064 | 0.068 | 0.110 |
| 24 | 7 | 145211439 | 2204466 | 6444 | CACAAGGAATRGCAAGAAGAG | 0.240 | 0.232 | 0.103 | 0.049 | 0.223 |
| 24 | 7 | 145222414 | 4726781 | 6445 | TTTTACTGCAWTATTAACTAT | 0.000 | 0.000 | 0.111 | 0.014 | 0.062 |
| 24 | 7 | 145231727 | 4726782 | 6446 | GATGACTTACYTAACAGATAT | 0.328 | 0.318 | 0.078 | 0.065 | 0.080 |
| 24 | 7 | 145241950 | 6464728 | 6447 | AAAGCACATTYGTAGATTATA | 0.280 | 0.276 | 0.046 | 0.194 | 0.300 |
| 24 | 7 | 145245095 | 2692359 | 6448 | CCATAATATTYCTCTAGTTCC | 0.012 | 0.011 | 0.146 | 0.173 | 0.288 |
| 24 | 7 | 145261967 | 1260126 | 6449 | ATCTATGCTGWTTTAGAGGAA | 0.001 | 0.001 | 0.340 | 0.209 | 0.770 |
| 24 | 7 | 145291239 | 7807672 | 6450 | TACAACTTTCRCCTATGCTAA | 0.681 | 0.700 | 0.240 | 0.533 | 0.785 |
| 24 | 7 | 145307229 | 1405115 | 6451 | AATTTTATTGWATTTATTCTC | 1.030 | 1.017 | 0.358 | 0.547 | 0.841 |
| 24 | 7 | 145319649 | 4612252 | 6452 | ATTATTTTCTRTTTTACAAGT | 0.038 | 0.039 | 1.133 | 1.141 | 0.894 |
| 24 | 7 | 145332263 | 7795190 | 6453 | TTTTTGTTCCMTGATACTATT | 0.367 | 0.368 | 0.964 | 1.277 | 0.780 |
| 24 | 7 | 145345572 | 6464738 | 6454 | AGACAAGACTRCTTCATTTTT | 1.770 | 1.730 | 1.512 | 1.435 | 0.835 |
| 24 | 7 | 145351794 | 802539 | 6455 | ATTTTTAGGARCTTTCATTTT | 0.395 | 0.384 | 1.882 | 1.534 | 0.814 |
| 24 | 7 | 145359146 | 2693307 | 6456 | AGCAAGTTACRTTTTTATCAA | 2.407 | 2.405 | 2.323 | 1.184 | 0.857 |
| 24 | 7 | 145366077 | 1526079 | 6457 | CGCAGGAGTGMACCTGTTTTC | 0.338 | 0.345 | 1.453 | 1.518 | 0.890 |
| 24 | 7 | 145372744 | 17170000 | 6458 | GTTCCAAAAGSAAGCTTATGT | 0.000 | 0.000 | 1.235 | 1.365 | 0.748 |
| 24 | 7 | 145385679 | 802522 | 6459 | AGTTTAGTGARCTCAAGGTGA | 0.000 | 0.000 | 0.115 | 0.632 | 0.657 |
| 24 | 7 | 145390594 | 2430327 | 6460 | TCCTGGAATTMATCATGAGAC | 0.041 | 0.041 | 0.012 | 0.599 | 0.887 |
| 24 | 7 | 145411268 | 6946638 | 6461 | AAGCACAATTYGAAGCACTGG | 0.000 | 0.000 | 0.012 | 0.008 | 0.903 |
| 24 | 7 | 145419484 | — | 6462 | TCAAAGCATGYAGTGAAGGGA | 0.077 | 0.072 | 0.015 | 0.042 | 0.677 |
| 24 | 7 | 145431326 | 10262823 | 6463 | TCATGATGTTRAGGGAAATAT | 0.157 | 0.159 | 0.014 | 0.145 | 0.730 |
| 24 | 7 | 145450428 | 7795585 | 6464 | ATACATTTGTWTTTTTTCAAT | 0.243 | 0.251 | 0.080 | 0.139 | 0.381 |
| 24 | 7 | 145460671 | 17170038 | 6465 | CACAAACTGARCTTACTTTAC | 0.059 | 0.057 | 0.342 | 0.368 | 0.377 |
| 24 | 7 | 145476714 | 10248899 | 6466 | AGCCTTCATTMTTAATGTTTG | 0.707 | 0.705 | 0.405 | 0.388 | 0.474 |
| 24 | 7 | 145489102 | 17170058 | 6467 | AGTCTAATTAYTATTGTAGAC | 0.915 | 0.923 | 0.696 | 0.630 | 0.474 |
| 24 | 7 | 145500557 | 12703803 | 6468 | GTCTATTAACKTAATAAGCCA | 0.351 | 0.364 | 0.892 | 0.689 | 0.536 |
| 24 | 7 | 145512007 | 17170073 | 6469 | GGAGGATTTTYGCCTATAAAA | 0.959 | 0.940 | 1.007 | 0.868 | 0.632 |
| 24 | 7 | 145520605 | 10238194 | 6470 | TTGTATTACARCTGTTTTAAA | 0.534 | 0.529 | 0.787 | 1.048 | 0.801 |
| 24 | 7 | 145528627 | 13247443 | 6471 | GTAGCCTCCAWATTTTGTTCA | 0.893 | 0.940 | 1.049 | 0.912 | 0.930 |
| 24 | 7 | 145535097 | 1358079 | 6472 | TGTTCTCGACYTTGTTTCTGC | 0.338 | 0.339 | 0.872 | 0.892 | 0.977 |
| 24 | 7 | 145541459 | 802194 | 6473 | TCTGGGGACTRCCTTGAGTCA | 0.879 | 0.875 | 0.737 | 1.082 | 1.096 |
| 24 | 7 | 145553353 | 1724513 | 6474 | TTATTAACTAYTCAAATACTC | 0.000 | 0.000 | 0.742 | 0.953 | 1.123 |
| 24 | 7 | 145557979 | 1718093 | 6475 | ATTGAACTTTSTGTCTTTTAA | 0.304 | 0.327 | 0.949 | 0.956 | 0.987 |
| 24 | 7 | 145568439 | 1724497 | 6476 | ATGGTCCCTCRTCACGCTGTG | 0.913 | 0.981 | 0.845 | 0.911 | 0.954 |
| 24 | 7 | 145579861 | 17505462 | 6477 | GTCACCAAAAYTTTTATCCAG | 0.792 | 0.827 | 0.860 | 1.071 | 0.766 |
| 24 | 7 | 145584471 | 1639470 | 6478 | GCATATTTCAYGAATGCCCCT | 0.721 | 0.767 | 1.011 | 0.898 | 0.764 |
| 24 | 7 | 145597926 | 7805539 | 6479 | ATACAAGAAGRGAACTGTTAT | 0.620 | 0.633 | 1.102 | 0.741 | 0.639 |
| 24 | 7 | 145606854 | 6968491 | 6480 | CAAATGCACCRTTACATAGGC | 0.642 | 0.661 | 0.775 | 0.666 | 0.603 |
| 24 | 7 | 145611435 | 17170116 | 6481 | TGATTTGGTTYAAATAAGACT | 0.951 | 1.005 | 0.466 | 0.572 | 0.421 |
| 24 | 7 | 145625916 | — | 6482 | AGTTTCCATGYACAACTAAAT | 0.183 | 0.177 | 0.276 | 0.437 | 0.369 |
| 24 | 7 | 145632277 | 10227262 | 6483 | CTACAGTGTAYTTCACCCAAA | 0.031 | 0.031 | 0.276 | 0.279 | 0.356 |
| 24 | 7 | 145656868 | 17584924 | 6484 | CGTCATTCTTMCTTAAAAGCA | 0.096 | 0.095 | 0.100 | 0.164 | 0.201 |
| 24 | 7 | 145664230 | 17170134 | 6485 | CAAGAACATTKATGATCCCTT | 0.597 | 0.604 | 0.079 | 0.093 | 0.152 |
| 24 | 7 | 145677193 | 2693406 | 6486 | GCAGAAACCAYCTAGACTTGT | 0.295 | 0.294 | 0.071 | 0.026 | 0.078 |
| 24 | 7 | 145684853 | 2693392 | 6487 | TTGGATTGATKAAACTTAGGG | 0.153 | 0.119 | 0.091 | 0.018 | 0.049 |
| 24 | 7 | 145691340 | 802013 | 6488 | AAATACTAGGRCAGTTAAGAA | 0.042 | 0.041 | 0.037 | 0.028 | 0.029 |
| 24 | 7 | 145697862 | 10249254 | 6489 | GTGAACFGATWAACTTTGCAG | 0.176 | 0.174 | 0.014 | 0.025 | 0.039 |
| 24 | 7 | 145704173 | 10249436 | 6490 | TCACATTTCAMACTTTATCTT | 0.291 | 0.285 | 0.020 | 0.006 | 0.042 |
| 24 | 7 | 145710348 | 10229049 | 6491 | AAACAGGAGTKGTGACTTTTC | 0.002 | 0.002 | 0.015 | 0.005 | 0.090 |
| 24 | 7 | 145717706 | 6960544 | 6492 | ATATATAATASTCATTAATAG | 0.000 | 0.000 | 0.009 | 0.084 | 0.090 |
| 24 | 7 | 145725199 | 7789059 | 6493 | ATCAAAGTCAMTAGAATATGA | 0.063 | 0.060 | 0.002 | 0.092 | 0.068 |
| 24 | 7 | 145734775 | 13247654 | 6494 | TCTAACATTTRTCTTTGAGAG | 0.117 | 0.113 | 0.352 | 0.189 | 0.074 |
| 24 | 7 | 145741781 | 347195 | 6495 | AATACTATTTRTCTTATTTAT | 0.000 | 0.000 | 0.262 | 0.161 | 0.155 |
| 24 | 7 | 145747802 | 17170192 | 6496 | CAAGCTAATTKTCTTCCTTAT | 1.064 | 1.073 | 0.494 | 0.261 | 0.181 |
| 24 | 7 | 145754722 | 347223 | 6497 | TGTAAACTTAYGTTCTCACAA | 0.121 | 0.115 | 0.492 | 0.261 | 0.188 |
| 24 | 7 | 145761651 | 6968860 | 6498 | ATTGGGTCATMTCTAATATAA | 0.732 | 0.724 | 0.492 | 0.453 | 0.215 |
| 24 | 7 | 145768739 | 13229953 | 6499 | GGAAGAGTACMCTATGGGCCA | 0.091 | 0.091 | 0.215 | 0.535 | 0.247 |
| 24 | 7 | 145775759 | 13235730 | 6500 | CCTTCCAATASTTTGCAATTG | 0.000 | 0.000 | 0.517 | 0.459 | 0.388 |
| 24 | 7 | 145781202 | 2215327 | 6501 | TATAGTAACARTAATGTATAA | 0.000 | 0.000 | 0.305 | 0.265 | 0.681 |
| 24 | 7 | 145790224 | 10277654 | 6502 | TGCCTGAAATYATGCCTTGTT | 0.729 | 0.726 | 0.390 | 0.273 | 0.700 |
| 24 | 7 | 145800130 | 2372761 | 6503 | CTGTTTTCAARTTATAATTTC | 0.345 | 0.365 | 0.360 | 0.408 | 0.714 |
| 24 | 7 | 145808805 | 1494459 | 6504 | GTCACCAAATSGTAGTTGTTT | 0.226 | 0.231 | 0.282 | 0.915 | 0.508 |
| 24 | 7 | 145826907 | 2642483 | 6505 | GTCTTCTACAYTTCCTTTTGT | 0.382 | 0.360 | 0.453 | 0.769 | 0.616 |
| 24 | 7 | 145839399 | 344453 | 6506 | TGTGAGGTCARTTGGTTAACC | 0.191 | 0.195 | 0.932 | 0.790 | 0.699 |
| 24 | 7 | 145845612 | 17170221 | 6507 | CATATATATCYGGTCTTGGCA | 1.219 | 1.199 | 0.919 | 0.600 | 0.716 |
| 24 | 7 | 145851145 | 344470 | 6508 | CTTGAGACCCYAAAACAGTAT | 1.543 | 1.548 | 0.979 | 0.636 | 0.885 |
| 24 | 7 | 145858219 | 344465 | 6509 | TAGACTTACTRAAATTATACC | 0.095 | 0.092 | 0.965 | 0.910 | 0.801 |
| 24 | 7 | 145865148 | 7811006 | 6510 | ATCCCATTTTRTAACATTGTA | 0.560 | 0.516 | 0.622 | 0.812 | 0.743 |
| 24 | 7 | 145879656 | — | 6511 | ATCCTCTAAGKAGAATATTTG | 0.151 | 0.190 | 0.379 | 1.134 | 0.724 |
| 24 | 7 | 145884415 | 6956550 | 6512 | CCCTGAATGARTGAAGCTATA | 0.490 | 0.468 | 0.463 | 0.783 | 0.757 |
| 24 | 7 | 145895515 | 10952659 | 6513 | TAGAACATTCSTCCAATAATG | 0.968 | 0.979 | 0.665 | 0.419 | 0.677 |
| 24 | 7 | 145902613 | 10952661 | 6514 | ATTAAACAAGYTGCCCCAATT | 0.262 | 0.258 | 0.700 | 0.525 | 0.706 |
| 24 | 7 | 145918045 | 11766778 | 6515 | AATATATTCTWTCTTTTAAAT | 0.708 | 1.157 | 0.750 | 0.485 | 0.432 |
| 24 | 7 | 145927493 | 1541512 | 6516 | AGTGTCCCCARCCATAAATTT | 0.274 | 0.257 | 0.422 | 0.470 | 0.149 |
| 24 | 7 | 145943250 | — | 6517 | AGGAATCACAYTTTTAAAAAT | 0.000 | 0.000 | 0.485 | 0.393 | 0.200 |
| 24 | 7 | 145962677 | 10234284 | 6518 | TTTTTATTGCSTTTATTTTTC | 0.340 | 0.340 | 0.113 | 0.155 | 0.350 |
| 24 | 7 | 145970176 | 10282480 | 6519 | GAACTGTATAYCTAACAGGTA | 0.368 | 0.366 | 0.106 | 0.103 | 0.628 |
| 24 | 7 | 145983299 | 7781516 | 6520 | CTAGGAAAGTSGTCTTATTCA | 0.050 | 0.042 | 0.071 | 0.038 | 0.569 |
| 24 | 7 | 145984741 | 6955151 | 6521 | ATACTGTTTTYCAGGTAGCTA | 0.262 | 0.264 | 0.026 | 0.222 | 0.383 |
| 24 | 7 | 145997122 | 4367451 | 6522 | GGATCATAACRTGCTATTTCC | 0.110 | 0.112 | 0.032 | 0.491 | 0.562 |
| 24 | 7 | 146008270 | 17170284 | 6523 | AACTGGAAGTSCATGCGATTC | 0.026 | 0.026 | 0.373 | 0.441 | 0.420 |
| 24 | 7 | 146015977 | 17170287 | 6524 | TGTATTGGTTKTCTCAAGAAT | 0.415 | 0.422 | 0.882 | 0.393 | 0.403 |
| 24 | 7 | 146022793 | 17483333 | 6525 | AGCCAATGAAWTAATCTATCA | 1.347 | 1.278 | 0.877 | 0.670 | 0.342 |
| 24 | 7 | 146029536 | 10216156 | 6526 | GCTACCATACRATCGATATCC | 1.466 | 1.411 | 1.036 | 0.756 | 0.296 |
| 24 | 7 | 146034961 | 10500168 | 6527 | TAGTGTTGTCYTGTAGCATAG | 0.177 | 0.178 | 1.204 | 0.718 | 0.344 |
| 24 | 7 | 146047361 | 11765622 | 6528 | ATCCCCTTTARACCTCAAGAA | 0.064 | 0.203 | 0.860 | 0.741 | 0.431 |
| 24 | 7 | 146059994 | 11773694 | 6529 | AATCGCACCTKTATTTCTGGC | 0.979 | 0.987 | 0.315 | 0.623 | 0.465 |
| 24 | 7 | 146074532 | 2049396 | 6530 | CTACAAAAAAWCTAAAGGAAT | 0.396 | 0.431 | 0.300 | 0.460 | 0.632 |
| 24 | 7 | 146083998 | — | 6531 | ATAAATGAAAYTGTCAAGATA | 0.135 | 0.135 | 0.273 | 0.284 | 0.865 |
| 24 | 7 | 146090580 | 13228798 | 6532 | GATAGGTTTGYCATCACAGAG | 0.116 | 0.114 | 0.134 | 0.330 | 0.868 |
| 24 | 7 | 146096763 | 12703865 | 6533 | CCAGTTACGCRGTGGTTACTT | 0.020 | 0.020 | 0.201 | 0.530 | 0.758 |
| 24 | 7 | 146112499 | 1524340 | 6534 | AACTGAAAACKGCAATTGTCA | 0.555 | 0.534 | 0.275 | 0.641 | 0.611 |
| 24 | 7 | 146119599 | 12703874 | 6535 | ATGCCGTCAGSTAAACATAAT | 0.522 | 0.521 | 0.601 | 0.646 | 0.601 |
| 24 | 7 | 146126667 | 13234249 | 6536 | ATCATAAATGYATTGTTACTG | 0.418 | 0.383 | 1.073 | 0.743 | 0.582 |
| 24 | 7 | 146139802 | 10281702 | 6537 | TTGGTCTCTCYGGAGGCAGGA | 1.230 | 1.198 | 1.163 | 0.876 | 0.445 |
| 24 | 7 | 146148505 | 17170304 | 6538 | CTATACTTCTSCAATACAATT | 1.566 | 1.522 | 1.301 | 0.936 | 0.439 |
| 24 | 7 | 146165900 | 1524347 | 6539 | TATTTAACATRTCACAAAATG | 0.000 | 0.000 | 1.508 | 0.833 | 0.406 |
| 24 | 7 | 146172529 | 982512 | 6540 | TAGATATATAMATGTTAGACG | 0.000 | 0.000 | 0.926 | 0.797 | 0.507 |
| 24 | 7 | 146178225 | 7812091 | 6541 | CTCTCACTAAYGTTGAAATGT | 0.000 | 0.000 | 0.056 | 0.792 | 0.655 |
| 24 | 7 | 146189573 | 10266622 | 6542 | TATCAGAATARTATCAGATCT | 0.089 | 0.087 | 0.064 | 0.332 | 0.755 |
| 24 | 7 | 146197132 | 6973868 | 6543 | TTAGAGCAGARTATGCATGCA | 0.180 | 0.175 | 0.093 | 0.094 | 0.646 |
| 24 | 7 | 146204462 | 6975159 | 6544 | CTCAAAGTTGYTTACTTACTA | 0.309 | 0.303 | 0.045 | 0.147 | 0.561 |
| 24 | 7 | 146215148 | 10952669 | 6545 | CAGGTGTATARCATGAACTTA | 0.423 | 0.413 | 0.156 | 0.246 | 0.397 |
| 24 | 7 | 146224977 | — | 6546 | TCAAATCAATYAGACAAATAC | 0.016 | 0.043 | 0.306 | 0.174 | 0.147 |
| 24 | 7 | 146230781 | 11973305 | 6547 | TATTGATTTASCATTAATCAT | 0.618 | 0.624 | 0.525 | 0.188 | 0.114 |
| 24 | 7 | 146238152 | 13238590 | 6548 | GTGTGTGAAAYTTGCAAAATA | 0.656 | 0.662 | 0.384 | 0.279 | 0.084 |
| 24 | 7 | 146252578 | 6958777 | 6549 | CACATTAAAGWTCTGATCTGA | 0.778 | 0.871 | 0.417 | 0.274 | 0.084 |
| 24 | 7 | 146269497 | 9640492 | 6550 | AAACAGTTAAKTCAGGATTCT | 0.106 | 0.102 | 0.409 | 0.213 | 0.087 |
| 24 | 7 | 146275727 | 6959187 | 6551 | TATGATGACARCTGTGATAAT | 0.112 | 0.112 | 0.283 | 0.232 | 0.078 |
| 24 | 7 | 146282531 | 17170336 | 6552 | CTACCTTTACWAACTTGCTCT | 0.642 | 0.600 | 0.089 | 0.184 | 0.079 |
| 24 | 7 | 146293204 | 1390718 | 6553 | GAACACATATYGACTGTATTT | 0.241 | 0.253 | 0.083 | 0.097 | 0.084 |
| 24 | 7 | 146302372 | 1826843 | 6554 | CAGCTGTCCTRTGATATCAGC | 0.027 | 0.027 | 0.122 | 0.024 | 0.102 |
| 24 | 7 | 146308562 | 4725706 | 6555 | ACTCTGCACAYGTTGCTGCTA | 0.024 | 0.024 | 0.024 | 0.038 | 0.089 |
| 24 | 7 | 146313964 | 1496543 | 6556 | TAACAGCAAARGGTTCTTCAA | 0.374 | 0.363 | 0.010 | 0.057 | 0.049 |
| 24 | 7 | 146320950 | 7801686 | 6557 | TTAATGCTTAYACTAAATTCT | 0.072 | 0.072 | 0.035 | 0.024 | 0.011 |
| 24 | 7 | 146327696 | 1018073 | 6558 | GTGAACAGAAYTGTATTTTTG | 0.066 | 0.063 | 0.080 | 0.036 | 0.013 |
| 24 | 7 | 146373578 | 1390723 | 6559 | CAATTGCTATRAAAGCATCAT | 0.309 | 0.287 | 0.056 | 0.033 | 0.050 |
| 24 | 7 | 146384563 | — | 6560 | TCCGTGCTCTKTGCTGGAGGC | 0.395 | 0.383 | 0.132 | 0.021 | 0.031 |
| 24 | 7 | 146392045 | 9769600 | 6561 | ACAGAAGTCTRTTGAACAGGA | 0.217 | 0.222 | 0.134 | 0.016 | 0.033 |
| 24 | 7 | 146399127 | 17170379 | 6562 | GTTTTTAGACYCTCCTCTGTG | 0.477 | 0.456 | 0.067 | 0.100 | 0.042 |
| 24 | 7 | 146405943 | 10279700 | 6563 | GCTAAGTAAGYTGATATTCCT | 0.089 | 0.090 | 0.044 | 0.149 | 0.035 |
| 24 | 7 | 146413128 | 10268597 | 6564 | AGTTGAGGACMATGTTTCAAA | 0.028 | 0.024 | 0.231 | 0.161 | 0.025 |
| 24 | 7 | 146435941 | 2620440 | 6565 | TCTTAAGTAAYGAAAAGATTA | 0.174 | 0.176 | 0.212 | 0.146 | 0.036 |
| 24 | 7 | 146448938 | 4269450 | 6566 | ATCTAATGGGRAAATTTGAAG | 1.071 | 1.148 | 0.266 | 0.102 | 0.059 |
| 24 | 7 | 146456215 | 2692146 | 6567 | AGCTTCTGAAWTAGGGATTTT | 0.000 | 0.000 | 0.389 | 0.060 | 0.079 |
| 24 | 7 | 146463556 | 16883333 | 6568 | ACTCAGCTCTSTTGGTGCAGC | 0.301 | 0.311 | 0.330 | 0.074 | 0.109 |
| 24 | 7 | 146470362 | 747139 | 6569 | AAACAGAGACKGAGGAAATCT | 0.232 | 0.230 | 0.041 | 0.160 | 0.081 |
| 24 | 7 | 146477116 | 16883356 | 6570 | CAGCTATAAAMACATTTGCTT | 0.051 | 0.052 | 0.037 | 0.215 | 0.068 |
| 24 | 7 | 146487462 | 10254249 | 6571 | CTGCTCTATTKCCTGTTACTA | 0.091 | 0.092 | 0.063 | 0.136 | 0.156 |
| 24 | 7 | 146496034 | 826825 | 6572 | GATTTTCTGAYAACAATGAAT | 0.232 | 0.234 | 0.116 | 0.083 | 0.167 |
| 24 | 7 | 146503811 | 13233234 | 6573 | GGTCTATACAWCGGAAAAAAG | 0.553 | 0.506 | 0.291 | 0.070 | 0.236 |
| 24 | 7 | 146510232 | 11972997 | 6574 | TCTGATAATTMCTCTTGCAAT | 0.428 | 0.426 | 0.231 | 0.182 | 0.128 |
| 24 | 7 | 146516306 | 1404709 | 6575 | AAAAAGACTAYGTAAAAAATA | 0.577 | 0.589 | 0.233 | 0.200 | 0.116 |
| 24 | 7 | 146524144 | 700304 | 6576 | CCTTGTTGTCYACCGTTTGCT | 0.015 | 0.014 | 0.386 | 0.303 | 0.142 |
| 24 | 7 | 146533742 | 6963863 | 6577 | ATAAGCCTCCRTTGAAAAGAT | 0.190 | 0.183 | 0.252 | 0.296 | 0.179 |
| 24 | 7 | 146542337 | 4726857 | 6578 | CTGGTACTAAWCTACACAATG | 0.950 | 0.951 | 0.273 | 0.245 | 0.196 |
| 24 | 7 | 146555102 | 10251377 | 6579 | AATTGGTGACRTTAAAGTTAA | 0.030 | 0.029 | 0.320 | 0.265 | 0.185 |
| 24 | 7 | 146562196 | 11773683 | 6580 | ACAGAGGCAAMGGTGACTGAG | 0.676 | 0.678 | 0.344 | 0.249 | 0.258 |
| 24 | 7 | 146567619 | 17170498 | 6581 | TGCTTTCCTTYACTGGCACTT | 0.178 | 0.188 | 0.210 | 0.267 | 0.247 |
| 24 | 7 | 146581895 | 826641 | 6582 | GCTTATTTCAWCCATGATGGA | 0.304 | 0.298 | 0.402 | 0.242 | 0.185 |
| 24 | 7 | 146593236 | 7779225 | 6583 | AAAATGAAGARCTATCACTTA | 0.568 | 0.566 | 0.218 | 0.196 | 0.113 |
| 24 | 7 | 146600422 | 16883479 | 6584 | TGGCTTGATGKGGTCAATTCC | 0.589 | 0.551 | 0.181 | 0.297 | 0.235 |
| 24 | 7 | 146610304 | 4726870 | 6585 | AAGAAAATGGYCCTTTGTCTA | 0.172 | 0.163 | 0.343 | 0.165 | 0.532 |
| 24 | 7 | 146620791 | 2286127 | 6586 | TGTGAGTGCCRATTTATCTCA | 0.056 | 0.057 | 0.296 | 0.136 | 0.423 |
| 24 | 7 | 146627388 | 2141388 | 6587 | AACTTACTTCYCCATCAGTTG | 0.785 | 0.789 | 0.144 | 0.282 | 0.618 |
| 24 | 7 | 146633513 | 1919190 | 6588 | ATTTCAGTTCMCATTCAAAGT | 0.455 | 0.430 | 0.128 | 0.585 | 0.541 |
| 24 | 7 | 146640013 | 6970064 | 6589 | ATCAATATTTRCCCAGTGCAA | 0.035 | 0.035 | 0.406 | 0.555 | 0.578 |
| 24 | 7 | 146646656 | 10282028 | 6590 | CAGGTGCCAAYGTAAACCTAG | 0.089 | 0.083 | 0.756 | 0.767 | 0.925 |
| 24 | 7 | 146655654 | 2888493 | 6591 | GACTCTTCACRGATTTCTGGA | 0.991 | 0.923 | 0.789 | 0.847 | 0.833 |
| 24 | 7 | 146663529 | 1024528 | 6592 | TATGTAAATCYGTTATAATAC | 1.685 | 1.671 | 1.148 | 0.725 | 0.723 |
| 24 | 7 | 146672375 | 12703926 | 6593 | CAATATAAGCRTGACTTAATT | 0.449 | 0.470 | 1.287 | 1.149 | 0.779 |
| 24 | 7 | 146678432 | 4118165 | 6594 | ATGTAATAAGSCAGTAATTGA | 0.826 | 0.832 | 1.013 | 1.163 | 0.838 |
| 24 | 7 | 146684627 | 3915304 | 6595 | CATTATATTTSAATTATTTGG | 0.378 | 0.368 | 0.997 | 1.191 | 0.722 |
| 24 | 7 | 146690849 | 6464821 | 6596 | GGTGTTTCGTRTTCTACTGGT | 0.403 | 0.417 | 0.882 | 1.049 | 0.744 |
| 24 | 7 | 146697376 | 2074712 | 6597 | CTTTTAGATCRTGACAGACCT | 1.701 | 1.626 | 0.664 | 0.664 | 0.925 |
| 24 | 7 | 146711455 | 2189998 | 6598 | TCTTCCACCARTGAAAAGTCA | 0.200 | 0.193 | 0.723 | 0.614 | 1.080 |
| 24 | 7 | 146719444 | 2158640 | 6599 | CCACTATTACYGATGTGAAAA | 0.270 | 0.270 | 0.694 | 0.549 | 1.385 |
| 24 | 7 | 146733806 | 7794693 | 6600 | CTAAAATAAAMTGTCCCAATA | 0.510 | 0.496 | 0.263 | 0.699 | 1.063 |
| 24 | 7 | 146741320 | 7807559 | 6601 | TATTTATTGASCACTAGAGTA | 0.394 | 0.395 | 0.301 | 0.749 | 1.098 |
| 24 | 7 | 146748760 | 988039 | 6602 | ATAAATATCTRACAACAGGTT | 0.287 | 0.292 | 0.546 | 0.874 | 1.208 |
| 24 | 7 | 146751457 | 727716 | 6603 | ATAAGTAAGTRAAATAAACGT | 0.000 | 0.000 | 0.585 | 0.971 | 1.277 |
| 24 | 7 | 146773166 | 17170626 | 6604 | AGGTTCTTAGWGATGCTTCTA | 0.903 | 0.919 | 1.572 | 1.161 | 1.238 |
| 24 | 7 | 146788719 | 10244236 | 6605 | GTATACCAAARGAAGCTTAAT | 0.568 | 0.573 | 1.488 | 1.339 | 0.954 |
| 24 | 7 | 146799052 | 17327617 | 6606 | ATCTCTGGCTRACAGGAAGAG | 2.136 | 2.122 | 1.432 | 1.469 | 0.959 |
| 24 | 7 | 146806955 | 17170638 | 6607 | AGCCATGTTCYTAATACCCAA | 0.305 | 0.311 | 1.410 | 1.384 | 1.055 |
| 24 | 7 | 146815406 | 12672959 | 6608 | CACTAAATATYAGCCATGGAC | 0.525 | 0.534 | 1.573 | 1.352 | 0.882 |
| 24 | 7 | 146819430 | 851735 | 6609 | TCATGCTCTASTCTAAGCTTC | 1.194 | 1.265 | 0.663 | 1.071 | 0.772 |
| 24 | 7 | 146837806 | 17170645 | 6610 | CACTTGAGGCRCATAGCAAGC | 0.532 | 0.518 | 0.800 | 1.041 | 0.811 |
| 24 | 7 | 146844658 | 700317 | 6611 | TGTCCAACTARTTACTAAATT | 0.235 | 0.246 | 0.569 | 0.359 | 0.994 |
| 24 | 7 | 146852926 | 4295581 | 6612 | GGAAGCACAGWTGTATTAGCC | 0.623 | 0.643 | 0.257 | 0.289 | 0.944 |
| 24 | 7 | 146866361 | 4308646 | 6613 | GTCTATTCTGMAGCCTGTGAA | 0.023 | 0.023 | 0.103 | 0.251 | 0.843 |
| 24 | 7 | 146880864 | 17417231 | 6614 | TTTACCTTTAYGTGCTTCCTT | 0.474 | 0.456 | 0.060 | 0.253 | 0.521 |
| 24 | 7 | 146888752 | 10952713 | 6615 | CCCTGCAGCCSTAAGTACCAC | 0.010 | 0.010 | 0.030 | 0.306 | 0.617 |
| 24 | 7 | 146899558 | 17823834 | 6616 | ATCATATCTCYGCTTGGTGCT | 0.017 | 0.017 | 0.347 | 0.266 | 0.721 |
| 24 | 7 | 146926019 | 851697 | 6617 | AATCTATCAGYTCACTTTTCA | 0.398 | 0.396 | 0.454 | 0.329 | 0.514 |
| 24 | 7 | 146935988 | 17226520 | 6618 | TTACTTCTTAKAAACGTGTAT | 1.218 | 1.204 | 0.502 | 0.530 | 0.676 |
| 24 | 7 | 146953728 | 17824995 | 6619 | TTCTAGAGACRTTGAATCCAG | 0.739 | 0.746 | 0.856 | 0.677 | 0.638 |
| 24 | 7 | 146963031 | 17825363 | 6620 | AAAATTGGATYGAAGTCCAGA | 0.097 | 0.101 | 1.017 | 0.820 | 0.890 |
| 24 | 7 | 146971654 | 17170691 | 6621 | ATGTCTTAATYTTTCTCCTCT | 0.914 | 0.828 | 0.887 | 1.244 | 1.079 |
| 24 | 7 | 146975755 | 851705 | 6622 | TTCAAATTCARTTAGGCCTTC | 0.694 | 0.710 | 0.762 | 1.152 | 1.008 |
| 24 | 7 | 146987578 | 10488352 | 6623 | CTGGTTGAAASCTAGACATTT | 0.901 | 0.905 | 1.383 | 1.313 | 1.138 |
| 24 | 7 | 146996863 | 6979528 | 6624 | GCAAACCACTKCAAGGAAGCT | 0.503 | 0.466 | 0.870 | 1.509 | 1.209 |
| 24 | 7 | 147007736 | 10085572 | 6625 | AAATATGAACWTTCAATAAAG | 1.097 | 1.125 | 1.448 | 1.411 | 1.595 |
| 24 | 7 | 147017267 | 2538991 | 6626 | TGTCAACATTMCCCTTGCAGA | 0.065 | 0.069 | 1.360 | 1.212 | 1.305 |
| 24 | 7 | 147023830 | 10488350 | 6627 | GAGACACTGTYAAGTTAAATA | 1.569 | 2.132 | 1.149 | 1.078 | 1.442 |
| 24 | 7 | 147029612 | 17170738 | 6628 | AAGCATTCGCYCTTGTGCTTC | 0.787 | 0.782 | 0.796 | 1.393 | 1.587 |
| 24 | 7 | 147035669 | 2710114 | 6629 | GACTTCTACARTCAATTTGTA | 0.090 | 0.097 | 0.977 | 1.310 | 1.722 |
| 24 | 7 | 147047727 | 17237331 | 6630 | TCTTCAGGGAYGCCTCCTGTA | 0.544 | 0.533 | 0.994 | 1.095 | 1.725 |
| 24 | 7 | 147055375 | 7805223 | 6631 | TCAGCTCATGYCTGAGTCCCT | 0.364 | 0.354 | 0.612 | 1.321 | 1.691 |
| 24 | 7 | 147062628 | 17170749 | 6632 | TACCTACCGAYTATCAATTAA | 1.685 | 1.713 | 0.947 | 1.063 | 1.611 |
| 24 | 7 | 147069783 | 2710093 | 6633 | ACGTCCTTTGSTCATACAGTT | 0.055 | 0.054 | 0.898 | 1.080 | 1.705 |
| 24 | 7 | 147075195 | 10808047 | 6634 | CGATCAATACRATCTTCAAAC | 0.796 | 0.765 | 1.276 | 1.398 | 1.837 |
| 24 | 7 | 147084835 | 1882687 | 6635 | GATAGCCCTGMAGAATCCTGG | 0.302 | 0.458 | 0.820 | 1.192 | 1.210 |
| 24 | 7 | 147093493 | 2708248 | 6636 | AGGAGTCAGAYGTATATTCTA | 1.022 | 1.044 | 1.289 | 1.726 | 1.262 |
| 24 | 7 | 147100208 | 7805706 | 6637 | ATCAGCTATTSAACTTTTTCT | 0.856 | 0.885 | 0.867 | 0.988 | 1.197 |
| 24 | 7 | 147106888 | 7797584 | 6638 | ATTTGTACTTMTATCTATGAG | 0.596 | 0.856 | 1.481 | 1.113 | 1.331 |
| 24 | 7 | 147113950 | 17170767 | 6639 | AAAATGCTACSTGTAAAATTG | 0.031 | 0.031 | 0.948 | 1.212 | 1.204 |
| 24 | 7 | 147127273 | 6958434 | 6640 | GGAATAGATGYCTAGTCCCAA | 1.445 | 1.543 | 0.596 | 0.985 | 0.778 |
| 24 | 7 | 147133198 | 17170777 | 6641 | ATAGCAGGCTRCAGATACAGA | 0.124 | 0.112 | 0.758 | 0.962 | 0.858 |
| 24 | 7 | 147140340 | 2214681 | 6642 | GAGTGGCACCRAGAGGAATGT | 0.285 | 0.296 | 0.756 | 0.675 | 0.659 |
| 24 | 7 | 147153042 | 17170785 | 6643 | ATAGTAATGGWCAAGCTCCCA | 1.104 | 1.047 | 0.569 | 0.516 | 0.622 |
| 24 | 7 | 147160642 | 993715 | 6644 | ATTAAGATGCMTATGTGGCTA | 0.085 | 0.085 | 0.526 | 0.588 | 0.463 |
| 24 | 7 | 147166630 | 4726916 | 6645 | TCACAATAAAYTCTCATTCCC | 1.023 | 0.996 | 0.503 | 0.218 | 0.616 |
| 24 | 7 | 147172614 | 2527059 | 6646 | GGTGGGACAGYGATCCATGTT | 0.035 | 0.036 | 0.238 | 0.259 | 0.526 |
| 24 | 7 | 147178895 | 2707558 | 6647 | TCCGAGTTCAWATCTAACTTG | 0.234 | 0.240 | 0.214 | 0.267 | 0.549 |
| 24 | 7 | 147186296 | 17170811 | 6648 | ACTGATTAGARTGTCAGAATA | 0.326 | 0.324 | 0.048 | 0.398 | 0.327 |
| 24 | 7 | 147192464 | 10485845 | 6649 | TTGATAACCAYTGTATTTCTG | 0.031 | 0.029 | 0.113 | 0.464 | 0.377 |
| 24 | 7 | 147197109 | 10485844 | 6650 | ACTTGAATTCRCAAAATACTC | 0.330 | 0.332 | 0.547 | 0.277 | 0.527 |
| 24 | 7 | 147208831 | 4726920 | 6651 | GAGAATTTCCRTACTACCTCC | 0.391 | 0.377 | 0.572 | 0.318 | 0.418 |
| 24 | 7 | 147218722 | 10485843 | 6652 | CACTCCGTAGRTGACCATAGA | 1.608 | 1.518 | 0.621 | 0.372 | 0.495 |
| 24 | 7 | 147230644 | 17170828 | 6653 | CTCTTTAAGCRTGTCAACTAG | 0.361 | 0.352 | 0.592 | 0.573 | 0.491 |
| 24 | 7 | 147237402 | 11974602 | 6654 | CTGCTCAATAMATGTAGAAGG | 0.159 | 0.163 | 0.638 | 0.694 | 0.490 |
| 24 | 7 | 147243946 | 6962625 | 6655 | ACTCTGCTAARTGTATTTCAT | 0.255 | 0.253 | 0.430 | 0.754 | 0.515 |
| 24 | 7 | 147254702 | 17170844 | 6656 | CTGGATAAGARTAATTGAAAG | 0.000 | 0.000 | 0.497 | 0.959 | 0.567 |
| 24 | 7 | 147259047 | 10228263 | 6657 | ATGGCAAAGARATATTGTTCA | 1.126 | 1.082 | 0.673 | 0.487 | 0.579 |
| 24 | 7 | 147271533 | 1557964 | 6658 | TCACTAAATCRTAACAAAAGT | 0.502 | 0.505 | 1.029 | 0.484 | 0.570 |
| 24 | 7 | 147284643 | 17170855 | 6659 | GCAGAAACAGWAATTTCAGAA | 0.501 | 0.533 | 0.803 | 0.614 | 0.590 |
| 24 | 7 | 147295938 | — | 6660 | TTTTGCTGCCKCTTCATGTCG | 0.965 | 0.946 | 0.447 | 0.556 | 0.345 |
| 24 | 7 | 147302259 | 2972108 | 6661 | GTCAGACATCRGTGCAAAGAA | 0.020 | 0.021 | 0.453 | 0.489 | 0.333 |
| 24 | 7 | 147309435 | 12534650 | 6662 | ACACGCCAAAWTATTAACTGT | 0.351 | 0.344 | 0.285 | 0.289 | 0.454 |
| 24 | 7 | 147316802 | 10260743 | 6663 | TGGCCAAGTGYGTGCCCTTTT | 0.561 | 0.541 | 0.094 | 0.297 | 1.036 |
| 24 | 7 | 147322254 | 2906313 | 6664 | CTACGTAAAAYGAGCTTCTGA | 0.134 | 0.123 | 0.194 | 0.239 | 0.965 |
| 24 | 7 | 147323182 | 13230925 | 6665 | GAGGAGATAGYTAAGATGACT | 0.255 | 0.265 | 0.256 | 0.173 | 0.770 |
| 24 | 7 | 147348080 | 4345484 | 6666 | GTTCTGGCTTYGGGATGGAGT | 0.439 | 0.426 | 0.177 | 0.842 | 0.647 |
| 24 | 7 | 147356671 | 2373346 | 6667 | TAGTGTTTACRTCATGGTGTC | 0.520 | 0.533 | 0.385 | 0.837 | 0.563 |
| 24 | 7 | 147363885 | 1529154 | 6668 | GCTGTCAGCCRATATGGGTCA | 0.266 | 0.282 | 1.223 | 0.830 | 0.391 |
| 24 | 7 | 147374780 | 4726933 | 6669 | ATCGTTTCTGRAGCCTGATTT | 0.687 | 0.665 | 1.183 | 0.789 | 0.533 |
| 24 | 7 | 147380226 | 6945371 | 6670 | ATATTCTGCGYGCTGCTCTTC | 2.201 | 2.132 | 1.250 | 0.795 | 0.481 |
| 24 | 7 | 147388963 | 4590355 | 6671 | CATCAACGGCWCTTACCGCAC | 0.314 | 0.328 | 1.080 | 0.707 | 0.365 |
| 24 | 7 | 147399321 | 17170932 | 6672 | CATTTATTATYAGCAGGCAAC | 0.000 | 0.000 | 0.939 | 0.736 | 1.916 |
| 24 | 7 | 147411605 | 17170936 | 6673 | AATGCACAAARCAGCTCACAT | 0.061 | 0.062 | 0.100 | 0.661 | 2.239 |
| 24 | 7 | 147414418 | 7809486 | 6674 | GTCTTGGCTTRACTAGGACAT | 0.245 | 0.239 | 0.186 | 0.479 | 2.560 |
| 24 | 7 | 147425911 | 7779924 | 6675 | TTCAGAGGCCRTACTGACTTC | 0.256 | 0.256 | 0.113 | 1.651 | 2.537 |
| 24 | 7 | 147437045 | 4726947 | 6676 | TTTAGATGTASATTAAGACCC | 0.639 | 0.649 | 0.110 | 2.252 | 2.672 |
| 24 | 7 | 147464095 | — | 6677 | AGAAGTCCCCRGTGGCTCAGG | 0.116 | 0.115 | 2.735 | 2.362 | 2.946 |
| 24 | 7 | 147473270 | 10281587 | 6678 | CTATCTCTTAYATTGCCCAGT | 0.058 | 0.060 | 3.029 | 2.300 | 1.865 |
| 24 | 7 | 147478836 | 11767934 | 6679 | TTGTCAGATGSTCTTCACACA | 4.904 | 4.364 | 3.526 | 2.788 | 1.909 |
| 24 | 7 | 147485200 | 2037869 | 6680 | TGAGGTCATGYGTAGTAAACA | 1.174 | 1.116 | 3.177 | 2.800 | 1.537 |
| 24 | 7 | 147491445 | — | 6681 | TTTTAGAAGTRTGCTCATTTA | 0.542 | 0.499 | 3.124 | 2.516 | 1.863 |
| 24 | 7 | 147502534 | 1637864 | 6682 | ACATGTCACCRAGAGAAAATG | 0.181 | 0.179 | 1.065 | 2.551 | 1.878 |
| 24 | 7 | 147508766 | 1730398 | 6683 | TATGTGACATRGAGCGTCAGT | 1.382 | 1.369 | 0.585 | 2.251 | 1.993 |
| 24 | 7 | 147515023 | — | 6684 | TAACATACTTYACTTGTAATA | 0.000 | 0.000 | 0.500 | 0.656 | 1.749 |
| 24 | 7 | 147524985 | 4726968 | 6685 | TTTACCTAAAYTTGAGGAGAA | 0.123 | 0.116 | 0.447 | 0.308 | 1.775 |
| 24 | 7 | 147530631 | 10242598 | 6686 | TTCCCCTGCCRCGAATTTCTT | 0.376 | 0.366 | 0.173 | 0.284 | 1.773 |
| 24 | 7 | 147541635 | 10215248 | 6687 | TGCTTCAGTAYATAATCTAAA | 0.033 | 0.033 | 0.091 | 0.276 | 0.468 |
| 24 | 7 | 147555290 | 2530310 | 6688 | AGTTTATTTTYCAGATAAAAT | 0.713 | 0.645 | 0.135 | 0.105 | 0.248 |
| 24 | 7 | 147567741 | — | 6689 | CCAAGGTTTAYGTAAAATAAG | 0.076 | 0.075 | 0.066 | 0.086 | 0.305 |
| 24 | 7 | 147573332 | 2530304 | 6690 | TCTCTAAAAGMAATATAAAAT | 0.000 | 0.000 | 0.263 | 0.184 | 0.298 |
| 24 | 7 | 147581090 | 7780899 | 6691 | GTGCTGCCACYGTCCTTTTGG | 0.125 | 0.128 | 0.120 | 0.139 | 0.442 |
| 24 | 7 | 147594288 | 17616888 | 6692 | TTGACTGAAGRTAGGTTTCAT | 0.617 | 0.580 | 0.340 | 0.328 | 1.033 |
| 24 | 7 | 147600941 | 17171023 | 6693 | TCTGAACCCCRATTCCATGGT | 0.215 | 0.224 | 0.259 | 0.188 | 1.752 |
| 24 | 7 | 147621152 | 6464905 | 6694 | CTTCAATAAAYGGGCAGGTAA | 0.676 | 0.688 | 0.500 | 0.761 | 2.238 |
| 24 | 7 | 147626181 | 6464907 | 6695 | AAGGACTGCTYGACTATTTTA | 0.206 | 0.207 | 0.305 | 1.533 | 2.855 |
| 24 | 7 | 147635310 | 17617268 | 6696 | TGCTGAAAAAKATTCCTTATA | 0.787 | 0.787 | 1.024 | 2.399 | 2.784 |
| 24 | 7 | 147649806 | 2538476 | 6697 | ATTGAAGGCCYCTGGAAAGAG | 0.141 | 0.137 | 1.913 | 2.584 | 3.026 |
| 24 | 7 | 147659874 | 2717817 | 6698 | TTCCATGAACYCTGCTCCAGT | 2.018 | 2.013 | 2.703 | 3.079 | 3.085 |
| 24 | 7 | 147665514 | 11766141 | 6699 | GAAGAACTTTSCAGAAGTAGT | 2.854 | 3.488 | 3.021 | 3.159 | 3.139 |
| 24 | 7 | 147665583 | 11769697 | 6700 | AGTAGAACATRAGGTCATAAT | 2.185 | 2.172 | 3.604 | 3.322 | 2.901 |
| 24 | 7 | 147676361 | 7790220 | 6701 | AAACTTATTTYAGTTATTGAT | 2.142 | 2.515 | 3.604 | 3.474 | 2.816 |
| 24 | 7 | 147695809 | 12704036 | 6702 | TTGATACAGAYTAGAGCCACA | 2.096 | 2.044 | 3.364 | 3.623 | 2.835 |
| 24 | 7 | 147709488 | 11767479 | 6703 | TTTTAAATCTKGCTATTTTAT | 0.479 | 0.453 | 2.979 | 3.386 | 2.872 |
| 24 | 7 | 147725167 | 6960877 | 6704 | ATTGTAGAGGYTTTAGATCAT | 1.368 | 1.857 | 1.426 | 2.185 | 2.652 |
| 24 | 7 | 147747106 | 6959357 | 6705 | TCAAAATGTTRGAACTATACA | 0.491 | 0.484 | 0.574 | 1.606 | 2.778 |
| 24 | 7 | 147758038 | 1015665 | 6706 | GGTTTCAGGAMAATATCAGAA | 0.133 | 0.130 | 0.523 | 1.025 | 2.143 |
| 24 | 7 | 147764071 | 12539045 | 6707 | ACAGGTGCACRGCCCTGAGTC | 0.252 | 0.247 | 0.190 | 0.433 | 1.439 |
| 24 | 7 | 147770295 | 10952770 | 6708 | AAATGAGAATYGCATAGTGCT | 0.274 | 0.272 | 0.253 | 0.339 | 1.115 |
| 24 | 7 | 147777033 | 17171069 | 6709 | TTTATTCTCCYAATATACTTA | 0.000 | 0.000 | 0.193 | 0.109 | 1.088 |
| 24 | 7 | 147790681 | 6963551 | 6710 | GGATAGCCACRGTCTAAGCAT | 0.657 | 0.652 | 0.163 | 0.113 | 0.777 |
| 24 | 7 | 147803868 | 10278049 | 6711 | GAAACAAGTGRGAGTCAGAGT | 0.052 | 0.052 | 0.135 | 0.209 | 0.807 |
| 25 | 7 | 149454345 | 11983355 | 6712 | GGTGCCTTTGMCTTTGGGAAG | 0.649 | 0.622 | 0.167 | 0.479 | 1.283 |
| 25 | 7 | 149463563 | 11764936 | 6713 | AAAATTTAGCKTTCCCAAGTC | 0.488 | 0.476 | 0.452 | 0.353 | 1.268 |
| 25 | 7 | 149471530 | 3735171 | 6714 | GGCGAGCACCRGCTGACCCCC | 0.187 | 0.197 | 0.541 | 0.390 | 0.955 |
| 25 | 7 | 149477203 | 3735167 | 6715 | GTTCCCACACYGCAGGGATCA | 0.949 | 0.925 | 0.552 | 1.199 | 1.331 |
| 25 | 7 | 149483883 | 28432021 | 6716 | GCTGTATTTTSAGAAGTACAA | 0.315 | 0.309 | 0.568 | 1.387 | 1.191 |
| 25 | 7 | 149497250 | 17173682 | 6717 | TGAATAATTTYCACATGACTT | 0.604 | 0.604 | 2.173 | 1.247 | 1.272 |
| 25 | 7 | 149518638 | 2272187 | 6718 | GGGGAGGACCYTGCCTCTTTC | 0.532 | 0.536 | 1.993 | 1.817 | 1.237 |
| 25 | 7 | 149530332 | 4725889 | 6719 | ACTTCTAACTYGTGGGTTCAT | 3.092 | 2.972 | 2.101 | 2.510 | 2.111 |
| 25 | 7 | 149545607 | 1048424 | 6720 | ACATTGAGCCYCGAGTCAAGG | 0.118 | 0.084 | 2.397 | 2.212 | 2.173 |
| 25 | 7 | 149556724 | 2888635 | 6721 | TGCATCGATCKGGTGAGACAC | 0.000 | 0.000 | 2.430 | 1.920 | 4.312 |
| 25 | 7 | 149571403 | 1524335 | 6722 | TGATATACCAYGCAACATTAT | 1.392 | 1.317 | 0.750 | 2.335 | 4.364 |
| 25 | 7 | 149591461 | 7810209 | 6723 | TTATAGTGGGYGATGTGTAAA | 0.600 | 0.592 | 0.638 | 2.414 | 4.312 |
| 25 | 7 | 149602928 | 2373748 | 6724 | TTCTTGCCTAWCCTATTTTAA | 0.776 | 0.793 | 1.585 | 3.503 | 4.187 |
| 25 | 7 | 149624893 | 2222524 | 6725 | ACAATGGGTGRCTTTATTTAT | 0.018 | 0.018 | 1.338 | 3.422 | 4.120 |
| 25 | 7 | 149649666 | 7810752 | 6726 | TGAAAATTCARCTTGTTCAAG | 2.307 | 2.313 | 4.665 | 3.585 | 4.153 |
| 25 | 7 | 149660076 | 7803234 | 6727 | TTTTGTTTTTRTTATTTGTTA | 0.689 | 0.681 | 4.488 | 3.447 | 3.623 |
| 25 | 7 | 149706735 | 13242186 | 6728 | ATCTCTGAGTSCAGCAGTAAC | 4.565 | 4.789 | 4.665 | 4.966 | 3.133 |
| 25 | 7 | 149727819 | 4358715 | 6729 | TCTGCCTCGTWTGGTTTCTGC | 0.101 | 0.100 | 3.206 | 3.748 | 2.656 |
| 25 | 7 | 149738096 | 4367453 | 6730 | TTAAAAAGGTYATTCCTACCC | 0.000 | 0.000 | 4.036 | 5.267 | 2.852 |
| 25 | 7 | 149749542 | — | 6731 | TCTGTTTTTCYCCAGCAGAAG | 0.252 | 0.261 | 0.254 | 2.218 | 3.143 |
| 25 | 7 | 149756449 | 10253121 | 6732 | ATGTGACAGTYTCTAGTTCCC | 0.987 | 1.030 | 0.559 | 1.721 | 2.630 |
| 25 | 7 | 149763839 | 10272462 | 6733 | AGGAATTTGARTGAATGGGCA | 0.107 | 0.102 | 0.450 | 0.601 | 2.812 |
| 25 | 7 | 149768593 | 6946579 | 6734 | TTGCCTAAGTRTTTAGAAAGT | 0.817 | 0.783 | 0.387 | 0.984 | 2.135 |
| 25 | 7 | 149784648 | 10227548 | 6735 | AAGAATCAAASCCCAGTGAAT | 0.261 | 0.250 | 0.663 | 0.998 | 2.209 |
| 25 | 7 | 149795507 | 2373845 | 6736 | TATAGGAAATRTTGGCAATTT | 0.089 | 0.089 | 1.183 | 0.903 | 0.894 |
| 25 | 7 | 149801383 | 16884586 | 6737 | CTCAGCAACAYGGGAAGACAT | 1.708 | 1.646 | 1.097 | 0.774 | 0.955 |
| 25 | 7 | 149825654 | 17173566 | 6738 | TTGTACAGTCYAAGTTCCTTC | 1.215 | 1.154 | 1.025 | 1.114 | 0.957 |
| 25 | 7 | 149852220 | 6464094 | 6739 | GTTAGGAATAYAGGATTCTAT | 0.690 | 0.657 | 1.476 | 1.098 | 1.065 |
| 25 | 7 | 149868951 | 17173583 | 6740 | TTTACCTGCTYTTTGATTTTC | 0.048 | 0.040 | 0.941 | 1.147 | 0.765 |
| 25 | 7 | 149878297 | 6598 | 6741 | TTAGCCAATARATTTCCTACT | 0.686 | 0.671 | 0.690 | 1.386 | 0.771 |
| 25 | 7 | 149894246 | 4725360 | 6742 | AAGAAAAATTRAAAGAACCAA | 0.911 | 0.840 | 0.533 | 0.855 | 0.624 |
| 25 | 7 | 149905239 | 17255187 | 6743 | ACATAGTACAKAATGAATCCT | 0.741 | 0.713 | 0.747 | 0.453 | 0.618 |
| 25 | 7 | 149914536 | 7806458 | 6744 | GTATTAAGACRTAAATGGGAT | 0.299 | 0.303 | 0.673 | 0.285 | 0.681 |
| 25 | 7 | 149921160 | 12669824 | 6745 | AGGAATTAATRCTATCTTCAG | 0.500 | 0.461 | 0.312 | 0.351 | 0.406 |
| 25 | 7 | 149928024 | 2072443 | 6746 | GCAAAGCCTGYCAGGGTGAGC | 0.000 | 0.000 | 0.077 | 0.241 | 0.346 |
| 25 | 7 | 149940256 | 7785283 | 6747 | TCCTAAAACTSCTTGATAAAA | 0.081 | 0.083 | 0.075 | 0.112 | 0.234 |
| 25 | 7 | 149948563 | 2888674 | 6748 | TATGGCCCCARTTGGCAAAGC | 0.050 | 0.048 | 0.034 | 0.101 | 0.228 |
| 25 | 7 | 149955253 | 9640170 | 6749 | ATTTATCTGASTGAAGAATGG | 0.240 | 0.237 | 0.033 | 0.220 | 0.213 |
| 26 | 7 | 150075668 | 2968855 | 6750 | TCAGAGGCATYCCAGAGCATG | 0.183 | 0.179 | 0.023 | 0.049 | 0.224 |
| 26 | 7 | 150104858 | 3807375 | 6751 | AAAAGGAGCAYTCTAAACGCA | 0.155 | 0.158 | 0.035 | 0.004 | 0.303 |
| 26 | 7 | 150107515 | 3778873 | 6752 | ACCTGCAAGTSGAATGGGTTG | 0.105 | 0.111 | 0.051 | 0.121 | 0.238 |
| 26 | 7 | 150118625 | 2373962 | 6753 | AATGCCCTGASAGTTTGCAGA | 0.389 | 0.384 | 0.042 | 0.250 | 0.165 |
| 26 | 7 | 150140429 | 3918188 | 6754 | CGTACAAGGGMGTTTGAGAGA | 0.184 | 0.180 | 0.357 | 0.355 | 0.118 |
| 26 | 7 | 150150382 | 3918216 | 6755 | TATGGGGCCTSTAACAATGAC | 0.123 | 0.122 | 0.663 | 0.348 | 0.601 |
| 26 | 7 | 150166860 | 6947821 | 6756 | TGGAGATACCYGCACTTGCTT | 1.320 | 1.304 | 0.696 | 0.336 | 0.598 |
| 26 | 7 | 150181468 | 4148854 | 6757 | GGGGTGTGATRCAGTGCATTG | 0.902 | 0.891 | 0.682 | 0.463 | 0.813 |
| 26 | 7 | 150195045 | 6464120 | 6758 | TCCAGGCGGGRAGTTCTGTGC | 0.472 | 0.449 | 0.672 | 1.112 | 1.020 |
| 26 | 7 | 150206434 | 2303937 | 6759 | GAGAGAAGACRCAGGACCTGA | 0.134 | 0.134 | 0.383 | 1.147 | 1.172 |
| 26 | 7 | 150212992 | 2303943 | 6760 | ACGAGCTGCAMGTGACTAATC | 0.086 | 0.085 | 1.141 | 1.430 | 2.368 |
| 26 | 7 | 150234900 | 4236428 | 6761 | TGGGGTGTCCYGAGCCCAACT | 0.628 | 0.631 | 1.041 | 1.474 | 2.778 |
| 26 | 7 | 150246812 | 6951528 | 6762 | CCTGGGCTGAMCCAATGAGCA | 2.586 | 2.450 | 1.491 | 1.460 | 3.026 |
| 26 | 7 | 150260307 | 6464131 | 6763 | GGTGCCCGCTSCTTCCCGTTC | 0.301 | 0.294 | 1.945 | 3.317 | 3.434 |
| 26 | 7 | 150269456 | 6979622 | 6764 | TGTGCCCTAAYGCAGCCCTCG | 1.000 | 0.949 | 2.042 | 3.526 | 3.250 |
| 26 | 7 | 150286812 | 6464132 | 6765 | GCTTCGTGAARGTAGGTATTC | 0.982 | 0.926 | 2.721 | 3.467 | 3.031 |
| 26 | 7 | 150295919 | 4725395 | 6766 | CTTCTTTCCARTTAAAATATT | 0.752 | 0.759 | 2.681 | 4.036 | 2.701 |
| 26 | 7 | 150312660 | 4726005 | 6767 | ACACAGAAGTYATCACTCACA | 4.390 | 3.835 | 2.450 | 2.486 | 3.217 |
| 26 | 7 | 150320869 | 2257069 | 6768 | GCAATTTCCCRATGCGGGCCA | 1.294 | 1.226 | 3.665 | 2.584 | 3.364 |
| 26 | 7 | 150348102 | 2608288 | 6769 | AGTAAAGGGASAAAGTTCTTC | 0.174 | 0.160 | 3.375 | 2.221 | 4.225 |
| 26 | 7 | 150355235 | 7784344 | 6770 | GGCTATATTAWAAACTAATAC | 0.953 | 0.947 | 0.584 | 3.153 | 2.533 |
| 26 | 7 | 150362605 | 3807386 | 6771 | CCCGCTGCCGMATTGTTAGGT | 0.097 | 0.087 | 0.154 | 3.375 | 3.245 |
| 26 | 7 | 150373700 | 1870238 | 6772 | AATGACCACASGGCTGCTGTC | 0.308 | 0.306 | 0.480 | 0.711 | 2.743 |
| 26 | 7 | 150402025 | 7799570 | 6773 | GACTATTACTRTATCCATTTT | 0.030 | 0.030 | 0.366 | 0.360 | 2.838 |
| 26 | 7 | 150429874 | 219254 | 6774 | TAAATCTGTGRCTGAAATCTT | 1.154 | 1.116 | 0.535 | 1.405 | 2.641 |
| 26 | 7 | 150432996 | 219271 | 6775 | CCATCATGGGYGTGGCCACAG | 0.643 | 0.715 | 0.507 | 0.943 | 0.835 |
| 26 | 7 | 150444177 | 6964641 | 6776 | TGGAAACCTCRAAGCTACATC | 0.436 | 0.421 | 1.958 | 1.009 | 0.510 |
| 26 | 7 | 150454523 | 17705372 | 6777 | TTTCAAATACRTCTCTGACCC | 0.243 | 0.237 | 1.272 | 0.924 | 0.606 |
| 26 | 7 | 150457561 | 760234 | 6778 | CCTCTGCTTTYTAAGGGACAT | 2.577 | 2.567 | 0.988 | 0.985 | 0.356 |
| 26 | 7 | 150478042 | 2063995 | 6779 | GTCCTGATTARCCTAATTCTT | 0.078 | 0.076 | 0.858 | 0.589 | 0.375 |
| 26 | 7 | 150484480 | 414168 | 6780 | GGACTGATCGRTGCCAGCACT | 0.270 | 0.272 | 0.806 | 0.497 | 0.337 |
| 26 | 7 | 150491281 | 11771465 | 6781 | TACTAGAGAAMCTTGTCCCCT | 0.051 | 0.052 | 0.013 | 0.372 | 0.791 |
| 26 | 7 | 150504189 | 13234689 | 6782 | AAAATTGTTTYTTCATGAGGT | 0.170 | 0.163 | 0.044 | 0.335 | 0.579 |
| 26 | 7 | 150532779 | 12703130 | 6783 | TATCTACACARAGCTATGGGT | 0.175 | 0.176 | 0.020 | 0.003 | 0.418 |
| 26 | 7 | 150540515 | 4726024 | 6784 | TTCACAGGGARTCTCCTATGC | 0.354 | 0.425 | 0.025 | 0.132 | 0.428 |
| 26 | 7 | 150561177 | 17712606 | 6785 | ACTGATAACCRGTCATTGCCA | 0.071 | 0.069 | 0.028 | 0.152 | 0.365 |
| 27 | 7 | 156602277 | 6976552 | 6786 | TTAAATTTCTYCGCAGCTTCA | 0.599 | 0.547 | 0.913 | 0.846 | 0.725 |
| 27 | 7 | 156641263 | 4716470 | 6787 | TGTTGCTCACSCTTATCTGGA | 0.452 | 0.452 | 0.548 | 0.582 | 0.565 |
| 27 | 7 | 156664996 | 4716472 | 6788 | TGAGGCTGAGRTGTGTCCCGT | 0.249 | 0.256 | 0.171 | 0.433 | 0.566 |
| 27 | 7 | 156681516 | 10255470 | 6789 | ATCTCTGTTAYTGATTGGCAG | 0.006 | 0.006 | 0.057 | 0.296 | 0.603 |
| 27 | 7 | 156693363 | 10440911 | 6790 | TCTTCAGTCGYGTACATGATT | 0.044 | 0.045 | 0.047 | 0.067 | 0.652 |
| 27 | 7 | 156700081 | 6952964 | 6791 | CCTGGTCGGGYGCCCTGGCAT | 0.106 | 0.109 | 0.045 | 0.033 | 0.514 |
| 27 | 7 | 156705616 | 17837758 | 6792 | TCAACAGCTCKAAGTTTGTCT | 0.490 | 0.451 | 0.041 | 0.051 | 0.363 |
| 27 | 7 | 156709697 | 2072421 | 6793 | TTACGATTACRAAGGATGAAA | 0.358 | 0.360 | 0.068 | 0.196 | 0.197 |
| 27 | 7 | 156728759 | 10242467 | 6794 | ACAATACAGCMTGGAATTTGA | 0.040 | 0.040 | 0.227 | 0.300 | 0.242 |
| 27 | 7 | 156758455 | 10807645 | 6795 | TGTTTTATGCYATATCATTTC | 0.254 | 0.239 | 0.580 | 0.356 | 0.233 |
| 27 | 7 | 156760145 | 2366564 | 6796 | CTAATGAAGTRTGTAAACCTC | 0.749 | 0.724 | 0.647 | 0.473 | 0.865 |
| 27 | 7 | 156779766 | 221216 | 6797 | GATATCACGGYGGATGAGAGT | 1.367 | 1.354 | 0.784 | 0.605 | 1.493 |
| 27 | 7 | 156789143 | 758925 | 6798 | TCCAAATCCTYGGCCCGACAT | 0.000 | 0.000 | 1.017 | 0.662 | 1.553 |
| 27 | 7 | 156795541 | 17667159 | 6799 | GTTCCAGGGAMGTGCTGCTGA | 0.235 | 0.237 | 1.013 | 1.712 | 1.732 |
| 27 | 7 | 156808554 | 1468649 | 6800 | TGGGCGGGTGMAGCAGCCACC | 0.000 | 0.000 | 0.486 | 2.286 | 1.728 |
| 27 | 7 | 156814282 | 10260168 | 6801 | TCCCTGGCCTWTTGTAAATAA | 0.794 | 0.792 | 1.604 | 2.267 | 1.804 |
| 27 | 7 | 156821109 | 221232 | 6802 | CCACACAAGCYGCATGGCCAA | 0.358 | 0.555 | 2.484 | 2.041 | 1.873 |
| 27 | 7 | 156834693 | 221258 | 6803 | GTGCTAATCCYGCAGAGCTGC | 2.581 | 2.463 | 2.243 | 1.884 | 1.899 |
| 27 | 7 | 156838212 | 221271 | 6804 | TTCGCATGGTYCCATTTCCTT | 2.101 | 2.287 | 2.353 | 2.033 | 1.955 |
| 27 | 7 | 156862087 | 221295 | 6805 | AAAGATGTGAMTACCCAGCAG | 0.687 | 0.683 | 2.054 | 1.815 | 1.561 |
| 27 | 7 | 156866999 | 221300 | 6806 | TGTGAAGCTTYCTGAGAATGT | 0.900 | 0.910 | 1.387 | 1.791 | 1.465 |
| 27 | 7 | 156881375 | 2021743 | 6807 | GGTGGAATGAYGGGAGGCTCA | 0.306 | 0.300 | 0.708 | 1.762 | 1.743 |
| 27 | 7 | 156889073 | 17837784 | 6808 | CAAATTTTAAYCCTATGCAAT | 0.877 | 0.850 | 0.566 | 1.009 | 1.775 |
| 27 | 7 | 156895693 | 17837785 | 6809 | GGTCTCTTTCRTAAGAAACAG | 0.287 | 0.291 | 0.441 | 0.417 | 1.741 |
| 27 | 7 | 156912409 | — | 6810 | ATTGGAACCTRCTATACACAT | 0.319 | 0.295 | 0.328 | 0.541 | 1.746 |
| 27 | 7 | 156919171 | 1263563 | 6811 | TAACAGCAGTYGTATAAATTT | 0.638 | 0.636 | 0.097 | 0.415 | 1.086 |
| 27 | 7 | 156927403 | 17837786 | 6812 | GCCTGGTACAMCCTACATGAT | 0.020 | 0.021 | 0.371 | 0.454 | 0.654 |
| 27 | 7 | 156934044 | 17837788 | 6813 | AGCTACCTGAYGTCTGGCTCA | 0.174 | 0.125 | 0.466 | 0.426 | 0.589 |
| 27 | 7 | 156952480 | 1638027 | 6814 | TGTGATTTGCYACCCATGGGA | 1.120 | 1.131 | 0.359 | 0.462 | 0.817 |
| 27 | 7 | 156964490 | 17731604 | 6815 | ACGTTTGGGGMTCACTTAACA | 0.530 | 0.499 | 0.734 | 0.520 | 1.240 |
| 27 | 7 | 156969688 | 17837795 | 6816 | GTACACTGCTYTTGGAAACTG | 0.395 | 0.396 | 0.863 | 0.472 | 1.047 |
| 27 | 7 | 156992288 | 7811860 | 6817 | AAGAAAACCARAAATCTAGTC | 0.783 | 0.784 | 0.535 | 1.204 | 1.116 |
| 27 | 7 | 157005136 | 7802459 | 6818 | TAAACGCCTGYTGTTTGAAGG | 0.388 | 0.374 | 0.501 | 1.802 | 1.095 |
| 27 | 7 | 157013152 | 7792857 | 6819 | TGGGGAGGTARGTGTGGGGGC | 0.000 | 0.000 | 1.144 | 1.365 | 1.156 |
| 27 | 7 | 157043714 | 1796286 | 6820 | TCTCTGTCCTYGGCGTGGGAG | 0.471 | 0.466 | 1.951 | 1.345 | 1.212 |
| 27 | 7 | 157070372 | 6949185 | 6821 | CCAACGCTCCRCCTGCATGGC | 1.724 | 1.703 | 1.666 | 1.300 | 1.155 |
| 27 | 7 | 157082642 | 4716494 | 6822 | GAAGATCAGCRTTCAGACCAG | 1.474 | 1.632 | 1.666 | 1.363 | 0.963 |
| 27 | 7 | 157093892 | 7783909 | 6823 | CAGGAGAGGCRGCTGCTGAAT | 0.210 | 0.207 | 1.568 | 1.270 | 1.781 |
| 27 | 7 | 157104944 | 870020 | 6824 | CCCACCACAGRTGGGTAGCTG | 0.000 | 0.000 | 0.810 | 1.012 | 1.737 |
| 27 | 7 | 157134398 | 7777198 | 6825 | CCAGGCCTCCYCCACACATTC | 0.249 | 0.241 | 0.187 | 1.036 | 1.922 |
| 27 | 7 | 157145415 | 1560962 | 6826 | AGACCCAGAGRCTCACGCTCA | 0.691 | 0.659 | 0.144 | 1.578 | 1.771 |
| 27 | 7 | 157158172 | 7788108 | 6827 | GCTGCCGCGCMACCTCTGACA | 0.211 | 0.207 | 0.144 | 0.818 | 1.621 |
| 27 | 7 | 157182316 | 731305 | 6828 | ATACATTTCTYGGAGTTTGAT | 0.043 | 0.044 | 1.440 | 1.171 | 1.400 |
| 27 | 7 | 157198659 | 4716509 | 6829 | GTGAGGCCCAYGAGGGGCCCC | 0.000 | 0.000 | 1.135 | 1.002 | 1.520 |
| 27 | 7 | 157213555 | 6459817 | 6830 | CTCTGTTTGAYGCTATGATGA | 2.727 | 2.620 | 1.684 | 1.025 | 1.458 |
| 27 | 7 | 157226309 | 1991963 | 6831 | CCATCTTGCCRCTGGTGACGT | 0.175 | 0.179 | 1.693 | 0.864 | 1.627 |
| 27 | 7 | 157237334 | 6977123 | 6832 | CGGAGGCCCCRGTGCTGGGTG | 1.121 | 1.076 | 1.484 | 1.729 | 1.724 |
| 27 | 7 | 157250123 | 6955761 | 6833 | CTAAGAACCARTATCCCTTAT | 0.261 | 0.274 | 0.280 | 2.515 | 1.736 |
| 27 | 7 | 157265078 | 4716857 | 6834 | ACTCCACACCRTGAGAACAGG | 0.089 | 0.091 | 1.099 | 2.515 | 1.725 |
| 27 | 7 | 157277737 | 1036373 | 6835 | GATGAAAATGYAGTTATACCA | 0.257 | 0.258 | 1.330 | 1.434 | 1.944 |
| 27 | 7 | 157280884 | 1347391 | 6836 | AGGTCATGCAYGTCCACACTT | 2.016 | 2.029 | 1.599 | 1.364 | 2.384 |
| 27 | 7 | 157328316 | 6951695 | 6837 | AATTAGGGGCRTAACAATAAC | 1.300 | 1.317 | 1.823 | 1.105 | 2.384 |
| 27 | 7 | 157342999 | 3935208 | 6838 | CTCTTTATGCMAAGAGGCACT | 0.000 | 0.000 | 1.785 | 1.261 | 1.437 |
| 27 | 7 | 157353794 | 7455546 | 6839 | ACGGAGGAAGSCTGTGGCTCC | 0.855 | 0.857 | 0.945 | 1.641 | 1.557 |
| 27 | 7 | 157368860 | 1806465 | 6840 | CCTCTCACCTKCAGCCCCTGA | 0.142 | 0.134 | 0.586 | 1.798 | 1.402 |
| 27 | 7 | 157379611 | 7780849 | 6841 | AAGAGCAGGARGAGTTTCTGA | 0.564 | 0.570 | 0.935 | 1.192 | 1.304 |
| 27 | 7 | 157405658 | 2335844 | 6842 | AGGAGTAGGASAATGGTAACA | 0.636 | 0.616 | 0.797 | 0.839 | 1.436 |
| 27 | 7 | 157418058 | 4909094 | 6843 | GGCTGCACCCYGTCCTGGCAC | 1.172 | 1.253 | 0.936 | 0.889 | 1.738 |
| 27 | 7 | 157434414 | 4909107 | 6844 | ACTCCAGGCCRCTGCCCTAGT | 0.000 | 0.000 | 0.946 | 0.648 | 1.040 |
| 27 | 7 | 157469377 | 2335836 | 6845 | TTGCATTCCCSCTAATTTGAG | 0.355 | 0.373 | 1.004 | 0.819 | 0.694 |
| 27 | 7 | 157493079 | 6963290 | 6846 | AAATTATTTAYGTGATTTCAT | 0.000 | 0.000 | 0.396 | 0.998 | 0.610 |
| 27 | 7 | 157500728 | 6942903 | 6847 | CAACAAAAATRCATTTCAAAT | 0.727 | 0.712 | 0.396 | 0.764 | 0.555 |
| 27 | 7 | 157525134 | 6950346 | 6848 | CCACAGCTAAYACCATAATGA | 0.235 | 0.237 | 0.737 | 0.368 | 0.660 |
| 27 | 7 | 157536873 | 4909053 | 6849 | GTGTGTGTGGKTGTGAGTTGT | 0.000 | 0.000 | 0.485 | 0.301 | 0.555 |
| 27 | 7 | 157548897 | 2001763 | 6850 | TTTGCCCGAGYTTAAAATTAA | 0.992 | 0.969 | 0.281 | 0.397 | 0.518 |
| 27 | 7 | 157564283 | 4909164 | 6851 | GTGAAAATGAYCACAGGGTGC | 0.067 | 0.068 | 0.288 | 0.397 | 0.292 |
| 27 | 7 | 157577455 | 896769 | 6852 | CTGTGCTATGWAGGGACAGGA | 0.266 | 0.273 | 0.393 | 0.250 | 0.508 |
| 27 | 7 | 157590205 | 6950401 | 6853 | TAGTGTTTTCYTACGATTCTT | 0.204 | 0.210 | 0.199 | 0.313 | 0.429 |
| 27 | 7 | 157602000 | 7790889 | 6854 | CCCAAACGTAMGTGCTGGAAT | 0.670 | 0.673 | 0.264 | 0.286 | 0.454 |
| 27 | 7 | 157616338 | 4909064 | 6855 | GTCTCCGCATRGACAAGACGG | 0.000 | 0.000 | 0.296 | 0.388 | 0.374 |
| 27 | 7 | 157619962 | 7782393 | 6856 | TGTAAATCCCRGTGCCCTGGG | 0.244 | 0.238 | 0.359 | 0.398 | 0.600 |
| 27 | 7 | 157654735 | 6459868 | 6857 | TGTCCCCTCTRCAAGGGACAA | 0.000 | 0.000 | 0.686 | 0.481 | 0.518 |
| 27 | 7 | 157667519 | 4909077 | 6858 | AGGCACAATGWGGGCAGCAGA | 0.334 | 0.325 | 0.464 | 0.531 | 0.381 |
| 27 | 7 | 157681505 | 896762 | 6859 | AAGTGAAAGCRCACTTTGACT | 1.344 | 1.323 | 0.645 | 0.690 | 0.829 |
| 27 | 7 | 157693852 | 4909210 | 6860 | TAGTAAAATAYCTTGATGATC | 0.065 | 0.067 | 0.559 | 0.572 | 0.809 |
| 27 | 7 | 157696513 | 4909212 | 6861 | ATGCTGAGGAKACAGAGATTC | 0.599 | 0.580 | 0.969 | 0.628 | 1.044 |
| 27 | 7 | 157716083 | 4909085 | 6862 | TTCTGCTGTGSTCTGTAGTTT | 0.358 | 0.322 | 0.410 | 1.124 | 1.555 |
| 27 | 7 | 157731429 | 1476199 | 6863 | CACCTCCTGCRCATGCCTCAG | 1.173 | 1.143 | 0.530 | 1.064 | 2.450 |
| 27 | 7 | 157784389 | 7788773 | 6864 | GTCCCTGGTCMCGGCTCTGGC | 0.184 | 0.179 | 1.153 | 0.937 | 2.535 |
| 27 | 7 | 157888770 | 1882247 | 6865 | GAGAAGCAACMTGTGATAATC | 0.350 | 0.354 | 1.019 | 1.689 | 2.535 |
| 27 | 7 | 157900851 | 6972776 | 6866 | GGAATCCCCARTGCCAACACA | 1.792 | 1.868 | 0.973 | 2.434 | 2.397 |
| 27 | 7 | 157910068 | 7788516 | 6867 | CTCACTAACTKGAGGAGATGG | 0.220 | 0.222 | 1.752 | 2.471 | 2.178 |
| 27 | 7 | 157927271 | 4909243 | 6868 | AATCAATTTTYTGAGAAAAAC | 1.010 | 1.014 | 2.544 | 2.349 | 2.192 |
| 27 | 7 | 157939751 | 3793177 | 6869 | TTTCCACCTGYAATAGCTGAG | 2.484 | 2.320 | 2.296 | 2.308 | 2.129 |
| 27 | 7 | 157963460 | 2290391 | 6870 | AAATACAATGSAAGGCAGGGG | 3.835 | 3.488 | 2.698 | 2.295 | 2.359 |
| 27 | 7 | 157979187 | 963177 | 6871 | GAAGCCACCCYGGCCAGGCCA | 0.000 | 0.000 | 2.816 | 1.930 | 2.193 |
| 27 | 7 | 157991785 | 1037458 | 6872 | CCAGGCCCACYGTGCTCTCCA | 0.000 | 0.000 | 2.243 | 2.290 | 2.240 |
| 27 | 7 | 158007683 | 7799553 | 6873 | AATTATAGGCYGAGTGAGGTG | 0.009 | 0.009 | 0.377 | 2.713 | 2.177 |
| 27 | 7 | 158031369 | 7800556 | 6874 | TACACGATAARATAATTTCTA | 0.352 | 0.325 | 0.377 | 2.531 | 1.906 |
| 27 | 7 | 158044510 | 1189191 | 6875 | AGCCATGAAGKTGTTGACTCA | 0.922 | 0.910 | 1.325 | 1.205 | 2.096 |
| 27 | 7 | 158055182 | 844521 | 6876 | TTAAAACATAYTTTTGATTAT | 0.000 | 0.000 | 1.710 | 1.098 | 2.061 |
| 27 | 7 | 158071349 | 842444 | 6877 | GATCAAAGGCRTCAAACATAG | 2.545 | 2.455 | 1.588 | 0.947 | 1.762 |
| 27 | 7 | 158084298 | 7780170 | 6878 | CGGCTACTTTMCTGAATTTAT | 0.776 | 0.750 | 1.440 | 1.154 | 1.010 |
| 27 | 7 | 158097267 | 2279881 | 6879 | ACATCAGTGGSGATCTCTGCT | 0.133 | 0.221 | 1.194 | 1.255 | 1.040 |
| 27 | 7 | 158116469 | 1154002 | 6880 | TTCAGGTGCTRAAGAAGCACT | 0.422 | 0.420 | 0.280 | 1.068 | 0.950 |
| 28 | 9 | 26700092 | 10511781 | 6881 | CACCAGTTCARTCACCAATGT | 1.189 | 1.192 | 0.651 | 0.737 | 1.002 |
| 28 | 9 | 26714384 | 7866709 | 6882 | ACCTTCTGTAWGTTCAAGTTC | 1.351 | 1.363 | 0.610 | 0.386 | 1.098 |
| 28 | 9 | 26715878 | 7028218 | 6883 | CAAGTGGGAAYGGCAGAACTG | 0.039 | 0.039 | 0.687 | 0.412 | 0.870 |
| 28 | 9 | 26731583 | 17693460 | 6884 | ACCATGCCCCRGGTCCTGTGC | 0.120 | 0.118 | 0.337 | 0.350 | 0.635 |
| 28 | 9 | 26739795 | 10967513 | 6885 | ATCACTTAATMACATTCATGA | 0.407 | 0.381 | 0.073 | 0.616 | 0.616 |
| 28 | 9 | 26749215 | 7871924 | 6886 | AAATTTGCCTRATCTGGTCTC | 0.054 | 0.053 | 0.056 | 0.310 | 0.567 |
| 28 | 9 | 26757842 | 12350711 | 6887 | CTATTTTGGAYTGCTTTGATG | 0.531 | 0.535 | 0.320 | 0.171 | 0.550 |
| 28 | 9 | 26766151 | 17760488 | 6888 | TTACCTACAAMCTTGAGGAAG | 0.030 | 0.029 | 0.227 | 0.480 | 0.656 |
| 28 | 9 | 26773300 | 17693816 | 6889 | ATGGAAAACAYGGAAAGGAGA | 1.113 | 1.146 | 0.473 | 0.534 | 0.452 |
| 28 | 9 | 26779940 | 1889899 | 6890 | AAGAGTAGTTRGTTGACCAGT | 0.044 | 0.045 | 0.883 | 0.456 | 0.317 |
| 28 | 9 | 26786160 | 10116852 | 6891 | TTGTTTGTTCRTTCATTTTCC | 0.733 | 0.742 | 0.976 | 0.676 | 0.330 |
| 28 | 9 | 26791741 | 17694003 | 6892 | GGGGTTATACKCTCTGCTTAG | 1.369 | 1.374 | 0.526 | 0.557 | 0.393 |
| 28 | 9 | 26798068 | 12000467 | 6893 | TACAATACACSCTGACATTAT | 0.226 | 0.231 | 0.828 | 0.725 | 0.356 |
| 28 | 9 | 26816017 | 10967550 | 6894 | GCTAGTTGTGRTATAGAAAAT | 0.143 | 0.146 | 0.560 | 0.446 | 0.391 |
| 28 | 9 | 26822939 | 17760657 | 6895 | CATGAAAGTCRGTCTCCTCAC | 0.750 | 0.752 | 0.292 | 0.539 | 0.341 |
| 28 | 9 | 26829501 | 17694087 | 6896 | GTGTTCAACARAAAGATGTGT | 0.206 | 0.197 | 0.255 | 0.405 | 0.347 |
| 28 | 9 | 26847244 | 1889901 | 6897 | TGCACACATAYACCTACACAT | 0.643 | 0.640 | 0.375 | 0.143 | 0.269 |
| 28 | 9 | 26856642 | 7036871 | 6898 | CTTTGAAGCTYGGGACCTTAA | 0.115 | 0.116 | 0.197 | 0.146 | 0.399 |
| 28 | 9 | 26868374 | 10511791 | 6899 | TTGGCGCTCASGTTTAGTAGC | 0.410 | 0.416 | 0.163 | 0.149 | 0.293 |
| 28 | 9 | 26876393 | 7857990 | 6900 | GAAGGGAATGYATAAACTTGT | 0.172 | 0.171 | 0.083 | 0.131 | 0.118 |
| 28 | 9 | 26883662 | 7038314 | 6901 | CAGGAGTTCCRTTAAGTATCA | 0.221 | 0.222 | 0.066 | 0.230 | 0.175 |
| 28 | 9 | 26889130 | 7860542 | 6902 | CTCAACACCTRATACATTACC | 0.264 | 0.262 | 0.089 | 0.118 | 0.267 |
| 28 | 9 | 26894699 | 16910873 | 6903 | TACAGTTTGAKGTACTCTTGC | 0.020 | 0.020 | 0.278 | 0.097 | 0.193 |
| 28 | 9 | 26900951 | 10967594 | 6904 | TTATTCCAAAKGATGTAGTAG | 0.571 | 0.562 | 0.232 | 0.142 | 0.176 |
| 28 | 9 | 26909666 | 16910888 | 6905 | TAAAATCTTCSTTTTTGGCAG | 0.852 | 0.826 | 0.182 | 0.265 | 0.395 |
| 28 | 9 | 26914621 | 10511793 | 6906 | CTTTCCATTCYTTGACTCCTC | 0.009 | 0.010 | 0.392 | 0.269 | 0.406 |
| 28 | 9 | 26925996 | 7045881 | 6907 | CAACCAGCAGWTTATGATTTA | 0.055 | 0.053 | 0.450 | 0.255 | 0.342 |
| 28 | 9 | 26938134 | 10511794 | 6908 | CCCCTGGTTTSGGAGATCATT | 0.655 | 0.665 | 0.204 | 0.694 | 1.014 |
| 28 | 9 | 26946868 | 7857396 | 6909 | CAGTTAGCAASTTTCCACTTA | 0.717 | 0.775 | 0.251 | 0.600 | 0.952 |
| 28 | 9 | 26952769 | 12352441 | 6910 | GACAGAAATARCAAGTACTGA | 0.094 | 0.090 | 0.922 | 0.437 | 0.973 |
| 28 | 9 | 26980826 | 17694499 | 6911 | TGAGTTAACTWGGGATACTCA | 0.104 | 0.101 | 0.769 | 1.478 | 1.269 |
| 28 | 9 | 26994338 | 10967655 | 6912 | TCAGCTCAAGRTAGATTCTGT | 2.095 | 2.085 | 0.530 | 1.439 | 1.235 |
| 28 | 9 | 26999738 | 17756299 | 6913 | CTGCTTAATAMACAGCTAACC | 0.212 | 0.202 | 2.534 | 1.485 | 1.221 |
| 28 | 9 | 27008020 | 12337896 | 6914 | AATCATTTCARAAAGGTATTA | 0.055 | 0.057 | 2.842 | 1.735 | 1.216 |
| 28 | 9 | 27018947 | 7390085 | 6915 | TGGAAAAGATRTCTAACCTCC | 4.340 | 4.062 | 1.692 | 2.035 | 1.341 |
| 28 | 9 | 27031315 | 7045747 | 6916 | ACATTCCTGTYAGAGCACTTA | 0.052 | 0.052 | 2.756 | 2.499 | 1.324 |
| 28 | 9 | 27037460 | 16910953 | 6917 | GGCTCACTATYGTATCTTCTC | 0.171 | 0.180 | 2.848 | 1.503 | 1.194 |
| 28 | 9 | 27040593 | 1413824 | 6918 | CCTGGAATTAMTACAGTTAGT | 1.388 | 1.387 | 0.358 | 1.719 | 1.288 |
| 28 | 9 | 27057529 | 10511798 | 6919 | AGAAAGAACTRTTATTAAATT | 0.063 | 0.062 | 0.393 | 1.804 | 1.318 |
| 28 | 9 | 27071879 | 16910976 | 6920 | TTATAATGCTMATCCAAGCCA | 0.437 | 0.466 | 0.540 | 0.216 | 1.016 |
| 28 | 9 | 27088940 | 1161603 | 6921 | AAGGTGAATARAAATGTAACT | 0.148 | 0.132 | 0.132 | 0.210 | 0.991 |
| 28 | 9 | 27096614 | 10967715 | 6922 | TTGGGGGCACRTATTTGATTT | 0.549 | 0.538 | 0.191 | 0.196 | 1.103 |
| 28 | 9 | 27104804 | 680739 | 6923 | AACCAGGGCCWTACTTAAGAT | 0.253 | 0.251 | 0.081 | 0.120 | 0.117 |
| 28 | 9 | 27115056 | 12339737 | 6924 | CCCAACCAGCRTGGGCTTGCA | 0.247 | 0.281 | 0.090 | 0.127 | 0.263 |
| 28 | 9 | 27122514 | 7044842 | 6925 | ACAGTGTCTTYCCTGGATATT | 0.005 | 0.006 | 0.203 | 0.110 | 0.278 |
| 28 | 9 | 27129817 | 10967731 | 6926 | TAGTACAGCARGAGTAACAAC | 0.150 | 0.154 | 0.146 | 0.112 | 0.129 |
| 28 | 9 | 27143055 | 7855848 | 6927 | AGGATTGTTAWGCTTCACTTG | 1.000 | 0.989 | 0.155 | 0.204 | 0.234 |
| 28 | 9 | 27153395 | 506685 | 6928 | CTATGAGGGCRTCTAACCTGT | 0.059 | 0.060 | 0.201 | 0.213 | 0.303 |
| 28 | 9 | 27159124 | 3818283 | 6929 | GCGATTATAGRTATCTCCTGG | 0.351 | 0.337 | 0.562 | 0.244 | 0.694 |
| 28 | 9 | 27166229 | 11791294 | 6930 | TATGTATCAGKGCTGGTTTCT | 0.162 | 0.166 | 0.276 | 0.443 | 0.650 |
| 28 | 9 | 27175762 | 17834811 | 6931 | CTTTAAAAAAKTATATGCATG | 1.062 | 1.090 | 0.412 | 0.649 | 0.600 |
| 28 | 9 | 27220196 | 1407300 | 6932 | CTCATAGCTAYTGCCACATGT | 0.273 | 0.268 | 0.580 | 0.993 | 1.129 |
| 28 | 9 | 27229343 | 17695236 | 6933 | CTGTGGAAATRTAACCTTTAA | 0.444 | 0.430 | 0.876 | 1.130 | 1.296 |
| 28 | 9 | 27240868 | 4879313 | 6934 | ACTTGGCTTAKTTAGGGAATA | 0.763 | 0.743 | 1.324 | 0.967 | 1.297 |
| 28 | 9 | 27249071 | 17695332 | 6935 | TGGCTTCCTTRCTTTATCCTG | 0.827 | 0.838 | 1.429 | 1.884 | 1.677 |
| 28 | 9 | 27257825 | 10812556 | 6936 | GAGGGATGAARAAAATCTTAA | 1.883 | 1.978 | 1.186 | 1.635 | 1.793 |
| 28 | 9 | 27264428 | 13288748 | 6937 | CGCTGATTGTRATCCAATGCA | 0.383 | 0.377 | 2.188 | 1.662 | 1.610 |
| 28 | 9 | 27273000 | 10967855 | 6938 | ATTTATCTGAYTTACCTGAAC | 0.056 | 0.057 | 1.895 | 2.387 | 1.795 |
| 28 | 9 | 27283009 | 7873922 | 6939 | TGGGTCCCTCYAGAGGAATGT | 1.909 | 1.914 | 0.740 | 2.210 | 1.843 |
| 28 | 9 | 27289620 | 17778819 | 6940 | ACAAATTAAGYCCAACTTGTG | 0.431 | 0.414 | 1.575 | 1.705 | 1.705 |
| 28 | 9 | 27297163 | 10967874 | 6941 | ACTGGCTGAGYGGAACCTGTC | 0.241 | 0.244 | 1.568 | 0.936 | 1.671 |
| 29 | 9 | 70132969 | 2182739 | 6942 | TTCTTAATTCRTTTTTCTGCT | 0.108 | 0.102 | 0.530 | 0.178 | 0.333 |
| 29 | 9 | 70141432 | 7038061 | 6943 | TAAAATCTCASAATATTAGTC | 0.188 | 0.183 | 0.285 | 0.204 | 0.428 |
| 29 | 9 | 70150928 | 17052748 | 6944 | AAATTATACARTGGACTCCAA | 0.604 | 0.595 | 0.055 | 0.343 | 0.420 |
| 29 | 9 | 70163512 | 7025228 | 6945 | AATAACAACASCATTTCCACA | 0.135 | 0.137 | 0.060 | 0.467 | 0.620 |
| 29 | 9 | 70168957 | 7851212 | 6946 | TCCAAAATCTYTTCCCAAAAA | 0.075 | 0.075 | 0.252 | 0.483 | 0.716 |
| 29 | 9 | 70186112 | 4610818 | 6947 | TTAGTTTATTKGCTATCAAAC | 0.112 | 0.115 | 0.510 | 0.552 | 0.714 |
| 29 | 9 | 70192722 | 4745004 | 6948 | TAATACCTTGWAACTCTTTTT | 0.920 | 0.899 | 0.860 | 0.860 | 0.909 |
| 29 | 9 | 70200766 | 17053503 | 6949 | CAGTGCAGGAWAGATTTGGGA | 1.278 | 1.251 | 0.947 | 0.832 | 0.734 |
| 29 | 9 | 70207772 | 10491653 | 6950 | TGCAGAAATAYTGTAATTAAT | 0.977 | 0.961 | 1.543 | 0.837 | 0.940 |
| 29 | 9 | 70215494 | 17053650 | 6951 | TTAGATACCGYGGCCTAAAAA | 0.292 | 0.302 | 1.449 | 1.390 | 0.900 |
| 29 | 9 | 70219386 | 4745008 | 6952 | GAATCCGGCTRCTTTAGGAAA | 1.226 | 1.223 | 0.832 | 1.426 | 0.913 |
| 29 | 9 | 70231043 | 10868816 | 6953 | GTGGGAGTTTYGTAGGAACAC | 0.492 | 0.465 | 1.112 | 1.366 | 1.032 |
| 29 | 9 | 70243909 | 11142398 | 6954 | CTTTGTTCAGMTATTTGTGGG | 0.184 | 0.182 | 0.995 | 1.010 | 1.064 |
| 29 | 9 | 70284062 | 17455513 | 6955 | CTGTTCACCTRAAGTTACTTT | 1.568 | 1.612 | 0.869 | 0.848 | 1.062 |
| 29 | 9 | 70295308 | 2184756 | 6956 | TTTTATCCTTYCAGTGTTTCA | 0.107 | 0.108 | 0.707 | 0.960 | 0.843 |
| 29 | 9 | 70300172 | 13299487 | 6957 | TTTGTAGCTTWTACAGCTGTG | 1.030 | 1.010 | 0.960 | 0.672 | 0.902 |
| 29 | 9 | 70317582 | 7864965 | 6958 | TTGCTTGGACRCCTCTCTTCC | 0.058 | 0.059 | 0.509 | 0.528 | 1.310 |
| 29 | 9 | 70326189 | 6560136 | 6959 | AGTGGATTCCYTTCTTTGGAG | 0.653 | 0.690 | 0.539 | 0.475 | 1.389 |
| 29 | 9 | 70333865 | 10868838 | 6960 | TTTATGGCAGWCTCAAAGGCA | 0.590 | 0.632 | 0.152 | 0.440 | 1.458 |
| 29 | 9 | 70339788 | 950840 | 6961 | GAGAGCATGAYGTAATCTTTC | 0.161 | 0.172 | 0.141 | 1.349 | 1.433 |
| 29 | 9 | 70345328 | 7856388 | 6962 | TCTGGAGAAGKTTCAAAGATG | 0.008 | 0.008 | 0.426 | 1.228 | 1.962 |
| 29 | 9 | 70354236 | 17535530 | 6963 | AAAGGGTACCRAGAAATAAGT | 0.040 | 0.041 | 1.548 | 1.885 | 1.845 |
| 29 | 9 | 70360083 | 17535600 | 6964 | TCTATGTACARGTGCCAGAAA | 1.472 | 1.449 | 1.964 | 1.601 | 1.962 |
| 29 | 9 | 70367841 | 7046672 | 6965 | CTACTTTTCCYGAAATCTCTT | 2.612 | 2.942 | 2.930 | 1.841 | 1.734 |
| 29 | 9 | 70376992 | 17535865 | 6966 | TCATGAAAATYGTACCCAAGC | 0.000 | 0.000 | 2.663 | 2.590 | 1.789 |
| 29 | 9 | 70385945 | 1932701 | 6967 | AAGGTTACCTRTGTTTGTTTC | 1.111 | 1.124 | 2.432 | 2.686 | 1.485 |
| 29 | 9 | 70395370 | 7034027 | 6968 | TATCACAGGGRACACTTGAGA | 0.371 | 0.355 | 1.654 | 2.703 | 1.265 |
| 29 | 9 | 70401966 | 17536713 | 6969 | AACCTAAAAASTGCTATGACT | 1.179 | 1.198 | 1.489 | 1.986 | 1.196 |
| 29 | 9 | 70409531 | 2871324 | 6970 | AGAATCCAGAYAATAGGCAGT | 1.480 | 1.618 | 0.990 | 0.922 | 1.314 |
| 29 | 9 | 70416448 | — | 6971 | CATCTTGACTKTTGGCATTTG | 0.321 | 0.318 | 0.971 | 0.769 | 1.478 |
| 29 | 9 | 70422125 | 12686512 | 6972 | TGCTTAAGTAYGACTGCCATA | 0.291 | 0.289 | 0.602 | 0.507 | 2.228 |
| 29 | 9 | 70433166 | 10735599 | 6973 | GAGGCAAGACRTCGTAGGGCA | 0.331 | 0.309 | 0.127 | 0.551 | 1.575 |
| 29 | 9 | 70443198 | 4744606 | 6974 | TTTTCAGCCCMGTTGGGAAGT | 0.311 | 0.301 | 0.077 | 0.403 | 1.763 |
| 29 | 9 | 70450287 | 7865398 | 6975 | ATGAATATTGWTAACTGCCCT | 0.104 | 0.103 | 0.118 | 1.249 | 1.518 |
| 29 | 9 | 70461216 | 11142508 | 6976 | AGAGAGTCCCYTTACACACAA | 0.045 | 0.046 | 0.187 | 1.235 | 1.691 |
| 29 | 9 | 70477224 | 7045775 | 6977 | TTCCTTCTTCYTCCCAAATTC | 0.530 | 0.535 | 2.059 | 1.586 | 1.638 |
| 29 | 9 | 70484986 | 1538669 | 6978 | AATACCCTTGSCCAACCAAAG | 0.653 | 0.625 | 2.452 | 1.675 | 1.338 |
| 29 | 9 | 70497790 | 11142532 | 6979 | ATGGAGAGAAYAGAGGAATGT | 4.337 | 4.091 | 3.308 | 1.912 | 1.271 |
| 29 | 9 | 70509554 | 11142536 | 6980 | TGGCTGACAGYGCCTCAGAAA | 0.104 | 0.106 | 3.004 | 2.084 | 1.228 |
| 29 | 9 | 70515182 | 11142540 | 6981 | GGAATTTTTARAACCCGTTTC | 1.413 | 1.413 | 2.995 | 2.113 | 1.236 |
| 29 | 9 | 70525093 | 1856655 | 6982 | CAACCATGACKCTACAGCAGG | 0.157 | 0.158 | 1.095 | 2.008 | 1.524 |
| 29 | 9 | 70541921 | 4745026 | 6983 | ACATAGAAACRTACATACATT | 1.483 | 1.510 | 1.196 | 1.845 | 1.652 |
| 29 | 9 | 70543565 | 1160742 | 6984 | TAAATGACAARTAGTCTATGT | 0.699 | 0.692 | 0.676 | 0.579 | 1.932 |
| 29 | 9 | 70591948 | 10511986 | 6985 | TGGACTGAAAYTGCAGGCAAA | 0.486 | 0.520 | 0.673 | 1.020 | 2.040 |
| 29 | 9 | 70599241 | 7849151 | 6986 | CAAAAGAAACYTGAAGGATAC | 0.082 | 0.082 | 0.155 | 0.728 | 1.951 |
| 29 | 9 | 70606611 | 11142569 | 6987 | TTGCAGCGTCMTGTCTAATTG | 0.161 | 0.163 | 0.518 | 0.979 | 1.068 |
| 29 | 9 | 70612919 | 17055769 | 6988 | GTGTGTATGCYTGGGTCAGCC | 0.094 | 0.091 | 0.442 | 0.687 | 1.058 |
| 29 | 9 | 70619595 | 1831144 | 6989 | TTAATCCACTRTATCCCTAGC | 1.686 | 1.706 | 0.755 | 0.586 | 0.740 |
| 29 | 9 | 70626414 | 7847799 | 6990 | ACCTGAGAAASCTGAAATGCT | 0.335 | 0.330 | 1.018 | 0.781 | 0.888 |
| 29 | 9 | 70632742 | 11142592 | 6991 | ACCTTAGCTGWTCATAAGCCT | 0.773 | 0.761 | 1.143 | 0.750 | 0.689 |
| 29 | 9 | 70638656 | 1328158 | 6992 | GGGCTTCCTTKCCAGCATAAC | 0.626 | 0.624 | 0.806 | 0.744 | 0.530 |
| 29 | 9 | 70644522 | 10780969 | 6993 | GCAGCATTGARCAAAGTTGAG | 0.376 | 0.374 | 0.665 | 0.948 | 0.560 |
| 29 | 9 | 70650613 | 17463001 | 6994 | CTTCTGCCTGSGATTTAGGAC | 1.049 | 1.049 | 0.409 | 0.593 | 0.563 |
| 29 | 9 | 70657059 | 12552572 | 6995 | GGCCATTCTASCACTGTGTAT | 0.057 | 0.052 | 0.372 | 0.510 | 0.562 |
| 29 | 9 | 70664548 | 560819 | 6996 | AAAAATCCTARTGCCATTTAT | 0.000 | 0.000 | 0.541 | 0.468 | 0.619 |
| 29 | 9 | 70672189 | 1034539 | 6997 | GCTCTGGCCCRGTAGAAATAA | 0.536 | 0.556 | 0.191 | 0.324 | 0.259 |
| 29 | 9 | 70674554 | 10868901 | 6998 | GGGTAAGACCMAAGCAGTCTG | 0.818 | 0.822 | 0.370 | 0.279 | 0.206 |
| 29 | 9 | 70683459 | 1932926 | 6999 | CTGTTTTACCKCCACACCCAA | 0.075 | 0.076 | 0.378 | 0.132 | 0.233 |
| 29 | 9 | 70697458 | 10868911 | 7000 | TTTTCCCATGKGTCAATTTTA | 0.619 | 0.612 | 0.245 | 0.127 | 0.210 |
| 29 | 9 | 70703724 | 10780979 | 7001 | AGACAACTCTRAGTTCCACAG | 0.137 | 0.137 | 0.106 | 0.100 | 0.188 |
| 29 | 9 | 70710180 | 1337024 | 7002 | TTAGGCAAGCRTGTGGCACCG | 0.178 | 0.167 | 0.095 | 0.160 | 0.200 |
| 29 | 9 | 70722520 | 1609625 | 7003 | TACTGACTTGRCTTGAGGCCA | 0.340 | 0.333 | 0.010 | 0.109 | 0.216 |
| 29 | 9 | 70730035 | 11142636 | 7004 | TTTTTACCCARCTAGGTCATT | 0.022 | 0.022 | 0.121 | 0.134 | 0.330 |
| 29 | 9 | 70736650 | 10511992 | 7005 | TTTTGGAAGAYTATATAGAGA | 0.013 | 0.013 | 0.214 | 0.236 | 0.349 |
| 29 | 9 | 70746659 | 4143736 | 7006 | ATCCTTGCAARCAAATGACAT | 0.910 | 0.904 | 0.201 | 0.240 | 0.267 |
| 29 | 9 | 70757996 | 11142642 | 7007 | TCAAAACTACSAGAGGGAAAA | 0.473 | 0.455 | 0.620 | 0.385 | 0.265 |
| 29 | 9 | 70765970 | 3010419 | 7008 | TAGTCCAGGARACAGAATATT | 0.257 | 0.253 | 0.692 | 0.473 | 0.207 |
| 29 | 9 | 70775011 | 2993024 | 7009 | TAAGTCACAGMATCTTAGTAT | 1.113 | 1.139 | 0.579 | 0.553 | 0.253 |
| 29 | 9 | 70782222 | 17056028 | 7010 | CTCCTTTTTCWAACAAAGCTC | 0.161 | 0.158 | 0.669 | 0.573 | 0.344 |
| 29 | 9 | 70788186 | 12351255 | 7011 | TCAAACTCTCYGATGCTGAAT | 0.736 | 0.705 | 0.699 | 0.387 | 0.358 |
| 29 | 9 | 70794271 | 3010423 | 7012 | TGAGTTAGTAMAAACTTGGTG | 0.641 | 0.653 | 0.286 | 0.392 | 0.364 |
| 29 | 9 | 70800561 | 7022875 | 7013 | GGGTGCCCGAYTGAGAATCAC | 0.326 | 0.323 | 0.316 | 0.529 | 0.416 |
| 29 | 9 | 70808080 | 1337022 | 7014 | TGCGCTTTTTSCAAGTGTGCT | 0.096 | 0.099 | 0.233 | 0.327 | 0.376 |
| 29 | 9 | 70814967 | — | 7015 | ATGGGCCTTCYGTGGGGAGTA | 0.253 | 0.243 | 0.269 | 0.301 | 0.378 |
| 29 | 9 | 70821303 | 2909300 | 7016 | TAGTGACTGCYTCAAAAGTGG | 0.448 | 0.442 | 0.291 | 0.215 | 0.494 |
| 29 | 9 | 70827862 | 1558924 | 7017 | AAGTGCCTACYAGATATACAG | 0.773 | 0.724 | 0.290 | 0.226 | 0.520 |
| 29 | 9 | 70839607 | 17056164 | 7018 | TCAGCATTTTMTTGCCTTACA | 0.378 | 0.384 | 0.311 | 0.255 | 0.673 |
| 29 | 9 | 70846825 | 10868940 | 7019 | TGGAGCCAGCRGTTTTAGCTT | 0.037 | 0.037 | 0.377 | 0.448 | 0.711 |
| 29 | 9 | 70874370 | 11142672 | 7020 | TGGACTGCTCRTATTTGAGTC | 0.297 | 0.306 | 0.296 | 0.715 | 0.599 |
| 29 | 9 | 70882519 | 573250 | 7021 | ATTTTAATGAWCCTACTGAAT | 0.677 | 0.700 | 0.483 | 0.840 | 0.678 |
| 29 | 9 | 70890955 | 625699 | 7022 | GCCCATAAAGYATCAAGGGAA | 0.481 | 0.488 | 0.920 | 0.848 | 0.677 |
| 29 | 9 | 70911110 | 659797 | 7023 | ATAATTAACARCTCTGAGTTT | 0.934 | 0.894 | 1.041 | 0.768 | 0.691 |
| 29 | 9 | 70919059 | 661604 | 7024 | TTAACACCCTMCTGCTTCAGG | 1.266 | 1.262 | 1.055 | 0.911 | 0.785 |
| 29 | 9 | 70925435 | 10868946 | 7025 | TTCTGGAATCMACTGTGTACC | 0.926 | 0.970 | 0.983 | 0.897 | 0.682 |
| 29 | 9 | 70931529 | 505676 | 7026 | AAAATTTTTGRACACCATTGC | 0.923 | 0.919 | 0.982 | 0.828 | 0.621 |
| 29 | 9 | 70932502 | 656875 | 7027 | ATGAGTCCCCRTTGTGCTTGA | 0.046 | 0.047 | 0.623 | 1.004 | 0.637 |
| 29 | 9 | 70948361 | 1329748 | 7028 | GAAATAATGGRGCAGATGATC | 0.689 | 0.665 | 0.362 | 0.819 | 0.609 |
| 29 | 9 | 70953863 | 10868950 | 7029 | CTGAGTCTAGYTTATTACAAA | 0.216 | 0.217 | 0.371 | 0.474 | 0.521 |
| 29 | 9 | 70960548 | 17521958 | 7030 | AAATTCTGCCYAACAATACAT | 0.315 | 0.308 | 0.449 | 0.288 | 0.535 |
| 30 | 9 | 93175798 | 2927567 | 7031 | ATGGAAACTAMCAACTACGTC | 0.775 | 0.750 | 1.705 | 2.027 | 1.799 |
| 30 | 9 | 93181986 | 2995992 | 7032 | ACCAGCATGTKTGTAGTCACA | 0.272 | 0.276 | 1.938 | 2.108 | 1.961 |
| 30 | 9 | 93206088 | 4744229 | 7033 | ATCGGGTGGCMCTATGACTGC | 1.418 | 1.377 | 1.690 | 1.769 | 1.691 |
| 30 | 9 | 93215235 | 11789033 | 7034 | AAGGACTTTAYGCATTTGCAC | 1.244 | 1.218 | 1.573 | 1.684 | 1.706 |
| 30 | 9 | 93221289 | 7021710 | 7035 | TGGAAGCACTYCACCTCTCAG | 0.976 | 0.930 | 1.690 | 1.501 | 1.677 |
| 30 | 9 | 93242815 | 7020852 | 7036 | GGTCCTACACYCCAAAAAGTT | 0.855 | 0.868 | 1.279 | 1.218 | 1.409 |
| 30 | 9 | 93245937 | 4744239 | 7037 | GGAACGTCCAYGAATGGAGAG | 0.250 | 0.262 | 1.039 | 1.449 | 1.324 |
| 30 | 9 | 93268664 | 7859267 | 7038 | TTGTCCTCTGKTTTCCTATTA | 0.559 | 0.573 | 0.511 | 1.097 | 1.286 |
| 30 | 9 | 93295671 | 1331588 | 7039 | GAGCGTAAAARTGTTCATAGT | 0.854 | 0.847 | 0.598 | 0.643 | 1.051 |
| 30 | 9 | 93308995 | 10512224 | 7040 | CAAATGCTGAKTCATCTTGTT | 0.000 | 0.001 | 0.629 | 0.541 | 0.991 |
| 30 | 9 | 93322209 | 4744245 | 7041 | TGGGATTCCTYGCTGCAGTCC | 1.024 | 1.063 | 0.389 | 0.643 | 0.772 |
| 30 | 9 | 93324842 | 11791374 | 7042 | GCATTTACCAYGTAACCTTGC | 0.361 | 0.362 | 0.326 | 0.582 | 0.614 |
| 30 | 9 | 93336138 | 1556416 | 7043 | GACATTTGAARGATGCACCTA | 0.012 | 0.011 | 0.788 | 0.501 | 0.457 |
| 30 | 9 | 93345702 | 16909211 | 7044 | TTTAAAGTAARGAGCTCGTTG | 0.642 | 0.644 | 0.439 | 0.406 | 0.352 |
| 30 | 9 | 93359002 | 4743928 | 7045 | GACCCAGGGCMGAGCTTACTG | 1.211 | 1.176 | 0.393 | 0.495 | 0.527 |
| 30 | 9 | 93368824 | 10739950 | 7046 | ATCACCTACCYTTTTGAAGCA | 0.111 | 0.107 | 0.524 | 0.311 | 0.586 |
| 30 | 9 | 93377480 | 10821152 | 7047 | TATAGGGAGCYGATAAAATTG | 0.242 | 0.235 | 0.432 | 0.253 | 0.514 |
| 30 | 9 | 93386292 | 10821158 | 7048 | ATACTGTTGAYGTTTTGAAGT | 0.420 | 0.395 | 0.138 | 0.541 | 1.219 |
| 30 | 9 | 93388594 | 928367 | 7049 | CCATGCTCCARTAACCTTCTC | 0.431 | 0.421 | 0.136 | 0.596 | 1.581 |
| 30 | 9 | 93422615 | 10821163 | 7050 | TATTCTGTACSGAGTTCAGAG | 0.097 | 0.098 | 0.470 | 0.423 | 1.792 |
| 30 | 9 | 93427445 | 1000852 | 7051 | CAAACATGAGRATCTGATTGG | 0.186 | 0.191 | 0.654 | 1.597 | 2.248 |
| 30 | 9 | 93437856 | 10821168 | 7052 | GGCTTACCCAYGGTCTGTCCT | 1.322 | 1.244 | 0.661 | 2.827 | 2.304 |
| 30 | 9 | 93446202 | 2989751 | 7053 | GACGGAGAGCRAGACTTCTTT | 0.893 | 0.878 | 2.824 | 3.149 | 2.088 |
| 30 | 9 | 93459352 | 12551314 | 7054 | TCCCAAGAGGMAATTGTGGGG | 0.418 | 0.431 | 3.676 | 2.959 | 2.513 |
| 30 | 9 | 93475886 | 1806458 | 7055 | GGTGTTCCTCRCAGGTGGTGA | 4.364 | 4.488 | 3.723 | 3.076 | 2.853 |
| 30 | 9 | 93485419 | 6479499 | 7056 | AGCCCTCTAGWTCCTTGAAGT | 3.119 | 3.149 | 3.380 | 2.972 | 2.688 |
| 30 | 9 | 93491981 | — | 7057 | ACCAACACGGYACACTCACAC | 0.000 | 0.000 | 4.568 | 3.198 | 2.674 |
| 30 | 9 | 93498640 | 10992831 | 7058 | CTTCCGATTGRACTGTCCCTT | 1.279 | 1.214 | 2.171 | 3.428 | 2.942 |
| 30 | 9 | 93527276 | 7024542 | 7059 | TACCTTCCAGYGCCATCAGGA | 0.921 | 0.880 | 1.487 | 3.454 | 2.926 |
| 30 | 9 | 93550895 | 7874616 | 7060 | CAAATGGGGAYTGTGGAAGAG | 1.056 | 0.999 | 1.529 | 2.252 | 2.885 |
| 30 | 9 | 93563775 | 2398871 | 7061 | CTACACGAGAYAACTGACATA | 1.156 | 1.158 | 1.032 | 1.524 | 3.117 |
| 30 | 9 | 93569564 | 16909421 | 7062 | AGGATCTGACMTTTTGAGTCA | 0.642 | 0.636 | 1.145 | 1.426 | 3.163 |
| 30 | 9 | 93590628 | 4392963 | 7063 | AGCAGCTCTTMCCCTGTGGAT | 0.003 | 0.003 | 1.029 | 1.017 | 1.937 |
| 30 | 9 | 93619309 | 2263356 | 7064 | GAAAATCACAYGTGTTGTAAA | 0.973 | 1.002 | 0.539 | 1.100 | 1.239 |
| 30 | 9 | 93634162 | 7030496 | 7065 | TTTTTGATTGYTGATTAAATC | 0.759 | 0.754 | 0.293 | 0.683 | 1.060 |
| 30 | 9 | 93657887 | 12003661 | 7066 | AGGGGGATTASTTTGCCAAGG | 0.280 | 0.238 | 0.788 | 0.335 | 0.719 |
| 30 | 9 | 93667688 | 3957506 | 7067 | GGGACCGACCYGAGGATGAAG | 0.066 | 0.067 | 0.379 | 0.376 | 0.887 |
| 30 | 9 | 93699974 | 3209003 | 7068 | CCATACCTTGKGTGTTTGTAT | 1.035 | 1.042 | 0.161 | 0.398 | 0.949 |
| 30 | 9 | 93706342 | 12337678 | 7069 | CTGGACCTGCYTGTGGTTCTT | 0.059 | 0.060 | 0.316 | 0.200 | 1.014 |
| 30 | 9 | 93714065 | 16909534 | 7070 | ATGGGGGTAGWTTGTAATGAG | 0.109 | 0.114 | 0.293 | 0.352 | 0.847 |
| 30 | 9 | 93729092 | 10761299 | 7071 | TGTGTGGAGGYCACTGCACTA | 0.782 | 0.774 | 0.065 | 0.672 | 1.303 |
| 30 | 9 | 93747094 | 17625535 | 7072 | CAAAAACATCWACGGAAAGGG | 0.009 | 0.009 | 0.494 | 1.249 | 1.067 |
| 30 | 9 | 93756862 | 10733751 | 7073 | CAGAACAAGGSCCACTGCAAG | 0.208 | 0.207 | 1.056 | 0.829 | 1.025 |
| 30 | 9 | 93764675 | 10992984 | 7074 | AGCCTAGAATRTGACCTCTTG | 1.383 | 1.364 | 1.347 | 1.427 | 1.154 |
| 31 | 9 | 110265356 | 7031072 | 7075 | GAGTGCTGACRGAAGTTAATG | 1.210 | 1.251 | 1.278 | 0.778 | 0.577 |
| 31 | 9 | 110272750 | 4978426 | 7076 | ATCTACAGATKCAGGGTCGGT | 0.668 | 0.649 | 0.978 | 1.096 | 0.571 |
| 31 | 9 | 110283987 | 16915037 | 7077 | TCCTTGGGTAMAGGCTTTCAT | 0.575 | 0.548 | 0.763 | 1.268 | 0.589 |
| 31 | 9 | 110293281 | 4421419 | 7078 | AACTTGACAGRACTTTAAGAC | 0.298 | 0.303 | 0.631 | 0.978 | 0.621 |
| 31 | 9 | 110299334 | 7875113 | 7079 | TTGCTGCCTGMATAACACTGG | 0.386 | 0.382 | 0.681 | 0.784 | 0.614 |
| 31 | 9 | 110314062 | 10817025 | 7080 | CCGTGAGGACYTGGCATTGCC | 0.900 | 0.956 | 0.465 | 0.405 | 0.700 |
| 31 | 9 | 110323375 | 3818766 | 7081 | CCTGTCTATCRCTATTTCTAG | 0.704 | 0.726 | 0.469 | 0.288 | 0.645 |
| 31 | 9 | 110350156 | 4427239 | 7082 | CGTGCTTCCTRTTAGTCTCCA | 0.104 | 0.106 | 0.379 | 0.169 | 0.517 |
| 31 | 9 | 110358254 | 17734141 | 7083 | TGAATGAAGGRAACACTTAAA | 0.302 | 0.303 | 0.158 | 0.249 | 0.315 |
| 31 | 9 | 110364426 | 2184245 | 7084 | GCTGAATGTCYGCCTGTCCTA | 0.152 | 0.156 | 0.025 | 0.349 | 0.211 |
| 31 | 9 | 110375601 | 4300064 | 7085 | ACATTGGCCAYCATCTAAACT | 0.200 | 0.205 | 0.124 | 0.227 | 0.247 |
| 31 | 9 | 110382596 | 10817036 | 7086 | CCTCTAACAAWGACTGGTATT | 0.049 | 0.048 | 0.262 | 0.129 | 0.215 |
| 31 | 9 | 110389092 | 10817039 | 7087 | AGACTGTGGTRTGGGTAAAAG | 0.646 | 0.661 | 0.321 | 0.110 | 0.220 |
| 31 | 9 | 110399504 | 16915129 | 7088 | TATTCAGTCAYAGTGTTTCCA | 0.730 | 0.741 | 0.301 | 0.204 | 0.113 |
| 31 | 9 | 110425355 | 16915134 | 7089 | TCTTTCTGCCRTGAATTTAAC | 0.378 | 0.375 | 0.306 | 0.182 | 0.059 |
| 31 | 9 | 110444019 | 12236915 | 7090 | CCTAGGGACCRGTTCCTGAAT | 0.135 | 0.137 | 0.360 | 0.213 | 0.130 |
| 31 | 9 | 110451178 | 4295728 | 7091 | GTTGTGCAATYTTTTTAACGT | 0.086 | 0.084 | 0.145 | 0.226 | 0.155 |
| 31 | 9 | 110457541 | 10980489 | 7092 | CAGGTTTTTGYTCCCTTTTCT | 0.789 | 0.796 | 0.131 | 0.133 | 0.307 |
| 31 | 9 | 110463553 | 10980495 | 7093 | TCCAGCAATTYCTACTCTAAC | 0.041 | 0.041 | 0.117 | 0.153 | 0.423 |
| 31 | 9 | 110474247 | 10521102 | 7094 | GTATTTCTGCRTAGGTAGAAT | 0.342 | 0.342 | 0.140 | 0.165 | 0.598 |
| 31 | 9 | 110483333 | 3001114 | 7095 | GATCCACTCAYGGGTTACACA | 0.115 | 0.120 | 0.143 | 0.394 | 0.735 |
| 31 | 9 | 110491782 | 3001153 | 7096 | TACATGTCTCMTCTGCCCTGT | 0.136 | 0.140 | 0.297 | 0.608 | 0.791 |
| 31 | 9 | 110501698 | 3010804 | 7097 | ACAGGCTATGMAGTGTGGAGT | 0.799 | 0.806 | 0.620 | 0.676 | 0.805 |
| 31 | 9 | 110511161 | 3001125 | 7098 | TGGATGATGAMAACCTTTTCG | 0.454 | 0.441 | 0.909 | 1.105 | 1.120 |
| 31 | 9 | 110541167 | 16915263 | 7099 | AGATGATTTTYGCACACATTT | 1.254 | 1.217 | 1.414 | 1.323 | 1.201 |
| 31 | 9 | 110548452 | 4401940 | 7100 | GGAATTTTCARTGAGGGTTTG | 0.887 | 0.924 | 1.968 | 1.446 | 1.083 |
| 31 | 9 | 110557083 | 10759461 | 7101 | CCCAGTTATGMGTTAGCTGCT | 0.973 | 0.973 | 2.063 | 2.150 | 2.504 |
| 31 | 9 | 110570331 | 10817087 | 7102 | TGTCATTTAAYAGTTTGCTTT | 1.284 | 1.292 | 1.445 | 1.778 | 2.681 |
| 31 | 9 | 110586553 | 4144418 | 7103 | ACAGCTAACTYAGAAAGACAG | 1.085 | 1.050 | 1.643 | 1.755 | 2.857 |
| 31 | 9 | 110597275 | — | 7104 | CAATTTAAACYTGACAGGTAC | 0.391 | 0.386 | 1.403 | 3.271 | 2.824 |
| 31 | 9 | 110603895 | 1013820 | 7105 | GGCCTCCTAGMAGAAGCGGAG | 1.187 | 1.160 | 0.816 | 2.695 | 2.375 |
| 31 | 9 | 110612005 | 10980564 | 7106 | TACGAATTTCRCAGGTTTTCA | 0.357 | 0.362 | 2.903 | 3.206 | 2.612 |
| 31 | 9 | 110625482 | — | 7107 | TTTTAATTCTRTCATTTGTGT | 0.223 | 0.219 | 2.857 | 2.252 | 2.157 |
| 31 | 9 | 110648651 | 1409686 | 7108 | TGCTCTGGTTMGTATTGGTAG | 4.295 | 4.153 | 3.060 | 1.934 | 2.134 |
| 31 | 9 | 110654946 | 1180281 | 7109 | ATGGCATCATRCTAACAGACA | 0.415 | 0.408 | 3.054 | 2.025 | 1.987 |
| 31 | 9 | 110663567 | 2767001 | 7110 | CAGTGAGAGGRATCAAATGAG | 0.446 | 0.405 | 2.496 | 1.600 | 1.649 |
| 31 | 9 | 110673638 | 12555499 | 7111 | TGGATTGCCTRTGATACTTCT | 0.056 | 0.043 | 0.161 | 2.049 | 1.402 |
| 31 | 9 | 110691415 | 526584 | 7112 | AAACTGAATGYTCATACATGT | 0.226 | 0.229 | 0.111 | 1.759 | 1.434 |
| 31 | 9 | 110692760 | — | 7113 | ACAGGGCCTCRAATTCTTCCC | 0.520 | 0.512 | 0.247 | 0.319 | 1.111 |
| 31 | 9 | 110715246 | 6833 | 7114 | GTATAAAGTAYGTGCAAACAC | 0.224 | 0.226 | 0.249 | 0.327 | 1.014 |
| 31 | 9 | 110737318 | 7851666 | 7115 | TCTTCTTAGCYGGGAAACTAG | 0.872 | 0.896 | 0.562 | 0.300 | 0.939 |
| 31 | 9 | 110743076 | — | 7116 | AGGAGAAGTCRTGGCCTAAAG | 0.017 | 0.017 | 0.538 | 0.298 | 0.289 |
| 31 | 9 | 110746832 | 3780528 | 7117 | TACTGGAGCAYGGACCATTTC | 1.044 | 0.995 | 0.565 | 0.278 | 0.312 |
| 31 | 9 | 110761555 | 2805386 | 7118 | CATATCCAAARTGCTTCGCTG | 0.389 | 0.392 | 0.261 | 0.213 | 0.346 |
| 31 | 9 | 110769583 | 2254841 | 7119 | ATAGTAGATGRTTTAGTTACT | 0.354 | 0.357 | 0.279 | 0.467 | 0.479 |
| 31 | 9 | 110779435 | 6477795 | 7120 | ATTTATGCCARATAAGTATTT | 0.018 | 0.018 | 0.059 | 0.380 | 0.494 |
| 31 | 9 | 110786780 | 7866715 | 7121 | GGATAAGAACKCTATATTTCA | 0.129 | 0.127 | 0.315 | 0.561 | 0.452 |
| 31 | 9 | 110806340 | 10817125 | 7122 | GCTAGGGATCYTAAAATACTT | 0.120 | 0.117 | 0.397 | 0.471 | 0.434 |
| 31 | 9 | 110812487 | 10817126 | 7123 | GGGAAAAATGYCCCTTGTTCT | 1.397 | 1.309 | 0.647 | 0.453 | 0.280 |
| 31 | 9 | 110819193 | 4366150 | 7124 | CAGAAAACACRTGGGAAAAAG | 0.529 | 0.521 | 0.885 | 0.437 | 0.453 |
| 31 | 9 | 110825551 | 2192591 | 7125 | ATGATAAGTGMAAAACAGGTT | 0.632 | 0.662 | 0.996 | 0.454 | 0.271 |
| 31 | 9 | 110832787 | 10817128 | 7126 | GTAGTATAGCMTTTTCTCCAC | 0.704 | 0.646 | 0.492 | 0.428 | 0.476 |
| 31 | 9 | 110843884 | 2192593 | 7127 | GAACAAGATTWAGCTTTTAAC | 0.313 | 0.309 | 0.341 | 0.686 | 0.436 |
| 31 | 9 | 110852489 | 10759480 | 7128 | TCAGACAATTWTTTTTGGTAC | 0.320 | 0.309 | 0.142 | 0.299 | 0.494 |
| 31 | 9 | 110859197 | — | 7129 | TCGAGTCTGASTTTTTTGAAG | 0.130 | 0.131 | 0.200 | 0.553 | 0.587 |
| 31 | 9 | 110862610 | — | 7130 | AGATCAGGAGSCTGGAGTCCT | 0.080 | 0.081 | 0.145 | 0.410 | 0.838 |
| 31 | 9 | 110871972 | 6477803 | 7131 | TGAAACTAAAYTGACAGCGTC | 0.952 | 0.902 | 0.557 | 0.314 | 0.857 |
| 31 | 9 | 110882740 | 10980705 | 7132 | TCCATAAAACYTAGCTCATTA | 0.078 | 0.079 | 0.580 | 0.381 | 0.738 |
| 32 | 10 | 19899999 | 3852478 | 7133 | TTTGAGTCTTRTGTTTTTCCA | 0.064 | 0.066 | 0.695 | 0.337 | 0.347 |
| 32 | 10 | 19905763 | 11010520 | 7134 | ATCCTCCCCAYAGGAAACCAC | 1.920 | 2.100 | 0.609 | 0.374 | 0.225 |
| 32 | 10 | 19916301 | 12249318 | 7135 | ATATCACTGCRTATGACAATG | 0.370 | 0.373 | 0.845 | 0.623 | 0.326 |
| 32 | 10 | 19924660 | 11010622 | 7136 | GGGAGATGCCRTTATGTGTTC | 0.188 | 0.189 | 0.919 | 0.588 | 0.478 |
| 32 | 10 | 19935038 | 3844359 | 7137 | CATTTTCAGARTTAATTGTGT | 0.640 | 0.642 | 0.399 | 0.607 | 0.586 |
| 32 | 10 | 19942794 | 3904905 | 7138 | GATCTTTATGYTCACCTTTAC | 0.197 | 0.189 | 0.349 | 0.956 | 0.688 |
| 32 | 10 | 19951897 | 12261476 | 7139 | ATTTGGAAAARGGTAAAATTA | 0.876 | 0.847 | 0.379 | 0.547 | 0.606 |
| 32 | 10 | 19958526 | 12219684 | 7140 | CCAAGCTTCAYGGGGTAGCCG | 0.228 | 0.240 | 0.497 | 0.592 | 1.002 |
| 32 | 10 | 19964003 | 10827695 | 7141 | TTGGATCAAAYCTTTTATAGG | 0.233 | 0.245 | 0.785 | 0.657 | 1.534 |
| 32 | 10 | 19976791 | 7474617 | 7142 | ACATGACACARCTTTTTTCCA | 0.937 | 0.949 | 0.635 | 0.481 | 1.417 |
| 32 | 10 | 19986172 | — | 7143 | TTTTGCTTTGYGGACTAATCT | 0.867 | 0.807 | 0.714 | 0.957 | 1.484 |
| 32 | 10 | 19994710 | — | 7441 | TTGCTCATCTRAATTAATCCT | 0.515 | 0.519 | 0.604 | 1.247 | 1.559 |
| 32 | 10 | 20004919 | 7916152 | 7145 | TATCCTTAAARGTAAAGATGA | 0.410 | 0.415 | 1.055 | 1.795 | 1.359 |
| 32 | 10 | 20014936 | 4748608 | 7146 | TATCTGAGTCRTGCAGCACCT | 0.047 | 0.047 | 1.485 | 2.083 | 1.581 |
| 32 | 10 | 20019440 | 16919291 | 7147 | CTCTAAATGGRTGACGAGGTT | 2.013 | 1.993 | 1.911 | 1.871 | 1.583 |
| 32 | 10 | 20031847 | 12256382 | 7148 | TTGAAGTAAAMTTTTGTATTG | 1.813 | 1.773 | 2.181 | 1.514 | 1.828 |
| 32 | 10 | 20042742 | 2025789 | 7149 | AGTCTGGTTCYGGAGTTAGCA | 1.610 | 1.603 | 2.324 | 1.735 | 1.943 |
| 32 | 10 | 20043985 | 2478746 | 7150 | AAATATGTACRTGCACTCTTT | 1.168 | 1.178 | 1.652 | 2.041 | 1.836 |
| 32 | 10 | 20050326 | 16919309 | 7151 | CAACTCAGATYCACCATTAAT | 0.441 | 0.452 | 1.471 | 2.369 | 1.773 |
| 32 | 10 | 20057858 | 7100283 | 7152 | ACACAACTTAMTCAAATTAAT | 0.155 | 0.157 | 1.320 | 1.910 | 1.667 |
| 32 | 10 | 20069776 | 7073601 | 7153 | TGGTCTGGCTYTTTTAGAGGT | 1.354 | 1.304 | 1.220 | 1.548 | 1.741 |
| 32 | 10 | 20106875 | 4748610 | 7154 | CTCAGTGCTTYGATATAGGAG | 1.144 | 1.210 | 0.936 | 1.180 | 1.887 |
| 32 | 10 | 20119156 | 12763948 | 7155 | GGTCTTTCAAYTGAAACTAGA | 0.740 | 0.732 | 1.132 | 0.702 | 1.467 |
| 32 | 10 | 20125405 | 12263244 | 7156 | CCTATCCCCARTTTGATACTA | 0.018 | 0.018 | 0.709 | 0.617 | 0.950 |
| 32 | 10 | 20131169 | 16919362 | 7157 | CAGCAGTCAGYATTGAATGGT | 0.444 | 0.450 | 0.252 | 0.801 | 0.667 |
| 32 | 10 | 20140825 | 11011581 | 7158 | CACTCTGCATYGTACAGCATA | 0.544 | 0.539 | 0.152 | 0.440 | 0.618 |
| 32 | 10 | 20150341 | 11011636 | 7159 | GACGTGTTGTYTTAGAATTCT | 0.083 | 0.085 | 0.382 | 0.199 | 0.516 |
| 32 | 10 | 20159308 | 12263862 | 7160 | TTCAGGTCAGYATCTACCATG | 0.292 | 0.296 | 0.283 | 0.147 | 0.727 |
| 32 | 10 | 20170064 | 333693 | 7161 | TCCTTCTCTTYAAATCTTACA | 0.781 | 0.746 | 0.146 | 0.345 | 0.581 |
| 32 | 10 | 20176365 | 7900249 | 7162 | GTCATTATCTSTGGTGGAAAA | 0.135 | 0.141 | 0.237 | 0.249 | 0.657 |
| 32 | 10 | 20182706 | 16919431 | 7163 | GGTGCTTGGTKCCACAGGTTG | 0.147 | 0.147 | 0.484 | 0.361 | 0.631 |
| 32 | 10 | 20189724 | 7895714 | 7164 | TCATTCTCTTWTGCGTTGCAA | 0.455 | 0.463 | 0.211 | 0.553 | 1.075 |
| 32 | 10 | 20196780 | 17756750 | 7165 | GTGTGTATGTRTAAATGTACC | 0.970 | 0.924 | 0.570 | 0.975 | 1.165 |
| 32 | 10 | 20203412 | 752319 | 7166 | AATCTTGTTCRTTGTAAAAGA | 0.019 | 0.020 | 0.837 | 0.923 | 1.131 |
| 32 | 10 | 20209606 | 11592226 | 7167 | GTATTCAGTAYTTTCCATGAG | 1.048 | 1.045 | 1.219 | 1.404 | 1.096 |
| 32 | 10 | 20216969 | 11011658 | 7168 | ACACGCCTATYACTCCACTTC | 0.716 | 0.704 | 1.178 | 1.634 | 1.105 |
| 32 | 10 | 20222872 | 17757908 | 7169 | TTAGGGGGTCRACCATGCTAA | 1.482 | 1.494 | 2.045 | 1.553 | 0.898 |
| 32 | 10 | 20234345 | 12774164 | 7170 | TATTATTTCTSCTAACTGGGT | 0.587 | 0.620 | 1.962 | 1.298 | 1.037 |
| 32 | 10 | 20241326 | 965787 | 7171 | TAACAAAATGRGCTAGAACTT | 1.561 | 1.522 | 1.645 | 1.396 | 1.125 |
| 32 | 10 | 20249001 | 1887035 | 7172 | GTATTTGCTAYCACTTTACCA | 1.012 | 0.984 | 0.908 | 1.076 | 1.142 |
| 32 | 10 | 20256253 | 17758689 | 7173 | CCGGAGGAAGYTGACATTTCA | 0.279 | 0.278 | 0.742 | 1.041 | 1.055 |
| 32 | 10 | 20263456 | 9651367 | 7174 | TGCGATCATTRCATTGGTCAC | 0.023 | 0.023 | 0.158 | 0.677 | 1.189 |
| 32 | 10 | 20276277 | 7898627 | 7175 | TGGGAAACACRCCACTGTTGA | 0.189 | 0.195 | 0.095 | 0.691 | 1.225 |
| 32 | 10 | 20283648 | 1926204 | 7176 | AGATTGCTCAYATAGAGAACA | 0.101 | 0.093 | 0.189 | 0.397 | 1.019 |
| 32 | 10 | 20290990 | 1409341 | 7177 | AGTGACGATAYTTATTTGTGT | 0.737 | 0.756 | 0.422 | 0.284 | 1.101 |
| 32 | 10 | 20298993 | 7922844 | 7178 | CTCATGCCTAYGTTCTTGAAC | 0.564 | 0.551 | 0.518 | 0.506 | 1.035 |
| 32 | 10 | 20313589 | 1111367 | 7179 | GGACCTCAGAYAGTATAAGTT | 0.691 | 0.671 | 0.728 | 0.501 | 0.721 |
| 32 | 10 | 20317089 | 4311960 | 7180 | ACTATCCTTTYGTTAGAATTT | 0.478 | 0.472 | 0.860 | 1.062 | 0.668 |
| 32 | 10 | 20331025 | 957210 | 7181 | CAATCTAGAAMTTTTGGATTT | 0.561 | 0.545 | 0.650 | 1.192 | 0.629 |
| 32 | 10 | 20340113 | — | 7182 | TATGTTGAACRAAGGGCTCAG | 1.078 | 1.074 | 1.323 | 1.005 | 2.177 |
| 32 | 10 | 20345947 | 10764184 | 7183 | TACTTAGCTARTTTCCTCTCT | 0.019 | 0.019 | 1.239 | 1.170 | 2.345 |
| 32 | 10 | 20351919 | 4311961 | 7184 | GAGACAAACAYTGTGGCAGTG | 1.821 | 1.776 | 1.064 | 0.894 | 2.459 |
| 32 | 10 | 20359898 | 10740960 | 7185 | AAATGAGACARCAAATGGAGA | 0.289 | 0.282 | 0.895 | 2.936 | 2.216 |
| 32 | 10 | 20366165 | 17688735 | 7186 | TAGAAATCTCRTACATATTCT | 0.394 | 0.379 | 0.895 | 2.660 | 1.923 |
| 32 | 10 | 20376501 | 2358915 | 7187 | CATTATTTCCWAAGTGTGTCT | 0.793 | 0.796 | 2.557 | 2.224 | 1.598 |
| 32 | 10 | 20387770 | 9664354 | 7188 | AGTTCTGTCARTGGAAAATTT | 0.185 | 0.184 | 2.623 | 2.062 | 1.628 |
| 32 | 10 | 20406431 | 12250505 | 7189 | ATGGGGTTACRATGGGGTTAG | 4.736 | 4.267 | 2.643 | 1.439 | 1.725 |
| 32 | 10 | 20413892 | 7917917 | 7190 | TTTTAATATGRTCTCAGTGGC | 0.563 | 0.533 | 2.205 | 1.295 | 1.600 |
| 32 | 10 | 20419570 | 11011792 | 7191 | GTAGAAAAAARTTACAGAAAA | 0.298 | 0.284 | 2.029 | 1.429 | 1.789 |
| 32 | 10 | 20430647 | 2358839 | 7192 | TGTGGGATTAKGTTCTCCTGA | 0.268 | 0.272 | 0.088 | 1.553 | 1.484 |
| 32 | 10 | 20438674 | 2460593 | 7193 | CCATTGCAATRAATCTTTAGA | 0.004 | 0.004 | 0.146 | 1.823 | 1.381 |
| 32 | 10 | 20445668 | 2461939 | 7194 | AATTTGAAGTYAGAATTGTTG | 0.059 | 0.058 | 0.389 | 0.682 | 1.426 |
| 32 | 10 | 20454519 | 2884565 | 7195 | AGGTTTGAGCSTGACCATAGG | 0.797 | 0.837 | 0.723 | 0.676 | 1.347 |
| 32 | 10 | 20464632 | 16919903 | 7196 | TATATTGGTAMTTTTATGAAC | 1.030 | 1.064 | 1.148 | 0.622 | 1.339 |
| 32 | 10 | 20471634 | 12217582 | 7197 | CTGCAAACATRTAGACTTTAT | 1.121 | 1.087 | 1.491 | 0.715 | 0.476 |
| 32 | 10 | 20477435 | 16919952 | 7198 | CAAACTGACARAGTAAAGAAT | 0.961 | 0.940 | 1.060 | 0.819 | 0.459 |
| 32 | 10 | 20483092 | 7895366 | 7199 | CAGCTTCTTCRTATTAAGTTG | 0.558 | 0.533 | 0.803 | 0.832 | 0.554 |
| 32 | 10 | 20495937 | 2358856 | 7200 | CAAACGTAGARTTCAGAGAAA | 0.014 | 0.015 | 0.485 | 0.730 | 0.509 |
| 32 | 10 | 20504910 | 11593311 | 7201 | AAACCCAGGCRAGCACATTAG | 0.539 | 0.557 | 0.201 | 0.547 | 0.770 |
| 32 | 10 | 20515216 | 2358871 | 7202 | AATTGAAGGCRTCAAAGAGAG | 0.377 | 0.366 | 0.121 | 0.427 | 1.501 |
| 32 | 10 | 20522577 | 17699919 | 7203 | GGATAAAAGGRGTGTGCTCTG | 0.090 | 0.089 | 0.228 | 0.199 | 1.702 |
| 32 | 10 | 20528200 | 10508617 | 7204 | GAGACAGAGGWTACTTACTAC | 0.000 | 0.000 | 0.307 | 0.313 | 1.529 |
| 32 | 10 | 20536276 | 11011870 | 7205 | CTGGGATTAAYTTAATAATTT | 0.000 | 0.000 | 0.190 | 1.242 | 1.299 |
| 32 | 10 | 20543273 | 16920111 | 7206 | GACCTAAATAKGTGATGTTTT | 0.700 | 0.701 | 0.707 | 1.605 | 1.122 |
| 32 | 10 | 20548945 | 6482103 | 7207 | CCTTTTTAGGMCATTGTTTAT | 0.090 | 0.090 | 1.680 | 1.653 | 1.190 |
| 32 | 10 | 20555548 | 11597607 | 7208 | CATCAGGATAYACTTTTTCCC | 1.117 | 1.012 | 1.970 | 1.694 | 1.217 |
| 32 | 10 | 20562077 | 1930227 | 7209 | TGTATAGTTGSACGAGTTGTA | 2.464 | 2.496 | 1.756 | 1.557 | 1.141 |
| 32 | 10 | 20564148 | 10827998 | 7210 | TGTCCTTGCTYTCACTCAGCA | 1.410 | 1.407 | 1.917 | 1.482 | 1.102 |
| 32 | 10 | 20574551 | 2778978 | 7211 | TTTTCAGTTCRAGCTTTCTGA | 0.874 | 0.871 | 1.662 | 1.344 | 1.052 |
| 32 | 10 | 20586343 | 2681951 | 7212 | TTACAGTTAAYGGAGGAAACT | 0.582 | 0.561 | 1.051 | 1.429 | 1.098 |
| 32 | 10 | 20595556 | 16920183 | 7213 | AGTAAATAACYGGAGATTAGG | 0.226 | 0.231 | 0.566 | 1.219 | 0.993 |
| 32 | 10 | 20601545 | 2681942 | 7214 | AAAGCTATGTRTTCCCAAGTA | 0.906 | 0.861 | 0.242 | 0.512 | 0.928 |
| 32 | 10 | 20607483 | 2681947 | 7215 | AAATAAATTTRCGGCATTCTG | 0.086 | 0.087 | 0.129 | 0.325 | 1.074 |
| 32 | 10 | 20614437 | — | 7216 | TAAAATGGGAWCTAGAAACTG | 0.070 | 0.074 | 0.094 | 0.162 | 0.938 |
| 32 | 10 | 20620131 | 10828003 | 7217 | ATCGTCCTCTKCATGTCTTCA | 0.192 | 0.201 | 0.058 | 0.132 | 0.582 |
| 32 | 10 | 20627631 | 10508620 | 7218 | TGTCATCTACRCATCTATTTC | 0.049 | 0.046 | 0.089 | 0.193 | 0.312 |
| 32 | 10 | 20637777 | 10764218 | 7219 | ATTTCGTGAAYTGAAGAATGC | 0.766 | 0.763 | 0.181 | 0.090 | 0.495 |
| 32 | 10 | 20648041 | 12262812 | 7220 | GGCACCCAGTRATGTTCCTCC | 0.184 | 0.170 | 0.294 | 0.250 | 0.458 |
| 32 | 10 | 20673489 | 11011990 | 7221 | TTGGATCATTYTTTTCATGTT | 0.442 | 0.435 | 0.412 | 0.263 | 0.559 |
| 32 | 10 | 20679847 | 1412558 | 7222 | AATCGGGACARTTGTGAAAGT | 0.612 | 0.569 | 0.462 | 0.722 | 0.407 |
| 32 | 10 | 20686226 | 4748669 | 7223 | ATCCTGAGAGRGAGAAAAAAG | 0.374 | 0.364 | 0.434 | 0.929 | 0.588 |
| 32 | 10 | 20718364 | — | 7224 | GATTTTTTTTWTTCGACCAAA | 0.917 | 0.856 | 1.127 | 0.870 | 0.640 |
| 32 | 10 | 20724009 | 12777485 | 7225 | GAAAAACCCARCCATCTCATA | 0.139 | 0.137 | 1.035 | 0.852 | 0.638 |
| 32 | 10 | 20735677 | 12783104 | 7226 | TGGCACTGAAYACCGACGCCA | 1.773 | 2.151 | 1.195 | 1.036 | 1.062 |
| 32 | 10 | 20742566 | 17783778 | 7227 | AACTTCATACRTTCTGATATC | 0.426 | 0.429 | 0.804 | 0.921 | 1.046 |
| 32 | 10 | 20747068 | 16920492 | 7228 | TGAATACATCYCTGATATAAG | 0.735 | 0.730 | 1.219 | 0.860 | 1.024 |
| 32 | 10 | 20760926 | 11818220 | 7229 | AGATCTATCCYAATACACTGT | 0.170 | 0.171 | 0.489 | 1.023 | 1.119 |
| 32 | 10 | 20783154 | 10828054 | 7230 | CATAAATAAGKGAAAATTTTT | 0.874 | 0.885 | 0.387 | 1.311 | 1.177 |
| 32 | 10 | 20786206 | 12769394 | 7231 | TCCTCAATTCRTGATTAAAAC | 0.295 | 0.281 | 0.695 | 0.676 | 1.494 |
| 32 | 10 | 20812441 | 16920534 | 7232 | TGAATTAGAGRTGGTAGCTCA | 0.251 | 0.227 | 1.007 | 0.764 | 1.293 |
| 32 | 10 | 20821183 | 12262779 | 7233 | TGACAATTTGYATGAGAAAGA | 1.412 | 1.398 | 0.609 | 0.786 | 1.356 |
| 32 | 10 | 20830242 | 12782055 | 7234 | TTCTCCTTGGYCCCTGGAAAA | 0.773 | 0.751 | 0.809 | 1.256 | 0.761 |
| 32 | 10 | 20835803 | 1361360 | 7235 | AAGAGTAGTTYTGTTCACTTA | 0.066 | 0.101 | 1.161 | 1.059 | 0.873 |
| 32 | 10 | 20848770 | 7913924 | 7236 | AAATGAGGATWGTAATATGTA | 0.656 | 0.645 | 1.061 | 0.953 | 0.692 |
| 33 | 10 | 99966729 | 7078393 | 7237 | AGTTTGCAAGRTAATATGTTC | 0.133 | 0.130 | 0.250 | 0.572 | 0.947 |
| 33 | 10 | 99982196 | 11189521 | 7238 | GTATAGCTGAWACTTATATAG | 0.033 | 0.035 | 0.395 | 0.907 | 0.630 |
| 33 | 10 | 99989333 | 10748721 | 7239 | ATGCTAAGTCYTCCATGTTTT | 1.250 | 1.355 | 0.901 | 0.739 | 0.648 |
| 33 | 10 | 99995272 | 10786405 | 7240 | GTGCCCATGAYGATGATGAAT | 0.450 | 0.454 | 1.392 | 0.805 | 0.664 |
| 33 | 10 | 100006303 | 1983866 | 7241 | ATTTCTATTCWTCTTTCCTGC | 1.542 | 1.632 | 1.485 | 0.964 | 0.718 |
| 33 | 10 | 100013847 | 11189527 | 7242 | AGCTTGGATASCCTGTGATAG | 1.530 | 1.590 | 1.175 | 1.144 | 0.659 |
| 33 | 10 | 100021384 | 10786408 | 7243 | GGAACGTAGAYTGTAATAACA | 0.029 | 0.030 | 1.286 | 1.242 | 0.895 |
| 33 | 10 | 100026199 | 7915743 | 7244 | TCTGAATTGCYGTCACTGTGG | 0.296 | 0.299 | 0.806 | 1.002 | 0.973 |
| 33 | 10 | 100037491 | 10883059 | 7245 | CATGTTTGAGYGAATGGCCTG | 0.553 | 0.612 | 0.225 | 1.051 | 1.027 |
| 33 | 10 | 100047807 | 7903057 | 7246 | CTTTTTATTCYGCATGAGTCT | 0.569 | 0.569 | 0.346 | 0.870 | 1.067 |
| 33 | 10 | 100060547 | 17453254 | 7247 | CTTAAAAATCMCGGAGCTAGA | 0.277 | 0.289 | 0.408 | 0.379 | 0.752 |
| 33 | 10 | 100071747 | 2862297 | 7248 | ATGATAGTAAMCCAGGTGCAT | 0.341 | 0.350 | 0.631 | 0.390 | 0.770 |
| 33 | 10 | 100086156 | 4919215 | 7249 | TTCAACATTTYTGTCCCTGAA | 0.000 | 0.000 | 0.501 | 0.300 | 0.341 |
| 33 | 10 | 100092393 | 11189558 | 7250 | TCTGTGTGGCRATTGTTGAAA | 1.094 | 1.015 | 0.415 | 0.210 | 0.194 |
| 33 | 10 | 100105017 | 4919221 | 7251 | GAGATCCCATYAGCTCTCCTC | 0.321 | 0.329 | 0.269 | 0.166 | 0.328 |
| 33 | 10 | 100116376 | 17109506 | 7252 | TCACGTAGCCYTCACAACACA | 0.115 | 0.115 | 0.221 | 0.142 | 0.280 |
| 33 | 10 | 100125512 | 17109560 | 7253 | TTTCACTCATYATGTCTAGTG | 0.007 | 0.007 | 0.043 | 0.288 | 0.238 |
| 33 | 10 | 100132112 | 17109594 | 7254 | GGTGCCAGGTYAAAAAGTATA | 0.262 | 0.256 | 0.017 | 0.355 | 0.263 |
| 33 | 10 | 100138532 | 2274248 | 7255 | AGCTCCCAAAYGCAAATGTCT | 0.000 | 0.000 | 0.186 | 0.120 | 0.442 |
| 33 | 10 | 100149203 | 11812682 | 7256 | AGGGCCCTTAYGGAACTCAGT | 0.170 | 0.169 | 0.461 | 0.125 | 0.380 |
| 33 | 10 | 100157386 | 12763326 | 7257 | GGTAGGACGCRCGGGAGAACT | 0.879 | 0.834 | 0.378 | 0.239 | 0.343 |
| 33 | 10 | 100165072 | 2274245 | 7258 | GCTGGGCCGTYGGGAGTGTTG | 0.655 | 0.613 | 0.357 | 0.583 | 0.170 |
| 33 | 10 | 100172275 | 2296435 | 7259 | AGGTCTGAGTYAAGGTGCTTA | 0.035 | 0.035 | 0.543 | 0.525 | 0.122 |
| 33 | 10 | 100181485 | 11189602 | 7260 | GAGATGGGGASAGAGTTTGCA | 0.426 | 0.423 | 0.707 | 0.459 | 0.133 |
| 33 | 10 | 100190787 | 7071947 | 7261 | TGAGAACACGRGTGATGTTGA | 0.680 | 0.684 | 0.428 | 0.443 | 0.265 |
| 33 | 10 | 100195227 | 17109873 | 7262 | TTGAGAAAGGRCAAGCTGGAG | 1.183 | 1.122 | 0.526 | 0.199 | 0.367 |
| 33 | 10 | 100205957 | 7093411 | 7263 | CATGACCACCRGAGGCTAAGT | 0.011 | 0.010 | 0.399 | 0.120 | 0.328 |
| 33 | 10 | 100216639 | 17109945 | 7264 | CTGATCTTAGRACATTGTCTG | 0.251 | 0.258 | 0.159 | 0.287 | 0.387 |
| 33 | 10 | 100230295 | 17458353 | 7265 | AGCTCCAAACRGCAGGGGAAT | 0.196 | 0.162 | 0.009 | 0.399 | 0.414 |
| 33 | 10 | 100241958 | 2017304 | 7266 | ACATTTGGAARAAAGAGATAT | 0.002 | 0.002 | 0.121 | 0.294 | 0.392 |
| 33 | 10 | 100256082 | 1932796 | 7267 | TTTTATTTTTRGGAGTTTGCT | 0.191 | 0.181 | 0.286 | 0.135 | 0.549 |
| 33 | 10 | 100266875 | 17537531 | 7268 | AGATCCTAAGRCCCAGAGCAG | 0.847 | 0.823 | 0.356 | 0.362 | 0.567 |
| 33 | 10 | 100275239 | 12412681 | 7269 | GCTGGGAAGAYGGGCCTAAGG | 0.776 | 0.763 | 0.500 | 0.433 | 0.419 |
| 33 | 10 | 100282154 | 7096013 | 7270 | TTCTGCCTCTSCTTCGTGATC | 0.289 | 0.278 | 0.868 | 0.575 | 0.265 |
| 33 | 10 | 100283172 | 665142 | 7271 | TGTTTCGTAAYGTTAATCACA | 0.410 | 0.430 | 0.698 | 0.754 | 0.353 |
| 33 | 10 | 100297705 | 11189671 | 7272 | GCAAGAGGTCWCTTCTTCTAC | 1.007 | 0.989 | 0.669 | 0.718 | 0.459 |
| 33 | 10 | 100305542 | 10509724 | 7273 | TTTGACAACAYGAAAATAACG | 0.519 | 0.505 | 0.791 | 0.569 | 0.443 |
| 33 | 10 | 100316583 | 544176 | 7274 | TAGTTCCTGTYACTCCAGATG | 0.649 | 0.696 | 0.599 | 0.478 | 1.423 |
| 33 | 10 | 100332431 | 11189687 | 7275 | CTTTTATGGCRTAACTGTCTC | 0.506 | 0.489 | 0.338 | 0.639 | 1.655 |
| 33 | 10 | 100340328 | 10883146 | 7276 | AGCCATGATGYGCTTATCTGT | 0.022 | 0.022 | 0.332 | 0.531 | 1.715 |
| 33 | 10 | 100351260 | 560749 | 7277 | ACTACCAACCSTGATTATCTC | 0.395 | 0.404 | 0.362 | 1.517 | 1.732 |
| 33 | 10 | 100359992 | 526465 | 7278 | TCCACACCATRGCTTGGCAGT | 0.506 | 0.505 | 0.216 | 1.803 | 1.916 |
| 33 | 10 | 100370950 | 10883151 | 7279 | ACTAGTCCAGKTATATTGTAA | 0.735 | 0.773 | 2.104 | 2.036 | 1.730 |
| 33 | 10 | 100379797 | 11189719 | 7280 | GCTAACTGCTWTATTTAACTT | 0.101 | 0.103 | 2.161 | 2.037 | 1.461 |
| 33 | 10 | 100389254 | 17110455 | 7281 | CATCTCTCAGKGGCTCTACTG | 3.465 | 3.488 | 2.680 | 2.241 | 1.644 |
| 33 | 10 | 100396734 | 563060 | 7282 | TAAACCTGTAYTCCAGATCCT | 0.926 | 0.932 | 2.753 | 1.974 | 1.465 |
| 33 | 10 | 100402147 | 528556 | 7283 | TTCAACAACCRGTCCCACATA | 1.021 | 1.037 | 2.855 | 1.733 | 1.608 |
| 33 | 10 | 100409703 | 475945 | 7284 | ACTTCACTGARTACTTGTGAG | 0.980 | 0.973 | 0.884 | 1.823 | 1.781 |
| 33 | 10 | 100424345 | 3021452 | 7285 | TCTCAAGATTRTGTATAAAAG | 0.464 | 0.451 | 0.632 | 1.708 | 1.772 |
| 33 | 10 | 100425944 | 965934 | 7286 | ATGAGTGGCCRGTGTTATTAA | 0.017 | 0.017 | 0.702 | 0.946 | 1.829 |
| 33 | 10 | 100445675 | 10748750 | 7287 | ACCAGCTCTTRAATGAATAGA | 0.394 | 0.395 | 0.362 | 0.893 | 2.177 |
| 33 | 10 | 100455887 | 17110699 | 7288 | CTCAAATTCAYGTAGTTAGTG | 1.229 | 1.221 | 0.524 | 0.708 | 2.207 |
| 33 | 10 | 100463182 | 17110721 | 7289 | ATCCTCCAAAYTGTAATTCTT | 0.011 | 0.011 | 0.880 | 0.582 | 1.212 |
| 33 | 10 | 100471417 | 17110744 | 7290 | TCTAGGAGGGYGATGACATTC | 0.942 | 0.945 | 0.873 | 1.133 | 0.991 |
| 33 | 10 | 100483316 | 17110762 | 7291 | TGGTACTTACKGCTGCCTTTA | 0.712 | 0.657 | 0.521 | 1.330 | 0.843 |
| 33 | 10 | 100491881 | 11189793 | 7292 | GGAATACGAARAACTTCAGGC | 0.408 | 0.396 | 1.559 | 1.225 | 0.715 |
| 33 | 10 | 100500931 | 10786467 | 7293 | TTGACAGAGASGATTAACATT | 0.549 | 0.542 | 1.289 | 0.816 | 0.962 |
| 33 | 10 | 100524479 | 17110835 | 7294 | TAAGTAACCGRTACTGGCAGC | 2.011 | 1.902 | 0.991 | 0.925 | 1.202 |
| 33 | 10 | 100530404 | 17110853 | 7295 | AGGCTTTTGAYTGCTTGGCTA | 0.494 | 0.488 | 0.900 | 0.746 | 1.189 |
| 33 | 10 | 100541536 | 1414971 | 7296 | TGCCTGACATWCCACAGCTAG | 0.203 | 0.203 | 0.797 | 0.997 | 0.853 |
| 33 | 10 | 100542446 | 10883202 | 7297 | GGGGTCAGAGYGGTGGTTGTT | 0.109 | 0.110 | 0.162 | 1.194 | 0.876 |
| 33 | 10 | 100566461 | 10883210 | 7298 | AACTGCCTCCRTAAACAACTT | 0.383 | 0.376 | 0.480 | 1.058 | 0.761 |
| 33 | 10 | 100574864 | 10786475 | 7299 | TGTAATACTTRTAACAAAAGA | 0.389 | 0.393 | 0.703 | 0.394 | 0.736 |
| 33 | 10 | 100581061 | 17110919 | 7300 | AGTGTGCTTCYTCCAAGGCAA | 1.391 | 1.409 | 0.857 | 0.325 | 0.742 |
| 33 | 10 | 100587657 | 7084345 | 7301 | ATATCTGTGCRGTTTTGCTTA | 0.648 | 0.669 | 0.677 | 0.422 | 0.779 |
| 33 | 10 | 100593685 | 2862447 | 7302 | TATTGCTTCARTTCTAATTCT | 0.407 | 0.400 | 0.580 | 0.555 | 0.393 |
| 33 | 10 | 100607163 | 7091295 | 7303 | ATTGTGAGTTSGAATAGAAAC | 0.045 | 0.047 | 0.307 | 0.552 | 0.354 |
| 33 | 10 | 100610499 | 10883221 | 7304 | TAAGGCCTAAYTAAAATGCCA | 0.169 | 0.171 | 0.281 | 0.644 | 0.451 |
| 33 | 10 | 100631865 | 10786481 | 7305 | CCTTTCTCCAYTAGATAAGCA | 0.550 | 0.549 | 0.231 | 0.335 | 0.537 |
| 33 | 10 | 100638398 | 4919262 | 7306 | CTCAATTACCRTCTCCTTTCT | 0.606 | 0.599 | 0.438 | 0.238 | 0.510 |
| 33 | 10 | 100650291 | 978851 | 7307 | TAGTTATGCAYGAACATTATA | 0.290 | 0.337 | 0.494 | 0.292 | 0.506 |
| 33 | 10 | 100656085 | — | 7308 | CATGGAGTTTSATTGTGAAGA | 0.404 | 0.680 | 0.348 | 0.407 | 0.430 |
| 33 | 10 | 100662056 | 17110998 | 7309 | CTTCCAGCAAYGTCATTTGGT | 0.324 | 0.293 | 0.391 | 0.424 | 0.407 |
| 33 | 10 | 100684372 | 11492738 | 7310 | TCTGGCTTGCRGAGTTTCTGC | 0.273 | 0.275 | 0.475 | 0.360 | 0.470 |
| 33 | 10 | 100695326 | 17111002 | 7311 | CAGAAAATACYTGCTATATAA | 0.654 | 0.618 | 0.278 | 0.507 | 0.784 |
| 33 | 10 | 100702951 | 4444007 | 7312 | TAAGATGTTCMATAAAAGGCT | 0.482 | 0.472 | 0.303 | 0.583 | 0.787 |
| 33 | 10 | 100709362 | 11189907 | 7313 | TAAACTCCAGYAAAATCTCCC | 0.256 | 0.250 | 0.627 | 0.594 | 0.680 |
| 33 | 10 | 100724399 | 2902256 | 7314 | CGTTCCTACAWAAAGATCGTG | 0.358 | 0.364 | 0.568 | 0.942 | 0.654 |
| 33 | 10 | 100729760 | 17111026 | 7315 | TCAGATGAACRCTGCAGGCCA | 1.026 | 1.008 | 0.694 | 0.895 | 0.638 |
| 33 | 10 | 100749687 | 10509727 | 7316 | TGCAGTATGAYAGATGAATTA | 0.546 | 0.543 | 1.258 | 0.698 | 0.721 |
| 33 | 10 | 100759845 | 12249519 | 7317 | CCAATAAACCRATAATCTAAA | 0.735 | 0.726 | 1.126 | 0.727 | 1.418 |
| 33 | 10 | 100768211 | 10509728 | 7318 | TGTTGGACAAMCTTTGGCTAA | 1.323 | 1.295 | 0.666 | 0.723 | 1.468 |
| 33 | 10 | 100789378 | 2487890 | 7319 | TTTGCAGATASAGAGTAAGTC | 0.178 | 0.182 | 0.665 | 1.022 | 1.420 |
| 33 | 10 | 100797780 | 2862507 | 7320 | GCAATTAATAWGCTGTGGACT | 0.130 | 0.128 | 0.428 | 1.820 | 1.405 |
| 33 | 10 | 100814705 | 11189954 | 7321 | CCTCCTCTACYATTATTCTCA | 0.000 | 0.000 | 0.269 | 1.689 | 1.793 |
| 33 | 10 | 100819181 | 1342576 | 7322 | AATTACTGTAYAGATTTTGGA | 0.239 | 0.234 | 1.751 | 1.641 | 1.716 |
| 33 | 10 | 100831915 | 4462267 | 7323 | AGGGTAGGGGYCTGCTACATG | 1.001 | 0.992 | 1.758 | 1.142 | 1.539 |
| 33 | 10 | 100841378 | 10883258 | 7324 | AAATATGCTCSATTTGGCATT | 2.627 | 2.974 | 1.758 | 1.614 | 1.373 |
| 33 | 10 | 100848616 | 10509729 | 7325 | TAAAGTGCTASGGTTCTCATA | 0.602 | 0.614 | 1.676 | 1.412 | 1.131 |
| 33 | 10 | 100859904 | 7089572 | 7326 | TAAAAGAAAARGACATGCTCT | 0.000 | 0.000 | 1.922 | 1.415 | 0.877 |
| 33 | 10 | 100871811 | 1857184 | 7327 | ATTATGTATAYGTATGTGCTT | 0.261 | 0.320 | 0.719 | 1.365 | 1.879 |
| 33 | 10 | 100890445 | 11189991 | 7328 | GACAACTGAAYTCGAACCCAT | 1.539 | 1.525 | 0.886 | 1.087 | 1.712 |
| 33 | 10 | 100895445 | 1935665 | 7329 | TTGACACCAAYGATTTTGGAA | 0.070 | 0.068 | 0.717 | 0.501 | 1.611 |
| 33 | 10 | 100939639 | 3905193 | 7330 | TACCAAGTTGWTGCTGTTCTT | 0.998 | 1.011 | 0.609 | 1.691 | 1.589 |
| 33 | 10 | 100968381 | 10786524 | 7331 | AAAACCCCAARTGAAGACATT | 0.171 | 0.171 | 0.158 | 1.431 | 1.486 |
| 33 | 10 | 100973931 | 10748766 | 7332 | CACTGTCTTTYACCATATCAA | 0.008 | 0.008 | 2.037 | 1.287 | 0.892 |
| 33 | 10 | 100979909 | 12573571 | 7333 | GTCTGGAAGGMCTGACTTTAA | 0.176 | 0.177 | 1.619 | 0.930 | 0.902 |
| 33 | 10 | 100989074 | 17094130 | 7334 | GTAAGAAAACRTGAAGATTAA | 4.480 | 4.422 | 1.642 | 1.105 | 0.823 |
| 33 | 10 | 100996728 | 10786525 | 7335 | TTCTCAAAAAMAAAAATAATC | 0.114 | 0.108 | 1.757 | 0.780 | 0.828 |
| 33 | 10 | 101031490 | 12570965 | 7336 | AAGATTGTTARGCAAGGAACA | 0.044 | 0.043 | 2.086 | 0.893 | 0.729 |
| 33 | 10 | 101041410 | 17111428 | 7337 | ATCCTATCTCRTTTTTTCTTT | 0.077 | 0.069 | 0.010 | 0.998 | 0.779 |
| 33 | 10 | 101060139 | — | 7338 | ATTGATTAGTKTTTCCTGTGT | 0.334 | 0.330 | 0.035 | 1.107 | 0.665 |
| 33 | 10 | 101071375 | 10509733 | 7339 | GAACCATTGCYCTATAAAGAA | 0.058 | 0.056 | 0.056 | 0.099 | 0.652 |
| 33 | 10 | 101083674 | 11190059 | 7340 | GTATTTAGGTRGAGCACTAAA | 0.440 | 0.456 | 0.100 | 0.102 | 0.865 |
| 33 | 10 | 101090322 | 4919300 | 7341 | TAGTACTGTCWTAAAACGAGT | 0.187 | 0.186 | 0.324 | 0.152 | 0.946 |
| 33 | 10 | 101096097 | 12570708 | 7342 | ACTTCATTACRTGTGAACACT | 0.337 | 0.333 | 0.382 | 0.158 | 0.212 |
| 33 | 10 | 101108239 | 10509734 | 7343 | TCGTTTCAGAMTTTGGTCCTT | 1.034 | 1.033 | 0.360 | 0.245 | 0.220 |
| 33 | 10 | 101115591 | 1892509 | 7344 | AATTATCAGARGGTCCCAGAG | 0.208 | 0.207 | 0.320 | 0.374 | 0.502 |
| 33 | 10 | 101123989 | 4919305 | 7345 | GTTCTCTACAKTACCATCACT | 0.393 | 0.388 | 0.495 | 0.558 | 0.792 |
| 33 | 10 | 101134457 | 17111633 | 7346 | TGCTTAAAAARAGGGTTAGAG | 0.086 | 0.088 | 0.290 | 0.537 | 0.775 |
| 33 | 10 | 101140337 | 3793932 | 7347 | GTTTTGGGATMTGTCCCATTC | 0.768 | 0.767 | 0.645 | 0.909 | 0.985 |
| 33 | 10 | 101147807 | 3750897 | 7348 | TTAGTTCTTCSCTAACCTGGA | 0.497 | 0.500 | 0.522 | 1.015 | 0.870 |
| 33 | 10 | 101160190 | 2862611 | 7349 | CTCAACTTTTMACTCCACCCT | 1.079 | 1.043 | 1.099 | 1.055 | 1.036 |
| 33 | 10 | 101168146 | 11594790 | 7350 | AAACTCCCCARCATGGAATCT | 0.129 | 0.130 | 1.477 | 1.129 | 1.111 |
| 33 | 10 | 101178468 | 4919310 | 7351 | GTATACATCCSGCTGCAAAGA | 1.621 | 1.669 | 1.433 | 1.106 | 0.973 |
| 33 | 10 | 101188327 | 2494654 | 7352 | AGTCACATCTRTTATATAGTC | 1.355 | 1.325 | 1.361 | 1.200 | 1.001 |
| 33 | 10 | 101203270 | 2494657 | 7353 | ATCCTGGTTAYGAATAGTCAG | 0.157 | 0.155 | 1.367 | 1.404 | 0.907 |
| 33 | 10 | 101210464 | 17111730 | 7354 | AGGCCCTGACRTAATTTCAAC | 0.741 | 0.759 | 0.927 | 1.182 | 0.968 |
| 33 | 10 | 101217569 | 10883350 | 7355 | AGATAAACCCYTGGGCAGCAA | 0.192 | 0.194 | 0.658 | 1.253 | 0.854 |
| 33 | 10 | 101219517 | 2862596 | 7356 | ACTTGAAAATYTCTATACGTC | 0.952 | 0.978 | 0.753 | 0.664 | 0.835 |
| 34 | 11 | 11697651 | 1471014 | 7357 | AATACAATGCYAAGTTTTAGA | 0.887 | 0.848 | 0.157 | 0.371 | 0.281 |
| 34 | 11 | 11704216 | 2233743 | 7358 | GTCTCCTAACYACTCAGTCTC | 0.180 | 0.172 | 0.310 | 0.128 | 0.289 |
| 34 | 11 | 11710726 | 1455252 | 7359 | ATGAATCACARTATACCAATA | 0.410 | 0.388 | 0.484 | 0.124 | 0.710 |
| 34 | 11 | 11717646 | 17446021 | 7360 | AGGTGGGAGGYGTGAAATCAG | 0.582 | 0.542 | 0.188 | 0.111 | 0.628 |
| 34 | 11 | 11723285 | 11022014 | 7361 | ACCAGTGGGAYGTAGCTAAAA | 0.482 | 0.492 | 0.160 | 0.198 | 0.488 |
| 34 | 11 | 11730217 | 897362 | 7362 | ACAAGATAAAYGGATTTGGAA | 0.014 | 0.014 | 0.092 | 0.775 | 0.568 |
| 34 | 11 | 11737183 | 16924665 | 7363 | AAAAAGCATAYGTAATCCTCT | 0.177 | 0.172 | 0.082 | 0.798 | 1.089 |
| 34 | 11 | 11746757 | 16910054 | 7364 | AATCAGTTTTRTGGAGTGCAT | 0.101 | 0.093 | 1.028 | 0.885 | 1.111 |
| 34 | 11 | 11756544 | 1455264 | 7365 | TCTTCATAAAYGTTTCTGACT | 0.518 | 0.493 | 1.110 | 0.877 | 1.113 |
| 34 | 11 | 11763136 | 16910116 | 7366 | AAGCTTGTCTRCATGGGGGCT | 2.970 | 2.740 | 1.363 | 1.474 | 1.812 |
| 34 | 11 | 11770132 | 7940987 | 7367 | ATTGTTTTCTWCAATCATAGC | 0.122 | 0.122 | 1.701 | 1.470 | 1.762 |
| 34 | 11 | 11781525 | 2054115 | 7368 | TTAACACCTCRAGAGATACAG | 0.676 | 0.642 | 2.480 | 1.918 | 2.190 |
| 34 | 11 | 11787807 | 7120182 | 7369 | ATATATGGAAWGAAAGCCTTT | 0.459 | 0.467 | 0.888 | 2.936 | 2.151 |
| 34 | 11 | 11793828 | 12575179 | 7370 | TCTGTGATTAMCATGACTTAA | 1.882 | 1.898 | 1.447 | 2.886 | 2.321 |
| 34 | 11 | 11801092 | 1542379 | 7371 | TAGATGTTAARTTCATACTTG | 0.132 | 0.136 | 2.043 | 2.055 | 2.936 |
| 34 | 11 | 11807368 | 7944875 | 7372 | GATGATAATTRGGTTTAAAAG | 1.003 | 0.975 | 1.889 | 2.195 | 2.740 |
| 34 | 11 | 11821720 | 7926410 | 7373 | TGTGTATTTCRTTTTAGAGCT | 2.262 | 2.012 | 1.645 | 1.987 | 2.787 |
| 34 | 11 | 11824326 | 17378517 | 7374 | TCTCTTTTAAYAGCATCCCAA | 0.314 | 0.314 | 1.863 | 2.412 | 2.772 |
| 34 | 11 | 11850597 | 7935751 | 7375 | GGAAGCAAGTYAAAGAATTTG | 1.995 | 1.952 | 1.754 | 1.724 | 3.062 |
| 34 | 11 | 11873223 | 7111921 | 7376 | ATTAATATTGYTGATATTATT | 0.000 | 0.000 | 2.182 | 1.870 | 2.767 |
| 34 | 11 | 11879369 | 4243945 | 7377 | CAAAAGAAAARAAAAAGTCTT | 0.002 | 0.002 | 1.976 | 2.905 | 2.481 |
| 34 | 11 | 11887874 | 4910429 | 7378 | TAATTTCATAYAGTGCCTTTG | 2.977 | 3.180 | 1.258 | 3.233 | 2.264 |
| 34 | 11 | 11911794 | 6485303 | 7379 | TGAGACATAAYTGGTGTCAAT | 0.051 | 0.052 | 3.108 | 2.932 | 2.320 |
| 34 | 11 | 11930555 | 16910238 | 7380 | ATAGGAGCCARTAGAGAAACA | 0.453 | 0.464 | 3.687 | 2.533 | 2.556 |
| 34 | 11 | 11936250 | 1979687 | 7381 | ACTTGCTATCRCACAAAAGAG | 3.982 | 3.375 | 2.819 | 2.533 | 2.456 |
| 34 | 11 | 11944883 | 7480000 | 7382 | AGCATCAACASGGGAAAAAAA | 2.609 | 2.458 | 2.689 | 2.547 | 2.418 |
| 34 | 11 | 11951408 | 1552796 | 7383 | GGACTGGAGCYGACAGACACT | 0.708 | 0.708 | 2.653 | 2.178 | 2.153 |
| 34 | 11 | 11957612 | 4757519 | 7384 | CCCAGGAGATRAGTGCCAGAG | 0.486 | 0.478 | 1.354 | 2.378 | 2.049 |
| 34 | 11 | 11964354 | 7113678 | 7385 | ATTATGAGACYATGGAAAATT | 0.000 | 0.000 | 0.478 | 2.270 | 2.125 |
| 34 | 11 | 11971244 | 987663 | 7386 | TCTAGCATTTRCACTGAAAGT | 0.087 | 0.090 | 0.209 | 0.798 | 1.523 |
| 34 | 11 | 11977280 | 17380453 | 7387 | TTACAAGTCCRGGTGTCTAGT | 0.688 | 0.680 | 0.118 | 0.169 | 1.662 |
| 34 | 11 | 11983640 | 6485369 | 7388 | TGGTAGCATAYAGAATAGTAA | 0.108 | 0.105 | 0.058 | 0.112 | 1.631 |
| 34 | 11 | 11998072 | 4500466 | 7389 | TTCCTCTATCRTTGTCATGCT | 0.147 | 0.143 | 0.138 | 0.116 | 0.749 |
| 34 | 11 | 12008425 | 4757082 | 7390 | CCCATGGAAGRGCAAAAGGCA | 0.058 | 0.054 | 0.083 | 0.196 | 0.227 |
| 34 | 11 | 12020875 | 16910400 | 7391 | TTAGTAAATAYGCTGACTTCC | 0.405 | 0.375 | 0.151 | 0.189 | 0.138 |
| 34 | 11 | 12029564 | 1994328 | 7392 | CTCTCTGAGGRTGAAATGAGA | 0.442 | 0.444 | 0.329 | 0.212 | 0.089 |
| 34 | 11 | 12040379 | 11022142 | 7393 | GAATCTCTAAYGGACTGAGGA | 0.527 | 0.480 | 0.348 | 0.220 | 0.068 |
| 34 | 11 | 12050034 | 2018347 | 7394 | TACCATGTGCRGTTACTAATG | 0.742 | 0.757 | 0.536 | 0.210 | 0.150 |
| 34 | 11 | 12056845 | 10831721 | 7395 | TTTCTCCCCTYATTGGCCTCG | 0.074 | 0.072 | 0.432 | 0.200 | 0.081 |
| 34 | 11 | 12064047 | 9665967 | 7396 | CTTAAGAAGARCCTAGAGCAA | 0.850 | 0.819 | 0.304 | 0.161 | 0.103 |
| 34 | 11 | 12070720 | 11022169 | 7397 | TCAGAGCTCCRTGCAGGCCCA | 0.179 | 0.185 | 0.080 | 0.238 | 0.123 |
| 34 | 11 | 12078670 | 7111481 | 7398 | CTAGCGAGGAYGCGAAACTTC | 0.083 | 0.087 | 0.094 | 0.166 | 0.177 |
| 34 | 11 | 12094784 | 7947696 | 7399 | TTCAGAGCCCRAGGGAGTGAT | 0.018 | 0.018 | 0.091 | 0.089 | 0.336 |
| 34 | 11 | 12107131 | 7940840 | 7400 | TGCCTTCTTARTCTCTGAGGT | 0.173 | 0.177 | 0.075 | 0.136 | 0.336 |
| 34 | 11 | 12112990 | 16910593 | 7401 | TGGAAGGTTTYGAGGAGGCTC | 0.843 | 0.828 | 0.137 | 0.073 | 0.513 |
| 34 | 11 | 12119025 | 1009087 | 7402 | AGACTGAGGARGAAGGACAGG | 0.081 | 0.081 | 0.245 | 0.290 | 0.519 |
| 34 | 11 | 12126901 | 10831742 | 7403 | GTGCAAAACCRTCATACAAAC | 0.328 | 0.334 | 0.351 | 0.399 | 0.669 |
| 34 | 11 | 12132301 | 4756783 | 7404 | TGGGGGACTTRGCCCTGACCT | 0.398 | 0.389 | 0.516 | 0.808 | 0.495 |
| 34 | 11 | 12139612 | 7131034 | 7405 | AAGTTTTGAGMAGAGACTGTT | 0.462 | 0.470 | 0.688 | 0.970 | 0.555 |
| 34 | 11 | 12149786 | 10831746 | 7406 | CTGCAGGTAAYTGAGATGCTG | 1.288 | 1.234 | 1.163 | 0.920 | 0.702 |
| 34 | 11 | 12159487 | 7926135 | 7407 | AGAAATTCTCRATTAAAGTCC | 0.473 | 0.464 | 1.340 | 0.981 | 0.836 |
| 34 | 11 | 12172973 | 10437601 | 7408 | GAGGCTATAARAAGAAAATAT | 1.281 | 1.328 | 1.362 | 1.018 | 0.906 |
| 34 | 11 | 12179611 | 10831761 | 7409 | GTGGGTGCGCRTTCCCTTGCC | 0.797 | 0.801 | 0.878 | 1.093 | 1.235 |
| 34 | 11 | 12186231 | 2279390 | 7410 | TCGTGAAGGTKCTAGAGCCTC | 0.612 | 0.650 | 0.874 | 1.134 | 1.306 |
| 34 | 11 | 12191983 | 11022250 | 7411 | TCCCGGGTGAKTTTGTGCAAT | 0.115 | 0.117 | 0.588 | 0.880 | 1.380 |
| 34 | 11 | 12198606 | 4237704 | 7412 | CGGAGCCCCCRTGTTTCTCTG | 0.433 | 0.431 | 0.478 | 1.475 | 1.293 |
| 34 | 11 | 12212122 | 2010576 | 7413 | ATTTGGGTATRTGTGTTGCTA | 0.742 | 0.682 | 0.404 | 0.974 | 1.468 |
| 34 | 11 | 12218140 | 2706631 | 7414 | ATACAGTTTGMAGGAGCTTTG | 0.554 | 0.567 | 1.390 | 0.895 | 1.430 |
| 34 | 11 | 12224116 | 11608031 | 7415 | TCCAGCTGACKGGGCCCTTCA | 0.000 | 0.000 | 1.247 | 0.657 | 1.222 |
| 34 | 11 | 12231441 | 1826608 | 7416 | GTCTCCAAGCYGACCTTGCCT | 1.847 | 1.770 | 1.183 | 1.069 | 0.902 |
| 35 | 11 | 19443295 | 12792754 | 7417 | ACTTTCCACTYTCCTTAGGTA | 0.250 | 0.245 | 0.293 | 0.520 | 0.203 |
| 35 | 11 | 19452545 | 2632034 | 7418 | GATGTGTACAYTATGATACAA | 0.207 | 0.201 | 0.533 | 0.520 | 0.323 |
| 35 | 11 | 19464393 | 10833124 | 7419 | ATAGTCTCTTYCTGGGACCCT | 0.337 | 0.328 | 0.708 | 0.473 | 0.246 |
| 35 | 11 | 19467603 | 11025154 | 7420 | ACAGGCAGATYTGAGTAACTT | 1.491 | 1.473 | 0.658 | 0.308 | 0.236 |
| 35 | 11 | 19475674 | 2403494 | 7421 | CCCAGGCTCTYTTCCTATTTT | 0.862 | 0.866 | 0.604 | 0.419 | 0.350 |
| 35 | 11 | 19486762 | 16936838 | 7422 | CTAAGTTCCCYGTCCTGTACT | 0.037 | 0.036 | 0.513 | 0.351 | 0.380 |
| 35 | 11 | 19496685 | 2702735 | 7423 | TTAGAACAAARTACCCTGCCT | 0.030 | 0.030 | 0.233 | 0.349 | 0.338 |
| 35 | 11 | 19504649 | 7127676 | 7424 | TTTTTCACCCRTTGTTTACTC | 0.127 | 0.128 | 0.033 | 0.465 | 0.290 |
| 35 | 11 | 19518021 | 16936939 | 7425 | AGATAGGTCGYGGAGGGGGAC | 0.759 | 0.827 | 0.065 | 0.172 | 0.249 |
| 35 | 11 | 19523724 | 11025179 | 7426 | TAGTGCATTTYATTTCCTAGA | 0.002 | 0.002 | 0.257 | 0.129 | 0.279 |
| 35 | 11 | 19531121 | 11025181 | 7427 | TCTGCAATTGYGGAGTGAAAA | 0.236 | 0.234 | 0.382 | 0.141 | 0.361 |
| 35 | 11 | 19543750 | 4757007 | 7428 | TTGATTTTCCWATGGTGATAA | 0.726 | 0.742 | 0.315 | 0.149 | 0.217 |
| 35 | 11 | 19550426 | 1487199 | 7429 | TAAAGCCCTCMGTCCCTTCTT | 0.000 | 0.000 | 0.356 | 0.215 | 0.137 |
| 35 | 11 | 19556547 | 10741780 | 7430 | CCTGATGTCAYTGAGCCTCTA | 0.599 | 0.612 | 0.273 | 0.199 | 0.889 |
| 35 | 11 | 19569906 | 4757821 | 7431 | TTTATCTTGARGTCCTGAAAT | 0.114 | 0.116 | 0.154 | 0.379 | 1.099 |
| 35 | 11 | 19576520 | 1471903 | 7432 | TAGCTTCAGGKAATTCTTTTT | 0.029 | 0.030 | 0.252 | 0.415 | 1.024 |
| 35 | 11 | 19584103 | 10833134 | 7433 | CATACCGTTTYAGACGGAGGC | 0.387 | 0.391 | 0.300 | 1.356 | 1.118 |
| 35 | 11 | 19592029 | 17599506 | 7434 | TAAAACCTAARTTGCCTCATC | 0.646 | 0.652 | 0.392 | 1.434 | 1.991 |
| 35 | 11 | 19607973 | 7116289 | 7435 | TGGAATGGCCRCATCAACCCT | 0.773 | 0.753 | 1.995 | 1.196 | 2.735 |
| 35 | 11 | 19614433 | 16937060 | 7436 | TACAAAGCGGMTCTTGGTCTG | 0.355 | 0.365 | 2.448 | 1.618 | 3.108 |
| 35 | 11 | 19630248 | 1559667 | 7437 | CCAATTAAATRAAGCCTATTA | 3.341 | 2.951 | 1.808 | 2.759 | 3.108 |
| 35 | 11 | 19638525 | 16937087 | 7438 | CTGGACATTAKGGTTCATACG | 0.625 | 0.626 | 2.029 | 3.375 | 3.488 |
| 35 | 11 | 19647977 | 10833153 | 7439 | TCCTTCCTCCYTTGTCCTGGG | 0.075 | 0.074 | 3.105 | 3.474 | 3.665 |
| 35 | 11 | 19653940 | 2216997 | 7440 | TACAGCAATTKGTAATTCACG | 1.141 | 1.204 | 3.271 | 3.604 | 3.687 |
| 35 | 11 | 19659944 | 7113050 | 7441 | AGACCTGTGGRCACATCTGAA | 2.724 | 2.885 | 3.322 | 4.036 | 3.710 |
| 35 | 11 | 19663584 | 752459 | 7442 | AGGTATGGCCRGGCTGAGGAG | 3.029 | 3.156 | 3.676 | 3.633 | 3.488 |
| 35 | 11 | 19687211 | 890136 | 7443 | CTTATAGGCCRTTTTTATAAT | 2.155 | 2.231 | 3.748 | 3.467 | 3.386 |
| 35 | 11 | 19696994 | — | 7444 | CACAAATATAYACACATGGAA | 0.000 | 0.000 | 3.091 | 3.551 | 3.559 |
| 35 | 11 | 19707031 | 1559665 | 7445 | CAAGGAACGTRTGCAGAGAAA | 0.631 | 0.663 | 1.379 | 2.883 | 2.492 |
| 35 | 11 | 19724079 | 12223569 | 7446 | GATCAGGGCTKACCGTATTTA | 0.426 | 0.396 | 0.547 | 2.000 | 2.291 |
| 35 | 11 | 19731568 | 16937173 | 7447 | CATATTTGTGKTAGTAAGTCA | 0.190 | 0.189 | 0.384 | 0.981 | 2.440 |
| 35 | 11 | 19738767 | 7118473 | 7448 | GCAAACGGCAYCTTTTCAAAA | 0.889 | 0.862 | 0.419 | 0.289 | 2.540 |
| 35 | 11 | 19744864 | 12797612 | 7449 | CCACTTGGGAWTGGCAACTAG | 0.095 | 0.097 | 0.338 | 0.205 | 1.622 |
| 35 | 11 | 19751009 | 1425237 | 7450 | AACTGAAATAYGTTATCTGGG | 0.687 | 0.677 | 0.319 | 0.207 | 0.769 |
| 35 | 11 | 19764639 | 10766594 | 7451 | AGGTCATTGAKATTGCATACA | 0.177 | 0.181 | 0.073 | 0.380 | 0.384 |
| 35 | 11 | 19769834 | 10833171 | 7452 | ATCCAGAGAGYGGGCACTCAG | 0.126 | 0.127 | 0.202 | 0.362 | 0.408 |
| 35 | 11 | 19781820 | 10833174 | 7453 | GATTCCCCCARTGGTAGAGGA | 0.107 | 0.103 | 0.369 | 0.261 | 0.399 |
| 35 | 11 | 19790982 | 10833178 | 7454 | TAAGAGGAGASCAGAATTCAG | 0.602 | 0.609 | 0.337 | 0.393 | 0.327 |
| 35 | 11 | 19795239 | 1425235 | 7455 | ATAATCACCAYGAGTGCACAC | 1.180 | 1.113 | 0.472 | 0.360 | 0.368 |
| 35 | 11 | 19812716 | 1816814 | 7456 | ATGGAAATACMACTTGCATCT | 0.144 | 0.142 | 0.713 | 0.474 | 0.210 |
| 35 | 11 | 19820738 | 16937196 | 7457 | ACGATGCCCCWTCACTCCAAG | 0.479 | 0.477 | 0.680 | 0.455 | 0.274 |
| 35 | 11 | 19827846 | 12287043 | 7458 | ACATGTTGCARAACCAAGAAG | 0.642 | 0.618 | 0.452 | 0.553 | 0.195 |
| 35 | 11 | 19842939 | 2014079 | 7459 | CTGGGGAGGTRCATCCTGGTC | 0.589 | 0.567 | 0.405 | 0.374 | 0.189 |
| 35 | 11 | 19848438 | 1838053 | 7460 | ACATGATTTCSTCCTGGTAGA | 0.604 | 0.612 | 0.380 | 0.208 | 0.172 |
| 35 | 11 | 19854870 | 12803334 | 7461 | ACAGTGGATGWCCCCTGATCC | 0.064 | 0.065 | 0.176 | 0.216 | 0.189 |
| 35 | 11 | 19861136 | 2625301 | 7462 | TTACATCAGTSGCTCGGTCTC | 0.380 | 0.393 | 0.154 | 0.166 | 0.161 |
| 35 | 11 | 19866977 | 12418955 | 7463 | GGTCTTTTACRCACTACCTTT | 0.045 | 0.043 | 0.065 | 0.080 | 0.094 |
| 35 | 11 | 19872824 | 7950256 | 7464 | GGAAAGACTTYAGTGTCAGAA | 0.489 | 0.485 | 0.096 | 0.037 | 0.099 |
| 35 | 11 | 19884920 | 2028608 | 7465 | AGCTGTTAAARTAGAACTCCG | 0.161 | 0.157 | 0.034 | 0.021 | 0.098 |
| 35 | 11 | 19894431 | 10833192 | 7466 | ATGTTCAGCARTGCAGCCCAA | 0.180 | 0.171 | 0.062 | 0.074 | 0.050 |
| 35 | 11 | 19906067 | 12223116 | 7467 | ACAAGGCCTGRTCATGTTTCC | 0.027 | 0.027 | 0.036 | 0.057 | 0.022 |
| 35 | 11 | 19912076 | 1372989 | 7468 | CAGGAATGAARAGCATGCCCG | 0.206 | 0.209 | 0.114 | 0.105 | 0.019 |
| 35 | 11 | 19918239 | 10833202 | 7469 | CTTCAGATAARGTTGCTTTTC | 0.362 | 0.345 | 0.111 | 0.041 | 0.023 |
| 35 | 11 | 19929891 | 11025337 | 7470 | ACCTTTGCTTRAAAGCACCGA | 0.618 | 0.597 | 0.223 | 0.028 | 0.015 |
| 35 | 11 | 19944988 | 2403547 | 7471 | ACTCTCTCACRTTTCCAACTT | 0.173 | 0.174 | 0.171 | 0.055 | 0.023 |
| 35 | 11 | 19953560 | 10833211 | 7472 | AATTAAAATGYTCCTGGACTT | 0.420 | 0.434 | 0.090 | 0.066 | 0.009 |
| 35 | 11 | 19959591 | 10833212 | 7473 | ATTACCCAACYGTGTGTGTTG | 0.002 | 0.002 | 0.071 | 0.051 | 0.012 |
| 35 | 11 | 19966633 | 1442715 | 7474 | TCCACCTTTGWGCCAGGAAAC | 0.055 | 0.056 | 0.066 | 0.044 | 0.018 |
| 35 | 11 | 19973135 | 7111951 | 7475 | GAAGGCACACRTTATTTATTT | 0.449 | 0.467 | 0.015 | 0.013 | 0.072 |
| 35 | 11 | 19980715 | 16937344 | 7476 | CCGGTTTGGTRGAAAGGAAGA | 0.139 | 0.136 | 0.046 | 0.024 | 0.075 |
| 35 | 11 | 19988441 | 11605275 | 7477 | TTTAGAGACTYATTTACAAGA | 0.075 | 0.074 | 0.052 | 0.022 | 0.074 |
| 35 | 11 | 19995854 | 10500867 | 7478 | CTATAATTCTYGTTGAAAATA | 0.260 | 0.257 | 0.029 | 0.124 | 0.066 |
| 35 | 11 | 20006435 | 4757880 | 7479 | CTGAAACTGGRTCTCCTGGTT | 0.072 | 0.069 | 0.067 | 0.154 | 0.074 |
| 35 | 11 | 20012239 | 12276043 | 7480 | AGTAAAGAGGKTAGTTGGTTC | 0.305 | 0.289 | 0.332 | 0.119 | 0.052 |
| 35 | 11 | 20022249 | 3802799 | 7481 | TGTCTGACGAKTCCGACAAAA | 0.430 | 0.419 | 0.338 | 0.185 | 0.061 |
| 35 | 11 | 20035072 | 11025365 | 7482 | TAACACAGCCRTAAAGCTCTA | 1.041 | 1.020 | 0.435 | 0.220 | 0.136 |
| 35 | 11 | 20048972 | 11025369 | 7483 | AACCCTTTTGYTTAGCAGAAA | 0.269 | 0.251 | 0.538 | 0.213 | 0.142 |
| 35 | 11 | 20056025 | 12146551 | 7484 | ACTTCATTCCYGCTCCTCCTG | 0.336 | 0.332 | 0.436 | 0.217 | 0.540 |
| 35 | 11 | 20062960 | 10833240 | 7485 | TTTAAACTTTYTGTATAGCAA | 0.544 | 0.513 | 0.143 | 0.377 | 0.538 |
| 35 | 11 | 20069068 | 1837971 | 7486 | TGAAGCTGACRGATATCCGCT | 0.200 | 0.200 | 0.114 | 0.414 | 0.470 |
| 35 | 11 | 20076082 | 2289561 | 7487 | GCTGCAATTAYCTAACAAAAG | 0.238 | 0.226 | 0.288 | 0.778 | 0.600 |
| 35 | 11 | 20089989 | 4757893 | 7488 | ACTCAGTTGARGACCAGAGAT | 0.107 | 0.109 | 0.305 | 0.698 | 0.637 |
| 35 | 11 | 20098415 | 2246192 | 7489 | CCCTGCTTTARTCTCAGCAAA | 0.900 | 0.963 | 1.169 | 0.596 | 0.726 |
| 35 | 11 | 20116159 | 1520905 | 7490 | CCATCTACCAYAAAGCAGCTC | 0.576 | 0.551 | 1.120 | 0.612 | 1.067 |
| 35 | 11 | 20122076 | 1442717 | 7491 | ATCCTTTCACKCAGGGCTGAG | 2.092 | 2.041 | 1.093 | 0.709 | 0.974 |
| 35 | 11 | 20139355 | 10833258 | 7492 | AAAAGTAAAASGAGTCTCCTT | 0.086 | 0.086 | 0.892 | 0.939 | 0.999 |
| 35 | 11 | 20153249 | 16905792 | 7493 | AGAAAGCAAGRGCCAGTCAGG | 0.030 | 0.032 | 0.823 | 1.843 | 1.066 |
| 35 | 11 | 20159301 | 10833260 | 7494 | TAGGTGTCCTYGTAGGTATTA | 0.572 | 0.561 | 0.236 | 1.403 | 1.067 |
| 35 | 11 | 20168040 | 17232465 | 7495 | AGAAACACATRGTTCAACCAA | 0.416 | 0.415 | 1.155 | 1.297 | 1.291 |
| 35 | 11 | 20178333 | — | 7496 | GTGTATTTGGYGTAAGAAGTT | 0.743 | 0.751 | 1.227 | 0.701 | 1.238 |
| 35 | 11 | 20187177 | 17298222 | 7497 | TTCAGGAGCAYTGCAACACAA | 2.141 | 2.195 | 1.118 | 0.759 | 0.981 |
| 35 | 11 | 20193972 | 11025445 | 7498 | TCATGCCAGARCTTTAGAGCC | 0.086 | 0.084 | 1.302 | 1.025 | 1.004 |
| 35 | 11 | 20199867 | 1356981 | 7499 | TGTAATAATARGTCTAATCAA | 0.413 | 0.388 | 0.977 | 0.808 | 0.521 |
| 35 | 11 | 20205827 | 1520907 | 7500 | CACTGACTTAYATGCCCTTAA | 0.616 | 0.603 | 0.248 | 0.790 | 1.444 |
| 35 | 11 | 20219977 | 1402377 | 7501 | TTTTATTAAGWTATGCCTTGG | 0.193 | 0.202 | 0.250 | 0.752 | 1.455 |
| 35 | 11 | 20225966 | 1520896 | 7502 | TTGTAAATCCRATGACTATTT | 0.621 | 0.620 | 0.255 | 0.215 | 1.342 |
| 35 | 11 | 20233015 | 10833271 | 7503 | CTGATATGAARCAATTCATCA | 0.058 | 0.054 | 0.303 | 1.186 | 1.332 |
| 35 | 11 | 20245613 | 4757917 | 7504 | GCCCTACCACRTGTGCAGGTT | 0.369 | 0.363 | 0.324 | 1.099 | 1.336 |
| 35 | 11 | 20256439 | 2403577 | 7505 | CTGTTATTGCYTTTCACTGTA | 0.680 | 0.698 | 1.463 | 0.962 | 0.784 |
| 35 | 11 | 20263795 | 17232549 | 7506 | GATTCCATCCYGTTGACTTTG | 0.340 | 0.336 | 1.492 | 1.020 | 0.813 |
| 35 | 11 | 20269195 | 12804805 | 7507 | ACCTGAGCCCSAGATTTTGGT | 3.032 | 2.714 | 1.370 | 1.097 | 1.304 |
| 35 | 11 | 20275658 | 10833282 | 7508 | TTTGGCTGATRATTTTAGGCA | 0.249 | 0.250 | 1.307 | 1.121 | 1.262 |
| 35 | 11 | 20285058 | 7106374 | 7509 | AGCTTCCTCARTAGGAAAGGT | 0.427 | 0.441 | 1.671 | 1.057 | 1.782 |
| 35 | 11 | 20290488 | 16906035 | 7510 | TAACAATAGCYAATATTCGTA | 0.211 | 0.208 | 0.309 | 1.775 | 1.754 |
| 35 | 11 | 20296884 | 11025491 | 7511 | TTCCTAGGGAYTTGCTAATGT | 1.042 | 1.058 | 0.252 | 1.975 | 1.891 |
| 35 | 11 | 20308077 | — | 7512 | GCTTATGTGTSTGTGAGTACC | 0.075 | 0.074 | 1.158 | 1.317 | 1.996 |
| 35 | 11 | 20315278 | 7929060 | 7513 | AAGGGTGACTRTTGTTCTATC | 0.000 | 0.000 | 1.376 | 1.538 | 1.856 |
| 35 | 11 | 20335244 | 10833310 | 7514 | AAGATCCACTRCTTGCACAAG | 2.180 | 2.261 | 1.539 | 1.539 | 2.275 |
| 36 | 11 | 95101359 | 1944076 | 7515 | GCAGAAAAGGSTAGAATACAG | 0.026 | 0.025 | 0.063 | 0.131 | 0.214 |
| 36 | 11 | 95109994 | 12287206 | 7516 | TTCTGAGGCTYTTAGCTCATT | 0.164 | 0.161 | 0.113 | 0.152 | 0.153 |
| 36 | 11 | 95124142 | 1255149 | 7517 | CCAAATGATTRCCTTGTTGTT | 0.067 | 0.066 | 0.091 | 0.096 | 0.256 |
| 36 | 11 | 95129000 | 1569057 | 7518 | CTGGAAGTTGRAGCTTAGAAC | 1.042 | 1.012 | 0.165 | 0.135 | 0.266 |
| 36 | 11 | 95139861 | 1255167 | 7519 | ATGTATTGTTYAAATTGAGCG | 0.056 | 0.055 | 0.181 | 0.138 | 0.303 |
| 36 | 11 | 95150156 | 12418346 | 7520 | GAGGAACACTRACAAAAGCAG | 0.259 | 0.285 | 0.533 | 0.373 | 0.492 |
| 36 | 11 | 95153145 | 1255169 | 7521 | TCTTCTTTTGSAGTAACTCAA | 0.164 | 0.169 | 0.215 | 0.450 | 0.402 |
| 36 | 11 | 95174966 | 16922554 | 7522 | AGAGGAAAAAYGAAATAATAG | 1.055 | 1.091 | 0.576 | 0.576 | 0.489 |
| 36 | 11 | 95188216 | 1255171 | 7523 | CCTTCTATCTYTTTGAACGCT | 0.145 | 0.133 | 0.646 | 0.731 | 0.546 |
| 36 | 11 | 95195830 | 12421552 | 7524 | GCAGTCATACRTACACTTGAA | 0.995 | 1.000 | 0.848 | 0.819 | 0.621 |
| 36 | 11 | 95201389 | 564480 | 7525 | CACCTGACATYACATTCATTT | 0.445 | 0.443 | 1.069 | 0.930 | 0.616 |
| 36 | 11 | 95206876 | 16922618 | 7526 | GGAAAAAAAARAAATGGCAAC | 0.000 | 0.000 | 1.230 | 1.026 | 0.397 |
| 36 | 11 | 95213832 | 17229289 | 7527 | TCACAATAAASCAGGTGTTTT | 1.577 | 1.561 | 0.967 | 0.787 | 0.630 |
| 36 | 11 | 95221172 | 12418620 | 7528 | CATATCTGAGRAATAAACTCT | 0.228 | 0.240 | 0.853 | 0.740 | 0.780 |
| 36 | 11 | 95228404 | 515546 | 7529 | TTTCTTTTAASACTATTAAGT | 0.000 | 0.000 | 0.853 | 0.372 | 0.763 |
| 36 | 11 | 95238277 | 687559 | 7530 | TAAGATGCTGRGAAGACTGCA | 0.275 | 0.282 | 0.056 | 0.601 | 0.461 |
| 36 | 11 | 95254194 | 643862 | 7531 | GCTAAAATTTKGCCAAGTTCT | 0.000 | 0.000 | 0.033 | 0.715 | 0.413 |
| 36 | 11 | 95259984 | 17840711 | 7532 | GATATAAGTAWCCTCAACTTA | 0.022 | 0.021 | 0.231 | 0.198 | 0.315 |
| 36 | 11 | 95265412 | 12422059 | 7533 | CTAGGCCTATRTACTTCATCT | 0.121 | 0.123 | 0.458 | 0.143 | 0.243 |
| 36 | 11 | 95273880 | 561635 | 7534 | TTATTTCTTCYAGTTGTCCCC | 1.030 | 1.000 | 0.298 | 0.089 | 0.490 |
| 36 | 11 | 95292570 | 12417555 | 7535 | AGAAGGAATCYTTTTTAATCC | 0.808 | 0.814 | 0.293 | 0.145 | 0.179 |
| 36 | 11 | 95295504 | 16922701 | 7536 | CTTTAAGCCAMCAATTATAGT | 0.042 | 0.043 | 0.273 | 0.103 | 0.168 |
| 36 | 11 | 95327557 | 11021358 | 7537 | TTTCACATTCWTGGTATTTTC | 0.052 | 0.049 | 0.156 | 0.435 | 0.128 |
| 36 | 11 | 95334361 | 1940208 | 7538 | GGTCAGTTAARTTTGGAGATC | 0.002 | 0.002 | 0.025 | 0.420 | 0.127 |
| 36 | 11 | 95344073 | 16922748 | 7539 | CATATCTTTAYAGCGTTACTG | 0.590 | 0.582 | 0.406 | 0.217 | 0.164 |
| 36 | 11 | 95354964 | 16922792 | 7540 | TCAGAAGACAYGGGAAACGCA | 0.070 | 0.064 | 0.404 | 0.100 | 0.217 |
| 36 | 11 | 95362324 | 7113517 | 7541 | TGAAAATCAAYCTCTGTGTGT | 1.550 | 1.574 | 0.459 | 0.129 | 0.224 |
| 36 | 11 | 95370249 | 7110427 | 7542 | CTGCCAGACCRATGAAAACAT | 0.067 | 0.070 | 0.289 | 0.242 | 0.130 |
| 36 | 11 | 95381606 | 11021381 | 7543 | CTAAACTAACRGAGTCAACTA | 0.136 | 0.136 | 0.366 | 0.326 | 0.069 |
| 36 | 11 | 95386077 | 11021388 | 7544 | AACCCTAAGCRTTCTTTGTAT | 0.024 | 0.024 | 0.112 | 0.225 | 0.068 |
| 36 | 11 | 95410392 | 10831467 | 7545 | AGATTGATACYATGACTTCTG | 0.248 | 0.253 | 0.154 | 0.280 | 0.059 |
| 36 | 11 | 95417356 | 17233750 | 7546 | CCCTAGGAGAMACCTTAATCT | 0.709 | 0.755 | 0.135 | 0.043 | 0.100 |
| 36 | 11 | 95423237 | 7125575 | 7547 | AAGAATGAATSCTTCACAGGG | 0.337 | 0.332 | 0.220 | 0.034 | 0.063 |
| 36 | 11 | 95429607 | 1940153 | 7548 | GTATGTGTAGRCTACCATTCA | 0.139 | 0.137 | 0.186 | 0.024 | 0.085 |
| 36 | 11 | 95435937 | 10831474 | 7549 | ACATTCCTGCYGTCTCACTTC | 0.261 | 0.284 | 0.040 | 0.067 | 0.011 |
| 36 | 11 | 95448039 | 1939485 | 7550 | ATTATTGATAYTGTGCTATCC | 0.128 | 0.130 | 0.010 | 0.057 | 0.011 |
| 36 | 11 | 95453741 | 11021425 | 7551 | CATAATGCATRGGTTTGCCAG | 0.018 | 0.018 | 0.042 | 0.020 | 0.041 |
| 36 | 11 | 95459675 | 10831478 | 7552 | TGACTAAAGAWCCCTCCCTAC | 0.022 | 0.022 | 0.025 | 0.017 | 0.049 |
| 36 | 11 | 95467387 | 6483478 | 7553 | TCATTCACAAMCTAGCAATCA | 0.482 | 0.489 | 0.044 | 0.017 | 0.043 |
| 36 | 11 | 95474499 | 6483479 | 7554 | AGCAGAGCTCYATCACCGGCA | 0.153 | 0.158 | 0.081 | 0.068 | 0.523 |
| 36 | 11 | 95483812 | 4753725 | 7555 | TGCCTATCATRTATTATCAAG | 0.233 | 0.237 | 0.098 | 0.061 | 0.468 |
| 36 | 11 | 95493742 | 10765791 | 7556 | GCCTCCAAATYGCTCAAGGGA | 0.217 | 0.225 | 0.213 | 0.069 | 0.481 |
| 36 | 11 | 95500304 | 7951852 | 7557 | TTCCATCAGCRCCAAATAATT | 0.162 | 0.168 | 0.187 | 1.162 | 0.467 |
| 36 | 11 | 95511342 | 17236265 | 7558 | AATGTTGAACYCCTAATGGCT | 0.897 | 0.899 | 0.156 | 1.025 | 0.474 |
| 36 | 11 | 95520632 | 1939472 | 7559 | GGGTATTGTTYGGAGCAAAGT | 0.055 | 0.055 | 1.544 | 1.016 | 0.753 |
| 36 | 11 | 95539369 | 12575359 | 7560 | GTTTCCTAACYTGCTGCCACC | 0.088 | 0.094 | 1.759 | 1.008 | 1.018 |
| 36 | 11 | 95541663 | 2155001 | 7561 | GTGCTGGCTGSTGTGGGGATT | 4.036 | 3.386 | 1.588 | 0.988 | 0.986 |
| 36 | 11 | 95554246 | 7117004 | 7562 | TTAAAATCTCYGCCTTTCTTT | 0.086 | 0.065 | 1.511 | 1.361 | 1.273 |
| 36 | 11 | 95566406 | 4753743 | 7563 | TGTGATTCAARAAGACAAATT | 0.192 | 0.183 | 1.613 | 1.426 | 1.201 |
| 36 | 11 | 95572963 | 553652 | 7564 | ACCTTAGTCCRAACAGATGAA | 0.246 | 0.248 | 0.266 | 1.468 | 1.139 |
| 36 | 11 | 95579484 | 470141 | 7565 | TTTGTGCTTTYCAGACCTAAA | 0.183 | 0.185 | 0.635 | 1.863 | 1.296 |
| 36 | 11 | 95587058 | 10831497 | 7566 | GGTCTTTTGARGCCATGCCTA | 1.211 | 1.194 | 0.727 | 0.593 | 1.117 |
| 36 | 11 | 95597365 | 12577742 | 7567 | TAACTTTCTTYTCCTTGTGCC | 0.993 | 0.965 | 1.190 | 0.582 | 1.134 |
| 36 | 11 | 95605131 | 7109768 | 7568 | GAGAAATTGTYTGGCATAAAC | 0.400 | 0.404 | 1.254 | 0.696 | 1.170 |
| 36 | 11 | 95611450 | 10501848 | 7569 | ACTTCCTTTAYTGATATGATA | 1.222 | 1.375 | 0.636 | 0.639 | 0.334 |
| 36 | 11 | 95617867 | 485642 | 7570 | AAAAAAGCCARTGCAACTGAT | 0.146 | 0.154 | 0.431 | 0.618 | 0.392 |
| 36 | 11 | 95624336 | 4278478 | 7571 | CTGCCTGTATYGTTATTAGTA | 0.013 | 0.013 | 0.300 | 0.277 | 0.386 |
| 36 | 11 | 95630786 | 10831503 | 7572 | AGATTTGCTTMCTTGATCCTA | 0.591 | 0.563 | 0.031 | 0.248 | 0.770 |
| 36 | 11 | 95639748 | 7115578 | 7573 | ACGTTATTGARAAGTAAATCT | 0.046 | 0.048 | 0.028 | 0.227 | 0.795 |
| 36 | 11 | 95648764 | 557137 | 7574 | CATTCCTTCAKCAAGAGGGTT | 0.127 | 0.125 | 0.218 | 0.056 | 0.482 |
| 36 | 11 | 95655702 | 537756 | 7575 | CCTTAATGTAYACACTTGCAA | 0.144 | 0.135 | 0.141 | 0.390 | 0.286 |
| 36 | 11 | 95662862 | 485842 | 7576 | CTTTGTTAGCYGTTGGCAGAG | 0.882 | 0.861 | 0.157 | 0.475 | 0.263 |
| 36 | 11 | 95674082 | 11021527 | 7577 | TGCATTTTAGYCCAGTTTTTC | 0.302 | 0.296 | 0.892 | 0.310 | 0.131 |
| 36 | 11 | 95682189 | 477556 | 7578 | CAATTATGAGYAATTCATTCT | 0.211 | 0.180 | 0.939 | 0.326 | 0.104 |
| 36 | 11 | 95688991 | 494901 | 7579 | GCTGTTCCTTYATACCCTATC | 1.778 | 1.928 | 0.538 | 0.372 | 0.160 |
| 36 | 11 | 95696359 | 542284 | 7580 | AAGGAACTTAYCTGTCTTCAA | 0.288 | 0.281 | 0.477 | 0.437 | 0.098 |
| 36 | 11 | 95697893 | 4753195 | 7581 | GTCTGACATCYTCATTCCATT | 0.052 | 0.048 | 0.504 | 0.196 | 0.270 |
| 36 | 11 | 95711821 | 10501852 | 7582 | CGTGCTGAGTRACAACGGGCC | 0.135 | 0.137 | 0.047 | 0.231 | 0.278 |
| 36 | 11 | 95717420 | 12791333 | 7583 | AATACTGCAAYAGTCAGGACC | 0.221 | 0.222 | 0.009 | 0.195 | 0.731 |
| 36 | 11 | 95727253 | 10831513 | 7584 | ACTGGTTAAARAATTGAACAA | 0.339 | 0.339 | 0.028 | 0.090 | 0.522 |
| 36 | 11 | 95734731 | 4753748 | 7585 | TCACAAAGCARATAAAGAAGA | 0.032 | 0.005 | 0.024 | 0.072 | 0.613 |
| 36 | 11 | 95747709 | 6483501 | 7586 | TGTAATTTATYTACATTGCCA | 0.000 | 0.000 | 0.333 | 0.597 | 0.692 |
| 36 | 11 | 95759253 | 12273522 | 7587 | AAATTATAGCRTGTAGGATGA | 0.093 | 0.091 | 0.249 | 0.558 | 0.355 |
| 36 | 11 | 95767033 | 10765805 | 7588 | GGTTCTGTATYTGCCCCCAAA | 1.219 | 1.174 | 1.152 | 0.708 | 0.350 |
| 36 | 11 | 95777788 | — | 7589 | AATCTACTTTKTCACTAATAG | 0.133 | 0.133 | 0.914 | 0.749 | 0.358 |
| 36 | 11 | 95798914 | 10501853 | 7590 | TCTGACTGACRTAATGGATCA | 2.091 | 1.972 | 1.124 | 0.766 | 0.505 |
| 36 | 11 | 95810995 | 1451333 | 7591 | GAAATTTACCRAAAGTACAGA | 0.111 | 0.108 | 0.899 | 0.666 | 0.593 |
| 36 | 11 | 95816568 | 7935312 | 7592 | GATTTTTAGASCTCTCTTTGT | 0.724 | 0.717 | 0.886 | 0.664 | 0.565 |
| 36 | 11 | 95823847 | 7928913 | 7593 | GCACTGATAAWATTTCCTGCA | 0.491 | 0.508 | 0.281 | 0.635 | 0.607 |
| 36 | 11 | 95837900 | 7935320 | 7594 | GATTGTGACGWAAAGTCCTTG | 0.217 | 0.212 | 0.269 | 0.766 | 0.578 |
| 36 | 11 | 95842433 | 7114658 | 7595 | CCTTGGGAATKATTTCGCTTG | 0.218 | 0.212 | 0.331 | 0.315 | 0.585 |
| 36 | 11 | 95854566 | 16923596 | 7596 | CTCCTTGAGCRCTCCCTGATT | 0.061 | 0.059 | 0.363 | 0.366 | 0.510 |
| 36 | 11 | 95868655 | 618929 | 7597 | GTTTCTCAGARTGTGTCTCCA | 0.960 | 0.942 | 0.347 | 0.272 | 0.602 |
| 36 | 11 | 95882778 | 564259 | 7598 | AAAACCCTGCRGGGAAAAGAA | 0.703 | 0.687 | 0.412 | 0.165 | 0.257 |
| 36 | 11 | 95899268 | 10831534 | 7599 | CCTGTACTTGYGAAACACTTG | 0.191 | 0.188 | 0.518 | 0.281 | 0.352 |
| 36 | 11 | 95908847 | 1451329 | 7600 | AATATACAGGSAATTATAGTA | 0.322 | 0.322 | 0.187 | 0.391 | 0.285 |
| 37 | 12 | 56066088 | 4108243 | 7601 | TTACAAAAATRAAAAAAATGC | 0.117 | 0.117 | 0.367 | 0.342 | 0.640 |
| 37 | 12 | 56096521 | 537482 | 7602 | GGCTGCCCAAYAGAAACTTGC | 0.948 | 0.966 | 0.334 | 0.429 | 0.598 |
| 37 | 12 | 56102189 | 1798710 | 7603 | ATCTATCCCTSTGCACTTGCA | 0.129 | 0.136 | 0.211 | 0.605 | 0.742 |
| 37 | 12 | 56112400 | 481196 | 7604 | TCAACCCTCTRGAGAACAACA | 0.001 | 0.001 | 0.580 | 0.670 | 1.176 |
| 37 | 12 | 56145698 | 7305145 | 7605 | AGTGCCGGGARGAACCCTTGG | 0.363 | 0.352 | 0.691 | 0.504 | 1.195 |
| 37 | 12 | 56152088 | 2228226 | 7606 | TGGGGAAACASAATTCCTCAA | 1.157 | 1.228 | 0.807 | 0.772 | 1.458 |
| 37 | 12 | 56163423 | 511752 | 7607 | CTGGATGATAYAATTTCCTAA | 1.294 | 1.274 | 0.794 | 1.437 | 1.752 |
| 37 | 12 | 56177450 | 17119735 | 7608 | CAGTGACTGTRGGCTAAGAAT | 0.328 | 0.325 | 1.101 | 1.710 | 1.901 |
| 37 | 12 | 56197735 | 703835 | 7609 | AAATTTTTGGRAAAGGGTAGG | 0.057 | 0.040 | 1.861 | 1.944 | 1.901 |
| 37 | 12 | 56224348 | 775238 | 7610 | CCTACCTACAYCCTCCTGTAT | 0.984 | 0.994 | 1.792 | 2.104 | 2.040 |
| 37 | 12 | 56232777 | 11172247 | 7611 | ACCAGGTCGGSAGCTGGCTTA | 2.851 | 2.568 | 1.960 | 2.032 | 2.065 |
| 37 | 12 | 56244580 | 775322 | 7612 | GTCCCAGTGGRTGAGGGTGTG | 1.036 | 1.041 | 2.227 | 1.869 | 2.377 |
| 37 | 12 | 56254982 | 1678542 | 7613 | TGAAATGCCGSTGATAGAGAG | 2.026 | 1.950 | 2.025 | 2.100 | 2.467 |
| 37 | 12 | 56262914 | 2888334 | 7614 | GTGATAAGGTSTGGACGAAAA | 1.210 | 1.247 | 1.541 | 2.181 | 2.735 |
| 37 | 12 | 56265007 | 775251 | 7615 | GAACCCAGAGYTGATTTAAAA | 1.336 | 1.355 | 1.934 | 2.362 | 2.687 |
| 37 | 12 | 56288866 | 2306390 | 7616 | CAACCCCTCGYTCACGGAGCC | 0.657 | 0.669 | 1.711 | 2.214 | 2.713 |
| 37 | 12 | 56299442 | 2277324 | 7617 | TCCAAGCCGCRGCTCTCGCCA | 1.580 | 1.592 | 2.470 | 2.845 | 2.778 |
| 37 | 12 | 56307200 | 10083154 | 7618 | ATATACTTGTRCTTTGAAAAT | 1.432 | 1.412 | 2.789 | 2.813 | 2.687 |
| 37 | 12 | 56351715 | 2640629 | 7619 | GTGTAATAAAYAAAAGGCAAC | 3.294 | 3.322 | 4.665 | 3.153 | 2.443 |
| 37 | 12 | 56376989 | 7309600 | 7620 | GCATCCCTGCRAACAGCTTCT | 0.774 | 0.732 | 3.474 | 3.332 | 2.433 |
| 37 | 12 | 56393103 | 1689590 | 7621 | AGGGCAATATRTAGGTTTGTT | 2.832 | 2.761 | 3.687 | 3.534 | 2.381 |
| 37 | 12 | 56404515 | 2269720 | 7622 | CTGCAGCTCAYGGAGGCAGCA | 1.263 | 1.282 | 2.474 | 2.801 | 2.355 |
| 37 | 12 | 56415927 | 238527 | 7623 | CATTCCACAGKAGAGAAACTG | 0.546 | 0.528 | 2.012 | 2.408 | 2.345 |
| 37 | 12 | 56444371 | 8176346 | 7624 | ATCCACTAGTYGCTTCCCCAG | 0.251 | 0.244 | 0.687 | 1.638 | 2.258 |
| 37 | 12 | 56451352 | 10877013 | 7625 | TTGGTCACACYATTGCAGAAG | 0.128 | 0.120 | 0.366 | 1.364 | 1.941 |
| 37 | 12 | 56459196 | 10877019 | 7626 | GCTGCCAAGAYTTTGTCCTCT | 0.761 | 0.785 | 0.365 | 0.548 | 1.685 |
| 37 | 12 | 56466683 | 724834 | 7627 | TTTTAGAGGASGTTTGGGGGC | 0.503 | 0.524 | 0.287 | 0.357 | 1.129 |
| 37 | 12 | 56474411 | 7976556 | 7628 | CTACCCCTTTYTACCTTTTGG | 0.518 | 0.523 | 0.455 | 0.381 | 1.079 |
| 37 | 12 | 56483069 | 1875124 | 7629 | GATTTTTCCARAGCAAGGTGG | 0.007 | 0.007 | 0.393 | 0.361 | 0.510 |
| 37 | 12 | 56488686 | 2277326 | 7630 | TATGAAGCCAMATGATATAAG | 0.557 | 0.540 | 0.411 | 0.462 | 0.308 |
| 37 | 12 | 56494431 | 1021469 | 7631 | CAGCCACAGGRTTAACGTGGC | 0.613 | 0.642 | 0.276 | 0.360 | 0.330 |
| 37 | 12 | 56508939 | 4760332 | 7632 | AAATGCCTTGMATGAGTATGG | 0.568 | 0.579 | 0.460 | 0.308 | 0.525 |
| 37 | 12 | 56514855 | 7489290 | 7633 | AGGCCCACAGSGATTTCCTTC | 0.098 | 0.100 | 0.439 | 0.253 | 0.617 |
| 37 | 12 | 56521865 | 4760334 | 7634 | TATGTAAAGARGGAGGACATT | 0.517 | 0.500 | 0.321 | 0.446 | 1.029 |
| 37 | 12 | 56548790 | 7312623 | 7635 | GAGAGAAGGGKTCAGGGGCCC | 0.000 | 0.000 | 0.195 | 0.648 | 0.934 |
| 37 | 12 | 56566859 | 11172369 | 7636 | ACTGAGGGTTYTCAAGGCCAC | 0.329 | 0.340 | 0.408 | 0.588 | 0.924 |
| 37 | 12 | 56573897 | 7971877 | 7637 | TGGGAGGGAAKAATGTCCCTG | 0.362 | 0.327 | 0.717 | 1.313 | 1.260 |
| 37 | 12 | 56588525 | 4630335 | 7638 | AGTAATTGAARGCCTTAGGTT | 0.647 | 0.633 | 0.717 | 1.339 | 1.216 |
| 37 | 12 | 56589754 | — | 7639 | AATTTCCTTTRTAAAGAAGAC | 1.188 | 1.117 | 1.843 | 1.242 | 1.123 |
| 37 | 12 | 56612866 | 17120198 | 7640 | ATAATGCTTAYTCTTGATGTT | 0.000 | 0.000 | 1.700 | 1.490 | 1.047 |
| 37 | 12 | 56621010 | 4760346 | 7641 | AGTAGGGTAARCGAATGGGTA | 2.339 | 2.146 | 1.625 | 1.512 | 1.176 |
| 37 | 12 | 56627592 | 7132343 | 7642 | TAGGATTGGARGGAAATGTAA | 0.042 | 0.043 | 1.690 | 1.435 | 1.184 |
| 37 | 12 | 56635863 | 17120058 | 7643 | ATGTTAGTGARTATCTCCAGC | 0.585 | 0.568 | 1.482 | 1.288 | 1.181 |
| 38 | 12 | 81345981 | 1874058 | 7644 | GCTCCTTGATKACCATTTTTT | 0.000 | 0.000 | 1.164 | 0.588 | 0.322 |
| 38 | 12 | 81358422 | 2731295 | 7645 | TGTACCAGGCRATGCTTCCAT | 1.505 | 1.445 | 1.014 | 0.588 | 0.473 |
| 38 | 12 | 81366314 | 1494024 | 7646 | CCATCAAAGASAATAATCACT | 0.131 | 0.129 | 0.412 | 0.528 | 0.488 |
| 38 | 12 | 81372465 | 17009623 | 7647 | AGAAGAAACTRCCTTAAGTAT | 0.186 | 0.186 | 0.412 | 0.534 | 0.798 |
| 38 | 12 | 81377879 | 7133956 | 7648 | TTAAATGTGGRATGCAAGCAT | 0.047 | 0.048 | 0.006 | 0.527 | 0.811 |
| 38 | 12 | 81382578 | 7967097 | 7649 | AATTGCAGTAYTACTTTTGTC | 0.000 | 0.000 | 0.054 | 0.460 | 1.001 |
| 38 | 12 | 81413513 | 17776375 | 7650 | CTTGTTGGTCYCATTGCACTG | 0.026 | 0.025 | 0.411 | 0.374 | 1.029 |
| 38 | 12 | 81423611 | 1565801 | 7651 | CATTATCTGAYAACCAGTAGA | 0.516 | 0.489 | 0.502 | 0.520 | 0.742 |
| 38 | 12 | 81429920 | 10506875 | 7652 | TGAACATAGTRTCTAGATCTA | 1.272 | 1.189 | 0.746 | 0.732 | 0.683 |
| 38 | 12 | 81434488 | 4882510 | 7653 | CACATTGGTCYCAAATTTCAA | 0.241 | 0.254 | 0.936 | 0.861 | 0.441 |
| 38 | 12 | 81456620 | 12580555 | 7654 | GGAATACAGTRTGCCCTTTCA | 1.192 | 1.118 | 1.125 | 0.766 | 0.481 |
| 38 | 12 | 81462973 | 11834171 | 7655 | ACCCAAGAGTKAGGACTAGTA | 0.615 | 0.609 | 0.967 | 0.833 | 0.562 |
| 38 | 12 | 81469479 | 17720596 | 7656 | ACTGTTGTTCMTCATAGTATC | 0.992 | 0.930 | 0.885 | 0.828 | 0.712 |
| 38 | 12 | 81474730 | 12582370 | 7657 | AAAGCAAATTKGTTTTTTCCA | 0.480 | 0.537 | 0.532 | 0.604 | 0.661 |
| 38 | 12 | 81489232 | 17777291 | 7658 | ATAAGGACCAKAAAGGAATGA | 0.204 | 0.200 | 0.449 | 0.687 | 0.663 |
| 38 | 12 | 81496639 | 17009739 | 7659 | TGAATACATCYGAAGTAAAGA | 0.267 | 0.269 | 0.238 | 0.585 | 0.622 |
| 38 | 12 | 81502516 | 11115314 | 7660 | ATTCCAGAACKTTTGTCCTTA | 0.425 | 0.419 | 0.266 | 0.499 | 0.580 |
| 38 | 12 | 81508836 | 12821789 | 7661 | AGAGATTTGAYATACATTCAA | 0.316 | 0.320 | 0.483 | 0.275 | 0.683 |
| 38 | 12 | 81521180 | 17009794 | 7662 | GAGTAGCTCTWCATTTGCCAA | 0.593 | 0.604 | 0.493 | 0.209 | 0.579 |
| 38 | 12 | 81548422 | 10506877 | 7663 | GATAAAAGTTMCTACTGAGGA | 0.795 | 0.816 | 0.341 | 0.372 | 0.504 |
| 38 | 12 | 81561761 | 17722367 | 7664 | ATCTGACTTCYAGCATTCAGT | 0.360 | 0.315 | 0.278 | 0.494 | 0.330 |
| 38 | 12 | 81570312 | 7316112 | 7665 | GTATTCCACARAAGAATAAAG | 0.001 | 0.001 | 0.414 | 0.561 | 0.245 |
| 38 | 12 | 81579199 | 11115342 | 7666 | AGATGTGGATYTTTAGTTAAT | 0.190 | 0.190 | 0.390 | 0.535 | 0.263 |
| 38 | 12 | 81592425 | 10862496 | 7667 | TCAAATGATGMAGAAGCTTAA | 0.976 | 0.954 | 0.536 | 0.398 | 0.341 |
| 38 | 12 | 81602869 | 11115353 | 7668 | AGGATTTGTAKTGCCAGTGGT | 0.761 | 0.797 | 0.640 | 0.204 | 0.342 |
| 38 | 12 | 81610863 | 4620789 | 7669 | AGGATAATGTKCTAGATAGCT | 0.677 | 0.683 | 0.607 | 0.204 | 0.284 |
| 38 | 12 | 81617342 | 17009899 | 7670 | TTACATTTAAYGACAGGTTTT | 0.000 | 0.000 | 0.249 | 0.387 | 0.348 |
| 38 | 12 | 81627538 | 10506879 | 7671 | CAAATGTTTCYGCGTAGGTTG | 0.127 | 0.120 | 0.125 | 0.460 | 0.232 |
| 38 | 12 | 81642625 | 7972064 | 7672 | TTGGCACTGTSCATGTTTTTG | 0.069 | 0.069 | 0.141 | 0.211 | 0.255 |
| 38 | 12 | 81659668 | 10506880 | 7673 | TGCTATCCAGRACTGTCCACT | 0.311 | 0.313 | 0.174 | 0.250 | 0.304 |
| 38 | 12 | 81668323 | 7312288 | 7674 | GCACCATGGCRCTTAACTGTA | 0.736 | 0.719 | 0.154 | 0.151 | 0.325 |
| 38 | 12 | 81678985 | 6539689 | 7675 | ATTTTCTACCYTTTCTAGACT | 0.000 | 0.000 | 0.499 | 0.204 | 0.170 |
| 38 | 12 | 81685472 | 12368738 | 7676 | GTGAATTGCASGTGTAGGAGG | 0.052 | 0.052 | 0.435 | 0.236 | 0.111 |
| 38 | 12 | 81691248 | 7309393 | 7677 | GTGAATCGCTKGAATCCGAAG | 0.900 | 0.880 | 0.340 | 0.289 | 0.107 |
| 38 | 12 | 81692856 | 17009995 | 7678 | TTAGGGGAAARGTATATATAT | 0.192 | 0.194 | 0.269 | 0.223 | 0.082 |
| 38 | 12 | 81710613 | 1858059 | 7679 | CAAAGCTCCAWGGGAGAATAC | 0.414 | 0.416 | 0.326 | 0.143 | 0.080 |
| 38 | 12 | 81719954 | 17041817 | 7680 | TTATTTTTACRGAAAAACCCC | 0.291 | 0.279 | 0.081 | 0.180 | 0.111 |
| 38 | 12 | 81729819 | 17010029 | 7681 | TCACAAAATGYGTAGTATATT | 0.338 | 0.328 | 0.118 | 0.156 | 0.125 |
| 38 | 12 | 81741749 | 9651971 | 7682 | AAGCCTTACARTATCCCTTCT | 0.063 | 0.063 | 0.158 | 0.041 | 0.081 |
| 38 | 12 | 81752531 | 12300038 | 7683 | TCTATAGAGGRTAGTAAGTCC | 0.326 | 0.328 | 0.098 | 0.058 | 0.063 |
| 38 | 12 | 81766686 | 17010069 | 7684 | TACTGTTTGAMATAGTTTATC | 0.572 | 0.541 | 0.050 | 0.058 | 0.061 |
| 38 | 12 | 81776529 | 1703109 | 7685 | TAAATAAAGCSTCTAGTACCT | 0.005 | 0.005 | 0.095 | 0.066 | 0.038 |
| 38 | 12 | 81792738 | 17010106 | 7686 | AGAGCCCTTTMTGTCAAAGTC | 0.151 | 0.145 | 0.128 | 0.047 | 0.130 |
| 38 | 12 | 81801101 | 17726837 | 7687 | GGCACCATTGRTCTCTGTTAC | 0.357 | 0.359 | 0.086 | 0.047 | 0.146 |
| 38 | 12 | 81808589 | 9308329 | 7688 | GTCGTTTAATRTTTGGGGCTC | 0.429 | 0.424 | 0.119 | 0.091 | 0.305 |
| 38 | 12 | 81819344 | 17010128 | 7689 | ATGTCTGCCCYGGGCCACACT | 0.369 | 0.348 | 0.109 | 0.213 | 0.418 |
| 38 | 12 | 81829259 | 10778916 | 7690 | AATTCCTAGCRAAAAATCTGT | 0.150 | 0.155 | 0.192 | 0.350 | 0.498 |
| 38 | 12 | 81840419 | — | 7691 | TGGTGTCATARTTATGTAAAT | 0.214 | 0.089 | 0.495 | 0.681 | 0.528 |
| 38 | 12 | 81849545 | 12297736 | 7692 | GCTAGTGCATRTTAGTTACTT | 0.672 | 0.657 | 0.597 | 0.846 | 0.450 |
| 38 | 12 | 81857607 | 17010268 | 7693 | GCCCAAATTTWTCCAGAACTT | 1.150 | 1.142 | 1.034 | 0.811 | 0.529 |
| 38 | 12 | 81869499 | 2403024 | 7694 | CCATATAACTRGATTAGTCAT | 0.535 | 0.534 | 1.318 | 0.864 | 0.736 |
| 38 | 12 | 81877247 | 10778922 | 7695 | TTTCTTTGTCKCTAGATTGTA | 1.114 | 1.101 | 1.154 | 0.893 | 0.678 |
| 38 | 12 | 81887096 | 10778923 | 7696 | TAATCCCTTCMTTTGGTCAAC | 0.859 | 0.823 | 0.942 | 0.950 | 0.774 |
| 38 | 12 | 81901994 | 1655593 | 7697 | CTCCCAGGCCRCTAGCAAAGC | 0.329 | 0.347 | 0.782 | 1.052 | 0.721 |
| 38 | 12 | 81912723 | 1682605 | 7698 | GTAACAAGAGYTCTTTTAAGT | 0.000 | 0.000 | 0.374 | 0.715 | 0.728 |
| 38 | 12 | 81919181 | 10862550 | 7699 | ATTGAAATGAYTTTTGGCTAC | 0.240 | 0.243 | 0.416 | 0.807 | 0.962 |
| 38 | 12 | 81925847 | 11115552 | 7700 | TTGTCTTTACMATGGTCTGTG | 0.324 | 0.332 | 0.357 | 0.508 | 0.825 |
| 38 | 12 | 81931444 | 12301199 | 7701 | TTAATTTATAYTTGCAGCAAT | 0.901 | 0.962 | 0.539 | 0.297 | 0.735 |
| 38 | 12 | 81937271 | 12315833 | 7702 | AAGAAGGGAASACTGGCCTTT | 0.193 | 0.198 | 0.502 | 0.578 | 1.112 |
| 38 | 12 | 81946596 | 12814295 | 7703 | TTGAGGTACAKTTGAAAGAGT | 0.853 | 0.853 | 0.434 | 0.427 | 0.829 |
| 38 | 12 | 81953431 | 12833644 | 7704 | ATATAATCTTYCTAGATATGG | 0.182 | 0.181 | 0.565 | 0.542 | 2.029 |
| 38 | 12 | 81961580 | 10778937 | 7705 | ATTTTATATAYGAAGAATTCC | 0.132 | 0.137 | 0.472 | 1.074 | 2.019 |
| 38 | 12 | 81987033 | 10862580 | 7706 | ATCACTCTCCYGAATTAAGTT | 1.268 | 1.278 | 0.374 | 0.767 | 1.909 |
| 38 | 12 | 81996914 | 10506890 | 7707 | TCTGATCAACRTAAAATTAGG | 0.005 | 0.005 | 1.281 | 2.771 | 1.958 |
| 38 | 12 | 82007514 | 11115596 | 7708 | CCCATTTAATKTTTAAACTAG | 0.658 | 0.636 | 1.276 | 2.679 | 1.950 |
| 38 | 12 | 82019533 | 1543171 | 7709 | TGTCATCTGTKTCTGATTTGT | 1.822 | 1.835 | 3.156 | 2.661 | 2.563 |
| 38 | 12 | 82029933 | 2292827 | 7710 | CTATAAATTARGTAGAGAACA | 0.087 | 0.086 | 3.869 | 3.088 | 3.099 |
| 38 | 12 | 82038186 | 12146710 | 7711 | TTAGGGGTTTMAATTTCTTCT | 4.300 | 2.067 | 3.117 | 2.572 | 3.337 |
| 38 | 12 | 82046326 | 11115618 | 7712 | AAAAATAACAWGTTGAAGTAA | 0.562 | 0.562 | 2.139 | 3.481 | 3.130 |
| 38 | 12 | 82054366 | 17010700 | 7713 | GCCACCTCACRCAGAATTGTG | 0.160 | 0.168 | 2.283 | 3.298 | 2.824 |
| 38 | 12 | 82062071 | 17010710 | 7714 | CCTGCTTACCMAAAACTGCAC | 0.301 | 0.296 | 1.297 | 3.229 | 2.362 |
| 38 | 12 | 82075135 | 12301625 | 7715 | ACAATGTGCCYGTTCAGATAT | 0.364 | 0.388 | 1.417 | 2.955 | 2.519 |
| 38 | 12 | 82101670 | 7296128 | 7716 | TGCTGTGAATRTTCTATGAGC | 2.323 | 2.256 | 1.880 | 1.132 | 2.370 |
| 38 | 12 | 82106660 | 2037258 | 7717 | CTGTGCCAGGMAGTTTGGTAA | 0.710 | 0.690 | 1.842 | 0.854 | 1.918 |
| 38 | 12 | 82110836 | 2037257 | 7718 | AGTTTCCTTAYATATAGCAAA | 0.881 | 0.851 | 1.396 | 1.046 | 1.813 |
| 38 | 12 | 82132886 | 6539713 | 7719 | ATTAAATTTGYTATTACTGCT | 0.480 | 0.484 | 0.437 | 1.070 | 0.712 |
| 38 | 12 | 82145316 | 7311826 | 7720 | TTTGGAAAATKTGCAGGCCTA | 0.209 | 0.211 | 0.459 | 1.130 | 0.595 |
| 38 | 12 | 82157291 | 7296363 | 7721 | AAGTTGGTAAMAAAAAAACAA | 0.044 | 0.042 | 0.299 | 0.488 | 0.652 |
| 38 | 12 | 82166950 | 6539716 | 7722 | TTTTGTCTTTKTATGGTTTCT | 0.773 | 0.770 | 0.422 | 0.326 | 0.618 |
| 38 | 12 | 82180501 | 6539722 | 7723 | AAAAGATACCYAAAAGTGCAG | 0.397 | 0.393 | 0.330 | 0.164 | 0.591 |
| 38 | 12 | 82195147 | 4882445 | 7724 | GCCTTCAGAGKCATGAATGAG | 0.869 | 0.854 | 0.329 | 0.157 | 0.201 |
| 38 | 12 | 82206913 | 6419410 | 7725 | TTATTCAGTCWTTGACAGTGT | 0.006 | 0.006 | 0.122 | 0.134 | 0.211 |
| 38 | 12 | 82224121 | 7485518 | 7726 | GAACAAGAACMCTTCAATAAA | 0.067 | 0.068 | 0.141 | 0.159 | 0.113 |
| 38 | 12 | 82233165 | 4882378 | 7727 | TTAATTTCTTKAGGAGAAGCA | 0.051 | 0.054 | 0.016 | 0.064 | 0.249 |
| 38 | 12 | 82249263 | 6539740 | 7728 | CAGAATTAATKTCCTTGTGCC | 0.471 | 0.460 | 0.043 | 0.105 | 0.754 |
| 38 | 12 | 82263926 | 4471519 | 7729 | CAGAAAATTARACTCATTGGA | 0.181 | 0.179 | 0.053 | 0.024 | 0.909 |
| 38 | 12 | 82268637 | 4346039 | 7730 | ATTTATGTTCRGAAATAACAT | 0.251 | 0.242 | 0.228 | 0.272 | 0.749 |
| 39 | 14 | 49608345 | 2103889 | 7731 | TGCTGCTGTAYACTTTTTCAT | 0.722 | 0.757 | 0.500 | 0.542 | 0.657 |
| 39 | 14 | 49617560 | 17122058 | 7732 | ACTTAACCTTRGCATAGTAAT | 0.193 | 0.187 | 0.372 | 1.211 | 0.636 |
| 39 | 14 | 49630695 | 7141809 | 7733 | GCTGCCTTTARGATCAACCTA | 0.034 | 0.034 | 0.140 | 0.730 | 0.692 |
| 39 | 14 | 49638176 | 9944008 | 7734 | AGCTTCGTATRAAAAAAAACA | 0.217 | 0.193 | 0.618 | 0.598 | 0.584 |
| 39 | 14 | 49645558 | 10844 | 7735 | ATCACATAGAYATGTATGGTG | 0.000 | 0.000 | 0.572 | 0.319 | 0.711 |
| 39 | 14 | 49654998 | 2227276 | 7736 | AGCATCGACGYGGTACCCTTG | 1.894 | 1.887 | 0.613 | 0.253 | 0.436 |
| 39 | 14 | 49666612 | 3759597 | 7737 | AAAAATTAACYCAGAACCTTC | 0.055 | 0.057 | 0.565 | 0.309 | 0.465 |
| 39 | 14 | 49673621 | 12896468 | 7738 | AACATTCATTYAGCATAAACA | 0.095 | 0.096 | 0.541 | 0.479 | 0.314 |
| 39 | 14 | 49686628 | 8192699 | 7739 | GGCTTACTCCRTTTGCCTGAG | 0.081 | 0.081 | 0.039 | 0.473 | 0.322 |
| 39 | 14 | 49692566 | 13379306 | 7740 | CAACAGTAATMAAGGAAACTT | 0.452 | 0.444 | 0.217 | 0.516 | 0.462 |
| 39 | 14 | 49703571 | 17122240 | 7741 | ATTTCTTTACRTGCTTTTTTA | 0.000 | 0.000 | 0.272 | 0.115 | 0.500 |
| 39 | 14 | 49720888 | 6572646 | 7742 | CTAAGAATATYCTGAGTGATA | 0.618 | 0.621 | 0.432 | 0.267 | 0.520 |
| 39 | 14 | 49728687 | 7160215 | 7743 | AGTAATACTAMCATGAAAGAG | 0.108 | 0.105 | 0.307 | 0.478 | 0.709 |
| 39 | 14 | 49736977 | 12896404 | 7744 | TGGCCTACTAYATAGGAATTA | 0.643 | 0.630 | 0.412 | 0.524 | 0.604 |
| 39 | 14 | 49740883 | 12888783 | 7745 | TGAACCTTTARAAAAAAGTTA | 0.082 | 0.083 | 0.549 | 0.463 | 0.854 |
| 39 | 14 | 49752478 | 1569898 | 7746 | CTTCATTGTGYTGCCAAGATT | 0.783 | 0.760 | 0.633 | 0.742 | 1.038 |
| 39 | 14 | 49764449 | 2144575 | 7747 | TTAATTTCTAYTGACTATTTT | 1.055 | 1.009 | 0.477 | 0.987 | 1.275 |
| 39 | 14 | 49769618 | 1955926 | 7748 | AACTACTGTGMTGTCAAACTA | 0.315 | 0.296 | 1.134 | 1.410 | 1.315 |
| 39 | 14 | 49784735 | 743075 | 7749 | TGTGAAAAATYCTATCTTATA | 0.263 | 0.258 | 1.315 | 1.520 | 2.306 |
| 39 | 14 | 49802697 | 2355885 | 7750 | TCTATAAAGCYTGCCAACATC | 1.502 | 1.517 | 1.285 | 1.613 | 2.411 |
| 39 | 14 | 49806475 | 2153553 | 7751 | TGAGCAATTAYCGTGTTACTC | 1.374 | 1.429 | 1.522 | 1.568 | 2.714 |
| 39 | 14 | 49820327 | 12433038 | 7752 | AGAAAAGACARGGATGGCTTA | 1.418 | 1.387 | 1.873 | 2.456 | 2.579 |
| 39 | 14 | 49826881 | 9888567 | 7753 | ATTTGATTAAMTTTCCTAAAT | 0.906 | 0.912 | 1.796 | 2.630 | 2.556 |
| 39 | 14 | 49839467 | 2297995 | 7754 | CGCCACCAACRATGACTATAT | 1.073 | 1.073 | 3.496 | 3.191 | 2.693 |
| 39 | 14 | 49843620 | 7143204 | 7755 | ACAAAAGCTTKTGGGGAGGTA | 0.000 | 0.000 | 3.551 | 3.284 | 2.548 |
| 39 | 14 | 49857963 | 2275592 | 7756 | CAAATGATGCYGTTTGGAAAA | 4.633 | 5.267 | 3.518 | 3.026 | 2.548 |
| 39 | 14 | 49868493 | 4901016 | 7757 | CATGCAGGCTRGAAACCCTGG | 2.056 | 2.080 | 3.924 | 2.920 | 2.777 |
| 39 | 14 | 49880775 | 1465160 | 7758 | CTGCAACTGAYATACTGCAGC | 0.954 | 0.980 | 3.187 | 2.791 | 2.642 |
| 39 | 14 | 49889119 | 2153552 | 7759 | GAAGAATAGARTTGAGGGGAA | 0.086 | 0.085 | 1.486 | 2.756 | 2.661 |
| 39 | 14 | 49894589 | 11157737 | 7760 | TCTTCTTCCARTGCACACAAA | 0.338 | 0.338 | 1.176 | 2.652 | 2.358 |
| 39 | 14 | 49904329 | 9635166 | 7761 | GACTCAGATTKACTTTCTCTT | 1.348 | 1.414 | 0.824 | 1.196 | 2.294 |
| 39 | 14 | 49914981 | 7151046 | 7762 | AGGACACCTGMAGACAGTTCT | 1.413 | 1.538 | 0.994 | 0.736 | 2.317 |
| 39 | 14 | 49930907 | 11157741 | 7763 | CCCACCCATTYCAATTTATCC | 0.034 | 0.035 | 0.843 | 0.518 | 2.280 |
| 39 | 14 | 49939783 | 17717642 | 7764 | GAAAAGCCCTRCAAAGTGTAC | 0.398 | 0.364 | 0.457 | 0.558 | 1.851 |
| 39 | 14 | 49950187 | 1265880 | 7765 | CCAGCTACTTYTGCCTCCTTC | 0.074 | 0.075 | 0.082 | 0.606 | 1.257 |
| 39 | 14 | 49964206 | 12887920 | 7766 | GAGCATGCAARCAATAATAAA | 0.470 | 0.514 | 0.106 | 0.283 | 1.004 |
| 39 | 14 | 49976878 | 17718015 | 7767 | GTGTGTAAATRCCATCTTCAG | 0.270 | 0.260 | 0.088 | 0.888 | 1.281 |
| 39 | 14 | 49986853 | 17122546 | 7768 | ACCGTAAGTCKGTGATTCTTA | 0.191 | 0.131 | 0.205 | 0.931 | 2.073 |
| 39 | 14 | 49999380 | — | 7769 | ACAAATAAACYGTGTTAGTAA | 0.000 | 0.000 | 1.744 | 0.836 | 1.555 |
| 39 | 14 | 50009649 | 17791470 | 7770 | ATGTAGTTTTYTAATTGGTCT | 0.386 | 0.400 | 1.598 | 1.147 | 1.192 |
| 39 | 14 | 50011797 | 12717408 | 7771 | AAAAAATTTAYTGGCACTTTA | 3.021 | 2.690 | 1.429 | 1.720 | 1.213 |
| 39 | 14 | 50028605 | 12323534 | 7772 | AATTATTAACRTGATGCTTCG | 0.201 | 0.193 | 1.469 | 1.694 | 1.158 |
| 39 | 14 | 50035195 | 17791680 | 7773 | TAAGTTTACARCTGGAATTTC | 0.080 | 0.081 | 2.173 | 1.734 | 1.093 |
| 39 | 14 | 50046648 | 2180494 | 7774 | TTCTCGGTTAMCAATTATTAA | 0.760 | 0.755 | 0.972 | 1.591 | 1.016 |
| 39 | 14 | 50062686 | 7160618 | 7775 | TCATCCCCAAYAAAGGCAGAG | 1.918 | 2.006 | 1.162 | 1.737 | 1.013 |
| 39 | 14 | 50074963 | 17718908 | 7776 | TTGTCTGCAARTTCAATAGCT | 0.516 | 0.533 | 1.214 | 0.590 | 0.963 |
| 39 | 14 | 50093893 | 987315 | 7777 | CATATCCTTTYCTTTTTCCAG | 0.554 | 0.546 | 1.052 | 0.581 | 0.923 |
| 39 | 14 | 50139753 | 3015452 | 7778 | TTTTCACCTAYAGCATGTTTA | 0.356 | 0.343 | 0.252 | 0.650 | 0.929 |
| 39 | 14 | 50153985 | 17122693 | 7779 | CAATCACTTAYCAGAATCAGG | 0.299 | 0.303 | 0.136 | 0.468 | 0.391 |
| 39 | 14 | 50164151 | 17122701 | 7780 | TTTGGATATAYGTAGTTTTCT | 0.044 | 0.046 | 0.078 | 0.161 | 0.419 |
| 39 | 14 | 50174491 | 17122714 | 7781 | ATCAAGAGTAYGATTATGGGT | 0.153 | 0.147 | 0.052 | 0.152 | 0.439 |
| 40 | 14 | 80247029 | 17111027 | 7782 | AAAAATTATCRTCTGAAATCA | 0.012 | 0.012 | 0.303 | 0.298 | 0.268 |
| 40 | 14 | 80254648 | 17111035 | 7783 | CAGTGAGAATYTGGAACACAG | 0.140 | 0.142 | 0.286 | 0.567 | 0.230 |
| 40 | 14 | 80273661 | 327456 | 7784 | TTGAACTTCTRAACAAGCTGG | 0.302 | 0.300 | 0.128 | 0.441 | 0.327 |
| 40 | 14 | 80288710 | 327446 | 7785 | GGGCCTAATAYTTCTCACACA | 0.095 | 0.094 | 0.491 | 0.468 | 0.326 |
| 40 | 14 | 80294079 | 2590480 | 7786 | CACAGTATTTYCTTCCTCTTC | 0.861 | 0.854 | 0.521 | 0.195 | 0.346 |
| 40 | 14 | 80299870 | 17541411 | 7787 | GATTTAAACAYTTTTTGAAGA | 1.062 | 1.103 | 0.495 | 0.341 | 0.308 |
| 40 | 14 | 80307745 | 327473 | 7788 | ATAGCCTTAGMTGTCTCATAA | 0.175 | 0.177 | 0.555 | 0.386 | 0.400 |
| 40 | 14 | 80316672 | 17613911 | 7789 | CAGAAGATCCRCAGAATGCTT | 0.229 | 0.225 | 0.443 | 0.330 | 0.238 |
| 40 | 14 | 80325147 | 17614081 | 7790 | TGATATTTCTRTGGAAGTTTT | 0.270 | 0.263 | 0.163 | 0.415 | 0.352 |
| 40 | 14 | 80331771 | 327428 | 7791 | AAGGGCCATTRTGACAAAAAG | 0.583 | 0.545 | 0.141 | 0.371 | 0.455 |
| 40 | 14 | 80353610 | 2371417 | 7792 | ATCTAATAGCRATATCAGTAA | 0.350 | 0.320 | 0.208 | 0.215 | 0.633 |
| 40 | 14 | 80365281 | 17111151 | 7793 | GAAAATCAGAMGTGTCACTAA | 0.130 | 0.133 | 0.366 | 0.314 | 0.629 |
| 40 | 14 | 80376971 | 7142753 | 7794 | TGGATATGAGKTACACAAAAT | 0.425 | 0.422 | 0.312 | 0.439 | 0.474 |
| 40 | 14 | 80381905 | 11159475 | 7795 | TTACTATGCAYTCAGTAGTTG | 0.715 | 0.708 | 0.438 | 0.756 | 0.290 |
| 40 | 14 | 80393265 | — | 7796 | TAAATTTTTAYCTAGGATTTT | 0.463 | 0.449 | 0.718 | 0.578 | 0.279 |
| 40 | 14 | 80415919 | 2556611 | 7797 | CCAGATCTACRAAGCTCAAAT | 0.646 | 0.637 | 1.089 | 0.509 | 0.265 |
| 40 | 14 | 80431484 | 8010968 | 7798 | TAAAAACTACMGAAAAATCAG | 0.713 | 0.716 | 0.757 | 0.504 | 0.234 |
| 40 | 14 | 80444575 | 6574608 | 7799 | TCTATTCATTMAAGGTCAGTT | 1.274 | 1.204 | 0.601 | 0.433 | 0.185 |
| 40 | 14 | 80452982 | 4899780 | 7800 | CTCACTTAATMCTACCAACAA | 0.055 | 0.054 | 0.384 | 0.287 | 0.205 |
| 40 | 14 | 80455324 | — | 7801 | GAAGACAACTYCACCAGATAT | 0.095 | 0.095 | 0.200 | 0.215 | 0.248 |
| 40 | 14 | 80460544 | 2059719 | 7802 | AATGAAAGATSAAAGTAAATA | 0.140 | 0.138 | 0.010 | 0.127 | 0.202 |
| 40 | 14 | 80470519 | 9323695 | 7803 | GCACATAGCTRGAAATCAGAA | 0.213 | 0.211 | 0.011 | 0.087 | 0.206 |
| 40 | 14 | 80478424 | 17111256 | 7804 | AAGTAGATGTRAGGCTGTCAG | 0.179 | 0.173 | 0.021 | 0.019 | 0.206 |
| 40 | 14 | 80484907 | 2217177 | 7805 | ATTTGCCCCAYTTTAAAAGTT | 0.081 | 0.084 | 0.052 | 0.019 | 0.167 |
| 40 | 14 | 80492010 | 2239610 | 7806 | GAGGTGGCCCSAAGTGCACAA | 0.197 | 0.205 | 0.102 | 0.071 | 0.155 |
| 40 | 14 | 80499216 | 179245 | 7807 | GATATCCCAASTAGGTCAACA | 0.384 | 0.395 | 0.093 | 0.113 | 0.063 |
| 40 | 14 | 80511106 | 179259 | 7808 | AATCTTAGCARGGCACATAAT | 0.448 | 0.462 | 0.264 | 0.149 | 0.077 |
| 40 | 14 | 80519443 | 3783945 | 7809 | AGAGCTTTTTSATTTGCCATC | 0.037 | 0.038 | 0.323 | 0.231 | 0.152 |
| 40 | 14 | 80527693 | 724170 | 7810 | ATTCAACATTRAAGGAGACAT | 0.758 | 0.750 | 0.315 | 0.297 | 0.193 |
| 40 | 14 | 80540892 | 17545310 | 7811 | ACTGGAAGTCMTATTCATGGT | 0.394 | 0.390 | 0.366 | 0.287 | 0.181 |
| 40 | 14 | 80547190 | 4903965 | 7812 | AGGAACTAACRTGACTGAGTT | 0.358 | 0.371 | 0.500 | 0.409 | 0.173 |
| 40 | 14 | 80554902 | 4903967 | 7813 | TGAAAAGTTGRTCACCCTATG | 0.597 | 0.626 | 0.304 | 0.386 | 0.252 |
| 40 | 14 | 80566095 | 10143800 | 7814 | AGTTCTGCACRAAGGCCTCTT | 0.396 | 0.391 | 0.481 | 0.402 | 0.291 |
| 40 | 14 | 80570637 | 726627 | 7815 | AATTTAAGCAKAAAAAGGGTC | 0.230 | 0.231 | 0.500 | 0.237 | 0.366 |
| 40 | 14 | 80585638 | 722540 | 7816 | TCCTCAAAGCYCAGATGGTTC | 0.835 | 0.860 | 0.288 | 0.293 | 0.568 |
| 40 | 14 | 80598165 | 17111401 | 7817 | GCTTTTTAATWATGTGTTTTT | 0.390 | 0.392 | 0.192 | 0.324 | 0.648 |
| 40 | 14 | 80610085 | 7145101 | 7818 | ATATAAACCCYATTGATCTTA | 0.113 | 0.105 | 0.322 | 0.362 | 0.599 |
| 40 | 14 | 80623539 | 2284735 | 7819 | GACAAAATAARCCCAAGTGAA | 0.125 | 0.125 | 0.202 | 0.692 | 1.181 |
| 40 | 14 | 80631310 | 10143087 | 7820 | TAGCCCAGAGSTCCCAAGGCT | 0.612 | 0.622 | 0.346 | 0.718 | 1.200 |
| 40 | 14 | 80639431 | 1017141 | 7821 | TAGCAATTTGRTAATTTCTCC | 0.496 | 0.486 | 0.879 | 0.605 | 2.021 |
| 40 | 14 | 80648058 | 2888049 | 7822 | GAAGTGGTCARTTAAACTACC | 0.770 | 0.750 | 1.011 | 1.348 | 2.611 |
| 40 | 14 | 80661507 | 7158881 | 7823 | GTAAAGATGGYCCCTGTGGAA | 1.389 | 1.411 | 0.963 | 1.429 | 3.031 |
| 40 | 14 | 80668665 | 17111530 | 7824 | GTTACTTTCAYTAGACACTTA | 0.353 | 0.355 | 2.032 | 2.738 | 2.838 |
| 40 | 14 | 80677583 | 2300541 | 7825 | TGCCTGTATGKTAGTGTCCAT | 0.539 | 0.550 | 1.779 | 3.195 | 2.656 |
| 40 | 14 | 80690636 | 28441485 | 7826 | CTGTTAGGGGKAAGTTTAATG | 2.450 | 2.546 | 2.650 | 3.422 | 2.735 |
| 40 | 14 | 80699947 | 12372876 | 7827 | AATCAAAACAYGGCAAAGGGA | 0.379 | 0.363 | 3.057 | 3.127 | 2.781 |
| 40 | 14 | 80709737 | 1957546 | 7828 | AGTCACTTCAYCAAGACTGTA | 2.971 | 2.672 | 3.153 | 2.652 | 2.710 |
| 40 | 14 | 80713553 | 4899786 | 7829 | TGGCCACCAAKCAAGAGTTTG | 2.630 | 2.619 | 2.621 | 2.682 | 2.725 |
| 40 | 14 | 80727089 | 7149672 | 7830 | ATGTTACTTCRAGAAGCACTG | 2.331 | 2.193 | 2.525 | 2.682 | 2.770 |
| 40 | 14 | 80733206 | 759939 | 7831 | TACTCACTTGSCATGTTAAAT | 1.012 | 1.005 | 2.372 | 2.400 | 2.602 |
| 40 | 14 | 80741859 | 1951614 | 7832 | CTCTGCTAATYCCAGGCGAAA | 1.315 | 1.256 | 2.070 | 2.430 | 2.621 |
| 40 | 14 | 80747849 | 7148100 | 7833 | TTACACTTCTRGGTTCCCAAG | 2.122 | 1.957 | 1.781 | 2.403 | 2.705 |
| 40 | 14 | 80783439 | 7160569 | 7834 | TGGTTGTGTCYACTACAGGCA | 1.268 | 1.315 | 1.922 | 2.152 | 2.532 |
| 40 | 14 | 80789110 | 8015423 | 7835 | GAGGATGATGYTTATTGTTCC | 1.485 | 1.419 | 2.287 | 1.977 | 2.648 |
| 40 | 14 | 80797947 | 17111619 | 7836 | ATCTTCACCAMCCTCTTTGAT | 1.045 | 1.082 | 1.944 | 2.342 | 2.481 |
| 40 | 14 | 80807873 | 7161073 | 7837 | TTGTCATTCAYCTGCCATACA | 2.430 | 2.410 | 1.989 | 2.335 | 2.226 |
| 40 | 14 | 80817987 | 8010611 | 7838 | CATTAAAAAAYATCAAAAGAC | 0.178 | 0.177 | 2.453 | 2.291 | 2.059 |
| 40 | 14 | 80826232 | 2081988 | 7839 | TATTCCATTGYATGGGCACAG | 0.779 | 0.768 | 2.010 | 2.120 | 2.090 |
| 40 | 14 | 80833708 | 1885604 | 7840 | GCATGCTATAYGATTTAAATT | 1.724 | 1.753 | 1.141 | 1.795 | 2.360 |
| 40 | 14 | 80834880 | 17111691 | 7841 | TGTTGCTCTCRGAGATTTAAG | 0.131 | 0.106 | 1.305 | 1.453 | 1.923 |
| 40 | 14 | 80848016 | 4903980 | 7842 | GTGAGCCTTAYGGTTTGGCTG | 1.240 | 1.252 | 1.100 | 0.700 | 1.627 |
| 40 | 14 | 80859232 | 8013992 | 7843 | GACTAGGCCARGAACAGAGGC | 0.876 | 0.857 | 0.412 | 1.042 | 1.211 |
| 40 | 14 | 80871291 | 7140168 | 7844 | GAGTTGGCACYAAAAATGATG | 0.010 | 0.010 | 0.419 | 0.806 | 0.923 |
| 40 | 14 | 80905656 | 3853417 | 7845 | TGTTTCAACCSTGATGGCCAC | 0.052 | 0.052 | 0.384 | 0.370 | 0.483 |
| 40 | 14 | 80915606 | — | 7846 | TATAAATTTASTTCAGTGTCA | 0.163 | 0.163 | 0.176 | 0.404 | 0.615 |
| 40 | 14 | 80923160 | 1569012 | 7847 | GGCTAAAGGAYTCAACGTGAA | 1.167 | 1.164 | 0.238 | 0.170 | 0.705 |
| 40 | 14 | 80930316 | 3844532 | 7848 | ATCTGATGTTYAAATATGAAA | 0.158 | 0.155 | 0.290 | 0.075 | 0.509 |
| 41 | 16 | 46450036 | 3903067 | 7849 | CCATTTTCACRTGCCTCTACT | 0.233 | 0.239 | 0.060 | 0.358 | 0.222 |
| 41 | 16 | 46461498 | 10492847 | 7850 | CATGCTCTTAYTTTTGCAGGT | 0.032 | 0.033 | 0.034 | 0.348 | 0.274 |
| 41 | 16 | 46467047 | 3852743 | 7851 | TGGAGTCTTCRGCTATCAGAT | 0.045 | 0.046 | 0.564 | 0.319 | 0.216 |
| 41 | 16 | 46473394 | 16945694 | 7852 | AAATTACTGCRCACTGCTGAT | 0.355 | 0.363 | 0.582 | 0.317 | 0.224 |
| 41 | 16 | 46479558 | — | 7853 | GTGGGGTAAARAATGGAATCA | 1.954 | 1.886 | 0.571 | 0.443 | 0.222 |
| 41 | 16 | 46498606 | 888339 | 7854 | CCGGTGAGCGYGGTGCTGAGA | 0.354 | 0.326 | 0.800 | 0.386 | 0.199 |
| 41 | 16 | 46515180 | — | 7855 | GGGAAACGCTSATGTGTATGG | 0.022 | 0.022 | 0.978 | 0.462 | 0.148 |
| 41 | 16 | 46517669 | 8054264 | 7856 | TGAGCTGCAARTGTGGAGACC | 0.581 | 0.575 | 0.161 | 0.549 | 0.151 |
| 41 | 16 | 46532044 | 9933191 | 7857 | CTGGGAAPAGSTGGGAGACCA | 0.565 | 0.626 | 0.166 | 0.447 | 0.198 |
| 41 | 16 | 46539255 | 7195967 | 7858 | GAAAATGATGRCTGTTTTCAT | 0.065 | 0.065 | 0.277 | 0.063 | 0.246 |
| 41 | 16 | 46563842 | — | 7859 | AGTAGATGACRATGATGACGA | 0.334 | 0.339 | 0.105 | 0.051 | 0.236 |
| 41 | 16 | 46575693 | — | 7860 | CAGGGTCTGTSTTGATGGAGA | 0.325 | 0.332 | 0.038 | 0.114 | 0.187 |
| 41 | 16 | 46589830 | 12149765 | 7861 | TTCCAGACCCRGGGTCCTACC | 0.005 | 0.005 | 0.051 | 0.094 | 0.023 |
| 41 | 16 | 46626615 | 494116 | 7862 | GGACATCAAGYTCCAAACCCT | 0.223 | 0.223 | 0.082 | 0.034 | 0.044 |
| 41 | 16 | 46636635 | 520151 | 7863 | GAATGTGGATYATTTGTTCCC | 0.156 | 0.159 | 0.104 | 0.030 | 0.776 |
| 41 | 16 | 46654645 | — | 7864 | CTGGGAATAAYCTAGTTCCTC | 0.572 | 0.586 | 0.106 | 0.020 | 0.913 |
| 41 | 16 | 46668184 | 8048367 | 7865 | CAGCTCTGGGRTAAAAGTCCA | 0.400 | 0.396 | 0.068 | 0.035 | 0.879 |
| 41 | 16 | 46679401 | 16945787 | 7866 | TCTGAGGTCTKCCAAGCTGAG | 0.017 | 0.017 | 0.076 | 1.240 | 1.010 |
| 41 | 16 | 46697131 | — | 7867 | AGGAGTAACCYTCAGGCAACC | 0.033 | 0.034 | 0.087 | 1.605 | 1.159 |
| 41 | 16 | 46709635 | 16945828 | 7868 | GAGGAACCTAKGAACGTCCGT | 0.200 | 0.200 | 1.544 | 1.725 | 1.233 |
| 41 | 16 | 46716800 | 1345425 | 7869 | TGTGAAGGTCYTTATGGAGGT | 0.572 | 0.552 | 2.389 | 1.638 | 1.766 |
| 41 | 16 | 46722278 | 12149826 | 7870 | AAAACTTCATYACATTAAACA | 4.291 | 3.568 | 2.798 | 1.601 | 1.661 |
| 41 | 16 | 46729214 | 16945857 | 7871 | CAAGGAGTGTYGAGTCAGTGG | 1.133 | 1.095 | 3.723 | 1.782 | 1.729 |
| 41 | 16 | 46736483 | 16945892 | 7872 | CACATTGCCTYTCCACAAAGC | 0.466 | 0.477 | 3.275 | 2.496 | 1.577 |
| 41 | 16 | 46748746 | 8058886 | 7873 | CAAGTTTATGRAGGAAGAATG | 0.503 | 0.511 | 0.895 | 2.636 | 1.510 |
| 41 | 16 | 46754604 | 8050306 | 7874 | GTTTCTCTTAYGATATTGTCC | 0.793 | 0.798 | 1.225 | 2.772 | 1.793 |
| 41 | 16 | 46760004 | 17821664 | 7875 | CTACGTAGGAKAAGGTTTGCC | 0.471 | 0.474 | 1.016 | 1.066 | 1.872 |
| 41 | 16 | 46768033 | 10163462 | 7876 | AGGTATCCAARGCCCAGTACC | 1.875 | 1.814 | 1.057 | 0.736 | 2.189 |
| 41 | 16 | 46775302 | 16945919 | 7877 | TCAAACCCCCMGGCTAAGGTC | 0.119 | 0.122 | 0.808 | 0.833 | 2.391 |
| 41 | 16 | 46779770 | 17822052 | 7878 | ATCACCCCCASTAGGGCATTC | 0.000 | 0.000 | 0.683 | 0.759 | 1.068 |
| 41 | 16 | 46793078 | 8050120 | 7879 | GCCTGGGTTASAGGTGTCGTC | 0.219 | 0.207 | 0.219 | 0.790 | 1.371 |
| 41 | 16 | 46799880 | 17822471 | 7880 | ACCACTCCCCRTGTTGCCGCA | 0.214 | 0.210 | 0.297 | 1.029 | 1.282 |
| 41 | 16 | 46806419 | 8047091 | 7881 | TCTCCAATAGYTTCCTTTCCT | 0.849 | 0.788 | 0.474 | 0.491 | 1.273 |
| 41 | 16 | 46815699 | 17822931 | 7882 | CTTACTGGCCYGAGTACACTG | 0.322 | 0.325 | 0.892 | 1.169 | 1.134 |
| 41 | 16 | 46822561 | 11863414 | 7883 | CGTGTGATTGYCAAGGTCCAA | 0.887 | 0.888 | 0.834 | 1.079 | 1.338 |
| 41 | 16 | 46829887 | 16946014 | 7884 | AATGGACTTTYAACTCCTTCT | 1.325 | 1.311 | 1.628 | 1.243 | 1.011 |
| 41 | 16 | 46840944 | 17750580 | 7885 | GTCTCACCAGSTACTCTGTTT | 0.042 | 0.043 | 1.488 | 1.202 | 1.061 |
| 41 | 16 | 46852072 | 16946039 | 7886 | TGCTCCAGTARTGGTTGTTTA | 2.537 | 2.384 | 1.443 | 1.427 | 0.996 |
| 41 | 16 | 46865588 | 11076561 | 7887 | TCCTCCATTARTGTAAGTGCA | 0.626 | 0.619 | 1.094 | 1.293 | 1.100 |
| 41 | 16 | 46867110 | 9931294 | 7888 | TTGATTGTGGWTGAAGCAAGA | 1.002 | 0.990 | 1.502 | 1.212 | 1.065 |
| 41 | 16 | 46891036 | — | 7889 | TATGGGAGCCRTGTAAATAAT | 0.045 | 0.047 | 0.891 | 1.028 | 1.059 |
| 41 | 16 | 46893998 | 9302757 | 7890 | CACACATAGCRTCTTTTTCAC | 2.050 | 1.950 | 0.942 | 1.156 | 1.065 |
| 41 | 16 | 46909639 | 9933211 | 7891 | TCAATTTAAARTGCATCAAAA | 0.185 | 0.175 | 0.769 | 0.815 | 0.999 |
| 41 | 16 | 46918683 | — | 7892 | AGAGCTGGGAKATACCTCTGC | 0.864 | 0.832 | 0.995 | 0.807 | 0.907 |
| 41 | 16 | 46925991 | 13338978 | 7893 | ACATCATCTTSTTGCAGTTGT | 0.389 | 0.382 | 0.566 | 0.766 | 0.944 |
| 41 | 16 | 46935615 | 8055028 | 7894 | TGTACTTCATMATTTCTGAGG | 1.282 | 1.241 | 0.603 | 0.773 | 0.689 |
| 41 | 16 | 46981974 | 17825174 | 7895 | CTCATTCATCRTGTCTTACAG | 0.034 | 0.034 | 0.598 | 0.435 | 0.687 |
| 41 | 16 | 46985312 | — | 7896 | CTGGTCATCARAGCAGTTTAA | 0.276 | 0.272 | 0.479 | 0.436 | 0.874 |
| 42 | 16 | 73578487 | 9925422 | 7897 | TGCCTGGACCRTTTCATGTTT | 0.557 | 0.559 | 0.496 | 0.801 | 1.481 |
| 42 | 16 | 73585421 | 9925758 | 7898 | TGGGCTGTTCYCTTTGCCTGG | 0.706 | 0.706 | 0.214 | 0.559 | 1.511 |
| 42 | 16 | 73603685 | 16952761 | 7899 | GTTGTAGTGTRAAAGGATCAT | 0.361 | 0.363 | 0.515 | 0.599 | 0.935 |
| 42 | 16 | 73608361 | 9925180 | 7900 | AATAAACACARTGAAGTACAG | 0.019 | 0.020 | 0.584 | 0.394 | 0.742 |
| 42 | 16 | 73626020 | 4888334 | 7901 | TCCGTTCTTTYGATTTTAACT | 0.880 | 0.856 | 0.531 | 0.584 | 0.491 |
| 42 | 16 | 73638451 | 16953733 | 7902 | ATAATCTGTCYCTTGCCATTA | 0.718 | 0.727 | 0.418 | 0.563 | 0.747 |
| 42 | 16 | 73644376 | 17684886 | 7903 | GCCACTGGGGWCATGATGTGA | 0.622 | 0.578 | 0.740 | 0.417 | 1.272 |
| 42 | 16 | 73648772 | 7196872 | 7904 | GAGTTTTTTTWAATATATTGA | 0.106 | 0.105 | 0.569 | 0.433 | 1.357 |
| 42 | 16 | 73665578 | 11149795 | 7905 | CTGTCCGTGASCGTTGCTAGA | 0.760 | 0.753 | 0.330 | 0.973 | 1.492 |
| 42 | 16 | 73696428 | 2287989 | 7906 | GGCAGAGGCCRTCAGACAGGT | 0.000 | 0.000 | 0.292 | 1.647 | 1.300 |
| 42 | 16 | 73701798 | 1043503 | 7907 | GGCCTAGCACRCTTTGATGAG | 0.237 | 0.232 | 0.953 | 1.487 | 1.646 |
| 42 | 16 | 73722875 | 7206259 | 7908 | AGAAGCATCAYGTTGCTAACA | 0.447 | 0.447 | 1.846 | 1.535 | 4.488 |
| 42 | 16 | 73761944 | 12935567 | 7909 | ATATATCCAGYTGTAAATAGC | 1.466 | 1.440 | 1.769 | 1.777 | 4.488 |
| 42 | 16 | 73762714 | 17673793 | 7910 | CAGAGACTTTRGAGGAAAAAT | 2.322 | 2.561 | 2.007 | 1.920 | 3.604 |
| 42 | 16 | 73787731 | 1559362 | 7911 | TGGTTTTGCCYGTGTGACCAA | 0.345 | 0.354 | 1.928 | 4.267 | 3.687 |
| 42 | 16 | 73789787 | 10514392 | 7912 | TCTTGCGACTRCTCCAGACGT | 0.797 | 0.804 | 1.616 | 4.488 | 4.187 |
| 42 | 16 | 73812718 | 8051363 | 7913 | TTCTGCTTCCRATGTGCCATT | 0.137 | 0.138 | 2.619 | 4.364 | 4.488 |
| 42 | 16 | 73823220 | 8062565 | 7914 | TCTGAAAACAMGGCGATCCCA | 0.865 | 0.862 | 3.503 | 4.187 | 4.488 |
| 42 | 16 | 73837879 | 28439846 | 7915 | GGTCAGAGACRGCCTCCCCAA | 4.037 | 3.761 | 3.447 | 2.832 | 4.364 |
| 42 | 16 | 73851712 | 4261573 | 7916 | TTTGATTCGAYTGATGGCTAC | 0.737 | 0.735 | 3.623 | 3.275 | 3.886 |
| 42 | 16 | 73865731 | 4888370 | 7917 | TGTGTGTTTCYCAGCCCCAGG | 0.687 | 0.706 | 3.852 | 3.091 | 2.855 |
| 42 | 16 | 73878532 | 4243112 | 7918 | CTGAGCAACCYTTCAAAGGTG | 1.299 | 1.294 | 1.176 | 3.114 | 2.189 |
| 42 | 16 | 73884883 | 17605723 | 7919 | AACAAGCTGCRAAGGGGCCTG | 0.047 | 0.047 | 0.994 | 2.639 | 1.941 |
| 42 | 16 | 73890482 | 11149814 | 7920 | CACCAAGAAAYCTGTGTAATG | 0.934 | 0.924 | 0.830 | 1.284 | 2.107 |
| 42 | 16 | 73897232 | 17674257 | 7921 | GTGAGTTGGGYCCTGTTTTTT | 0.530 | 0.526 | 0.870 | 1.135 | 2.101 |
| 42 | 16 | 73903741 | 9923574 | 7922 | ATTTTTAAACSTTTTGTGGCT | 0.374 | 0.382 | 1.099 | 0.865 | 2.082 |
| 42 | 16 | 73908476 | 2865532 | 7923 | ATCTTCAGCARAGTCCTAGAT | 1.474 | 1.435 | 0.942 | 0.870 | 1.233 |
| 42 | 16 | 73917597 | 8046109 | 7924 | TTGTGTGCCARATGAGTTTTT | 0.708 | 0.724 | 0.699 | 0.849 | 1.166 |
| 42 | 16 | 73927374 | 8052405 | 7925 | GACTGACTTCMAAATGACAGA | 0.508 | 0.495 | 1.018 | 0.943 | 1.013 |
| 42 | 16 | 73935915 | 9972714 | 7926 | TATTTTATAGYAGGCGTGTAT | 0.010 | 0.009 | 0.489 | 1.015 | 0.990 |
| 42 | 16 | 73945935 | 8050769 | 7927 | CCTCATGATGYATAAAAACAT | 1.194 | 1.151 | 0.670 | 1.103 | 0.998 |
| 42 | 16 | 73955589 | — | 7928 | TCTGCCAGAARGAAAATAAGG | 0.024 | 0.024 | 0.855 | 0.756 | 1.154 |
| 42 | 16 | 73962475 | 8057203 | 7929 | CACATGTTCAYGTGGACTTCA | 1.233 | 1.192 | 1.206 | 0.854 | 1.093 |
| 42 | 16 | 73969226 | 8046184 | 7930 | CCCAAACTGCYTTAGCAATAA | 0.850 | 0.862 | 0.710 | 0.802 | 1.146 |
| 42 | 16 | 73980404 | 10514396 | 7931 | TTAAGTTATAYTGCTTTAGGT | 0.820 | 0.805 | 1.078 | 1.334 | 0.905 |
| 42 | 16 | 74008671 | 7188604 | 7932 | TATATAATAGKTTATTCACTC | 0.074 | 0.075 | 0.724 | 1.069 | 0.944 |
| 42 | 16 | 74033952 | 4888426 | 7933 | ACAGTCTTGGRGTCTTCTGTC | 1.005 | 0.982 | 1.013 | 1.208 | 0.989 |
| 42 | 16 | 74040633 | 12928036 | 7934 | AGGTATGATCRCTATTCTCAA | 0.325 | 0.324 | 0.925 | 0.892 | 1.312 |
| 42 | 16 | 74047910 | 247451 | 7935 | AAAAAAGTCASCCTTAGTCAA | 1.571 | 1.579 | 1.302 | 0.870 | 1.117 |
| 42 | 16 | 74052777 | 11641430 | 7936 | GATGTCTACCYAACCCTCCGT | 0.491 | 0.488 | 0.731 | 0.813 | 1.260 |
| 42 | 16 | 74063566 | 12051137 | 7937 | CTAGGTGAAASTTTTTGAAAT | 0.603 | 0.570 | 0.954 | 1.253 | 1.024 |
| 42 | 16 | 74074404 | — | 7938 | CCAATGGGGCRCCGAAAGAGA | 0.035 | 0.036 | 0.385 | 0.979 | 0.857 |
| 42 | 16 | 74088978 | 2550907 | 7939 | ATATGACTTAYCTATGGCTTC | 0.640 | 0.635 | 0.634 | 1.054 | 0.973 |
| 43 | 16 | 76445659 | 7198177 | 7940 | TTGAAATTCTYGGCACCTGTG | 0.405 | 0.397 | 1.027 | 0.653 | 1.335 |
| 43 | 16 | 76452281 | 12599959 | 7941 | TAGGAAGTTTMAAGCCTGCTT | 0.089 | 0.090 | 0.806 | 1.115 | 1.641 |
| 43 | 16 | 76465091 | 1125690 | 7942 | CATTAAAGCAYAAAGGAAACT | 1.939 | 1.798 | 0.634 | 1.536 | 2.082 |
| 43 | 16 | 76470467 | 3751767 | 7943 | TTTCAAGATTYCTCCAGTTCC | 0.000 | 0.000 | 1.297 | 1.887 | 1.900 |
| 43 | 16 | 76476485 | 12924014 | 7944 | TCGGTGTGGTYACAAGAGCAT | 0.327 | 0.344 | 2.016 | 2.058 | 2.032 |
| 43 | 16 | 76484142 | 382587 | 7945 | CCAGCACAAAYGGTTTCTAGT | 1.834 | 1.960 | 1.535 | 2.308 | 1.872 |
| 43 | 16 | 76491397 | 16946778 | 7946 | AAGCTACACCRTATGCAAGGC | 1.167 | 1.173 | 2.131 | 2.460 | 1.683 |
| 43 | 16 | 76499090 | 17704468 | 7947 | ATGTTCAGTTYTATATCCCCA | 1.164 | 1.248 | 2.441 | 1.834 | 1.838 |
| 43 | 16 | 76508672 | 1429066 | 7948 | TGGAAGCAACRAAAAAGGAAG | 1.021 | 0.990 | 1.412 | 1.851 | 1.714 |
| 43 | 16 | 76517193 | 372627 | 7949 | GAGAAATCTAYCAAACAACAT | 0.610 | 0.608 | 1.336 | 1.722 | 2.607 |
| 43 | 16 | 76523423 | 9937770 | 7950 | CCTGAGCATTKAGTTCATGGG | 0.367 | 0.373 | 0.738 | 1.306 | 1.976 |
| 43 | 16 | 76529180 | 7195268 | 7951 | AGGCGAGTTGRATCCCTTGAA | 0.916 | 1.008 | 0.323 | 0.952 | 2.484 |
| 43 | 16 | 76538629 | 10454707 | 7952 | AAGACCATCTSCTTCTAGTGC | 0.073 | 0.073 | 0.568 | 1.480 | 2.635 |
| 43 | 16 | 76546435 | — | 7953 | CATATTGTGTYCTGTTCATGA | 0.035 | 0.035 | 0.504 | 1.289 | 2.103 |
| 43 | 16 | 76555316 | 16946919 | 7954 | AAGACCTTCARAACAAAACCG | 1.193 | 1.212 | 1.157 | 1.570 | 1.901 |
| 43 | 16 | 76566465 | 373929 | 7955 | TTTTAGGGTTRGCACCTGACA | 0.223 | 0.226 | 1.448 | 1.646 | 2.012 |
| 43 | 16 | 76574323 | 402904 | 7956 | GCCATTTGGGRTATAGGAAAC | 2.522 | 3.060 | 2.149 | 1.480 | 2.170 |
| 43 | 16 | 76581622 | — | 7957 | CAAAAAAATAYAGCCAACATA | 0.591 | 0.568 | 2.057 | 1.585 | 2.071 |
| 43 | 16 | 76587405 | 2252113 | 7958 | CTGACTTGCCYGGACACGTCT | 1.416 | 1.478 | 2.450 | 2.219 | 2.038 |
| 43 | 16 | 76594303 | 8058458 | 7959 | CAATCCTATCKGTGAATTTCT | 0.863 | 0.854 | 1.058 | 2.397 | 2.139 |
| 43 | 16 | 76601272 | 11863422 | 7960 | GCTCTGCGAGYGTTTCCTGTC | 0.545 | 0.541 | 1.638 | 2.261 | 2.955 |
| 43 | 16 | 76607822 | 12597064 | 7961 | GTAAGCCCAARAAGCCCCATA | 0.066 | 0.066 | 1.821 | 1.770 | 3.026 |
| 43 | 16 | 76612446 | 11643023 | 7962 | CCACTGCCCTYCATTTGCACC | 1.697 | 1.622 | 1.542 | 2.085 | 3.342 |
| 43 | 16 | 76634316 | 4888726 | 7963 | ATTGGGGCCAYCCTGTGCATT | 1.726 | 1.784 | 1.530 | 2.924 | 3.503 |
| 43 | 16 | 76647688 | 8045975 | 7964 | TCACTTGCCASCTTGGCCAGG | 0.932 | 0.921 | 2.098 | 2.779 | 3.225 |
| 43 | 16 | 76660674 | 6564507 | 7965 | CTTGTCATTCRATCTTGTCTA | 1.063 | 1.076 | 3.221 | 3.210 | 3.233 |
| 43 | 16 | 76673175 | 16947096 | 7966 | TCAGGAACGAYGATTTATAAC | 1.284 | 1.194 | 3.163 | 3.585 | 3.153 |
| 43 | 16 | 76680185 | — | 7967 | GGGCCTATGCRTAGAGTCAGT | 3.976 | 4.568 | 4.665 | 3.988 | 3.386 |
| 43 | 16 | 76685815 | 1073111 | 7968 | CTCCTTGCTARTAATCAATGT | 0.167 | 0.170 | 3.275 | 3.245 | 3.082 |
| 43 | 16 | 76698607 | 8045088 | 7969 | CCCTTCGAATRCCTCTTGTAG | 2.072 | 2.079 | 3.924 | 2.949 | 3.071 |
| 43 | 16 | 76710658 | 11645006 | 7970 | CACGGTGAACRTTACATGCAA | 0.665 | 0.686 | 0.930 | 2.815 | 2.809 |
| 43 | 16 | 76718209 | 9319518 | 7971 | TTAAACTTGTSTGGCTTTGCA | 0.275 | 0.289 | 0.943 | 2.189 | 2.232 |
| 43 | 16 | 76725829 | 28607706 | 7972 | TTGAGCCCCTYTTCTCTTAGT | 0.284 | 0.287 | 0.432 | 0.597 | 1.841 |
| 43 | 16 | 76738796 | 17650073 | 7973 | GGAACTCTGGSCGTTGATGTA | 0.175 | 0.178 | 0.260 | 0.642 | 1.675 |
| 43 | 16 | 76746301 | 12716853 | 7974 | ATGTTTCCTGRATAATGTGCA | 0.879 | 0.911 | 0.146 | 0.135 | 1.363 |
| 43 | 16 | 76762452 | 7206823 | 7975 | CCTCATTGATWAGCATCGGAA | 0.121 | 0.126 | 0.155 | 0.054 | 0.360 |
| 43 | 16 | 76769919 | 7194833 | 7976 | GAATACATGTSAGTGCTAGAC | 0.022 | 0.023 | 0.123 | 0.088 | 0.334 |
| 43 | 16 | 76775517 | 17573298 | 7977 | CCTTCCACCAYATCAGAAGGT | 0.336 | 0.333 | 0.004 | 0.106 | 0.092 |
| 43 | 16 | 76782865 | 6564516 | 7978 | TATGCCGCCAMAATATATCAA | 0.041 | 0.040 | 0.055 | 0.142 | 0.067 |
| 43 | 16 | 76790899 | 7192037 | 7979 | CTGCGGATCCRGCATCATGAC | 0.020 | 0.020 | 0.143 | 0.037 | 0.056 |
| 43 | 16 | 76797626 | 9938637 | 7980 | AGTTATTGGTRGTGGAATATC | 0.684 | 0.704 | 0.154 | 0.047 | 0.082 |
| 43 | 16 | 76803679 | 2194292 | 7981 | AGGCTGATCARTCTAGGCTGG | 0.429 | 0.429 | 0.171 | 0.082 | 0.111 |
| 43 | 16 | 76811751 | 2067714 | 7982 | TTATTTTTCCRAGCAAGTTTT | 0.370 | 0.374 | 0.231 | 0.062 | 0.041 |
| 43 | 16 | 76822238 | 12931696 | 7983 | CACACCAATGRTATATATTTT | 0.032 | 0.032 | 0.124 | 0.152 | 0.125 |
| 43 | 16 | 76827833 | 7204887 | 7984 | TGACAGACACMTTAAGAGCTT | 0.253 | 0.260 | 0.081 | 0.258 | 0.304 |
| 43 | 16 | 76830780 | 11649150 | 7985 | TCATTCATGCYGAGACCCTGA | 0.404 | 0.388 | 0.136 | 0.118 | 0.250 |
| 43 | 16 | 76868733 | 4380066 | 7986 | AAAATGATGTMAGGATCCTTT | 0.241 | 0.242 | 0.291 | 0.262 | 0.387 |
| 43 | 16 | 76876919 | 4887950 | 7987 | ATGAAGCCCCRTAGGATGGCT | 0.621 | 0.625 | 0.236 | 0.518 | 0.496 |
| 43 | 16 | 76894438 | 8051705 | 7988 | ATAGATGCTCYTATTGGAACA | 0.515 | 0.518 | 0.546 | 0.520 | 0.403 |
| 43 | 16 | 76904717 | 4887952 | 7989 | TAGAACGTCTRAACAGTGCTG | 0.062 | 0.064 | 1.024 | 0.687 | 0.590 |
| 43 | 16 | 76920881 | 8048594 | 7990 | CTTGAGGCTTYGTGGCAAATG | 1.143 | 1.130 | 0.705 | 0.717 | 0.580 |
| 43 | 16 | 76933108 | 7498411 | 7991 | AGCATCGAGTRTGGAAACCTC | 1.213 | 1.178 | 0.809 | 0.701 | 1.001 |
| 43 | 16 | 76945965 | 4587988 | 7992 | ATATGCTTAGRAATATCCAGC | 0.029 | 0.029 | 1.038 | 0.927 | 1.123 |
| 43 | 16 | 76959950 | 4459554 | 7993 | ATATCCTCAAMCCAAGGAGAA | 0.772 | 0.763 | 0.591 | 0.855 | 1.104 |
| 43 | 16 | 76971907 | 8058540 | 7994 | CCATTCAGCCSTATCAGTGAA | 0.467 | 0.448 | 0.632 | 1.409 | 1.083 |
| 43 | 16 | 76984378 | 8058511 | 7995 | CAGCGGTCGTYGGCCAGTGTG | 0.286 | 0.291 | 0.767 | 1.238 | 0.966 |
| 43 | 16 | 76997097 | 8051395 | 7996 | CAGGAAATATYCTCTTTTAGG | 1.263 | 1.292 | 1.174 | 0.948 | 0.956 |
| 43 | 16 | 77011368 | 6564541 | 7997 | ATGAGCATAARATCTAAATAG | 0.000 | 0.000 | 1.303 | 0.996 | 0.987 |
| 43 | 16 | 77024259 | 3764342 | 7998 | TCTCAGTAATMACATTGTCCA | 1.555 | 1.575 | 1.386 | 0.845 | 0.766 |
| 43 | 16 | 77031763 | 4297688 | 7999 | CTAGAATACTSCTCCTCCACT | 0.974 | 0.972 | 0.908 | 0.877 | 0.628 |
| 43 | 16 | 77050888 | 2738646 | 8000 | CATGCGGTATSTGCCATTTTG | 0.240 | 0.236 | 0.807 | 0.823 | 0.634 |
| 43 | 16 | 77056971 | 2738664 | 8001 | TTTCATTAGTRACTCACATGG | 0.380 | 0.383 | 0.419 | 0.524 | 0.831 |
| 43 | 16 | 77063859 | 16947630 | 8002 | TTCTCTTGCTKTTCTCTAAAG | 0.184 | 0.176 | 0.115 | 0.592 | 0.791 |
| 43 | 16 | 77070054 | 2667556 | 8003 | CGTTGAGTTASTGGTTCACTG | 0.000 | 0.000 | 0.109 | 0.212 | 0.893 |
| 43 | 16 | 77076412 | 2738696 | 8004 | TTATAGATGARGTTCCCTTAG | 0.113 | 0.115 | 0.151 | 0.413 | 0.665 |
| 43 | 16 | 77082641 | 12444091 | 8005 | CCCTCTCAGTYAGCTGCTACA | 0.211 | 0.231 | 0.113 | 0.397 | 0.769 |
| 43 | 16 | 77088538 | 2667590 | 8006 | TTAGGTGTATSTCTGCTCATG | 0.561 | 0.569 | 0.596 | 0.510 | 0.599 |
| 43 | 16 | 77095567 | 13331582 | 8007 | TAATAAATAASTGAAATACCA | 0.106 | 0.104 | 0.652 | 0.492 | 0.870 |
| 43 | 16 | 77102849 | 1465099 | 8008 | CGTTGCAGGCRCGTTGAATGT | 1.715 | 1.653 | 0.926 | 0.593 | 1.110 |
| 43 | 16 | 77109437 | 11641340 | 8009 | TATGTCTTTGRGCTTGGTAAT | 0.266 | 0.260 | 0.725 | 0.856 | 1.201 |
| 43 | 16 | 77115457 | 16947928 | 8010 | GGTGATTCCARTGTGAGTGGT | 0.811 | 0.780 | 1.108 | 1.703 | 1.520 |
| 43 | 16 | 77121333 | 11645676 | 8011 | TGTTGGTTTCYGTGTTGATGA | 0.177 | 0.169 | 0.577 | 1.893 | 1.742 |
| 43 | 16 | 77127745 | 16948025 | 8012 | AAGGGTGTGAYCAGTTCCATG | 0.779 | 0.718 | 1.591 | 2.106 | 2.399 |
| 43 | 16 | 77135652 | 16948047 | 8013 | GTAAAACAGTRGATGTCTTTC | 0.751 | 0.736 | 1.613 | 1.804 | 2.454 |
| 43 | 16 | 77145092 | 2667505 | 8014 | TGATAGAGAASCTTTCCCTAG | 1.949 | 1.979 | 1.867 | 2.366 | 2.073 |
| 43 | 16 | 77154108 | 2859627 | 8015 | ACAATCTGATYGCTCCCCTGA | 1.011 | 0.979 | 2.064 | 2.628 | 2.599 |
| 43 | 16 | 77156356 | 9928690 | 8016 | TTTTAACAAGRCAAGTAGAAA | 0.731 | 0.730 | 2.423 | 2.560 | 2.912 |
| 43 | 16 | 77171105 | 16948109 | 8017 | AGAACTATGTKTGTTTTCACT | 1.115 | 1.104 | 1.902 | 2.016 | 2.977 |
| 43 | 16 | 77180388 | 11862167 | 8018 | TTGCTGGGAAYGTGGCCATCA | 1.201 | 1.210 | 1.587 | 2.189 | 2.720 |
| 43 | 16 | 77188320 | 17640520 | 8019 | ACTCTCTTGAYGCTATCCTCA | 1.391 | 1.384 | 1.157 | 1.968 | 2.653 |
| 43 | 16 | 77206499 | 12599563 | 8020 | AAATTCTATGKGTAAAATAAT | 0.071 | 0.071 | 1.117 | 1.805 | 2.184 |
| 43 | 16 | 77213355 | 2738566 | 8021 | TTGAAATTGTMCTGGTTTTTC | 0.160 | 0.158 | 1.448 | 1.775 | 2.108 |
| 43 | 16 | 77219165 | 2550608 | 8022 | GAAAAGTTGASTAGACAAAAG | 1.018 | 0.984 | 0.882 | 1.479 | 1.667 |
| 43 | 16 | 77230099 | 2550620 | 8023 | TCAGATTCCAMTCGAATACAT | 1.920 | 1.920 | 1.254 | 1.001 | 1.628 |
| 43 | 16 | 77238229 | 2548841 | 8024 | TTCCTTTTATYTCCCATCAGC | 0.274 | 0.260 | 1.394 | 0.712 | 1.444 |
| 43 | 16 | 77245970 | — | 8025 | GATTCTGATTKCTCCGTGATA | 0.478 | 0.485 | 0.866 | 1.063 | 1.192 |
| 43 | 16 | 77256370 | 17795127 | 8026 | AATGAACTTGKGAAGGTTTGC | 0.541 | 0.536 | 0.278 | 1.279 | 0.846 |
| 43 | 16 | 77262902 | 17722185 | 8027 | CCAGTATAGAYAACTTGTGAT | 0.017 | 0.017 | 0.486 | 0.909 | 0.534 |
| 43 | 16 | 77268743 | 6564575 | 8028 | ATGTTGAGATYACATACATCC | 0.656 | 0.654 | 0.509 | 0.317 | 0.527 |
| 43 | 16 | 77276578 | 2738630 | 8029 | AGGACTGGGASAGATCTGTGA | 0.769 | 0.785 | 0.393 | 0.283 | 0.598 |
| 43 | 16 | 77284472 | 7195153 | 8030 | CTTGCCAGCARCCTTTTAGAT | 0.564 | 0.527 | 0.502 | 0.240 | 0.763 |
| 43 | 16 | 77289752 | — | 8031 | TTTTTGGGGTWTCTTCCAGCC | 0.253 | 0.266 | 0.278 | 0.137 | 0.476 |
| 43 | 16 | 77310766 | 2042433 | 8032 | TAAATCTTCTRTCAGGTGATG | 0.281 | 0.282 | 0.146 | 0.218 | 0.566 |
| 43 | 16 | 77322980 | 8052934 | 8033 | TTTATTTGGGYTTTTTCGGTT | 0.112 | 0.114 | 0.045 | 0.485 | 0.499 |
| 43 | 16 | 77330775 | 16948419 | 8034 | CAACAATTTTKGAGGCACCCA | 0.291 | 0.297 | 0.062 | 0.501 | 0.424 |
| 43 | 16 | 77337515 | — | 8035 | GTGATTTGTCSAAGGACATCG | 0.046 | 0.044 | 0.499 | 0.529 | 0.460 |
| 43 | 16 | 77344287 | 12599398 | 8036 | TCCTGGTCCCRTCTTGGCTGG | 0.396 | 0.412 | 0.860 | 0.515 | 0.334 |
| 43 | 16 | 77353386 | 10514443 | 8037 | GTGCAGTGAGRATGAATCAAC | 1.645 | 1.755 | 1.113 | 0.482 | 0.270 |
| 43 | 16 | 77360990 | 7201295 | 8038 | TTTTTACCATRTCTGTTACTC | 0.819 | 0.829 | 1.210 | 0.507 | 0.261 |
| 43 | 16 | 77368476 | 4887985 | 8039 | GAGGAAAATTRGGAATGAAGT | 0.621 | 0.631 | 0.994 | 0.429 | 0.351 |
| 43 | 16 | 77375785 | 7191931 | 8040 | AATCAGAAATYTGTGACTCCA | 0.228 | 0.231 | 0.329 | 0.577 | 0.406 |
| 43 | 16 | 77382825 | 2550595 | 8041 | CTCCFGAAACRTAATTTGGAG | 0.175 | 0.177 | 0.091 | 0.615 | 0.407 |
| 43 | 16 | 77401769 | 1124597 | 8042 | ATGTGGACACRTGCAGCCACA | 0.189 | 0.191 | 0.032 | 0.309 | 0.597 |
| 43 | 16 | 77439717 | 7501409 | 8043 | TTCAAGTCATRTTTGCCCCCA | 0.039 | 0.040 | 0.077 | 0.233 | 0.826 |
| 43 | 16 | 77453496 | 8063104 | 8044 | GGGTGTGTCTRTATCTATACA | 0.000 | 0.000 | 0.249 | 0.118 | 0.833 |
| 43 | 16 | 77461185 | 16948659 | 8045 | CTTAGCAGAGYCAAAAGCCAG | 0.518 | 0.486 | 0.414 | 0.282 | 0.476 |
| 43 | 16 | 77470888 | 2656620 | 8046 | CCCTGGTCTTMAAGTCCACTC | 0.732 | 0.745 | 0.448 | 0.489 | 0.366 |
| 43 | 16 | 77478402 | 4887990 | 8047 | TTGAGTTTTCRTATTGTCAGT | 0.534 | 0.559 | 0.669 | 0.595 | 0.293 |
| 43 | 16 | 77484117 | 11647676 | 8048 | ATTGTGATACWATAGGAGCCG | 0.087 | 0.091 | 0.810 | 0.741 | 0.267 |
| 43 | 16 | 77491900 | 8064053 | 8049 | TCAAGAAACCSACAGAGGTCC | 0.969 | 0.960 | 0.678 | 0.691 | 0.254 |
| 43 | 16 | 77497905 | 16948804 | 8050 | CACTTGTGCTSTGCAAAATCA | 0.799 | 0.818 | 0.632 | 0.615 | 0.236 |
| 43 | 16 | 77503965 | 8060900 | 8051 | AACCAGCAGGRTTATATCCCC | 0.494 | 0.482 | 0.768 | 0.409 | 0.234 |
| 43 | 16 | 77511358 | 7192071 | 8052 | TACCAAACACRTAAGCTTATT | 0.461 | 0.456 | 0.443 | 0.278 | 0.288 |
| 43 | 16 | 77528453 | 16948943 | 8053 | ATTAAGTGGARATATAGGTTT | 0.358 | 0.355 | 0.184 | 0.260 | 0.487 |
| 43 | 16 | 77535072 | 13331318 | 8054 | AGTGTAGGACRCTGGAAAAGT | 0.287 | 0.277 | 0.093 | 0.096 | 0.370 |
| 43 | 16 | 77541656 | 1995548 | 8055 | GCTCAAATGCRTGACATGATG | 0.153 | 0.152 | 0.028 | 0.086 | 0.442 |
| 43 | 16 | 77549079 | 9319530 | 8056 | AAGGGATTTTSAAGCAGTTGT | 0.112 | 0.106 | 0.008 | 0.270 | 0.432 |
| 43 | 16 | 77557050 | 8052893 | 8057 | TAGCATCTCTRGCATAAACTA | 0.004 | 0.004 | 0.050 | 0.212 | 0.814 |
| 43 | 16 | 77563991 | 4888896 | 8058 | GTTCCATTTAMATTGAATGGC | 0.089 | 0.086 | 0.443 | 0.331 | 1.032 |
| 43 | 16 | 77570962 | 8063569 | 8059 | GCTCCTGTGASGGTTTCTACA | 0.712 | 0.734 | 0.482 | 0.270 | 0.897 |
| 43 | 16 | 77576754 | 16949152 | 8060 | GTTGTCTCCARTGTGCCTTGT | 1.472 | 1.505 | 0.909 | 1.028 | 0.849 |
| 43 | 16 | 77583096 | 10492907 | 8061 | CTTTCCTATAYAACGCAAGCC | 0.206 | 0.201 | 0.908 | 1.856 | 0.861 |
| 43 | 16 | 77593245 | 10492905 | 8062 | CTGCAGTGGTRTCGCTTTAGA | 0.850 | 0.835 | 1.943 | 1.790 | 0.967 |
| 43 | 16 | 77601455 | 1469135 | 8063 | AAGCTGCTATSCTTGCAAAGG | 0.019 | 0.020 | 1.800 | 1.888 | 1.053 |
| 43 | 16 | 77609347 | 12932339 | 8064 | AAAAGCAGGCRAAATAGAATA | 2.388 | 2.454 | 1.643 | 1.743 | 1.421 |
| 43 | 16 | 77614961 | 16949262 | 8065 | ACCATCCACCRCAGGCAAACA | 1.561 | 1.536 | 1.436 | 1.248 | 1.501 |
| 43 | 16 | 77621304 | 2656645 | 8066 | CATAAACGCAYATGGTCACTT | 0.160 | 0.156 | 1.623 | 1.336 | 1.433 |
| 43 | 16 | 77627954 | 16949326 | 8067 | CCTGAATTATYGGAGCTCTTG | 0.234 | 0.230 | 0.713 | 1.460 | 1.203 |
| 43 | 16 | 77634311 | 2194344 | 8068 | TAAGTGATGCRTCAGAATTTC | 0.371 | 0.391 | 0.213 | 1.512 | 0.967 |
| 43 | 16 | 77641440 | 16949415 | 8069 | GGAATGGGAGSAGTTTAAACA | 0.605 | 0.604 | 0.508 | 0.509 | 1.009 |
| 43 | 16 | 77648924 | 9928080 | 8070 | GAGCTGAGCCRCACTTGGAGA | 0.406 | 0.418 | 0.477 | 0.115 | 0.942 |
| 43 | 16 | 77661543 | 17726900 | 8071 | AGCTATTTCCSTAATACCAAG | 0.883 | 0.847 | 0.306 | 0.298 | 1.112 |
| 43 | 16 | 77667343 | 11150133 | 8072 | TCTGCTCTAAYGTTGGATCAT | 0.170 | 0.168 | 0.141 | 0.349 | 0.472 |
| 43 | 16 | 77674005 | 6564644 | 8073 | CCAGAAAGTCSCGAGTGTTTT | 0.061 | 0.041 | 0.326 | 0.366 | 0.336 |
| 43 | 16 | 77681077 | 17727687 | 8074 | CTTCGAATTGKTGCACTAGAG | 0.112 | 0.108 | 0.175 | 0.331 | 0.918 |
| 43 | 16 | 77687044 | 8052725 | 8075 | AGTAGGCTCTMACCTGGCAGG | 0.920 | 0.915 | 0.256 | 0.293 | 0.977 |
| 43 | 16 | 77690771 | 17727813 | 8076 | GTAAGGTCCCRTGACAATTAG | 0.415 | 0.408 | 0.447 | 0.325 | 0.873 |
| 43 | 16 | 77706366 | 7185485 | 8077 | AGTTAGACAAYTAGCTCGCCC | 0.475 | 0.467 | 0.504 | 1.078 | 0.690 |
| 43 | 16 | 77713723 | 2005036 | 8078 | GTTCAGGCGGYATTAACCCCC | 0.510 | 0.486 | 0.548 | 1.260 | 0.770 |
| 43 | 16 | 77720121 | 12923469 | 8079 | ATTTGCCAAGKTTTGAAAAGT | 0.243 | 0.251 | 1.521 | 1.252 | 0.644 |
| 43 | 16 | 77726730 | 4611476 | 8080 | TCCTTTTCCCRTAATTTTATG | 0.985 | 1.004 | 1.511 | 0.853 | 0.703 |
| 43 | 16 | 77729566 | 1424113 | 8081 | GTCCTTGATCRTTTTTCTTGG | 2.263 | 2.155 | 1.238 | 0.965 | 0.798 |
| 43 | 16 | 77742894 | 7201082 | 8082 | GTAACCAATAYCGCTCCTTCT | 0.391 | 0.396 | 1.038 | 0.692 | 0.793 |
| 43 | 16 | 77751639 | 6564651 | 8083 | TTTTGATGTAYAGACATCCCT | 0.108 | 0.107 | 0.926 | 0.842 | 0.914 |
| 43 | 16 | 77757915 | 11150140 | 8084 | CATACGGGCARTAGTCCGCAT | 0.084 | 0.085 | 0.311 | 0.872 | 1.064 |
| 43 | 16 | 77764739 | 6564653 | 8085 | TGTATGTTTGSGGTACAACTC | 0.811 | 0.795 | 0.284 | 0.628 | 0.929 |
| 43 | 16 | 77770740 | 12716871 | 8086 | GAGAGGTACTSTTAGCAAATC | 0.374 | 0.387 | 0.317 | 0.396 | 1.054 |
| 43 | 16 | 77777161 | 17730134 | 8087 | AGAAATGAGARTGAGGATAAA | 0.336 | 0.343 | 0.332 | 0.575 | 0.986 |
| 43 | 16 | 77783493 | 386497 | 8088 | CATAGGGTATYTCCAGATAAT | 0.321 | 0.313 | 0.527 | 0.565 | 0.846 |
| 43 | 16 | 77789947 | 383673 | 8089 | GGAGCAACCTMACTAGAGACA | 0.163 | 0.166 | 0.837 | 0.835 | 0.411 |
| 43 | 16 | 77797274 | 449842 | 8090 | CGCACCTACAYGCGTGGGAAC | 1.332 | 1.396 | 0.710 | 0.619 | 0.596 |
| 43 | 16 | 77804563 | 395050 | 8091 | GCAAGACGGARAAAATGCTTC | 1.114 | 1.153 | 0.990 | 0.644 | 0.599 |
| 43 | 16 | 77810494 | 270425 | 8092 | TGCCCTGCTGMATCTTAGGCG | 0.055 | 0.057 | 0.992 | 0.576 | 1.027 |
| 43 | 16 | 77829050 | 9924583 | 8093 | TTCTTAAACTMTGATCTATCC | 1.096 | 1.105 | 0.606 | 0.897 | 0.952 |
| 43 | 16 | 77841837 | 16950049 | 8094 | TTTTTGGCACKTGGCTCTTTA | 0.001 | 0.001 | 0.173 | 0.917 | 1.094 |
| 43 | 16 | 77849307 | 437620 | 8095 | ATTTTGCCCARATGTTATCAG | 0.470 | 0.483 | 0.745 | 1.195 | 1.162 |
| 43 | 16 | 77856543 | 270433 | 8096 | TGCATAAATGRAAGTCATTAT | 0.056 | 0.054 | 0.291 | 0.903 | 1.285 |
| 43 | 16 | 77863078 | 17796919 | 8097 | TCCATCTCTAYTGCTAAGGAA | 1.399 | 1.393 | 1.302 | 1.262 | 1.563 |
| 43 | 16 | 77869153 | 4888944 | 8098 | AATTTGAGACMACTTTTCTGT | 0.102 | 0.096 | 1.230 | 1.066 | 1.195 |
| 43 | 16 | 77875406 | — | 8099 | TAGTAGTGATKGATGGCAGAA | 1.910 | 1.878 | 1.862 | 1.400 | 1.094 |
| 43 | 16 | 77881728 | 17724291 | 8100 | TTTCCCATAARTCATACGGGC | 0.411 | 0.401 | 1.278 | 1.488 | 1.226 |
| 43 | 16 | 77895943 | 9924169 | 8101 | AGAACTAGCCYTTTTGATTCC | 0.694 | 0.856 | 1.571 | 1.480 | 0.981 |
| 43 | 16 | 77902014 | 12600246 | 8102 | CCGCCTCCGCSACATTTTGCA | 0.628 | 0.638 | 0.704 | 1.178 | 1.189 |
| 43 | 16 | 77908301 | 12596374 | 8103 | TGGACACCAAMAAAGTAGGAG | 0.617 | 0.543 | 0.642 | 1.308 | 1.016 |
| 43 | 16 | 77914236 | 16944185 | 8104 | CCTGCCAAAGRGTTCTAGTTT | 0.482 | 0.484 | 0.633 | 0.665 | 1.033 |
| 43 | 16 | 77921315 | 7191820 | 8105 | CATACAGCATRAAGAACAGAC | 0.336 | 0.308 | 0.497 | 0.740 | 0.725 |
| 43 | 16 | 77927430 | 6564662 | 8106 | CACAGGGCTGSGCTCAGGGCA | 0.799 | 0.808 | 0.477 | 0.471 | 0.750 |
| 43 | 16 | 77933390 | 16950304 | 8107 | TATGAACAACRACAAAATTTA | 0.321 | 0.341 | 0.531 | 0.321 | 0.689 |
| 43 | 16 | 77939410 | 7198888 | 8108 | GTCCTTTCAAKGATGTCTCTA | 0.511 | 0.492 | 0.422 | 0.293 | 0.687 |
| 43 | 16 | 77946548 | 9936888 | 8109 | CTATCTTGCASGATCCTAGTG | 0.633 | 0.629 | 0.183 | 0.231 | 0.493 |
| 43 | 16 | 77960029 | 4243168 | 8110 | ACCAGTAGGASCCCTGGTGCT | 0.033 | 0.034 | 0.206 | 0.601 | 0.493 |
| 43 | 16 | 77970762 | 12325433 | 8111 | TGACAGTCTASTTTGCAGTTG | 0.110 | 0.111 | 0.118 | 0.474 | 0.381 |
| 43 | 16 | 77999427 | 4530155 | 8112 | AGAGGCGGGCSCCATAAGAGA | 0.412 | 0.429 | 0.475 | 0.388 | 0.423 |
| 43 | 16 | 78006279 | 16950383 | 8113 | TTCATGTGTTKTCTTAGCATT | 0.175 | 0.182 | 0.636 | 0.432 | 0.550 |
| 43 | 16 | 78012972 | 8051128 | 8114 | CGGAAGGGAARATGATAGACA | 1.680 | 1.645 | 0.620 | 0.277 | 0.403 |
| 43 | 16 | 78023063 | 4888977 | 8115 | GAATGCTAACYTGTGTTTTCC | 0.395 | 0.392 | 0.733 | 0.469 | 0.336 |
| 43 | 16 | 78030355 | 7190849 | 8116 | GAGACATCCTMAGATTAACAA | 0.098 | 0.096 | 0.668 | 0.710 | 0.317 |
| 43 | 16 | 78037195 | 11150162 | 8117 | TTTATTATTAYAATTGCTCCT | 0.617 | 0.634 | 0.247 | 0.630 | 0.219 |
| 43 | 16 | 78049764 | 1862720 | 8118 | CTATTACCAAMAGAAGACTAC | 0.113 | 0.109 | 0.420 | 0.575 | 0.214 |
| 44 | 18 | 22503963 | 17607947 | 8119 | GGATGAATTCRGTTCTCTGTG | 0.195 | 0.193 | 0.223 | 0.085 | 0.019 |
| 44 | 18 | 22517853 | 17608457 | 8120 | TATCTGATGCKAATGGTATAT | 0.886 | 0.862 | 0.201 | 0.042 | 0.021 |
| 44 | 18 | 22524486 | — | 8121 | GATATGTCACWGAGTAGGATA | 0.561 | 0.579 | 0.256 | 0.030 | 0.037 |
| 44 | 18 | 22530423 | 16942566 | 8122 | GCAGAGTTATRGGAGACTTCT | 0.097 | 0.091 | 0.198 | 0.028 | 0.025 |
| 44 | 18 | 22535819 | 16960655 | 8123 | ATATTTTTTAYGGGAAAAAGC | 0.117 | 0.119 | 0.028 | 0.051 | 0.016 |
| 44 | 18 | 22543855 | 470306 | 8124 | CCTTCTCCAAYGGCTTAATAA | 0.018 | 0.018 | 0.001 | 0.091 | 0.011 |
| 44 | 18 | 22549985 | 470677 | 8125 | CACTTCTCTCKTGTTGGAGCT | 0.067 | 0.067 | 0.005 | 0.031 | 0.015 |
| 44 | 18 | 22555942 | 470455 | 8126 | TGTTTATGCTRATGAAGTGAC | 0.094 | 0.091 | 0.035 | 0.010 | 0.018 |
| 44 | 18 | 22563606 | 7234618 | 8127 | TGGGTAGAATYGAGAGAAAGA | 0.160 | 0.312 | 0.076 | 0.009 | 0.020 |
| 44 | 18 | 22569929 | 4800265 | 8128 | AGGTTTTCACRTGATGCTGTG | 0.479 | 0.474 | 0.111 | 0.013 | 0.091 |
| 44 | 18 | 22579380 | — | 8129 | ACAAGGAAGCRGGCACTGGGC | 0.314 | 0.310 | 0.101 | 0.013 | 0.071 |
| 44 | 18 | 22595324 | — | 8130 | TGTGAGCAGCSCTCAGTAAAG | 0.195 | 0.210 | 0.089 | 0.021 | 0.095 |
| 44 | 18 | 22608012 | 1562701 | 8131 | CTCCCTTGCTRTAATAAGTGC | 0.070 | 0.070 | 0.026 | 0.313 | 0.185 |
| 44 | 18 | 22613927 | 573998 | 8132 | CCTAACACGTMATTTGGTGTT | 0.281 | 0.249 | 0.014 | 0.333 | 0.516 |
| 44 | 18 | 22621971 | 487455 | 8133 | TCTTGAAAAGYGGTGAAAATG | 0.040 | 0.039 | 0.477 | 0.303 | 0.596 |
| 44 | 18 | 22626379 | 621765 | 8134 | AGCCCCAAACRTTAACTTCTT | 0.173 | 0.171 | 0.620 | 0.483 | 0.865 |
| 44 | 18 | 22640533 | 16942713 | 8135 | GCCTGAAGAAKCCTGAGAATG | 2.015 | 2.137 | 0.669 | 1.042 | 0.766 |
| 44 | 18 | 22647211 | 16942751 | 8136 | GCTGCCTAACMAACATTCCCT | 0.391 | 0.383 | 1.170 | 1.180 | 1.204 |
| 44 | 18 | 22654052 | 17690930 | 8137 | TTATTTTACCRTCCTGAAAGT | 0.343 | 0.342 | 2.014 | 1.487 | 1.131 |
| 44 | 18 | 22660468 | 11659932 | 8138 | TGAGAGTGGGYGGACATGTAG | 0.924 | 0.948 | 1.160 | 1.490 | 1.155 |
| 44 | 18 | 22667416 | 16942801 | 8139 | GTGCAGACCGWTAAAGACACA | 1.717 | 1.688 | 1.571 | 2.367 | 1.487 |
| 44 | 18 | 22673906 | 16942835 | 8140 | TTTGGGGAGAWCACATTCCAG | 0.383 | 0.389 | 1.377 | 1.399 | 1.479 |
| 44 | 18 | 22684849 | 12455617 | 8141 | CTTAATGTTGMCTTAAACCCA | 0.937 | 0.921 | 1.872 | 1.379 | 1.708 |
| 44 | 18 | 22692538 | 11661256 | 8142 | TGAGCCTTTAWCTTCTCTATG | 0.091 | 0.094 | 0.836 | 1.619 | 1.836 |
| 44 | 18 | 22703018 | 11662318 | 8143 | CTGTGCCCCAYGCCCCACAGA | 1.734 | 1.632 | 0.781 | 1.290 | 2.118 |
| 44 | 18 | 22710424 | 589598 | 8144 | TATGTTGACAYCATACTAGGA | 0.105 | 0.108 | 0.844 | 0.898 | 2.090 |
| 44 | 18 | 22715430 | 3974646 | 8145 | TGATGAGGAAKGACACTGTTT | 0.255 | 0.262 | 0.870 | 1.011 | 1.894 |
| 44 | 18 | 22733560 | 630285 | 8146 | CCAGGTTTGCRTGCCTACCTA | 1.106 | 1.075 | 0.518 | 1.584 | 1.681 |
| 44 | 18 | 22743410 | 162632 | 8147 | TTAATTCTGAYACAGGTCGTC | 0.174 | 0.165 | 0.770 | 1.587 | 1.370 |
| 44 | 18 | 22749587 | 1143734 | 8148 | AAGAAGGTTGSTTTTGTTGCT | 0.885 | 0.851 | 1.752 | 1.077 | 1.455 |
| 44 | 18 | 22756491 | 16942955 | 8149 | AAAGACATGTRACTACAGATT | 0.797 | 0.749 | 1.414 | 1.125 | 1.388 |
| 44 | 18 | 22763991 | 163221 | 8150 | CTGGATTAAGYATCTACTGGT | 2.722 | 2.809 | 1.313 | 1.324 | 1.350 |
| 44 | 18 | 22770692 | 11663286 | 8151 | GCAGAGAAGGWTATGATTTGA | 0.221 | 0.218 | 1.172 | 1.224 | 0.945 |
| 44 | 18 | 22777715 | 163048 | 8152 | AAACAGTTCTRTAGAAGGACA | 0.130 | 0.127 | 1.213 | 1.361 | 1.048 |
| 44 | 18 | 22784157 | 16943030 | 8153 | AACTGCTGTGYCCTGGGCCTA | 0.393 | 0.399 | 0.448 | 1.217 | 1.009 |
| 44 | 18 | 22790787 | 444673 | 8154 | ATGTTTGAAAWCTCTGGTTGG | 1.104 | 1.042 | 0.576 | 0.960 | 0.807 |
| 44 | 18 | 22798300 | 1154215 | 8155 | ATTCTCAACCKCCCACCTGTT | 0.563 | 0.579 | 0.687 | 0.324 | 0.834 |
| 44 | 18 | 22803905 | 1657316 | 8156 | GTTATCATTAYATTTCTTGGT | 0.524 | 0.513 | 0.496 | 0.308 | 0.729 |
| 44 | 18 | 22804050 | 4093026 | 8157 | GGTTGGGTGCYTAAAATTGCC | 0.337 | 0.324 | 0.270 | 0.314 | 0.591 |
| 44 | 18 | 22811160 | 1785548 | 8158 | TAATTTGTACYGATGCTTAAA | 0.005 | 0.006 | 0.131 | 0.300 | 0.193 |
| 44 | 18 | 22826887 | 16943138 | 8159 | GTATTCATTTRCAGGCTCTTT | 0.535 | 0.538 | 0.072 | 0.137 | 0.225 |
| 44 | 18 | 22833854 | 1154208 | 8160 | GGTACTTAGAYGTTAAGTGGG | 0.108 | 0.096 | 0.082 | 0.073 | 1.465 |
| 44 | 18 | 22841488 | 16960687 | 8161 | AGAGCAGATAYAATCACTGTA | 0.255 | 0.239 | 0.154 | 0.083 | 1.311 |
| 44 | 18 | 22851934 | 1436906 | 8162 | GTTTCCTTAGMTTAAGCCTCA | 0.359 | 0.360 | 0.073 | 0.099 | 1.042 |
| 44 | 18 | 22867639 | 6508464 | 8163 | CTTTTCTGGGYAATCTCATTC | 0.345 | 0.322 | 0.186 | 1.844 | 0.917 |
| 44 | 18 | 22874298 | 12604227 | 8164 | ATCACGTCTARGAAAATTTAA | 0.173 | 0.169 | 0.252 | 1.449 | 1.277 |
| 44 | 18 | 22881674 | 7505854 | 8165 | TATCACAGGARTAGTGGGTTG | 0.588 | 0.588 | 2.626 | 1.426 | 1.364 |
| 44 | 18 | 22892113 | 1485807 | 8166 | AAATAGGAATRCTAGTCTTAA | 0.453 | 0.440 | 1.938 | 1.447 | 1.353 |
| 44 | 18 | 22898135 | 6508467 | 8167 | AAACCCTGGGMAAGTGATAAT | 4.907 | 4.422 | 1.977 | 1.870 | 1.211 |
| 44 | 18 | 22905657 | 9957079 | 8168 | CATAAATACAKTGTTGAGAAA | 0.098 | 0.095 | 2.126 | 1.886 | 1.292 |
| 44 | 18 | 22921346 | 12954928 | 8169 | ACTAGCAAAAYCACCTTTCTA | 0.062 | 0.064 | 2.779 | 1.776 | 1.518 |
| 44 | 18 | 22928009 | 8088410 | 8170 | TATCTGCAAARGGGTTATTTT | 0.099 | 0.099 | 0.520 | 1.937 | 1.463 |
| 44 | 18 | 22943704 | 919140 | 8171 | GGGGCTCCTGSTGAATTATAA | 1.810 | 1.771 | 0.523 | 1.938 | 1.515 |
| 44 | 18 | 22952382 | — | 8172 | TTACTTCTCCYTACATTATGT | 0.000 | 0.000 | 0.494 | 0.455 | 1.874 |
| 44 | 18 | 22959715 | 12969203 | 8173 | CCTGAGAAGAYAGCCGTCAAT | 0.065 | 0.066 | 0.694 | 0.454 | 2.057 |
| 44 | 18 | 22966551 | 9964571 | 8174 | GAACAAGGCARTACCTTGGGG | 0.091 | 0.112 | 0.364 | 0.670 | 2.011 |
| 44 | 18 | 22972845 | 4800790 | 8175 | GTAACAACTAYGAAAAAAAAA | 0.492 | 0.484 | 0.230 | 1.285 | 0.533 |
| 44 | 18 | 22981800 | 4800792 | 8176 | ATTCTGTTCTRTTGATTCAAT | 1.154 | 1.118 | 0.465 | 0.725 | 0.775 |
| 44 | 18 | 22991320 | 8096346 | 8177 | TGAAATTAATRTGATTTGTTT | 0.061 | 0.095 | 1.286 | 0.595 | 1.009 |
| 44 | 18 | 23005211 | 9304504 | 8178 | CAATCAAAAARTGGCTTATGA | 0.708 | 0.702 | 1.266 | 0.633 | 1.008 |
| 44 | 18 | 23011541 | 2195032 | 8179 | CTTATTTGGTKTTCCCATTTT | 1.599 | 1.521 | 0.678 | 0.941 | 0.712 |
| 44 | 18 | 23016400 | 8088724 | 8180 | TTCAAGTACAYATCAATTATT | 0.434 | 0.402 | 0.729 | 1.073 | 0.587 |
| 44 | 18 | 23027139 | 4438389 | 8181 | ATTAAATACARCAAAATGGCA | 0.187 | 0.184 | 0.889 | 0.685 | 0.703 |
| 44 | 18 | 23036910 | — | 8182 | TCACTGGTAARCCATGGCTGC | 0.152 | 0.146 | 0.545 | 0.916 | 0.888 |
| 44 | 18 | 23039432 | — | 8183 | ACAGAGACTGRTAAGTATTTC | 0.989 | 0.974 | 0.396 | 0.707 | 0.847 |
| 44 | 18 | 23060734 | 17712675 | 8184 | AAAGATCTTASGCAAACAGGA | 0.832 | 0.866 | 0.615 | 0.338 | 0.766 |
| 44 | 18 | 23070175 | 16943382 | 8185 | GCAGAAAAGASACTGTCTAGG | 0.086 | 0.083 | 0.574 | 0.485 | 0.790 |
| 44 | 18 | 23075655 | 11664424 | 8186 | ACGTACTTTGRTTGTTTAAAA | 0.757 | 0.714 | 0.362 | 0.546 | 0.886 |
| 44 | 18 | 23081711 | 16943386 | 8187 | CAAGTGTTATYTCCCAGGGGT | 0.046 | 0.047 | 0.375 | 0.765 | 0.509 |
| 44 | 18 | 23087741 | 16943406 | 8188 | AGCTCTTACARGAATTGAGAA | 0.000 | 0.000 | 0.516 | 0.518 | 0.698 |
| 44 | 18 | 23102007 | 1426868 | 8189 | AGAAATTATCRAAGTTCAAGT | 0.883 | 0.881 | 0.588 | 0.563 | 0.816 |
| 44 | 18 | 23112652 | 9954770 | 8190 | AACATTAGCAYATTTTTAACA | 0.412 | 0.405 | 0.628 | 0.579 | 0.878 |
| 44 | 18 | 23126621 | 11661123 | 8191 | GTTTCCACTGYGTTGTTTTAT | 0.857 | 0.870 | 0.865 | 0.745 | 0.694 |
| 44 | 18 | 23133906 | 1714636 | 8192 | AATCTTCCAAMTCTTACAGTA | 0.122 | 0.124 | 0.502 | 0.998 | 0.533 |
| 44 | 18 | 23154769 | 17714238 | 8193 | ATCCTTTGATMCTGATGCTAG | 1.009 | 1.024 | 0.915 | 0.924 | 0.607 |
| 44 | 18 | 23166163 | 7236330 | 8194 | ACTACAATTTWCTTGGATTAT | 0.112 | 0.109 | 0.754 | 0.684 | 0.461 |
| 44 | 18 | 23172924 | 1474135 | 8195 | ATAAAGAAATRAAACTGGACA | 1.171 | 1.180 | 0.942 | 0.641 | 0.484 |
| 44 | 18 | 23179814 | 4800802 | 8196 | AAAGAGGGTCRATGGCTTTTA | 0.602 | 0.596 | 0.500 | 0.451 | 0.454 |
| 44 | 18 | 23189662 | 10502490 | 8197 | GACAATCCTGWTGGTGCCACT | 0.449 | 0.417 | 0.561 | 0.462 | 0.471 |
| 44 | 18 | 23202749 | 10502491 | 8198 | CATGAAAAAGWGCAAAAAATT | 0.232 | 0.231 | 0.243 | 0.207 | 0.508 |
| 44 | 18 | 23210271 | 1519143 | 8199 | CCTTACCTGARAGCCTGAATG | 0.256 | 0.256 | 0.113 | 0.269 | 0.366 |
| 44 | 18 | 23215939 | 10502492 | 8200 | TTAGAGAAAASTTAAGATGAG | 0.343 | 0.326 | 0.059 | 0.225 | 0.363 |
| 44 | 18 | 23231527 | 1401466 | 8201 | TTATTCCTGCKGCCAGAGCTG | 0.136 | 0.142 | 0.069 | 0.213 | 0.356 |
| 44 | 18 | 23245216 | 1401462 | 8202 | TAATTCCAGGYGGCATACTAA | 0.146 | 0.140 | 0.276 | 0.186 | 0.434 |
| 44 | 18 | 23253013 | 6508486 | 8203 | CTGGAGAGCCRCTTGAGAGTA | 0.311 | 0.315 | 0.344 | 0.151 | 0.212 |
| 44 | 18 | 23258373 | 9956638 | 8204 | CATTCTAGCCYACCCCATGAA | 0.989 | 0.990 | 0.373 | 0.315 | 0.167 |
| 45 | 18 | 61324317 | 17777796 | 8205 | TCTTTTAGATKTGTGGGTATA | 0.424 | 0.397 | 1.755 | 1.246 | 0.910 |
| 45 | 18 | 61336000 | 7233342 | 8206 | ATTTAATATGRCTTTTTTGCT | 0.042 | 0.039 | 2.018 | 1.331 | 1.239 |
| 45 | 18 | 61348906 | 17700174 | 8207 | GGTGAAAATAYGCAACATTCT | 4.001 | 3.392 | 2.394 | 1.260 | 1.374 |
| 45 | 18 | 61359787 | 17074963 | 8208 | ACTAACATTAYTGCTACTTAA | 0.000 | 0.000 | 2.434 | 1.608 | 1.289 |
| 45 | 18 | 61364918 | 9636017 | 8209 | CTTCTTTTCAYCTTACTTACT | 0.482 | 0.504 | 2.536 | 2.156 | 1.442 |
| 45 | 18 | 61385547 | 10503100 | 8210 | CATGAATTGAYACACATATTT | 0.075 | 0.079 | 0.432 | 1.951 | 1.562 |
| 45 | 18 | 61394512 | 17778833 | 8211 | CTAAAAACTGYAACTCAAGTT | 0.147 | 0.152 | 0.813 | 1.853 | 1.649 |
| 45 | 18 | 61412020 | 17779022 | 8212 | CTAAATTGCTRTCTCAACTTG | 1.084 | 1.076 | 0.811 | 0.767 | 1.476 |
| 45 | 18 | 61422482 | 13380899 | 8213 | AGTGACTTGCYAACTTTATTG | 1.336 | 1.378 | 0.938 | 0.790 | 1.361 |
| 45 | 18 | 61430893 | 17701273 | 8214 | GTTTGTATTTYGATTGTGGAT | 0.576 | 0.595 | 1.101 | 0.834 | 1.360 |
| 45 | 18 | 61441000 | — | 8215 | TGAAATAAATSTTGCCACGTT | 0.502 | 0.503 | 0.963 | 0.806 | 0.501 |
| 45 | 18 | 61451163 | 17075033 | 8216 | CTTCTCCACCYTATCATTTTT | 0.000 | 0.000 | 0.654 | 0.793 | 0.626 |
| 45 | 18 | 61460357 | 12604790 | 8217 | AATTTTGTCTSTTCAACCTTA | 0.559 | 0.555 | 0.395 | 0.552 | 0.559 |
| 45 | 18 | 61469322 | 10871590 | 8218 | TCACATGGTARATTTCCGTTA | 0.700 | 0.743 | 0.249 | 0.278 | 0.556 |
| 45 | 18 | 61475252 | 1942822 | 8219 | TAGATGCAAAWAGTTCATTAG | 0.004 | 0.004 | 0.200 | 0.381 | 0.540 |
| 45 | 18 | 61479028 | 1942273 | 8220 | TAAAGGACATRTATGGATTGC | 0.162 | 0.162 | 0.127 | 0.304 | 0.423 |
| 45 | 18 | 61496107 | — | 8221 | TTCTCAGAAAYAGTATCTTTT | 0.213 | 0.217 | 0.210 | 0.246 | 0.221 |
| 45 | 18 | 61504971 | 17708381 | 8222 | CATGATTATCMATTTACTAAC | 0.244 | 0.259 | 0.289 | 0.160 | 0.152 |
| 45 | 18 | 61515106 | 17075147 | 8223 | TCATGTTTGAWTGCAGAGAAA | 1.082 | 1.059 | 0.296 | 0.096 | 0.195 |
| 45 | 18 | 61522755 | 10515997 | 8224 | TATTCATGTCKATTAAGGAGT | 0.189 | 0.198 | 0.254 | 0.114 | 0.226 |
| 45 | 18 | 61543876 | 17781554 | 8225 | TTGTAAGAGGMCACTGACCTA | 0.103 | 0.104 | 0.288 | 0.101 | 0.323 |
| 45 | 18 | 61549981 | 9962601 | 8226 | GGAGTGTCTGSTTGCCTCTCC | 0.054 | 0.055 | 0.014 | 0.237 | 0.451 |
| 45 | 18 | 61556288 | 7226378 | 8227 | AAGCCCTTAARGAAATATAGA | 0.000 | 0.000 | 0.007 | 0.296 | 0.480 |
| 45 | 18 | 61565394 | 10084067 | 8228 | CTTTGTTGTAYTCCCACATGT | 0.017 | 0.017 | 0.170 | 0.344 | 0.668 |
| 45 | 18 | 61582244 | 12961301 | 8229 | GTATTTTTACRCTTCACTCTG | 0.151 | 0.153 | 0.355 | 0.634 | 0.876 |
| 45 | 18 | 61590121 | 8092259 | 8230 | TTTGTCTTTTKTAAGATAAAT | 0.836 | 0.845 | 0.603 | 0.683 | 1.381 |
| 45 | 18 | 61595696 | 1484725 | 8231 | CTCCAAACGCRAAGCAAATTT | 0.559 | 0.557 | 0.987 | 0.973 | 1.576 |
| 45 | 18 | 61601643 | 990388 | 8232 | ATGCACATATRCAACATAGGA | 1.242 | 1.200 | 1.089 | 1.167 | 2.017 |
| 45 | 18 | 61607583 | 17783081 | 8233 | CTGTTTAGGCRTCTGTAATTG | 1.621 | 1.613 | 1.363 | 1.683 | 2.112 |
| 45 | 18 | 61613910 | 8097065 | 8234 | ATAAATGCACYTGTCCTGAAG | 0.195 | 0.198 | 1.782 | 2.159 | 2.335 |
| 45 | 18 | 61621115 | 4334398 | 8235 | TCGGTGAACAYGAAGCTCTAA | 1.218 | 1.162 | 2.446 | 2.730 | 2.366 |
| 45 | 18 | 61627292 | 11659738 | 8236 | TTGAAGTCCCRTGCATGTATT | 1.382 | 1.349 | 2.981 | 2.988 | 2.505 |
| 45 | 18 | 61634277 | 4144764 | 8237 | CCTATGGAGAYAAAGCCATAC | 2.719 | 2.775 | 3.835 | 3.149 | 2.740 |
| 45 | 18 | 61635487 | 6566179 | 8238 | TAAGAGGTATYGTCATAACAA | 2.465 | 2.467 | 3.723 | 3.111 | 2.819 |
| 45 | 18 | 61672858 | 1484711 | 8239 | AGTTCAAGGCYTTTTAGCTTT | 2.709 | 2.640 | 3.723 | 3.124 | 2.905 |
| 45 | 18 | 61678763 | 12967018 | 8240 | ATTTTGAATCRTATGTTCGAA | 0.775 | 0.770 | 2.852 | 3.254 | 2.942 |
| 45 | 18 | 61686997 | 2587410 | 8241 | GGAAACTTTTYTTCTATATTA | 1.996 | 1.843 | 2.131 | 3.173 | 2.840 |
| 45 | 18 | 61697973 | 2628210 | 8242 | TTATCTTTGASAGTTGCTGAA | 0.706 | 0.685 | 1.762 | 2.516 | 2.957 |
| 45 | 18 | 61706203 | 7229176 | 8243 | AGTAAAAGAGSCTTAAAAATG | 1.541 | 1.516 | 1.515 | 1.738 | 2.934 |
| 45 | 18 | 61717877 | — | 8244 | TACTTCCCATYACTACATCTC | 1.234 | 1.224 | 0.962 | 1.245 | 2.911 |
| 45 | 18 | 61725718 | 17075520 | 8245 | TAATGGATTGYAAATGGAAAG | 0.072 | 0.072 | 0.789 | 1.472 | 2.206 |
| 45 | 18 | 61738544 | 183260 | 8246 | AAGAGCTACAWATTAATCACA | 0.174 | 0.214 | 0.410 | 1.243 | 1.677 |
| 45 | 18 | 61742429 | 2628245 | 8247 | CTATAAATAASCATGCCCTAA | 0.262 | 0.262 | 0.684 | 1.301 | 1.134 |
| 45 | 18 | 61758901 | 9949357 | 8248 | TTTCTAAAACRTATTTGAGCT | 0.525 | 0.495 | 1.098 | 0.880 | 0.985 |
| 45 | 18 | 61764669 | 6566183 | 8249 | ATAAAGTCCAYGGTGGATTCT | 1.949 | 1.818 | 1.225 | 0.480 | 0.739 |
| 45 | 18 | 61771354 | 17075606 | 8250 | TGGCCCCTTTYGCCAACGCCA | 0.807 | 0.792 | 1.158 | 0.466 | 0.660 |
| 45 | 18 | 61776985 | 6566186 | 8251 | ATAAAATCGTRGGATTAGAGA | 0.463 | 0.455 | 0.879 | 0.422 | 0.403 |
| 45 | 18 | 61783512 | 2541870 | 8252 | ATGTGCCAGTWTGCTTTTATT | 0.142 | 0.143 | 0.117 | 0.510 | 0.431 |
| 45 | 18 | 61790957 | 9945649 | 8253 | TCCTGACTACSAGGGCAAGGA | 0.009 | 0.009 | 0.010 | 0.439 | 0.473 |
| 45 | 18 | 61792527 | 2541874 | 8254 | AAAGGTCACCRTCATCTAGCA | 0.041 | 0.040 | 0.028 | 0.090 | 0.528 |
| 45 | 18 | 61804445 | 9962706 | 8255 | AACTATGCTAYGACACCATTA | 0.034 | 0.033 | 0.044 | 0.187 | 0.498 |
| 45 | 18 | 61816573 | 9949391 | 8256 | ATAATTGTTAYGCATTTTTGA | 0.631 | 0.613 | 0.083 | 0.162 | 0.626 |
| 45 | 18 | 61829609 | 2541743 | 8257 | TATATAGGTCRATGAAACAGA | 0.250 | 0.250 | 0.504 | 0.219 | 0.301 |
| 45 | 18 | 61836360 | 2715320 | 8258 | GCAGCAAAATRCCTTGGTCAC | 0.259 | 0.247 | 0.628 | 0.237 | 0.186 |
| 45 | 18 | 61842726 | 2706582 | 8259 | ACTGCAAACCYCAATCTTATG | 1.417 | 1.390 | 0.580 | 0.524 | 0.258 |
| 45 | 18 | 61852405 | 10513920 | 8260 | TAGGCGAGTCRAGATAGAAAA | 0.271 | 0.273 | 0.511 | 0.610 | 0.253 |
| 45 | 18 | 61859712 | 2541778 | 8261 | AAAAACATTCWTCAAATTAAT | 0.000 | 0.000 | 0.897 | 0.459 | 0.280 |
| 45 | 18 | 61867908 | 4891639 | 8262 | AATGAGATTCYTTGGAAGATG | 0.123 | 0.119 | 0.404 | 0.629 | 0.285 |
| 45 | 18 | 61874530 | 2715297 | 8263 | AAACATAATAMAATCATTTTC | 1.134 | 1.128 | 0.294 | 0.554 | 0.528 |
| 45 | 18 | 61881775 | 995160 | 8264 | TACTGAAATTYTAAAGTAGAA | 0.294 | 0.286 | 0.496 | 0.246 | 0.492 |
| 45 | 18 | 61888583 | 2706614 | 8265 | AGTACCTATTRAGATGATGAC | 0.017 | 0.017 | 0.456 | 0.193 | 0.628 |
| 45 | 18 | 61897220 | 2541809 | 8266 | CCTTCTTTCAYAGGCACAGAG | 0.882 | 0.889 | 0.140 | 0.431 | 0.609 |
| 45 | 18 | 61905727 | 12966332 | 8267 | GAATGGACTCYGCATTAACCC | 0.002 | 0.001 | 0.075 | 0.506 | 0.434 |
| 45 | 18 | 61916445 | 17279167 | 8268 | GGTTGCTTTCYTTACTTTAAT | 0.205 | 0.196 | 0.519 | 0.516 | 0.392 |
| 45 | 18 | 61924877 | 2058842 | 8269 | ATTGAAATACRAAAGAATACT | 0.008 | 0.008 | 0.376 | 0.476 | 0.429 |
| 45 | 18 | 61932124 | 12607652 | 8270 | GTAAAGCAAASGACTATAGTA | 1.520 | 1.455 | 0.766 | 0.513 | 0.559 |
| 45 | 18 | 61945465 | 4439860 | 8271 | GTGGTAGCCAKAATAATGGTC | 0.422 | 0.393 | 0.727 | 0.361 | 0.510 |
| 46 | 22 | 16451100 | 1296819 | 8272 | GCAATAGACTMCCACTAGAAG | 0.035 | 0.036 | 0.014 | 0.000 | 0.006 |
| 46 | 22 | 16457814 | 5747252 | 8273 | TCTACAGTTCYTTCATTTAGG | 0.215 | 0.218 | 0.008 | 0.053 | 0.045 |
| 46 | 22 | 16469866 | 5747268 | 8274 | TCTGGTCTCAYTGGTCCCTGT | 0.301 | 0.317 | 0.007 | 0.077 | 0.061 |
| 46 | 22 | 16482520 | — | 8275 | ATACCCACTCRTCTTAGTGCA | 0.036 | 0.036 | 0.332 | 0.114 | 0.064 |
| 46 | 22 | 16492758 | 5747302 | 8276 | AAAGTCAAATRATTTCTATAA | 0.039 | 0.039 | 0.393 | 0.317 | 0.092 |
| 46 | 22 | 16506100 | 713701 | 8277 | ATCAGGTTTTRTAAGAAAATT | 1.523 | 1.474 | 0.394 | 0.380 | 0.129 |
| 46 | 22 | 16518718 | 8140916 | 8278 | GCCAACATTTYATTTTTTAAG | 0.381 | 0.386 | 0.824 | 0.339 | 0.327 |
| 46 | 22 | 16534258 | 5992786 | 8279 | TAACATTTTCRAGTTTTTATG | 0.287 | 0.287 | 0.919 | 0.367 | 0.521 |
| 46 | 22 | 16546790 | 2587076 | 8280 | TTCCTCTAGTSGTGACATGAA | 1.042 | 1.007 | 0.283 | 0.472 | 0.845 |
| 46 | 22 | 16558565 | 2587082 | 8281 | ATAGCTCTTARTTTTTAAAAG | 0.239 | 0.241 | 0.298 | 0.898 | 1.017 |
| 46 | 22 | 16568261 | 5992801 | 8282 | CAATATATTAYGCCAAAGTTG | 0.013 | 0.013 | 0.353 | 0.731 | 0.932 |
| 46 | 22 | 16574940 | 3788279 | 8283 | CTCTTTGAAGRTAGAGATTGT | 0.405 | 0.405 | 0.509 | 1.031 | 1.075 |
| 46 | 22 | 16581805 | 1008378 | 8284 | AGAACATCCTYGATGCTTACC | 0.428 | 0.430 | 0.826 | 1.252 | 1.197 |
| 46 | 22 | 16587611 | 8919 | 8285 | TCTAGATGAARAAATGCAACT | 1.436 | 1.374 | 1.471 | 0.982 | 0.960 |
| 46 | 22 | 16593018 | 181382 | 8286 | TGCAAAAACAYGTTCTCCAGA | 0.935 | 0.936 | 1.785 | 1.130 | 0.887 |
| 46 | 22 | 16598709 | 181396 | 8287 | CTCTGCTTAARTGACAAGTCA | 1.429 | 1.438 | 1.623 | 1.403 | 0.988 |
| 46 | 22 | 16603741 | 5747339 | 8288 | GAGGAAAAACYGTATGCAAAA | 0.987 | 0.961 | 1.290 | 1.573 | 0.736 |
| 46 | 22 | 16625446 | 2268786 | 8289 | CGGAGCAGCCRCCAGTGTGTC | 0.042 | 0.059 | 1.065 | 1.341 | 2.107 |
| 46 | 22 | 16639385 | 5992838 | 8290 | GCCCTCCCAGRGTCTGGCTGT | 0.000 | 0.000 | 0.514 | 0.989 | 2.082 |
| 46 | 22 | 16652682 | 426276 | 8291 | TGCCACCTCCWGGGACTGGTG | 0.000 | 0.000 | 0.020 | 0.591 | 2.101 |
| 46 | 22 | 16665930 | 454799 | 8292 | CTGAGTTTCAYTCTATGGGAA | 0.000 | 0.000 | 0.092 | 2.055 | 1.928 |
| 46 | 22 | 16678946 | 2305006 | 8293 | TCGCTGAGTCKGCTCCCAGTT | 0.093 | 0.092 | 0.061 | 1.445 | 1.490 |
| 46 | 22 | 16690645 | 450796 | 8294 | AGTGAGAGGCYGGGGGAAATC | 0.000 | 0.000 | 2.881 | 1.547 | 1.137 |
| 46 | 22 | 16703965 | 2111546 | 8295 | GCTGGATTGCMAAGCCGAAGT | 0.161 | 0.163 | 1.776 | 1.337 | 0.809 |
| 46 | 22 | 16729337 | 5992128 | 8296 | TCCTTCTTCAKGGAGCCCTGC | 4.215 | 3.869 | 1.952 | 1.095 | 0.640 |
| 46 | 22 | 16743102 | 2289718 | 8297 | CGAACGCTGGRGTCTGTGACA | 0.136 | 0.136 | 1.688 | 0.882 | 0.710 |
| 46 | 22 | 16756240 | 5992895 | 8298 | AAACTGTATCSGCAGTGACAC | 0.228 | 0.229 | 1.753 | 0.993 | 0.758 |
| 46 | 22 | 16769795 | 2083882 | 8299 | ACCCATAAGCRTGAATTTGTT | 0.126 | 0.121 | 0.006 | 0.929 | 0.710 |
| 46 | 22 | 16782233 | 2401413 | 8300 | AAGCTTCCATSCCTGCTGGCA | 0.086 | 0.088 | 0.028 | 1.006 | 0.589 |
| 46 | 22 | 16788529 | 4484121 | 8301 | CCCTGCTGGGYACACACAGAT | 0.016 | 0.017 | 0.049 | 0.073 | 0.745 |
| 46 | 22 | 16808838 | 8135939 | 8302 | TCGCCAAAGGRAAAACTCAGG | 0.407 | 0.404 | 0.087 | 0.088 | 1.067 |
| 46 | 22 | 16821385 | 445583 | 8303 | GGCCTTCCCCRAAGGCAGACA | 0.365 | 0.363 | 0.272 | 0.062 | 1.170 |
| 46 | 22 | 16833681 | 365219 | 8304 | AGCACTGCCCSATTTTCTTGG | 0.394 | 0.365 | 0.352 | 0.131 | 0.245 |
| 46 | 22 | 16860571 | 424765 | 8305 | TTGGTAAAGAYGAATCCTGTC | 0.710 | 0.700 | 0.207 | 0.522 | 0.603 |
| 46 | 22 | 16873861 | 7284666 | 8306 | TTGGCAATCASTCTTCTATTT | 0.285 | 0.273 | 0.328 | 0.649 | 1.020 |
| 46 | 22 | 16886836 | 107321 | 8307 | AACATGATTAYTGACTTTATG | 0.019 | 0.020 | 0.924 | 0.709 | 1.086 |
| 46 | 22 | 16897768 | 465101 | 8308 | ATCTCAGCCTYTTTTGCAAGT | 0.724 | 0.691 | 0.731 | 1.445 | 2.176 |
| 46 | 22 | 16901343 | 462904 | 8309 | TGACCGCTCGKGATGTTAAGC | 1.575 | 1.681 | 0.882 | 2.104 | 2.144 |
| 46 | 22 | 16920365 | 5992986 | 8310 | CACATATGTTKGTCTTCAGAG | 0.386 | 0.379 | 2.175 | 1.789 | 1.828 |
| 46 | 22 | 16927573 | 1005195 | 8311 | TGTGTAGCTAYGCATGCTCAT | 0.578 | 0.572 | 2.404 | 3.065 | 2.166 |
| 46 | 22 | 16937442 | 464541 | 8312 | CCCTCCAGATYGGATTCTTTC | 1.752 | 1.733 | 1.418 | 2.755 | 1.906 |
| 46 | 22 | 16944707 | 361818 | 8313 | CGAGGTTTCCRTCACATAGCT | 1.558 | 1.605 | 2.457 | 2.144 | 2.079 |
| 46 | 22 | 16951892 | 361594 | 8314 | GAAAACCGATYTGTATGGTCC | 0.166 | 0.161 | 2.034 | 1.862 | 2.117 |
| 46 | 22 | 16962268 | 361809 | 8315 | ATGGGTGAATRTATGTAAGGT | 2.159 | 2.143 | 1.292 | 1.631 | 2.287 |
| 46 | 22 | 16971003 | 362043 | 8316 | GCTCTGTGGAKTTGATAGGTG | 0.045 | 0.044 | 1.046 | 1.916 | 2.051 |
| 46 | 22 | 16979683 | 466456 | 8317 | GACTGGGCACRAAGTATATGC | 0.363 | 0.356 | 1.095 | 1.488 | 1.503 |
| 46 | 22 | 16989428 | 2540620 | 8318 | CTGACCTTATMTCCAATCCTC | 1.105 | 1.100 | 0.724 | 1.113 | 1.519 |
| 46 | 22 | 17003269 | 361534 | 8319 | TTTCTTGTACYGAATATTTAC | 0.313 | 0.302 | 0.966 | 1.170 | 1.523 |
| 47 | X | 80030757 | 5959057 | 8320 | TTTTTCTCCAWGTTTATTTAA | 0.890 | 0.934 | 0.612 | 1.319 | 1.147 |
| 47 | X | 80036134 | 5959059 | 8321 | TCAAAGCAGAYGTTTAGCATC | 0.497 | 0.503 | 1.550 | 1.234 | 1.251 |
| 47 | X | 80050301 | 1166617 | 8322 | CAAAAGCCTTYCGGCGTGGGC | 0.721 | 0.727 | 1.468 | 1.269 | 1.194 |
| 47 | X | 80058048 | 1166623 | 8323 | CCAACAACCAKGACAGGCAAA | 2.394 | 2.471 | 1.487 | 1.445 | 1.178 |
| 47 | X | 80067200 | 1166634 | 8324 | AGACACTCAGRTTTCAAAGAC | 1.165 | 1.183 | 1.607 | 1.415 | 1.031 |
| 47 | X | 80103135 | 1166676 | 8325 | TTGCACATATYTATGAGGACA | 1.075 | 1.081 | 1.728 | 1.304 | 1.058 |
| 47 | X | 80134248 | 5913394 | 8326 | ACCCCCTGACSCTTCCTCAAT | 0.522 | 0.520 | 1.074 | 1.375 | 1.215 |
| 47 | X | 80171239 | — | 8327 | ACTTCTGAACMGGAACATGTA | 0.577 | 0.611 | 0.601 | 1.364 | 1.128 |
| 47 | X | 80191739 | 5959816 | 8328 | GGAAGATTTAYACCAGGAACT | 0.471 | 0.462 | 0.222 | 0.685 | 1.005 |
| 47 | X | 80225545 | 5959831 | 8329 | ATTTATTGTAYAGGCCTTCAA | 0.088 | 0.085 | 0.155 | 0.586 | 1.097 |
| 47 | X | 80235899 | 17333035 | 8330 | TCCTTTTTATYTAATTTACAG | 0.164 | 0.081 | 0.140 | 0.255 | 1.074 |
| 47 | X | 80253754 | 5913411 | 8331 | GTGATGCCCARTGTTCTGCTT | 0.333 | 0.319 | 0.260 | 0.146 | 1.552 |
| 47 | X | 80277451 | 12014025 | 8332 | ATTTCACTAGRCAGAAGTGTG | 0.547 | 0.538 | 0.252 | 0.153 | 1.587 |
| 47 | X | 80297073 | 2027279 | 8333 | GTTGAGTATCRGAAAAGTGAA | 0.831 | 0.869 | 0.213 | 0.204 | 1.337 |
| 47 | X | 80305924 | 17328569 | 8334 | ATTTGGGACTRTGTTCATGCA | 0.071 | 0.069 | 0.353 | 1.612 | 1.229 |
| 47 | X | 80320875 | 5912483 | 8335 | CTGCCTGGAGRATCTGTCTGA | 0.078 | 0.075 | 0.409 | 1.851 | 2.437 |
| 47 | X | 80325649 | 5959088 | 8336 | CTGTGTGCATRCTGCCTGTGT | 0.627 | 0.648 | 1.946 | 1.817 | 2.156 |
| 47 | X | 80337509 | 2039717 | 8337 | CGAGCAACTTRGAAAGACAAA | 0.613 | 0.611 | 2.115 | 1.592 | 2.354 |
| 47 | X | 80348471 | 4826212 | 8338 | CTGTTTGATTRGATGACTCAT | 3.799 | 3.542 | 2.237 | 2.736 | 2.840 |
| 47 | X | 80359374 | 1044828 | 8339 | TTGTAATTTCYTATCGTAATT | 0.960 | 0.961 | 2.029 | 2.483 | 2.840 |
| 47 | X | 80387275 | 5959099 | 8340 | TTTTAATCTARCTCTTTATTC | 0.167 | 0.173 | 3.004 | 2.576 | 3.024 |
| 47 | X | 80400947 | 1117471 | 8341 | GTCTTAATTTWTCCTACATTT | 0.423 | 0.433 | 1.899 | 2.858 | 3.133 |
| 47 | X | 80411297 | 5912496 | 8342 | AAGGATGGATRTAACGGAGAG | 4.215 | 4.422 | 2.227 | 2.807 | 3.102 |
| 47 | X | 80417482 | 5913445 | 8343 | TGTGGGGAATRGAAATTGAGA | 0.478 | 0.444 | 2.848 | 2.723 | 3.511 |
| 47 | X | 80426557 | 5913447 | 8344 | ACGAGCATTARCTGTTTTATA | 1.753 | 1.742 | 3.159 | 3.397 | 3.415 |
| 47 | X | 80435314 | 6616745 | 8345 | TTGAAGGACAYAGATGGATAC | 3.205 | 3.604 | 2.633 | 3.225 | 3.454 |
| 47 | X | 80452285 | — | 8346 | GGAATATGCTRTTGTCCCTGC | 0.907 | 0.904 | 3.474 | 3.735 | 3.130 |
| 47 | X | 80459676 | 5959880 | 8347 | ATTGGAATTGRTCTTTGAAGA | 2.992 | 2.874 | 3.447 | 3.214 | 3.392 |
| 47 | X | 80466557 | — | 8348 | GCTGTTATTTWTTTCCCTAGC | 1.576 | 1.504 | 3.835 | 3.380 | 3.308 |
| 47 | X | 80478736 | 2806640 | 8349 | TATTTGCAGAYATTTTTTTTT | 2.412 | 2.255 | 3.441 | 3.348 | 3.237 |
| 47 | X | 80492353 | 2602598 | 8350 | GTAGTGTGCTRGATAACAGAG | 4.015 | 4.091 | 3.280 | 3.237 | 2.899 |
| 47 | X | 80506071 | 2444578 | 8351 | CAGAACCAAARGGCTGACTAC | 1.070 | 1.058 | 2.677 | 2.903 | 2.903 |
| 47 | X | 80511331 | 2806658 | 8352 | GATATACAAAKTCATCTTTAA | 1.383 | 1.417 | 2.574 | 2.668 | 2.860 |
| 47 | X | 80554116 | 10521395 | 8353 | GTACCCTTAAYGGCCAGCTAA | 1.279 | 1.299 | 1.633 | 2.426 | 2.630 |
| 47 | X | 80583233 | 5912517 | 8354 | AGTACCAAATRTAAAGACATC | 2.093 | 2.071 | 1.907 | 2.222 | 2.579 |
| 47 | X | 80597043 | 4529587 | 8355 | ATGGGGAAGAYAGTCTCTATA | 0.908 | 0.889 | 1.934 | 1.829 | 2.362 |
| 47 | X | 80606131 | 5913519 | 8356 | GATCGTTCACYTTTTGTGTGT | 2.126 | 2.054 | 1.955 | 1.928 | 2.183 |

TABLE 3

| SNP markers found to be associated with Longevity in Fine Mapping studies in the QFP, Individual SNP markers genotyped in the genome wide | | | | | | | | | | | | | |
|---|---|---|---|---|---|---|---|---|---|---|---|---|---|
| scan are presented in each row of the table. The corresponding region ID and chromosome is presented as identified in Table 1 (columns 1 and | | | | | | | | | | | | | |
| 2). The coordinate of the SNP according to the NCBI genome assembly build 35 is indicated in column 3. The RS# column corresponds to | | | | | | | | | | | | | |
| the NCBI dbSNP identifier for the SNP (column 4). The Seq ID is the unique numerical identifier for this SNP in the sequence listing for this | | | | | | | | | | | | | |
| patent, and is indicated in column 5. Column 6, labeled Flanking Sequence, corresponds to 21 bp of nucleotide sequence centered at the | | | | | | | | | | | | | |
| SNP, which is coded using the standard degenerate naming system. The remainder of the table lists −log10 p values for association of the | | | | | | | | | | | | | |
| indicated haplotype centered at the corresponding SNP with the disease as described in the text, using LDSTATS V4 and Single Type. Values | | | | | | | | | | | | | |
| for the association of single markers, as well as 3, 5, 7, 9, 11 marker haplotype windows are shown for LDSTATS V4 analysis (see text for | | | | | | | | | | | | | |
| explanation of statistical calculations). The last two columns represent the −log10 P values for single marker genotype and allele | | | | | | | | | | | | | |
| association using Single Type analysis (see EXAMPLE section for description of analysis). | | | | | | | | | | | | | |
| | | | | | | | | | | | | Single Type | |
| | | | | | | LDSTATS v4 | | | | | | Single Genotype | Single Allele |
| Region ID | Chr | B35 Position | RS# | Seq ID | Flanking Sequence | Single Marker | W03 | W05 | W07 | W09 | W11 | Likelihood Ratio | Likelihood Ratio |
| 1 | 1 | 173369115 | 10753133 | 8357 | AATTAAGCAGRTTAATGCAGT | 0.097 | — | — | — | — | — | 0.054 | 0.090 |
| 1 | 1 | 173391904 | 726252 | 4850 | CTGTCTCTCAYGCCTTCCTTG | 0.299 | 0.007 | — | — | — | — | 0.161 | 0.323 |
| 1 | 1 | 173424493 | 1995651 | 4853 | CCCAAAAAAAWTTCTATCATT | 0.199 | 0.261 | 0.555 | — | — | — | 0.092 | 0.225 |
| 1 | 1 | 173455634 | 1886766 | 8358 | AGATTTGACCRTAAGCTCTGG | 0.963 | 0.615 | 0.680 | 0.351 | — | — | 0.561 | 0.939 |
| 1 | 1 | 173475519 | 1325599 | 4858 | TTATTTCCCCRCATGAAAATG | 1.615 | 1.183 | 0.530 | — | — | — | 1.660 | 1.658 |
| 1 | 1 | 173491681 | 10913254 | 4860 | TTTAAGCAGAMAGGCAAAAAC | — | — | — | — | — | — | — | — |
| 1 | 1 | 173522159 | 10732997 | 4863 | ATTATTTGAARTAGATTTGAA | 0.000 | 0.993 | — | — | — | — | 0.006 | 0.004 |
| 1 | 1 | 173539000 | 16850237 | 4865 | AGATTAGAAGYGACCCTTGTT | — | — | — | — | — | — | — | — |
| 1 | 1 | 173564270 | 6425398 | 8359 | CTCCAAGTCTYCTCTTGAATG | 0.185 | — | — | — | — | — | 0.064 | 0.196 |
| 2 | 1 | 225626459 | — | 4909 | CACGCCTTTCYAGCCTCACAC | — | — | — | — | — | — | — | — |
| 2 | 1 | 225645683 | 342785 | 4911 | AGGAGAAGAGMCTTCAAACTG | — | — | — | — | — | — | — | — |
| 2 | 1 | 225660552 | — | 4912 | CACATCTACCRACCATCTGAC | — | — | — | — | — | — | — | — |
| 2 | 1 | 225667166 | — | 4913 | AGTGGAGGTCRTTAGGAGAGG | — | — | — | — | — | — | — | — |
| 2 | 1 | 225673850 | 342818 | 4914 | ACATTTAAATYATCCAGTCTG | 0.221 | — | — | — | — | — | 0.137 | 0.228 |
| 2 | 1 | 225680359 | 7526949 | 4915 | CCCATTGTTCRTTTTTGGAAT | — | — | — | — | — | — | — | — |
| 2 | 1 | 225692972 | 16849638 | 4917 | TCATCTCTTAWGCAACTGAAC | 1.515 | 0.568 | — | — | — | — | 1.066 | 1.523 |
| 2 | 1 | 225703254 | — | 4918 | GCACATTTCAYCAAGCTGATA | — | — | — | — | — | — | — | — |
| 2 | 1 | 225722322 | — | 4919 | TGAATATAATYATAAACTGAG | — | — | — | — | — | — | — | — |
| 2 | 1 | 225741733 | 237778 | 8360 | TGAAAGCCCCRTCCTTATCAG | 0.452 | 1.629 | — | — | — | — | 0.193 | 0.463 |
| 2 | 1 | 225759122 | 237819 | 8361 | TCTATTGTTCYATACTGTCTC | 0.194 | — | — | — | — | — | 0.084 | 0.203 |
| 3 | 1 | 227250818 | 1202518 | 8362 | GTCCTTGGGCRTGCGTTTTTA | 0.214 | — | — | — | — | — | 0.108 | 0.223 |
| 3 | 1 | 227258633 | 1202525 | 4971 | TGAGGCTGCASGGAGGCCAGA | 0.179 | 0.010 | — | — | — | — | 0.033 | 0.148 |
| 3 | 1 | 227265670 | 1202534 | 4972 | GTTTTCCCCCRAAAAGGTTGG | 0.143 | 0.188 | 0.896 | — | — | — | 0.137 | 0.174 |
| 3 | 1 | 227270465 | — | 4973 | ATGGCCCCGTRAGGTTAGCGG | 0.554 | 1.653 | 0.953 | 0.754 | — | — | 0.347 | 0.571 |
| 3 | 1 | 227281064 | 6673201 | 4974 | AGGTGTCGCARAGATTAAGGC | — | — | — | — | — | — | — | — |
| 3 | 1 | 227291324 | 16852841 | 4975 | TGCTCAGCTCYAAAAACTCCA | 3.105 | 2.010 | 1.278 | 1.076 | 0.274 | — | 3.684 | 3.192 |
| 3 | 1 | 227301328 | 1999903 | 4976 | CTCTGAGGAAYGAAGACTTAG | — | — | — | — | — | — | — | — |
| 3 | 1 | 227307747 | 16852927 | 4977 | ACCGGCTCCTYTCCTTTTGCT | 0.910 | 2.235 | 1.429 | 0.465 | — | — | 0.549 | 0.929 |
| 3 | 1 | 227315639 | 1202589 | 8363 | TGCTTTAACCRAGTCAAACTG | 0.085 | 0.660 | 1.550 | — | — | — | 0.079 | 0.103 |
| 3 | 1 | 227320088 | 1202594 | 4978 | TTTTTCCAATKGTAGAGAGAG | 0.051 | 0.527 | — | — | — | — | 0.046 | 0.035 |
| 3 | 1 | 227327915 | 1202603 | 8364 | TTTCCTACCTKTTTCCTCTGT | — | — | — | — | — | — | — | — |
| 3 | 1 | 227334767 | 6704527 | 4980 | TTACTTCCTGKTAAGAGGTCT | 1.087 | — | — | — | — | — | 0.690 | 1.089 |
| 4 | 1 | 236300211 | 1110615 | 5016 | TCTGAAAATCRCCATCTGTAA | 0.604 | — | — | — | — | — | 0.314 | 0.647 |
| 4 | 1 | 236311513 | 6429161 | 5017 | TCTGCATAGAWATCTAATTCA | — | — | — | — | — | — | — | — |
| 4 | 1 | 236324113 | 477507 | 5018 | CACCAACTAGRTTGAAAATTG | — | — | — | — | — | — | — | — |
| 4 | 1 | 236339319 | 613228 | 5019 | TCCTTTTAATWTAAATATGTT | — | — | — | — | — | — | — | — |
| 4 | 1 | 236356440 | 682355 | 8365 | CCAGACAGCCRTCTTCTTTAG | 0.473 | 0.489 | — | — | — | — | 0.205 | 0.473 |
| 4 | 1 | 236365881 | 17598757 | 5020 | ACTCTCTCTAYGTCTCTATAT | 0.519 | 0.270 | 0.224 | — | — | — | 0.376 | 0.533 |
| 4 | 1 | 236375371 | 672548 | 8366 | TCTGTTAAAAYATTTAATACC | 0.520 | 0.423 | 0.444 | 0.318 | — | — | 1.087 | 0.556 |
| 4 | 1 | 236381514 | 10495448 | 5022 | TTTTAATCCCRTAGAGCCAGA | 0.049 | 0.078 | 0.514 | 0.295 | — | — | 0.044 | 0.041 |
| 4 | 1 | 236391685 | 10926012 | 5023 | TCGTCTCAAARTAGTCAAGTA | 0.067 | 0.149 | 0.012 | — | — | — | 0.304 | 0.066 |
| 4 | 1 | 236403350 | 4659554 | 5024 | AGGGGCTCACRTCTCAGTTGC | 0.189 | 0.004 | — | — | — | — | 0.094 | 0.187 |
| 4 | 1 | 236410617 | 1953666 | 8367 | CTAATCTCAASTGTTAGGAGG | 0.165 | — | — | — | — | — | 0.302 | 0.140 |
| 5 | 2 | 21157019 | 3791981 | 5075 | TGATCTCTCCRGAGCTATTGT | 0.293 | — | — | — | — | — | 0.264 | 0.280 |
| 5 | 2 | 21162692 | 570877 | 5076 | CAAAATGTCTKGATTTCATTG | 0.319 | 0.013 | — | — | — | — | 0.449 | 0.309 |
| 5 | 2 | 21168039 | 1864423 | 8368 | CATCTTCTTAKTACCTGGAAG | — | — | — | — | — | — | — | — |
| 5 | 2 | 21171264 | 520354 | 8369 | TTGTGCAGAARAGAACAGAAG | 0.175 | 0.068 | 0.068 | — | — | — | 0.071 | 0.203 |
| 5 | 2 | 21175552 | 1367117 | 8370 | CTCTTTCAGGRTGCACTGGCT | 0.181 | 0.172 | 0.134 | 0.028 | — | — | 0.133 | 0.187 |
| 5 | 2 | 21179434 | 512535 | 8371 | CGTTTCCTTCYCTTCTAGGCA | 0.410 | 0.239 | 0.051 | 0.072 | 0.035 | — | 0.230 | 0.440 |
| 5 | 2 | 21185142 | 7575840 | 8372 | AGCCAGGAATKGTCAGTACTG | 0.011 | 0.086 | 0.080 | 0.028 | — | — | 0.005 | 0.020 |
| 5 | 2 | 21189574 | 6548010 | 8373 | ACACATATTCRTAAGTTAAAC | 0.056 | 0.001 | 0.021 | — | — | — | 0.014 | 0.036 |
| 5 | 2 | 21194554 | 7590135 | 8374 | CTCCAAGCCCRGCTGCTAAAA | 0.049 | 0.035 | — | — | — | — | 0.014 | 0.036 |
| 5 | 2 | 21199203 | — | 5080 | GCCGTCCATCYATCCGTTACT | — | — | — | — | — | — | — | — |
| 5 | 2 | 21204658 | 594677 | 5081 | ACTGAGTAATYGTCTAATGAA | 0.248 | — | — | — | — | — | 0.222 | 0.263 |
| 6 | 2 | 49991254 | 10495984 | 5116 | AATGATTTATSTGTTCCTATG | — | — | — | — | — | — | — | — |
| 6 | 2 | 50004404 | 17490406 | 5118 | GTTTATGTGAYTGTGACATTC | 2.251 | — | — | — | — | — | 1.910 | 2.269 |
| 6 | 2 | 50010410 | 1981797 | 5119 | AATTTTTCTCYTTAATATATC | 0.754 | 1.531 | — | — | — | — | 1.024 | 0.720 |
| 6 | 2 | 50029185 | 12998574 | 8375 | AGTTAACAAARGGGTTTATGT | 2.157 | 1.822 | 1.063 | — | — | — | 2.915 | 2.113 |
| 6 | 2 | 50037491 | 1156742 | 5120 | AATGGTATAARAATATTAGGG | 2.751 | 1.768 | 1.604 | 1.406 | — | — | 2.900 | 2.666 |
| 6 | 2 | 50048374 | 17039425 | 5121 | CTATTTGCCTRTGATTTTTAA | — | — | — | — | — | — | — | — |
| 6 | 2 | 50055972 | 12465974 | 5122 | ATCAAAGATTYCAATTCTTGG | 2.415 | 2.041 | 1.806 | 1.257 | 1.048 | — | 1.820 | 2.414 |
| 6 | 2 | 50064931 | 10495987 | 5123 | TTGGAAGCTAMTGATTATTCT | 3.062 | 2.226 | 2.193 | 1.645 | 0.992 | — | 2.424 | 3.061 |
| 6 | 2 | 50077180 | 6758043 | 5124 | ATCATTGCTTRTGTAATCTGA | 0.891 | 2.615 | 1.518 | 1.155 | — | — | 1.181 | 0.893 |
| 6 | 2 | 50087864 | 10166360 | 8376 | AATAAAGGCTSTAAAACTTAA | 0.060 | 0.770 | 1.283 | — | — | — | 1.485 | 0.048 |
| 6 | 2 | 50095750 | 6713560 | 5127 | AGCTTTTAACRGTTCTCAAAG | 1.394 | 0.601 | — | — | — | — | 1.237 | 1.392 |
| 6 | 2 | 50101389 | 17039577 | 8377 | TGACCTTTCAYATTTCCCATG | 1.391 | — | — | — | — | — | 1.438 | 1.378 |
| 7 | 2 | 51025393 | 10490172 | 5237 | TTGCTGCTTASAAACAAATTC | 0.100 | — | — | — | — | — | 0.118 | 0.105 |
| 7 | 2 | 51032119 | 17041091 | 5238 | GGACAGACAAKTATTTTGTAG | — | — | — | — | — | — | — | — |
| 7 | 2 | 51039440 | 17041095 | 5239 | TAGGGCTCTTMAATAGTAGGA | 1.289 | 1.369 | — | — | — | — | 0.865 | 1.314 |
| 7 | 2 | 51046992 | 1160595 | 5240 | TGTTTCATGTYTTAAAGTGGG | 1.748 | 0.763 | 0.579 | — | — | — | 1.595 | 1.789 |
| 7 | 2 | 51052993 | 1003017 | 5241 | TGGACATACAMTAATGTATCT | 1.419 | 1.044 | 0.219 | 0.880 | — | — | 1.404 | 1.462 |
| 7 | 2 | 51058686 | 741421 | 5242 | CTGTCATTCAKTCTAACTTGC | — | — | — | — | — | — | — | — |
| 7 | 2 | 51076203 | 1541602 | 5244 | CTATCAGTAGYATGGGAATGA | 0.990 | 0.593 | 0.634 | 0.307 | 0.654 | — | 0.746 | 1.047 |
| 7 | 2 | 51083805 | 7579976 | 5245 | CATTCTAACAYCTACAAAGAG | 0.741 | 0.654 | 0.450 | 0.554 | — | — | 0.834 | 0.786 |
| 7 | 2 | 51096961 | 10205578 | 5247 | TGGAAAGATTKTAATCAGAAA | 0.979 | 0.530 | 0.319 | — | — | — | 0.817 | 1.020 |
| 7 | 2 | 51107252 | 10174398 | 5249 | TCTGTACCCAYGCTCTCTCCA | 0.723 | 0.577 | — | — | — | — | 0.577 | 0.764 |
| 7 | 2 | 51126792 | 17041161 | 5251 | AGAAAATCCAYAAGTCTAAAT | — | — | — | — | — | — | — | — |
| 7 | 2 | 51140357 | 10195460 | 8378 | TTAAGCATACRTTTTTCCACC | 0.999 | — | — | — | — | — | 1.018 | 1.048 |
| 8 | 2 | 121763582 | 2580344 | 8379 | TATTTCTCCCYCAAGTTCCAG | 1.115 | — | — | — | — | — | 0.740 | 1.142 |
| 8 | 2 | 121795575 | 2713226 | 8380 | ACGAGCAGGTKGTCACCCACC | 0.843 | 0.519 | — | — | — | — | 0.495 | 0.821 |
| 8 | 2 | 121809417 | 2713250 | 8381 | GGAGCACTGCYGGACCAAATG | 0.597 | 0.556 | 0.452 | — | — | — | 0.328 | 0.596 |
| 8 | 2 | 121833968 | 12479320 | 5309 | TGGTGCCAATRTCTCTAAAAG | 1.026 | 0.669 | 0.408 | 0.176 | — | — | 0.622 | 1.064 |
| 8 | 2 | 121870433 | 10496566 | 5312 | ATATAGAATAWGTTTCAGCCC | 0.994 | 0.681 | 0.499 | 0.416 | 0.129 | — | 0.646 | 1.101 |
| 8 | 2 | 121886006 | 10191223 | 5313 | ACCACAGAAAKGGAGAAAAAT | 0.162 | 0.691 | 1.152 | 0.591 | 0.336 | 0.122 | 0.136 | 0.186 |
| 8 | 2 | 121905688 | 13032411 | 8382 | AATATTTTATKTGAAAAGTCA | 0.187 | 1.531 | 0.876 | 0.821 | 0.634 | — | 0.136 | 0.186 |
| 8 | 2 | 121923235 | 1975305 | 8383 | CATCTAAGACYATACCTAGAA | 1.780 | 0.871 | 0.792 | 0.830 | — | — | 1.309 | 1.866 |
| 8 | 2 | 121940587 | 7587659 | 8384 | CATGTTGCTTKTAAGAACAAA | 0.652 | 0.928 | 0.846 | — | — | — | 1.196 | 0.662 |
| 8 | 2 | 121982154 | 6704587 | 8385 | GAGCAACTGCRAATAATTCCC | 0.493 | 0.429 | — | — | — | — | 0.214 | 0.480 |
| 8 | 2 | 121995577 | 10221922 | 8386 | CACACTGGCTKTAGGGAAATG | — | — | — | — | — | — | — | — |
| 8 | 2 | 122017375 | 2164797 | 5325 | AGCCCCCAGCSCGCCAACAGG | 0.423 | — | — | — | — | — | 0.212 | 0.409 |
| 9 | 2 | 127668493 | 13006847 | 5367 | GCTGAACAGGYCAAACTATTT | 0.844 | — | — | — | — | — | 0.444 | 0.818 |
| 9 | 2 | 127692604 | 6710496 | 5368 | AAGCCCCCATMCCAGAAATAA | 1.163 | 0.474 | — | — | — | — | 0.887 | 1.149 |
| 9 | 2 | 127723916 | 6430936 | 5369 | CCCGAACAGGMTTTTGTTCAC | 1.174 | 0.407 | 0.234 | — | — | — | 0.873 | 1.188 |
| 9 | 2 | 127729734 | 4662717 | 5370 | GTTCCTGGGGYTTGCACTGAT | 1.263 | 1.012 | 0.831 | 0.313 | — | — | 0.825 | 1.298 |
| 9 | 2 | 127749557 | 4150474 | 5371 | ACAAACCCACMAAGAAAACAG | 1.562 | 1.586 | 1.193 | 0.684 | — | — | 1.194 | 1.572 |
| 9 | 2 | 127754774 | 4150454 | 5372 | CCATCACTTTYAGACCTGTCC | 2.194 | 1.619 | 1.471 | — | — | — | 1.661 | 2.272 |
| 9 | 2 | 127776393 | 4233584 | 5373 | CCTGGATTTCYTACTCACTGT | — | — | — | — | — | — | — | — |
| 9 | 2 | 127802584 | 12613413 | 5374 | TTTTGATGGTYCACATGCCAA | 0.122 | 2.011 | — | — | — | — | 0.150 | 0.133 |
| 9 | 2 | 127828431 | 6714840 | 5376 | GCAAAGGATCWGTTTCCAAGT | — | — | — | — | — | — | — | — |
| 9 | 2 | 127853791 | 4662724 | 8387 | GCCTAGAACAYAGAAAATTAC | 0.112 | — | — | — | — | — | 0.150 | 0.133 |
| 10 | 2 | 162628815 | 1913807 | 8388 | GAAAAAATAAYCCATGGAGAA | 0.084 | — | — | — | — | — | 0.050 | 0.118 |
| 10 | 2 | 162644128 | 4500960 | 5442 | TCCAGCAGCAYGTTACTGTCT | 1.285 | 1.337 | — | — | — | — | 0.878 | 1.321 |
| 10 | 2 | 162653508 | 4664442 | 5443 | CTTTTAATGGRTCCTATGTAA | 1.592 | 2.139 | 1.964 | — | — | — | 1.104 | 1.601 |
| 10 | 2 | 162657263 | 4295021 | 5444 | CCTGTTGATTKTTTAGCTGAA | 2.402 | 2.135 | 1.590 | 2.753 | — | — | 1.935 | 2.486 |
| 10 | 2 | 162671072 | 1861979 | 5445 | TTAAAGCCTGYAAGCACCAAA | 2.620 | 1.464 | 2.590 | 3.182 | 2.397 | — | 2.079 | 2.610 |
| 10 | 2 | 162680415 | 2287509 | 5446 | CCTCCTTGTTKCTCTCCAAAT | 2.467 | 2.729 | 3.236 | 2.786 | 1.484 | — | 2.171 | 2.527 |
| 10 | 2 | 162694710 | 1014445 | 5448 | TCTGTAAAGCRCTCTCATTTC | 2.677 | 4.279 | 3.467 | 1.629 | — | — | 2.160 | 2.676 |
| 10 | 2 | 162709469 | 2300755 | 8389 | CCCCTGTCCTRTATCAGTGGT | 3.325 | 2.591 | 2.307 | — | — | — | 2.922 | 3.347 |
| 10 | 2 | 162720273 | 10930040 | 8390 | TAACTATTTTRAAAGCTTATA | 2.463 | 1.679 | — | — | — | — | 2.034 | 2.509 |
| 10 | 2 | 162749424 | 12469968 | 5453 | CTCTACCTCARTTATACATCC | 0.898 | — | — | — | — | — | 0.568 | 0.914 |
| 10 | 2 | 162762494 | 7593348 | 8391 | TCTCACTGGGKTTTGCCAGAG | — | — | — | — | — | — | — | — |
| 12 | 3 | 7122158 | 17046783 | 5601 | TCATGAATTTYGGCATTTTTT | — | — | — | — | — | — | — | — |
| 12 | 3 | 7128652 | 6778030 | 5602 | TTGACTATCTRTGAAAACTGT | 0.125 | — | — | — | — | — | 0.057 | 0.143 |
| 12 | 3 | 7136316 | 17234935 | 5603 | TCATAATCTASGACTGGATAT | 0.774 | 2.621 | — | — | — | — | 0.421 | 0.782 |
| 12 | 3 | 7148002 | 1878164 | 5605 | ATGACACTTCRTCTACTTGAA | 0.757 | 1.727 | 2.848 | — | — | — | 0.447 | 0.792 |
| 12 | 3 | 7155162 | 6804466 | 5606 | ACTTTATAGGYATACTGGTAG | 0.765 | 1.857 | 1.801 | 2.675 | — | — | 0.511 | 0.811 |
| 12 | 3 | 7162872 | 17234969 | 5607 | ACAACCATGAYCCTGACCTTG | 2.545 | 1.710 | 1.752 | 1.496 | 2.027 | — | 1.960 | 2.598 |
| 12 | 3 | 7168821 | 7623514 | 5608 | TCCTCAAGAAMGACTTGCATT | 0.188 | 1.610 | 1.593 | 1.407 | 1.277 | — | 0.106 | 0.204 |
| 12 | 3 | 7174495 | 6443093 | 5609 | AATGTAGAAGYGGCAGAAGAC | 0.417 | 0.298 | 1.135 | 1.219 | — | — | 0.287 | 0.422 |
| 12 | 3 | 7187512 | 17235018 | 5610 | AAACCTACATSATTCTGTATG | — | — | — | — | — | — | — | — |
| 12 | 3 | 7193175 | 1400166 | 5611 | TCATCCTTTCRTCTTTATTCA | 0.291 | 0.206 | 0.184 | — | — | — | 0.116 | 0.335 |
| 12 | 3 | 7198739 | 13082571 | 5612 | GGCTAAAGAAYAGTACAAACC | 0.000 | 0.356 | — | — | — | — | 0.035 | 0.000 |
| 12 | 3 | 7204619 | 11708019 | 5613 | CAGTTGTCTCRATGCCTAGTA | 0.000 | — | — | — | — | — | 0.035 | 0.000 |
| 13 | 3 | 33009602 | 4075736 | 5725 | TTTCATAAAGRAGAGAAATAA | 0.437 | — | — | — | — | — | 0.401 | 0.442 |
| 13 | 3 | 33046631 | 6762132 | 5728 | CAGGAGGTATRAACAGTGCTG | 1.158 | 0.828 | — | — | — | — | 0.765 | 1.173 |
| 13 | 3 | 33062204 | 6780220 | 5729 | CTGCCTTAATMGGGGCTATAG | 1.079 | 2.419 | 1.095 | — | — | — | 0.916 | 1.093 |
| 13 | 3 | 33074192 | 4438612 | 5730 | GTGTCCTGCCRAGGTGGGAGG | 2.594 | 1.997 | 1.144 | 1.432 | — | — | 2.137 | 2.651 |
| 13 | 3 | 33104989 | 9858362 | 8392 | TTCTCTATACMCTAAAAATGA | 1.953 | 1.070 | 1.537 | 1.316 | 1.836 | — | 1.419 | 1.923 |
| 13 | 3 | 33121193 | 4578976 | 5732 | TCCAGAGATAYGAGTTGGGAC | 0.535 | 0.770 | 0.365 | 1.623 | 1.983 | 0.880 | 0.279 | 0.582 |
| 13 | 3 | 33133916 | 4678686 | 5733 | TGCAGCCTCARGTGCATCATA | 1.299 | 0.612 | 1.108 | 1.210 | 1.924 | — | 0.930 | 1.308 |
| 13 | 3 | 33151828 | 7652193 | 5734 | ACTGTGGTTTRCAAAGTATAG | 0.859 | 1.157 | 1.526 | 1.003 | — | — | 0.583 | 0.891 |
| 13 | 3 | 33179004 | 4678490 | 5735 | TTCTTTGAACWGACGAGTAAG | 0.049 | 1.195 | 0.992 | — | — | — | 0.101 | 0.051 |
| 13 | 3 | 33191738 | 4465894 | 5736 | CTGGCAGAATRCCTGGAATAG | 0.576 | 0.168 | — | — | — | — | 0.481 | 0.601 |
| 13 | 3 | 33217346 | 4678763 | 8393 | TTATGGAAGGYAGAACTTGCA | 0.123 | — | — | — | — | — | 0.198 | 0.150 |
| 14 | 3 | 40246701 | 1317217 | 5803 | ATGATTAATAKCTGGAGAAAG | 2.772 | — | — | — | — | — | 2.185 | 2.837 |
| 14 | 3 | 40284229 | 9861194 | 8394 | CTGATTCAATRTGTAAAGGGC | 3.148 | 1.703 | — | — | — | — | 2.337 | 3.157 |
| 14 | 3 | 40314657 | 4974047 | 8395 | TCGAATGGTAKGCTTAACCTC | 3.104 | 1.467 | 1.179 | — | — | — | 3.310 | 3.035 |
| 14 | 3 | 40349555 | 4974067 | 5811 | AAGTCTAACAYGCAGCTCTGA | 3.097 | 2.030 | 1.870 | 1.540 | — | — | 2.697 | 3.130 |
| 14 | 3 | 40379774 | 10510708 | 5815 | TGAATCAAGTYGGTCTACAAT | 2.631 | 2.530 | 2.299 | 1.508 | 1.147 | — | 2.493 | 2.661 |
| 14 | 3 | 40398626 | 9854493 | 5816 | CTCTAGTCTAYAGTTAGCTAT | 3.587 | 3.052 | 1.764 | 1.819 | — | — | 3.110 | 3.624 |
| 14 | 3 | 40404187 | 2305521 | 5817 | ATGTTCTGAARAAAAGCCCCG | 2.481 | 2.471 | 2.174 | — | — | — | 2.255 | 2.496 |
| 14 | 3 | 40417584 | — | 5818 | CTGGAGAGGTSCTGAGATGTT | — | — | — | — | — | — | — | — |
| 14 | 3 | 40424355 | 6783755 | 5819 | TCTAAGAAGTYAGACAGACAT | — | — | — | — | — | — | — | — |
| 14 | 3 | 40431030 | 7645864 | 5820 | GCAAAAGGGAYTGTGGTGTAC | — | — | — | — | — | — | — | — |
| 14 | 3 | 40468222 | 6801859 | 8396 | AAAAGATCTAKAACTTCCCCA | 2.353 | 2.675 | — | — | — | — | 1.964 | 2.381 |
| 14 | 3 | 40496977 | 4571217 | 8397 | AATCTTTACARTGATGACTGC | 3.418 | — | — | — | — | — | 3.016 | 3.499 |
| 15 | 4 | 77256496 | 10031733 | 8398 | TATGACCTTAYGAGGCATTCT | 0.078 | — | — | — | — | — | 0.071 | 0.073 |
| 15 | 4 | 77291919 | 867562 | 5870 | CTGTGTATTTRAGTTGTTTCC | 1.084 | 0.594 | — | — | — | — | 0.648 | 1.038 |
| 15 | 4 | 77320175 | 7684461 | 8399 | GAGAGGCAGAKAGATAACTGT | 0.130 | 0.914 | 1.015 | — | — | — | 0.098 | 0.155 |
| 15 | 4 | 77352642 | 4304003 | 5874 | GACTCCTGACRAGGTACCTGA | 0.091 | 1.438 | 1.131 | 0.923 | — | — | 0.098 | 0.102 |
| 15 | 4 | 77384429 | 10028141 | 5878 | AAATATTATTRACACTTCAGA | — | — | — | — | — | — | — | — |
| 15 | 4 | 77402183 | 11733489 | 5880 | TATATCTACGRTGATGCCTCT | 1.838 | 1.256 | 1.270 | 1.441 | — | — | 1.441 | 1.949 |
| 15 | 4 | 77430723 | 6824251 | 5882 | CACATTCCAAMATAACAGAAC | 1.919 | 0.907 | 1.838 | — | — | — | 1.443 | 1.932 |
| 15 | 4 | 77456563 | 17001573 | 5885 | TTATTTGGGCRTGTAGCTATG | 0.016 | 1.718 | — | — | — | — | 0.006 | 0.033 |
| 15 | 4 | 77488248 | 17001659 | 5889 | GCCCTGACTGYGGCCAGTTCT | 1.662 | — | — | — | — | — | 1.130 | 1.605 |
| 15 | 4 | 77516879 | 1441922 | 8400 | TCAAGAAGTARGCAGGTAAAG | — | — | — | — | — | — | — | — |
| 15 | 4 | 77550724 | 1441911 | 5893 | AGATTTAAGTKCCTAAGTTGG | — | — | — | — | — | — | — | — |
| 16 | 4 | 101082500 | 10031644 | 5916 | AGTAGGGAATKTATTTGCACA | 0.147 | — | — | — | — | — | 0.619 | 0.124 |
| 16 | 4 | 101095575 | 2282588 | 5917 | GAAAACAAGTWGGTTGAGAAA | 2.159 | 0.939 | — | — | — | — | 1.688 | 2.164 |
| 16 | 4 | 101103675 | 10031708 | 5918 | CAGAGCTTCTYGCATCATTTC | 0.000 | 1.703 | 1.067 | — | — | — | 0.103 | 0.029 |
| 16 | 4 | 101110330 | 2162386 | 5919 | AACAGGTTATMTTTCAAGGCC | — | — | — | — | — | — | — | — |
| 16 | 4 | 101123160 | 4699387 | 5921 | TTTGACGACASCTACCGCTAT | 0.550 | 0.270 | 1.681 | 1.127 | — | — | 0.263 | 0.563 |
| 16 | 4 | 101129781 | 4699767 | 5922 | GCACCTTATCYGGGCCCAAGT | 0.551 | 0.359 | 0.371 | 1.363 | 1.263 | — | 0.263 | 0.563 |
| 16 | 4 | 101130928 | 11935615 | 5923 | CAGCTTGTTCRGGTCATGTTC | 0.445 | 0.575 | 0.662 | 1.549 | 1.591 | 1.034 | 0.234 | 0.443 |
| 16 | 4 | 101149661 | 6829912 | 5924 | GCTGAGCCTAYGGTCTGCTCT | 0.515 | 0.743 | 1.170 | 0.967 | 1.402 | 1.660 | 0.328 | 0.567 |
| 16 | 4 | 101158451 | 11723286 | 5925 | TTTATTTACARAGGCTATTTT | 0.842 | 1.002 | 1.180 | 0.783 | 1.190 | — | 0.510 | 0.831 |
| 16 | 4 | 101183482 | 17029641 | 5928 | TCTTACACATRAAGTATATGG | 1.318 | 1.174 | 0.826 | 1.559 | — | — | 1.385 | 1.299 |
| 16 | 4 | 101209057 | 17613664 | 5930 | AGTGTGTTCTYCAAAAAGATT | 0.064 | 0.986 | 1.268 | — | — | — | 0.391 | 0.067 |
| 16 | 4 | 101233826 | 11736218 | 8401 | GCTAAGAAGAYTAAACAGGGA | 0.658 | 1.841 | — | — | — | — | 0.359 | 0.661 |
| 10 | 4 | 101258022 | 7689566 | 5933 | AGAGCTATCARTTTATAGAGC | 2.484 | — | — | — | — | — | 1.955 | 2.505 |
| 17 | 4 | 126670525 | 1509292 | 8402 | GTTCCCACACMAAAACTAGGA | — | — | — | — | — | — | — | — |
| 17 | 4 | 126681071 | 12501179 | 8403 | TATTTTCATGRGTCTGAATTG | 1.184 | — | — | — | — | — | 0.960 | 1.200 |
| 17 | 4 | 126691002 | 4623022 | 8404 | TGGCCAAAGTYACTTTTTAGA | 0.380 | 0.459 | — | — | — | — | 0.523 | 0.388 |
| 17 | 4 | 126705065 | 6842220 | 5973 | TAATGAAAATYGCTTGAAAAC | 0.364 | 0.347 | 0.737 | — | — | — | 0.472 | 0.365 |
| 17 | 4 | 126717061 | 1395241 | 8405 | TTTCTAGTGCRTGTTAAAGAT | 0.985 | 0.833 | 0.815 | 0.894 | — | — | 0.861 | 1.012 |
| 17 | 4 | 126726483 | 13108706 | 5976 | CCTTATGGCARCTTTTCTGCA | — | — | — | — | — | — | — | — |
| 17 | 4 | 126738202 | 17009708 | 5978 | GAAAGTCTGCRGATACCATGG | — | — | — | — | — | — | — | — |
| 17 | 4 | 126752525 | 12506486 | 5980 | GGACATGGGAKCAAGAGGTGA | 1.623 | 0.944 | 1.353 | 0.549 | 0.213 | — | 1.218 | 1.693 |
| 17 | 4 | 126766922 | 7686537 | 8406 | TGAAAGTGTAYGAAAGGTTAA | 0.461 | 1.111 | 0.697 | 0.435 | — | — | 0.460 | 0.465 |
| 17 | 4 | 126781219 | 1395228 | 8407 | TTTTCCATTAMTACCTAACAC | 1.639 | 0.832 | 0.746 | — | — | — | 1.113 | 1.642 |
| 17 | 4 | 126796200 | 10034622 | 8408 | TCATACAACARGTGTGTGTTT | 0.814 | 1.120 | — | — | — | — | 0.569 | 0.785 |
| 17 | 4 | 126804720 | 17009923 | 5988 | TGGGGAAAAGRGATTAAAAAT | 0.372 | — | — | — | — | — | 0.143 | 0.391 |
| 18 | 4 | 143639478 | 1497397 | 6062 | ATAATAGTCAMGATGATGGTT | 0.283 | — | — | — | — | — | 0.194 | 0.307 |
| 18 | 4 | 143657608 | 6828156 | 8409 | GAGGTCCCACKACCCTTGTTG | 0.736 | 0.184 | — | — | — | — | 0.443 | 0.790 |
| 18 | 4 | 143679095 | 1497401 | 8410 | ATTTAAACTAYCTTAAAACAC | 0.059 | 0.063 | 0.149 | — | — | — | 0.253 | 0.055 |
| 18 | 4 | 143700188 | 3775711 | 8411 | TCTAGCACAGYCCCAGAGGCA | 0.425 | 0.640 | 0.786 | 0.617 | — | — | 0.203 | 0.485 |
| 18 | 4 | 143718806 | 931637 | 6070 | CTGTCAGAACRTAAATTTGAT | 2.137 | 1.465 | 1.318 | 0.924 | 0.654 | — | 1.832 | 2.178 |
| 18 | 4 | 143739539 | 17016163 | 6071 | GATGCCCTTTSCAACACATAT | 2.923 | 2.092 | 1.419 | 1.495 | — | — | 2.319 | 2.946 |
| 18 | 4 | 143764438 | 3844178 | 6073 | TTAAGAAATARGGAACTGAAC | 3.342 | 2.535 | 1.754 | — | — | — | 2.797 | 3.350 |
| 18 | 4 | 143785479 | 2322795 | 8412 | GAGCTAGAAASATGGGGAGAT | 3.352 | 2.057 | — | — | — | — | 2.875 | 3.369 |
| 18 | 4 | 143808180 | 975136 | 8413 | GCAGATAAGAYAATTCAGGTA | 0.194 | — | — | — | — | — | 0.232 | 0.200 |
| 19 | 6 | 27117491 | 7775041 | 6096 | TTGACTATATWCATGCATTCT | 0.400 | — | — | — | — | — | 0.179 | 0.416 |
| 19 | 6 | 27131673 | 10946899 | 8414 | GGAGTAAAATWACTTTCCTAA | 0.809 | 0.603 | — | — | — | — | 0.760 | 0.865 |
| 19 | 6 | 27138903 | 3922717 | 8415 | CTTTATATCTRTGCAAGAACA | 1.251 | 0.931 | 1.457 | — | — | — | 1.325 | 1.311 |
| 19 | 6 | 27150237 | 6916301 | 8416 | AATCCTTCTAYTTTCTAGTCC | 0.015 | 1.108 | 1.332 | 1.013 | — | — | 0.142 | 0.041 |
| 19 | 6 | 27155692 | 994691 | 8417 | GTATAAAGAAWGCCTTGAGAA | 1.927 | 1.469 | 1.051 | 1.086 | 0.969 | — | 1.416 | 1.965 |
| 19 | 6 | 27162476 | 2142685 | 6098 | TTTATGTTCTYTTGAAGATGA | 0.875 | 1.410 | 1.048 | 0.977 | 0.924 | 0.929 | 0.510 | 0.902 |
| 19 | 6 | 27172468 | — | 6099 | GTATTCACTCRAGATGTTAGT | — | — | — | — | — | — | — | — |
| 19 | 6 | 27183462 | 9348752 | 6100 | AGAATGTTCAYAGATATTTCT | 0.594 | 0.764 | 1.718 | 1.106 | 0.762 | — | 0.330 | 0.629 |
| 19 | 6 | 27193204 | 12209456 | 8418 | CTAAGTCATGYCCAGACTCCT | 0.963 | 1.028 | 1.272 | 1.222 | — | — | 0.603 | 1.016 |
| 19 | 6 | 27201800 | 6456768 | 6101 | TGGAGGATGGSATAGTAAAGA | 1.332 | 0.815 | 0.662 | — | — | — | 0.890 | 1.372 |
| 19 | 6 | 27211559 | 2022272 | 6102 | ACAATTAAATRTACAGTGGTT | 1.161 | 0.668 | — | — | — | — | 0.793 | 1.241 |
| 19 | 6 | 27222031 | 9393790 | 6103 | TACTCAGTGTKATAAGATCAT | 1.039 | — | — | — | — | — | 0.649 | 1.058 |
| 20 | 6 | 152767168 | 6905339 | 6172 | AACAATGTGAYACAAACTGTA | 0.984 | — | — | — | — | — | 0.963 | 1.023 |
| 20 | 6 | 152789820 | 214955 | 6175 | GAAAAGGTGAYGCAAAAATTT | 1.050 | 1.247 | — | — | — | — | 0.662 | 1.093 |
| 20 | 6 | 152810619 | 549981 | 6176 | TCCTCAGGCCYTTTCTTTTCC | 2.519 | 1.758 | 1.509 | — | — | — | 1.935 | 2.545 |
| 20 | 6 | 152838247 | 6909684 | 6179 | TATATTAAAAYGAAATGGACT | 2.079 | 2.393 | 0.958 | 0.811 | — | — | 1.750 | 2.121 |
| 20 | 6 | 152861412 | 17082700 | 6182 | GTATCCCATAYAGCAAGAAGT | 0.217 | 1.413 | 1.564 | 0.969 | 1.005 | — | 0.131 | 0.207 |
| 20 | 6 | 152868292 | 214970 | 6183 | GTGACACAGTRTCTATACTAT | 1.682 | 1.192 | 1.408 | 1.506 | 0.629 | 0.721 | 1.188 | 1.732 |
| 20 | 6 | 152875047 | 579464 | 6184 | CTTAACTTCCRGATTCCACAG | 1.683 | 1.719 | 1.232 | 0.782 | 0.964 | 0.559 | 1.188 | 1.732 |
| 20 | 6 | 152887970 | 7755437 | 6185 | ATTCATAATTMCTTATATTGA | 1.696 | 1.534 | 0.964 | 0.668 | 0.906 | — | 1.188 | 1.732 |
| 20 | 6 | 152898003 | 9397512 | 6186 | TGTTGCCATAYCACTGTGTAG | 1.600 | 1.007 | 0.842 | 1.625 | — | — | 1.092 | 1.634 |
| 20 | 6 | 152913124 | 9479327 | 6187 | AATTTATTTTYTGGATGATTA | — | — | — | — | — | — | — | — |
| 20 | 6 | 152925152 | 11155857 | 6188 | CCAACAAAATRGACTTACATG | 0.498 | 0.856 | 1.639 | — | — | — | 0.511 | 0.532 |
| 20 | 6 | 152934088 | 17699371 | 6189 | CTGTTAGACAYGACACAGTCT | 0.051 | 0.435 | — | — | — | — | 0.476 | 0.072 |
| 20 | 6 | 152940828 | 11155858 | 6190 | AGCCATATACRGTCATTAAAA | 0.797 | — | — | — | — | — | 0.479 | 0.845 |
| 21 | 7 | 30540960 | 255160 | 6249 | ATGAAAGCTGYCTTGCCTAAG | — | — | — | — | — | — | — | — |
| 21 | 7 | 30565088 | 4723010 | 6251 | CTACAGCTTCRATGGCAGCCC | — | — | — | — | — | — | — | — |
| 21 | 7 | 30579153 | 3901848 | 6252 | TGTCCTTCCARTGTAAAGTCC | 0.985 | — | — | — | — | — | 0.591 | 0.977 |
| 21 | 7 | 30602279 | 11768076 | 6254 | TGTGGCCTTTRAGTACCTTGG | 0.000 | 1.691 | — | — | — | — | 0.492 | 0.007 |
| 21 | 7 | 30610787 | 7803974 | 6255 | AGCAGGGCTAYGTTCTGAGAA | 1.896 | 2.000 | 1.618 | — | — | — | 1.478 | 1.861 |
| 21 | 7 | 30618224 | 1990011 | 6256 | TGGATATGTCSAGTGGAGCTT | 0.000 | 2.493 | 1.570 | 0.754 | — | — | 0.612 | 0.009 |
| 21 | 7 | 30629046 | 10237363 | 6257 | CCCGGGTCTCRTATGCCAAAT | 2.753 | 2.182 | 1.118 | 1.180 | 2.561 | — | 2.281 | 2.819 |
| 21 | 7 | 30641902 | 17159487 | 6258 | GTAAGTTAACKAAGGTCTCCA | 1.232 | 1.492 | 1.742 | 2.383 | 2.564 | — | 0.814 | 1.222 |
| 21 | 7 | 30649882 | 10229281 | 6259 | TGGGAATAGARCTGCTTCATC | 0.450 | 1.687 | 2.864 | 3.310 | — | — | 0.399 | 0.463 |
| 21 | 7 | 30671937 | 17159526 | 6260 | TCAGGTTGTTSCAATGTCCCA | 3.299 | 3.169 | 2.385 | — | — | — | 2.786 | 3.305 |
| 21 | 7 | 30682866 | 17159567 | 6261 | CAAAGTTATAMATCTCTTAAA | — | — | — | — | — | — | — | — |
| 21 | 7 | 30688503 | 10216063 | 6262 | GCATTACAACRTGTGCCAGGT | 3.574 | 3.298 | — | — | — | — | 2.875 | 3.586 |
| 21 | 7 | 30710418 | 1000597 | 6264 | AGGACAGTTTYGTAATCAGGA | 0.322 | — | — | — | — | — | 0.127 | 0.345 |
| 22 | 7 | 33466478 | 7811071 | 6364 | CAGCATTTGTRAGGCCAGGTG | 0.368 | — | — | — | — | — | 0.269 | 0.376 |
| 22 | 7 | 33474415 | 2034513 | 8419 | CCATGATTAAYGTACAACATA | 0.206 | 0.483 | — | — | — | — | 0.140 | 0.218 |
| 22 | 7 | 33478828 | 7796551 | 8420 | TTTGAAATATYTAATTGATGA | — | — | — | — | — | — | — | — |
| 22 | 7 | 33484293 | 4723309 | 6366 | TATCAGTTTTRTAAGTAAGCA | 0.000 | 0.243 | 0.364 | — | — | — | 0.091 | 0.014 |
| 22 | 7 | 33491746 | 6966497 | 6367 | CTTGTGGTGAYCAGCACTACT | 0.000 | 0.670 | 0.275 | 0.345 | — | — | 0.082 | 0.009 |
| 22 | 7 | 33501439 | 1545449 | 8421 | TTTTTCTTCGYAAGTTCTTAA | 0.285 | 0.638 | 0.639 | 0.175 | 0.082 | — | 0.369 | 0.299 |
| 22 | 7 | 33505602 | 1376350 | 6369 | AACTGAGAGCRATTGTGGCAA | — | — | — | — | — | — | — | — |
| 22 | 7 | 33511585 | 921413 | 8422 | TCTGACAGAARAATGTGGGGT | 0.468 | 0.548 | 0.423 | 0.294 | 0.055 | — | 0.305 | 0.503 |
| 22 | 7 | 33516650 | 7796839 | 8423 | CTAGTGCAATYTCATTTTGAC | 0.509 | 0.181 | 0.401 | 0.168 | — | — | 0.341 | 0.494 |
| 22 | 7 | 33524601 | 17810887 | 8424 | CTGCAAAGCTRGGAACCAGGG | 0.365 | 0.380 | 0.216 | — | — | — | 0.225 | 0.384 |
| 22 | 7 | 33530235 | 6943676 | 8425 | GTTGCTGAGGSACTTTACTCC | 0.790 | 0.439 | — | — | — | — | 0.880 | 0.812 |
| 22 | 7 | 33537029 | 3779238 | 8426 | GCCATTGCTAYGATTTCTCCC | 1.052 | — | — | — | — | — | 0.803 | 1.074 |
| 23 | 7 | 127442525 | 2060736 | 6407 | TAACCGCGCCSTTCATTCTGT | 0.393 | — | — | — | — | — | 0.446 | 0.386 |
| 23 | 7 | 127446016 | 11981584 | 8427 | GAAGTTGCCARCCCCACATCA | 0.261 | 0.039 | — | — | — | — | 0.116 | 0.303 |
| 23 | 7 | 127451842 | 2167289 | 8428 | GTGGTATCCTKACATGCTGTA | 0.608 | 0.878 | 0.619 | — | — | — | 0.365 | 0.641 |
| 23 | 7 | 127456753 | 791595 | 8429 | GGGCAGCTGTRGCCACAGAAT | 1.522 | 1.537 | 0.728 | 0.868 | — | — | 1.143 | 1.594 |
| 23 | 7 | 127459767 | 791600 | 8430 | ATGCCAACTCRGGCCACATGC | — | — | — | — | — | — | — | — |
| 23 | 7 | 127464579 | 1376349 | 6409 | TGGTTTAAGCYTTTGAGCTCT | 2.033 | 1.803 | 1.812 | 0.415 | 0.688 | — | 1.709 | 2.125 |
| 23 | 7 | 127469340 | 4731424 | 8431 | CCCAGGGGCTKGCATTCTCGG | 1.638 | 1.273 | 1.175 | 1.213 | 0.752 | — | 1.411 | 1.725 |
| 23 | 7 | 127475802 | 2278815 | 8432 | AATGAGAGGGRCTGTGTAAGG | 0.147 | 0.538 | 0.470 | 1.698 | — | — | 0.034 | 0.155 |
| 23 | 7 | 127478340 | 4731427 | 8433 | GAGATCAGATYTTTCTGATGA | — | — | — | — | — | — | — | — |
| 23 | 7 | 127484013 | 11763517 | 6411 | GTGGCCATTAYTTGAGAGTGA | 0.683 | 0.487 | 0.325 | — | — | — | 0.392 | 0.692 |
| 23 | 7 | 127488256 | 3828942 | 8434 | TCACCTGGGTRCAGGATACAA | 0.084 | 0.501 | — | — | — | — | 0.018 | 0.106 |
| 23 | 7 | 127491020 | 11761556 | 8435 | GACAGGAGGGMAAGGGCCATG | 0.388 | — | — | — | — | — | 0.196 | 0.389 |
| 24 | 7 | 147429773 | 9648523 | 8436 | AGAAATCACAMTTTTTGCAAA | 0.033 | — | — | — | — | — | 0.023 | 0.019 |
| 24 | 7 | 147433108 | 13244714 | 8437 | AGGGTTGAAARTGCTTCTAGG | 0.421 | 1.613 | — | — | — | — | 0.740 | 0.419 |
| 24 | 7 | 147437045 | 4726947 | 6676 | TTTAGATGTASATTAAGACCC | 1.295 | 1.015 | 1.009 | — | — | — | 0.946 | 1.311 |
| 24 | 7 | 147440758 | 2373341 | 8438 | AAGAATGAACRGGAATCACAT | — | — | — | — | — | — | — | — |
| 24 | 7 | 147450906 | 11514853 | 8439 | AATCTACATARAGCAACTGGG | — | — | — | — | — | — | — | — |
| 24 | 7 | 147453963 | 4726950 | 8440 | CTGTAATTAARTACTTTTCCC | 0.614 | 0.580 | 0.953 | 0.926 | — | — | 0.564 | 0.614 |
| 24 | 7 | 147458148 | 1918298 | 8441 | CGTATTTTGTKCATGATTCTA | 0.091 | 0.603 | 0.410 | 1.651 | 1.669 | — | 0.365 | 0.107 |
| 24 | 7 | 147461850 | 1918296 | 8442 | CAGATTACACRAAGGAGTGAA | 0.180 | 0.686 | 2.503 | 0.985 | 1.230 | 1.922 | 0.049 | 0.196 |
| 24 | 7 | 147468476 | 7779793 | 8443 | AGATTCACTGYAGTCAGAAGA | 0.425 | 1.819 | 2.190 | 1.887 | 0.986 | 0.934 | 0.266 | 0.489 |
| 24 | 7 | 147473270 | 10281587 | 6678 | CTATCTCTTAYATTGCCCAGT | — | — | — | — | — | — | — | — |
| 24 | 7 | 147478836 | 11767934 | 6679 | TTGTCAGATGSTCTTCACACA | 2.329 | 1.847 | 1.343 | 1.667 | 1.114 | 0.935 | 1.776 | 2.367 |
| 24 | 7 | 147485200 | 2037869 | 6680 | TGAGGTCATGYGTAGTAAACA | 0.740 | 1.733 | 1.341 | 0.842 | 1.698 | — | 2.078 | 0.762 |
| 24 | 7 | 147491445 | — | 6681 | TTTTAGAAGTRTGCTCATTTA | — | — | — | — | — | — | — | — |
| 24 | 7 | 147497490 | 1177942 | 8444 | AAACAGTCTAYGCTTTGGCAA | 0.845 | 0.725 | 1.064 | 1.361 | — | — | 2.150 | 0.867 |
| 24 | 7 | 147502534 | 1637864 | 6682 | ACATGTCACCRAGAGAAAATG | 0.000 | 0.665 | 0.736 | — | — | — | 1.080 | 0.012 |
| 24 | 7 | 147508498 | 1730397 | 8445 | GGCTATTACCYCAATCTGAAA | 0.000 | 0.000 | — | — | — | — | 1.080 | 0.012 |
| 24 | 7 | 147514126 | 1637854 | 8446 | AAAAGAAGAARTCTCCTTTGA | 0.000 | — | — | — | — | — | 1.080 | 0.012 |
| 25 | 7 | 149524163 | 4725887 | 8447 | TGGCAGCGTGRTGTGTGGACA | 2.923 | — | — | — | — | — | 2.807 | 2.975 |
| 25 | 7 | 149556724 | 2888635 | 6721 | TGCATCGATCKGGTGAGACAC | 1.117 | 1.686 | — | — | — | — | 1.374 | 1.163 |
| 25 | 7 | 149591461 | 7810209 | 6723 | TTATAGTGGGYGATGTGTAAA | 0.498 | 0.540 | 1.212 | — | — | — | 0.214 | 0.463 |
| 25 | 7 | 149624893 | 2222524 | 6725 | ACAATGGGTGRCTTTATTTAT | — | — | — | — | — | — | — | — |
| 25 | 7 | 149649666 | 7810752 | 6726 | TGAAAATTCARCTTGTTCAAG | — | — | — | — | — | — | — | — |
| 25 | 7 | 149690467 | 4725919 | 8448 | GGATCCCACTYGTTCTTCCCC | 0.228 | 0.978 | 0.568 | 0.724 | — | — | 0.082 | 0.265 |
| 25 | 7 | 149706735 | 13242186 | 6728 | ATCTCTGAGTSCAGCAGTAAC | 1.949 | 1.195 | 0.652 | 0.507 | 0.526 | — | 2.052 | 1.980 |
| 25 | 7 | 149738096 | 4367453 | 6730 | TTAAAAAGGTYATTCCTACCC | — | — | — | — | — | — | — | — |
| 25 | 7 | 149749542 | — | 6731 | TCTGTTTTTCYCCAGCAGAAG | 0.000 | 1.125 | 0.897 | 0.791 | — | — | 0.848 | 0.006 |
| 25 | 7 | 149756449 | 10253121 | 6732 | ATGTGACAGTYTCTAGTTCCC | 0.713 | 0.457 | 1.178 | — | — | — | 1.000 | 0.732 |
| 25 | 7 | 149763839 | 10272462 | 6733 | AGGAATTTGARTGAATGGGCA | 0.734 | 1.318 | — | — | — | — | 1.000 | 0.732 |
| 25 | 7 | 149768593 | 6946579 | 6734 | TTGCCTAAGTRTTTAGAAAGT | 0.683 | — | — | — | — | — | 0.400 | 0.699 |
| 26 | 7 | 150246812 | 6951528 | 6762 | CCTGGGCTGAMCCAATGAGCA | 1.798 | — | — | — | — | — | 3.617 | 1.856 |
| 26 | 7 | 150260307 | 6464131 | 6763 | GGTGCCCGCTSCTTCCCGTTC | — | — | — | — | — | — | — | — |
| 26 | 7 | 150269456 | 6979622 | 6764 | TGTGCCCTAAYGCAGCCCTCG | — | — | — | — | — | — | — | — |
| 26 | 7 | 150286812 | 6464132 | 6765 | GCTTCGTGAARGTAGGTATTC | 1.388 | 0.812 | — | — | — | — | 0.883 | 1.380 |
| 26 | 7 | 150295919 | 4725395 | 6766 | CTTCTTTCCARTTAAAATATT | 0.817 | 0.764 | 0.577 | — | — | — | 0.534 | 0.820 |
| 26 | 7 | 150312660 | 4726005 | 6767 | ACACAGAAGTYATCACTCACA | 2.171 | 0.995 | 0.638 | 0.657 | — | — | 1.705 | 2.196 |
| 26 | 7 | 150320869 | 2257069 | 6768 | GCAATTTCCCRATGCGGGCCA | — | — | — | — | — | — | — | — |
| 26 | 7 | 150339257 | 310583 | 8449 | GTGAGCTTGARATCCTGCAGC | — | — | — | — | — | — | — | — |
| 26 | 7 | 150348102 | 2608288 | 6769 | AGTAAAGGGASAAAGTTCTTC | 0.305 | 0.847 | 0.942 | — | — | — | 0.116 | 0.319 |
| 26 | 7 | 150355235 | 7784344 | 6770 | GGCTATATTAWAAACTAATAC | 0.292 | 0.090 | — | — | — | — | 1.019 | 0.294 |
| 26 | 7 | 150373700 | 1870238 | 6772 | AATGACCACASGGCTGCTGTC | 0.419 | — | — | — | — | — | 0.198 | 0.412 |
| 28 | 9 | 26960384 | 7870787 | 8450 | ATGACTTTGGWCTGTTCCAAA | 0.378 | — | — | — | — | — | 0.166 | 0.413 |
| 28 | 9 | 26969027 | 7849248 | 8451 | CAGAGTTTTTYGTTTTCTGTA | 0.377 | 0.421 | — | — | — | — | 0.166 | 0.413 |
| 28 | 9 | 26986170 | 7018585 | 8452 | GTTTGGAAGASCAATATTTTG | 0.093 | 0.628 | 0.507 | — | — | — | 0.040 | 0.090 |
| 28 | 9 | 26996575 | 10967657 | 8453 | AGTCTGGGTGRCAAGTGGTAC | 0.381 | 0.382 | 0.483 | 1.087 | — | — | 0.173 | 0.417 |
| 28 | 9 | 27003594 | 7040505 | 8454 | GTTTTAGCTAKTTTGATAGGT | 0.011 | 0.595 | 0.939 | 1.176 | — | — | 0.075 | 0.033 |
| 28 | 9 | 27008020 | 12337896 | 6914 | AATCATTTCARAAAGGTATTA | 0.019 | 1.648 | 1.221 | — | — | — | 0.061 | 0.017 |
| 28 | 9 | 27018947 | 7390085 | 6915 | TGGAAAAGATRTCTAACCTCC | 2.317 | 1.548 | — | — | — | — | 1.828 | 2.352 |
| 28 | 9 | 27026804 | 10967677 | 8455 | GAAAAGTGGCRTGTGATTTAC | — | — | — | — | — | — | — | — |
| 28 | 9 | 27031315 | 7045747 | 6916 | ACATTCCTGTYAGAGCACTTA | 0.017 | — | — | — | — | — | 0.001 | 0.024 |
| 28 | 9 | 27037246 | 10812520 | 8456 | TCAGCAGTAAYCTCTCTTATG | — | — | — | — | — | — | — | — |
| 29 | 9 | 70416653 | 10435958 | 8457 | AAATTCCTTAWGTTCAAATGT | 0.424 | — | — | — | — | — | 0.349 | 0.449 |
| 29 | 9 | 70433985 | 7865858 | 8458 | AGCACATTTARTCTCTGAGGA | 0.762 | 0.072 | — | — | — | — | 1.065 | 0.810 |
| 29 | 9 | 70450716 | 7862783 | 8459 | GAGGCCCAATRAATGCTGGCT | — | — | — | — | — | — | — | — |
| 29 | 9 | 70470740 | 7024154 | 8460 | CTGAGTGCATMGTGAATTCCA | 0.030 | 0.207 | 0.358 | — | — | — | 0.009 | 0.036 |
| 29 | 9 | 70487522 | 4297092 | 8461 | TACTTGGAGTWAATTTATTAG | 0.061 | 1.364 | 0.713 | 0.264 | — | — | 0.037 | 0.080 |
| 29 | 9 | 70497790 | 11142532 | 6979 | ATGGAGAGAAYAGAGGAATGT | 2.303 | 1.558 | 0.588 | 0.351 | — | — | 1.695 | 2.337 |
| 29 | 9 | 70509554 | 11142536 | 6980 | TGGCTGACAGYGCCTCAGAAA | 0.790 | 1.049 | 0.668 | — | — | — | 0.431 | 0.799 |
| 29 | 9 | 70529809 | 10868873 | 8462 | ATTCCTCTCTMTGTTTCACTA | — | — | — | — | — | — | — | — |
| 29 | 9 | 70545326 | 11142556 | 8463 | AATGTTAAACRTGCTAAATAT | 0.738 | 0.170 | — | — | — | — | 0.451 | 0.765 |
| 29 | 9 | 70587221 | 11142561 | 8464 | GTAACAGTTGYCCTGCCTACC | 0.813 | — | — | — | — | — | 0.506 | 0.867 |
| 30 | 9 | 93389779 | 10119086 | 8465 | CTAAACACTAWCTCTGTTATA | 1.784 | — | — | — | — | — | 1.435 | 1.783 |
| 30 | 9 | 93410054 | 6479494 | 8466 | CCAACTTAGCRTCATTTGATG | 0.071 | 1.213 | — | — | — | — | 0.151 | 0.070 |
| 30 | 9 | 93432674 | 2150752 | 8467 | TGTGGTGGTGKTTTCTGACTC | 2.566 | 0.990 | 0.939 | — | — | — | 1.984 | 2.574 |
| 30 | 9 | 93459352 | 12551314 | 7054 | TCCCAAGAGGMAATTGTGGGG | 0.000 | 2.013 | 0.720 | 0.435 | — | — | 0.000 | 0.016 |
| 30 | 9 | 93475886 | 1806458 | 7055 | GGTGTTCCTCRCAGGTGGTGA | — | — | — | — | — | — | — | — |
| 30 | 9 | 93485419 | 6479499 | 7056 | AGCCCTGTAGWTCCTTGAAGT | 3.561 | 2.215 | 1.193 | 0.443 | 0.288 | — | 2.916 | 3.619 |
| 30 | 9 | 93491981 | — | 7057 | ACCAACACGGYACACTCACAC | — | — | — | — | — | — | — | — |
| 30 | 9 | 93498640 | 10992831 | 7058 | CTTCCGATTGRACTGTCCCTT | 2.673 | 2.610 | 1.249 | 1.060 | — | — | 2.490 | 2.723 |
| 30 | 9 | 93527276 | 7024542 | 7059 | TACCTTCCAGYGCCATCAGGA | 0.827 | 2.222 | 1.902 | — | — | — | 0.469 | 0.854 |
| 30 | 9 | 93548630 | 4744272 | 8468 | TGTCATCTGTRTTCTATGATC | 0.060 | 1.674 | — | — | — | — | 0.683 | 0.073 |
| 30 | 9 | 93563775 | 2398871 | 7061 | CTACACGAGAYAACTGACATA | 1.860 | — | — | — | — | — | 1.439 | 1.853 |
| 31 | 9 | 110502598 | 4132110 | 8469 | AAACACATATYCCCCAACTTT | — | — | — | — | — | — | — | — |
| 31 | 9 | 110521910 | 3001140 | 8470 | CTAGTAACTAYTTCAGAGAAA | 0.000 | — | — | — | — | — | 0.161 | 0.002 |
| 31 | 9 | 110542563 | 4524871 | 8471 | TCAATGAAATSTGGTGCTGGA | 0.228 | 0.246 | — | — | — | — | 0.115 | 0.199 |
| 31 | 9 | 110565516 | 4336672 | 8472 | TCCCCAAAACMCAGGCAAAAA | 1.064 | 1.026 | 0.657 | — | — | — | 0.687 | 1.098 |
| 31 | 9 | 110586553 | 4144418 | 7103 | ACAGCTAACTYAGAAAGACAG | 0.950 | 1.793 | 0.854 | 0.790 | — | — | 1.457 | 0.988 |
| 31 | 9 | 110612005 | 10980564 | 7106 | TACGAATTTCRCAGGTTTTCA | 0.970 | 1.939 | 1.094 | 0.969 | — | — | 1.181 | 0.966 |
| 31 | 9 | 110631784 | 2254097 | 8473 | CATGAGGTTGRGTAAGACACC | 1.463 | 0.682 | 0.982 | — | — | — | 3.044 | 1.520 |
| 31 | 9 | 110648651 | 1409686 | 7108 | TGCTCTGGTTMGTATTGGTAG | 1.991 | 0.943 | — | — | — | — | 1.726 | 2.021 |
| 31 | 9 | 110675982 | 4481678 | 8474 | TATAATTTCTYAAGCAGATAC | 0.080 | — | — | — | — | — | 0.101 | 0.063 |
| 31 | 9 | 110688444 | 512483 | 8475 | CCTCCTCTCASAGAAAAAGAG | — | — | — | — | — | — | — | — |
| 32 | 10 | 20313589 | 1111367 | 7179 | GGACCTCAGAYAGTATAAGTT | — | — | — | — | — | — | — | — |
| 32 | 10 | 20324955 | 7079519 | 8476 | AAGAGGATGGRGTGTCTTATA | 0.515 | — | — | — | — | — | 0.441 | 0.569 |
| 32 | 10 | 20334358 | 7913119 | 8477 | CACTAGTTGCRGAAACAAAAA | 0.838 | 0.263 | — | — | — | — | 0.470 | 0.831 |
| 32 | 10 | 20347228 | 2884508 | 8478 | TACCTGTTTTSCCCATACTAT | 0.119 | 0.335 | 0.061 | — | — | — | 0.122 | 0.132 |
| 32 | 10 | 20359198 | 7907793 | 8479 | TATACAAATGRCCATGAATTC | 0.945 | 0.112 | 0.411 | 0.269 | — | — | 1.367 | 0.966 |
| 32 | 10 | 20370658 | 7070397 | 8480 | AACAGAAATAYTTTTTCTCCC | 0.512 | 0.488 | 0.109 | 1.053 | 0.289 | — | 1.095 | 0.531 |
| 32 | 10 | 20382322 | 7923873 | 8481 | AATTGGTATGRGTTCCTACCA | 0.022 | 0.222 | 0.710 | 0.283 | — | — | 0.202 | 0.025 |
| 32 | 10 | 20395173 | 6482080 | 8482 | CCCTTTGAAGWTCTATTTGTC | 0.476 | 0.181 | 0.212 | — | — | — | 0.312 | 0.505 |
| 32 | 10 | 20406431 | 12250505 | 7189 | ATGGGGTTACRATGGGGTTAG | — | — | — | — | — | — | — | — |
| 32 | 10 | 20418225 | 10827961 | 8483 | GTATGAGTCAYGAATGTATTT | 0.231 | 0.303 | — | — | — | — | 0.046 | 0.192 |
| 32 | 10 | 20430199 | 7090580 | 8484 | AGCTCAGCCAKTATCAGAAAG | 0.282 | — | — | — | — | — | 0.643 | 0.260 |
| 33 | 10 | 100843202 | 2902259 | 8485 | ACTGTTTTGCRCTCCCATGAC | 0.568 | — | — | — | — | — | 0.352 | 0.596 |
| 33 | 10 | 100856514 | 6584235 | 8486 | TTATAGCTAAKAAAGTTGGGG | — | — | — | — | — | — | — | — |
| 33 | 10 | 100873537 | 1954295 | 8487 | AGCAGTCCTARTCACAGGCAA | 0.884 | 0.384 | — | — | — | — | 0.943 | 0.887 |
| 33 | 10 | 100889285 | 10786510 | 8488 | AATTACACTAYAAATCCCATC | 0.056 | 1.031 | 0.527 | — | — | — | 0.304 | 0.061 |
| 33 | 10 | 100898963 | 1010357 | 8489 | ACTTTTTTAASAACAGAAGCT | 0.675 | 0.544 | 0.531 | 0.430 | — | — | 0.367 | 0.690 |
| 33 | 10 | 100912517 | 10883285 | 8490 | TCCACTTTGGYTTCAAAATGT | — | — | — | — | — | — | — | — |
| 33 | 10 | 100932439 | 2796759 | 8491 | GATTAACCATYCTATCTTGGA | 0.424 | 0.348 | 0.633 | 0.438 | 0.593 | — | 0.200 | 0.416 |
| 33 | 10 | 100947920 | 4456193 | 8492 | GCAAAGGCCCRGTTTCAAATC | 0.352 | 0.156 | 0.281 | 0.712 | — | — | 0.175 | 0.349 |
| 33 | 10 | 100965767 | 10786522 | 8493 | TCTGATAATGYTGCTGATATT | 0.341 | 0.233 | 0.397 | — | — | — | 0.198 | 0.374 |
| 33 | 10 | 100978209 | 2902263 | 8494 | AAAAACATGCWTACAAATAAA | 0.389 | 0.631 | — | — | — | — | 0.224 | 0.400 |
| 33 | 10 | 100989074 | 17094130 | 7334 | GTAAGAAAACRTGAAGATTAA | 0.483 | — | — | — | — | — | 0.309 | 0.481 |
| 34 | 11 | 11861860 | 4597067 | 8495 | TCATCCACCARCTAGAGTATA | 4.597 | — | — | — | — | — | 3.888 | 4.627 |
| 34 | 11 | 11875274 | 4360694 | 8496 | GGTAACTGGTYCTGTATAGTC | — | — | — | — | — | — | — | — |
| 34 | 11 | 11887874 | 4910429 | 7378 | TAATTTCATAYAGTGCCTTTG | 4.599 | 4.472 | — | — | — | — | 3.952 | 4.644 |
| 34 | 11 | 11896387 | 12225231 | 8497 | AAGTGGCCTCYGTTCTTCAAA | — | — | — | — | — | — | — | — |
| 34 | 11 | 11911794 | 6485303 | 7379 | TGAGACATAAYTGGTGTCAAT | 0.101 | 4.496 | 3.775 | — | — | — | 0.155 | 0.116 |
| 34 | 11 | 11930555 | 16910238 | 7380 | ATAGGAGCCARTAGAGAAACA | — | — | — | — | — | — | — | — |
| 34 | 11 | 11936250 | 1979687 | 7381 | ACTTGCTATCRCACAAAAGAG | 4.444 | 4.422 | 2.992 | 2.498 | — | — | 3.771 | 4.470 |
| 34 | 11 | 11944883 | 7480000 | 7382 | AGCATCAACASGGGAAAAAAA | 0.961 | 3.361 | 2.452 | 2.456 | 1.793 | — | 0.871 | 0.954 |
| 34 | 11 | 11959842 | 7479744 | 8498 | AAAGGCCTGTYTCATATCCCT | 0.405 | 0.810 | 3.168 | 1.918 | — | — | 0.410 | 0.395 |
| 34 | 11 | 11973956 | 11601753 | 8499 | CATCAGCCGAYGTGCTCAGGT | 0.113 | 0.178 | 0.172 | — | — | — | 0.160 | 0.117 |
| 34 | 11 | 11990200 | 4757949 | 8500 | CTTTTTTCTTRTATCTGTTCT | 0.000 | 0.004 | — | — | — | — | 0.017 | 0.016 |
| 34 | 11 | 11998072 | 4500466 | 7389 | TTCCTCTATCRTTGTCATGCT | 0.141 | — | — | — | — | — | 0.143 | 0.160 |
| 35 | 11 | 19614433 | 16937060 | 7436 | TACAAAGCGGMTCTTGGTCTG | — | — | — | — | — | — | — | — |
| 35 | 11 | 19630248 | 1559667 | 7437 | CCAATTAAATRAAGCCTATTA | 1.521 | — | — | — | — | — | 1.113 | 1.570 |
| 35 | 11 | 19638525 | 16937087 | 7438 | CTGGACATTAKGGTTCATACG | — | — | — | — | — | — | — | — |
| 35 | 11 | 19647977 | 10833153 | 7439 | TCCTTCCTCCYTTGTCCTGGG | 0.627 | 0.954 | — | — | — | — | 1.009 | 0.628 |
| 35 | 11 | 19653940 | 2216997 | 7440 | TACAGCAATTKGTAATTCACG | 1.664 | 0.936 | 1.279 | — | — | — | 1.042 | 1.549 |
| 35 | 11 | 19659944 | 7113050 | 7441 | AGACCTGTGGRCACATCTGAA | 1.201 | 1.729 | 1.375 | 0.961 | — | — | 0.789 | 1.243 |
| 35 | 11 | 19663584 | 752459 | 7442 | AGGTATGGCCRGGCTGAGGAG | 3.303 | 1.588 | 1.083 | — | — | — | 2.631 | 3.323 |
| 35 | 11 | 19679410 | 1364790 | 8501 | GCAAGGAAAGYGCTCAAATGG | 2.817 | 1.779 | — | — | — | — | 2.233 | 2.878 |
| 35 | 11 | 19687211 | 890136 | 7443 | CTTATAGGCCRTTTTTATAAT | 2.678 | — | — | — | — | — | 2.452 | 2.617 |
| 35 | 11 | 19696994 | — | 7444 | CACAAATATAYACACATGGAA | — | — | — | — | — | — | — | — |
| 35 | 11 | 19707031 | 1559665 | 7445 | CAAGGAACGTRTGCAGAGAAA | — | — | — | — | — | — | — | — |
| 35 | 11 | 19721589 | 1346028 | 8502 | TCCTGGTGCTYTCTCCCTCCT | — | — | — | — | — | — | — | — |
| 35 | 11 | 19731509 | 1834323 | 8503 | CATACATTTTYTTATATTTTG | — | — | — | — | — | — | — | — |
| 36 | 11 | 95500304 | 7951852 | 7557 | TTCCATCAGCRCCAAATAATT | 0.197 | — | — | — | — | — | 0.346 | 0.223 |
| 36 | 11 | 95507337 | 7951538 | 8504 | ATATTTGAAGSCAGTCAAACT | 0.793 | 0.219 | — | — | — | — | 0.472 | 0.798 |
| 36 | 11 | 95516314 | 544552 | 8505 | AGGGATTTCCRATTTCCATAA | 0.122 | 0.615 | 0.629 | — | — | — | 0.974 | 0.130 |
| 36 | 11 | 95524215 | 515200 | 8506 | TCTTGACAGCWAGTAATCCGA | 0.653 | 1.180 | 0.666 | 0.539 | — | — | 0.949 | 0.721 |
| 36 | 11 | 95532144 | 1262181 | 8507 | TTTCCAGGCGRATTTGTTGTC | 0.631 | 1.248 | 0.751 | 0.455 | 0.230 | — | 0.364 | 0.663 |
| 36 | 11 | 95541663 | 2155001 | 7561 | GTGCTGGCTGSTGTGGGGATT | 0.188 | 0.415 | 1.077 | 0.333 | 0.203 | 0.056 | 0.434 | 0.198 |
| 36 | 11 | 95550186 | 7111187 | 8508 | GGCATGTCACMGGGAAAGAAG | 0.686 | 0.075 | 0.098 | 0.419 | 0.151 | — | 0.405 | 0.727 |
| 36 | 11 | 95558601 | 495494 | 8509 | CCTCTTTTGGKCCCTCCAGTC | 0.357 | 0.080 | 0.005 | 0.006 | — | — | 0.147 | 0.390 |
| 36 | 11 | 95567591 | 573424 | 8510 | GGAAGAAAAGRCACCACCGAA | 0.354 | 0.196 | 0.021 | — | — | — | 0.572 | 0.342 |
| 36 | 11 | 95576367 | 505879 | 8511 | GGAGCAAGCCWTTACAAAAAA | 0.509 | 0.055 | — | — | — | — | 0.623 | 0.510 |
| 36 | 11 | 95585236 | 575232 | 8512 | GGATCCAGCARAGAAGGAGCA | 0.588 | — | — | — | — | — | 0.302 | 0.602 |
| 37 | 12 | 56202309 | 1148557 | 8513 | AGATGGTCTCYCCTGGCCTAA | 0.561 | — | — | — | — | — | 0.312 | 0.603 |
| 37 | 12 | 56226338 | 1669297 | 8514 | TGACAGCGGARACAATTGGGA | 0.576 | 1.709 | — | — | — | — | 0.312 | 0.603 |
| 37 | 12 | 56254982 | 1678542 | 7613 | TGAAATGCCGSTGATAGAGAG | 1.975 | 1.178 | 0.584 | — | — | — | 1.576 | 2.014 |
| 37 | 12 | 56271471 | 11537654 | 8515 | TCCGGGCGGCYGACCCGCTGG | 0.844 | 0.725 | 0.089 | 0.408 | — | — | 0.519 | 0.889 |
| 37 | 12 | 56299442 | 2277324 | 7617 | TCCAAGCCGCRGCTCTCGCCA | 0.894 | 0.150 | 0.564 | 0.310 | 0.238 | — | 0.523 | 0.922 |
| 37 | 12 | 56307200 | 10083154 | 7618 | ATATACTTGTRCTTTGAAAAT | 0.993 | 0.299 | 0.167 | 0.555 | 0.381 | 0.414 | 0.595 | 0.994 |
| 37 | 12 | 56335586 | 1678520 | 8516 | CCTTCCTCCCSCTATTTTTAA | 1.191 | 0.551 | 0.423 | 0.164 | 0.788 | — | 0.784 | 1.237 |
| 37 | 12 | 56351715 | 2640629 | 7619 | GTGTAATAAAYAAAAGGCAAC | 1.216 | 0.716 | 0.344 | 0.182 | — | — | 0.819 | 1.276 |
| 37 | 12 | 56376989 | 7309600 | 7620 | GCATCCCTGCRAACAGCTTCT | 0.193 | 0.546 | 0.381 | — | — | — | 0.133 | 0.211 |
| 37 | 12 | 56393103 | 701006 | 7621 | AGGGCAATATRTAGGTTTGTT | 1.414 | 0.646 | — | — | — | — | 1.027 | 1.453 |
| 37 | 12 | 56430932 | 2069502 | 8517 | CACTCTCCTAYTCCCAACCAG | 0.940 | — | — | — | — | — | 0.683 | 0.987 |
| 39 | 14 | 49806475 | 2153553 | 7751 | TGAGCAATTAYCGTGTTACTC | 1.031 | — | — | — | — | — | 0.629 | 1.020 |
| 39 | 14 | 49822992 | 11849603 | 8518 | GGAAAAAAAASAAGTGACCCC | 1.140 | 0.898 | — | — | — | — | 0.747 | 1.167 |
| 39 | 14 | 49839467 | 2297995 | 7754 | CGCCACCAACRATGACTATAT | 1.169 | 0.881 | 0.261 | — | — | — | 0.777 | 1.206 |
| 39 | 14 | 49857963 | 2275592 | 7756 | CAAATGATGCYGTTTGGAAAA | 1.059 | 0.478 | 0.323 | 0.199 | — | — | 0.683 | 1.088 |
| 39 | 14 | 49868493 | 4901016 | 7757 | CATGCAGGCTRGAAACCCTGG | — | — | — | — | — | — | — | — |
| 39 | 14 | 49880775 | 1465160 | 7758 | CTGCAACTGAYATACTGCAGC | 0.817 | 0.222 | 0.303 | 0.462 | 0.263 | — | 0.446 | 0.837 |
| 39 | 14 | 49894783 | 11570816 | 8519 | CCGGATTGCTYCCAGTGAAAT | 0.215 | 0.089 | 0.350 | 0.306 | 0.397 | — | 1.043 | 0.235 |
| 39 | 14 | 49904329 | 9635166 | 7761 | GACTCAGATTKACTTTCTCTT | 0.709 | 0.982 | 0.135 | 0.463 | — | — | 0.575 | 0.717 |
| 39 | 14 | 49917611 | 6572667 | 8520 | GACCCAGGACRAGAGGGAACT | 0.650 | 0.458 | 0.498 | — | — | — | 0.773 | 0.604 |
| 39 | 14 | 49927723 | 1538904 | 8521 | ACCTGAGTTAYGGAAAGGAGT | 0.240 | 0.407 | — | — | — | — | 0.098 | 0.236 |
| 39 | 14 | 49937222 | 10149335 | 8522 | TGTGTAGGACRTTGAGAGCTG | 0.112 | — | — | — | — | — | 0.372 | 0.110 |
| 40 | 14 | 80648058 | 2888049 | 7822 | GAAGTGGTCARTTAAACTACC | 0.410 | — | — | — | — | — | 1.212 | 0.438 |
| 40 | 14 | 80661507 | 7158881 | 7823 | GTAAAGATGGYCCCTGTGGAA | 0.490 | 1.018 | — | — | — | — | 0.712 | 0.508 |
| 40 | 14 | 80668665 | 17111530 | 7824 | GTTACTTTCAYTAGACACTTA | 0.955 | 0.436 | 2.438 | — | — | — | 0.729 | 0.949 |
| 40 | 14 | 80677583 | 2300541 | 7825 | TGCCTGTATGKTAGTGTCCAT | 0.018 | 0.953 | 0.653 | 2.815 | — | — | 0.229 | 0.032 |
| 40 | 14 | 80690636 | 28441485 | 7826 | CTGTTAGGGGKAAGTTTAATG | 1.473 | 0.538 | 1.185 | 1.265 | 2.952 | — | 0.982 | 1.484 |
| 40 | 14 | 80699947 | 12372876 | 7827 | AATCAAAACAYGGCAAAGGGA | 1.456 | 1.777 | 0.818 | 0.832 | 1.696 | 2.860 | 0.981 | 1.485 |
| 40 | 14 | 80709737 | 1957546 | 7828 | AGTCACTTCAYCAAGACTGTA | 2.667 | 1.873 | 1.327 | 1.028 | 1.593 | 1.970 | 2.046 | 2.718 |
| 40 | 14 | 80713553 | 4899786 | 7829 | TGGCCACCAAKCAAGAGTTTG | 2.847 | 2.260 | 1.879 | 1.924 | 1.539 | 1.998 | 2.180 | 2.865 |
| 40 | 14 | 80727089 | 7149672 | 7830 | ATGTTACTTCRAGAAGCACTG | 2.147 | 1.951 | 2.136 | 2.643 | 2.096 | — | 1.520 | 2.161 |
| 40 | 14 | 80751275 | 6574632 | 8523 | GGAATATCAAWCAATCAAATT | 0.830 | 1.916 | 1.956 | 1.617 | — | — | 0.634 | 0.868 |
| 40 | 14 | 80774435 | 8021385 | 8524 | AAACTTCAAARGATTTAAATT | 1.236 | 1.617 | 1.196 | — | — | — | 0.790 | 1.242 |
| 40 | 14 | 80797377 | 7149917 | 8525 | GCTTCAGTCAYGGTGTGGGGC | — | — | — | — | — | — | — | — |
| 40 | 14 | 80819977 | 7148483 | 8526 | AAGTCTGTCARCACCATCTTT | 1.699 | 1.546 | — | — | — | — | 1.565 | 1.713 |
| 40 | 14 | 80833708 | 1885604 | 7840 | GCATGCTATAYGATTTAAATT | 3.552 | — | — | — | — | — | 3.054 | 3.432 |
| 42 | 16 | 73722875 | 7206259 | 7908 | AGAAGCATCAYGTTGCTAACA | 0.032 | — | — | — | — | — | 0.011 | 0.032 |
| 42 | 16 | 73735820 | 7198880 | 8527 | CATATCACCASAATTTATTTT | — | — | — | — | — | — | — | — |
| 42 | 16 | 73761944 | 12935567 | 7909 | ATATATCCAGYTGTAAATAGC | 0.633 | 0.484 | — | — | — | — | 0.277 | 0.594 |
| 42 | 16 | 73762714 | 17673793 | 7910 | CAGAGACTTTRGAGGAAAAAT | 0.270 | 1.356 | 0.374 | — | — | — | 0.183 | 0.245 |
| 42 | 16 | 73787731 | 1559362 | 7911 | TGGTTTTGCCYGTGTGACCAA | 0.370 | 0.174 | 0.881 | 0.616 | — | — | 0.695 | 0.406 |
| 42 | 16 | 73789787 | 10514392 | 7912 | TCTTGCGACTRCTCCAGACGT | 0.108 | 0.744 | 0.980 | 1.705 | 0.943 | — | 0.089 | 0.141 |
| 42 | 16 | 73812718 | 8051363 | 7913 | TTCTGCTTCCRATGTGCCATT | 0.759 | 0.490 | 1.465 | 1.170 | — | — | 0.409 | 0.743 |
| 42 | 16 | 73823220 | 8062565 | 7914 | TCTGAAAACAMGGCGATCCCA | — | — | — | — | — | — | — | — |
| 42 | 16 | 73837879 | 28439846 | 7915 | GGTCAGAGACRGCCTCCCCAA | 2.033 | 1.989 | 0.829 | — | — | — | 1.306 | 1.941 |
| 42 | 16 | 73851712 | 4261573 | 7916 | TTTGATTCGAYTGATGGCTAC | 0.029 | 0.835 | — | — | — | — | 0.882 | 0.045 |
| 42 | 16 | 73878532 | 4243112 | 7918 | CTGAGCAACCYTTCAAAGGTG | 0.613 | — | — | — | — | — | 0.331 | 0.638 |
| 42 | 16 | 73890482 | 11149814 | 7920 | CACCAAGAAAYCTGTGTAATG | — | — | — | — | — | — | — | — |
| 43 | 16 | 76574323 | 402904 | 7956 | GCCATTTGGGRTATAGGAAAC | 1.514 | — | — | — | — | — | 1.289 | 1.542 |
| 43 | 16 | 76594303 | 8058458 | 7959 | CAATCCTATCKGTGAATTTCT | 1.035 | 0.815 | — | — | — | — | 0.689 | 1.037 |
| 43 | 16 | 76612446 | 11643023 | 7962 | CCACTGCCCTYCATTTGCACC | 0.410 | 0.690 | 1.089 | — | — | — | 0.248 | 0.398 |
| 43 | 16 | 76634316 | 4888726 | 7963 | ATTGGGGCCAYCCTGTGCATT | 0.646 | 0.695 | 1.265 | 0.525 | — | — | 1.037 | 0.635 |
| 43 | 16 | 76660674 | 6564507 | 7965 | CTTGTCATTCRATCTTGTCTA | 1.256 | 0.875 | 0.894 | 0.646 | — | — | 0.904 | 1.278 |
| 43 | 16 | 76673175 | 16947096 | 7966 | TCAGGAACGAYGATTTATAAC | 0.523 | 0.251 | 0.124 | — | — | — | 0.347 | 0.537 |
| 43 | 16 | 76680185 | — | 7967 | GGGCCTATGCRTAGAGTCAGT | — | — | — | — | — | — | — | — |
| 43 | 16 | 76685815 | 1073111 | 7968 | CTCCTTGCTARTAATCAATGT | 0.386 | 0.038 | — | — | — | — | 0.368 | 0.414 |
| 43 | 16 | 76698607 | 8045088 | 7969 | CCCTTCGAATRCCTCTTGTAG | — | — | — | — | — | — | — | — |
| 43 | 16 | 76717963 | 8057015 | 8528 | CATGTGCTATMATTTAATTTT | 0.111 | — | — | — | — | — | 0.146 | 0.142 |
| 44 | 18 | 22743410 | 162632 | 8147 | TTAATTCTGAYACAGGTCGTC | 0.887 | — | — | — | — | — | 0.684 | 0.902 |
| 44 | 18 | 22768586 | 163057 | 8529 | AACCCGAAGAKCCTGGAGAGA | 2.039 | 0.584 | — | — | — | — | 1.828 | 2.089 |
| 44 | 18 | 22793630 | 12326470 | 8530 | AAATGGTTGCRCTGAACTTAA | 0.255 | 0.835 | 0.233 | — | — | — | 0.087 | 0.274 |
| 44 | 18 | 22821237 | 1175739 | 8531 | CTCTTTGCACRTGTCTGCTTC | 0.000 | 0.130 | 0.655 | 1.250 | — | — | 0.462 | 0.013 |
| 44 | 18 | 22845245 | 1185795 | 8532 | TCTTGGTGAAKGCAGGAATTA | 0.039 | 0.075 | 2.500 | 1.443 | 1.174 | — | 0.336 | 0.042 |
| 44 | 18 | 22868217 | 1941114 | 8533 | TCTCTGTTTTYGTATCCTTCC | 0.092 | 3.554 | 1.977 | 1.771 | 1.436 | 1.068 | 0.367 | 0.107 |
| 44 | 18 | 22898135 | 6508467 | 8167 | AAACCCTGGGMAAGTGATAAT | 4.804 | 3.408 | 2.199 | 1.858 | 1.305 | — | 4.259 | 4.807 |
| 44 | 18 | 22917032 | 1385753 | 8534 | TGTGAAGAGAYAGCCGTAAGG | 0.332 | 3.064 | 2.727 | 1.660 | — | — | 0.343 | 0.387 |
| 44 | 18 | 22940785 | 4800789 | 8535 | TAATCTACCTRCTTACTACCC | 0.087 | 0.238 | 2.271 | — | — | — | 0.185 | 0.103 |
| 44 | 18 | 22964271 | 2114471 | 8536 | TGACCTAGTARTTATTTTAAT | 0.187 | 0.095 | — | — | — | — | 0.195 | 0.222 |
| 44 | 18 | 22981745 | 4800791 | 8537 | TCCTCTGTAARCCCCTACAGT | 0.430 | — | — | — | — | — | 0.216 | 0.470 |
| 45 | 18 | 61556288 | 7226378 | 8227 | AAGCCCTTAARGAAATATAGA | 0.454 | — | — | — | — | — | 2.031 | 0.478 |
| 45 | 18 | 61577193 | 12963820 | 8538 | AATGAGTTGCWCAATTATCAA | 0.770 | 0.814 | — | — | — | — | 2.550 | 0.772 |
| 45 | 18 | 61595696 | 1484725 | 8231 | CTCCAAACGCRAAGCAAATTT | 1.106 | 1.803 | 0.806 | — | — | — | 2.741 | 1.114 |
| 45 | 18 | 61621115 | 4334398 | 8235 | TCGGTGAACAYGAAGCTCTAA | 0.867 | 1.883 | 1.139 | 0.598 | — | — | 0.548 | 0.875 |
| 45 | 18 | 61627292 | 11659738 | 8236 | TTGAAGTCCCRTGCATGTATT | 0.895 | 1.364 | 0.641 | 0.858 | 0.972 | — | 0.538 | 0.911 |
| 45 | 18 | 61634277 | 4144764 | 8237 | CCTATGGAGAYAAAGCCATAC | — | — | — | — | — | — | — | — |
| 45 | 18 | 61635487 | 6566179 | 8238 | TAAGAGGTATYGTCATAACAA | — | — | — | — | — | — | — | — |
| 45 | 18 | 61655933 | 9963814 | 8539 | AAACATAAGGYCTTCTTGGTC | 1.401 | 1.271 | 1.106 | 0.898 | — | — | 0.889 | 1.418 |
| 45 | 18 | 61672858 | 1484711 | 8239 | AGTTCAAGGCYTTTTAGCTTT | 2.119 | 1.231 | 0.841 | — | — | — | 1.603 | 2.175 |
| 45 | 18 | 61686997 | 2587410 | 8241 | GGAAACTTTTYTTCTATATTA | 2.107 | 1.930 | — | — | — | — | 1.603 | 2.175 |
| 45 | 18 | 61704351 | 1843184 | 8540 | TTATAGCTTCRTGATTAAAAT | 0.684 | — | — | — | — | — | 0.443 | 0.659 |
| 45 | 18 | 61717080 | 7407658 | 8541 | ACAAGGAAAAKATCTTTAAAC | — | — | — | — | — | — | — | — |
| 47 | X | 80276252 | 1538351 | 8542 | GTGACAGAGASAGAACATGAA | 0.129 | — | — | — | — | — | — | — |
| 47 | X | 80303261 | 12848561 | 8543 | CTGTCACTCARTGAAGCTCAT | 0.087 | 1.088 | — | — | — | — | — | — |
| 47 | X | 80325649 | 5959088 | 8336 | CTGTGTGCATRCTGCCTGTGT | 0.302 | 0.624 | 0.986 | — | — | — | — | — |
| 47 | X | 80348471 | 4826212 | 8338 | CTGTTTGATTRGATGACTCAT | 1.139 | 0.582 | 0.425 | 0.578 | — | — | — | — |
| 47 | X | 80387275 | 5959099 | 8340 | TTTTAATCTARCTCTTTATTC | 1.115 | 0.602 | 0.115 | 0.707 | 0.977 | — | — | — |
| 47 | X | 80411297 | 5912496 | 8342 | AAGGATGGATRTAACGGAGAG | 0.879 | 0.455 | 0.602 | 0.582 | 0.674 | — | — | — |
| 47 | X | 80435314 | 6616745 | 8345 | TTGAAGGACAYAGATGGATAC | 0.989 | 0.670 | 0.644 | 0.636 | — | — | — | — |
| 47 | X | 80466657 | — | 8348 | GCTGTTATTTWTTTCCCTAGC | — | — | — | — | — | — | — | — |
| 47 | X | 80492353 | 2602598 | 8350 | GTAGTGTGCTRGATAACAGAG | 1.787 | 1.441 | 0.709 | — | — | — | — | — |
| 47 | X | 80525324 | 1597965 | 8544 | CAAGAGTGATRTTAGCTATGT | 1.787 | 1.437 | — | — | — | — | — | — |
| 47 | X | 80552137 | 5913497 | 8545 | CACACTTATTRTCAGTCTCAC | 1.713 | — | — | — | — | — | — | — |

TABLE 4
List of longevity candidate genes from the regions identified from the genome wide and fine mapping
association analysis. The first column corresponds to the region identifier provided in Table 1.
The second and third columns correspond to the chromosome and cytogenetic band, respectively.
The fourth and fifth columns corresponds to the chromosomal start coordinates of the NCBI genome
assembly derived from build 35 (B35) and the end coordinates (the start and end position relate to
the + orientation of the NCBI assembly and don't necessarily correspond to the orientation of the gene).
The sixth and seventh columns correspond to the official gene symbol and gene name, respectively, and were
obtained from the NCBI Entre2 Gene database. The eighth column corresponds to the NCBI
Entrez Gene Identifier (Gene ID). The ninth and tenth columns correspond to the
Sequence IDs from nucleotide (cDNA) and protein entries in the Sequence Listing.

| | | | Start | End | | | | | |
|---|---|---|---|---|---|---|---|---|---|
| | | | Position | position | | | Entrez | | |
| Region ID | Chromosome | Cytogenetic Band | B35 | B35 | Gene Symbol | Gene Name | Gene ID | Nucleotide Seq ID | Protein Seq ID |
| 1 | 1 | 1q25.2 | 172931977 | 172932623 | LOC391140 | similar to ribosomal protein L13 | 391140 | — | — |
| 1 | 1 | 1q23-q25 | 173163964 | 173543629 | PAPPA2 | pappalysin 2 | 60676 | 1, 3 | 2, 4 |
| 1 | 1 | 1q25.2 | 173561861 | 173865681 | ASTN | astrotactin | 460 | 5, 7 | 6, 8 |
| 2 | 1 | 1q42-q43 | 225713614 | 225747253 | RAB4A | RAB4A, member RAS oncogene | 5867 | 9 | 10 |
| | | | | | | family | | | |
| 2 | 1 | 1q42.11-q42.3 | 225747016 | 225747983 | SPHAR | S-phase response (cyclin- | 10638 | 11 | 12 |
| | | | | | | related) | | | |
| 2 | 1 | 1q42.13 | 225766950 | 225784999 | C1orf96 | chromosome 1 open rending | 126731 | 13 | 14 |
| | | | | | | frame 96 | | | |
| 2 | 1 | 1q42.13-q42.2 | 225873730 | 225876578 | ACTA1 | actin alpha 1, skeletal muscle | 58 | 15 | 16 |
| 2 | 1 | 1q42.13 | 225883779 | 225950823 | NUP133 | nucleoporin 133 kDa | 55746 | 17 | 18 |
| 2 | 1 | 1q42 | 225959065 | 226001177 | ABCB10 | ATP-binding cassette, sub-family | 23456 | 19 | 20 |
| | | | | | | B (MDR/TAP), member 10 | | | |
| 3 | 1 | 1q42.2 | 227084993 | 227136463 | COG2 | component of oligomeric golgi | 22796 | 21 | 22 |
| | | | | | | complex 2 | | | |
| 3 | 1 | 1q42-q43 | 227145020 | 227156602 | AGT | angiotensinogen (serpin | 183 | 23 | 24 |
| | | | | | | peptidase inhibitor, clade A, | | | |
| | | | | | | member 8) | | | |
| 3 | 1 | 1q42.11-q42.3 | 227189865 | 227244243 | CAPN9 | calpain 9 | 10753 | 25, 27 | 26, 28 |
| 3 | 1 | 1q42.13-q43 | 227279601 | 227311012 | C1orf198 | chromosome 1 open reading | 84886 | 29 | 30 |
| | | | | | | frame 198 | | | |
| 3 | 1 | 1q42.2 | 227348725 | 227421319 | TTC13 | tetratricopeptide repeat domain | 79573 | 31 | 32 |
| | | | | | | 13 | | | |
| 3 | 1 | 1q42.2 | 227421558 | 227443214 | ARV1 | ARV1 homolog (yeast) | 64801 | 33 | 34 |
| 3 | 1 | 1q42.2 | 227461670 | 227482720 | FAM89A | family with sequence similarity | 375061 | 35 | 36 |
| | | | | | | 89, member A | | | |
| 3 | 1 | 1q42.2 | 227483511 | 227605268 | LOC441924 | similar to peptidylprolyl | 441924 | — | — |
| | | | | | | isomerase A isoform 1 | | | |
| 4 | 1 | 1q41-q44 | 236396793 | 236398565 | CHRM3 | cholinergic receptor, muscarinic 3 | 1131 | 37 | 38 |
| 4 | 1 | 1q43 | 236460221 | 236463588 | LOC128136 | similar to Hydroxymethylglutaryl- | 128136 | — | — |
| | | | | | | CoA synthase, cytoplasmic | | | |
| | | | | | | (HMG-CoA synthase) (3-hydroxy- | | | |
| | | | | | | 3-methylglutaryl coenzyme A | | | |
| | | | | | | synthase) | | | |
| 4 | 1 | 1q43 | 236502010 | 236964518 | FMN2 | formin 2 | 56776 | 39 | 40 |
| 4 | 1 | 1q43 | 236631871 | 236634716 | LOC266783 | proteasome 26S non-ATPase | 266783 | — | — |
| | | | | | | subunit 2 pseudogene | | | |
| 5 | 2 | 2p24.1 | 20806446 | 20922853 | FLJ21820 | hypothetical protein FLJ21820 | 60526 | 41 | 42 |
| 5 | 2 | 2p24-p23 | 21135953 | 21178597 | APOB | apolipoprotein B (including Ag(x) | 338 | 43 | 44 |
| | | | | | | antigen) | | | |
| 6 | 2 | 2p16.3 | 50017421 | 50018157 | LOC130728 | similar to 60S ribosomal protein | 130728 | — | — |
| | | | | | | L7 | | | |
| 6 | 2 | 2p16.3 | 50059139 | 51167254 | NRXN1 | neurexin 1 | 9378 | 45, 47 | 46, 48 |
| 7 | 2 | 2p16.3 | 50059139 | 51167254 | NRXN1 | neurexin 1 | 9378 | 45, 47 | 46, 48 |
| 8 | 2 | 2q14 | 121694764 | 121759005 | TFCP2L1 | transcription factor CP2-like 1 | 29842 | 49 | 50 |
| 8 | 2 | 2q14.2-q14.3 | 121811585 | 122123282 | CLASP1 | cytoplasmic linker associated | 23332 | 51 | 52 |
| | | | | | | protein 1 | | | |
| 8 | 2 | 2q14.3 | 122182315 | 122186434 | LOC440902 | similar to Nucleophosmin (NPM) | 440902 | 53 | 54 |
| | | | | | | (Nucleolar phosphoprotein B23) | | | |
| | | | | | | (Numatrin) (Nucleolar protein | | | |
| | | | | | | NO38) | | | |
| 8 | 2 | 2q14.3 | 122200784 | 122210698 | MKI67IP | MKI67 (FHA domain) interacting | 84365 | 55 | 56 |
| | | | | | | nucleolar phosphoprotein | | | |
| 8 | 2 | 2q21.1 | 122229351 | 122241659 | TSN | translin | 7247 | 57 | 58 |
| 9 | 2 | 2q14 | 127521837 | 127581094 | BIN1 | bridging integrator 1 | 274 | 59, 61, 63, 65, 67, 69, 71, 73, | 60, 62, 64, 66, 68, 70, |
| | | | | | | | | 75, 77 | 72, 74, 76, 78 |
| 9 | 2 | 2q14.3 | 127657638 | 127700162 | FLJ16008 | FLJ16008 protein | 339761 | 79 | 80 |
| 9 | 2 | 2q21 | 127731096 | 127767982 | ERCC3 | excision repair cross- | 2071 | 81 | 82 |
| | | | | | | complementing rodent repair | | | |
| | | | | | | deficiency, complementation | | | |
| | | | | | | group 3 (xeroderma | | | |
| | | | | | | pigmentosum group B | | | |
| | | | | | | complementing) | | | |
| 9 | 2 | 2q14.3 | 127780008 | 127817035 | MAP3K2 | mitogen-activated protein kinase | 10746 | 83 | 84 |
| | | | | | | kinase kinase 2 | | | |
| 9 | 2 | 2q13-q14 | 127892247 | 127903048 | PROC | protein C (inactivator of | 5624 | 85 | 86 |
| | | | | | | coagulation factors Va and VIIIa) | | | |
| 9 | 2 | 2q14.3 | 127954620 | 128000274 | IWS1 | hypothetical protein LOC55677 | 55677 | 87 | 88 |
| 10 | 2 | 2q24.2 | 161990447 | 162093433 | PSMD14 | proteasome (prosome, | 10213 | 89 | 90 |
| | | | | | | macropain) 26S subunit, non- | | | |
| | | | | | | ATPase, 14 | | | |
| 10 | 2 | 2q24 | 162098127 | 162107080 | TBR1 | T-box, brain, 1 | 10716 | 91 | 92 |
| 10 | 2 | 2q24.2 | 162182642 | 162189817 | AHCTF1P | AT hook containing transcription | 285118 | — | — |
| | | | | | | factor 1 pseudogene | | | |
| 10 | 2 | 2q24.2 | 162262089 | 162263367 | LOC391458 | similar to keratin complex 1, | 391458 | — | — |
| | | | | | | acidic, gene 18 | | | |
| 10 | 2 | 2q23-q24 | 162306453 | 162665750 | SLC4A10 | solute carrier family 4, sodium | 57282 | 93 | 94 |
| | | | | | | bicarbonate transporter-like, | | | |
| | | | | | | member 10 | | | |
| 10 | 2 | 2q24.3 | 162674264 | 162756559 | DPP4 | dipeptidyl-peptidase 4 (CD26, | 1803 | 95 | 96 |
| | | | | | | adenosine deaminase | | | |
| | | | | | | complexing protein 2) | | | |
| 10 | 2 | 2q24.2 | 162759374 | 162759688 | DDPP | deafness dystonia pseudogene | 399520 | — | — |
| 10 | 2 | 2q36-q37 | 162824895 | 162834264 | GCG | glucagon | 2641 | 97 | 98 |
| 10 | 2 | 2q23 | 162852709 | 162925552 | FAP | fibroblast activation protein, | 2191 | 99 | 100 |
| | | | | | | alpha | | | |
| 10 | 2 | 2p24.3-q24.3 | 162949097 | 163000546 | IFIH1 | interferon induced with helicase | 64135 | 101 | 102 |
| | | | | | | C domain 1 | | | |
| 11 | 2 | 2q33 | 198773985 | 198838554 | PLCL1 | phospholipase C-like 1 | 5334 | 103 | 104 |
| 12 | 3 | 3p25.3 | 6789724 | 6790033 | MRPS36P1 | mitochondrial ribosomal protein | 347705 | — | — |
| | | | | | | S36 pseudogene 1 | | | |
| 12 | 3 | 3p26.1-p25.1 | 6877927 | 7758217 | GRM7 | glutamate receptor, metabotropic 7 | 2917 | 105, 107, 109 | 106, 108, 110 |
| 13 | 3 | 3p22.3 | 32701702 | 32790358 | CNOT10 | CCR4-NOT transcription | 25904 | 111 | 112 |
| | | | | | | complex, subunit 10 | | | |
| 13 | 3 | 3p22.3 | 32799068 | 32806379 | LOC389101 | similar to 60S ribosomal protein | 389101 | — | — |
| | | | | | | L23a | | | |
| 13 | 3 | 3p22.3 | 32832845 | 32833862 | UNQ6490 | similar to YPLR6490 | 389102 | 113 | — |
| 13 | 3 | 3p22.3 | 32834892 | 32908307 | LIN41 | abnormal cell LINeage LIN-41 | 131405 | 114 | 115 |
| 13 | 3 | 3p24 | 32968056 | 32971404 | CCR4 | chemokine (C-C motif) receptor 4 | 1233 | 116 | 117 |
| 13 | 3 | 3p21.33 | 33013236 | 33113635 | GLB1 | galactosidase, beta 1 | 2720 | 118 | 119 |
| 13 | 3 | 3p22.3 | 33130571 | 33160004 | CRTAP | cartilage associated protein | 10491 | 120 | 121 |
| 13 | 3 | 3p22.3 | 33166541 | 33246551 | SUSD5 | sushi domain containing 5 | 26032 | 122 | 123 |
| 13 | 3 | 3p22.3 | 33293938 | 33403129 | FBXL2 | F-box and leucine-rich repeat | 25827 | 124 | 125 |
| | | | | | | protein 2 | | | |
| 14 | 3 | 3p22.1 | 39826307 | 40276816 | MYRIP | myosin VIIA and Rab interacting | 25924 | 126 | 127 |
| | | | | | | protein | | | |
| 14 | 3 | 3p22.1 | 40326194 | 40328902 | EIF1B | eukaryotic translation initiation | 10289 | 128 | 129 |
| | | | | | | factor 1B | | | |
| 14 | 3 | 3p21.3 | 40403694 | 40445114 | ENTPD3 | ectonucleoside triphosphate | 956 | 130 | 131 |
| | | | | | | diphosphohydrolase 3 | | | |
| 14 | 3 | 3p22-p21.2 | 40473819 | 40478863 | RPL14 | ribosomal protein L14 | 9045 | 132, 134 | 133, 135 |
| 14 | 3 | 3p22.1 | 40493641 | 40504881 | ZNF619 | zinc finger protein 619 | 285267 | 136 | 137 |
| 14 | 3 | 3p22.1 | 40522487 | 40534204 | ZNF620 | zinc finger protein 620 | 253639 | 138 | 139 |
| 14 | 3 | 3p22.1 | 40541373 | 40550438 | ZNF621 | zinc finger protein 621 | 285268 | 140 | 141 |
| 14 | 3 | 3p22.1 | 40612702 | 40632425 | LOC442079 | similar to Elongation factor 1- | 442079 | — | — |
| | | | | | | gamma (EF-1-gamma) (eEF-1B | | | |
| | | | | | | gamma) | | | |
| 15 | 4 | 4q21.1 | 77007008 | 77092543 | VDP | vesicle docking protein p115 | 8615 | 142 | 143 |
| 15 | 4 | 4q21.1 | 77138207 | 77180860 | PPEF2 | protein phosphatase, EF-hand | 5470 | 144, 146, 148 | 145, 147, 149 |
| | | | | | | calcium binding domain 2 | | | |
| 15 | 4 | 4q21.1 | 77195804 | 77219333 | ASAHL | N-acylsphingosine | 27163 | 150 | 151 |
| | | | | | | amidohydrolase (acid | | | |
| | | | | | | ceramidase)-like | | | |
| 15 | 4 | 4q21.1 | 77228248 | 77260314 | SDAD1 | SDA1 domain containing 1 | 55153 | 152 | 153 |
| 15 | 4 | 4q21 | 77279802 | 77285820 | CXCL9 | chemokine (C—X—C motif) ligand 9 | 4283 | 154 | 155 |
| 15 | 4 | 4q21 | 77299452 | 77301829 | CXCL10 | chemokine (C—X—C motif) ligand | 3627 | 156 | 157 |
| | | | | | | 10 | | | |
| 15 | 4 | 4q21.2 | 77312021 | 77314412 | CXCL11 | chemokine (C—X—C motif) ligand | 6373 | 158 | 159 |
| | | | | | | 11 | | | |
| 15 | 4 | 4p15.1-p14\|4p15.1-p14\|4p15.1-p14 | 77353042 | 77391120 | ART3 | ADP-ribosyltransferase 3 | 419 | 160 | 161 |
| 15 | 4 | 4q21.1 | 77392997 | 77426834 | NUP54 | nucleoporin 54 kDa | 53371 | 162 | 163 |
| 15 | 4 | 4q21.1 | 77437073 | 77492214 | SCARB2 | scavenger receptor class B, | 950 | 164 | 165 |
| | | | | | | member 2 | | | |
| 16 | 4 | 4q24 | 100853159 | 100901804 | MTTP | microsomal triglyceride transfer | 4547 | 166 | 167 |
| | | | | | | protein | | | |
| 16 | 4 | 4q23 | 100914872 | 100933921 | LOC285556 | hypothetical protein LOC285556 | 285556 | 168 | 169 |
| 16 | 4 | 4q25-q27 | 101095168 | 101148489 | DAPP1 | dual adaptor of phosphotyrosine | 27071 | 170 | 171 |
| | | | | | | and 3-phosphoinositides | | | |
| 16 | 4 | 4q23 | 101159543 | 101172881 | MAP2K1IP1 | mitogen-activated protein kinase | 8649 | 172 | 173 |
| | | | | | | kinase 1 interacting protein 1 | | | |
| 16 | 4 | 4q23 | 101177747 | 101205426 | DNAJB14 | DnaJ (Hsp40) homolog, | 79982 | 174, 176 | 175, 177 |
| | | | | | | subfamily B, member 14 | | | |
| 16 | 4 | 4q24 | 101226421 | 101228611 | H2AFZ | H2A histone family, member Z | 3015 | 178 | 179 |
| 17 | 4 | 4q28.1 | 126595172 | 126600361 | LOC339951 | similar to Cadherin-related tumor | 339951 | 180 | 181 |
| | | | | | | suppressor precursor (Fat | | | |
| | | | | | | protein) | | | |
| 17 | 4 | 4q28.1 | 126672696 | 126770528 | FAT4 | FAT tumor suppressor homolog | 79633 | 182 | 183 |
| | | | | | | 4 (Drosophila) | | | |
| 18 | 4 | 4q31.21 | 143307499 | 143710137 | INPP4B | inositol polyphosphate-4- | 8821 | 184 | 185 |
| | | | | | | phosphatase, type II, 105 kDa | | | |
| 19 | 6 | 6p22.1 | 27208074 | 27208554 | HIST1H2BJ | histone 1, H2bj | 8970 | 186 | 187 |
| 19 | 6 | 6p22.1 | 27208800 | 27211050 | HIST1H2AG | histone 1, H2ag | 8969 | 188 | 189 |
| 19 | 6 | 6p21.33 | 27214052 | 27222598 | HIST1H2BK | histone 1, H2bk | 85236 | 190 | 191 |
| 19 | 6 | 6p21.33 | 27215067 | 27215436 | HIST1H4I | histone 1, H4i | 8294 | 192 | 193 |
| 19 | 6 | 6p21.33 | 27222887 | 27223373 | HIST1H2AH | histone 1, H2ah | 85235 | 194 | 195 |
| 19 | 6 | 6p22.1 | 27287002 | 27287670 | LOC442171 | similar to ribosomal protein L10 | 442171 | — | — |
| 19 | 6 | 6p21 | 27323487 | 27332229 | PRSS16 | protease, serine, 16 (thymus) | 10279 | 196 | 197 |
| 19 | 6 | 6p22.1 | 27343934 | 27344990 | LOC442172 | similar to cell division cycle | 442172 | — | — |
| | | | | | | associated 7 | | | |
| 19 | 6 | 6p22.1 | 27361661 | 27388776 | LOC441135 | similar to Nuclear envelope pore | 441135 | 198 | 199 |
| | | | | | | membrane protein POM 121 | | | |
| | | | | | | (Pore membrane protein of 121 kDa) | | | |
| | | | | | | (P145) | | | |
| 19 | 6 | 6p22.1 | 27400557 | 27401721 | FKSG83 | FKSG83 | 83954 | 200 | 201 |
| 19 | 6 | 6p21.3 | 27433582 | 27447283 | ZNF204 | zinc finger protein 204 | 7754 | 202 | — |
| 20 | 6 | 6q25.1 | 152220800 | 152516520 | ESR1 | estrogen receptor 1 | 2099 | 203 | 204 |
| 20 | 6 | 6q25 | 152534937 | 153050648 | SYNE1 | spectrin repeat containing, | 23345 | 205, 207, 209, 211 | 206, 208, 210, 212 |
| | | | | | | nuclear envelope 1 | | | |
| 20 | 6 | 6q25 | 152959863 | 152960263 | NANOGP11 | NANOG homeobox pseudogene | 414135 | — | — |
| | | | | | | 11 | | | |
| 20 | 6 | 6q25.2 | 153111152 | 153136877 | MYCT1 | myc target 1 | 80177 | 213 | 214 |
| 20 | 6 | 6q25 | 153164047 | 153173014 | VIP | vasoactive intestinal peptide | 7432 | 215, 217 | 216, 218 |
| 21 | 7 | 7p15.1 | 30370952 | 30385944 | LOC401320 | hypothetical LOC401320 | 401320 | 219 | 220 |
| 21 | 7 | 7p15 | 30407421 | 30446884 | GARS | glycyl-tRNA synthetase | 2617 | 221 | 222 |
| 21 | 7 | 7p15.1 | 30465440 | 30495136 | CRHR2 | corticotropin releasing hormone | 1395 | 223 | 224 |
| | | | | | | receptor 2 | | | |
| 21 | 7 | 7p15.3-p15.2 | 30564991 | 30570462 | INMT | indolethylamino N- | 11185 | 225 | 226 |
| | | | | | | methyltransferase | | | |
| 21 | 7 | 7p15.1 | 30584280 | 30705242 | FLJ22374 | hypothetical protein FLJ22374 | 84182 | 227 | 228 |
| 21 | 7 | 7p14 | 30724708 | 30738371 | AQP1 | aquaporin 1 (Colton blood group) | 358 | 229 | 230 |
| 21 | 7 | 7p14 | 30776876 | 30792383 | GHRHR | growth hormone releasing | 2692 | 231, 233 | 232, 234 |
| | | | | | | hormone receptor | | | |
| 21 | 7 | 7p14 | 30865382 | 30919551 | ADCYAP1R1 | adenylate cyclase activating | 117 | 235 | 236 |
| | | | | | | polypeptide 1 (pituitary) receptor | | | |
| | | | | | | type I | | | |
| 22 | 7 | 7p14.3 | 32681024 | 32704612 | KBTBD2 | Kelch repeat and BTB (POZ) | 25948 | 237 | 238 |
| | | | | | | domain containing 2 | | | |
| 22 | 7 | 7p14.3 | 32730196 | 32755873 | LOC441212 | PNAS-13 | 441212 | 239 | 240 |
| 22 | 7 | 7p11.1 | 32770292 | 32819782 | FKBP9 | FK506 binding protein 9, 63 kDa | 11328 | 241 | 242 |
| 22 | 7 | 7p14.3 | 32826991 | 32853758 | NT5C3 | 5-nucleotidase, cytosolic III | 51251 | 243, 245, 247 | 244, 246, 248 |
| 22 | 7 | 7p14.3 | 32907652 | 32922242 | RP9 | retinitis pigmentosa 9 (autosomal | 6100 | 249 | 250 |
| | | | | | | dominant) | | | |
| 22 | 7 | 7p14 | 32942414 | 33418920 | PTHB1 | parathyroid hormone-responsive | 27241 | 251, 253, 255, 257 | 252, 254, 256, 258 |
| | | | | | | B1 | | | |
| 23 | 7 | 7q31.3 | 126886185 | 127326610 | SND1 | staphylococcal nuclease domain | 27044 | 259 | 260 |
| | | | | | | containing 1 | | | |
| 23 | 7 | 7q31.3 | 127261077 | 127264953 | LRRC4 | leucine rich repeat containing 4 | 64101 | 261 | 262 |
| 23 | 7 | 7q31.3 | 127485995 | 127491632 | LEP | leptin (obesity homolog, mouse) | 3952 | 263 | 264 |
| 23 | 7 | 7q32.1 | 127544388 | 127577913 | RBM28 | RNA binding motif protein 28 | 55131 | 265 | 266 |
| 23 | 7 | 7q32.1 | 127584861 | 127595686 | LOC401399 | hypothetical gene supported by | 401399 | 267, 269 | 268, 270 |
| | | | | | | BC063892 | | | |
| 23 | 7 | 7q31.3-q32 | 127626283 | 127644257 | IMPDH1 | IMP (inosine monophosphate) | 3614 | 271, 273 | 272, 274 |
| | | | | | | dehydrogenase 1 | | | |
| 23 | 7 | 7q32.1 | 127689896 | 127692421 | HIG2 | hypoxia-inducible protein 2 | 29923 | 275 | 276 |
| 23 | 7 | 7q32.1 | 127710769 | 127736536 | METTL2B | methyltransferase like 2B | 55798 | 277 | 278 |
| 24 | 7 | 7q35-q36 | 145251477 | 147555734 | CNTNAP2 | contactin associated protein-like 2 | 26047 | 279 | 280 |
| 24 | 7 | 7q36.1 | 147569287 | 147580921 | LOC392145 | similar to Mtr3 (mRNA transport | 392145 | 281 | 282 |
| | | | | | | regulator 3)-homolog | | | |
| 24 | 7 | 7q36.1 | 147725305 | 147750600 | C7orf33 | chromosome 7 open reading | 202865 | 283 | 284 |
| | | | | | | frame 33 | | | |
| 25 | 7 | 7q31-q35 | 149458253 | 149472887 | LRRC61 | leucine rich repeat containing 61 | 65999 | 285 | 286 |
| 25 | 7 | 7q36.1 | 149465049 | 149467458 | C7orf29 | chromosome 7 open reading | 113763 | 287 | 288 |
| | | | | | | frame 29 | | | |
| 25 | 7 | 7q36.1 | 149473067 | 149476354 | RARRES2 | retinoic acid receptor responder | 5919 | 289 | 290 |
| | | | | | | (tazarotene induced) 2 | | | |
| 25 | 7 | 7q36.1 | 149505509 | 149508776 | REPIN1 | replication initiator 1 | 29803 | 291, 293 | 292, 294 |
| 25 | 7 | 7q36.1 | 149514098 | 149533366 | MGC33584 | hypothetical protein MGC33584 | 285971 | 295 | 296 |
| 25 | 7 | 7q36.1 | 149585772 | 149614129 | GIMAP8 | GTPase, IMAP family member 8 | 155038 | 297 | 298 |
| 25 | 7 | 7q36.1 | 149649611 | 149655809 | GIMAP7 | GTPase, IMAP family member 7 | 168537 | 209 | 300 |
| 25 | 7 | 7q36 | 149676368 | 149676891 | ATQL3 | antiquitin-like 3 | 543 | — | — |
| 25 | 7 | 7q36.1 | 149702106 | 149708689 | GIMAP4 | GTPase, IMAP family member 4 | 55303 | 301 | 302 |
| 25 | 7 | 7q36.1 | 149820442 | 149828373 | GIMAP2 | GTPase, IMAP family member 2 | 26157 | 303 | 304 |
| 25 | 7 | 7q36.1 | 149851346 | 149855901 | GIMAP1 | GTPase, IMAP family member 1 | 170575 | 305 | 306 |
| 25 | 7 | 7q36.1 | 149872148 | 149878384 | GIMAP5 | GTPase, IMAP family member 5 | 55340 | 307 | 308 |
| 25 | 7 | 7q36.1 | 149926038 | 149936011 | LR8 | LR8 protein | 28959 | 309 | 310 |
| 25 | 7 | 7q36.1 | 149935502 | 149939856 | HCA112 | hepatocellular carcinoma- | 55365 | 311 | 312 |
| | | | | | | associated antigen 112 | | | |
| 26 | 7 | 7q35-q36 | 150079698 | 150112662 | KCNH2 | potassium voltage-gated | 3757 | 313, 315, 317 | 314, 316, 318 |
| | | | | | | channel, subfamily H (eag- | | | |
| | | | | | | related), member 2 | | | |
| 26 | 7 | 7q36 | 150125796 | 150149324 | NOS3 | nitric oxide synthase 3 | 4846 | 319 | 320 |
| | | | | | | (endothelial cell) | | | |
| 26 | 7 | 7q36 | 150163185 | 150180300 | ABCB8 | ATP-binding cassette, sub-family | 11194 | 321 | 322 |
| | | | | | | B (MDR/TAP), member 8 | | | |
| 26 | 7 | 7q35 | 150183600 | 150187489 | ACCN3 | amiloride-sensitive cation | 9311 | 323, 325, 327 | 324, 326, 328 |
| | | | | | | channel 3 | | | |
| 26 | 7 | 7q36 | 150188547 | 150192644 | CDK5 | cyclin-dependent kinase 5 | 1020 | 329 | 330 |
| 26 | 7 | 7q35-q36 | 150194387 | 150211258 | SLC4A2 | solute carrier family 4, anion | 6522 | 331 | 332 |
| | | | | | | exchanger, member 2 | | | |
| | | | | | | (erythrocyte membrane protein | | | |
| | | | | | | band 3-like 1) | | | |
| 26 | 7 | 7q35 | 150211356 | 150215599 | FASTK | Fas-activated serine/threonine | 10922 | 333, 335 | 334, 336 |
| | | | | | | kinase | | | |
| 26 | 7 | 7q36.1 | 150215821 | 150217736 | C7orf21 | chromosome 7 open reading | 83590 | 337 | 338 |
| | | | | | | frame 21 | | | |
| 26 | 7 | 7q36.1 | 150221474 | 150279171 | CENTG3 | centaurin, gamma 3 | 116988 | 339 | 340 |
| 26 | 7 | 7q36.1 | 150310433 | 150322126 | ASB10 | ankyrin repeat and SOCS box- | 136371 | 341 | 342 |
| | | | | | | containing 10 | | | |
| 26 | 7 | 7q36.1 | 150325611 | 150340230 | LOC346545 | similar to IQ motif containing with | 346545 | 343 | 344 |
| | | | | | | AAA domain | | | |
| 26 | 7 | 7q36 | 150342571 | 150361965 | ABCF2 | ATP-binding cassette, sub-family | 10061 | 345, 347 | 346, 348 |
| | | | | | | F (GCN20), member 2 | | | |
| 26 | 7 | 7q36.1 | 150367223 | 150373553 | CSGlcA-T | chondroitin sulfate | 54480 | 349 | 350 |
| | | | | | | glucuronyltransferase | | | |
| 26 | 7 | 7q35-q36 | 150373707 | 150409988 | SMARCD3 | SWI/SNF related, matrix | 6604 | 351, 353, 355 | 352, 354, 356 |
| | | | | | | associated, actin dependent | | | |
| | | | | | | regulator of chromatin, subfamily | | | |
| | | | | | | d, member 3 | | | |
| 26 | 7 | 7q36 | 150476506 | 150513183 | NYREN18 | NEDD8 ultimate buster-1 | 51667 | 357 | 358 |
| 26 | 7 | 7q36.1 | 150515862 | 150545388 | LOC349136 | hypothetical protein LOC349136 | 349136 | 359 | 360 |
| 27 | 7 | 7q36.3 | 156629186 | 156709609 | DNAJB6 | DnaJ (Hsp40) homolog, | 10049 | 361, 363 | 362, 364 |
| | | | | | | subfamily B, member 6 | | | |
| 27 | 7 | 7q36 | 156831231 | 157879894 | PTPRN2 | protein tyrosine phosphatase, | 5799 | 365, 367, 369 | 366, 368, 370 |
| | | | | | | receptor type, N polypeptide 2 | | | |
| 27 | 7 | 7q36.3 | 157912773 | 157915710 | LOC285872 | THAP5 pseudogene | 285872 | — | — |
| 27 | 7 | 7q36.3 | 157936457 | 157996965 | LUZP5 | leucine zipper protein 5 | 54892 | 371 | 372 |
| 27 | 7 | 7q36.3 | 158023165 | 158121795 | FAM62B | family with sequence similarity 62 | 57488 | 373 | 374 |
| | | | | | | (C2 domain containing) member B | | | |
| 28 | 9 | 9p21.2 | 26831252 | 26882800 | C9orf82 | chromosome 9 open reading | 79886 | 375 | 376 |
| | | | | | | frame 82 | | | |
| 28 | 9 | 9p21 | 26894518 | 26925207 | PLAA | phospholipase A2-activating | 9373 | 377, 379 | 378, 380 |
| | | | | | | protein | | | |
| 28 | 9 | 9p21.2 | 26946410 | 27052802 | IFT74 | intraflagellar transport 74 | 80173 | 381 | 382 |
| | | | | | | homolog (Chlamydomonas) | | | |
| 28 | 9 | 9p21.2 | 26983588 | 26995670 | LRRC19 | leucine rich repeat containing 19 | 64922 | 383 | 384 |
| 28 | 9 | 9p21 | 27099441 | 27220165 | TEK | TEK tyrosine kinase, endothelial | 7010 | 385 | 386 |
| | | | | | | (venous malformations, multiple | | | |
| | | | | | | cutaneous and mucosal) | | | |
| 28 | 9 | 9p21 | 27235682 | 27272791 | C9orf14 | chromosome 9 open reading | 158035 | 387 | 388 |
| | | | | | | frame 14 | | | |
| 28 | 9 | 9p21 | 27274667 | 27287137 | C9orf11 | chromosome 9 open reading | 54586 | 389 | 390 |
| | | | | | | frame 11 | | | |
| 29 | 9 | 9q21.11 | 70103452 | 70196890 | SMC5L1 | SMC5 structural maintenance of | 23137 | 391 | 392 |
| | | | | | | chromosomes 5-like 1 (yeast) | | | |
| 29 | 9 | 9q13 | 70229379 | 70259094 | KLF9 | Kruppel-like factor 9 | 687 | 393 | 394 |
| 29 | 9 | 9q21.11-q21.12 | 70380423 | 70713500 | TRPM3 | transient receptor potential cation | 80036 | 395, 397, 399, 401, 403, 405, | 396, 398, 400, 402, 404, |
| | | | | | | channel, subfamily M, member 3 | | 407, 409, 411 | 406, 408, 410, 412 |
| 30 | 9 | 9q22.31 | 93288338 | 93295429 | C9orf10OS | chromosome 9 open reading | 158293 | 413 | 414 |
| | | | | | | frame 10 opposite strand | | | |
| 30 | 9 | 9q22.31 | 93293728 | 93407949 | C9orf10 | chromosome 9 open reading | 23196 | 415 | 416 |
| | | | | | | frame 10 | | | |
| 30 | 9 | 9q22.31 | 93418585 | 93521421 | PHF2 | PHD finger protein 2 | 5253 | 417, 419 | 418, 420 |
| 31 | 9 | 9q31.3-q32 | 110510694 | 110642833 | MUSK | muscle, skeletal, receptor | 4593 | 421 | 422 |
| | | | | | | tyrosine kinase | | | |
| 31 | 9 | 9q31.3 | 110715611 | 110879920 | EDG2 | endothelial differentiation, | 1902 | 423, 425 | 424, 426 |
| | | | | | | lysophosphatidic acid G-protein- | | | |
| | | | | | | coupled receptor, 2 | | | |
| 32 | 10 | 10p12.33-p12.32 | 19432550 | 20145269 | C10orf112 | chromosome 10 open reading | 340895 | 427 | 428 |
| | | | | | | frame 112 | | | |
| 32 | 10 | 10p12.32-p12.31 | 20145378 | 20609121 | PLXDC2 | plexin domain containing 2 | 84898 | 429 | 430 |
| 33 | 10 | 10q24.2 | 99905840 | 99994644 | C10orf28 | chromosome 10 open reading | 27291 | 431 | 432 |
| | | | | | | frame 28 | | | |
| 33 | 10 | 10q24 | 99997440 | 100017997 | LOXL4 | lysyl oxidase-like 4 | 84171 | 433 | 434 |
| 33 | 10 | 10q24.2 | 100133314 | 100164931 | C10orf33 | chromosome 10 open reading | 84795 | 435 | 436 |
| | | | | | | frame 33 | | | |
| 33 | 10 | 10q23.1-q23.3 | 100165646 | 100196694 | HPS1 | Hermansky-Pudlak syndrome 1 | 3257 | 437, 439, 441, 443 | 438, 440, 442, 444 |
| 33 | 10 | 10q23-q24 | 100208865 | 100985609 | HPSE2 | heparanase-2 | 60495 | 445 | 446 |
| 33 | 10 | 10q24.2 | 101080023 | 101144077 | CNNM1 | cyclin M1 | 26507 | 447 | 448 |
| 33 | 10 | 10q24.1-q25.1 | 101146618 | 101180336 | GOT1 | glutamic-oxaloacetic | 2805 | 449 | 450 |
| | | | | | | transaminase 1, soluble | | | |
| | | | | | | (aspartate aminotransferase 1) | | | |
| 34 | 11 | 11p15.3 | 11820141 | 11934691 | USP47 | ubiquitin specific peptidase 47 | 55031 | 451 | 452 |
| 34 | 11 | 11p15.2 | 11941229 | 11987493 | DKK3 | dickkopf homolog 3 (Xenopus | 27122 | 453, 455, 457 | 454, 456, 458 |
| | | | | | | laevis) | | | |
| 34 | 11 | 11p15.3 | 12088714 | 12241908 | MICAL2 | microtubule associated | 9645 | 459 | 460 |
| | | | | | | monoxygenase, calponin and | | | |
| | | | | | | LIM domain containing 2 | | | |
| 35 | 11 | 11p15.1 | 19691488 | 20009720 | NAV2 | neuron navigator 2 | 89797 | 461, 463 | 462, 464 |
| 35 | 11 | 11p15.1 | 20134336 | 20138446 | DBX1 | developing brain homeobox 1 | 120237 | 465 | 466 |
| 36 | 11 | 11q21 | 95141766 | 95162429 | FAM76B | family with sequence similarity | 143684 | 467 | 468 |
| | | | | | | 76, member B | | | |
| 36 | 11 | 11q21 | 95163290 | 95204359 | CEP57 | centrosomal protein 57 kDa | 9702 | 469 | 470 |
| 36 | 11 | 11q22 | 95205694 | 95296920 | MTMR2 | myotubularin related protein 2 | 8898 | 471, 473, 475 | 472, 474, 476 |
| 36 | 11 | 11q21 | 95351088 | 95715992 | MAML2 | mastermind-like 2 (Drosophila) | 84441 | 477 | 478 |
| 36 | 11 | 11q21 | 95725590 | 95762709 | CCDC82 | hypothetical protein FLJ23518 | 79780 | 479 | 480 |
| 36 | 11 | 11q21 | 95763462 | 95766375 | JRKL | jerky homolog-like (mouse) | 8690 | 481 | 482 |
| 37 | 12 | 12q13.1 | 56114810 | 56130876 | INHBC | inhibin, beta C | 3626 | 483 | 484 |
| 37 | 12 | 12q13.3 | 56135363 | 56138058 | INHBE | inhibin, beta E | 83729 | 485 | 486 |
| 37 | 12 | 12q13.2-q13.3 | 56140201 | 56152312 | GLI1 | glioma-associated oncogene | 2735 | 487 | 488 |
| | | | | | | homolog 1 (zinc finger protein) | | | |
| 37 | 12 | 12q14 | 56152314 | 56168864 | ARHGAP9 | Rho GTPase activating protein 9 | 64333 | 489 | 490 |
| 37 | 12 | 12q13.2 | 56168118 | 56196700 | MARS | methionine-tRNA synthetase | 4141 | 491 | 492 |
| 37 | 12 | 12q13.1-q13.2 | 56196643 | 56200567 | DDIT3 | DNA-damage-inducible transcript 3 | 1649 | 493 | 464 |
| 37 | 12 | 12q13.1-q13.2 | 56202926 | 56210198 | MBD6 | methyl-CpG binding domain | 114785 | 495 | 496 |
| | | | | | | protein 6 | | | |
| 37 | 12 | 12q13.2-q13.3 | 56210361 | 56227245 | DCTN2 | dynactin 2 (p50) | 10540 | 497 | 498 |
| 37 | 12 | 12q13.13 | 56230114 | 56264821 | KIF5A | kinesin family member 5A | 3798 | 499 | 500 |
| 37 | 12 | 12q13.3 | 56271324 | 56283298 | PIP5K2C | phosphatidylinositol-4-phosphate | 79837 | 501 | 502 |
| | | | | | | 5-kinase, type II, gamma | | | |
| 37 | 12 | 12q13.3 | 56284871 | 56289850 | DTX3 | deltex 3 homolog (Drosophila) | 196403 | 503 | 504 |
| 37 | 12 | 12q13.3 | 56291485 | 56297293 | GEFT | RAC/CDC42 exchange factor | 115557 | 505, 507 | 506, 508 |
| 37 | 12 | 12q13 | 56301135 | 56306201 | SLC26A10 | solute carrier family 26, member | 65012 | 509, 511 | 510, 512 |
| | | | | | | 10 | | | |
| 37 | 12 | 12q13.3 | 56305945 | 56313251 | B4GALNT1 | beta-1,4-N-acetyl-galactosaminyl | 2583 | 513 | 514 |
| | | | | | | transferase 1 | | | |
| 37 | 12 | 12q14.1 | 56312918 | 56355300 | LOC441641 | similar to ribosomal protein L13A | 441641 | — | — |
| 37 | 12 | 12q13 | 56374187 | 56401606 | OS9 | amplified in osteosarcoma | 10956 | 515, 517, 519, 521 | 516, 518, 520, 522 |
| 37 | 12 | 12q14.1 | 56405261 | 56422207 | CENTG1 | centaurin, gamma 1 | 116986 | 523 | 524 |
| 37 | 12 | 12q13.3 | 56425051 | 56428293 | TSPAN31 | tetraspanin 31 | 6302 | 525 | 526 |
| 37 | 12 | 12q14 | 56428270 | 56432431 | CDK4 | cyclin-dependent kinase 4 | 1019 | 527 | 528 |
| 37 | 12 | 12q14.1 | 56435167 | 56439466 | MARCH9 | membrane-associated ring finger | 92979 | 529 | 530 |
| | | | | | | (C3HC4) 9 | | | |
| 37 | 12 | 12q13.1-q13.3 | 56442386 | 56447151 | CYP27B1 | cytochrome P450, family 27, | 1594 | 531 | 532 |
| | | | | | | subfamily B, polypeptide 1 | | | |
| 37 | 12 | 12q13 | 56448523 | 56452522 | METTL1 | methyltransferase like 1 | 4234 | 533, 535, 537 | 534, 536, 538 |
| 37 | 12 | 12q14.1 | 56452650 | 56462591 | DKFZP586D0919 | hepatocellularcarcinoma- | 25895 | 539, 541 | 540, 542 |
| | | | | | | associated antigen HCA557a | | | |
| 37 | 12 | 12q13-q14 | 56462826 | 56476784 | TSFM | Ts translation elongation factor, | 10102 | 543 | 544 |
| | | | | | | mitochondrial | | | |
| 37 | 12 | 12q14.1 | 56477710 | 56496119 | AVIL | advillin | 10677 | 545 | 546 |
| 37 | 12 | 12q13-q15 | 56499977 | 56526789 | CTDSP2 | CTD (carboxy-terminal domain, | 10106 | 547 | 548 |
| | | | | | | RNA polymerase II, polypeptide | | | |
| | | | | | | A) small phosphatase 2 | | | |
| 37 | 12 | 12q14.1 | 56621711 | 56637319 | XRCC6BP1 | XRCC6 binding protein 1 | 91419 | 549 | 550 |
| 38 | 12 | 12q21.31 | 81254792 | 81375413 | C12orf26 | chromosome 12 open reading | 84190 | 551 | 552 |
| | | | | | | frame 26 | | | |
| 38 | 12 | 12q21.31 | 81583402 | 82030533 | TMTC2 | transmembrane and | 160335 | 553 | 554 |
| | | | | | | tetratricopeptide repeat | | | |
| | | | | | | containing 2 | | | |
| 38 | 12 | 12q21.31 | 82047578 | 82048437 | LOC401725 | similar to 60S ribosomal protein | 401725 | — | — |
| | | | | | | L6 (TAX-responsive enhancer | | | |
| | | | | | | element binding protein 107) | | | |
| | | | | | | (TAXREB107) (Neoplasm-related | | | |
| | | | | | | protein C140) | | | |
| 39 | 14 | 14q22.1 | 49620119 | 49629111 | LOC196913 | hypothetical protein LOC196913 | 196913 | 555 | 556 |
| 39 | 14 | 14q22.1 | 49645106 | 49653047 | C14orf138 | chromosome 14 open reading | 79609 | 557 | 558 |
| | | | | | | frame 138 | | | |
| 39 | 14 | 14q21 | 49654812 | 49767751 | SOS2 | son of sevenless homolog 2 | 6655 | 559 | 560 |
| | | | | | | (Drosophila) | | | |
| 39 | 14 | 14q22.1 | 49782933 | 49848697 | L2HGDH | L-2-hydroxyglutarate | 79944 | 561 | 562 |
| | | | | | | dehydrogenase | | | |
| 39 | 14 | 14q22.1 | 49848852 | 49859363 | ATP5S | ATP synthase, H+ transporting, | 27109 | 563, 565, 567 | 564, 566, 568 |
| | | | | | | mitochondrial F0 complex, | | | |
| | | | | | | subunit s (factor B) | | | |
| 39 | 14 | 14q22.1 | 49866470 | 49932367 | CDKL1 | cyclin-dependent kinase-like 1 | 8814 | 569 | 570 |
| | | | | | | (CDC2-related kinase) | | | |
| 39 | 14 | 14q11.2-q21 | 49954993 | 50069126 | MAP4K5 | mitogen-activated protein kinase | 11183 | 571, 573 | 572, 574 |
| | | | | | | kinase kinase kinane 5 | | | |
| 39 | 14 | 14q22.1 | 50096514 | 50169536 | SPG3A | spastic paraplegia 3A (autosomal | 51062 | 575, 577 | 576, 578 |
| | | | | | | dominant) | | | |
| 39 | 14 | 14q22.1 | 50108247 | 50108634 | SNRPGP | small nuclear ribonucleoprotein | 326272 | — | — |
| | | | | | | polypeptide G pseudogene | | | |
| 39 | 14 | 14q13-q23 | 50170110 | 50204773 | SAV1 | salvador homolog 1 (Drosophila) | 60485 | 579 | 580 |
| 40 | 14 | 14q31.1 | 80358603 | 80359699 | HMGN2P2 | high-mobility group nucleosomal | 317726 | — | — |
| | | | | | | binding domain 2 pseudogene 2 | | | |
| 40 | 14 | 14q31 | 80491679 | 80680525 | TSHR | thyroid stimulating hormone | 7253 | 581, 583 | 582, 584 |
| | | | | | | receptor | | | |
| 40 | 14 | 14q31.1 | 80537231 | 80537914 | RPL17P3 | ribosomal protein L17 | 326286 | — | — |
| | | | | | | pseudogene 3 | | | |
| 40 | 14 | 14q31.1 | 80568571 | 80569637 | NMNATP | nicotinamide nucleotide | 326607 | — | — |
| | | | | | | adenylyltransferase pseudogene | | | |
| 40 | 14 | 14q31.1 | 80716147 | 80757328 | GTF2A1 | general transcription factor IIA, 1, | 2957 | 585, 587 | 586, 588 |
| | | | | | | 19/37 kDa | | | |
| 40 | 14 | 14q31.1 | 80782113 | 80782751 | LOC246720 | dynein, cytoplasmic, light | 246720 | — | — |
| | | | | | | polypeptide pseudogene | | | |
| 40 | 14 | 14q31.1 | 80794017 | 80796268 | UNGP3 | uracil-DNA glycosylase | 319122 | — | — |
| | | | | | | pseudogene 3 | | | |
| 40 | 14 | 14q31.1 | 80806662 | 80934680 | STON2 | stonin 2 | 85439 | 589 | 590 |
| 40 | 14 | 14q31.1 | 80868934 | 80869490 | RPS24P3 | ribosomal protein S24 | 326322 | — | — |
| | | | | | | pseudogene 3 | | | |
| 41 | 16 | 16q12.1 | 46674385 | 46738182 | ABCC12 | ATP-binding cassette, sub-family | 94160 | 591 | 592 |
| | | | | | | C (CFTR/MRP), member 12 | | | |
| 41 | 16 | 16q12.1 | 46758323 | 46826589 | ABCC11 | ATP-binding cassette, sub-family | 85320 | 593, 595, 597 | 594, 596, 598 |
| | | | | | | C (CFTR/MRP), member 11 | | | |
| 41 | 16 | 16q12.1 | 46835712 | 46944908 | LONPL | peroxisomal LON protease like | 83752 | 599 | 600 |
| 41 | 16 | 16q12 | 46951950 | 46954901 | SIAH1 | seven in absentia homolog 1 | 6477 | 601, 603 | 602, 604 |
| | | | | | | (Drosophila) | | | |
| 42 | 16 | 16q23.1 | 73590416 | 73702393 | ZNRF1 | zinc and ring finger 1 | 84937 | 605 | 606 |
| 42 | 16 | 16q23.1 | 73703260 | 73708166 | LDHD | lactate dehydrogenase D | 197257 | 607, 609 | 608, 610 |
| 42 | 16 | 16q23.1 | 73739926 | 73763486 | ZFP1 | zinc finger protein 1 homolog | 162239 | 611 | 612 |
| | | | | | | (mouse) | | | |
| 42 | 16 | 16q23.1 | 73783375 | 73784646 | LOC441774 | similar to 40S ribosomal protein | 441774 | — | — |
| | | | | | | S4, Y isoform 1 | | | |
| 42 | 16 | 16q23.1 | 73795499 | 73798582 | CTRB2 | chymotrypsinogen B2 | 440387 | 613 | 614 |
| 42 | 16 | 16q23-q24.1 | 73810399 | 73816322 | CTRB1 | chymotrypsinogen B1 | 1504 | 615 | 616 |
| 42 | 16 | 16q22-q23 | 73820430 | 73843004 | BCAR1 | breast cancer anti-estrogen | 9564 | 617 | 618 |
| | | | | | | resistance 1 | | | |
| 42 | 16 | 16q22.2-q22.3 | 73885116 | 74024860 | CFDP1 | craniofacial development protein 1 | 10428 | 619 | 620 |
| 42 | 16 | 16q23.1 | 74038426 | 74056085 | LOC124491 | LOC124491 | 124491 | 621 | 622 |
| 42 | 16 | 16q22 | 74064526 | 74086428 | CHST6 | carbohydrate (N- | 4166 | 623 | 624 |
| | | | | | | acetylglucosamine 6-O) | | | |
| | | | | | | sulfotransferase 6 | | | |
| 43 | 16 | 16q23.1 | 76379984 | 76571505 | KIAA1576 | KIAA1576 protein | 57687 | 625 | 626 |
| 43 | 16 | 16q23 | 76613946 | 76622239 | CLEC3A | C-type lectin domain family 3, | 10143 | 627 | 628 |
| | | | | | | member A | | | |
| 43 | 16 | 16q23.1 | 76639896 | 76649857 | LOC342419 | similar to Keratin, type II | 342419 | — | — |
| | | | | | | cytoskeletal 8 (Cytokeratin-8) | | | |
| | | | | | | (CK-8) (Keraton-8) (K8) | | | |
| | | | | | | (Cytokeratin endo A) | | | |
| 43 | 16 | 16q23.3-q24.1 | 76691052 | 77804065 | WWOX | WW domain containing | 51741 | 629, 631, 633 | 630, 632, 634 |
| | | | | | | oxidoreductase | | | |
| 44 | 18 | 18q11.2 | 22665430 | 22672683 | LOC440489 | ubiquitin fusion degradation 1- | 440489 | — | — |
| | | | | | | like pseudogene | | | |
| 44 | 18 | 18q11.2-q12.1 | 22686008 | 22699699 | AQP4 | aquaporin 4 | 361 | 635, 637 | 636, 638 |
| 44 | 18 | 18q11.2 | 22699270 | 22769908 | C18orf16 | chromosome 18 open reading | 147429 | 639 | 640 |
| | | | | | | frame 16 | | | |
| 44 | 18 | 18q11.2 | 22749595 | 23019177 | CHST9 | carbohydrate (N- | 83539 | 641 | 642 |
| | | | | | | acetylgalactosamine 4-0) | | | |
| | | | | | | sulfotransferase 9 | | | |
| 44 | 18 | 18q11.2-q12.1 | 23170341 | 23429126 | FLJ45994 | hypothetical gene supported by | 400645 | 643 | 644 |
| | | | | | | AK127888 | | | |
| 45 | 18 | 18q22-q23 | 61568468 | 61699155 | CDH7 | cadherin 7, type 2 | 1005 | 645, 647 | 646, 648 |
| 46 | 22 | 22pter-q11.2\|22q11.1 | 16449479 | 16486089 | ATP6V1E1 | ATPase, H+ transporting, | 529 | 649, 651, 653 | 650, 652, 654 |
| | | | | | | lysosomal 31 kDa, V1 subunit E1 | | | |
| 46 | 22 | 22q11 | 16496039 | 16586545 | BCL2L13 | BCL2-like 13 (apoptosis | 23786 | 655 | 656 |
| | | | | | | facilitator) | | | |
| 46 | 22 | 22q11.1 | 16591460 | 16631812 | BID | BH3 interacting domain death | 637 | 657, 659, 661 | 658, 660, 662 |
| | | | | | | agonist | | | |
| 46 | 22 | 22q11.21 | 16697271 | 16764155 | MICAL3 | microtubule associated | 57553 | 663, 665, 667, 669, 671, 673 | 664, 666, 668, 670, 672, |
| | | | | | | monoxygenase, calponin and | | | 674 |
| | | | | | | LIM domain containing 3 | | | |
| 46 | 22 | 22q11.21 | 16935243 | 16948790 | PEX26 | peroxisome biogenesis factor 26 | 55670 | 675 | 676 |
| 46 | 22 | 22q11.1 | 16968113 | 16989052 | TUBA8 | tubulin, alpha 8 | 51807 | 677 | 678 |
| 47 | X | Xq13.3 | 80175345 | 80183261 | NSBP1 | nucleosomal binding protein 1 | 79366 | 679 | 680 |
| 47 | X | Xq13.3 | 80263767 | 80360191 | SH3BGRL | SH3 domain binding glutamic | 6451 | 681 | 682 |
| | | | | | | acid-rich protein like | | | |

TABLE 5
List of additional longevity candidate genes from the genome wide and fine mapping association analyses
derived from B36. In order to identify genes not placed in the regions from Table 1 according to Build 35,
the region coordinates were converted to Build 36 using the UCSC (University of California Santa Cruz) online
program LiftOver. Only new genes that were mapped to this version of the genome asembly are included in this table.
The first column corresponds to the region identifier provided in Table 1. The second and third columns correspond
to the chromosome and cytogenetic band, respectively. The fourth and fifth columns correspond to the chromosomal
start and end coordinates of the NCBI genome assembly derived from build 36 (the start and end position relate
to the + orientation of the NCBI assembly and do not necessarily correspond to the orientation of the gene).
The sixth and seventh columns correspond to the official gene symbol and gene name, respectively, and were obtained
from the NCBI Entrez Gene database. The eighth colums corresponds to the NCBI Entrez Gene Identifier (Gene ID).
The ninth and tenth columns correspond to the Sequence IDs from nucleotide (cDNA) and protein entries in the
Sequence Listing.

| | | | Start | End | | | | | |
|---|---|---|---|---|---|---|---|---|---|
| | | Cytogenetic | Position | position | Gene | | Entrez | | |
| Region ID | Chromosome | Band | B36 | B36 | Symbol | Gene Name | Gene ID | Nucleotide Seq ID | Protein Seq ID |
| 2 | 1 | 1q42 | 227473294 | 227473969 | HRES1 | HTLV-1 related endogenous sequence | 3272 | 683 | 684 |
| 3 | 1 | 1q42.2 | 229223834 | 229226580 | LOC644006 | similar to RING finger protein 4 | 644006 | 685 | 686 |
| 4 | 1 | 1q43 | 238218704 | 238243181 | LOC645884 | similar to 40S ribosomal protein S7 (S8) | 645884 | — | — |
| 8 | 2 | 2q14.3 | 122156781 | 122160491 | LOC646151 | hypothetical protein LOC646151 | 646151 | — | — |
| 8 | 2 | 2q14.3 | 122264517 | 122270396 | LOC646179 | hypothetical protein LOC646179 | 646179 | 687, 689 | 688, 690 |
| 9 | 2 | 2q21.1 | 128033768 | 128111767 | MYO7B | myosin VIIB | 4648 | 691 | 692 |
| 12 | 3 | 3p26.1 | 7969498 | 8032988 | LOC643291 | hypothetical protein LOC643291 | 643291 | 693, 695 | 694, 696 |
| 13 | 3 | 3p22.3 | 33106918 | 33113296 | FLJ45032 | similar to F40B5.2b | 643853 | 697 | 698 |
| 14 | 3 | 3p22.1 | 40603030 | 40605031 | LOC645807 | similar to ribosomal protein L5 | 645807 | — | — |
| 15 | 4 | 4q21.1 | 77176927 | 77177241 | LOC643205 | similar to ribosomal protein L36 | 643205 | 699 | 700 |
| 15 | 4 | 4q21.1 | 77189641 | 77210116 | LOC643214 | hypothetical protein LOC643214 | 643214 | — | — |
| 16 | 4 | 4q23 | 101130917 | 101131660 | LOC644721 | similar to ribosomal protein L7-like 1 | 644721 | — | — |
| 17 | 4 | 4q28.1 | 126863872 | 126869392 | LOC645841 | hypothetical protein LOC645841 | 645841 | — | — |
| 19 | 6 | 6p22.1 | 27121856 | 27122077 | LOC645845 | hypothetical protein LOC645845 | 645845 | 701 | 702 |
| 19 | 6 | 6p22.1 | 27358487 | 27374104 | LOC645898 | similar to SET domain and mariner | 645898 | — | — |
| | | | | | | transposase fusion gene | | | |
| 22 | 7 | 7p14.3 | 33732124 | 33734587 | FLJ20712 | hypothetical protein FLJ20712 | 55025 | 703 | 704 |
| 23 | 7 | 7q32.1 | 127592525 | 127595886 | LOC646893 | hypothetical protein LOC646893 | 646893 | 705, 707 | 706, 708 |
| 23 | 7 | 7q32.1 | 127731406 | 127735094 | MGC27345 | hypothetical protein MGC27345 | 157247 | 709 | — |
| 24 | 7 | 7q36.1 | 147908426 | 147908824 | LOC402716 | similar to 60S ribosomal protein L32 | 402716 | 710 | 711 |
| 24 | 7 | 7q36.1 | 147965225 | 147965617 | tcag7.1239 | Rho guanine nucleotide exchange factor | 643438 | 712, 714 | 713, 715 |
| | | | | | | (GEF) 5 | | | |
| 25 | 7 | 7q36.1 | 149575240 | 149651741 | LOC653722 | similar to actin-related protein 3-beta | 653722 | 716, 718 | 717, 719 |
| 25 | 7 | 7q36.1 | 149941107 | 149942798 | LOC643852 | similar to transient receptor protein 6 | 643852 | — | — |
| 25 | 7 | 7q36.1 | 149953396 | 149960374 | GIMAP6 | GTPase, IMAP family member 6 | 474344 | 720, 722 | 721, 723 |
| 25 | 7 | 7q36.1 | 150074898 | 150075786 | GIMAP3P | GTPase, IMAP family member 3 | 474345 | — | — |
| | | | | | | pseudogene | | | |
| 26 | 7 | 7q36.1 | 150340233 | 150352519 | ATG9B | ATG9 autophagy related 9 homolog B | 285973 | 724 | 725 |
| | | | | | | (S. cerevisiae) | | | |
| 26 | 7 | 7q36.1 | 150476609 | 150495800 | LOC442747 | hypothetical LOC442747 | 442747 | — | — |
| 26 | 7 | 7q36.1 | 150518899 | 150523589 | LOC392843 | similar to IQ motif containing with AAA | 392843 | 726, 728 | 727, 729 |
| | | | | | | domain | | | |
| 27 | 7 | 7q36.3 | 156926773 | 156956238 | LOC393078 | similar to polycystic kidney disease 1 like 3 | 393078 | 730 | 731 |
| 27 | 7 | 7q36.3 | 157346084 | 157347702 | LOC642620 | hypothetical protein LOC642620 | 642620 | 732, 734 | 733, 735 |
| 27 | 7 | 7q36.3 | 158205200 | 158205622 | LOC645373 | similar 60S ribosomal protein L21 | 645373 | 736, 738 | 737, 739 |
| 31 | 9 | 9q32 | 112168283 | 112381975 | SVEP1 | sushi, von Willebrand factor type A, EGF | 79987 | 740, 742, 744, 746, 748, 750 | 741, 743, 745, 747, 749, 751 |
| | | | | | | and pentraxin domain containing 1 | | | |
| 31 | 9 | 9q31.3 | 112774287 | 112840804 | LOC644923 | hypothetical protein LOC644923 | 644923 | 752, 754 | 753, 755 |
| 34 | 11 | 11p15.1 | 11938953 | 11939310 | RIG | regulated in glioma | 10530 | 756 | 757 |
| 34 | 11 | 11p15.3 | 11939324 | 11939651 | LOC644887 | hypothetical protein LOC644887 | 644887 | 758 | 759 |
| 36 | 11 | 11q21 | 95311939 | 95336379 | LOC643259 | similar to Periphilin 1 (Gastric cancer | 643259 | — | — |
| | | | | | | antigen Ga50) | | | |
| 36 | 11 | 11q21 | 95627227 | 95630536 | LOC645238 | hypothetical protein LOC645238 | 645238 | 760, 762 | 761, 763 |
| 40 | 14 | 14q31.1 | 80032577 | 80475637 | C14orf145 | chromosome 14 open reading frame 145 | 145508 | 764 | 765 |
| 42 | 16 | 16q23.1 | 74086403 | 74105239 | LOC645799 | similar to carbohydrate (N- | 645799 | 766, 768 | 767, 769 |
| | | | | | | acetylglucosamine 6-O) sulfotransferase 5 | | | |
| 43 | 16 | 16q23.1 | 76946927 | 76947232 | LOC645947 | similar to LSM3 homolog, U6 small | 645947 | 770 | 771 |
| | | | | | | nuclear RNA associated | | | |
| 43 | 16 | 16q23.1 | 77416205 | 77417404 | LOC645957 | similar to 40S ribosomal protein S3 | 645957 | — | — |
| 45 | 18 | 18q22.1 | 61490076 | 61491640 | LOC643448 | hypothetical protein LOC643448 | 643448 | 772, 774 | 773, 775 |
| 46 | 22 | 22q11.21 | 16697107 | 16864261 | LOC642566 | similar to Protein MICAL-3 | 642566 | — | — |

TABLE 6
List of candidate genes based on EST clustering from the regions identified from the various Fine Mapping
analyses. The first column corresponds to the region identifier provided in Table 1. The second
column corresponds to the chromosome number. The third and fourth columns correspond to the chromosomal
start and end coordinates of the NCBI genome assemblies derived from build 35 (B35). The fifth
column corresponds to the ECGene Identifier, corresponding to the ECGene track of UCSC. These
ECGene entries were determined by their overlap with the regions from Table 1, based on the start
and end coordinates of both Region and ECGene identifiers. The sixth and seventh columns correspond
to the Sequence IDs from nucleotide and protein entries in the Sequence Listing.

| Region ID | Chromosome | Start Position Build35 | End Position Build35 | Name | Nucleotide Seq ID | Protein Seq ID |
|---|---|---|---|---|---|---|
| 1 | 1 | 172908440 | 172930123 | H1C20934.1 | 776 | 777 |
| 1 | 1 | 173163963 | 173256767 | H1C20945.1 | 778 | 779 |
| 1 | 1 | 173163963 | 173391998 | H1C20945.2 | 780 | 781 |
| 1 | 1 | 173163963 | 173543625 | H1C20945.3 | 782 | 783 |
| 1 | 1 | 173254289 | 173258062 | H1C20945.4 | 784 | 785 |
| 1 | 1 | 173544405 | 173546396 | H1C21012.1 | 786 | 787 |
| 1 | 1 | 173558095 | 173865681 | H1C21015.1 | 788 | 789 |
| 1 | 1 | 173561725 | 173882650 | H1C21015.2 | 790 | 791 |
| 1 | 1 | 173561856 | 173865681 | H1C21015.3 | 792 | 793 |
| 1 | 1 | 173561856 | 173882650 | H1C21015.4 | 794 | 795 |
| 1 | 1 | 173569636 | 173865681 | H1C21015.5 | 796 | 797 |
| 1 | 1 | 173676847 | 173733572 | H1C21015.6 | 798 | 799 |
| 2 | 1 | 225465254 | 225480756 | H1C26521.1 | 800 | 801 |
| 2 | 1 | 225535152 | 225562963 | H1C26524.1 | 802 | 803 |
| 2 | 1 | 225544148 | 225686077 | H1C26525.1 | 804 | 805 |
| 2 | 1 | 225665942 | 225670044 | H1C26533.1 | 806 | 807 |
| 2 | 1 | 225711293 | 225713506 | H1C26538.1 | 808 | 809 |
| 2 | 1 | 225713543 | 225746488 | H1C26539.1 | 810 | 811 |
| 2 | 1 | 225713543 | 225746889 | H1C26539.2 | 812 | 813 |
| 2 | 1 | 225713543 | 225747252 | H1C26539.3 | 814 | 815 |
| 2 | 1 | 225713543 | 225747986 | H1C26539.4 | 816 | 817 |
| 2 | 1 | 225713556 | 225731364 | H1C26539.5 | 818 | 819 |
| 2 | 1 | 225713613 | 225746889 | H1C26539.6 | 820 | 821 |
| 2 | 1 | 225713613 | 225747986 | H1C26539.7 | 822 | 823 |
| 2 | 1 | 225713791 | 225746488 | H1C26539.8 | 824 | 825 |
| 2 | 1 | 225714835 | 225716387 | H1C26540.1 | 826 | 827 |
| 2 | 1 | 225746082 | 225766672 | H1C26553.1 | 828 | 829 |
| 2 | 1 | 225747015 | 225747983 | H1C26554.1 | 830 | 831 |
| 2 | 1 | 225761884 | 225765554 | H1C26553.2 | 832 | 833 |
| 2 | 1 | 225763487 | 225765652 | H1C26557.1 | 834 | 835 |
| 2 | 1 | 225763487 | 225783115 | H1C26558.1 | 836 | 837 |
| 2 | 1 | 225763487 | 225785035 | H1C26558.2 | 838 | 839 |
| 2 | 1 | 225766021 | 225785776 | H1C26558.3 | 840 | 841 |
| 2 | 1 | 225766949 | 225785776 | H1C26558.4 | 842 | 843 |
| 2 | 1 | 225873726 | 225874322 | H1C26573.1 | 844 | 845 |
| 2 | 1 | 225873726 | 225874662 | H1C26574.1 | 846 | 847 |
| 2 | 1 | 225873726 | 225875353 | H1C26574.2 | 848 | 849 |
| 2 | 1 | 225873726 | 225875353 | H1C26574.3 | 850 | 851 |
| 2 | 1 | 225873726 | 225875364 | H1C26574.4 | 852 | 853 |
| 2 | 1 | 225873726 | 225876576 | H1C26574.5 | 854 | 855 |
| 2 | 1 | 225873726 | 225876576 | H1C26574.6 | 856 | 857 |
| 2 | 1 | 225873726 | 225876580 | H1C26574.7 | 858 | 859 |
| 2 | 1 | 225874360 | 225874800 | H1C26574.8 | 860 | 861 |
| 2 | 1 | 225874473 | 225874813 | H1C26574.9 | 862 | 863 |
| 2 | 1 | 225874473 | 225875364 | H1C26574.10 | 864 | 865 |
| 2 | 1 | 225874825 | 225876579 | H1C26574.11 | 866 | 867 |
| 2 | 1 | 225882231 | 225883497 | H1C26578.1 | 868 | 869 |
| 2 | 1 | 225882231 | 225893233 | H1C26579.1 | 870 | 871 |
| 2 | 1 | 225882231 | 225903212 | H1C26579.2 | 872 | 873 |
| 2 | 1 | 225882231 | 225950843 | H1C26579.3 | 874 | 875 |
| 2 | 1 | 225882231 | 225950843 | H1C26579.4 | 876 | 877 |
| 2 | 1 | 225883770 | 225893233 | H1C26579.5 | 878 | 879 |
| 2 | 1 | 225883770 | 225903212 | H1C26579.6 | 880 | 881 |
| 2 | 1 | 225883770 | 225950843 | H1C26579.7 | 882 | 883 |
| 2 | 1 | 225884183 | 225893233 | H1C26579.8 | 884 | 885 |
| 2 | 1 | 225884183 | 225903212 | H1C26579.9 | 886 | 887 |
| 2 | 1 | 225884222 | 225893233 | H1C26579.10 | 888 | 889 |
| 2 | 1 | 225884222 | 225903212 | H1C26579.11 | 890 | 891 |
| 2 | 1 | 225884222 | 225950843 | H1C26579.12 | 892 | 893 |
| 2 | 1 | 225891416 | 225893552 | H1C26579.13 | 894 | 895 |
| 2 | 1 | 225941635 | 225943988 | H1C26579.14 | 896 | 897 |
| 2 | 1 | 225950873 | 225956752 | H1C26595.1 | 898 | 899 |
| 2 | 1 | 225950965 | 225956746 | H1C26595.2 | 900 | 901 |
| 2 | 1 | 225958993 | 225961481 | H1C26596.1 | 902 | 903 |
| 2 | 1 | 225958993 | 226001177 | H1C26596.2 | 904 | 905 |
| 2 | 1 | 225959064 | 225961481 | H1C26596.3 | 906 | 907 |
| 2 | 1 | 225959064 | 226001177 | H1C26596.4 | 908 | 909 |
| 2 | 1 | 225959325 | 226001172 | H1C26596.5 | 910 | 911 |
| 2 | 1 | 225974765 | 225976334 | H1C26607.1 | 912 | — |
| 3 | 1 | 227084961 | 227103354 | H1C26754.1 | 913 | 914 |
| 3 | 1 | 227084961 | 227125890 | H1C26754.2 | 915 | 916 |
| 3 | 1 | 227084961 | 227136466 | H1C26754.3 | 917 | 918 |
| 3 | 1 | 227130468 | 227132744 | H1C26754.4 | 919 | 920 |
| 3 | 1 | 227132458 | 227134196 | H1C26754.5 | 921 | 922 |
| 3 | 1 | 227144623 | 227156778 | H1C26767.1 | 923 | 924 |
| 3 | 1 | 227145007 | 227153336 | H1C26768.1 | 925 | 926 |
| 3 | 1 | 227145008 | 227156778 | H1C26767.2 | 927 | 928 |
| 3 | 1 | 227145210 | 227145757 | H1C26768.2 | 929 | 930 |
| 3 | 1 | 227145210 | 227153336 | H1C26768.3 | 931 | 932 |
| 3 | 1 | 227145210 | 227156778 | H1C26767.3 | 933 | 934 |
| 3 | 1 | 227189864 | 227244253 | H1C26778.1 | 935 | 936 |
| 3 | 1 | 227189874 | 227244590 | H1C26778.2 | 937 | 938 |
| 3 | 1 | 227189912 | 227244253 | H1C26778.3 | 939 | 940 |
| 3 | 1 | 227218611 | 227237974 | H1C26767.4 | 941 | 942 |
| 3 | 1 | 227266124 | 227267188 | H1C26784.1 | 943 | 944 |
| 3 | 1 | 227279599 | 227280863 | H1C26787.1 | 945 | 946 |
| 3 | 1 | 227279599 | 227311207 | H1C26789.1 | 947 | 948 |
| 3 | 1 | 227279599 | 227312066 | H1C26789.2 | 949 | 950 |
| 3 | 1 | 227310663 | 227312051 | H1C26789.3 | 951 | 952 |
| 3 | 1 | 227311632 | 227317273 | H1C26794.1 | 953 | 954 |
| 3 | 1 | 227317326 | 227321496 | H1C26796.1 | 955 | 956 |
| 3 | 1 | 227348723 | 227421319 | H1C26803.1 | 957 | 958 |
| 3 | 1 | 227348723 | 227421341 | H1C26803.2 | 959 | 960 |
| 3 | 1 | 227350117 | 227351274 | H1C26804.1 | 961 | 962 |
| 3 | 1 | 227420689 | 227432711 | H1C26819.1 | 963 | 964 |
| 3 | 1 | 227420689 | 227432729 | H1C26819.2 | 965 | 966 |
| 3 | 1 | 227420689 | 227443077 | H1C26819.3 | 967 | 968 |
| 3 | 1 | 227420689 | 227443078 | H1C26819.4 | 969 | 970 |
| 3 | 1 | 227420689 | 227443078 | H1C26819.5 | 971 | 972 |
| 3 | 1 | 227420689 | 227443215 | H1C26819.6 | 973 | 974 |
| 3 | 1 | 227420689 | 227443215 | H1C26819.7 | 975 | 976 |
| 3 | 1 | 227420689 | 227443215 | H1C26819.8 | 977 | 978 |
| 3 | 1 | 227421583 | 227443077 | H1C26819.9 | 979 | 980 |
| 3 | 1 | 227421583 | 227443215 | H1C26819.10 | 981 | 982 |
| 3 | 1 | 227438231 | 227443077 | H1C26819.11 | 983 | 984 |
| 3 | 1 | 227438231 | 227443215 | H1C26819.12 | 985 | 986 |
| 3 | 1 | 227439288 | 227443077 | H1C26819.13 | 987 | 988 |
| 3 | 1 | 227439288 | 227443215 | H1C26819.14 | 989 | 990 |
| 3 | 1 | 227442387 | 227482431 | H1C26823.1 | 991 | 992 |
| 3 | 1 | 227461339 | 227471213 | H1C26823.2 | 993 | 994 |
| 3 | 1 | 227461339 | 227483029 | H1C26823.3 | 995 | 996 |
| 3 | 1 | 227461339 | 227483029 | H1C26823.4 | 997 | 998 |
| 3 | 1 | 227461339 | 227483938 | H1C26823.5 | 999 | 1000 |
| 3 | 1 | 227461438 | 227462728 | H1C26826.1 | 1001 | 1002 |
| 4 | 1 | 235875905 | 236375937 | H1C27733.1 | 1003 | 1004 |
| 4 | 1 | 236118413 | 236397936 | H1C27733.3 | 1005 | 1006 |
| 4 | 1 | 236167524 | 236193000 | H1C27733.4 | 1007 | 1008 |
| 4 | 1 | 236193226 | 236208407 | H1C27763.1 | 1009 | 1010 |
| 4 | 1 | 236195661 | 236197405 | H1C27763.2 | 1011 | 1012 |
| 4 | 1 | 236208887 | 236397870 | H1C27733.5 | 1013 | 1014 |
| 4 | 1 | 236387356 | 236389213 | H1C27790.1 | 1015 | 1016 |
| 4 | 1 | 236396635 | 236398955 | H1C27791.1 | 1017 | 1018 |
| 4 | 1 | 236404784 | 236406296 | H1C27794.1 | 1019 | 1020 |
| 4 | 1 | 236496864 | 236502601 | H1C27796.1 | 1021 | 1022 |
| 4 | 1 | 236498503 | 236502601 | H1C27797.1 | 1023 | 1024 |
| 4 | 1 | 236503688 | 236697267 | H1C27798.1 | 1025 | 1026 |
| 4 | 1 | 236581021 | 236964526 | H1C27798.2 | 1027 | 1028 |
| 5 | 2 | 20805416 | 20944487 | H2C1836.1 | 1029 | 1030 |
| 5 | 2 | 20806441 | 20944502 | H2C1836.2 | 1031 | 1032 |
| 5 | 2 | 20806614 | 20944502 | H2C1836.3 | 1033 | 1034 |
| 5 | 2 | 20910286 | 20944498 | H2C1836.4 | 1035 | 1036 |
| 5 | 2 | 20981158 | 20983048 | H2C1854.1 | 1037 | 1038 |
| 5 | 2 | 21135828 | 21178598 | H2C1857.1 | 1039 | 1040 |
| 5 | 2 | 21135948 | 21178598 | H2C1857.2 | 1041 | 1042 |
| 5 | 2 | 21139121 | 21141075 | H2C1859.1 | 1043 | 1044 |
| 5 | 2 | 21158697 | 21178598 | H2C1857.3 | 1045 | 1046 |
| 5 | 2 | 21258135 | 21277796 | H2C1876.1 | 1047 | 1048 |
| 5 | 2 | 21355698 | 22105483 | H2C1881.1 | 1049 | 1050 |
| 5 | 2 | 21355708 | 21398609 | H2C1881.2 | 1051 | 1052 |
| 6, 7 | 2 | 50057294 | 51167770 | H2C5527.1 | 1053 | 1054 |
| 6, 7 | 2 | 50057651 | 50112955 | H2C5527.2 | 1055 | 1056 |
| 6, 7 | 2 | 50057651 | 50155334 | H2C5527.3 | 1057 | 1058 |
| 6, 7 | 2 | 50057651 | 50158064 | H2C5527.4 | 1059 | 1060 |
| 6, 7 | 2 | 50057651 | 50486581 | H2C5527.5 | 1061 | 1062 |
| 6, 7 | 2 | 50057651 | 51167770 | H2C5527.6 | 1063 | 1064 |
| 7 | 2 | 50057651 | 51167770 | H2C5527.6 | 1063 | 1064 |
| 7 | 2 | 50120411 | 50122147 | H2C5534.1 | 1065 | 1066 |
| 7 | 2 | 50337930 | 50795203 | H2C5527.7 | 1067 | 1068 |
| 7 | 2 | 50564517 | 50567361 | H2C5567.1 | 1069 | 1070 |
| 7 | 2 | 50755052 | 50758973 | H2C5527.8 | 1071 | 1072 |
| 7 | 2 | 50757594 | 50776353 | H2C5527.9 | 1073 | 1074 |
| 7 | 2 | 50770063 | 50771786 | H2C5588.1 | 1075 | 1076 |
| 7 | 2 | 51055611 | 51171325 | H2C5527.10 | 1077 | 1078 |
| 7 | 2 | 51058251 | 51061479 | H2C5527.11 | 1079 | 1080 |
| 7 | 2 | 51061310 | 51166570 | H2C5527.12 | 1081 | 1082 |
| 7 | 2 | 51061443 | 51166800 | H2C5527.13 | 1083 | 1084 |
| 7 | 2 | 51171389 | 52546706 | H2C5643.1 | 1085 | 1086 |
| 7 | 2 | 51340732 | 51360553 | H2C5646.1 | 1087 | 1088 |
| 8 | 2 | 121690392 | 121759005 | H2C12975.1 | 1089 | 1090 |
| 8 | 2 | 121692109 | 121759005 | H2C12975.2 | 1091 | 1092 |
| 8 | 2 | 121694762 | 121759008 | H2C12975.3 | 1093 | 1094 |
| 8 | 2 | 121694762 | 121759008 | H2C12975.4 | 1095 | 1096 |
| 8 | 2 | 121811582 | 121813739 | H2C12995.1 | 1097 | 1098 |
| 8 | 2 | 121811582 | 121852446 | H2C12996.1 | 1099 | 1100 |
| 8 | 2 | 121811582 | 121903910 | H2C12996.2 | 1101 | 1102 |
| 8 | 2 | 121811582 | 122123393 | H2C12996.3 | 1103 | 1104 |
| 8 | 2 | 121811582 | 122123393 | H2C12996.4 | 1105 | 1106 |
| 8 | 2 | 121814491 | 121839091 | H2C12996.5 | 1107 | 1108 |
| 8 | 2 | 121837937 | 121861882 | H2C12996.6 | 1109 | 1110 |
| 8 | 2 | 121933223 | 121935001 | H2C12996.7 | 1111 | 1112 |
| 8 | 2 | 121968853 | 121969718 | H2C13035.1 | 1113 | 1114 |
| 8 | 2 | 122064690 | 122071682 | H2C13053.1 | 1115 | 1116 |
| 8 | 2 | 122123455 | 122124968 | H2C13061.1 | 1117 | 1118 |
| 8 | 2 | 122123455 | 122125344 | H2C13061.2 | 1119 | 1120 |
| 8 | 2 | 122123475 | 122124968 | H2C13061.3 | 1121 | 1122 |
| 8 | 2 | 122123475 | 122125344 | H2C13061.4 | 1123 | 1124 |
| 8 | 2 | 122123475 | 122202680 | H2C13061.5 | 1125 | 1126 |
| 8 | 2 | 122123603 | 122124968 | H2C13061.6 | 1127 | 1128 |
| 8 | 2 | 122123603 | 122125344 | H2C13061.7 | 1129 | 1130 |
| 8 | 2 | 122123794 | 122124968 | H2C13061.8 | 1131 | 1132 |
| 8 | 2 | 122123794 | 122125344 | H2C13061.9 | 1133 | 1134 |
| 8 | 2 | 122200739 | 122202680 | H2C13071.1 | 1135 | 1136 |
| 8 | 2 | 122200739 | 122202680 | H2C13071.2 | 1137 | 1138 |
| 8 | 2 | 122200739 | 122209718 | H2C13071.3 | 1139 | 1140 |
| 8 | 2 | 122200739 | 122210720 | H2C13071.4 | 1141 | 1142 |
| 8 | 2 | 122200782 | 122202680 | H2C13071.5 | 1143 | 1144 |
| 8 | 2 | 122200782 | 122202680 | H2C13071.6 | 1145 | 1146 |
| 8 | 2 | 122200782 | 122209718 | H2C13071.7 | 1147 | 1148 |
| 8 | 2 | 122200782 | 122210720 | H2C13071.8 | 1149 | 1150 |
| 8 | 2 | 122200850 | 122202680 | H2C13071.10 | 1151 | 1152 |
| 8 | 2 | 122200850 | 122202680 | H2C13071.9 | 1153 | 1154 |
| 8 | 2 | 122200850 | 122209718 | H2C13071.11 | 1155 | 1156 |
| 8 | 2 | 122200850 | 122210720 | H2C13071.12 | 1157 | 1158 |
| 8 | 2 | 122201477 | 122202680 | H2C13071.13 | 1159 | 1160 |
| 8 | 2 | 122201477 | 122202680 | H2C13071.14 | 1161 | 1162 |
| 8 | 2 | 122201477 | 122204995 | H2C13071.15 | 1163 | 1164 |
| 8 | 2 | 122201477 | 122209718 | H2C13071.16 | 1165 | 1166 |
| 8 | 2 | 122201477 | 122210720 | H2C13071.17 | 1167 | 1168 |
| 8 | 2 | 122229338 | 122239472 | H2C13076.1 | 1169 | 1170 |
| 8 | 2 | 122229338 | 122239629 | H2C13076.2 | 1171 | 1172 |
| 8 | 2 | 122229338 | 122241097 | H2C13076.3 | 1173 | 1174 |
| 8 | 2 | 122229338 | 122241659 | H2C13076.4 | 1175 | 1176 |
| 8 | 2 | 122229350 | 122239472 | H2C13076.5 | 1177 | 1178 |
| 8 | 2 | 122229350 | 122239629 | H2C13076.6 | 1179 | 1180 |
| 8 | 2 | 122229350 | 122241097 | H2C13076.7 | 1181 | 1182 |
| 8 | 2 | 122229350 | 122241656 | H2C13076.8 | 1183 | 1184 |
| 8 | 2 | 122229453 | 122239472 | H2C13076.9 | 1185 | 1186 |
| 8 | 2 | 122229453 | 122239629 | H2C13076.10 | 1187 | 1188 |
| 8 | 2 | 122229453 | 122241097 | H2C13076.11 | 1189 | 1190 |
| 8 | 2 | 122229453 | 122241656 | H2C13076.12 | 1191 | 1192 |
| 8 | 2 | 122229465 | 122239472 | H2C13076.13 | 1193 | 1194 |
| 8 | 2 | 122229465 | 122239472 | H2C13076.14 | 1195 | 1196 |
| 8 | 2 | 122229465 | 122239629 | H2C13076.15 | 1197 | 1198 |
| 8 | 2 | 122229465 | 122239629 | H2C13076.16 | 1199 | 1200 |
| 8 | 2 | 122229465 | 122241097 | H2C13076.17 | 1201 | 1202 |
| 8 | 2 | 122229465 | 122241097 | H2C13076.18 | 1203 | 1204 |
| 8 | 2 | 122229465 | 122241097 | H2C13076.19 | 1205 | 1206 |
| 8 | 2 | 122229465 | 122241656 | H2C13076.20 | 1207 | 1208 |
| 8 | 2 | 122229465 | 122241656 | H2C13076.21 | 1209 | 1210 |
| 8 | 2 | 122229465 | 122241656 | H2C13076.22 | 1211 | 1212 |
| 8 | 2 | 122239625 | 122241097 | H2C13079.1 | 1213 | 1214 |
| 8 | 2 | 122239625 | 122241656 | H2C13079.2 | 1215 | 1216 |
| 8 | 2 | 122264249 | 122270508 | H2C13082.1 | 1217 | 1218 |
| 8 | 2 | 122264268 | 122268846 | H2C13082.2 | 1219 | 1220 |
| 8 | 2 | 122264274 | 122270162 | H2C13082.3 | 1221 | 1222 |
| 8 | 2 | 122266993 | 122268846 | H2C13082.4 | 1223 | 1224 |
| 8 | 2 | 122266995 | 122274600 | H2C13082.5 | 1225 | 1226 |
| 8 | 2 | 122348249 | 122681062 | H2C13084.1 | 1227 | 1228 |
| 9 | 2 | 127499621 | 127507679 | H2C13211.1 | 1229 | 1230 |
| 9 | 2 | 127521828 | 127530297 | H2C13215.1 | 1231 | 1232 |
| 9 | 2 | 127521828 | 127580758 | H2C13215.2 | 1233 | 1234 |
| 9 | 2 | 127521828 | 127580808 | H2C13215.3 | 1235 | 1236 |
| 9 | 2 | 127521828 | 127581149 | H2C13215.4 | 1237 | 1238 |
| 9 | 2 | 127521828 | 127581154 | H2C13215.10 | 1239 | 1240 |
| 9 | 2 | 127521828 | 127581154 | H2C13215.11 | 1241 | 1242 |
| 9 | 2 | 127521828 | 127581154 | H2C13215.12 | 1243 | 1244 |
| 9 | 2 | 127521828 | 127581154 | H2C13215.13 | 1245 | 1246 |
| 9 | 2 | 127521828 | 127581154 | H2C13215.14 | 1247 | 1248 |
| 9 | 2 | 127521828 | 127581154 | H2C13215.15 | 1249 | 1250 |
| 9 | 2 | 127521828 | 127581154 | H2C13215.5 | 1251 | 1252 |
| 9 | 2 | 127521828 | 127581154 | H2C13215.6 | 1253 | 1254 |
| 9 | 2 | 127521828 | 127581154 | H2C13215.7 | 1255 | 1256 |
| 9 | 2 | 127521828 | 127581154 | H2C13215.8 | 1257 | 1258 |
| 9 | 2 | 127521828 | 127581154 | H2C13215.9 | 1259 | 1260 |
| 9 | 2 | 127581502 | 127583078 | H2C13224.1 | 1261 | 1262 |
| 9 | 2 | 127616699 | 127620765 | H2C13226.1 | 1263 | 1264 |
| 9 | 2 | 127657641 | 127679573 | H2C13227.1 | 1265 | 1266 |
| 9 | 2 | 127731095 | 127737249 | H2C13231.1 | 1267 | 1268 |
| 9 | 2 | 127731095 | 127763633 | H2C13231.2 | 1269 | 1270 |
| 9 | 2 | 127731095 | 127766533 | H2C13231.3 | 1271 | 1272 |
| 9 | 2 | 127731095 | 127767887 | H2C13231.4 | 1273 | 1274 |
| 9 | 2 | 127731095 | 127767900 | H2C13231.5 | 1275 | 1276 |
| 9 | 2 | 127731095 | 127767982 | H2C13231.6 | 1277 | 1278 |
| 9 | 2 | 127731096 | 127763859 | H2C13232.1 | 1279 | 1280 |
| 9 | 2 | 127763141 | 127767808 | H2C13231.7 | 1281 | 1282 |
| 9 | 2 | 127763195 | 127764831 | H2C13231.8 | 1283 | 1284 |
| 9 | 2 | 127763341 | 127767951 | H2C13231.9 | 1285 | 1286 |
| 9 | 2 | 127764222 | 127765594 | H2C13243.1 | 1287 | 1288 |
| 9 | 2 | 127766274 | 127767973 | H2C13231.10 | 1289 | 1290 |
| 9 | 2 | 127772473 | 127817279 | H2C13245.1 | 1291 | 1292 |
| 9 | 2 | 127774196 | 127817279 | H2C13245.2 | 1293 | 1294 |
| 9 | 2 | 127778143 | 127817279 | H2C13245.3 | 1295 | 1296 |
| 9 | 2 | 127780467 | 127817279 | H2C13245.4 | 1297 | 1298 |
| 9 | 2 | 127803724 | 127804531 | H2C13245.5 | 1299 | 1300 |
| 9 | 2 | 127861270 | 127863217 | H2C13269.1 | 1301 | 1302 |
| 9 | 2 | 127862066 | 127863217 | H2C13269.2 | 1303 | 1304 |
| 9 | 2 | 127862090 | 127863211 | H2C13269.3 | 1305 | 1306 |
| 9 | 2 | 127862939 | 127874385 | H2C13273.1 | 1307 | 1308 |
| 9 | 2 | 127884892 | 127886096 | H2C13277.1 | 1309 | 1310 |
| 9 | 2 | 127892232 | 127903051 | H2C13280.1 | 1311 | 1312 |
| 9 | 2 | 127895087 | 127897033 | H2C13280.2 | 1313 | 1314 |
| 9 | 2 | 127897094 | 127903051 | H2C13280.3 | 1315 | 1316 |
| 9 | 2 | 127911723 | 127912795 | H2C13286.1 | 1317 | 1318 |
| 9 | 2 | 127943407 | 127944094 | H2C13285.1 | 1319 | 1320 |
| 9 | 2 | 127954607 | 127959773 | H2C13285.2 | 1321 | 1322 |
| 9 | 2 | 127954607 | 127963536 | H2C13285.3 | 1323 | 1324 |
| 9 | 2 | 127954607 | 128000248 | H2C13285.4 | 1325 | 1326 |
| 9 | 2 | 127954607 | 128000293 | H2C13285.5 | 1327 | 1328 |
| 9 | 2 | 127954607 | 128000295 | H2C13285.6 | 1329 | 1330 |
| 9 | 2 | 127976560 | 127979513 | H2C13285.7 | 1331 | 1332 |
| 9 | 2 | 127978489 | 127979336 | H2C13296.1 | 1333 | 1334 |
| 9 | 2 | 127978532 | 128000265 | H2C13285.8 | 1335 | 1336 |
| 9 | 2 | 127978598 | 128000274 | H2C13285.9 | 1337 | 1338 |
| 9 | 2 | 127978598 | 128000295 | H2C13285.10 | 1339 | 1340 |
| 9 | 2 | 127978598 | 128000596 | H2C13285.11 | 1341 | 1342 |
| 9 | 2 | 128009607 | 128088452 | H2C13305.1 | 1343 | 1344 |
| 9 | 2 | 128033849 | 128044767 | H2C13308.1 | 1345 | 1346 |
| 10 | 2 | 161990055 | 162093736 | H2C16344.1 | 1347 | 1348 |
| 10 | 2 | 162079113 | 162093735 | H2C16344.3 | 1349 | 1350 |
| 10 | 2 | 162089545 | 162093735 | H2C16344.4 | 1351 | 1352 |
| 10 | 2 | 162098126 | 162107183 | H2C16365.1 | 1353 | 1354 |
| 10 | 2 | 162104676 | 162105153 | H2C16368.1 | 1355 | 1356 |
| 10 | 2 | 162104676 | 162105595 | H2C16368.2 | 1357 | 1358 |
| 10 | 2 | 162306353 | 162667292 | H2C16379.1 | 1359 | 1360 |
| 10 | 2 | 162674257 | 162675316 | H2C16392.1 | 1361 | 1362 |
| 10 | 2 | 162674257 | 162675402 | H2C16392.2 | 1363 | 1364 |
| 10 | 2 | 162674257 | 162756559 | H2C16393.1 | 1365 | 1366 |
| 10 | 2 | 162755272 | 162757352 | H2C16404.1 | 1367 | 1368 |
| 10 | 2 | 162775445 | 162776881 | H2C16406.1 | 1369 | 1370 |
| 10 | 2 | 162796457 | 162854748 | H2C16409.1 | 1371 | 1372 |
| 10 | 2 | 162824885 | 162826597 | H2C16410.1 | 1373 | 1374 |
| 10 | 2 | 162824891 | 162826733 | H2C16410.2 | 1375 | 1376 |
| 10 | 2 | 162824891 | 162831611 | H2C16410.3 | 1377 | 1378 |
| 10 | 2 | 162824891 | 162834217 | H2C16410.4 | 1379 | 1380 |
| 10 | 2 | 162824891 | 162834233 | H2C16410.5 | 1381 | 1382 |
| 10 | 2 | 162824891 | 162834254 | H2C16410.6 | 1383 | 1384 |
| 10 | 2 | 162824891 | 162834421 | H2C16410.7 | 1385 | 1386 |
| 10 | 2 | 162825167 | 162831611 | H2C16410.8 | 1387 | 1388 |
| 10 | 2 | 162825167 | 162834217 | H2C16410.9 | 1389 | 1390 |
| 10 | 2 | 162825167 | 162834233 | H2C16410.10 | 1391 | 1392 |
| 10 | 2 | 162825167 | 162834421 | H2C16410.11 | 1393 | 1394 |
| 10 | 2 | 162825187 | 162826155 | H2C16410.12 | 1395 | — |
| 10 | 2 | 162825187 | 162826733 | H2C16410.13 | 1396 | 1397 |
| 10 | 2 | 162825187 | 162834254 | H2C16410.14 | 1398 | 1399 |
| 10 | 2 | 162826042 | 162829410 | H2C16410.15 | 1400 | 1401 |
| 10 | 2 | 162826799 | 162834254 | H2C16410.16 | 1402 | 1403 |
| 10 | 2 | 162827636 | 162831206 | H2C16410.17 | 1404 | 1405 |
| 10 | 2 | 162852700 | 162855078 | H2C16415.1 | 1406 | 1407 |
| 10 | 2 | 162852700 | 162858686 | H2C16415.2 | 1408 | 1409 |
| 10 | 2 | 162852700 | 162925475 | H2C16415.3 | 1410 | 1411 |
| 10 | 2 | 162852700 | 162925552 | H2C16415.4 | 1412 | 1413 |
| 10 | 2 | 162871199 | 162874055 | H2C16415.5 | 1414 | 1415 |
| 10 | 2 | 162949090 | 163000927 | H2C16427.1 | 1416 | 1417 |
| 10 | 2 | 162949096 | 162950154 | H2C16427.2 | 1418 | 1419 |
| 10 | 2 | 162963470 | 163000927 | H2C16427.3 | 1420 | 1421 |
| 10 | 2 | 162992165 | 163000927 | H2C16427.4 | 1422 | 1423 |
| 10 | 2 | 163000855 | 163038851 | H2C16433.1 | 1424 | 1425 |
| 10 | 2 | 163000898 | 163038825 | H2C16433.2 | 1426 | 1427 |
| 10 | 2 | 163001050 | 163043145 | H2C16433.3 | 1428 | 1429 |
| 10 | 2 | 163001050 | 163043439 | H2C16433.4 | 1430 | 1431 |
| 11 | 2 | 198494931 | 198838635 | H2C19961.1 | 1432 | 1433 |
| 11 | 2 | 198656803 | 198838635 | H2C19961.2 | 1434 | 1435 |
| 11 | 2 | 198836837 | 198837684 | H2C19961.3 | 1436 | 1437 |
| 11 | 2 | 198878033 | 198881753 | H2C20006.1 | 1438 | 1439 |
| 11 | 2 | 198881193 | 198881753 | H2C20007.1 | 1440 | — |
| 11 | 2 | 198881506 | 198884102 | H2C20008.1 | 1441 | 1442 |
| 11 | 2 | 198989592 | 199064779 | H2C20010.1 | 1443 | 1444 |
| 11 | 2 | 199037047 | 199262811 | H2C20012.1 | 1445 | 1446 |
| 11 | 2 | 199064704 | 199065327 | H2C20013.1 | 1447 | 1448 |
| 12 | 3 | 6507165 | 6753421 | H3C450.1 | 1449 | 1450 |
| 12 | 3 | 6648540 | 6661580 | H3C463.1 | 1451 | 1452 |
| 12 | 3 | 6648541 | 6681593 | H3C463.2 | 1453 | 1454 |
| 12 | 3 | 6648541 | 6691303 | H3C463.3 | 1455 | 1456 |
| 12 | 3 | 6649052 | 6681593 | H3C463.4 | 1457 | 1458 |
| 12 | 3 | 6649052 | 6807825 | H3C463.5 | 1459 | 1460 |
| 12 | 3 | 6697780 | 6741233 | H3C450.2 | 1461 | 1462 |
| 12 | 3 | 6711409 | 6714557 | H3C450.3 | 1463 | 1464 |
| 12 | 3 | 6738001 | 6807792 | H3C463.6 | 1465 | 1466 |
| 12 | 3 | 6747859 | 6822125 | H3C463.7 | 1467 | 1468 |
| 12 | 3 | 6749026 | 6756153 | H3C463.8 | 1469 | 1470 |
| 12 | 3 | 6756224 | 6822114 | H3C463.9 | 1471 | 1472 |
| 12 | 3 | 6757054 | 6759374 | H3C476.1 | 1473 | 1474 |
| 12 | 3 | 6805622 | 6807830 | H3C463.10 | 1475 | 1476 |
| 12 | 3 | 6877837 | 7758219 | H3C482.1 | 1477 | 1478 |
| 12 | 3 | 6877926 | 7711148 | H3C482.2 | 1479 | 1480 |
| 12 | 3 | 6877926 | 7758219 | H3C482.3 | 1481 | 1482 |
| 12 | 3 | 6879566 | 7323340 | H3C482.4 | 1483 | 1484 |
| 12 | 3 | 6909323 | 6910709 | H3C487.1 | 1485 | 1486 |
| 12 | 3 | 7468467 | 7653128 | H3C482.5 | 1487 | 1488 |
| 12 | 3 | 7707466 | 7711148 | H3C482.6 | 1489 | 1490 |
| 12 | 3 | 7709832 | 7711152 | H3C482.7 | 1491 | 1492 |
| 12 | 3 | 7751364 | 7751971 | H3C511.1 | 1493 | 1494 |
| 12 | 3 | 7969491 | 8032994 | H3C524.1 | 1495 | 1496 |
| 13 | 3 | 32701683 | 32790208 | H3C3250.1 | 1497 | 1498 |
| 13 | 3 | 32701683 | 32790208 | H3C3250.2 | 1499 | 1500 |
| 13 | 3 | 32701683 | 32790371 | H3C3250.3 | 1501 | 1502 |
| 13 | 3 | 32769496 | 32790208 | H3C3250.5 | 1503 | 1504 |
| 13 | 3 | 32769496 | 32790371 | H3C3250.6 | 1505 | 1506 |
| 13 | 3 | 32769635 | 32777990 | H3C3257.1 | 1507 | 1508 |
| 13 | 3 | 32780716 | 32790208 | H3C3250.7 | 1509 | 1510 |
| 13 | 3 | 32780716 | 32790371 | H3C3250.8 | 1511 | 1512 |
| 13 | 3 | 32780808 | 32790370 | H3C3263.1 | 1513 | 1514 |
| 13 | 3 | 32781038 | 32790208 | H3C3250.9 | 1515 | 1516 |
| 13 | 3 | 32781038 | 32790371 | H3C3250.10 | 1517 | 1518 |
| 13 | 3 | 32783184 | 32783942 | H3C3265.1 | 1519 | 1520 |
| 13 | 3 | 32786036 | 32790208 | H3C3250.11 | 1521 | 1522 |
| 13 | 3 | 32786036 | 32790370 | H3C3250.12 | 1523 | 1524 |
| 13 | 3 | 32788297 | 32790056 | H3C3269.1 | 1525 | 1526 |
| 13 | 3 | 32797820 | 32802664 | H3C3273.1 | 1527 | 1528 |
| 13 | 3 | 32832844 | 32833862 | H3C3276.1 | 1529 | 1530 |
| 13 | 3 | 32858710 | 32859454 | H3C3278.1 | 1531 | 1532 |
| 13 | 3 | 32902381 | 32908307 | H3C3284.1 | 1533 | 1534 |
| 13 | 3 | 32968043 | 32971404 | H3C3289.1 | 1535 | 1536 |
| 13 | 3 | 32977606 | 32991184 | H3C3292.1 | 1537 | 1538 |
| 13 | 3 | 33013109 | 33113685 | H3C3295.1 | 1539 | 1540 |
| 13 | 3 | 33013109 | 33113725 | H3C3295.2 | 1541 | 1542 |
| 13 | 3 | 33013222 | 33113685 | H3C3295.3 | 1543 | 1544 |
| 13 | 3 | 33013222 | 33113725 | H3C3295.4 | 1545 | 1546 |
| 13 | 3 | 33047194 | 33051082 | H3C3295.5 | 1547 | 1548 |
| 13 | 3 | 33061808 | 33074768 | H3C3295.6 | 1549 | 1550 |
| 13 | 3 | 33068471 | 33113725 | H3C3295.7 | 1551 | 1552 |
| 13 | 3 | 33106912 | 33108547 | H3C3314.1 | 1553 | 1554 |
| 13 | 3 | 33106912 | 33113297 | H3C3295.8 | 1555 | 1556 |
| 13 | 3 | 33106912 | 33113319 | H3C3295.9 | 1557 | 1558 |
| 13 | 3 | 33123250 | 33125865 | H3C3315.1 | 1559 | 1560 |
| 13 | 3 | 33130452 | 33137102 | H3C3316.1 | 1561 | 1562 |
| 13 | 3 | 33130452 | 33159686 | H3C3316.2 | 1563 | 1564 |
| 13 | 3 | 33130452 | 33159686 | H3C3316.3 | 1565 | 1566 |
| 13 | 3 | 33130452 | 33159686 | H3C3316.4 | 1567 | 1568 |
| 13 | 3 | 33130452 | 33160003 | H3C3316.5 | 1569 | 1570 |
| 13 | 3 | 33130452 | 33160003 | H3C3316.6 | 1571 | 1572 |
| 13 | 3 | 33130452 | 33160003 | H3C3316.7 | 1573 | 1574 |
| 13 | 3 | 33130452 | 33160475 | H3C3316.10 | 1575 | 1576 |
| 13 | 3 | 33130452 | 33160475 | H3C3316.8 | 1577 | 1578 |
| 13 | 3 | 33130452 | 33160475 | H3C3316.9 | 1579 | 1580 |
| 13 | 3 | 33130517 | 33161533 | H3C3317.1 | 1581 | 1582 |
| 13 | 3 | 33151243 | 33161533 | H3C3322.1 | 1583 | 1584 |
| 13 | 3 | 33158888 | 33159662 | H3C3324.1 | 1585 | 1586 |
| 13 | 3 | 33158888 | 33159662 | H3C3325.1 | 1587 | 1588 |
| 13 | 3 | 33158888 | 33160475 | H3C3323.1 | 1589 | 1590 |
| 13 | 3 | 33158888 | 33160475 | H3C3324.2 | 1591 | 1592 |
| 13 | 3 | 33158888 | 33161533 | H3C3326.1 | 1593 | 1594 |
| 13 | 3 | 33158888 | 33164269 | H3C3326.2 | 1595 | 1596 |
| 13 | 3 | 33160599 | 33164266 | H3C3328.1 | 1597 | 1598 |
| 13 | 3 | 33166537 | 33236511 | H3C3331.1 | 1599 | 1600 |
| 13 | 3 | 33293934 | 33403131 | H3C3334.1 | 1601 | 1602 |
| 13 | 3 | 33293934 | 33403760 | H3C3334.2 | 1603 | 1604 |
| 13 | 3 | 33294587 | 33295381 | H3C3335.1 | 1605 | 1606 |
| 13 | 3 | 33328570 | 33348128 | H3C3336.1 | 1607 | 1608 |
| 14 | 3 | 39826154 | 40276813 | H3C4013.1 | 1609 | 1610 |
| 14 | 3 | 39826154 | 40276813 | H3C4013.2 | 1611 | 1612 |
| 14 | 3 | 39924391 | 39928519 | H3C4017.1 | 1613 | 1614 |
| 14 | 3 | 40087615 | 40276813 | H3C4013.3 | 1615 | 1616 |
| 14 | 3 | 40121870 | 40316587 | H3C4035.1 | 1617 | 1618 |
| 14 | 3 | 40183419 | 40208292 | H3C4013.4 | 1619 | 1620 |
| 14 | 3 | 40189636 | 40326422 | H3C4038.1 | 1621 | 1622 |
| 14 | 3 | 40242099 | 40325986 | H3C4038.2 | 1623 | 1624 |
| 14 | 3 | 40260923 | 40325973 | H3C4038.3 | 1625 | 1626 |
| 14 | 3 | 40305621 | 40326422 | H3C4038.4 | 1627 | 1628 |
| 14 | 3 | 40326178 | 40328894 | H3C4055.1 | 1629 | 1630 |
| 14 | 3 | 40326178 | 40328919 | H3C4055.2 | 1631 | 1632 |
| 14 | 3 | 40326185 | 40328894 | H3C4055.3 | 1633 | 1634 |
| 14 | 3 | 40327092 | 40328155 | H3C4057.1 | 1635 | 1636 |
| 14 | 3 | 40403676 | 40445121 | H3C4063.1 | 1637 | 1638 |
| 14 | 3 | 40407445 | 40469800 | H3C4065.1 | 1639 | 1640 |
| 14 | 3 | 40408205 | 40469675 | H3C4065.2 | 1641 | 1642 |
| 14 | 3 | 40462026 | 40469824 | H3C4065.3 | 1643 | 1644 |
| 14 | 3 | 40473818 | 40478867 | H3C4077.1 | 1645 | 1646 |
| 14 | 3 | 40476386 | 40477767 | H3C4080.1 | 1647 | 1648 |
| 14 | 3 | 40477864 | 40478778 | H3C4077.2 | 1649 | 1650 |
| 14 | 3 | 40477974 | 40478778 | H3C4077.3 | 1651 | 1652 |
| 14 | 3 | 40477974 | 40478867 | H3C4077.4 | 1653 | 1654 |
| 14 | 3 | 40478101 | 40478778 | H3C4077.5 | 1655 | 1656 |
| 14 | 3 | 40478101 | 40478867 | H3C4077.6 | 1657 | 1658 |
| 14 | 3 | 40478444 | 40478778 | H3C4082.1 | 1659 | 1660 |
| 14 | 3 | 40478444 | 40478863 | H3C4082.2 | 1661 | 1662 |
| 14 | 3 | 40478870 | 40483649 | H3C4083.1 | 1663 | 1664 |
| 14 | 3 | 40493640 | 40504949 | H3C4086.1 | 1665 | 1666 |
| 14 | 3 | 40498393 | 40523951 | H3C4087.1 | 1667 | 1668 |
| 14 | 3 | 40498397 | 40503589 | H3C4086.2 | 1669 | 1670 |
| 14 | 3 | 40505070 | 40506732 | H3C4088.1 | 1671 | 1672 |
| 14 | 3 | 40507326 | 40508017 | H3C4089.1 | 1673 | 1674 |
| 14 | 3 | 40522486 | 40534051 | H3C4087.2 | 1675 | 1676 |
| 14 | 3 | 40522534 | 40534201 | H3C4087.3 | 1677 | 1678 |
| 14 | 3 | 40541372 | 40550438 | H3C4094.1 | 1679 | 1680 |
| 14 | 3 | 40541392 | 40549344 | H3C4094.2 | 1681 | 1682 |
| 14 | 3 | 40541392 | 40549344 | H3C4094.3 | 1683 | 1684 |
| 14 | 3 | 40541392 | 40556021 | H3C4094.4 | 1685 | 1686 |
| 14 | 3 | 40541392 | 40591182 | H3C4094.5 | 1687 | 1688 |
| 14 | 3 | 40541500 | 40549344 | H3C4094.6 | 1689 | 1690 |
| 14 | 3 | 40601956 | 40632304 | H3C4106.1 | 1691 | 1692 |
| 14 | 3 | 40619627 | 40621922 | H3C4107.1 | 1693 | 1694 |
| 15 | 4 | 77006937 | 77079451 | H4C6331.1 | 1695 | 1696 |
| 15 | 4 | 77006937 | 77092543 | H4C6331.2 | 1697 | 1698 |
| 15 | 4 | 77006937 | 77092620 | H4C6331.3 | 1699 | 1700 |
| 15 | 4 | 77006937 | 77092620 | H4C6331.4 | 1701 | 1702 |
| 15 | 4 | 77101353 | 77103878 | H4C6353.1 | 1703 | 1704 |
| 15 | 4 | 77102701 | 77103878 | H4C6353.2 | 1705 | 1706 |
| 15 | 4 | 77137568 | 77138968 | H4C6355.1 | 1707 | 1708 |
| 15 | 4 | 77138195 | 77148180 | H4C6356.1 | 1709 | 1710 |
| 15 | 4 | 77138197 | 77144325 | H4C6357.1 | 1711 | 1712 |
| 15 | 4 | 77138197 | 77180903 | H4C6358.1 | 1713 | 1714 |
| 15 | 4 | 77138197 | 77180903 | H4C6358.2 | 1715 | 1716 |
| 15 | 4 | 77138197 | 77180903 | H4C6358.3 | 1717 | 1718 |
| 15 | 4 | 77161280 | 77161849 | H4C6358.4 | 1719 | 1720 |
| 15 | 4 | 77166632 | 77168374 | H4C6358.5 | 1721 | 1722 |
| 15 | 4 | 77189594 | 77199073 | H4C6366.1 | 1723 | 1724 |
| 15 | 4 | 77195814 | 77219345 | H4C6365.1 | 1725 | 1726 |
| 15 | 4 | 77228243 | 77269292 | H4C6374.1 | 1727 | 1728 |
| 15 | 4 | 77251767 | 77252730 | H4C6383.1 | 1729 | 1730 |
| 15 | 4 | 77259121 | 77284271 | H4C6387.1 | 1731 | 1732 |
| 15 | 4 | 77279674 | 77285841 | H4C6391.1 | 1733 | 1734 |
| 15 | 4 | 77279674 | 77289473 | H4C6391.2 | 1735 | 1736 |
| 15 | 4 | 77279801 | 77285841 | H4C6391.3 | 1737 | 1738 |
| 15 | 4 | 77279801 | 77289473 | H4C6391.4 | 1739 | 1740 |
| 15 | 4 | 77289531 | 77391124 | H4C6393.1 | 1741 | 1742 |
| 15 | 4 | 77299447 | 77301868 | H4C6395.1 | 1743 | 1744 |
| 15 | 4 | 77312020 | 77314529 | H4C6397.1 | 1745 | 1746 |
| 15 | 4 | 77312020 | 77319747 | H4C6397.2 | 1747 | 1748 |
| 15 | 4 | 77312129 | 77319747 | H4C6397.3 | 1749 | 1750 |
| 15 | 4 | 77353024 | 77391071 | H4C6393.2 | 1751 | 1752 |
| 15 | 4 | 77353024 | 77391124 | H4C6393.3 | 1753 | 1754 |
| 15 | 4 | 77353041 | 77357441 | H4C6393.4 | 1755 | 1756 |
| 15 | 4 | 77353079 | 77391124 | H4C6393.5 | 1757 | 1758 |
| 15 | 4 | 77385547 | 77391124 | H4C6393.6 | 1759 | 1760 |
| 15 | 4 | 77392865 | 77396107 | H4C6409.1 | 1761 | 1762 |
| 15 | 4 | 77392865 | 77396774 | H4C6409.2 | 1763 | 1764 |
| 15 | 4 | 77392865 | 77426834 | H4C6409.3 | 1765 | 1766 |
| 15 | 4 | 77392992 | 77396774 | H4C6409.4 | 1767 | 1768 |
| 15 | 4 | 77392992 | 77409744 | H4C6409.5 | 1769 | 1770 |
| 15 | 4 | 77392992 | 77426778 | H4C6409.6 | 1771 | 1772 |
| 15 | 4 | 77392992 | 77426834 | H4C6409.7 | 1773 | 1774 |
| 15 | 4 | 77393159 | 77426834 | H4C6409.8 | 1775 | 1776 |
| 15 | 4 | 77393200 | 77396107 | H4C6409.9 | 1777 | 1778 |
| 15 | 4 | 77393200 | 77396774 | H4C6409.10 | 1779 | 1780 |
| 15 | 4 | 77437059 | 77439737 | H4C6422.1 | 1781 | 1782 |
| 15 | 4 | 77437059 | 77492258 | H4C6423.1 | 1783 | 1784 |
| 15 | 4 | 77439785 | 77492258 | H4C6423.2 | 1785 | 1786 |
| 15 | 4 | 77440597 | 77441508 | H4C6425.1 | 1787 | 1788 |
| 15 | 4 | 77457228 | 77462054 | H4C6437.1 | 1789 | 1790 |
| 15 | 4 | 77457699 | 77492258 | H4C6423.3 | 1791 | 1792 |
| 15 | 4 | 77457920 | 77492258 | H4C6423.4 | 1793 | 1794 |
| 15 | 4 | 77461862 | 77462675 | H4C6439.1 | 1795 | 1796 |
| 15 | 4 | 77492371 | 77562112 | H4C6443.1 | 1797 | 1798 |
| 15 | 4 | 77530052 | 77589462 | H4C6443.2 | 1799 | 1800 |
| 15 | 4 | 77546991 | 77550268 | H4C6443.3 | 1801 | 1802 |
| 15 | 4 | 77557517 | 77562106 | H4C6443.4 | 1803 | 1804 |
| 16 | 4 | 100853158 | 100862526 | H4C8545.1 | 1805 | 1806 |
| 16 | 4 | 100853158 | 100902332 | H4C8545.2 | 1807 | 1808 |
| 16 | 4 | 100914864 | 100933921 | H4C8552.1 | 1809 | 1810 |
| 16 | 4 | 101095161 | 101139960 | H4C8556.1 | 1811 | 1812 |
| 16 | 4 | 101095161 | 101147036 | H4C8556.2 | 1813 | 1814 |
| 16 | 4 | 101095161 | 101147945 | H4C8556.3 | 1815 | 1816 |
| 16 | 4 | 101095161 | 101148522 | H4C8556.4 | 1817 | 1818 |
| 16 | 4 | 101095161 | 101148522 | H4C8556.5 | 1819 | 1820 |
| 16 | 4 | 101142598 | 101148522 | H4C8556.6 | 1821 | 1822 |
| 16 | 4 | 101156672 | 101172881 | H4C8566.1 | 1823 | 1824 |
| 16 | 4 | 101156672 | 101172881 | H4C8566.2 | 1825 | 1826 |
| 16 | 4 | 101156724 | 101172881 | H4C8566.3 | 1827 | 1828 |
| 16 | 4 | 101156724 | 101172881 | H4C8566.4 | 1829 | 1830 |
| 16 | 4 | 101159540 | 101172881 | H4C8566.5 | 1831 | 1832 |
| 16 | 4 | 101159540 | 101172881 | H4C8566.6 | 1833 | 1834 |
| 16 | 4 | 101174582 | 101177015 | H4C8571.1 | 1835 | 1836 |
| 16 | 4 | 101174582 | 101184927 | H4C8572.1 | 1837 | 1838 |
| 16 | 4 | 101174582 | 101205426 | H4C8572.2 | 1839 | 1840 |
| 16 | 4 | 101174582 | 101224923 | H4C8572.3 | 1841 | 1842 |
| 16 | 4 | 101174582 | 101224968 | H4C8572.4 | 1843 | 1844 |
| 16 | 4 | 101177881 | 101184927 | H4C8572.5 | 1845 | 1846 |
| 16 | 4 | 101178857 | 101224968 | H4C8572.6 | 1847 | 1848 |
| 16 | 4 | 101200817 | 101224968 | H4C8572.7 | 1849 | 1850 |
| 16 | 4 | 101201588 | 101224968 | H4C8572.8 | 1851 | 1852 |
| 16 | 4 | 101202408 | 101224921 | H4C8572.9 | 1853 | 1854 |
| 16 | 4 | 101226393 | 101228723 | H4C8588.1 | 1855 | 1856 |
| 16 | 4 | 101226408 | 101228094 | H4C8588.2 | 1857 | 1858 |
| 16 | 4 | 101226408 | 101228592 | H4C8588.3 | 1859 | 1860 |
| 16 | 4 | 101226408 | 101228634 | H4C8588.4 | 1861 | 1862 |
| 16 | 4 | 101226408 | 101228634 | H4C8588.5 | 1863 | 1864 |
| 16 | 4 | 101226408 | 101228690 | H4C8588.6 | 1865 | 1866 |
| 16 | 4 | 101226408 | 101228716 | H4C8588.7 | 1867 | 1868 |
| 16 | 4 | 101226419 | 101228690 | H4C8588.8 | 1869 | 1870 |
| 16 | 4 | 101227201 | 101228690 | H4C8588.9 | 1871 | 1872 |
| 16 | 4 | 101228340 | 101244660 | H4C8590.1 | 1873 | 1874 |
| 16 | 4 | 101228828 | 101316148 | H4C8593.1 | 1875 | 1876 |
| 17 | 4 | 126595171 | 126601376 | H4C10553.1 | 1877 | 1878 |
| 17 | 4 | 126731474 | 126771691 | H4C10567.1 | 1879 | 1880 |
| 17 | 4 | 126768932 | 126771686 | H4C10570.1 | 1881 | 1882 |
| 17 | 4 | 126955477 | 127031553 | H4C10572.1 | 1883 | 1884 |
| 18 | 4 | 143305522 | 143752172 | H4C11432.1 | 1885 | 1886 |
| 18 | 4 | 143305522 | 143753027 | H4C11432.2 | 1887 | 1888 |
| 18 | 4 | 143305522 | 143753166 | H4C11432.3 | 1889 | 1890 |
| 18 | 4 | 143305522 | 143793029 | H4C11432.4 | 1891 | 1892 |
| 18 | 4 | 143305522 | 143839425 | H4C11432.5 | 1893 | 1894 |
| 18 | 4 | 143305522 | 144004658 | H4C11432.6 | 1895 | 1896 |
| 18 | 4 | 143307483 | 143752172 | H4C11432.7 | 1897 | 1898 |
| 18 | 4 | 143307483 | 143753027 | H4C11432.8 | 1899 | 1900 |
| 18 | 4 | 143307483 | 143753166 | H4C11432.9 | 1901 | 1902 |
| 18 | 4 | 143307483 | 143793029 | H4C11432.10 | 1903 | 1904 |
| 18 | 4 | 143307483 | 143839425 | H4C11432.11 | 1905 | 1906 |
| 18 | 4 | 143307483 | 144004658 | H4C11432.12 | 1907 | 1908 |
| 18 | 4 | 143471856 | 144004661 | H4C11432.13 | 1909 | 1910 |
| 18 | 4 | 143676169 | 143684210 | H4C11432.14 | 1911 | 1912 |
| 18 | 4 | 143705240 | 143710242 | H4C11432.15 | 1913 | 1914 |
| 18 | 4 | 143705240 | 143753166 | H4C11432.16 | 1915 | 1916 |
| 18 | 4 | 143793203 | 143797745 | H4C11496.1 | 1917 | 1918 |
| 18 | 4 | 143844774 | 143939708 | H4C11503.1 | 1919 | 1920 |
| 18 | 4 | 143933269 | 144045577 | H4C11432.17 | 1921 | 1922 |
| 19 | 6 | 27032749 | 27050124 | H6C3386.1 | 1923 | 1924 |
| 19 | 6 | 27095691 | 27099155 | H6C3392.1 | 1925 | 1926 |
| 19 | 6 | 27096039 | 27099682 | H6C3392.2 | 1927 | 1928 |
| 19 | 6 | 27167207 | 27202211 | H6C3396.1 | 1929 | 1930 |
| 19 | 6 | 27199620 | 27208551 | H6C3401.1 | 1931 | 1932 |
| 19 | 6 | 27201837 | 27208519 | H6C3401.2 | 1933 | 1934 |
| 19 | 6 | 27201837 | 27208519 | H6C3401.3 | 1935 | 1936 |
| 19 | 6 | 27201837 | 27208520 | H6C3401.4 | 1937 | 1938 |
| 19 | 6 | 27201837 | 27208534 | H6C3401.5 | 1939 | 1940 |
| 19 | 6 | 27203224 | 27208520 | H6C3401.6 | 1941 | 1942 |
| 19 | 6 | 27207746 | 27208554 | H6C3406.1 | 1943 | 1944 |
| 19 | 6 | 27208799 | 27211049 | H6C3407.1 | 1945 | 1946 |
| 19 | 6 | 27213342 | 27222611 | H6C3409.1 | 1947 | 1948 |
| 19 | 6 | 27214051 | 27222611 | H6C3409.2 | 1949 | 1950 |
| 19 | 6 | 27215039 | 27216264 | H6C3411.1 | 1951 | 1952 |
| 19 | 6 | 27217851 | 27221754 | H6C3413.1 | 1953 | 1954 |
| 19 | 6 | 27222863 | 27223325 | H6C3415.1 | 1955 | 1956 |
| 19 | 6 | 27323458 | 27332228 | H6C3422.1 | 1957 | 1958 |
| 19 | 6 | 27323458 | 27332377 | H6C3422.2 | 1959 | 1960 |
| 19 | 6 | 27323486 | 27332228 | H6C3422.3 | 1961 | 1962 |
| 19 | 6 | 27323486 | 27332377 | H6C3422.4 | 1963 | 1964 |
| 19 | 6 | 27326828 | 27332228 | H6C3422.5 | 1965 | 1966 |
| 19 | 6 | 27326828 | 27332377 | H6C3422.6 | 1967 | 1968 |
| 19 | 6 | 27327079 | 27332228 | H6C3422.7 | 1969 | 1970 |
| 19 | 6 | 27327079 | 27332377 | H6C3422.8 | 1971 | 1972 |
| 19 | 6 | 27327389 | 27331298 | H6C3422.9 | 1973 | 1974 |
| 19 | 6 | 27327389 | 27332228 | H6C3422.10 | 1975 | 1976 |
| 19 | 6 | 27327389 | 27332377 | H6C3422.11 | 1977 | 1978 |
| 19 | 6 | 27328640 | 27332228 | H6C3422.12 | 1979 | 1980 |
| 19 | 6 | 27328640 | 27332382 | H6C3422.13 | 1981 | 1982 |
| 19 | 6 | 27361660 | 27387070 | H6C3426.1 | 1983 | 1984 |
| 19 | 6 | 27400518 | 27401721 | H6C3429.1 | 1985 | 1986 |
| 19 | 6 | 27433577 | 27439398 | H6C3430.1 | 1987 | 1988 |
| 19 | 6 | 27433577 | 27447283 | H6C3430.2 | 1989 | 1990 |
| 19 | 6 | 27433581 | 27435982 | H6C3431.1 | 1991 | 1992 |
| 19 | 6 | 27438562 | 27446616 | H6C3430.3 | 1993 | 1994 |
| 19 | 6 | 27450392 | 27477326 | H6C3436.1 | 1995 | 1996 |
| 20 | 6 | 152220799 | 152517103 | H6C15161.1 | 1997 | 1998 |
| 20 | 6 | 152220927 | 152512543 | H6C15161.3 | 1999 | 2000 |
| 20 | 6 | 152220927 | 152512543 | H6C15161.4 | 2001 | 2002 |
| 20 | 6 | 152220927 | 152512543 | H6C15161.5 | 2003 | 2004 |
| 20 | 6 | 152222355 | 152542850 | H6C15161.7 | 2005 | 2006 |
| 20 | 6 | 152534932 | 152545465 | H6C15198.1 | 2007 | 2008 |
| 20 | 6 | 152534932 | 152561012 | H6C15199.1 | 2009 | 2010 |
| 20 | 6 | 152534932 | 152581536 | H6C15199.2 | 2011 | 2012 |
| 20 | 6 | 152534932 | 152592715 | H6C15199.3 | 2013 | 2014 |
| 20 | 6 | 152534932 | 152724416 | H6C15199.4 | 2015 | 2016 |
| 20 | 6 | 152534932 | 152731593 | H6C15199.5 | 2017 | 2018 |
| 20 | 6 | 152534932 | 152731628 | H6C15199.6 | 2019 | 2020 |
| 20 | 6 | 152534932 | 153050100 | H6C15199.7 | 2021 | 2022 |
| 20 | 6 | 152534932 | 153050648 | H6C15199.8 | 2023 | 2024 |
| 20 | 6 | 152535006 | 152581536 | H6C15199.9 | 2025 | 2026 |
| 20 | 6 | 152535006 | 152592715 | H6C15199.10 | 2027 | 2028 |
| 20 | 6 | 152535006 | 152637892 | H6C15199.11 | 2029 | 2030 |
| 20 | 6 | 152535006 | 152724416 | H6C15199.12 | 2031 | 2032 |
| 20 | 6 | 152535006 | 152731593 | H6C15199.13 | 2033 | 2034 |
| 20 | 6 | 152535006 | 153050648 | H6C15199.14 | 2035 | 2036 |
| 20 | 6 | 152691351 | 152695777 | H6C15199.15 | 2037 | 2038 |
| 20 | 6 | 152728470 | 152747437 | H6C15199.16 | 2039 | 2040 |
| 20 | 6 | 152748527 | 152794613 | H6C15199.17 | 2041 | 2042 |
| 20 | 6 | 152780241 | 152794613 | H6C15199.18 | 2043 | 2044 |
| 20 | 6 | 152793778 | 152794959 | H6C15254.1 | 2045 | 2046 |
| 20 | 6 | 152815646 | 152818211 | H6C15256.1 | 2047 | 2048 |
| 20 | 6 | 152959180 | 152960210 | H6C15265.1 | 2049 | 2050 |
| 20 | 6 | 153096548 | 153102408 | H6C15273.1 | 2051 | 2052 |
| 20 | 6 | 153111144 | 153137827 | H6C15274.1 | 2053 | 2054 |
| 20 | 6 | 153111144 | 153137967 | H6C15274.2 | 2055 | 2056 |
| 20 | 6 | 153111149 | 153137827 | H6C15274.3 | 2057 | 2058 |
| 20 | 6 | 153111194 | 153137827 | H6C15274.4 | 2059 | 2060 |
| 20 | 6 | 153111194 | 153137967 | H6C15274.5 | 2061 | 2062 |
| 20 | 6 | 153126913 | 153135896 | H6C15274.6 | 2063 | 2064 |
| 20 | 6 | 153164045 | 153173012 | H6C15280.1 | 2065 | 2066 |
| 20 | 6 | 153207744 | 153245162 | H6C15281.1 | 2067 | 2068 |
| 20 | 6 | 153218001 | 153218372 | H6C15281.2 | 2069 | 2070 |
| 20 | 6 | 153276832 | 153281697 | H6C15285.1 | 2071 | 2072 |
| 21 | 7 | 30327977 | 30339064 | H7C3483.1 | 2073 | 2074 |
| 21 | 7 | 30329167 | 30375681 | H7C3483.2 | 2075 | 2076 |
| 21 | 7 | 30338586 | 30346469 | H7C3490.1 | 2077 | 2078 |
| 21 | 7 | 30362655 | 30364587 | H7C3501.1 | 2079 | 2080 |
| 21 | 7 | 30363175 | 30365076 | H7C3483.3 | 2081 | 2082 |
| 21 | 7 | 30363175 | 30390615 | H7C3483.4 | 2083 | 2084 |
| 21 | 7 | 30370951 | 30385944 | H7C3483.5 | 2085 | 2086 |
| 21 | 7 | 30372848 | 30377751 | H7C3483.6 | 2087 | 2088 |
| 21 | 7 | 30374264 | 30376806 | H7C3507.1 | 2089 | 2090 |
| 21 | 7 | 30379259 | 30398671 | H7C3483.7 | 2091 | 2092 |
| 21 | 7 | 30381343 | 30387531 | H7C3483.8 | 2093 | 2094 |
| 21 | 7 | 30387163 | 30398671 | H7C3483.9 | 2095 | 2096 |
| 21 | 7 | 30387504 | 30390635 | H7C3483.10 | 2097 | 2098 |
| 21 | 7 | 30387559 | 30407664 | H7C3483.11 | 2099 | 2100 |
| 21 | 7 | 30390378 | 30398828 | H7C3483.12 | 2101 | 2102 |
| 21 | 7 | 30407420 | 30446889 | H7C3515.1 | 2103 | 2104 |
| 21 | 7 | 30407703 | 30446889 | H7C3515.2 | 2105 | 2106 |
| 21 | 7 | 30427111 | 30434534 | H7C3515.3 | 2107 | 2108 |
| 21 | 7 | 30443369 | 30446889 | H7C3515.4 | 2109 | 2110 |
| 21 | 7 | 30444307 | 30446889 | H7C3515.5 | 2111 | 2112 |
| 21 | 7 | 30465439 | 30495298 | H7C3529.1 | 2113 | 2114 |
| 21 | 7 | 30466102 | 30512959 | H7C3529.2 | 2115 | 2116 |
| 21 | 7 | 30495075 | 30512901 | H7C3529.3 | 2117 | 2118 |
| 21 | 7 | 30510913 | 30558570 | H7C3543.1 | 2119 | 2120 |
| 21 | 7 | 30564990 | 30570458 | H7C3546.1 | 2121 | 2122 |
| 21 | 7 | 30584272 | 30652169 | H7C3546.2 | 2123 | 2124 |
| 21 | 7 | 30584276 | 30705241 | H7C3546.3 | 2125 | 2126 |
| 21 | 7 | 30592389 | 30599511 | H7C3546.4 | 2127 | 2128 |
| 21 | 7 | 30609546 | 30616266 | H7C3552.1 | 2129 | 2130 |
| 21 | 7 | 30665897 | 30685633 | H7C3546.5 | 2131 | 2132 |
| 21 | 7 | 30669774 | 30705241 | H7C3546.6 | 2133 | 2134 |
| 21 | 7 | 30669774 | 30705647 | H7C3546.7 | 2135 | 2136 |
| 21 | 7 | 30724707 | 30738371 | H7C3564.1 | 2137 | 2138 |
| 21 | 7 | 30724707 | 30738373 | H7C3564.2 | 2139 | 2140 |
| 21 | 7 | 30724707 | 30738373 | H7C3564.3 | 2141 | 2142 |
| 21 | 7 | 30776875 | 30792381 | H7C3568.1 | 2143 | 2144 |
| 21 | 7 | 30782387 | 30792374 | H7C3568.2 | 2145 | 2146 |
| 21 | 7 | 30782387 | 30792374 | H7C3568.3 | 2147 | 2148 |
| 21 | 7 | 30782387 | 30792374 | H7C3568.4 | 2149 | 2150 |
| 21 | 7 | 30782387 | 30792374 | H7C3568.5 | 2151 | 2152 |
| 21 | 7 | 30784823 | 30798071 | H7C3568.6 | 2153 | 2154 |
| 21 | 7 | 30784883 | 30788813 | H7C3568.7 | 2155 | 2156 |
| 21 | 7 | 30865381 | 30920152 | H7C3574.1 | 2157 | 2158 |
| 21 | 7 | 30921868 | 30924333 | H7C3574.2 | 2159 | 2160 |
| 21 | 7 | 30921868 | 30924569 | H7C3574.3 | 2161 | 2162 |
| 22 | 7 | 32308387 | 32852274 | H7C3646.3 | 2163 | 2164 |
| 22 | 7 | 32680443 | 32704646 | H7C3724.2 | 2165 | 2166 |
| 22 | 7 | 32681014 | 32704646 | H7C3724.4 | 2167 | 2168 |
| 22 | 7 | 32681851 | 32704646 | H7C3724.6 | 2169 | 2170 |
| 22 | 7 | 32694934 | 32699868 | H7C3730.1 | 2171 | 2172 |
| 22 | 7 | 32698777 | 32701069 | H7C3733.1 | 2173 | 2174 |
| 22 | 7 | 32725706 | 32726984 | H7C3737.1 | 2175 | 2176 |
| 22 | 7 | 32728468 | 32735044 | H7C3739.1 | 2177 | 2178 |
| 22 | 7 | 32728468 | 32755815 | H7C3739.2 | 2179 | 2180 |
| 22 | 7 | 32728468 | 32755858 | H7C3739.3 | 2181 | 2182 |
| 22 | 7 | 32728468 | 32755910 | H7C3739.4 | 2183 | 2184 |
| 22 | 7 | 32728468 | 32756016 | H7C3739.5 | 2185 | 2186 |
| 22 | 7 | 32728468 | 32756020 | H7C3739.6 | 2187 | 2188 |
| 22 | 7 | 32728468 | 32756028 | H7C3739.7 | 2189 | 2190 |
| 22 | 7 | 32728468 | 32756036 | H7C3739.8 | 2191 | 2192 |
| 22 | 7 | 32728468 | 32756289 | H7C3739.9 | 2193 | 2194 |
| 22 | 7 | 32729662 | 32735044 | H7C3739.10 | 2195 | 2196 |
| 22 | 7 | 32729662 | 32755815 | H7C3739.11 | 2197 | 2198 |
| 22 | 7 | 32729662 | 32755910 | H7C3739.12 | 2199 | 2200 |
| 22 | 7 | 32729662 | 32756016 | H7C3739.13 | 2201 | 2202 |
| 22 | 7 | 32729662 | 32756020 | H7C3739.14 | 2203 | 2204 |
| 22 | 7 | 32729662 | 32756028 | H7C3739.15 | 2205 | 2206 |
| 22 | 7 | 32729662 | 32756036 | H7C3739.16 | 2207 | 2208 |
| 22 | 7 | 32729662 | 32756289 | H7C3739.17 | 2209 | 2210 |
| 22 | 7 | 32729666 | 32755858 | H7C3739.18 | 2211 | 2212 |
| 22 | 7 | 32734189 | 32756036 | H7C3739.19 | 2213 | 2214 |
| 22 | 7 | 32770264 | 32819783 | H7C3747.1 | 2215 | 2216 |
| 22 | 7 | 32791314 | 32792420 | H7C3748.1 | 2217 | 2218 |
| 22 | 7 | 32818036 | 32819779 | H7C3762.1 | 2219 | 2220 |
| 22 | 7 | 32818081 | 32819780 | H7C3763.1 | 2221 | — |
| 22 | 7 | 32826965 | 32830487 | H7C3766.1 | 2222 | 2223 |
| 22 | 7 | 32826965 | 32853758 | H7C3766.2 | 2224 | 2225 |
| 22 | 7 | 32826965 | 32875646 | H7C3766.3 | 2226 | 2227 |
| 22 | 7 | 32826965 | 32875646 | H7C3766.4 | 2228 | 2229 |
| 22 | 7 | 32830290 | 32834064 | H7C3770.1 | 2230 | 2231 |
| 22 | 7 | 32876051 | 32908369 | H7C3788.1 | 2232 | 2233 |
| 22 | 7 | 32907648 | 32926257 | H7C3791.1 | 2234 | 2235 |
| 22 | 7 | 32907650 | 32910458 | H7C3791.2 | 2236 | 2237 |
| 22 | 7 | 32907650 | 32912832 | H7C3791.3 | 2238 | 2239 |
| 22 | 7 | 32907650 | 32913738 | H7C3791.4 | 2240 | 2241 |
| 22 | 7 | 32911922 | 32922150 | H7C3791.5 | 2242 | 2243 |
| 22 | 7 | 32942144 | 32965602 | H7C3796.1 | 2244 | 2245 |
| 22 | 7 | 32942144 | 33347222 | H7C3796.2 | 2246 | 2247 |
| 22 | 7 | 32942144 | 33418482 | H7C3796.3 | 2248 | 2249 |
| 22 | 7 | 32942144 | 33418920 | H7C3796.4 | 2250 | 2251 |
| 22 | 7 | 32942144 | 33418920 | H7C3796.5 | 2252 | 2253 |
| 22 | 7 | 33163254 | 33418482 | H7C3796.6 | 2254 | 2255 |
| 22 | 7 | 33163254 | 33418920 | H7C3796.7 | 2256 | 2257 |
| 22 | 7 | 33318460 | 33418482 | H7C3796.8 | 2258 | 2259 |
| 22 | 7 | 33318460 | 33418920 | H7C3796.9 | 2260 | 2261 |
| 22 | 7 | 33346538 | 33418482 | H7C3796.10 | 2262 | 2263 |
| 22 | 7 | 33346538 | 33418920 | H7C3796.11 | 2264 | 2265 |
| 22 | 7 | 33367750 | 33418482 | H7C3796.12 | 2266 | 2267 |
| 22 | 7 | 33367750 | 33418920 | H7C3796.13 | 2268 | 2269 |
| 22 | 7 | 33477459 | 33481782 | H7C3834.1 | 2270 | 2271 |
| 22 | 7 | 33538832 | 33541308 | H7C3842.1 | 2272 | 2273 |
| 22 | 7 | 33606019 | 33615982 | H7C3853.1 | 2274 | 2275 |
| 22 | 7 | 33607914 | 33616008 | H7C3853.2 | 2276 | 2277 |
| 23 | 7 | 126886152 | 127326611 | H7C13659.1 | 2278 | 2279 |
| 23 | 7 | 126886152 | 127326777 | H7C13659.2 | 2280 | 2281 |
| 23 | 7 | 126932896 | 127326611 | H7C13668.1 | 2282 | 2283 |
| 23 | 7 | 126932896 | 127326777 | H7C13668.2 | 2284 | 2285 |
| 23 | 7 | 127163186 | 127326609 | H7C13715.1 | 2286 | 2287 |
| 23 | 7 | 127163186 | 127326777 | H7C13715.2 | 2288 | 2289 |
| 23 | 7 | 127260857 | 127264201 | H7C13745.1 | 2290 | 2291 |
| 23 | 7 | 127260857 | 127264953 | H7C13746.1 | 2292 | 2293 |
| 23 | 7 | 127261076 | 127264953 | H7C13746.2 | 2294 | 2295 |
| 23 | 7 | 127262507 | 127264953 | H7C13746.3 | 2296 | 2297 |
| 23 | 7 | 127320473 | 127326611 | H7C13659.6 | 2298 | 2299 |
| 23 | 7 | 127321014 | 127326611 | H7C13668.3 | 2300 | 2301 |
| 23 | 7 | 127322881 | 127326611 | H7C13659.7 | 2302 | 2303 |
| 23 | 7 | 127322881 | 127326777 | H7C13659.8 | 2304 | 2305 |
| 23 | 7 | 127325406 | 127326611 | H7C13659.9 | 2306 | 2307 |
| 23 | 7 | 127325406 | 127326777 | H7C13659.10 | 2308 | 2309 |
| 23 | 7 | 127485992 | 127491633 | H7C13787.1 | 2310 | 2311 |
| 23 | 7 | 127538118 | 127541883 | H7C13795.1 | 2312 | 2313 |
| 23 | 7 | 127538177 | 127541883 | H7C13795.2 | 2314 | 2315 |
| 23 | 7 | 127544372 | 127577913 | H7C13798.1 | 2316 | 2317 |
| 23 | 7 | 127544529 | 127577913 | H7C13798.2 | 2318 | 2319 |
| 23 | 7 | 127584329 | 127595603 | H7C13808.1 | 2320 | 2321 |
| 23 | 7 | 127584329 | 127595686 | H7C13808.2 | 2322 | 2323 |
| 23 | 7 | 127584329 | 127595690 | H7C13808.3 | 2324 | 2325 |
| 23 | 7 | 127584329 | 127595690 | H7C13808.4 | 2326 | 2327 |
| 23 | 7 | 127620651 | 127626350 | H7C13810.1 | 2328 | 2329 |
| 23 | 7 | 127620651 | 127626877 | H7C13810.2 | 2330 | 2331 |
| 23 | 7 | 127620686 | 127626350 | H7C13810.3 | 2332 | 2333 |
| 23 | 7 | 127620686 | 127626877 | H7C13810.4 | 2334 | 2335 |
| 23 | 7 | 127626281 | 127639947 | H7C13812.1 | 2336 | 2337 |
| 23 | 7 | 127626281 | 127643491 | H7C13812.2 | 2338 | 2339 |
| 23 | 7 | 127626281 | 127643491 | H7C13812.3 | 2340 | 2341 |
| 23 | 7 | 127626281 | 127643988 | H7C13812.4 | 2342 | 2343 |
| 23 | 7 | 127626281 | 127643992 | H7C13812.5 | 2344 | 2345 |
| 23 | 7 | 127626281 | 127644257 | H7C13812.6 | 2346 | 2347 |
| 23 | 7 | 127667352 | 127668156 | H7C13821.1 | 2348 | 2349 |
| 23 | 7 | 127689834 | 127691914 | H7C13826.1 | 2350 | 2351 |
| 23 | 7 | 127689834 | 127692422 | H7C13826.2 | 2352 | 2353 |
| 23 | 7 | 127689834 | 127693132 | H7C13826.3 | 2354 | 2355 |
| 23 | 7 | 127689853 | 127691914 | H7C13826.4 | 2356 | 2357 |
| 23 | 7 | 127689853 | 127692422 | H7C13826.5 | 2358 | 2359 |
| 23 | 7 | 127689853 | 127693132 | H7C13826.6 | 2360 | 2361 |
| 23 | 7 | 127689895 | 127692422 | H7C13827.1 | 2362 | 2363 |
| 23 | 7 | 127689895 | 127693132 | H7C13827.2 | 2364 | 2365 |
| 23 | 7 | 127689907 | 127692422 | H7C13828.1 | 2366 | 2367 |
| 23 | 7 | 127689907 | 127693132 | H7C13828.2 | 2368 | 2369 |
| 23 | 7 | 127689941 | 127703132 | H7C13826.7 | 2370 | 2371 |
| 23 | 7 | 127691193 | 127693132 | H7C13830.1 | 2372 | 2373 |
| 23 | 7 | 127703719 | 127704481 | H7C13834.1 | 2374 | 2375 |
| 23 | 7 | 127704707 | 127707474 | H7C13835.1 | 2376 | 2377 |
| 23 | 7 | 127710752 | 127722307 | H7C13838.1 | 2378 | 2379 |
| 23 | 7 | 127710752 | 127736928 | H7C13838.2 | 2380 | 2381 |
| 23 | 7 | 127710758 | 127738459 | H7C13838.3 | 2382 | 2383 |
| 23 | 7 | 127713178 | 127739320 | H7C13839.1 | 2384 | 2385 |
| 24 | 7 | 145251098 | 147552823 | H7C16453.1 | 2386 | 2387 |
| 24 | 7 | 145251100 | 147555735 | H7C16453.2 | 2388 | 2389 |
| 24 | 7 | 146215675 | 146232349 | H7C16520.1 | 2390 | 2391 |
| 24 | 7 | 146774013 | 147112922 | H7C16453.3 | 2392 | 2393 |
| 24 | 7 | 146806418 | 146807884 | H7C16536.1 | 2394 | 2395 |
| 24 | 7 | 146806450 | 146807883 | H7C16536.2 | 2396 | 2397 |
| 24 | 7 | 147179335 | 147307218 | H7C16453.4 | 2398 | 2399 |
| 24 | 7 | 147474201 | 147552823 | H7C16453.5 | 2400 | 2401 |
| 24 | 7 | 147549489 | 147552823 | H7C16453.6 | 2402 | 2403 |
| 24 | 7 | 147553109 | 147555738 | H7C16558.1 | 2404 | 2405 |
| 24 | 7 | 147569286 | 147580921 | H7C16560.1 | 2406 | 2407 |
| 24 | 7 | 147575739 | 147632097 | H7C16562.1 | 2408 | 2409 |
| 24 | 7 | 147678271 | 147679225 | H7C16566.1 | 2410 | — |
| 24 | 7 | 147715140 | 147715539 | H7C16568.1 | 2411 | 2412 |
| 24 | 7 | 147722422 | 147723840 | H7C16569.1 | 2413 | 2414 |
| 24 | 7 | 147725304 | 147750599 | H7C16571.1 | 2415 | 2416 |
| 24 | 7 | 147771939 | 147772332 | H7C16573.1 | 2417 | 2418 |
| 25 | 7 | 149378653 | 149458310 | H7C16887.1 | 2419 | 2420 |
| 25 | 7 | 149444277 | 149472893 | H7C16905.1 | 2421 | 2422 |
| 25 | 7 | 149457946 | 149463973 | H7C16905.2 | 2423 | 2424 |
| 25 | 7 | 149457947 | 149472893 | H7C16905.3 | 2425 | 2426 |
| 25 | 7 | 149458035 | 149472893 | H7C16905.4 | 2427 | 2428 |
| 25 | 7 | 149458275 | 149472893 | H7C16905.5 | 2429 | 2430 |
| 25 | 7 | 149464523 | 149467459 | H7C16912.1 | 2431 | 2432 |
| 25 | 7 | 149466114 | 149466825 | H7C16913.1 | 2433 | 2434 |
| 25 | 7 | 149471462 | 149472892 | H7C16916.1 | 2435 | 2436 |
| 25 | 7 | 149471462 | 149472892 | H7C16917.1 | 2437 | 2438 |
| 25 | 7 | 149471462 | 149472893 | H7C16918.1 | 2439 | 2440 |
| 25 | 7 | 149471466 | 149472893 | H7C16919.1 | 2441 | 2442 |
| 25 | 7 | 149471529 | 149472892 | H7C16920.1 | 2443 | 2444 |
| 25 | 7 | 149471920 | 149472893 | H7C16916.2 | 2445 | 2446 |
| 25 | 7 | 149471920 | 149472893 | H7C16921.1 | 2447 | 2448 |
| 25 | 7 | 149473054 | 149476384 | H7C16922.1 | 2449 | 2450 |
| 25 | 7 | 149473054 | 149476397 | H7C16922.2 | 2451 | 2452 |
| 25 | 7 | 149473054 | 149476421 | H7C16922.3 | 2453 | 2454 |
| 25 | 7 | 149473054 | 149476425 | H7C16922.4 | 2455 | 2456 |
| 25 | 7 | 149473059 | 149473848 | H7C16922.5 | 2457 | 2458 |
| 25 | 7 | 149503532 | 149508782 | H7C16930.1 | 2459 | 2460 |
| 25 | 7 | 149503542 | 149508782 | H7C16930.2 | 2461 | 2462 |
| 25 | 7 | 149503543 | 149508782 | H7C16930.3 | 2463 | 2464 |
| 25 | 7 | 149503546 | 149508782 | H7C16930.4 | 2465 | 2466 |
| 25 | 7 | 149503553 | 149508782 | H7C16930.5 | 2467 | 2468 |
| 25 | 7 | 149503554 | 149508782 | H7C16930.6 | 2469 | 2470 |
| 25 | 7 | 149503562 | 149508782 | H7C16930.7 | 2471 | 2472 |
| 25 | 7 | 149503569 | 149508782 | H7C16930.8 | 2473 | 2474 |
| 25 | 7 | 149503590 | 149508782 | H7C16930.9 | 2475 | 2476 |
| 25 | 7 | 149503597 | 149508782 | H7C16930.10 | 2477 | 2478 |
| 25 | 7 | 149503614 | 149508782 | H7C16930.11 | 2479 | 2480 |
| 25 | 7 | 149503628 | 149508782 | H7C16930.12 | 2481 | 2482 |
| 25 | 7 | 149503831 | 149508782 | H7C16930.13 | 2483 | 2484 |
| 25 | 7 | 149504447 | 149508782 | H7C16932.1 | 2485 | 2486 |
| 25 | 7 | 149504857 | 149508782 | H7C16934.1 | 2487 | 2488 |
| 25 | 7 | 149505080 | 149508782 | H7C16935.1 | 2489 | 2490 |
| 25 | 7 | 149505257 | 149508782 | H7C16936.1 | 2491 | 2492 |
| 25 | 7 | 149505771 | 149508782 | H7C16937.1 | 2493 | 2494 |
| 25 | 7 | 149505905 | 149508779 | H7C16938.1 | 2495 | 2496 |
| 25 | 7 | 149506907 | 149508218 | H7C16939.1 | 2497 | 2498 |
| 25 | 7 | 149507354 | 149508780 | H7C16940.1 | 2499 | 2500 |
| 25 | 7 | 149507737 | 149508780 | H7C16941.1 | 2501 | 2502 |
| 25 | 7 | 149514053 | 149533369 | H7C16943.1 | 2503 | 2504 |
| 25 | 7 | 149514097 | 149533369 | H7C16943.2 | 2505 | 2506 |
| 25 | 7 | 149514581 | 149518621 | H7C16944.1 | 2507 | 2508 |
| 25 | 7 | 149531245 | 149533367 | H7C16948.1 | 2509 | 2510 |
| 25 | 7 | 149535437 | 149545696 | H7C16950.1 | 2511 | 2512 |
| 25 | 7 | 149535437 | 149547206 | H7C16950.2 | 2513 | 2514 |
| 25 | 7 | 149539881 | 149542656 | H7C16950.3 | 2515 | 2516 |
| 25 | 7 | 149540176 | 149545696 | H7C16950.4 | 2517 | 2518 |
| 25 | 7 | 149540176 | 149547206 | H7C16950.5 | 2519 | 2520 |
| 25 | 7 | 149540472 | 149542963 | H7C16950.6 | 2521 | 2522 |
| 25 | 7 | 149541339 | 149547205 | H7C16954.1 | 2523 | 2524 |
| 25 | 7 | 149542108 | 149542656 | H7C16950.7 | 2525 | 2526 |
| 25 | 7 | 149542157 | 149545696 | H7C16950.8 | 2527 | 2528 |
| 25 | 7 | 149542157 | 149547206 | H7C16950.9 | 2529 | 2530 |
| 25 | 7 | 149542732 | 149545696 | H7C16950.10 | 2531 | 2532 |
| 25 | 7 | 149542732 | 149547206 | H7C16950.11 | 2533 | 2534 |
| 25 | 7 | 149542772 | 149545696 | H7C16950.12 | 2535 | 2536 |
| 25 | 7 | 149542772 | 149547206 | H7C16950.13 | 2537 | 2538 |
| 25 | 7 | 149568389 | 149582525 | H7C16960.1 | 2539 | 2540 |
| 25 | 7 | 149568389 | 149582876 | H7C16960.2 | 2541 | 2542 |
| 25 | 7 | 149585376 | 149614130 | H7C16962.1 | 2543 | 2544 |
| 25 | 7 | 149647294 | 149648498 | H7C16970.1 | 2545 | 2546 |
| 25 | 7 | 149649565 | 149655808 | H7C16971.1 | 2547 | 2548 |
| 25 | 7 | 149702099 | 149708691 | H7C16974.1 | 2549 | 2550 |
| 25 | 7 | 149702105 | 149708691 | H7C16974.2 | 2551 | 2552 |
| 25 | 7 | 149702143 | 149708691 | H7C16974.3 | 2553 | 2554 |
| 25 | 7 | 149760110 | 149767030 | H7C16976.1 | 2555 | 2556 |
| 25 | 7 | 149760110 | 149767044 | H7C16976.2 | 2557 | 2558 |
| 25 | 7 | 149760110 | 149767055 | H7C16976.3 | 2559 | 2560 |
| 25 | 7 | 149760110 | 149767091 | H7C16976.4 | 2561 | 2562 |
| 25 | 7 | 149820435 | 149828267 | H7C16979.1 | 2563 | 2564 |
| 25 | 7 | 149820435 | 149828380 | H7C16979.2 | 2565 | 2566 |
| 25 | 7 | 149820459 | 149825259 | H7C16979.3 | 2567 | 2568 |
| 25 | 7 | 149820462 | 149824438 | H7C16979.4 | 2569 | 2570 |
| 25 | 7 | 149820476 | 149855899 | H7C16979.5 | 2571 | 2572 |
| 25 | 7 | 149820476 | 149856480 | H7C16979.6 | 2573 | 2574 |
| 25 | 7 | 149851337 | 149855899 | H7C16979.7 | 2575 | 2576 |
| 25 | 7 | 149851337 | 149856529 | H7C16979.8 | 2577 | 2578 |
| 25 | 7 | 149851346 | 149855899 | H7C16979.9 | 2579 | 2580 |
| 25 | 7 | 149851346 | 149856480 | H7C16979.10 | 2581 | 2582 |
| 25 | 7 | 149851368 | 149877831 | H7C16979.11 | 2583 | 2584 |
| 25 | 7 | 149851368 | 149878317 | H7C16979.12 | 2585 | 2586 |
| 25 | 7 | 149851368 | 149878385 | H7C16979.13 | 2587 | 2588 |
| 25 | 7 | 149851368 | 149878799 | H7C16979.14 | 2589 | 2590 |
| 25 | 7 | 149872083 | 149877807 | H7C16979.15 | 2591 | 2592 |
| 25 | 7 | 149872083 | 149878317 | H7C16979.16 | 2593 | 2594 |
| 25 | 7 | 149872083 | 149878385 | H7C16979.17 | 2595 | 2596 |
| 25 | 7 | 149872083 | 149878799 | H7C16979.18 | 2597 | 2598 |
| 25 | 7 | 149872170 | 149878385 | H7C16979.19 | 2599 | 2600 |
| 25 | 7 | 149877063 | 149878799 | H7C16988.1 | 2601 | 2602 |
| 25 | 7 | 149877085 | 149878317 | H7C16989.1 | 2603 | 2604 |
| 25 | 7 | 149882118 | 149884651 | H7C16990.1 | 2605 | 2606 |
| 25 | 7 | 149884471 | 149925485 | H7C16991.1 | 2607 | 2608 |
| 25 | 7 | 149895884 | 149913823 | H7C16993.1 | 2609 | 2610 |
| 25 | 7 | 149900460 | 149916797 | H7C16993.2 | 2611 | 2612 |
| 25 | 7 | 149911943 | 149915849 | H7C16993.3 | 2613 | 2614 |
| 25 | 7 | 149925936 | 149928002 | H7C16999.1 | 2615 | 2616 |
| 25 | 7 | 149926022 | 149933345 | H7C16999.2 | 2617 | 2618 |
| 25 | 7 | 149926022 | 149935105 | H7C16999.3 | 2619 | 2620 |
| 25 | 7 | 149926022 | 149935269 | H7C16999.4 | 2621 | 2622 |
| 25 | 7 | 149926022 | 149936098 | H7C16999.5 | 2623 | 2624 |
| 25 | 7 | 149926022 | 149936971 | H7C16999.6 | 2625 | 2626 |
| 25 | 7 | 149926025 | 149928002 | H7C16999.7 | 2627 | 2628 |
| 25 | 7 | 149926027 | 149926333 | H7C17001.1 | 2629 | 2630 |
| 25 | 7 | 149926551 | 149935269 | H7C16999.8 | 2631 | 2632 |
| 25 | 7 | 149928689 | 149935983 | H7C16999.9 | 2633 | 2634 |
| 25 | 7 | 149928692 | 149934424 | H7C16999.10 | 2635 | 2636 |
| 25 | 7 | 149928692 | 149935408 | H7C16999.11 | 2637 | 2638 |
| 25 | 7 | 149935488 | 149939856 | H7C17005.1 | 2639 | 2640 |
| 25 | 7 | 149935506 | 149939855 | H7C17005.2 | 2641 | 2642 |
| 25 | 7 | 149935523 | 149937062 | H7C17005.3 | 2643 | 2644 |
| 25 | 7 | 149935525 | 149939856 | H7C17005.4 | 2645 | 2646 |
| 25 | 7 | 149935533 | 149936898 | H7C17005.5 | 2647 | 2648 |
| 25 | 7 | 149936264 | 149939856 | H7C17005.6 | 2649 | 2650 |
| 25 | 7 | 149936264 | 149939856 | H7C17005.7 | 2651 | 2652 |
| 25 | 7 | 149936791 | 149939854 | H7C17005.8 | 2653 | 2654 |
| 25 | 7 | 149936872 | 149939856 | H7C17005.9 | 2655 | 2656 |
| 25 | 7 | 149937995 | 149939856 | H7C17005.10 | 2657 | 2658 |
| 25 | 7 | 149938059 | 149939854 | H7C17005.11 | 2659 | 2660 |
| 25 | 7 | 149939196 | 149939854 | H7C17005.12 | 2661 | 2662 |
| 26 | 7 | 150079691 | 150090565 | H7C17017.1 | 2663 | 2664 |
| 26 | 7 | 150079691 | 150111060 | H7C17017.2 | 2665 | 2666 |
| 26 | 7 | 150079691 | 150113051 | H7C17017.3 | 2667 | 2668 |
| 26 | 7 | 150079696 | 150098803 | H7C17018.1 | 2669 | 2670 |
| 26 | 7 | 150084145 | 150090565 | H7C17017.4 | 2671 | 2672 |
| 26 | 7 | 150084145 | 150109582 | H7C17017.5 | 2673 | 2674 |
| 26 | 7 | 150084145 | 150113051 | H7C17017.6 | 2675 | 2676 |
| 26 | 7 | 150084169 | 150109582 | H7C17017.7 | 2677 | 2678 |
| 26 | 7 | 150125752 | 150149334 | H7C17023.1 | 2679 | 2680 |
| 26 | 7 | 150136694 | 150138670 | H7C17023.2 | 2681 | 2682 |
| 26 | 7 | 150145268 | 150149334 | H7C17023.3 | 2683 | 2684 |
| 26 | 7 | 150145268 | 150149363 | H7C17023.4 | 2685 | 2686 |
| 26 | 7 | 150146971 | 150147858 | H7C17031.1 | 2687 | 2688 |
| 26 | 7 | 150148589 | 150150362 | H7C17033.1 | 2689 | 2690 |
| 26 | 7 | 150149362 | 150159234 | H7C17033.2 | 2691 | 2692 |
| 26 | 7 | 150149606 | 150150732 | H7C17033.3 | 2693 | 2694 |
| 26 | 7 | 150155652 | 150158244 | H7C17033.4 | 2695 | 2696 |
| 26 | 7 | 150163157 | 150187488 | H7C17036.1 | 2697 | 2698 |
| 26 | 7 | 150163176 | 150180302 | H7C17036.2 | 2699 | 2700 |
| 26 | 7 | 150163177 | 150169163 | H7C17036.3 | 2701 | 2702 |
| 26 | 7 | 150163184 | 150180302 | H7C17036.4 | 2703 | 2704 |
| 26 | 7 | 150163184 | 150180302 | H7C17036.5 | 2705 | 2706 |
| 26 | 7 | 150163197 | 150169065 | H7C17036.6 | 2707 | 2708 |
| 26 | 7 | 150169236 | 150172323 | H7C17036.7 | 2709 | 2710 |
| 26 | 7 | 150172422 | 150180302 | H7C17036.8 | 2711 | 2712 |
| 26 | 7 | 150178806 | 150180302 | H7C17036.9 | 2713 | 2714 |
| 26 | 7 | 150183252 | 150187488 | H7C17036.10 | 2715 | 2716 |
| 26 | 7 | 150183252 | 150187488 | H7C17036.11 | 2717 | 2718 |
| 26 | 7 | 150183252 | 150187491 | H7C17036.12 | 2719 | 2720 |
| 26 | 7 | 150183252 | 150187491 | H7C17036.13 | 2721 | 2722 |
| 26 | 7 | 150185654 | 150187489 | H7C17036.14 | 2723 | 2724 |
| 26 | 7 | 150185947 | 150187491 | H7C17036.15 | 2725 | 2726 |
| 26 | 7 | 150185968 | 150187464 | H7C17036.16 | 2727 | 2728 |
| 26 | 7 | 150188546 | 150192648 | H7C17048.1 | 2729 | 2730 |
| 26 | 7 | 150194337 | 150211262 | H7C17050.1 | 2731 | 2732 |
| 26 | 7 | 150194401 | 150211262 | H7C17050.2 | 2733 | 2734 |
| 26 | 7 | 150195985 | 150211262 | H7C17050.3 | 2735 | 2736 |
| 26 | 7 | 150197375 | 150211262 | H7C17050.4 | 2737 | 2738 |
| 26 | 7 | 150203464 | 150204337 | H7C17053.1 | 2739 | 2740 |
| 26 | 7 | 150208341 | 150209859 | H7C17055.1 | 2741 | 2742 |
| 26 | 7 | 150209020 | 150211262 | H7C17050.5 | 2743 | 2744 |
| 26 | 7 | 150211327 | 150215599 | H7C17059.1 | 2745 | 2746 |
| 26 | 7 | 150211354 | 150212701 | H7C17059.2 | 2747 | 2748 |
| 26 | 7 | 150211354 | 150213844 | H7C17059.3 | 2749 | 2750 |
| 26 | 7 | 150211354 | 150215569 | H7C17059.4 | 2751 | 2752 |
| 26 | 7 | 150211354 | 150215591 | H7C17059.5 | 2753 | 2754 |
| 26 | 7 | 150211354 | 150215591 | H7C17059.6 | 2755 | 2756 |
| 26 | 7 | 150211354 | 150215599 | H7C17059.7 | 2757 | 2758 |
| 26 | 7 | 150211354 | 150215599 | H7C17059.8 | 2759 | 2760 |
| 26 | 7 | 150215814 | 150217713 | H7C17063.1 | 2761 | 2762 |
| 26 | 7 | 150215814 | 150217799 | H7C17063.2 | 2763 | 2764 |
| 26 | 7 | 150215814 | 150217941 | H7C17063.3 | 2765 | 2766 |
| 26 | 7 | 150215814 | 150218196 | H7C17063.4 | 2767 | 2768 |
| 26 | 7 | 150215814 | 150218268 | H7C17063.5 | 2769 | 2770 |
| 26 | 7 | 150215820 | 150217710 | H7C17064.1 | 2771 | 2772 |
| 26 | 7 | 150217009 | 150218208 | H7C17063.6 | 2773 | 2774 |
| 26 | 7 | 150220606 | 150279172 | H7C17065.1 | 2775 | 2776 |
| 26 | 7 | 150221473 | 150279172 | H7C17065.2 | 2777 | 2778 |
| 26 | 7 | 150221636 | 150252824 | H7C17065.3 | 2779 | 2780 |
| 26 | 7 | 150249412 | 150258373 | H7C17065.4 | 2781 | 2782 |
| 26 | 7 | 150252842 | 150279172 | H7C17079.1 | 2783 | 2784 |
| 26 | 7 | 150253207 | 150258373 | H7C17065.5 | 2785 | 2786 |
| 26 | 7 | 150253207 | 150258601 | H7C17065.6 | 2787 | 2788 |
| 26 | 7 | 150263330 | 150266045 | H7C17065.7 | 2789 | 2790 |
| 26 | 7 | 150274302 | 150279172 | H7C17065.8 | 2791 | 2792 |
| 26 | 7 | 150278740 | 150279172 | H7C17096.1 | 2793 | 2794 |
| 26 | 7 | 150283323 | 150309221 | H7C17102.1 | 2795 | 2796 |
| 26 | 7 | 150283323 | 150310080 | H7C17102.2 | 2797 | 2798 |
| 26 | 7 | 150310432 | 150321878 | H7C17108.1 | 2799 | 2800 |
| 26 | 7 | 150310432 | 150322126 | H7C17108.2 | 2801 | 2802 |
| 26 | 7 | 150310432 | 150322567 | H7C17108.3 | 2803 | 2804 |
| 26 | 7 | 150325610 | 150327128 | H7C17111.1 | 2805 | 2806 |
| 26 | 7 | 150325610 | 150330304 | H7C17111.2 | 2807 | 2808 |
| 26 | 7 | 150330320 | 150340230 | H7C17112.1 | 2809 | 2810 |
| 26 | 7 | 150342570 | 150345754 | H7C17113.1 | 2811 | 2812 |
| 26 | 7 | 150342570 | 150362314 | H7C17113.2 | 2813 | 2814 |
| 26 | 7 | 150346217 | 150362314 | H7C17113.3 | 2815 | 2816 |
| 26 | 7 | 150348117 | 150362314 | H7C17113.4 | 2817 | 2818 |
| 26 | 7 | 150348235 | 150362314 | H7C17113.5 | 2819 | 2820 |
| 26 | 7 | 150367199 | 150373561 | H7C17119.1 | 2821 | 2822 |
| 26 | 7 | 150367217 | 150373561 | H7C17119.2 | 2823 | 2824 |
| 26 | 7 | 150368130 | 150373553 | H7C17119.3 | 2825 | 2826 |
| 26 | 7 | 150368130 | 150373554 | H7C17119.4 | 2827 | 2828 |
| 26 | 7 | 150371522 | 150373561 | H7C17122.1 | 2829 | 2830 |
| 26 | 7 | 150373099 | 150376159 | H7C17123.1 | 2831 | 2832 |
| 26 | 7 | 150373099 | 150409988 | H7C17123.2 | 2833 | 2834 |
| 26 | 7 | 150373099 | 150410667 | H7C17123.3 | 2835 | 2836 |
| 26 | 7 | 150373099 | 150412417 | H7C17123.4 | 2837 | 2838 |
| 26 | 7 | 150373707 | 150376159 | H7C17123.5 | 2839 | 2840 |
| 26 | 7 | 150373707 | 150383707 | H7C17123.6 | 2841 | 2842 |
| 26 | 7 | 150373707 | 150409988 | H7C17123.7 | 2843 | 2844 |
| 26 | 7 | 150373707 | 150410667 | H7C17123.8 | 2845 | 2846 |
| 26 | 7 | 150373707 | 150412417 | H7C17123.9 | 2847 | 2848 |
| 26 | 7 | 150373708 | 150374387 | H7C17124.1 | 2849 | 2850 |
| 26 | 7 | 150384058 | 150389124 | H7C17128.1 | 2851 | 2852 |
| 26 | 7 | 150476505 | 150512780 | H7C17137.1 | 2853 | 2854 |
| 26 | 7 | 150476505 | 150513193 | H7C17137.2 | 2855 | 2856 |
| 26 | 7 | 150476505 | 150513193 | H7C17137.3 | 2857 | 2858 |
| 26 | 7 | 150486157 | 150498187 | H7C17137.4 | 2859 | 2860 |
| 26 | 7 | 150497443 | 150500589 | H7C17141.1 | 2861 | 2862 |
| 26 | 7 | 150511793 | 150513307 | H7C17148.1 | 2863 | 2864 |
| 26 | 7 | 150511793 | 150513446 | H7C17149.1 | 2865 | 2866 |
| 26 | 7 | 150512569 | 150515152 | H7C17150.1 | 2867 | 2868 |
| 26 | 7 | 150515854 | 150545130 | H7C17151.1 | 2869 | 2870 |
| 26 | 7 | 150515854 | 150545388 | H7C17151.2 | 2871 | 2872 |
| 26 | 7 | 150541138 | 150543613 | H7C17151.3 | 2873 | 2874 |
| 26 | 7 | 150541138 | 150543613 | H7C17163.1 | 2875 | 2876 |
| 26 | 7 | 150541138 | 150545130 | H7C17151.4 | 2877 | 2878 |
| 26 | 7 | 150543293 | 150545435 | H7C17151.5 | 2879 | 2880 |
| 26 | 7 | 150543894 | 150548451 | H7C17166.1 | 2881 | 2882 |
| 26 | 7 | 150543969 | 150549822 | H7C17166.2 | 2883 | 2884 |
| 26 | 7 | 150544018 | 150547778 | H7C17166.3 | 2885 | 2886 |
| 26 | 7 | 150544776 | 150546741 | H7C17166.4 | 2887 | 2888 |
| 27 | 7 | 156629195 | 156709609 | H7C17932.1 | 2889 | 2890 |
| 27 | 7 | 156650752 | 156709609 | H7C17931.1 | 2891 | 2892 |
| 27 | 7 | 156670109 | 156678494 | H7C17931.2 | 2893 | 2894 |
| 27 | 7 | 156715403 | 156716871 | H7C17962.1 | 2895 | 2896 |
| 27 | 7 | 156733487 | 156762953 | H7C17963.1 | 2897 | 2898 |
| 27 | 7 | 156758398 | 156791886 | H7C17964.1 | 2899 | 2900 |
| 27 | 7 | 156793491 | 156795758 | H7C17966.1 | 2901 | 2902 |
| 27 | 7 | 156805463 | 156809011 | H7C17967.1 | 2903 | 2904 |
| 27 | 7 | 156808735 | 156810286 | H7C17969.1 | 2905 | 2906 |
| 27 | 7 | 156817952 | 156819495 | H7C17971.1 | 2907 | 2908 |
| 27 | 7 | 156831222 | 156832923 | H7C17972.1 | 2909 | 2910 |
| 27 | 7 | 156831222 | 156861202 | H7C17973.1 | 2911 | 2912 |
| 27 | 7 | 156831222 | 156861380 | H7C17973.2 | 2913 | 2914 |
| 27 | 7 | 156831222 | 157833944 | H7C17973.3 | 2915 | 2916 |
| 27 | 7 | 156831222 | 157879894 | H7C17973.4 | 2917 | 2918 |
| 27 | 7 | 156831222 | 157879894 | H7C17973.5 | 2919 | 2920 |
| 27 | 7 | 156831222 | 157879941 | H7C17973.6 | 2921 | 2922 |
| 27 | 7 | 156831226 | 156832720 | H7C17974.1 | 2923 | 2924 |
| 27 | 7 | 156832683 | 157879894 | H7C17973.7 | 2925 | 2926 |
| 27 | 7 | 156887274 | 156896226 | H7C17973.8 | 2927 | 2928 |
| 27 | 7 | 156894679 | 156906074 | H7C17973.9 | 2929 | 2930 |
| 27 | 7 | 156906190 | 156910933 | H7C17991.1 | 2931 | 2932 |
| 27 | 7 | 157023976 | 157024625 | H7C18011.1 | 2933 | 2934 |
| 27 | 7 | 157146696 | 157149976 | H7C18029.1 | 2935 | 2936 |
| 27 | 7 | 157146752 | 157158256 | H7C18029.2 | 2937 | 2938 |
| 27 | 7 | 157150208 | 157151566 | H7C18030.1 | 2939 | 2940 |
| 27 | 7 | 157319540 | 157323296 | H7C18058.1 | 2941 | 2942 |
| 27 | 7 | 157392592 | 157403414 | H7C17973.10 | 2943 | 2944 |
| 27 | 7 | 157480076 | 157480760 | H7C18070.1 | 2945 | 2946 |
| 27 | 7 | 157609075 | 157879938 | H7C17973.11 | 2947 | 2948 |
| 27 | 7 | 157697780 | 157698578 | H7C18094.1 | 2949 | 2950 |
| 27 | 7 | 157740773 | 157879929 | H7C17973.12 | 2951 | 2952 |
| 27 | 7 | 157912777 | 157917108 | H7C18126.1 | 2953 | 2954 |
| 27 | 7 | 157923197 | 157939349 | H7C18128.1 | 2955 | 2956 |
| 27 | 7 | 157923197 | 157967756 | H7C18128.2 | 2957 | 2958 |
| 27 | 7 | 157923197 | 157996933 | H7C18128.3 | 2959 | 2960 |
| 27 | 7 | 157923477 | 157939349 | H7C18128.4 | 2961 | 2962 |
| 27 | 7 | 157923477 | 157967756 | H7C18128.5 | 2963 | 2964 |
| 27 | 7 | 157923477 | 157995871 | H7C18128.6 | 2965 | 2966 |
| 27 | 7 | 157923477 | 157996933 | H7C18128.7 | 2967 | 2968 |
| 27 | 7 | 157923477 | 157996977 | H7C18128.8 | 2969 | 2970 |
| 27 | 7 | 157931963 | 157996977 | H7C18128.9 | 2971 | 2972 |
| 27 | 7 | 157985579 | 157988818 | H7C18128.10 | 2973 | 2974 |
| 27 | 7 | 158011914 | 158012337 | H7C18134.1 | 2975 | 2976 |
| 27 | 7 | 158023161 | 158025088 | H7C18137.1 | 2977 | 2978 |
| 27 | 7 | 158023161 | 158035230 | H7C18138.1 | 2979 | 2980 |
| 27 | 7 | 158023161 | 158037211 | H7C18138.2 | 2981 | 2982 |
| 27 | 7 | 158023161 | 158039825 | H7C18136.1 | 2983 | 2984 |
| 27 | 7 | 158023161 | 158110917 | H7C18138.3 | 2985 | 2986 |
| 27 | 7 | 158023161 | 158121664 | H7C18138.4 | 2987 | 2988 |
| 27 | 7 | 158023161 | 158121795 | H7C18138.5 | 2989 | 2990 |
| 27 | 7 | 158025610 | 158035230 | H7C18138.6 | 2991 | 2992 |
| 27 | 7 | 158025610 | 158037211 | H7C18138.7 | 2993 | 2994 |
| 27 | 7 | 158025610 | 158110917 | H7C18138.8 | 2995 | 2996 |
| 27 | 7 | 158025768 | 158037211 | H7C18138.9 | 2997 | 2998 |
| 27 | 7 | 158025768 | 158110917 | H7C18138.10 | 2999 | 3000 |
| 27 | 7 | 158074659 | 158075421 | H7C18159.1 | 3001 | 3002 |
| 27 | 7 | 158079928 | 158080580 | H7C18138.11 | 3003 | 3004 |
| 28 | 9 | 26791730 | 26795860 | H9C2222.1 | 3005 | 3006 |
| 28 | 9 | 26830575 | 26882801 | H9C2224.1 | 3007 | 3008 |
| 28 | 9 | 26830575 | 26882801 | H9C2224.2 | 3009 | 3010 |
| 28 | 9 | 26830684 | 26882801 | H9C2224.3 | 3011 | 3012 |
| 28 | 9 | 26830684 | 26882801 | H9C2224.4 | 3013 | 3014 |
| 28 | 9 | 26831257 | 26882801 | H9C2224.5 | 3015 | 3016 |
| 28 | 9 | 26831257 | 26882801 | H9C2224.6 | 3017 | 3018 |
| 28 | 9 | 26831257 | 26883155 | H9C2224.7 | 3019 | 3020 |
| 28 | 9 | 26893368 | 26937463 | H9C2240.1 | 3021 | 3022 |
| 28 | 9 | 26895340 | 26937463 | H9C2240.2 | 3023 | 3024 |
| 28 | 9 | 26897562 | 26937463 | H9C2240.3 | 3025 | 3026 |
| 28 | 9 | 26945777 | 26946305 | H9C2254.1 | 3027 | 3028 |
| 28 | 9 | 26946401 | 27045756 | H9C2255.1 | 3029 | 3030 |
| 28 | 9 | 26946401 | 27052930 | H9C2255.2 | 3031 | 3032 |
| 28 | 9 | 26983587 | 26995691 | H9C2265.1 | 3033 | 3034 |
| 28 | 9 | 26985538 | 26987767 | H9C2265.2 | 3035 | 3036 |
| 28 | 9 | 26985541 | 26995691 | H9C2265.3 | 3037 | 3038 |
| 28 | 9 | 27050298 | 27052930 | H9C2255.3 | 3039 | 3040 |
| 28 | 9 | 27092627 | 27094722 | H9C2277.1 | 3041 | 3042 |
| 28 | 9 | 27099235 | 27220171 | H9C2279.1 | 3043 | 3044 |
| 28 | 9 | 27099235 | 27220175 | H9C2279.2 | 3045 | 3046 |
| 28 | 9 | 27235681 | 27272791 | H9C2288.1 | 3047 | 3048 |
| 28 | 9 | 27274655 | 27287137 | H9C2294.1 | 3049 | 3050 |
| 28 | 9 | 27282064 | 27287134 | H9C2294.2 | 3051 | 3052 |
| 28 | 9 | 27282140 | 27287134 | H9C2294.3 | 3053 | 3054 |
| 28 | 9 | 27282395 | 27286998 | H9C2294.4 | 3055 | 3056 |
| 29 | 9 | 70103442 | 70192428 | H9C5133.1 | 3057 | 3058 |
| 29 | 9 | 70103442 | 70196892 | H9C5133.2 | 3059 | 3060 |
| 29 | 9 | 70103442 | 70199357 | H9C5133.3 | 3061 | 3062 |
| 29 | 9 | 70166944 | 70199343 | H9C5148.1 | 3063 | 3064 |
| 29 | 9 | 70178484 | 70185326 | H9C5151.1 | 3065 | 3066 |
| 29 | 9 | 70179578 | 70199357 | H9C5152.1 | 3067 | 3068 |
| 29 | 9 | 70194395 | 70202217 | H9C5155.1 | 3069 | 3070 |
| 29 | 9 | 70229036 | 70259094 | H9C5162.1 | 3071 | 3072 |
| 29 | 9 | 70229068 | 70259094 | H9C5162.2 | 3073 | 3074 |
| 29 | 9 | 70379521 | 70713554 | H9C5181.1 | 3075 | 3076 |
| 29 | 9 | 70379521 | 70713554 | H9C5181.2 | 3077 | 3078 |
| 29 | 9 | 70379521 | 70713554 | H9C5181.3 | 3079 | 3080 |
| 29 | 9 | 70379521 | 70713554 | H9C5181.4 | 3081 | 3082 |
| 29 | 9 | 70379521 | 70713554 | H9C5181.5 | 3083 | 3084 |
| 29 | 9 | 70379521 | 70713554 | H9C5181.6 | 3085 | 3086 |
| 29 | 9 | 70379521 | 70966068 | H9C5181.7 | 3087 | 3088 |
| 29 | 9 | 70435395 | 70447950 | H9C5181.8 | 3089 | 3090 |
| 29 | 9 | 70446309 | 70480062 | H9C5195.1 | 3091 | 3092 |
| 29 | 9 | 70552516 | 70557343 | H9C5208.1 | 3093 | 3094 |
| 29 | 9 | 70570509 | 70575336 | H9C5210.1 | 3095 | 3096 |
| 29 | 9 | 70628330 | 70713554 | H9C5181.9 | 3097 | 3098 |
| 29 | 9 | 70636116 | 70713554 | H9C5181.10 | 3099 | 3100 |
| 29 | 9 | 70668915 | 71291374 | H9C5181.11 | 3101 | 3102 |
| 30 | 9 | 93160509 | 93188243 | H9C7236.1 | 3103 | 3104 |
| 30 | 9 | 93272178 | 93273136 | H9C7246.1 | 3105 | 3106 |
| 30 | 9 | 93277114 | 93278959 | H9C7248.1 | 3107 | 3108 |
| 30 | 9 | 93277894 | 93280662 | H9C7249.1 | 3109 | 3110 |
| 30 | 9 | 93278435 | 93280662 | H9C7249.2 | 3111 | 3112 |
| 30 | 9 | 93280537 | 93293448 | H9C7252.1 | 3113 | 3114 |
| 30 | 9 | 93286681 | 93292386 | H9C7252.2 | 3115 | 3116 |
| 30 | 9 | 93286681 | 93292820 | H9C7252.3 | 3117 | 3118 |
| 30 | 9 | 93286681 | 93293388 | H9C7252.4 | 3119 | 3120 |
| 30 | 9 | 93286681 | 93293448 | H9C7252.5 | 3121 | 3122 |
| 30 | 9 | 93286681 | 93293448 | H9C7252.6 | 3123 | 3124 |
| 30 | 9 | 93286681 | 93293448 | H9C7252.7 | 3125 | 3126 |
| 30 | 9 | 93286681 | 93293448 | H9C7252.8 | 3127 | 3128 |
| 30 | 9 | 93286681 | 93295429 | H9C7252.9 | 3129 | 3130 |
| 30 | 9 | 93288333 | 93292386 | H9C7252.10 | 3131 | 3132 |
| 30 | 9 | 93288333 | 93292820 | H9C7252.11 | 3133 | 3134 |
| 30 | 9 | 93288333 | 93293388 | H9C7252.12 | 3135 | 3136 |
| 30 | 9 | 93288333 | 93293448 | H9C7252.13 | 3137 | 3138 |
| 30 | 9 | 93288333 | 93293448 | H9C7252.14 | 3139 | 3140 |
| 30 | 9 | 93288333 | 93293448 | H9C7252.15 | 3141 | 3142 |
| 30 | 9 | 93288333 | 93293448 | H9C7252.16 | 3143 | 3144 |
| 30 | 9 | 93288333 | 93295429 | H9C7252.17 | 3145 | 3146 |
| 30 | 9 | 93292317 | 93293448 | H9C7252.18 | 3147 | 3148 |
| 30 | 9 | 93292352 | 93293448 | H9C7252.19 | 3149 | 3150 |
| 30 | 9 | 93292361 | 93292804 | H9C7252.20 | 3151 | 3152 |
| 30 | 9 | 93293494 | 93373243 | H9C7257.1 | 3153 | 3154 |
| 30 | 9 | 93293494 | 93407953 | H9C7257.2 | 3155 | 3156 |
| 30 | 9 | 93293494 | 93408238 | H9C7257.3 | 3157 | 3158 |
| 30 | 9 | 93313060 | 93313409 | H9C7262.1 | 3159 | 3160 |
| 30 | 9 | 93380769 | 93402750 | H9C7279.1 | 3161 | 3162 |
| 30 | 9 | 93418463 | 93521424 | H9C7288.1 | 3163 | 3164 |
| 30 | 9 | 93418583 | 93521424 | H9C7288.2 | 3165 | 3166 |
| 30 | 9 | 93418586 | 93521424 | H9C7288.3 | 3167 | 3168 |
| 30 | 9 | 93512980 | 93514127 | H9C7308.1 | 3169 | 3170 |
| 30 | 9 | 93513309 | 93514127 | H9C7308.2 | 3171 | 3172 |
| 30 | 9 | 93515641 | 93518527 | H9C7288.4 | 3173 | 3174 |
| 30 | 9 | 93517412 | 93521336 | H9C7288.5 | 3175 | 3176 |
| 30 | 9 | 93517412 | 93521424 | H9C7288.6 | 3177 | 3178 |
| 30 | 9 | 93547999 | 93574191 | H9C7314.1 | 3179 | 3180 |
| 30 | 9 | 93698860 | 93700170 | H9C7315.1 | 3181 | 3182 |
| 31 | 9 | 110268483 | 110292576 | H9C9036.1 | 3183 | 3184 |
| 31 | 9 | 110273772 | 110276284 | H9C9039.1 | 3185 | 3186 |
| 31 | 9 | 110276191 | 110285457 | H9C9040.1 | 3187 | 3188 |
| 31 | 9 | 110313114 | 110314056 | H9C9043.1 | 3189 | 3190 |
| 31 | 9 | 110317716 | 110355946 | H9C9043.2 | 3191 | 3192 |
| 31 | 9 | 110317716 | 110421715 | H9C9043.3 | 3193 | 3194 |
| 31 | 9 | 110421067 | 110423214 | H9C9065.1 | 3195 | 3196 |
| 31 | 9 | 110441307 | 110447046 | H9C9066.1 | 3197 | 3198 |
| 31 | 9 | 110510605 | 110642833 | H9C9069.1 | 3199 | 3200 |
| 31 | 9 | 110617621 | 110629936 | H9C9069.2 | 3201 | 3202 |
| 31 | 9 | 110715088 | 110879891 | H9C9083.1 | 3203 | 3204 |
| 31 | 9 | 110715088 | 110879920 | H9C9083.2 | 3205 | 3206 |
| 31 | 9 | 110715610 | 110841301 | H9C9083.3 | 3207 | 3208 |
| 31 | 9 | 110715610 | 110879920 | H9C9083.4 | 3209 | 3210 |
| 31 | 9 | 110715610 | 110879920 | H9C9083.5 | 3211 | 3212 |
| 32 | 10 | 19432531 | 19936917 | H10C2456.1 | 3213 | 3214 |
| 32 | 10 | 19963498 | 20063413 | H10C2475.1 | 3215 | 3216 |
| 32 | 10 | 20021244 | 20119336 | H10C2475.2 | 3217 | 3218 |
| 32 | 10 | 20021413 | 20063413 | H10C2475.3 | 3219 | 3220 |
| 32 | 10 | 20039267 | 20057485 | H10C2479.1 | 3221 | 3222 |
| 32 | 10 | 20050522 | 20057457 | H10C2479.2 | 3223 | 3224 |
| 32 | 10 | 20050522 | 20057485 | H10C2479.3 | 3225 | 3226 |
| 32 | 10 | 20062708 | 20064004 | H10C2480.1 | 3227 | 3228 |
| 32 | 10 | 20145352 | 20609127 | H10C2485.1 | 3229 | 3230 |
| 32 | 10 | 20145352 | 20609127 | H10C2485.2 | 3231 | 3232 |
| 32 | 10 | 20145352 | 20609288 | H10C2485.3 | 3233 | 3234 |
| 32 | 10 | 20145352 | 20610607 | H10C2485.4 | 3235 | 3236 |
| 32 | 10 | 20399743 | 20420719 | H10C2541.1 | 3237 | 3238 |
| 33 | 10 | 99884417 | 99994644 | H10C11128.5 | 3239 | 3240 |
| 33 | 10 | 99912628 | 99994644 | H10C11128.8 | 3241 | 3242 |
| 33 | 10 | 99958511 | 99994644 | H10C11128.9 | 3243 | 3244 |
| 33 | 10 | 99997432 | 100017997 | H10C11145.1 | 3245 | 3246 |
| 33 | 10 | 100001769 | 100006319 | H10C11148.1 | 3247 | 3248 |
| 33 | 10 | 100127210 | 100133612 | H10C11158.1 | 3249 | 3250 |
| 33 | 10 | 100133311 | 100141817 | H10C11159.1 | 3251 | 3252 |
| 33 | 10 | 100133312 | 100161750 | H10C11159.2 | 3253 | 3254 |
| 33 | 10 | 100133312 | 100165332 | H10C11159.3 | 3255 | 3256 |
| 33 | 10 | 100163696 | 100164805 | H10C11159.4 | 3257 | 3258 |
| 33 | 10 | 100163696 | 100165566 | H10C11164.1 | 3259 | 3260 |
| 33 | 10 | 100164902 | 100167470 | H10C11166.1 | 3261 | 3262 |
| 33 | 10 | 100164902 | 100168185 | H10C11166.2 | 3263 | 3264 |
| 33 | 10 | 100164902 | 100175409 | H10C11166.3 | 3265 | 3266 |
| 33 | 10 | 100164902 | 100196694 | H10C11166.4 | 3267 | 3268 |
| 33 | 10 | 100164902 | 100196694 | H10C11166.5 | 3269 | 3270 |
| 33 | 10 | 100164902 | 100196694 | H10C11166.6 | 3271 | 3272 |
| 33 | 10 | 100165945 | 100168185 | H10C11166.7 | 3273 | 3274 |
| 33 | 10 | 100165945 | 100175409 | H10C11166.8 | 3275 | 3276 |
| 33 | 10 | 100165945 | 100196694 | H10C11166.10 | 3277 | 3278 |
| 33 | 10 | 100165945 | 100196694 | H10C11166.11 | 3279 | 3280 |
| 33 | 10 | 100165945 | 100196694 | H10C11166.9 | 3281 | 3282 |
| 33 | 10 | 100166763 | 100168185 | H10C11166.12 | 3283 | 3284 |
| 33 | 10 | 100166763 | 100196694 | H10C11166.13 | 3285 | 3286 |
| 33 | 10 | 100178462 | 100196694 | H10C11166.14 | 3287 | 3288 |
| 33 | 10 | 100178894 | 100181265 | H10C11166.15 | 3289 | 3290 |
| 33 | 10 | 100178894 | 100196694 | H10C11166.16 | 3291 | 3292 |
| 33 | 10 | 100179005 | 100196694 | H10C11166.17 | 3293 | 3294 |
| 33 | 10 | 100184961 | 100196673 | H10C11166.18 | 3295 | 3296 |
| 33 | 10 | 100191701 | 100196673 | H10C11166.19 | 3297 | 3298 |
| 33 | 10 | 100191701 | 100196674 | H10C11166.20 | 3299 | 3300 |
| 33 | 10 | 100192677 | 100196673 | H10C11166.21 | 3301 | 3302 |
| 33 | 10 | 100194505 | 100196684 | H10C11166.22 | 3303 | 3304 |
| 33 | 10 | 100196103 | 100203461 | H10C11174.1 | 3305 | 3306 |
| 33 | 10 | 100196103 | 100203550 | H10C11174.2 | 3307 | 3308 |
| 33 | 10 | 100196103 | 100203550 | H10C11174.3 | 3309 | 3310 |
| 33 | 10 | 100208335 | 100216989 | H10C11177.1 | 3311 | 3312 |
| 33 | 10 | 100208335 | 100985549 | H10C11177.2 | 3313 | 3314 |
| 33 | 10 | 100208335 | 100985609 | H10C11177.3 | 3315 | 3316 |
| 33 | 10 | 100208335 | 100985609 | H10C11177.4 | 3317 | 3318 |
| 33 | 10 | 100208335 | 100985609 | H10C11177.5 | 3319 | 3320 |
| 33 | 10 | 101080022 | 101144075 | H10C11198.1 | 3321 | 3322 |
| 33 | 10 | 101107293 | 101144075 | H10C11198.2 | 3323 | 3324 |
| 33 | 10 | 101118077 | 101127275 | H10C11198.3 | 3325 | 3326 |
| 33 | 10 | 101146614 | 101153791 | H10C11206.1 | 3327 | 3328 |
| 33 | 10 | 101146614 | 101181214 | H10C11206.2 | 3329 | 3330 |
| 33 | 10 | 101146859 | 101153791 | H10C11206.3 | 3331 | 3332 |
| 33 | 10 | 101147028 | 101153791 | H10C11206.4 | 3333 | 3334 |
| 33 | 10 | 101147028 | 101181214 | H10C11206.5 | 3335 | 3336 |
| 33 | 10 | 101153176 | 101153775 | H10C11206.6 | 3337 | 3338 |
| 33 | 10 | 101170013 | 101180395 | H10C11206.7 | 3339 | 3340 |
| 33 | 10 | 101180937 | 101185527 | H10C11218.1 | 3341 | 3342 |
| 34 | 11 | 11819545 | 11934579 | H11C1959.1 | 3343 | 3344 |
| 34 | 11 | 11819545 | 11934695 | H11C1959.2 | 3345 | 3346 |
| 34 | 11 | 11819545 | 11935072 | H11C1959.3 | 3347 | 3348 |
| 34 | 11 | 11819545 | 11935647 | H11C1959.4 | 3349 | 3350 |
| 34 | 11 | 11819545 | 11937276 | H11C1959.5 | 3351 | 3352 |
| 34 | 11 | 11819545 | 11937449 | H11C1959.6 | 3353 | 3354 |
| 34 | 11 | 11858308 | 11862825 | H11C1959.7 | 3355 | 3356 |
| 34 | 11 | 11916452 | 11921997 | H11C1959.8 | 3357 | 3358 |
| 34 | 11 | 11928595 | 11935072 | H11C1959.9 | 3359 | 3360 |
| 34 | 11 | 11928595 | 11935647 | H11C1959.10 | 3361 | 3362 |
| 34 | 11 | 11928595 | 11937276 | H11C1959.11 | 3363 | 3364 |
| 34 | 11 | 11928595 | 11937449 | H11C1959.12 | 3365 | 3366 |
| 34 | 11 | 11929819 | 11934579 | H11C1959.13 | 3367 | 3368 |
| 34 | 11 | 11929819 | 11934695 | H11C1959.14 | 3369 | 3370 |
| 34 | 11 | 11929819 | 11935072 | H11C1959.15 | 3371 | 3372 |
| 34 | 11 | 11929819 | 11935647 | H11C1959.16 | 3373 | 3374 |
| 34 | 11 | 11929819 | 11937449 | H11C1959.17 | 3375 | 3376 |
| 34 | 11 | 11940610 | 11986753 | H11C1983.1 | 3377 | 3378 |
| 34 | 11 | 11940610 | 11987205 | H11C1983.2 | 3379 | 3380 |
| 34 | 11 | 11940610 | 11987657 | H11C1983.3 | 3381 | 3382 |
| 34 | 11 | 11941224 | 11986753 | H11C1983.4 | 3383 | 3384 |
| 34 | 11 | 11941224 | 11987205 | H11C1983.5 | 3385 | 3386 |
| 34 | 11 | 11941224 | 11987657 | H11C1983.6 | 3387 | 3388 |
| 34 | 11 | 11956980 | 11987848 | H11C1983.7 | 3389 | 3390 |
| 34 | 11 | 11978983 | 11987436 | H11C1983.8 | 3391 | 3392 |
| 34 | 11 | 11978983 | 11987861 | H11C1983.9 | 3393 | 3394 |
| 34 | 11 | 12008997 | 12039908 | H11C1993.1 | 3395 | 3396 |
| 34 | 11 | 12045051 | 12051476 | H11C1994.1 | 3397 | 3398 |
| 34 | 11 | 12065013 | 12067563 | H11C1995.1 | 3399 | 3400 |
| 34 | 11 | 12072250 | 12074344 | H11C1996.1 | 3401 | 3402 |
| 34 | 11 | 12088708 | 12186491 | H11C1996.2 | 3403 | 3404 |
| 34 | 11 | 12088708 | 12241912 | H11C1996.3 | 3405 | 3406 |
| 34 | 11 | 12185847 | 12241912 | H11C1996.4 | 3407 | 3408 |
| 34 | 11 | 12226324 | 12229700 | H11C2036.1 | 3409 | 3410 |
| 34 | 11 | 12230654 | 12241912 | H11C1996.5 | 3411 | 3412 |
| 34 | 11 | 12231356 | 12241912 | H11C1996.6 | 3413 | 3414 |
| 35 | 11 | 19328846 | 20099723 | H11C2915.1 | 3415 | 3416 |
| 35 | 11 | 19480787 | 19485354 | H11C2930.1 | 3417 | 3418 |
| 35 | 11 | 19489012 | 19502201 | H11C2932.1 | 3419 | 3420 |
| 35 | 11 | 19691487 | 20099723 | H11C2915.2 | 3421 | 3422 |
| 35 | 11 | 19691487 | 20099723 | H11C2915.3 | 3423 | 3424 |
| 35 | 11 | 19691718 | 20096692 | H11C2915.4 | 3425 | 3426 |
| 35 | 11 | 19691741 | 20096692 | H11C2915.5 | 3427 | 3428 |
| 35 | 11 | 19764042 | 19765157 | H11C2960.1 | 3429 | 3430 |
| 35 | 11 | 19956793 | 19959459 | H11C2994.1 | 3431 | 3432 |
| 35 | 11 | 19977965 | 19987177 | H11C2996.1 | 3433 | 3434 |
| 35 | 11 | 19986971 | 20002676 | H11C2997.1 | 3435 | 3436 |
| 35 | 11 | 20000677 | 20096692 | H11C2915.6 | 3437 | 3438 |
| 35 | 11 | 20000677 | 20099723 | H11C2915.7 | 3439 | 3440 |
| 35 | 11 | 20021896 | 20027425 | H11C3004.1 | 3441 | 3442 |
| 35 | 11 | 20026872 | 20099723 | H11C2915.8 | 3443 | 3444 |
| 35 | 11 | 20082332 | 20096692 | H11C2915.9 | 3445 | 3446 |
| 35 | 11 | 20082332 | 20099723 | H11C2915.10 | 3447 | 3448 |
| 35 | 11 | 20097202 | 20098754 | H11C3036.1 | 3449 | 3450 |
| 35 | 11 | 20134335 | 20140938 | H11C3041.1 | 3451 | 3452 |
| 36 | 11 | 95141753 | 95152031 | H11C11109.1 | 3453 | 3454 |
| 36 | 11 | 95141753 | 95162603 | H11C11109.2 | 3455 | 3456 |
| 36 | 11 | 95141753 | 95162603 | H11C11109.3 | 3457 | 3458 |
| 36 | 11 | 95141766 | 95142457 | H11C11110.1 | 3459 | 3460 |
| 36 | 11 | 95141766 | 95162603 | H11C11109.4 | 3461 | 3462 |
| 36 | 11 | 95163154 | 95166360 | H11C11120.1 | 3463 | 3464 |
| 36 | 11 | 95163283 | 95195308 | H11C11120.2 | 3465 | 3466 |
| 36 | 11 | 95163283 | 95203885 | H11C11120.3 | 3467 | 3468 |
| 36 | 11 | 95163283 | 95204358 | H11C11120.4 | 3469 | 3470 |
| 36 | 11 | 95163283 | 95204541 | H11C11120.5 | 3471 | 3472 |
| 36 | 11 | 95163283 | 95205040 | H11C11120.6 | 3473 | 3474 |
| 36 | 11 | 95163283 | 95205498 | H11C11120.7 | 3475 | 3476 |
| 36 | 11 | 95163283 | 95205505 | H11C11120.8 | 3477 | 3478 |
| 36 | 11 | 95163283 | 95205505 | H11C11120.9 | 3479 | 3480 |
| 36 | 11 | 95197724 | 95204541 | H11C11120.10 | 3481 | 3482 |
| 36 | 11 | 95197724 | 95205040 | H11C11120.11 | 3483 | 3484 |
| 36 | 11 | 95197724 | 95205505 | H11C11120.12 | 3485 | 3486 |
| 36 | 11 | 95205693 | 95296920 | H11C11135.1 | 3487 | 3488 |
| 36 | 11 | 95205693 | 95296920 | H11C11135.2 | 3489 | 3490 |
| 36 | 11 | 95205693 | 95297107 | H11C11135.3 | 3491 | 3492 |
| 36 | 11 | 95206889 | 95296905 | H11C11135.4 | 3493 | 3494 |
| 36 | 11 | 95206889 | 95296910 | H11C11135.5 | 3495 | 3496 |
| 36 | 11 | 95206889 | 95296912 | H11C11135.6 | 3497 | 3498 |
| 36 | 11 | 95206889 | 95296919 | H11C11135.7 | 3499 | 3500 |
| 36 | 11 | 95207801 | 95296919 | H11C11135.8 | 3501 | 3502 |
| 36 | 11 | 95349404 | 95716125 | H11C11160.1 | 3503 | 3504 |
| 36 | 11 | 95566562 | 95567514 | H11C11231.1 | 3505 | 3506 |
| 36 | 11 | 95574567 | 95575435 | H11C11235.1 | 3507 | 3508 |
| 36 | 11 | 95725578 | 95744546 | H11C11299.1 | 3509 | 3510 |
| 36 | 11 | 95725578 | 95762694 | H11C11299.2 | 3511 | 3512 |
| 36 | 11 | 95725578 | 95762724 | H11C11299.3 | 3513 | 3514 |
| 36 | 11 | 95725578 | 95762724 | H11C11299.4 | 3515 | 3516 |
| 36 | 11 | 95726254 | 95762724 | H11C11299.5 | 3517 | 3518 |
| 36 | 11 | 95726262 | 95744546 | H11C11299.6 | 3519 | 3520 |
| 36 | 11 | 95726262 | 95762694 | H11C11299.7 | 3521 | 3522 |
| 36 | 11 | 95726267 | 95732094 | H11C11299.8 | 3523 | 3524 |
| 36 | 11 | 95762800 | 95880386 | H11C11316.1 | 3525 | 3526 |
| 36 | 11 | 95763461 | 95766375 | H11C11318.1 | 3527 | 3528 |
| 36 | 11 | 95819873 | 95879690 | H11C11321.1 | 3529 | 3530 |
| 36 | 11 | 95881236 | 95887549 | H11C11326.1 | 3531 | 3532 |
| 36 | 11 | 95881268 | 95944906 | H11C11326.2 | 3533 | 3534 |
| 37 | 12 | 55933811 | 56111055 | H12C7337.2 | 3535 | 3536 |
| 37 | 12 | 56106766 | 56108007 | H12C7383.1 | 3537 | 3538 |
| 37 | 12 | 56114765 | 56131455 | H12C7385.1 | 3539 | 3540 |
| 37 | 12 | 56114765 | 56132108 | H12C7385.2 | 3541 | 3542 |
| 37 | 12 | 56134752 | 56138061 | H12C7389.1 | 3543 | 3544 |
| 37 | 12 | 56135352 | 56138061 | H12C7389.2 | 3545 | 3546 |
| 37 | 12 | 56137537 | 56139852 | H12C7390.1 | 3547 | 3548 |
| 37 | 12 | 56139157 | 56140002 | H12C7390.2 | 3549 | 3550 |
| 37 | 12 | 56140200 | 56152312 | H12C7392.1 | 3551 | 3552 |
| 37 | 12 | 56140200 | 56152739 | H12C7392.2 | 3553 | 3554 |
| 37 | 12 | 56140562 | 56144993 | H12C7392.3 | 3555 | 3556 |
| 37 | 12 | 56150365 | 56152739 | H12C7394.1 | 3557 | 3558 |
| 37 | 12 | 56152214 | 56158116 | H12C7395.1 | 3559 | 3560 |
| 37 | 12 | 56152214 | 56158116 | H12C7395.2 | 3561 | 3562 |
| 37 | 12 | 56152214 | 56159682 | H12C7395.3 | 3563 | 3564 |
| 37 | 12 | 56152305 | 56158116 | H12C7395.4 | 3565 | 3566 |
| 37 | 12 | 56152305 | 56158116 | H12C7395.5 | 3567 | 3568 |
| 37 | 12 | 56152305 | 56159900 | H12C7395.6 | 3569 | 3570 |
| 37 | 12 | 56152305 | 56168864 | H12C7395.7 | 3571 | 3572 |
| 37 | 12 | 56152308 | 56159682 | H12C7395.8 | 3573 | 3574 |
| 37 | 12 | 56156207 | 56156387 | H12C7399.1 | 3575 | 3576 |
| 37 | 12 | 56156412 | 56158116 | H12C7395.9 | 3577 | 3578 |
| 37 | 12 | 56156701 | 56157907 | H12C7395.10 | 3579 | 3580 |
| 37 | 12 | 56165977 | 56167009 | H12C7404.1 | 3581 | 3582 |
| 37 | 12 | 56167343 | 56198244 | H12C7405.1 | 3583 | 3584 |
| 37 | 12 | 56168025 | 56170627 | H12C7405.2 | 3585 | 3586 |
| 37 | 12 | 56168025 | 56196703 | H12C7405.3 | 3587 | 3588 |
| 37 | 12 | 56168025 | 56197182 | H12C7405.4 | 3589 | 3590 |
| 37 | 12 | 56168091 | 56170059 | H12C7405.5 | 3591 | 3592 |
| 37 | 12 | 56168120 | 56196703 | H12C7405.6 | 3593 | 3594 |
| 37 | 12 | 56168120 | 56197182 | H12C7405.7 | 3595 | 3596 |
| 37 | 12 | 56176506 | 56198244 | H12C7409.1 | 3597 | 3598 |
| 37 | 12 | 56192811 | 56196703 | H12C7405.8 | 3599 | 3600 |
| 37 | 12 | 56195038 | 56196703 | H12C7405.9 | 3601 | 3602 |
| 37 | 12 | 56195038 | 56197182 | H12C7405.10 | 3603 | 3604 |
| 37 | 12 | 56195267 | 56198244 | H12C7405.11 | 3605 | 3606 |
| 37 | 12 | 56195268 | 56197182 | H12C7405.12 | 3607 | 3608 |
| 37 | 12 | 56195398 | 56196703 | H12C7405.13 | 3609 | 3610 |
| 37 | 12 | 56195398 | 56197182 | H12C7405.14 | 3611 | 3612 |
| 37 | 12 | 56196330 | 56200561 | H12C7412.1 | 3613 | 3614 |
| 37 | 12 | 56196330 | 56200561 | H12C7412.2 | 3615 | 3616 |
| 37 | 12 | 56196330 | 56200561 | H12C7412.3 | 3617 | 3618 |
| 37 | 12 | 56196330 | 56200561 | H12C7412.4 | 3619 | 3620 |
| 37 | 12 | 56196330 | 56200573 | H12C7412.5 | 3621 | 3622 |
| 37 | 12 | 56196639 | 56200561 | H12C7412.6 | 3623 | 3624 |
| 37 | 12 | 56196639 | 56200561 | H12C7412.7 | 3625 | 3626 |
| 37 | 12 | 56196639 | 56200561 | H12C7412.8 | 3627 | 3628 |
| 37 | 12 | 56196639 | 56200561 | H12C7412.9 | 3629 | 3630 |
| 37 | 12 | 56196639 | 56200573 | H12C7412.10 | 3631 | 3632 |
| 37 | 12 | 56202849 | 56203573 | H12C7417.1 | 3633 | 3634 |
| 37 | 12 | 56202885 | 56210198 | H12C7418.1 | 3635 | 3636 |
| 37 | 12 | 56202885 | 56210199 | H12C7418.2 | 3637 | 3638 |
| 37 | 12 | 56207862 | 56210199 | H12C7418.3 | 3639 | 3640 |
| 37 | 12 | 56209583 | 56227203 | H12C7423.1 | 3641 | 3642 |
| 37 | 12 | 56209583 | 56227204 | H12C7423.2 | 3643 | 3644 |
| 37 | 12 | 56209583 | 56227298 | H12C7423.3 | 3645 | 3646 |
| 37 | 12 | 56209588 | 56210198 | H12C7418.4 | 3647 | 3648 |
| 37 | 12 | 56210195 | 56227203 | H12C7423.4 | 3649 | 3650 |
| 37 | 12 | 56210195 | 56227204 | H12C7423.5 | 3651 | 3652 |
| 37 | 12 | 56210195 | 56227298 | H12C7423.6 | 3653 | 3654 |
| 37 | 12 | 56210201 | 56222039 | H12C7422.1 | 3655 | 3656 |
| 37 | 12 | 56210360 | 56212437 | H12C7423.7 | 3657 | 3658 |
| 37 | 12 | 56210360 | 56227203 | H12C7423.8 | 3659 | 3660 |
| 37 | 12 | 56210360 | 56227298 | H12C7423.9 | 3661 | 3662 |
| 37 | 12 | 56212572 | 56213195 | H12C7423.10 | 3663 | 3664 |
| 37 | 12 | 56212686 | 56227256 | H12C7423.11 | 3665 | 3666 |
| 37 | 12 | 56230078 | 56266682 | H12C7432.1 | 3667 | 3668 |
| 37 | 12 | 56271208 | 56283296 | H12C7439.1 | 3669 | 3670 |
| 37 | 12 | 56271208 | 56283477 | H12C7439.2 | 3671 | 3672 |
| 37 | 12 | 56271208 | 56283477 | H12C7439.3 | 3673 | 3674 |
| 37 | 12 | 56284680 | 56289854 | H12C7447.1 | 3675 | 3676 |
| 37 | 12 | 56284814 | 56288769 | H12C7447.2 | 3677 | 3678 |
| 37 | 12 | 56284814 | 56289854 | H12C7447.3 | 3679 | 3680 |
| 37 | 12 | 56290367 | 56306869 | H12C7453.1 | 3681 | 3682 |
| 37 | 12 | 56291476 | 56297295 | H12C7453.2 | 3683 | 3684 |
| 37 | 12 | 56291962 | 56297295 | H12C7453.3 | 3685 | 3686 |
| 37 | 12 | 56293627 | 56298095 | H12C7454.1 | 3687 | 3688 |
| 37 | 12 | 56294043 | 56295638 | H12C7453.4 | 3689 | 3690 |
| 37 | 12 | 56295660 | 56297295 | H12C7456.1 | 3691 | 3692 |
| 37 | 12 | 56298455 | 56301960 | H12C7457.1 | 3693 | 3694 |
| 37 | 12 | 56299613 | 56300468 | H12C7458.1 | 3695 | 3696 |
| 37 | 12 | 56299959 | 56306869 | H12C7453.5 | 3697 | 3698 |
| 37 | 12 | 56300402 | 56306200 | H12C7453.6 | 3699 | 3700 |
| 37 | 12 | 56300402 | 56306869 | H12C7453.7 | 3701 | 3702 |
| 37 | 12 | 56301094 | 56306200 | H12C7453.8 | 3703 | 3704 |
| 37 | 12 | 56303735 | 56306200 | H12C7453.9 | 3705 | 3706 |
| 37 | 12 | 56303736 | 56305345 | H12C7453.10 | 3707 | 3708 |
| 37 | 12 | 56428271 | 56432508 | H12C7487.13 | 3823 | 3824 |
| 37 | 12 | 56428311 | 56429845 | H12C7487.14 | 3825 | 3826 |
| 37 | 12 | 56428311 | 56431680 | H12C7487.15 | 3827 | 3828 |
| 37 | 12 | 56428311 | 56432403 | H12C7487.16 | 3829 | 3830 |
| 37 | 12 | 56428311 | 56432403 | H12C7487.17 | 3831 | 3832 |
| 37 | 12 | 56428311 | 56432444 | H12C7487.18 | 3833 | 3834 |
| 37 | 12 | 56428311 | 56432508 | H12C7487.19 | 3835 | 3836 |
| 37 | 12 | 56429241 | 56432497 | H12C7487.20 | 3837 | 3838 |
| 37 | 12 | 56431761 | 56432165 | H12C7487.21 | 3839 | 3840 |
| 37 | 12 | 56435147 | 56439465 | H12C7491.1 | 3841 | 3842 |
| 37 | 12 | 56435394 | 56440460 | H12C7491.2 | 3843 | 3844 |
| 37 | 12 | 56437727 | 56439465 | H12C7491.3 | 3845 | 3846 |
| 37 | 12 | 56437727 | 56440460 | H12C7491.4 | 3847 | 3848 |
| 37 | 12 | 56442383 | 56447631 | H12C7496.1 | 3849 | 3850 |
| 37 | 12 | 56442383 | 56447631 | H12C7496.2 | 3851 | 3852 |
| 37 | 12 | 56445488 | 56446449 | H12C7496.3 | 3853 | 3854 |
| 37 | 12 | 56448522 | 56452181 | H12C7501.1 | 3855 | 3856 |
| 37 | 12 | 56448522 | 56452522 | H12C7501.2 | 3857 | 3858 |
| 37 | 12 | 56448522 | 56452602 | H12C7501.3 | 3859 | 3860 |
| 37 | 12 | 56448536 | 56452151 | H12C7501.4 | 3861 | 3862 |
| 37 | 12 | 56448632 | 56452151 | H12C7501.5 | 3863 | 3864 |
| 37 | 12 | 56448632 | 56452522 | H12C7501.6 | 3865 | 3866 |
| 37 | 12 | 56448632 | 56452602 | H12C7501.7 | 3867 | 3868 |
| 37 | 12 | 56448683 | 56452151 | H12C7501.8 | 3869 | 3870 |
| 37 | 12 | 56448683 | 56452522 | H12C7501.9 | 3871 | 3872 |
| 37 | 12 | 56448683 | 56452602 | H12C7501.10 | 3873 | 3874 |
| 37 | 12 | 56450733 | 56452522 | H12C7501.11 | 3875 | 3876 |
| 37 | 12 | 56450806 | 56452144 | H12C7501.12 | 3877 | 3878 |
| 37 | 12 | 56451213 | 56453434 | H12C7503.1 | 3879 | 3880 |
| 37 | 12 | 56452305 | 56462591 | H12C7503.2 | 3881 | 3882 |
| 37 | 12 | 56452649 | 56462451 | H12C7503.3 | 3883 | 3884 |
| 37 | 12 | 56452649 | 56462591 | H12C7503.4 | 3885 | 3886 |
| 37 | 12 | 56452649 | 56462591 | H12C7503.5 | 3887 | 3888 |
| 37 | 12 | 56452705 | 56462591 | H12C7503.6 | 3889 | 3890 |
| 37 | 12 | 56452746 | 56456162 | H12C7503.7 | 3891 | 3892 |
| 37 | 12 | 56462800 | 56476786 | H12C7509.1 | 3893 | 3894 |
| 37 | 12 | 56462800 | 56476786 | H12C7509.2 | 3895 | 3896 |
| 37 | 12 | 56462800 | 56478028 | H12C7509.3 | 3897 | 3898 |
| 37 | 12 | 56462800 | 56478028 | H12C7509.4 | 3899 | 3900 |
| 37 | 12 | 56462800 | 56482747 | H12C7509.5 | 3901 | 3902 |
| 37 | 12 | 56462800 | 56488121 | H12C7509.6 | 3903 | 3904 |
| 37 | 12 | 56462847 | 56478028 | H12C7509.7 | 3905 | 3906 |
| 37 | 12 | 56477110 | 56481467 | H12C7511.1 | 3907 | 3908 |
| 37 | 12 | 56477110 | 56496119 | H12C7511.2 | 3909 | 3910 |
| 37 | 12 | 56477428 | 56481467 | H12C7511.3 | 3911 | 3912 |
| 37 | 12 | 56477428 | 56489229 | H12C7511.4 | 3913 | 3914 |
| 37 | 12 | 56477428 | 56496119 | H12C7511.5 | 3915 | 3916 |
| 37 | 12 | 56485905 | 56489229 | H12C7511.6 | 3917 | 3918 |
| 37 | 12 | 56486183 | 56488255 | H12C7511.7 | 3919 | 3920 |
| 37 | 12 | 56499972 | 56503637 | H12C7524.1 | 3921 | 3922 |
| 37 | 12 | 56499972 | 56504026 | H12C7525.1 | 3923 | 3924 |
| 37 | 12 | 56499972 | 56504593 | H12C7525.2 | 3925 | 3926 |
| 37 | 12 | 56499972 | 56526992 | H12C7525.3 | 3927 | 3928 |
| 37 | 12 | 56499972 | 56526992 | H12C7525.4 | 3929 | 3930 |
| 37 | 12 | 56502848 | 56503637 | H12C7524.2 | 3931 | 3932 |
| 37 | 12 | 56502848 | 56504593 | H12C7525.5 | 3933 | 3934 |
| 37 | 12 | 56502848 | 56526992 | H12C7525.6 | 3935 | 3936 |
| 37 | 12 | 56517141 | 56523811 | H12C7538.1 | 3937 | 3938 |
| 37 | 12 | 56523126 | 56523811 | H12C7545.1 | 3939 | 3940 |
| 37 | 12 | 56549885 | 56576532 | H12C7547.1 | 3941 | 3942 |
| 37 | 12 | 56566410 | 56574002 | H12C7547.2 | 3943 | 3944 |
| 37 | 12 | 56574288 | 56576072 | H12C7550.1 | 3945 | 3946 |
| 37 | 12 | 56611498 | 56616237 | H12C7552.1 | 3947 | 3948 |
| 37 | 12 | 56611573 | 56616129 | H12C7552.2 | 3949 | 3950 |
| 37 | 12 | 56611575 | 56616223 | H12C7552.3 | 3951 | 3952 |
| 37 | 12 | 56611618 | 56616213 | H12C7552.4 | 3953 | 3954 |
| 37 | 12 | 56615096 | 56616113 | H12C7552.5 | 3955 | 3956 |
| 37 | 12 | 56615296 | 56616037 | H12C7552.6 | 3957 | 3958 |
| 37 | 12 | 56621553 | 56637190 | H12C7555.1 | 3959 | 3960 |
| 37 | 12 | 56621553 | 56637320 | H12C7555.2 | 3961 | 3962 |
| 37 | 12 | 56621613 | 56637190 | H12C7555.3 | 3963 | 3964 |
| 37 | 12 | 56621613 | 56637320 | H12C7555.4 | 3965 | 3966 |
| 37 | 12 | 56621716 | 56637190 | H12C7555.5 | 3967 | 3968 |
| 37 | 12 | 56621716 | 56637320 | H12C7555.6 | 3969 | 3970 |
| 37 | 12 | 56629794 | 56637190 | H12C7555.7 | 3971 | 3972 |
| 37 | 12 | 56629794 | 56637320 | H12C7555.8 | 3973 | 3974 |
| 38 | 12 | 81254775 | 81375414 | H12C9730.2 | 3975 | 3976 |
| 38 | 12 | 81330941 | 81375484 | H12C9730.6 | 3977 | 3978 |
| 38 | 12 | 81334757 | 81375414 | H12C9730.7 | 3979 | 3980 |
| 38 | 12 | 81334757 | 81375484 | H12C9730.8 | 3981 | 3982 |
| 38 | 12 | 81334978 | 81375414 | H12C9730.9 | 3983 | 3984 |
| 38 | 12 | 81372113 | 81375413 | H12C9730.10 | 3985 | 3986 |
| 38 | 12 | 81377371 | 81393807 | H12C9742.1 | 3987 | 3988 |
| 38 | 12 | 81401458 | 81402432 | H12C9746.1 | 3989 | 3990 |
| 38 | 12 | 81401458 | 81402435 | H12C9745.1 | 3991 | 3992 |
| 38 | 12 | 81583127 | 82031114 | H12C9755.1 | 3993 | 3994 |
| 38 | 12 | 81792819 | 81862218 | H12C9755.2 | 3995 | 3996 |
| 38 | 12 | 81863219 | 81882608 | H12C9755.3 | 3997 | 3998 |
| 38 | 12 | 82028574 | 82031114 | H12C9819.1 | 3999 | 4000 |
| 38 | 12 | 82047577 | 82048437 | H12C9822.1 | 4001 | 4002 |
| 38 | 12 | 82067836 | 82068992 | H12C9823.1 | 4003 | 4004 |
| 39 | 14 | 49578110 | 49625643 | H14C2841.1 | 4005 | 4006 |
| 39 | 14 | 49620118 | 49629111 | H14C2841.2 | 4007 | 4008 |
| 39 | 14 | 49645091 | 49653382 | H14C2849.1 | 4009 | 4010 |
| 39 | 14 | 49645096 | 49649351 | H14C2849.2 | 4011 | 4012 |
| 39 | 14 | 49645096 | 49653382 | H14C2849.3 | 4013 | 4014 |
| 39 | 14 | 49645096 | 49653382 | H14C2849.4 | 4015 | 4016 |
| 39 | 14 | 49645096 | 49653382 | H14C2849.5 | 4017 | 4018 |
| 39 | 14 | 49650643 | 49652325 | H14C2853.1 | 4019 | 4020 |
| 39 | 14 | 49652434 | 49768351 | H14C2855.1 | 4021 | 4022 |
| 39 | 14 | 49653590 | 49768351 | H14C2855.2 | 4023 | 4024 |
| 39 | 14 | 49692359 | 49695972 | H14C2855.3 | 4025 | 4026 |
| 39 | 14 | 49737505 | 49767830 | H14C2855.4 | 4027 | 4028 |
| 39 | 14 | 49740803 | 49767542 | H14C2855.5 | 4029 | 4030 |
| 39 | 14 | 49748429 | 49751811 | H14C2880.1 | 4031 | 4032 |
| 39 | 14 | 49772371 | 49773664 | H14C2885.1 | 4033 | 4034 |
| 39 | 14 | 49774036 | 49848697 | H14C2887.1 | 4035 | 4036 |
| 39 | 14 | 49781645 | 49783545 | H14C2893.1 | 4037 | 4038 |
| 39 | 14 | 49781645 | 49783650 | H14C2893.2 | 4039 | 4040 |
| 39 | 14 | 49781645 | 49824571 | H14C2892.1 | 4041 | 4042 |
| 39 | 14 | 49782931 | 49848697 | H14C2887.2 | 4043 | 4044 |
| 39 | 14 | 49820398 | 49848640 | H14C2887.3 | 4045 | 4046 |
| 39 | 14 | 49843392 | 49844482 | H14C2898.1 | 4047 | 4048 |
| 39 | 14 | 49848791 | 49861174 | H14C2899.1 | 4049 | 4050 |
| 39 | 14 | 49848795 | 49859362 | H14C2899.2 | 4051 | 4052 |
| 39 | 14 | 49848795 | 49862192 | H14C2899.3 | 4053 | 4054 |
| 39 | 14 | 49848795 | 49862204 | H14C2899.4 | 4055 | 4056 |
| 39 | 14 | 49848795 | 49863718 | H14C2899.5 | 4057 | 4058 |
| 39 | 14 | 49848795 | 49863718 | H14C2899.6 | 4059 | 4060 |
| 39 | 14 | 49848796 | 49863718 | H14C2899.7 | 4061 | 4062 |
| 39 | 14 | 49848810 | 49862192 | H14C2899.8 | 4063 | 4064 |
| 39 | 14 | 49848810 | 49863718 | H14C2899.9 | 4065 | 4066 |
| 39 | 14 | 49855701 | 49872026 | H14C2899.10 | 4067 | 4068 |
| 39 | 14 | 49857786 | 49862192 | H14C2899.11 | 4069 | 4070 |
| 39 | 14 | 49857786 | 49863718 | H14C2899.12 | 4071 | 4072 |
| 39 | 14 | 49862703 | 49864424 | H14C2907.1 | 4073 | 4074 |
| 39 | 14 | 49862703 | 49867356 | H14C2907.2 | 4075 | 4076 |
| 39 | 14 | 49862918 | 49867356 | H14C2908.1 | 4077 | 4078 |
| 39 | 14 | 49865972 | 49932367 | H14C2911.1 | 4079 | 4080 |
| 39 | 14 | 49872460 | 49947118 | H14C2911.2 | 4081 | 4082 |
| 39 | 14 | 49933480 | 49934813 | H14C2927.1 | 4083 | 4084 |
| 39 | 14 | 49954448 | 49987064 | H14C2931.1 | 4085 | 4086 |
| 39 | 14 | 49954448 | 50069126 | H14C2931.2 | 4087 | 4088 |
| 39 | 14 | 49954990 | 50069012 | H14C2931.3 | 4089 | 4090 |
| 39 | 14 | 49954990 | 50069126 | H14C2931.4 | 4091 | 4092 |
| 39 | 14 | 49954990 | 50069126 | H14C2931.5 | 4093 | 4094 |
| 39 | 14 | 49955298 | 50069126 | H14C2931.6 | 4095 | 4096 |
| 39 | 14 | 49956233 | 50069126 | H14C2931.7 | 4097 | 4098 |
| 39 | 14 | 49976440 | 49980505 | H14C2931.8 | 4099 | 4100 |
| 39 | 14 | 49985274 | 49993209 | H14C2941.1 | 4101 | 4102 |
| 39 | 14 | 50022396 | 50041596 | H14C2931.9 | 4103 | 4104 |
| 39 | 14 | 50068969 | 50169532 | H14C2964.1 | 4105 | 4106 |
| 39 | 14 | 50069766 | 50168790 | H14C2964.2 | 4107 | 4108 |
| 39 | 14 | 50069766 | 50169532 | H14C2964.3 | 4109 | 4110 |
| 39 | 14 | 50096493 | 50169532 | H14C2964.4 | 4111 | 4112 |
| 39 | 14 | 50170102 | 50181277 | H14C2979.1 | 4113 | 4114 |
| 39 | 14 | 50170102 | 50202343 | H14C2982.1 | 4115 | 4116 |
| 39 | 14 | 50170102 | 50204806 | H14C2979.2 | 4117 | 4118 |
| 39 | 14 | 50170102 | 50204807 | H14C2979.3 | 4119 | 4120 |
| 39 | 14 | 50170102 | 50205321 | H14C2979.4 | 4121 | 4122 |
| 40 | 14 | 80032573 | 80321596 | H14C7194.2 | 4123 | 4124 |
| 40 | 14 | 80366259 | 80477788 | H14C7194.4 | 4125 | 4126 |
| 40 | 14 | 80477587 | 80478127 | H14C7226.1 | 4127 | 4128 |
| 40 | 14 | 80481652 | 80495614 | H14C7194.5 | 4129 | 4130 |
| 40 | 14 | 80491263 | 80680948 | H14C7228.1 | 4131 | 4132 |
| 40 | 14 | 80491527 | 80645039 | H14C7228.2 | 4133 | 4134 |
| 40 | 14 | 80643129 | 80706511 | H14C7246.1 | 4135 | 4136 |
| 40 | 14 | 80711548 | 80716375 | H14C7258.1 | 4137 | 4138 |
| 40 | 14 | 80712245 | 80716375 | H14C7258.2 | 4139 | 4140 |
| 40 | 14 | 80716102 | 80757047 | H14C7259.1 | 4141 | 4142 |
| 40 | 14 | 80716102 | 80757328 | H14C7259.2 | 4143 | 4144 |
| 40 | 14 | 80716282 | 80757049 | H14C7259.3 | 4145 | 4146 |
| 40 | 14 | 80733323 | 80757474 | H14C7259.4 | 4147 | 4148 |
| 40 | 14 | 80757314 | 80758555 | H14C7268.1 | 4149 | 4150 |
| 40 | 14 | 80757534 | 80759476 | H14C7268.2 | 4151 | 4152 |
| 40 | 14 | 80796751 | 80814020 | H14C7274.1 | 4153 | 4154 |
| 40 | 14 | 80796839 | 80814020 | H14C7274.2 | 4155 | 4156 |
| 40 | 14 | 80804144 | 80813032 | H14C7274.3 | 4157 | 4158 |
| 40 | 14 | 80806535 | 80814915 | H14C7274.4 | 4159 | 4160 |
| 40 | 14 | 80806661 | 80934680 | H14C7274.5 | 4161 | 4162 |
| 40 | 14 | 80860885 | 80862407 | H14C7281.1 | 4163 | 4164 |
| 41 | 16 | 46440725 | 46478543 | H16C6398.1 | 4165 | 4166 |
| 41 | 16 | 46449990 | 46499843 | H16C6399.1 | 4167 | 4168 |
| 41 | 16 | 46674384 | 46738182 | H16C6402.1 | 4169 | 4170 |
| 41 | 16 | 46674384 | 46738182 | H16C6402.2 | 4171 | 4172 |
| 41 | 16 | 46674384 | 46738182 | H16C6402.3 | 4173 | 4174 |
| 41 | 16 | 46674747 | 46738062 | H16C6402.4 | 4175 | 4176 |
| 41 | 16 | 46674747 | 46747430 | H16C6402.5 | 4177 | 4178 |
| 41 | 16 | 46702859 | 46703325 | H16C6402.6 | 4179 | 4180 |
| 41 | 16 | 46711733 | 46719912 | H16C6402.7 | 4181 | 4182 |
| 41 | 16 | 46712651 | 46747405 | H16C6402.8 | 4183 | 4184 |
| 41 | 16 | 46756363 | 46758650 | H16C6404.1 | 4185 | 4186 |
| 41 | 16 | 46758320 | 46778802 | H16C6405.1 | 4187 | 4188 |
| 41 | 16 | 46758320 | 46823683 | H16C6405.2 | 4189 | 4190 |
| 41 | 16 | 46758320 | 46826590 | H16C6405.3 | 4191 | 4192 |
| 41 | 16 | 46758320 | 46826590 | H16C6405.4 | 4193 | 4194 |
| 41 | 16 | 46758320 | 46838806 | H16C6405.5 | 4195 | 4196 |
| 41 | 16 | 46800276 | 46836000 | H16C6405.6 | 4197 | 4198 |
| 41 | 16 | 46806643 | 46815072 | H16C6405.7 | 4199 | 4200 |
| 41 | 16 | 46835711 | 46943412 | H16C6411.1 | 4201 | 4202 |
| 41 | 16 | 46835711 | 46945391 | H16C6411.2 | 4203 | 4204 |
| 41 | 16 | 46835713 | 46945391 | H16C6411.3 | 4205 | 4206 |
| 41 | 16 | 46864694 | 46866659 | H16C6418.1 | 4207 | 4208 |
| 41 | 16 | 46868747 | 46887578 | H16C6421.1 | 4209 | 4210 |
| 41 | 16 | 46938342 | 46945391 | H16C6441.1 | 4211 | 4212 |
| 41 | 16 | 46938898 | 46943412 | H16C6411.4 | 4213 | 4214 |
| 41 | 16 | 46938898 | 46945391 | H16C6411.5 | 4215 | 4216 |
| 41 | 16 | 46944394 | 46947870 | H16C6445.1 | 4217 | 4218 |
| 41 | 16 | 46944394 | 46948443 | H16C6445.2 | 4219 | 4220 |
| 41 | 16 | 46945909 | 46953794 | H16C6447.1 | 4221 | 4222 |
| 41 | 16 | 46946630 | 46954414 | H16C6411.6 | 4223 | 4224 |
| 41 | 16 | 46947204 | 46954414 | H16C6411.7 | 4225 | 4226 |
| 41 | 16 | 46951942 | 46957297 | H16C6454.1 | 4227 | 4228 |
| 41 | 16 | 46951942 | 46957299 | H16C6453.1 | 4229 | 4230 |
| 41 | 16 | 46951942 | 46976231 | H16C6453.2 | 4231 | 4232 |
| 41 | 16 | 46951942 | 46976864 | H16C6453.3 | 4233 | 4234 |
| 41 | 16 | 46951949 | 46955294 | H16C6452.1 | 4235 | 4236 |
| 41 | 16 | 46952465 | 46957299 | H16C6453.4 | 4237 | 4238 |
| 41 | 16 | 46952465 | 46976231 | H16C6453.5 | 4239 | 4240 |
| 41 | 16 | 46952465 | 46976864 | H16C6453.6 | 4241 | 4242 |
| 41 | 16 | 46956191 | 46957297 | H16C6453.7 | 4243 | 4244 |
| 42 | 16 | 73590412 | 73699438 | H16C9637.1 | 4245 | 4246 |
| 42 | 16 | 73590412 | 73699438 | H16C9637.2 | 4247 | 4248 |
| 42 | 16 | 73590412 | 73700110 | H16C9637.3 | 4249 | 4250 |
| 42 | 16 | 73590412 | 73702111 | H16C9637.4 | 4251 | 4252 |
| 42 | 16 | 73590412 | 73702393 | H16C9637.5 | 4253 | 4254 |
| 42 | 16 | 73590435 | 73700110 | H16C9637.6 | 4255 | 4256 |
| 42 | 16 | 73591464 | 73699438 | H16C9637.7 | 4257 | 4258 |
| 42 | 16 | 73591640 | 73699438 | H16C9637.8 | 4259 | 4260 |
| 42 | 16 | 73591640 | 73700110 | H16C9637.9 | 4261 | 4262 |
| 42 | 16 | 73591640 | 73702111 | H16C9637.10 | 4263 | 4264 |
| 42 | 16 | 73591640 | 73702389 | H16C9637.11 | 4265 | 4266 |
| 42 | 16 | 73641125 | 73643359 | H16C9650.1 | 4267 | 4268 |
| 42 | 16 | 73643129 | 73644129 | H16C9652.1 | 4269 | 4270 |
| 42 | 16 | 73691828 | 73693291 | H16C9665.1 | 4271 | 4272 |
| 42 | 16 | 73696196 | 73699438 | H16C9637.12 | 4273 | 4274 |
| 42 | 16 | 73696196 | 73700110 | H16C9637.13 | 4275 | 4276 |
| 42 | 16 | 73696196 | 73702111 | H16C9637.14 | 4277 | 4278 |
| 42 | 16 | 73696196 | 73702389 | H16C9637.15 | 4279 | 4280 |
| 42 | 16 | 73699129 | 73702389 | H16C9667.1 | 4281 | 4282 |
| 42 | 16 | 73703258 | 73708167 | H16C9668.1 | 4283 | 4284 |
| 42 | 16 | 73703258 | 73708169 | H16C9668.2 | 4285 | 4286 |
| 42 | 16 | 73703504 | 73708167 | H16C9668.3 | 4287 | 4288 |
| 42 | 16 | 73705484 | 73708152 | H16C9668.4 | 4289 | 4290 |
| 42 | 16 | 73705557 | 73708150 | H16C9668.5 | 4291 | 4292 |
| 42 | 16 | 73705902 | 73706991 | H16C9668.6 | 4293 | 4294 |
| 42 | 16 | 73739908 | 73740407 | H16C9670.1 | 4295 | 4296 |
| 42 | 16 | 73739925 | 73763480 | H16C9671.1 | 4297 | 4298 |
| 42 | 16 | 73739925 | 73763633 | H16C9671.2 | 4299 | 4300 |
| 42 | 16 | 73795492 | 73798584 | H16C9677.1 | 4301 | 4302 |
| 42 | 16 | 73795494 | 73796882 | H16C9677.2 | 4303 | 4304 |
| 42 | 16 | 73795494 | 73796910 | H16C9677.3 | 4305 | 4306 |
| 42 | 16 | 73810403 | 73816326 | H16C9679.1 | 4307 | 4308 |
| 42 | 16 | 73810403 | 73816796 | H16C9679.2 | 4309 | 4310 |
| 42 | 16 | 73814134 | 73816323 | H16C9679.3 | 4311 | 4312 |
| 42 | 16 | 73814134 | 73816796 | H16C9679.4 | 4313 | 4314 |
| 42 | 16 | 73814165 | 73814448 | H16C9679.5 | 4315 | 4316 |
| 42 | 16 | 73814165 | 73815028 | H16C9679.6 | 4317 | 4318 |
| 42 | 16 | 73816215 | 73817488 | H16C9683.1 | 4319 | 4320 |
| 42 | 16 | 73817781 | 73819602 | H16C9684.1 | 4321 | 4322 |
| 42 | 16 | 73817914 | 73819602 | H16C9684.2 | 4323 | 4324 |
| 42 | 16 | 73818187 | 73819596 | H16C9684.3 | 4325 | 4326 |
| 42 | 16 | 73820402 | 73827902 | H16C9685.1 | 4327 | 4328 |
| 42 | 16 | 73820402 | 73830059 | H16C9685.2 | 4329 | 4330 |
| 42 | 16 | 73820402 | 73843005 | H16C9685.3 | 4331 | 4332 |
| 42 | 16 | 73820402 | 73856614 | H16C9685.4 | 4333 | 4334 |
| 42 | 16 | 73820402 | 73857406 | H16C9685.5 | 4335 | 4336 |
| 42 | 16 | 73820402 | 73859449 | H16C9685.6 | 4337 | 4338 |
| 42 | 16 | 73820429 | 73825677 | H16C9687.1 | 4339 | 4340 |
| 42 | 16 | 73820491 | 73843005 | H16C9685.7 | 4341 | 4342 |
| 42 | 16 | 73820491 | 73857406 | H16C9685.8 | 4343 | 4344 |
| 42 | 16 | 73863452 | 73863989 | H16C9700.1 | 4345 | 4346 |
| 42 | 16 | 73884622 | 73896681 | H16C9703.1 | 4347 | 4348 |
| 42 | 16 | 73884622 | 73897056 | H16C9703.2 | 4349 | 4350 |
| 42 | 16 | 73884622 | 73897944 | H16C9703.3 | 4351 | 4352 |
| 42 | 16 | 73884622 | 73954670 | H16C9703.4 | 4353 | 4354 |
| 42 | 16 | 73884622 | 73968127 | H16C9703.5 | 4355 | 4356 |
| 42 | 16 | 73884622 | 74024928 | H16C9703.6 | 4357 | 4358 |
| 42 | 16 | 73885109 | 73896681 | H16C9703.7 | 4359 | 4360 |
| 42 | 16 | 73885109 | 73897056 | H16C9703.8 | 4361 | 4362 |
| 42 | 16 | 73885109 | 73897887 | H16C9703.9 | 4363 | 4364 |
| 42 | 16 | 73885109 | 73954670 | H16C9703.10 | 4365 | 4366 |
| 42 | 16 | 73885109 | 73968127 | H16C9703.11 | 4367 | 4368 |
| 42 | 16 | 73885109 | 74003341 | H16C9703.12 | 4369 | 4370 |
| 42 | 16 | 73885109 | 74024928 | H16C9703.13 | 4371 | 4372 |
| 42 | 16 | 73913288 | 73916447 | H16C9713.1 | 4373 | 4374 |
| 42 | 16 | 73963603 | 74024928 | H16C9703.14 | 4375 | 4376 |
| 42 | 16 | 73972223 | 73986486 | H16C9726.1 | 4377 | 4378 |
| 42 | 16 | 73986080 | 74024928 | H16C9703.15 | 4379 | 4380 |
| 42 | 16 | 74025234 | 74027791 | H16C9738.1 | 4381 | 4382 |
| 42 | 16 | 74034455 | 74038907 | H16C9740.1 | 4383 | 4384 |
| 42 | 16 | 74034455 | 74044098 | H16C9741.1 | 4385 | 4386 |
| 42 | 16 | 74034455 | 74056088 | H16C9741.2 | 4387 | 4388 |
| 42 | 16 | 74034455 | 74056092 | H16C9741.3 | 4389 | 4390 |
| 42 | 16 | 74034455 | 74056172 | H16C9741.4 | 4391 | 4392 |
| 42 | 16 | 74034637 | 74038907 | H16C9740.2 | 4393 | 4394 |
| 42 | 16 | 74034637 | 74044098 | H16C9741.5 | 4395 | 4396 |
| 42 | 16 | 74034637 | 74056088 | H16C9741.6 | 4397 | 4398 |
| 42 | 16 | 74034637 | 74056088 | H16C9741.7 | 4399 | 4400 |
| 42 | 16 | 74034637 | 74056092 | H16C9741.8 | 4401 | 4402 |
| 42 | 16 | 74034637 | 74056136 | H16C9741.9 | 4403 | 4404 |
| 42 | 16 | 74034637 | 74056172 | H16C9741.10 | 4405 | 4406 |
| 42 | 16 | 74038410 | 74044098 | H16C9741.11 | 4407 | 4408 |
| 42 | 16 | 74038410 | 74056092 | H16C9741.12 | 4409 | 4410 |
| 42 | 16 | 74038410 | 74056136 | H16C9741.13 | 4411 | 4412 |
| 42 | 16 | 74038410 | 74056172 | H16C9741.14 | 4413 | 4414 |
| 42 | 16 | 74042221 | 74056087 | H16C9741.15 | 4415 | 4416 |
| 42 | 16 | 74042628 | 74056087 | H16C9741.16 | 4417 | 4418 |
| 42 | 16 | 74049650 | 74051416 | H16C9746.1 | 4419 | 4420 |
| 42 | 16 | 74064523 | 74086806 | H16C9750.1 | 4421 | 4422 |
| 42 | 16 | 74064525 | 74086427 | H16C9751.1 | 4423 | 4424 |
| 42 | 16 | 74069620 | 74086783 | H16C9751.2 | 4425 | 4426 |
| 43 | 16 | 76379978 | 76571505 | H16C9924.1 | 4427 | 4428 |
| 43 | 16 | 76432276 | 76454021 | H16C9930.1 | 4429 | 4430 |
| 43 | 16 | 76569570 | 76571505 | H16C9940.1 | 4431 | 4432 |
| 43 | 16 | 76606080 | 76650852 | H16C9942.1 | 4433 | 4434 |
| 43 | 16 | 76613945 | 76623501 | H16C9943.1 | 4435 | 4436 |
| 43 | 16 | 76620486 | 76623501 | H16C9943.2 | 4437 | 4438 |
| 43 | 16 | 76690512 | 76691599 | H16C9949.1 | 4439 | 4440 |
| 43 | 16 | 76691051 | 76870096 | H16C9950.1 | 4441 | 4442 |
| 43 | 16 | 76691051 | 76870117 | H16C9950.2 | 4443 | 4444 |
| 43 | 16 | 76691051 | 77371845 | H16C9950.3 | 4445 | 4446 |
| 43 | 16 | 76691051 | 77804074 | H16C9950.4 | 4447 | 4448 |
| 43 | 16 | 76691061 | 76872441 | H16C9950.5 | 4449 | 4450 |
| 43 | 16 | 76691183 | 76870100 | H16C9950.6 | 4451 | 4452 |
| 43 | 16 | 76826060 | 76829508 | H16C9969.1 | 4453 | 4454 |
| 43 | 16 | 76828761 | 76829589 | H16C9969.2 | 4455 | 4456 |
| 43 | 16 | 76847579 | 76849089 | H16C9975.1 | 4457 | 4458 |
| 43 | 16 | 77087323 | 77097966 | H16C9998.1 | 4459 | 4460 |
| 43 | 16 | 77125771 | 77127046 | H16C10016.1 | 4461 | 4462 |
| 43 | 16 | 77158417 | 77158973 | H16C10035.1 | 4463 | 4464 |
| 43 | 16 | 77290533 | 77301013 | H16C10076.1 | 4465 | 4466 |
| 43 | 16 | 77416675 | 77548920 | H16C10120.1 | 4467 | 4468 |
| 43 | 16 | 77587653 | 77596122 | H16C10163.1 | 4469 | 4470 |
| 43 | 16 | 77668773 | 77669626 | H16C10178.1 | 4471 | 4472 |
| 43 | 16 | 77681447 | 77693117 | H16C10180.1 | 4473 | 4474 |
| 43 | 16 | 77787762 | 77804074 | H16C9950.7 | 4475 | 4476 |
| 43 | 16 | 77794020 | 77796551 | H16C10193.1 | 4477 | 4478 |
| 43 | 16 | 77802813 | 77804064 | H16C10197.1 | 4479 | 4480 |
| 43 | 16 | 77802813 | 77804074 | H16C10198.1 | 4481 | 4482 |
| 43 | 16 | 77803000 | 77804064 | H16C10199.1 | 4483 | 4484 |
| 43 | 16 | 77804110 | 77820851 | H16C10200.1 | 4485 | 4486 |
| 44 | 18 | 22521582 | 22537594 | H18C2315.11 | 4487 | 4488 |
| 44 | 18 | 22656776 | 22658070 | H18C2360.1 | 4489 | 4490 |
| 44 | 18 | 22686000 | 22687520 | H18C2365.1 | 4491 | 4492 |
| 44 | 18 | 22686000 | 22696135 | H18C2366.1 | 4493 | 4494 |
| 44 | 18 | 22686000 | 22696810 | H18C2366.2 | 4495 | 4496 |
| 44 | 18 | 22686000 | 22696810 | H18C2366.3 | 4497 | 4498 |
| 44 | 18 | 22686000 | 22699747 | H18C2366.4 | 4499 | 4500 |
| 44 | 18 | 22686000 | 22699747 | H18C2366.5 | 4501 | 4502 |
| 44 | 18 | 22687947 | 22696135 | H18C2366.6 | 4503 | 4504 |
| 44 | 18 | 22687947 | 22696810 | H18C2366.7 | 4505 | 4506 |
| 44 | 18 | 22687947 | 22699747 | H18C2366.8 | 4507 | 4508 |
| 44 | 18 | 22687947 | 22699747 | H18C2366.9 | 4509 | 4510 |
| 44 | 18 | 22699266 | 23024648 | H18C2369.1 | 4511 | 4512 |
| 44 | 18 | 22699269 | 22769908 | H18C2369.2 | 4513 | 4514 |
| 44 | 18 | 22749592 | 22976969 | H18C2374.1 | 4515 | 4516 |
| 44 | 18 | 22749592 | 23019305 | H18C2374.2 | 4517 | 4518 |
| 44 | 18 | 22749592 | 23019305 | H18C2374.3 | 4519 | 4520 |
| 44 | 18 | 22749736 | 22976969 | H18C2374.4 | 4521 | 4522 |
| 44 | 18 | 22749736 | 23019305 | H18C2374.5 | 4523 | 4524 |
| 44 | 18 | 22749736 | 23019305 | H18C2374.6 | 4525 | 4526 |
| 44 | 18 | 22750122 | 23019305 | H18C2374.7 | 4527 | 4528 |
| 44 | 18 | 22750122 | 23019305 | H18C2374.8 | 4529 | 4530 |
| 44 | 18 | 23170340 | 23429126 | H18C2403.1 | 4531 | 4532 |
| 44 | 18 | 23170340 | 23429126 | H18C2403.2 | 4533 | 4534 |
| 44 | 18 | 23170701 | 23226829 | H18C2403.3 | 4535 | 4536 |
| 44 | 18 | 23231913 | 23235865 | H18C2406.1 | 4537 | 4538 |
| 45 | 18 | 61568467 | 61699155 | H18C5401.1 | 4539 | 4540 |
| 45 | 18 | 61568867 | 61682340 | H18C5401.2 | 4541 | 4542 |
| 45 | 18 | 61568867 | 61699154 | H18C5401.3 | 4543 | 4544 |
| 45 | 18 | 61735535 | 61740532 | H18C5415.1 | 4545 | 4546 |
| 46 | 22 | 16449458 | 16486067 | H22C214.1 | 4547 | 4548 |
| 46 | 22 | 16449458 | 16486127 | H22C214.2 | 4549 | 4550 |
| 46 | 22 | 16449458 | 16486142 | H22C214.3 | 4551 | 4552 |
| 46 | 22 | 16449478 | 16452050 | H22C214.4 | 4553 | 4554 |
| 46 | 22 | 16449478 | 16486142 | H22C214.5 | 4555 | 4556 |
| 46 | 22 | 16449479 | 16486067 | H22C214.6 | 4557 | 4558 |
| 46 | 22 | 16449479 | 16486127 | H22C214.7 | 4559 | 4560 |
| 46 | 22 | 16449479 | 16486142 | H22C214.8 | 4561 | 4562 |
| 46 | 22 | 16449479 | 16486142 | H22C214.9 | 4563 | 4564 |
| 46 | 22 | 16449785 | 16486067 | H22C214.10 | 4565 | 4566 |
| 46 | 22 | 16449785 | 16486127 | H22C214.11 | 4567 | 4568 |
| 46 | 22 | 16449785 | 16486142 | H22C214.12 | 4569 | 4570 |
| 46 | 22 | 16451878 | 16486127 | H22C214.13 | 4571 | 4572 |
| 46 | 22 | 16458128 | 16486127 | H22C214.14 | 4573 | 4574 |
| 46 | 22 | 16486235 | 16559740 | H22C222.1 | 4575 | 4576 |
| 46 | 22 | 16495909 | 16561098 | H22C222.2 | 4577 | 4578 |
| 46 | 22 | 16495909 | 16561098 | H22C222.3 | 4579 | 4580 |
| 46 | 22 | 16495909 | 16586456 | H22C222.4 | 4581 | 4582 |
| 46 | 22 | 16495909 | 16586456 | H22C222.5 | 4583 | 4584 |
| 46 | 22 | 16495909 | 16586456 | H22C222.6 | 4585 | 4586 |
| 46 | 22 | 16495909 | 16586456 | H22C222.7 | 4587 | 4588 |
| 46 | 22 | 16495909 | 16586546 | H22C222.10 | 4589 | 4590 |
| 46 | 22 | 16495909 | 16586546 | H22C222.11 | 4591 | 4592 |
| 46 | 22 | 16495909 | 16586546 | H22C222.8 | 4593 | 4594 |
| 46 | 22 | 16495909 | 16586546 | H22C222.9 | 4595 | 4596 |
| 46 | 22 | 16495909 | 16587940 | H22C222.12 | 4597 | 4598 |
| 46 | 22 | 16495909 | 16587940 | H22C222.13 | 4599 | 4600 |
| 46 | 22 | 16495909 | 16587940 | H22C222.14 | 4601 | 4602 |
| 46 | 22 | 16495909 | 16587940 | H22C222.15 | 4603 | 4604 |
| 46 | 22 | 16495909 | 16588231 | H22C222.16 | 4605 | 4606 |
| 46 | 22 | 16495909 | 16588231 | H22C222.17 | 4607 | 4608 |
| 46 | 22 | 16495909 | 16588231 | H22C222.18 | 4609 | 4610 |
| 46 | 22 | 16495909 | 16588231 | H22C222.19 | 4611 | 4612 |
| 46 | 22 | 16496095 | 16586456 | H22C222.20 | 4613 | 4614 |
| 46 | 22 | 16496095 | 16586546 | H22C222.21 | 4615 | 4616 |
| 46 | 22 | 16496095 | 16587940 | H22C222.22 | 4617 | 4618 |
| 46 | 22 | 16496095 | 16588231 | H22C222.23 | 4619 | 4620 |
| 46 | 22 | 16564029 | 16586456 | H22C222.24 | 4621 | 4622 |
| 46 | 22 | 16564029 | 16586546 | H22C222.25 | 4623 | 4624 |
| 46 | 22 | 16564029 | 16587940 | H22C222.26 | 4625 | 4626 |
| 46 | 22 | 16564029 | 16588231 | H22C222.27 | 4627 | 4628 |
| 46 | 22 | 16570932 | 16574491 | H22C238.1 | 4629 | 4630 |
| 46 | 22 | 16583995 | 16588231 | H22C242.1 | 4631 | 4632 |
| 46 | 22 | 16591336 | 16592839 | H22C246.1 | 4633 | 4634 |
| 46 | 22 | 16591405 | 16595471 | H22C247.1 | 4635 | 4636 |
| 46 | 22 | 16591405 | 16631303 | H22C247.2 | 4637 | 4638 |
| 46 | 22 | 16591405 | 16631787 | H22C247.3 | 4639 | 4640 |
| 46 | 22 | 16591405 | 16631806 | H22C247.4 | 4641 | 4642 |
| 46 | 22 | 16591405 | 16631815 | H22C247.5 | 4643 | 4644 |
| 46 | 22 | 16591405 | 16631815 | H22C247.6 | 4645 | 4646 |
| 46 | 22 | 16591405 | 16631815 | H22C247.7 | 4647 | 4648 |
| 46 | 22 | 16591458 | 16595471 | H22C247.8 | 4649 | 4650 |
| 46 | 22 | 16591458 | 16631333 | H22C247.9 | 4651 | 4652 |
| 46 | 22 | 16591458 | 16631787 | H22C247.10 | 4653 | 4654 |
| 46 | 22 | 16591458 | 16631806 | H22C247.11 | 4655 | 4656 |
| 46 | 22 | 16591458 | 16631815 | H22C247.12 | 4657 | 4658 |
| 46 | 22 | 16591458 | 16631815 | H22C247.13 | 4659 | 4660 |
| 46 | 22 | 16591458 | 16631815 | H22C247.14 | 4661 | 4662 |
| 46 | 22 | 16591460 | 16607495 | H22C247.15 | 4663 | 4664 |
| 46 | 22 | 16591460 | 16631815 | H22C247.16 | 4665 | 4666 |
| 46 | 22 | 16592531 | 16598514 | H22C247.17 | 4667 | 4668 |
| 46 | 22 | 16592531 | 16631303 | H22C247.18 | 4669 | 4670 |
| 46 | 22 | 16592531 | 16631815 | H22C247.19 | 4671 | 4672 |
| 46 | 22 | 16592531 | 16631815 | H22C247.20 | 4673 | 4674 |
| 46 | 22 | 16592590 | 16631815 | H22C247.21 | 4675 | 4676 |
| 46 | 22 | 16592693 | 16631303 | H22C247.22 | 4677 | 4678 |
| 46 | 22 | 16592693 | 16631787 | H22C247.23 | 4679 | 4680 |
| 46 | 22 | 16592693 | 16631815 | H22C247.24 | 4681 | 4682 |
| 46 | 22 | 16592693 | 16631815 | H22C247.25 | 4683 | 4684 |
| 46 | 22 | 16592696 | 16601297 | H22C247.26 | 4685 | 4686 |
| 46 | 22 | 16592696 | 16631815 | H22C247.27 | 4687 | 4688 |
| 46 | 22 | 16634681 | 16636800 | H22C261.1 | 4689 | 4690 |
| 46 | 22 | 16637180 | 16637883 | H22C262.1 | 4691 | 4692 |
| 46 | 22 | 16644967 | 16669095 | H22C263.1 | 4693 | 4694 |
| 46 | 22 | 16644967 | 16685103 | H22C263.2 | 4695 | 4696 |
| 46 | 22 | 16644968 | 16646266 | H22C264.1 | 4697 | 4698 |
| 46 | 22 | 16644968 | 16647610 | H22C266.1 | 4699 | 4700 |
| 46 | 22 | 16648351 | 16675316 | H22C263.3 | 4701 | 4702 |
| 46 | 22 | 16655993 | 16657308 | H22C272.1 | 4703 | 4704 |
| 46 | 22 | 16656011 | 16657390 | H22C273.1 | 4705 | 4706 |
| 46 | 22 | 16676183 | 16689247 | H22C263.4 | 4707 | 4708 |
| 46 | 22 | 16678762 | 16679688 | H22C263.5 | 4709 | 4710 |
| 46 | 22 | 16691654 | 16764155 | H22C292.1 | 4711 | 4712 |
| 46 | 22 | 16691657 | 16764155 | H22C292.2 | 4713 | 4714 |
| 46 | 22 | 16696299 | 16858821 | H22C292.3 | 4715 | 4716 |
| 46 | 22 | 16709476 | 16720104 | H22C298.1 | 4717 | 4718 |
| 46 | 22 | 16752560 | 16754105 | H22C292.4 | 4719 | 4720 |
| 46 | 22 | 16756401 | 16763982 | H22C292.5 | 4721 | 4722 |
| 46 | 22 | 16861604 | 16864628 | H22C349.1 | 4723 | 4724 |
| 46 | 22 | 16886704 | 16895288 | H22C352.1 | 4725 | 4726 |
| 46 | 22 | 16933846 | 16935007 | H22C354.1 | 4727 | 4728 |
| 46 | 22 | 16933846 | 16935117 | H22C353.1 | 4729 | 4730 |
| 46 | 22 | 16935239 | 16948350 | H22C356.1 | 4731 | 4732 |
| 46 | 22 | 16935242 | 16948790 | H22C356.2 | 4733 | 4734 |
| 46 | 22 | 16935258 | 16945781 | H22C356.3 | 4735 | 4736 |
| 46 | 22 | 16935313 | 16945779 | H22C356.4 | 4737 | 4738 |
| 46 | 22 | 16940952 | 16948350 | H22C357.1 | 4739 | 4740 |
| 46 | 22 | 16940952 | 16948759 | H22C357.2 | 4741 | 4742 |
| 46 | 22 | 16942483 | 16945936 | H22C356.5 | 4743 | 4744 |
| 46 | 22 | 16945214 | 16948350 | H22C359.1 | 4745 | 4746 |
| 46 | 22 | 16945214 | 16948759 | H22C359.2 | 4747 | 4748 |
| 46 | 22 | 16945533 | 16950882 | H22C360.1 | 4749 | 4750 |
| 46 | 22 | 16946289 | 16950882 | H22C361.1 | 4751 | 4752 |
| 46 | 22 | 16946470 | 16948759 | H22C362.1 | 4753 | 4754 |
| 46 | 22 | 16947053 | 16948759 | H22C357.3 | 4755 | 4756 |
| 46 | 22 | 16947053 | 16948759 | H22C364.1 | 4757 | 4758 |
| 46 | 22 | 16947109 | 16948350 | H22C357.4 | 4759 | 4760 |
| 46 | 22 | 16967795 | 16988476 | H22C371.1 | 4761 | 4762 |
| 46 | 22 | 16967795 | 16989052 | H22C371.2 | 4763 | 4764 |
| 46 | 22 | 16978427 | 16988428 | H22C371.3 | 4765 | 4766 |
| 47 | X | 80175344 | 80263530 | HXC4994.1 | 4767 | 4768 |
| 47 | X | 80175344 | 80263557 | HXC4994.2 | 4769 | 4770 |
| 47 | X | 80175344 | 80263586 | HXC4994.3 | 4771 | 4772 |
| 47 | X | 80175647 | 80180122 | HXC4994.4 | 4773 | 4774 |
| 47 | X | 80175709 | 80180122 | HXC4994.5 | 4775 | 4776 |
| 47 | X | 80175709 | 80263586 | HXC4994.6 | 4777 | 4778 |
| 47 | X | 80263587 | 80360191 | HXC4997.1 | 4779 | 4780 |
| 47 | X | 80263621 | 80359238 | HXC4997.2 | 4781 | 4782 |
| 47 | X | 80263621 | 80360191 | HXC4997.3 | 4783 | 4784 |
| 47 | X | 80263661 | 80360191 | HXC4997.4 | 4785 | 4786 |
| 47 | X | 80263715 | 80359238 | HXC4997.5 | 4787 | 4788 |
| 47 | X | 80263862 | 80359238 | HXC4997.6 | 4789 | 4790 |
| 47 | X | 80263862 | 80360191 | HXC4997.7 | 4791 | 4792 |
| 47 | X | 80264244 | 80359238 | HXC4997.8 | 4793 | 4794 |
| 47 | X | 80264244 | 80360191 | HXC4997.9 | 4795 | 4796 |
| 47 | X | 80273145 | 80359238 | HXC4997.10 | 4797 | 4798 |
| 47 | X | 80273145 | 80360191 | HXC4997.11 | 4799 | 4800 |
| 47 | X | 80317137 | 80360191 | HXC4997.12 | 4801 | 4802 |
| 47 | X | 80356945 | 80358119 | HXC5008.1 | 4803 | 4804 |

TABLE 7

| List of significantly associated haplotypes based on the longevity fine mapping results using the Quebec Founder Population (QFP). Individual | | | | | | | | | | | | | | | | | | | |
|---|---|---|---|---|---|---|---|---|---|---|---|---|---|---|---|---|---|---|---|
| haplotypes with associated relative risks are presented in each row of the table; these values were extracted from the associated marker | | | | | | | | | | | | | | | | | | | |
| haplotype window with the most significant p value for each SNP in Table 3. The first column lists the region ID as presented in Table 1. The | | | | | | | | | | | | | | | | | | | |
| Haplotype column lists the specific nucleotides for the individual SNP alleles contributing to the haplotype reported. The Case and Control | | | | | | | | | | | | | | | | | | | |
| columns correspond to the numbers of cases and controls, respectively, containing the haplotype variant noted in the Haplotype column. The | | | | | | | | | | | | | | | | | | | |
| Total Case and Total Control columns list the total numbers of cases and controls for which genotype data was available for the haplotype | | | | | | | | | | | | | | | | | | | |
| in question, The RR column gives the relative risk for each particular haplotype. The remainder of the columns lists the SeqIDs for the SNPs | | | | | | | | | | | | | | | | | | | |
| contributing to the haplotype and their relative location with respect to the central marker. The Central marker (0) column lists the SeqID for | | | | | | | | | | | | | | | | | | | |
| the central marker on which the haplotype is based. Flanking markers are identified by minus (−) or plus (+) signs to indicate location of | | | | | | | | | | | | | | | | | | | |
| flanking SNPs. | | | | | | | | | | | | | | | | | | | |
| Re- | | | | | Total | | % | | Central | Central | Central | Central | Central | Central | Central | Central | Central | Central | Central |
| gion | | | Con- | Total | Con- | % | Con- | | marker | marker | marker | marker | marker | marker | marker | marker | marker | marker | marker |
| ID | Haplotype | Case | trol | Case | trol | Case | trol | RR | (−5) | (−4) | (−3) | (−2) | (−1) | (0) | (+1) | (+2) | (+3) | (+4) | (+5) |
| 1 | CTTG | 51 | 81 | 1258 | 1262 | 0.04 | 0.06 | 0.62 | | | | 8357 | 4850 | 4853 | 8358 | 4858 | | | |
| 1 | A | 820 | 767 | 1258 | 1262 | 0.65 | 0.61 | 1.21 | | | | | | 4858 | | | | | |
| 1 | G | 438 | 495 | 1258 | 1262 | 0.35 | 0.39 | 0.83 | | | | | | 4858 | | | | | |
| 1 | GAT | 125 | 166 | 1258 | 1262 | 0.10 | 0.13 | 0.73 | | | | | 4858 | 4863 | 8359 | | | | |
| 2 | A | 855 | 799 | 1272 | 1266 | 0.67 | 0.63 | 1.20 | | | | | | 4917 | | | | | |
| 2 | T | 417 | 467 | 1272 | 1266 | 0.33 | 0.37 | 0.83 | | | | | | 4917 | | | | | |
| 2 | TCG | 2 | 11 | 1272 | 1266 | 0.00 | 0.01 | 0.18 | | | | | 4917 | 8360 | 8361 | | | | |
| 3 | ACCGG | 27 | 11 | 1270 | 1266 | 0.02 | 0.01 | 2.48 | | | | 8362 | 4971 | 4972 | 4973 | 4975 | | | |
| 3 | CGG | 30 | 11 | 1270 | 1266 | 0.02 | 0.01 | 2.76 | | | | | 4972 | 4973 | 4975 | | | | |
| 3 | A | 1178 | 1214 | 1270 | 1266 | 0.93 | 0.96 | 0.55 | | | | | | 4975 | | | | | |
| 3 | G | 92 | 52 | 1270 | 1266 | 0.07 | 0.04 | 1.82 | | | | | | 4975 | | | | | |
| 3 | GGG | 91 | 52 | 1270 | 1266 | 0.07 | 0.04 | 1.80 | | | | | 4975 | 4977 | 8363 | | | | |
| 3 | AAGTT | 107 | 137 | 1270 | 1266 | 0.08 | 0.11 | 0.76 | | | | 4975 | 4977 | 8363 | 4978 | 4980 | | | |
| 3 | GGGTG | 89 | 51 | 1270 | 1266 | 0.07 | 0.04 | 1.80 | | | | 4975 | 4977 | 8363 | 4978 | 4980 | | | |
| 6 | T | 871 | 930 | 1266 | 1260 | 0.69 | 0.74 | 0.78 | | | | | | 5118 | | | | | |
| 6 | C | 395 | 330 | 1266 | 1260 | 0.31 | 0.26 | 1.28 | | | | | | 5118 | | | | | |
| 6 | CAG | 382 | 315 | 1266 | 1260 | 0.30 | 0.25 | 1.30 | | | | | 5118 | 5119 | 8375 | | | | |
| 6 | A | 553 | 617 | 1266 | 1260 | 0.44 | 0.49 | 0.81 | | | | | | 8375 | | | | | |
| 6 | G | 713 | 643 | 1266 | 1260 | 0.56 | 0.51 | 1.24 | | | | | | 8375 | | | | | |
| 6 | T | 609 | 683 | 1266 | 1260 | 0.48 | 0.54 | 0.78 | | | | | | 5120 | | | | | |
| 6 | C | 657 | 577 | 1266 | 1260 | 0.52 | 0.46 | 1.28 | | | | | | 5120 | | | | | |
| 6 | C | 403 | 470 | 1266 | 1260 | 0.32 | 0.37 | 0.78 | | | | | | 5122 | | | | | |
| 6 | T | 863 | 790 | 1266 | 1260 | 0.68 | 0.63 | 1.27 | | | | | | 5122 | | | | | |
| 6 | C | 385 | 462 | 1266 | 1260 | 0.30 | 0.37 | 0.75 | | | | | | 5123 | | | | | |
| 6 | A | 881 | 798 | 1266 | 1260 | 0.70 | 0.63 | 1.32 | | | | | | 5123 | | | | | |
| 6 | AAG | 612 | 544 | 1266 | 1260 | 0.48 | 0.43 | 1.23 | | | | | 5123 | 5124 | 8376 | | | | |
| 6 | CAG | 200 | 247 | 1266 | 1260 | 0.16 | 0.20 | 0.77 | | | | | 5123 | 5124 | 8376 | | | | |
| 6 | AAGCT | 490 | 429 | 1266 | 1260 | 0.39 | 0.34 | 1.22 | | | | 5123 | 5124 | 8376 | 5127 | 8377 | | | |
| 6 | CAGTC | 123 | 162 | 1266 | 1260 | 0.10 | 0.13 | 0.73 | | | | 5123 | 5124 | 8376 | 5127 | 8377 | | | |
| 6 | T | 419 | 466 | 1266 | 1260 | 0.33 | 0.37 | 0.84 | | | | | | 5127 | | | | | |
| 6 | C | 847 | 794 | 1266 | 1260 | 0.67 | 0.63 | 1.19 | | | | | | 5127 | | | | | |
| 6 | C | 435 | 482 | 1266 | 1260 | 0.34 | 0.38 | 0.84 | | | | | | 8377 | | | | | |
| 6 | T | 831 | 778 | 1266 | 1260 | 0.66 | 0.62 | 1.18 | | | | | | 8377 | | | | | |
| 7 | T | 1112 | 1146 | 1268 | 1264 | 0.88 | 0.91 | 0.73 | | | | | | 5240 | | | | | |
| 7 | C | 156 | 118 | 1268 | 1264 | 0.12 | 0.09 | 1.36 | | | | | | 5240 | | | | | |
| 7 | A | 1099 | 1130 | 1268 | 1264 | 0.87 | 0.89 | 0.77 | | | | | | 5241 | | | | | |
| 7 | C | 169 | 134 | 1268 | 1264 | 0.13 | 0.11 | 1.30 | | | | | | 5241 | | | | | |
| 8 | CTAGA | 257 | 308 | 1262 | 1260 | 0.20 | 0.24 | 0.79 | | | | 5309 | 5312 | 5313 | 8382 | 8383 | | | |
| 8 | AGG | 863 | 803 | 1262 | 1260 | 0.68 | 0.64 | 1.23 | | | | | 5313 | 8382 | 8383 | | | | |
| 8 | AGA | 257 | 308 | 1262 | 1260 | 0.20 | 0.24 | 0.79 | | | | | 5313 | 8382 | 8383 | | | | |
| 8 | G | 863 | 803 | 1262 | 1260 | 0.68 | 0.64 | 1.23 | | | | | | 8383 | | | | | |
| 8 | A | 399 | 457 | 1262 | 1260 | 0.32 | 0.36 | 0.81 | | | | | | 8383 | | | | | |
| 8 | AAC | 221 | 265 | 1262 | 1260 | 0.18 | 0.21 | 0.80 | | | | | 8383 | 8384 | 8385 | | | | |
| 9 | GGA | 336 | 287 | 1270 | 1262 | 0.26 | 0.23 | 1.22 | | | | | 5370 | 5371 | 5372 | | | | |
| 9 | ATG | 407 | 470 | 1270 | 1262 | 0.32 | 0.37 | 0.79 | | | | | 5370 | 5371 | 5372 | | | | |
| 9 | A | 861 | 789 | 1270 | 1262 | 0.68 | 0.63 | 1.26 | | | | | | 5372 | | | | | |
| 9 | G | 409 | 473 | 1270 | 1262 | 0.32 | 0.37 | 0.79 | | | | | | 5372 | | | | | |
| 9 | ATA | 489 | 426 | 1270 | 1262 | 0.39 | 0.34 | 1.23 | | | | | 5372 | 5374 | 8387 | | | | |
| 9 | GTA | 408 | 473 | 1270 | 1262 | 0.32 | 0.37 | 0.79 | | | | | 5372 | 5374 | 8387 | | | | |
| 10 | CGC | 368 | 421 | 1266 | 1254 | 0.29 | 0.34 | 0.81 | | | | | 8388 | 5442 | 5443 | | | | |
| 10 | CAT | 538 | 478 | 1266 | 1254 | 0.42 | 0.38 | 1.20 | | | | | 8388 | 5442 | 5443 | | | | |
| 10 | CGC | 714 | 756 | 1266 | 1254 | 0.56 | 0.60 | 0.85 | | | | | 5442 | 5443 | 5444 | | | | |
| 10 | ATT | 511 | 435 | 1266 | 1254 | 0.40 | 0.35 | 1.27 | | | | | 5442 | 5443 | 5444 | | | | |
| 10 | ATG | 27 | 43 | 1266 | 1254 | 0.02 | 0.03 | 0.61 | | | | | 5442 | 5443 | 5444 | | | | |
| 10 | CGCGGAC | 365 | 419 | 1266 | 1254 | 0.29 | 0.33 | 0.81 | | | 8388 | 5442 | 5443 | 5444 | 5445 | 5446 | 5448 | | |
| 10 | CATTACT | 472 | 387 | 1266 | 1254 | 0.37 | 0.31 | 1.33 | | | 8388 | 5442 | 5443 | 5444 | 5445 | 5446 | 5448 | | |
| 10 | CATGGAC | 27 | 43 | 1266 | 1254 | 0.02 | 0.03 | 0.61 | | | 8388 | 5442 | 5443 | 5444 | 5445 | 5446 | 5448 | | |
| 10 | GCGGACT | 390 | 455 | 1266 | 1254 | 0.31 | 0.36 | 0.78 | | | 5442 | 5443 | 5444 | 5445 | 5446 | 5448 | 8389 | | |
| 10 | ATTACTC | 472 | 387 | 1266 | 1254 | 0.37 | 0.31 | 1.33 | | | 5442 | 5443 | 5444 | 5445 | 5446 | 5448 | 8389 | | |
| 10 | ATGGACT | 26 | 43 | 1266 | 1254 | 0.02 | 0.03 | 0.59 | | | 5442 | 5443 | 5444 | 5445 | 5446 | 5448 | 8389 | | |
| 10 | GGACT | 416 | 498 | 1266 | 1254 | 0.33 | 0.40 | 0.74 | | | | 5444 | 5445 | 5446 | 5448 | 8389 | | | |
| 10 | TACTC | 472 | 387 | 1266 | 1254 | 0.37 | 0.31 | 1.33 | | | | 5444 | 5445 | 5446 | 5448 | 8389 | | | |
| 10 | ACT | 441 | 522 | 1266 | 1254 | 0.35 | 0.42 | 0.75 | | | | | 5446 | 5448 | 8389 | | | | |
| 10 | CTC | 477 | 389 | 1266 | 1254 | 0.38 | 0.31 | 1.34 | | | | | 5446 | 5448 | 8389 | | | | |
| 10 | ATC | 7 | 17 | 1266 | 1254 | 0.01 | 0.01 | 0.40 | | | | | 5446 | 5448 | 8389 | | | | |
| 10 | C | 825 | 732 | 1266 | 1254 | 0.65 | 0.58 | 1.33 | | | | | | 8389 | | | | | |
| 10 | T | 441 | 522 | 1266 | 1254 | 0.35 | 0.42 | 0.75 | | | | | | 8389 | | | | | |
| 10 | T | 879 | 801 | 1266 | 1254 | 0.69 | 0.64 | 1.28 | | | | | | 8390 | | | | | |
| 10 | C | 387 | 453 | 1266 | 1254 | 0.31 | 0.36 | 0.78 | | | | | | 8390 | | | | | |
| 12 | GGG | 31 | 61 | 1264 | 1264 | 0.02 | 0.05 | 0.50 | | | | | 5602 | 5603 | 5605 | | | | |
| 12 | GCATT | 294 | 245 | 1264 | 1264 | 0.23 | 0.19 | 1.26 | | | | 5602 | 5603 | 5605 | 5606 | 5607 | | | |
| 12 | GGGCC | 28 | 52 | 1264 | 1264 | 0.02 | 0.04 | 0.53 | | | | 5602 | 5603 | 5605 | 5606 | 5607 | | | |
| 12 | GCATTAG | 294 | 245 | 1264 | 1264 | 0.23 | 0.19 | 1.26 | | | 5602 | 5603 | 5605 | 5606 | 5607 | 5608 | 5609 | | |
| 12 | GGGCCAG | 23 | 47 | 1264 | 1264 | 0.02 | 0.04 | 0.48 | | | 5602 | 5603 | 5605 | 5606 | 5607 | 5608 | 5609 | | |
| 12 | T | 422 | 352 | 1264 | 1264 | 0.33 | 0.28 | 1.30 | | | | | | 5607 | | | | | |
| 12 | C | 842 | 912 | 1264 | 1264 | 0.67 | 0.72 | 0.77 | | | | | | 5607 | | | | | |
| 12 | TAG | 419 | 348 | 1264 | 1264 | 0.33 | 0.28 | 1.31 | | | | | 5607 | 5608 | 5609 | | | | |
| 12 | CAG | 581 | 638 | 1264 | 1264 | 0.45 | 0.50 | 0.83 | | | | | 5607 | 5608 | 5609 | | | | |
| 12 | TTAGTCG | 208 | 155 | 1264 | 1264 | 0.16 | 0.12 | 1.41 | | | 5606 | 5607 | 5608 | 5609 | 5611 | 5612 | 5613 | | |
| 12 | TCAGTCG | 8 | 20 | 1264 | 1264 | 0.01 | 0.02 | 0.40 | | | 5606 | 5607 | 5608 | 5609 | 5611 | 5612 | 5613 | | |
| 13 | TAA | 842 | 766 | 1258 | 1236 | 0.67 | 0.62 | 1.24 | | | | | 5728 | 5729 | 5730 | | | | |
| 13 | CCG | 162 | 195 | 1258 | 1236 | 0.13 | 0.16 | 0.79 | | | | | 5728 | 5729 | 5730 | | | | |
| 13 | A | 860 | 773 | 1258 | 1236 | 0.68 | 0.63 | 1.29 | | | | | | 5730 | | | | | |
| 13 | G | 398 | 463 | 1258 | 1236 | 0.32 | 0.37 | 0.77 | | | | | | 5730 | | | | | |
| 13 | C | 940 | 868 | 1258 | 1236 | 0.75 | 0.70 | 1.25 | | | | | | 8392 | | | | | |
| 13 | A | 318 | 368 | 1258 | 1236 | 0.25 | 0.30 | 0.80 | | | | | | 8392 | | | | | |
| 13 | TAACCTCTT | 28 | 10 | 1258 | 1236 | 0.02 | 0.01 | 2.79 | | 5728 | 5729 | 5730 | 8392 | 5732 | 5733 | 5734 | 5735 | 5736 | |
| 13 | AACCTCTTC | 27 | 10 | 1258 | 1236 | 0.02 | 0.01 | 2.69 | | 5729 | 5730 | 8392 | 5732 | 5733 | 5734 | 5735 | 5736 | 8393 | |
| 13 | CTCTT | 30 | 12 | 1258 | 1236 | 0.02 | 0.01 | 2.49 | | | | 5732 | 5733 | 5734 | 5735 | 5736 | | | |
| 13 | TTCAT | 82 | 58 | 1258 | 1236 | 0.07 | 0.05 | 1.42 | | | | 5732 | 5733 | 5734 | 5735 | 5736 | | | |
| 13 | CTT | 36 | 16 | 1258 | 1236 | 0.03 | 0.01 | 2.25 | | | | | 5734 | 5735 | 5736 | | | | |
| 14 | T | 804 | 869 | 1270 | 1254 | 0.63 | 0.69 | 0.76 | | | | | | 5803 | | | | | |
| 14 | G | 466 | 385 | 1270 | 1254 | 0.37 | 0.31 | 1.31 | | | | | | 5803 | | | | | |
| 14 | C | 833 | 901 | 1270 | 1254 | 0.66 | 0.72 | 0.75 | | | | | | 8394 | | | | | |
| 14 | T | 437 | 353 | 1270 | 1254 | 0.34 | 0.28 | 1.34 | | | | | | 8394 | | | | | |
| 14 | C | 804 | 872 | 1270 | 1254 | 0.63 | 0.70 | 0.76 | | | | | | 8395 | | | | | |
| 14 | A | 466 | 382 | 1270 | 1254 | 0.37 | 0.30 | 1.32 | | | | | | 8395 | | | | | |
| 14 | C | 884 | 948 | 1270 | 1254 | 0.70 | 0.76 | 0.74 | | | | | | 5811 | | | | | |
| 14 | T | 386 | 306 | 1270 | 1254 | 0.30 | 0.24 | 1.35 | | | | | | 5811 | | | | | |
| 14 | T | 874 | 932 | 1270 | 1254 | 0.69 | 0.74 | 0.76 | | | | | | 5815 | | | | | |
| 14 | C | 396 | 322 | 1270 | 1254 | 0.31 | 0.26 | 1.31 | | | | | | 5815 | | | | | |
| 14 | A | 901 | 970 | 1270 | 1254 | 0.71 | 0.77 | 0.71 | | | | | | 5816 | | | | | |
| 14 | G | 369 | 284 | 1270 | 1254 | 0.29 | 0.23 | 1.40 | | | | | | 5816 | | | | | |
| 14 | A | 881 | 936 | 1270 | 1254 | 0.69 | 0.75 | 0.77 | | | | | | 5817 | | | | | |
| 14 | G | 389 | 318 | 1270 | 1254 | 0.31 | 0.25 | 1.30 | | | | | | 5817 | | | | | |
| 14 | AAT | 876 | 930 | 1270 | 1254 | 0.69 | 0.74 | 0.77 | | | | | 5817 | 8396 | 8397 | | | | |
| 14 | GCC | 364 | 281 | 1270 | 1254 | 0.29 | 0.22 | 1.39 | | | | | 5817 | 8396 | 8397 | | | | |
| 14 | T | 906 | 973 | 1270 | 1254 | 0.71 | 0.78 | 0.72 | | | | | | 8397 | | | | | |
| 14 | C | 364 | 281 | 1270 | 1254 | 0.29 | 0.22 | 1.39 | | | | | | 8397 | | | | | |
| 15 | CTCTA | 157 | 116 | 1272 | 1256 | 0.12 | 0.09 | 1.38 | | | | 8398 | 5870 | 8399 | 5874 | 5880 | | | |
| 15 | CTA | 191 | 149 | 1272 | 1256 | 0.15 | 0.12 | 1.31 | | | | | 8399 | 5874 | 5880 | | | | |
| 15 | G | 1076 | 1106 | 1272 | 1256 | 0.85 | 0.88 | 0.74 | | | | | | 5880 | | | | | |
| 15 | A | 196 | 150 | 1272 | 1256 | 0.15 | 0.12 | 1.34 | | | | | | 5880 | | | | | |
| 15 | A | 1074 | 1104 | 1272 | 1256 | 0.84 | 0.88 | 0.75 | | | | | | 5882 | | | | | |
| 15 | C | 198 | 152 | 1272 | 1256 | 0.16 | 0.12 | 1.34 | | | | | | 5882 | | | | | |
| 15 | CTA | 185 | 144 | 1272 | 1256 | 0.15 | 0.11 | 1.31 | | | | | 5882 | 5885 | 5889 | | | | |
| 15 | ATG | 48 | 73 | 1272 | 1256 | 0.04 | 0.06 | 0.64 | | | | | 5882 | 5885 | 5889 | | | | |
| 15 | A | 1188 | 1143 | 1272 | 1256 | 0.93 | 0.91 | 1.40 | | | | | | 5889 | | | | | |
| 15 | G | 84 | 113 | 1272 | 1256 | 0.07 | 0.09 | 0.72 | | | | | | 5889 | | | | | |
| 16 | T | 654 | 587 | 1264 | 1266 | 0.52 | 0.46 | 1.24 | | | | | | 5917 | | | | | |
| 16 | A | 610 | 679 | 1264 | 1266 | 0.48 | 0.54 | 0.81 | | | | | | 5917 | | | | | |
| 16 | TAC | 642 | 579 | 1264 | 1266 | 0.51 | 0.46 | 1.22 | | | | | 5917 | 5918 | 5921 | | | | |
| 16 | AAC | 446 | 525 | 1264 | 1266 | 0.35 | 0.41 | 0.77 | | | | | 5917 | 5918 | 5921 | | | | |
| 16 | TACTT | 636 | 575 | 1264 | 1266 | 0.50 | 0.45 | 1.22 | | | | 5917 | 5918 | 5921 | 5922 | 5923 | | | |
| 16 | AACTT | 163 | 220 | 1264 | 1266 | 0.13 | 0.17 | 0.70 | | | | 5917 | 5918 | 5921 | 5922 | 5923 | | | |
| 16 | TACTTGA | 635 | 573 | 1264 | 1266 | 0.50 | 0.45 | 1.22 | | | 5917 | 5918 | 5921 | 5922 | 5923 | 5924 | 5925 | | |
| 16 | AACTTGA | 163 | 218 | 1264 | 1266 | 0.13 | 0.17 | 0.71 | | | 5917 | 5918 | 5921 | 5922 | 5923 | 5924 | 5925 | | |
| 16 | TACTTGATG | 565 | 509 | 1264 | 1266 | 0.45 | 0.40 | 1.20 | | 5917 | 5918 | 5921 | 5922 | 5923 | 5924 | 5925 | 5928 | 5930 | |
| 16 | AACTTGACG | 15 | 38 | 1264 | 1266 | 0.01 | 0.03 | 0.39 | | 5917 | 5918 | 5921 | 5922 | 5923 | 5924 | 5925 | 5928 | 5930 | |
| 16 | TACTTGATGTC | 537 | 486 | 1264 | 1266 | 0.42 | 0.38 | 1.19 | 5917 | 5918 | 5921 | 5922 | 5923 | 5924 | 5925 | 5928 | 5930 | 8401 | 5933 |
| 16 | AACTTGATGTT | 94 | 126 | 1264 | 1266 | 0.07 | 0.10 | 0.73 | 5917 | 5918 | 5921 | 5922 | 5923 | 5924 | 5925 | 5928 | 5930 | 8401 | 5933 |
| 16 | AACTTGACGTT | 15 | 36 | 1264 | 1266 | 0.01 | 0.03 | 0.41 | 5917 | 5918 | 5921 | 5922 | 5923 | 5924 | 5925 | 5928 | 5930 | 8401 | 5933 |
| 16 | CTTGATGTC | 589 | 534 | 1264 | 1266 | 0.47 | 0.42 | 1.20 | | 5921 | 5922 | 5923 | 5924 | 5925 | 5928 | 5930 | 8401 | 5933 | |
| 16 | TGATGTC | 701 | 630 | 1264 | 1266 | 0.55 | 0.50 | 1.26 | | | 5923 | 5924 | 5925 | 5928 | 5930 | 8401 | 5933 | | |
| 16 | ATGTC | 706 | 632 | 1264 | 1266 | 0.56 | 0.50 | 1.27 | | | | 5925 | 5928 | 5930 | 8401 | 5933 | | | |
| 16 | GTC | 706 | 634 | 1264 | 1266 | 0.56 | 0.50 | 1.26 | | | | | 5930 | 8401 | 5933 | | | | |
| 16 | GTT | 192 | 241 | 1264 | 1266 | 0.15 | 0.19 | 0.76 | | | | | 5930 | 8401 | 5933 | | | | |
| 16 | C | 779 | 707 | 1264 | 1266 | 0.62 | 0.56 | 1.27 | | | | | | 5933 | | | | | |
| 16 | T | 485 | 559 | 1264 | 1266 | 0.38 | 0.44 | 0.79 | | | | | | 5933 | | | | | |
| 17 | GGTCT | 259 | 205 | 1270 | 1264 | 0.20 | 0.16 | 1.32 | | | | 8403 | 8404 | 5973 | 8405 | 5980 | | | |
| 17 | G | 962 | 1006 | 1270 | 1264 | 0.76 | 0.80 | 0.80 | | | | | | 5980 | | | | | |
| 17 | T | 308 | 258 | 1270 | 1264 | 0.24 | 0.20 | 1.25 | | | | | | 5980 | | | | | |
| 17 | TGC | 240 | 188 | 1270 | 1264 | 0.19 | 0.15 | 1.33 | | | | | 5980 | 8406 | 8407 | | | | |
| 17 | A | 930 | 975 | 1270 | 1264 | 0.73 | 0.77 | 0.81 | | | | | | 8407 | | | | | |
| 17 | C | 340 | 289 | 1270 | 1264 | 0.27 | 0.23 | 1.23 | | | | | | 8407 | | | | | |
| 18 | ACGGA | 49 | 71 | 1270 | 1262 | 0.04 | 0.06 | 0.67 | | | | 6062 | 8409 | 8410 | 8411 | 6070 | | | |
| 18 | CGGAG | 45 | 70 | 1270 | 1262 | 0.04 | 0.06 | 0.63 | | | | 8409 | 8410 | 8411 | 6070 | 6071 | | | |
| 18 | G | 1200 | 1158 | 1270 | 1262 | 0.94 | 0.92 | 1.54 | | | | | | 6070 | | | | | |
| 18 | A | 70 | 104 | 1270 | 1262 | 0.06 | 0.08 | 0.65 | | | | | | 6070 | | | | | |
| 18 | C | 1111 | 1046 | 1270 | 1262 | 0.87 | 0.83 | 1.44 | | | | | | 6071 | | | | | |
| 18 | G | 159 | 216 | 1270 | 1262 | 0.13 | 0.17 | 0.69 | | | | | | 6071 | | | | | |
| 18 | A | 1079 | 1005 | 1270 | 1262 | 0.85 | 0.80 | 1.44 | | | | | | 6073 | | | | | |
| 18 | G | 191 | 257 | 1270 | 1262 | 0.15 | 0.20 | 0.69 | | | | | | 6073 | | | | | |
| 18 | G | 1081 | 1007 | 1270 | 1262 | 0.85 | 0.80 | 1.45 | | | | | | 8412 | | | | | |
| 18 | C | 189 | 255 | 1270 | 1262 | 0.15 | 0.20 | 0.69 | | | | | | 8412 | | | | | |
| 19 | AAACA | 622 | 677 | 1264 | 1264 | 0.49 | 0.54 | 0.84 | | | | 6096 | 8414 | 8415 | 8416 | 8417 | | | |
| 19 | TAACA | 2 | 10 | 1264 | 1264 | 0.00 | 0.01 | 0.20 | | | | 6096 | 8414 | 8415 | 8416 | 8417 | | | |
| 19 | AACAC | 622 | 680 | 1264 | 1264 | 0.49 | 0.54 | 0.83 | | | | 8414 | 8415 | 8416 | 8417 | 6098 | | | |
| 19 | TGCTT | 244 | 201 | 1264 | 1264 | 0.19 | 0.16 | 1.27 | | | | 8414 | 8415 | 8416 | 8417 | 6098 | | | |
| 19 | A | 624 | 688 | 1264 | 1264 | 0.49 | 0.54 | 0.82 | | | | | | 8417 | | | | | |
| 19 | T | 640 | 576 | 1264 | 1264 | 0.51 | 0.46 | 1.23 | | | | | | 8417 | | | | | |
| 19 | ACA | 622 | 681 | 1264 | 1264 | 0.49 | 0.54 | 0.83 | | | | | 8417 | 6098 | 6100 | | | | |
| 19 | ACACC | 618 | 670 | 1264 | 1264 | 0.49 | 0.53 | 0.85 | | | | 8417 | 6098 | 6100 | 8418 | 6101 | | | |
| 19 | CACCA | 619 | 672 | 1264 | 1264 | 0.49 | 0.53 | 0.85 | | | | 6098 | 6100 | 8418 | 6101 | 6102 | | | |
| 19 | C | 621 | 672 | 1264 | 1264 | 0.49 | 0.53 | 0.85 | | | | | | 6101 | | | | | |
| 19 | G | 643 | 592 | 1264 | 1264 | 0.51 | 0.47 | 1.18 | | | | | | 6101 | | | | | |
| 20 | CGG | 521 | 452 | 1270 | 1262 | 0.41 | 0.36 | 1.25 | | | | | 6172 | 6175 | 6176 | | | | |
| 20 | G | 855 | 778 | 1270 | 1262 | 0.67 | 0.62 | 1.28 | | | | | | 6176 | | | | | |
| 20 | A | 415 | 484 | 1270 | 1262 | 0.33 | 0.38 | 0.78 | | | | | | 6176 | | | | | |
| 20 | GAT | 809 | 740 | 1270 | 1262 | 0.64 | 0.59 | 1.24 | | | | | 6176 | 6179 | 6182 | | | | |
| 20 | AGT | 124 | 167 | 1270 | 1262 | 0.10 | 0.13 | 0.71 | | | | | 6176 | 6179 | 6182 | | | | |
| 20 | AAC | 13 | 27 | 1270 | 1262 | 0.01 | 0.02 | 0.47 | | | | | 6176 | 6179 | 6182 | | | | |
| 20 | GATTC | 739 | 668 | 1270 | 1262 | 0.58 | 0.53 | 1.24 | | | | 6176 | 6179 | 6182 | 6183 | 6184 | | | |
| 20 | AGTCT | 106 | 144 | 1270 | 1262 | 0.08 | 0.11 | 0.71 | | | | 6176 | 6179 | 6182 | 6183 | 6184 | | | |
| 20 | AACCT | 13 | 27 | 1270 | 1262 | 0.01 | 0.02 | 0.47 | | | | 6176 | 6179 | 6182 | 6183 | 6184 | | | |
| 20 | T | 778 | 715 | 1270 | 1262 | 0.61 | 0.57 | 1.21 | | | | | | 6183 | | | | | |
| 20 | C | 492 | 547 | 1270 | 1262 | 0.39 | 0.43 | 0.83 | | | | | | 6183 | | | | | |
| 20 | TCT | 778 | 715 | 1270 | 1262 | 0.61 | 0.57 | 1.21 | | | | | 6183 | 6184 | 6185 | | | | |
| 20 | CTG | 492 | 547 | 1270 | 1262 | 0.39 | 0.43 | 0.83 | | | | | 6183 | 6184 | 6185 | | | | |
| 20 | T | 778 | 715 | 1270 | 1262 | 0.61 | 0.57 | 1.21 | | | | | | 6185 | | | | | |
| 20 | G | 492 | 547 | 1270 | 1262 | 0.39 | 0.43 | 0.83 | | | | | | 6185 | | | | | |
| 20 | TCTGCCG | 523 | 440 | 1270 | 1262 | 0.41 | 0.35 | 1.31 | | | 6183 | 6184 | 6185 | 6186 | 6188 | 6189 | 6190 | | |
| 20 | TGCCG | 523 | 440 | 1270 | 1262 | 0.41 | 0.35 | 1.31 | | | | 6185 | 6186 | 6188 | 6189 | 6190 | | | |
| 21 | TCG | 111 | 163 | 1268 | 1266 | 0.09 | 0.13 | 0.65 | | | | | 6252 | 6254 | 6255 | | | | |
| 21 | CAC | 686 | 624 | 1268 | 1266 | 0.54 | 0.49 | 1.21 | | | | | 6254 | 6255 | 6256 | | | | |
| 21 | CGC | 217 | 278 | 1268 | 1266 | 0.17 | 0.22 | 0.73 | | | | | 6254 | 6255 | 6256 | | | | |
| 21 | ACT | 629 | 564 | 1268 | 1266 | 0.50 | 0.45 | 1.23 | | | | | 6255 | 6256 | 6257 | | | | |
| 21 | GCC | 216 | 278 | 1268 | 1266 | 0.17 | 0.22 | 0.73 | | | | | 6255 | 6256 | 6257 | | | | |
| 21 | T | 994 | 924 | 1268 | 1266 | 0.78 | 0.73 | 1.34 | | | | | | 6257 | | | | | |
| 21 | C | 274 | 342 | 1268 | 1266 | 0.22 | 0.27 | 0.74 | | | | | | 6257 | | | | | |
| 21 | CACTTGGAT | 558 | 497 | 1268 | 1266 | 0.44 | 0.39 | 1.22 | | 6254 | 6255 | 6256 | 6257 | 6258 | 6259 | 6260 | 6262 | 6264 | |
| 21 | CGCCTACGT | 43 | 64 | 1268 | 1266 | 0.03 | 0.05 | 0.66 | | 6254 | 6255 | 6256 | 6257 | 6258 | 6259 | 6260 | 6262 | 6264 | |
| 21 | CACTTGGGC | 14 | 34 | 1268 | 1266 | 0.01 | 0.03 | 0.40 | | 6254 | 6255 | 6256 | 6257 | 6258 | 6259 | 6260 | 6262 | 6264 | |
| 21 | CACTTGGAC | 8 | 1 | 1268 | 1266 | 0.01 | 0.00 | 8.03 | | 6254 | 6255 | 6256 | 6257 | 6258 | 6259 | 6260 | 6262 | 6264 | |
| 21 | CTTGGAT | 560 | 497 | 1268 | 1266 | 0.44 | 0.39 | 1.22 | | | 6256 | 6257 | 6258 | 6259 | 6260 | 6262 | 6264 | | |
| 21 | CCGGCGT | 33 | 52 | 1268 | 1266 | 0.03 | 0.04 | 0.62 | | | 6256 | 6257 | 6258 | 6259 | 6260 | 6262 | 6264 | | |
| 21 | CCTACGT | 43 | 65 | 1268 | 1266 | 0.03 | 0.05 | 0.65 | | | 6256 | 6257 | 6258 | 6259 | 6260 | 6262 | 6264 | | |
| 21 | CTTGGGC | 14 | 34 | 1268 | 1266 | 0.01 | 0.03 | 0.40 | | | 6256 | 6257 | 6258 | 6259 | 6260 | 6262 | 6264 | | |
| 21 | CTTGGAC | 8 | 1 | 1268 | 1266 | 0.01 | 0.00 | 8.03 | | | 6256 | 6257 | 6258 | 6259 | 6260 | 6262 | 6264 | | |
| 21 | G | 1154 | 1097 | 1268 | 1266 | 0.91 | 0.87 | 1.56 | | | | | | 6260 | | | | | |
| 21 | C | 114 | 169 | 1268 | 1266 | 0.09 | 0.13 | 0.64 | | | | | | 6260 | | | | | |
| 21 | A | 1085 | 1014 | 1268 | 1266 | 0.86 | 0.80 | 1.47 | | | | | | 6262 | | | | | |
| 21 | G | 183 | 252 | 1268 | 1266 | 0.14 | 0.20 | 0.68 | | | | | | 6262 | | | | | |
| 23 | GCG | 93 | 132 | 1270 | 1262 | 0.07 | 0.10 | 0.68 | | | | | 8428 | 8429 | 6409 | | | | |
| 23 | GTA | 191 | 152 | 1270 | 1262 | 0.15 | 0.12 | 1.29 | | | | | 8428 | 8429 | 6409 | | | | |
| 23 | A | 1167 | 1120 | 1270 | 1262 | 0.92 | 0.89 | 1.44 | | | | | | 6409 | | | | | |
| 23 | G | 103 | 142 | 1270 | 1262 | 0.08 | 0.11 | 0.70 | | | | | | 6409 | | | | | |
| 23 | C | 1158 | 1115 | 1270 | 1262 | 0.91 | 0.88 | 1.36 | | | | | | 8431 | | | | | |
| 23 | A | 112 | 147 | 1270 | 1262 | 0.09 | 0.12 | 0.73 | | | | | | 8431 | | | | | |
| 23 | CGAGACC | 96 | 135 | 1270 | 1262 | 0.08 | 0.11 | 0.68 | | | 8429 | 6409 | 8431 | 8432 | 6411 | 8434 | 8435 | | |
| 23 | TACGGCC | 178 | 134 | 1270 | 1262 | 0.14 | 0.11 | 1.37 | | | 8429 | 6409 | 8431 | 8432 | 6411 | 8434 | 8435 | | |
| 23 | ACC | 111 | 149 | 1270 | 1262 | 0.09 | 0.12 | 0.72 | | | | | 6411 | 8434 | 8435 | | | | |
| 24 | CAG | 416 | 477 | 1266 | 1264 | 0.33 | 0.38 | 0.81 | | | | | 8436 | 8437 | 6676 | | | | |
| 24 | CAC | 367 | 317 | 1266 | 1264 | 0.29 | 0.25 | 1.22 | | | | | 8436 | 8437 | 6676 | | | | |
| 24 | G | 833 | 878 | 1266 | 1264 | 0.66 | 0.69 | 0.85 | | | | | | 6676 | | | | | |
| 24 | C | 433 | 386 | 1266 | 1264 | 0.34 | 0.31 | 1.18 | | | | | | 6676 | | | | | |
| 24 | CAGTTTTGA | 27 | 47 | 1266 | 1264 | 0.02 | 0.04 | 0.56 | | 8436 | 8437 | 6676 | 8440 | 8441 | 8442 | 8443 | 6679 | 6680 | |
| 24 | CAGCGTTCA | 13 | 2 | 1266 | 1264 | 0.01 | 0.00 | 6.55 | | 8436 | 8437 | 6676 | 8440 | 8441 | 8442 | 8443 | 6679 | 6680 | |
| 24 | AAGCGTCCA | 26 | 9 | 1266 | 1264 | 0.02 | 0.01 | 2.92 | | 8436 | 8437 | 6676 | 8440 | 8441 | 8442 | 8443 | 6679 | 6680 | |
| 24 | CGTCG | 280 | 332 | 1266 | 1264 | 0.22 | 0.26 | 0.80 | | | | 8440 | 8441 | 8442 | 8443 | 6679 | | | |
| 24 | CGTTC | 17 | 2 | 1266 | 1264 | 0.01 | 0.00 | 8.59 | | | | 8440 | 8441 | 8442 | 8443 | 6679 | | | |
| 24 | CGTCC | 58 | 33 | 1266 | 1264 | 0.05 | 0.03 | 1.79 | | | | 8440 | 8441 | 8442 | 8443 | 6679 | | | |
| 24 | GTCGG | 92 | 122 | 1266 | 1264 | 0.07 | 0.10 | 0.73 | | | | 8441 | 8442 | 8443 | 6679 | 6680 | | | |
| 24 | GTTCA | 17 | 2 | 1266 | 1264 | 0.01 | 0.00 | 8.59 | | | | 8441 | 8442 | 8443 | 6679 | 6680 | | | |
| 24 | GTCCA | 56 | 29 | 1266 | 1264 | 0.04 | 0.02 | 1.97 | | | | 8441 | 8442 | 8443 | 6679 | 6680 | | | |
| 24 | G | 957 | 1015 | 1266 | 1264 | 0.76 | 0.80 | 0.76 | | | | | | 6679 | | | | | |
| 24 | C | 309 | 249 | 1266 | 1264 | 0.24 | 0.20 | 1.32 | | | | | | 6679 | | | | | |
| 24 | CAT | 281 | 227 | 1266 | 1264 | 0.22 | 0.18 | 1.30 | | | | | 6679 | 6680 | 8444 | | | | |
| 24 | CCATCAG | 57 | 29 | 1266 | 1264 | 0.05 | 0.02 | 2.01 | | | 8443 | 6679 | 6680 | 8444 | 6682 | 8445 | 8446 | | |
| 25 | C | 739 | 653 | 1260 | 1252 | 0.59 | 0.52 | 1.30 | | | | | | 8447 | | | | | |
| 25 | T | 521 | 599 | 1260 | 1252 | 0.41 | 0.48 | 0.77 | | | | | | 8447 | | | | | |
| 25 | CAA | 416 | 337 | 1260 | 1252 | 0.33 | 0.27 | 1.34 | | | | | 8447 | 6721 | 6723 | | | | |
| 25 | CAACC | 201 | 157 | 1260 | 1252 | 0.16 | 0.13 | 1.32 | | | | 8447 | 6721 | 6723 | 8448 | 6728 | | | |
| 25 | TAACG | 66 | 104 | 1260 | 1252 | 0.05 | 0.08 | 0.61 | | | | 8447 | 6721 | 6723 | 8448 | 6728 | | | |
| 25 | ACG | 139 | 174 | 1260 | 1252 | 0.11 | 0.14 | 0.77 | | | | | 6723 | 8448 | 6728 | | | | |
| 25 | C | 1056 | 1000 | 1260 | 1252 | 0.84 | 0.80 | 1.30 | | | | | | 6728 | | | | | |
| 25 | G | 204 | 252 | 1260 | 1252 | 0.16 | 0.20 | 0.77 | | | | | | 6728 | | | | | |
| 25 | GTG | 204 | 252 | 1260 | 1252 | 0.16 | 0.20 | 0.77 | | | | | 6728 | 6731 | 6732 | | | | |
| 25 | GTGTT | 166 | 223 | 1260 | 1252 | 0.13 | 0.18 | 0.70 | | | | 6728 | 6731 | 6732 | 6733 | 6734 | | | |
| 25 | GTT | 424 | 481 | 1260 | 1252 | 0.34 | 0.38 | 0.81 | | | | | 6732 | 6733 | 6734 | | | | |
| 26 | T | 865 | 916 | 1272 | 1264 | 0.68 | 0.72 | 0.81 | | | | | | 6762 | | | | | |
| 26 | G | 407 | 348 | 1272 | 1264 | 0.32 | 0.28 | 1.24 | | | | | | 6762 | | | | | |
| 26 | T | 857 | 803 | 1272 | 1264 | 0.67 | 0.64 | 1.19 | | | | | | 6765 | | | | | |
| 26 | C | 415 | 461 | 1272 | 1264 | 0.33 | 0.36 | 0.84 | | | | | | 6765 | | | | | |
| 26 | C | 911 | 842 | 1272 | 1264 | 0.72 | 0.67 | 1.26 | | | | | | 6767 | | | | | |
| 26 | T | 361 | 422 | 1272 | 1264 | 0.28 | 0.33 | 0.79 | | | | | | 6767 | | | | | |
| 26 | TTGTG | 13 | 26 | 1272 | 1264 | 0.01 | 0.02 | 0.49 | | | | 6766 | 6767 | 6769 | 6770 | 6772 | | | |
| 26 | CCCAC | 28 | 14 | 1272 | 1264 | 0.02 | 0.01 | 2.01 | | | | 6766 | 6767 | 6769 | 6770 | 6772 | | | |
| 28 | TGCTATG | 96 | 136 | 1266 | 1262 | 0.08 | 0.11 | 0.68 | | | 8450 | 8451 | 8452 | 8453 | 8454 | 6914 | 6915 | | |
| 28 | GCTATGG | 96 | 136 | 1266 | 1262 | 0.08 | 0.11 | 0.68 | | | 8451 | 8452 | 8453 | 8454 | 6914 | 6915 | 6916 | | |
| 28 | ATG | 96 | 137 | 1266 | 1262 | 0.08 | 0.11 | 0.67 | | | | | 8454 | 6914 | 6915 | | | | |
| 28 | A | 1170 | 1125 | 1266 | 1262 | 0.92 | 0.89 | 1.48 | | | | | | 6915 | | | | | |
| 28 | G | 96 | 137 | 1266 | 1262 | 0.08 | 0.11 | 0.67 | | | | | | 6915 | | | | | |
| 29 | TCGTG | 120 | 152 | 1264 | 1266 | 0.09 | 0.12 | 0.77 | | | | 8457 | 8458 | 8460 | 8461 | 6979 | | | |
| 29 | GTA | 854 | 794 | 1264 | 1266 | 0.68 | 0.63 | 1.24 | | | | | 8460 | 8461 | 6979 | | | | |
| 29 | GTG | 230 | 285 | 1264 | 1266 | 0.18 | 0.23 | 0.77 | | | | | 8460 | 8461 | 6979 | | | | |
| 29 | A | 1033 | 977 | 1264 | 1266 | 0.82 | 0.77 | 1.32 | | | | | | 6979 | | | | | |
| 29 | G | 231 | 289 | 1264 | 1266 | 0.18 | 0.23 | 0.76 | | | | | | 6979 | | | | | |
| 29 | GGC | 228 | 288 | 1264 | 1266 | 0.18 | 0.23 | 0.75 | | | | | 6979 | 6980 | 8463 | | | | |
| 30 | A | 1113 | 1078 | 1254 | 1260 | 0.89 | 0.86 | 1.33 | | | | | | 8465 | | | | | |
| 30 | T | 141 | 182 | 1254 | 1260 | 0.11 | 0.14 | 0.75 | | | | | | 8465 | | | | | |
| 30 | AAA | 811 | 747 | 1254 | 1260 | 0.65 | 0.59 | 1.26 | | | | | 8465 | 8466 | 8467 | | | | |
| 30 | TAC | 106 | 143 | 1254 | 1260 | 0.08 | 0.11 | 0.72 | | | | | 8465 | 8466 | 8467 | | | | |
| 30 | A | 1006 | 948 | 1254 | 1260 | 0.80 | 0.75 | 1.34 | | | | | | 8467 | | | | | |
| 30 | C | 248 | 312 | 1254 | 1260 | 0.20 | 0.25 | 0.75 | | | | | | 8467 | | | | | |
| 30 | ACT | 653 | 570 | 1254 | 1260 | 0.52 | 0.45 | 1.32 | | | | | 8467 | 7054 | 7056 | | | | |
| 30 | CCA | 197 | 262 | 1254 | 1260 | 0.16 | 0.21 | 0.71 | | | | | 8467 | 7054 | 7056 | | | | |
| 30 | T | 837 | 752 | 1254 | 1260 | 0.67 | 0.60 | 1.36 | | | | | | 7056 | | | | | |
| 30 | A | 417 | 508 | 1254 | 1260 | 0.33 | 0.40 | 0.74 | | | | | | 7056 | | | | | |
| 30 | C | 810 | 887 | 1254 | 1260 | 0.65 | 0.70 | 0.77 | | | | | | 7058 | | | | | |
| 30 | T | 444 | 373 | 1254 | 1260 | 0.35 | 0.30 | 1.30 | | | | | | 7058 | | | | | |
| 30 | CAC | 473 | 544 | 1254 | 1260 | 0.38 | 0.43 | 0.80 | | | | | 7058 | 7059 | 8468 | | | | |
| 30 | TAC | 442 | 371 | 1254 | 1260 | 0.35 | 0.29 | 1.30 | | | | | 7058 | 7059 | 8468 | | | | |
| 30 | ACA | 364 | 422 | 1254 | 1260 | 0.29 | 0.33 | 0.81 | | | | | 7059 | 8468 | 7061 | | | | |
| 30 | ACG | 551 | 493 | 1254 | 1260 | 0.44 | 0.39 | 1.22 | | | | | 7059 | 8468 | 7061 | | | | |
| 30 | G | 890 | 837 | 1254 | 1260 | 0.71 | 0.66 | 1.24 | | | | | | 7061 | | | | | |
| 30 | A | 364 | 423 | 1254 | 1260 | 0.29 | 0.34 | 0.81 | | | | | | 7061 | | | | | |
| 31 | GTC | 285 | 349 | 1270 | 1260 | 0.22 | 0.28 | 0.76 | | | | | 8472 | 7103 | 7106 | | | | |
| 31 | CTC | 532 | 475 | 1270 | 1260 | 0.42 | 0.38 | 1.19 | | | | | 7103 | 7106 | 8473 | | | | |
| 31 | TCT | 195 | 240 | 1270 | 1260 | 0.15 | 0.19 | 0.77 | | | | | 7103 | 7106 | 8473 | | | | |
| 31 | CCC | 13 | 28 | 1270 | 1260 | 0.01 | 0.02 | 0.46 | | | | | 7103 | 7106 | 8473 | | | | |
| 31 | C | 1048 | 997 | 1270 | 1260 | 0.83 | 0.79 | 1.25 | | | | | | 8473 | | | | | |
| 31 | T | 222 | 263 | 1270 | 1260 | 0.17 | 0.21 | 0.80 | | | | | | 8473 | | | | | |
| 31 | A | 882 | 814 | 1270 | 1260 | 0.69 | 0.65 | 1.25 | | | | | | 7108 | | | | | |
| 31 | C | 388 | 446 | 1270 | 1260 | 0.31 | 0.35 | 0.80 | | | | | | 7108 | | | | | |
| 32 | GGATATA | 39 | 19 | 1272 | 1264 | 0.03 | 0.02 | 2.07 | | | 8477 | 8478 | 8479 | 8480 | 8481 | 8482 | 8483 | | |
| 32 | ATATA | 40 | 22 | 1272 | 1264 | 0.03 | 0.02 | 1.83 | | | | 8479 | 8480 | 8481 | 8482 | 8483 | | | |
| 33 | CGCTT | 11 | 3 | 1270 | 1258 | 0.01 | 0.00 | 3.66 | | | | 8488 | 8489 | 8491 | 8492 | 8493 | | | |
| 33 | CGCTTTC | 11 | 3 | 1270 | 1258 | 0.01 | 0.00 | 3.66 | | | 8488 | 8489 | 8491 | 8492 | 8493 | 8494 | 7334 | | |
| 34 | C | 340 | 435 | 1272 | 1262 | 0.27 | 0.34 | 0.69 | | | | | | 8495 | | | | | |
| 34 | T | 932 | 827 | 1272 | 1262 | 0.73 | 0.66 | 1.44 | | | | | | 8495 | | | | | |
| 34 | A | 370 | 467 | 1272 | 1262 | 0.29 | 0.37 | 0.70 | | | | | | 7378 | | | | | |
| 34 | G | 902 | 795 | 1272 | 1262 | 0.71 | 0.63 | 1.43 | | | | | | 7378 | | | | | |
| 34 | AAC | 365 | 464 | 1272 | 1262 | 0.29 | 0.37 | 0.69 | | | | | 7378 | 7379 | 7381 | | | | |
| 34 | GAT | 461 | 350 | 1272 | 1262 | 0.36 | 0.28 | 1.48 | | | | | 7378 | 7379 | 7381 | | | | |
| 34 | C | 494 | 593 | 1272 | 1262 | 0.39 | 0.47 | 0.72 | | | | | | 7381 | | | | | |
| 34 | T | 778 | 669 | 1272 | 1262 | 0.61 | 0.53 | 1.40 | | | | | | 7381 | | | | | |
| 34 | CCG | 361 | 431 | 1272 | 1262 | 0.28 | 0.34 | 0.76 | | | | | 7381 | 7382 | 8498 | | | | |
| 34 | CCA | 129 | 161 | 1272 | 1262 | 0.10 | 0.13 | 0.77 | | | | | 7381 | 7382 | 8498 | | | | |
| 34 | TCG | 167 | 116 | 1272 | 1262 | 0.13 | 0.09 | 1.49 | | | | | 7381 | 7382 | 8498 | | | | |
| 34 | CCAAT | 88 | 118 | 1272 | 1262 | 0.07 | 0.09 | 0.72 | | | | 7381 | 7382 | 8498 | 8499 | 8500 | | | |
| 34 | TCGAT | 119 | 72 | 1272 | 1262 | 0.09 | 0.06 | 1.71 | | | | 7381 | 7382 | 8498 | 8499 | 8500 | | | |
| 34 | CCGGC | 43 | 64 | 1272 | 1262 | 0.03 | 0.05 | 0.65 | | | | 7381 | 7382 | 8498 | 8499 | 8500 | | | |
| 34 | TCGGC | 20 | 9 | 1272 | 1262 | 0.02 | 0.01 | 2.22 | | | | 7381 | 7382 | 8498 | 8499 | 8500 | | | |
| 35 | A | 1176 | 1144 | 1262 | 1260 | 0.93 | 0.91 | 1.39 | | | | | | 7437 | | | | | |
| 35 | G | 86 | 116 | 1262 | 1260 | 0.07 | 0.09 | 0.72 | | | | | | 7437 | | | | | |
| 35 | ATT | 1174 | 1142 | 1262 | 1260 | 0.93 | 0.91 | 1.38 | | | | | 7437 | 7439 | 7440 | | | | |
| 35 | GTG | 7 | 19 | 1262 | 1260 | 0.01 | 0.02 | 0.36 | | | | | 7437 | 7439 | 7440 | | | | |
| 35 | T | 1181 | 1150 | 1262 | 1260 | 0.94 | 0.91 | 1.39 | | | | | | 7440 | | | | | |
| 35 | G | 81 | 110 | 1262 | 1260 | 0.06 | 0.09 | 0.72 | | | | | | 7440 | | | | | |
| 35 | TTT | 1066 | 999 | 1262 | 1260 | 0.84 | 0.79 | 1.42 | | | | | 7440 | 7441 | 7442 | | | | |
| 35 | TTC | 111 | 146 | 1262 | 1260 | 0.09 | 0.12 | 0.74 | | | | | 7440 | 7441 | 7442 | | | | |
| 35 | T | 1069 | 1000 | 1262 | 1260 | 0.85 | 0.79 | 1.44 | | | | | | 7442 | | | | | |
| 35 | C | 193 | 260 | 1262 | 1260 | 0.15 | 0.21 | 0.69 | | | | | | 7442 | | | | | |
| 35 | C | 1071 | 1008 | 1262 | 1260 | 0.85 | 0.80 | 1.40 | | | | | | 8501 | | | | | |
| 35 | T | 191 | 252 | 1262 | 1260 | 0.15 | 0.20 | 0.71 | | | | | | 8501 | | | | | |
| 35 | T | 1056 | 995 | 1262 | 1260 | 0.84 | 0.79 | 1.37 | | | | | | 7443 | | | | | |
| 35 | C | 206 | 265 | 1262 | 1260 | 0.16 | 0.21 | 0.73 | | | | | | 7443 | | | | | |
| 36 | AGCAC | 121 | 90 | 1268 | 1262 | 0.10 | 0.07 | 1.37 | | | | 7557 | 8504 | 8505 | 8506 | 8507 | | | |
| 36 | AGCTC | 154 | 190 | 1268 | 1262 | 0.12 | 0.15 | 0.78 | | | | 7557 | 8504 | 8505 | 8506 | 8507 | | | |
| 36 | CAC | 491 | 428 | 1268 | 1262 | 0.39 | 0.34 | 1.23 | | | | | 8505 | 8506 | 8507 | | | | |
| 36 | CTC | 159 | 194 | 1268 | 1262 | 0.13 | 0.15 | 0.79 | | | | | 8505 | 8506 | 8507 | | | | |
| 36 | ACG | 490 | 425 | 1268 | 1262 | 0.39 | 0.34 | 1.24 | | | | | 8506 | 8507 | 7561 | | | | |
| 36 | TCG | 185 | 222 | 1268 | 1262 | 0.15 | 0.18 | 0.80 | | | | | 8506 | 8507 | 7561 | | | | |
| 36 | ACGCT | 294 | 238 | 1268 | 1262 | 0.23 | 0.19 | 1.30 | | | | 8506 | 8507 | 7561 | 8508 | 8509 | | | |
| 36 | TCGCT | 179 | 219 | 1268 | 1262 | 0.14 | 0.17 | 0.78 | | | | 8506 | 8507 | 7561 | 8508 | 8509 | | | |
| 37 | GTC | 248 | 295 | 1264 | 1264 | 0.20 | 0.23 | 0.80 | | | | | 8513 | 8514 | 7613 | | | | |
| 37 | GTG | 851 | 784 | 1264 | 1264 | 0.67 | 0.62 | 1.26 | | | | | 8513 | 8514 | 7613 | | | | |
| 37 | C | 410 | 472 | 1264 | 1264 | 0.32 | 0.37 | 0.81 | | | | | | 7613 | | | | | |
| 37 | G | 854 | 792 | 1264 | 1264 | 0.68 | 0.63 | 1.24 | | | | | | 7613 | | | | | |
| 37 | TCG | 824 | 772 | 1264 | 1264 | 0.65 | 0.61 | 1.19 | | | | | 7619 | 7620 | 7621 | | | | |
| 37 | A | 437 | 488 | 1264 | 1264 | 0.35 | 0.39 | 0.84 | | | | | | 7621 | | | | | |
| 37 | G | 827 | 776 | 1264 | 1264 | 0.65 | 0.61 | 1.19 | | | | | | 7621 | | | | | |
| 40 | GGA | 138 | 105 | 1266 | 1256 | 0.11 | 0.08 | 1.34 | | | | | 7822 | 7823 | 7824 | | | | |
| 40 | GAACC | 257 | 301 | 1266 | 1256 | 0.20 | 0.24 | 0.81 | | | | 7822 | 7823 | 7824 | 7825 | 7826 | | | |
| 40 | AGAAC | 58 | 84 | 1266 | 1256 | 0.05 | 0.07 | 0.67 | | | | 7822 | 7823 | 7824 | 7825 | 7826 | | | |
| 40 | GAAAC | 16 | 2 | 1266 | 1256 | 0.01 | 0.00 | 8.03 | | | | 7822 | 7823 | 7824 | 7825 | 7826 | | | |
| 40 | GGAACCA | 36 | 17 | 1266 | 1256 | 0.03 | 0.01 | 2.13 | | | 7822 | 7823 | 7824 | 7825 | 7826 | 7827 | 7828 | | |
| 40 | GAACCCG | 123 | 166 | 1266 | 1256 | 0.10 | 0.13 | 0.71 | | | 7822 | 7823 | 7824 | 7825 | 7826 | 7827 | 7828 | | |
| 40 | GGACATA | 17 | 6 | 1266 | 1256 | 0.01 | 0.00 | 2.84 | | | 7822 | 7823 | 7824 | 7825 | 7826 | 7827 | 7828 | | |
| 40 | GGAACCACC | 34 | 16 | 1266 | 1256 | 0.03 | 0.01 | 2.14 | | 7822 | 7823 | 7824 | 7825 | 7826 | 7827 | 7828 | 7829 | 7830 | |
| 40 | GAACCCGAT | 123 | 165 | 1266 | 1256 | 0.10 | 0.13 | 0.71 | | 7822 | 7823 | 7824 | 7825 | 7826 | 7827 | 7828 | 7829 | 7830 | |
| 40 | GGACATACC | 17 | 6 | 1266 | 1256 | 0.01 | 0.00 | 2.84 | | 7822 | 7823 | 7824 | 7825 | 7826 | 7827 | 7828 | 7829 | 7830 | |
| 40 | AGAACCACC | 5 | 17 | 1266 | 1256 | 0.00 | 0.01 | 0.29 | | 7822 | 7823 | 7824 | 7825 | 7826 | 7827 | 7828 | 7829 | 7830 | |
| 40 | GAACCCGATTC | 123 | 165 | 1266 | 1256 | 0.10 | 0.13 | 0.71 | 7822 | 7823 | 7824 | 7825 | 7826 | 7827 | 7828 | 7829 | 7830 | 8523 | 8524 |
| 40 | GGACATACCTT | 15 | 3 | 1266 | 1256 | 0.01 | 0.00 | 5.01 | 7822 | 7823 | 7824 | 7825 | 7826 | 7827 | 7828 | 7829 | 7830 | 8523 | 8524 |
| 40 | A | 967 | 891 | 1266 | 1256 | 0.76 | 0.71 | 1.32 | | | | | | 7828 | | | | | |
| 40 | G | 299 | 365 | 1266 | 1256 | 0.24 | 0.29 | 0.75 | | | | | | 7828 | | | | | |
| 40 | C | 971 | 893 | 1266 | 1256 | 0.77 | 0.71 | 1.34 | | | | | | 7829 | | | | | |
| 40 | A | 295 | 363 | 1266 | 1256 | 0.23 | 0.29 | 0.75 | | | | | | 7829 | | | | | |
| 40 | CGATTCC | 163 | 228 | 1266 | 1256 | 0.13 | 0.18 | 0.67 | | | 7827 | 7828 | 7829 | 7830 | 8523 | 8524 | 8526 | | |
| 40 | ATTCC | 167 | 229 | 1266 | 1256 | 0.13 | 0.18 | 0.68 | | | | 7829 | 7830 | 8523 | 8524 | 8526 | | | |
| 40 | TCC | 267 | 333 | 1266 | 1256 | 0.21 | 0.27 | 0.74 | | | | | 8523 | 8524 | 8526 | | | | |
| 40 | T | 873 | 811 | 1266 | 1256 | 0.69 | 0.65 | 1.22 | | | | | | 8526 | | | | | |
| 40 | C | 393 | 445 | 1266 | 1256 | 0.31 | 0.35 | 0.82 | | | | | | 8526 | | | | | |
| 40 | G | 1044 | 964 | 1266 | 1256 | 0.82 | 0.77 | 1.42 | | | | | | 7840 | | | | | |
| 40 | A | 222 | 292 | 1266 | 1256 | 0.18 | 0.23 | 0.70 | | | | | | 7840 | | | | | |
| 42 | CTA | 574 | 517 | 1262 | 1252 | 0.45 | 0.41 | 1.19 | | | | | 7909 | 7910 | 7911 | | | | |
| 42 | TTA | 71 | 102 | 1262 | 1252 | 0.06 | 0.08 | 0.67 | | | | | 7909 | 7910 | 7911 | | | | |
| 42 | CTACA | 125 | 83 | 1262 | 1252 | 0.10 | 0.07 | 1.55 | | | | 7909 | 7910 | 7911 | 7912 | 7913 | | | |
| 42 | TTACG | 61 | 93 | 1262 | 1252 | 0.05 | 0.07 | 0.63 | | | | 7909 | 7910 | 7911 | 7912 | 7913 | | | |
| 42 | CTACAAC | 106 | 78 | 1262 | 1252 | 0.08 | 0.06 | 1.38 | | | 7909 | 7910 | 7911 | 7912 | 7913 | 7915 | 7916 | | |
| 42 | CTACAGC | 13 | 3 | 1262 | 1252 | 0.01 | 0.00 | 4.33 | | | 7909 | 7910 | 7911 | 7912 | 7913 | 7915 | 7916 | | |
| 42 | TTACGGC | 26 | 59 | 1262 | 1252 | 0.02 | 0.05 | 0.43 | | | 7909 | 7910 | 7911 | 7912 | 7913 | 7915 | 7916 | | |
| 42 | CTGCGGT | 9 | 1 | 1262 | 1252 | 0.01 | 0.00 | 8.99 | | | 7909 | 7910 | 7911 | 7912 | 7913 | 7915 | 7916 | | |
| 42 | ACGGC | 155 | 192 | 1262 | 1252 | 0.12 | 0.15 | 0.77 | | | | 7911 | 7912 | 7913 | 7915 | 7916 | | | |
| 42 | ACAAC | 116 | 82 | 1262 | 1252 | 0.09 | 0.07 | 1.44 | | | | 7911 | 7912 | 7913 | 7915 | 7916 | | | |
| 42 | ACAGC | 13 | 4 | 1262 | 1252 | 0.01 | 0.00 | 3.25 | | | | 7911 | 7912 | 7913 | 7915 | 7916 | | | |
| 42 | GCGGT | 11 | 3 | 1262 | 1252 | 0.01 | 0.00 | 3.66 | | | | 7911 | 7912 | 7913 | 7915 | 7916 | | | |
| 42 | G | 1117 | 1146 | 1262 | 1252 | 0.89 | 0.92 | 0.71 | | | | | | 7915 | | | | | |
| 42 | A | 145 | 106 | 1262 | 1252 | 0.11 | 0.08 | 1.40 | | | | | | 7915 | | | | | |
| 42 | ACT | 127 | 92 | 1262 | 1252 | 0.10 | 0.07 | 1.41 | | | | | 7915 | 7916 | 7918 | | | | |
| 43 | T | 553 | 498 | 1262 | 1260 | 0.44 | 0.40 | 1.19 | | | | | | 7956 | | | | | |
| 43 | C | 709 | 762 | 1262 | 1260 | 0.56 | 0.60 | 0.84 | | | | | | 7956 | | | | | |
| 43 | CTCAT | 80 | 113 | 1262 | 1260 | 0.06 | 0.09 | 0.69 | | | | 7956 | 7959 | 7962 | 7963 | 7965 | | | |
| 43 | TCACC | 143 | 180 | 1262 | 1260 | 0.11 | 0.14 | 0.77 | | | | 7959 | 7962 | 7963 | 7965 | 7966 | | | |
| 43 | TCATC | 41 | 70 | 1262 | 1260 | 0.03 | 0.06 | 0.57 | | | | 7959 | 7962 | 7963 | 7965 | 7966 | | | |
| 44 | A | 510 | 575 | 1266 | 1264 | 0.40 | 0.45 | 0.81 | | | | | | 8529 | | | | | |
| 44 | C | 756 | 689 | 1266 | 1264 | 0.60 | 0.55 | 1.24 | | | | | | 8529 | | | | | |
| 44 | TATCCCG | 105 | 139 | 1266 | 1264 | 0.08 | 0.11 | 0.73 | | | 8147 | 8529 | 8530 | 8531 | 8532 | 8533 | 8167 | | |
| 44 | CATTATG | 16 | 45 | 1266 | 1264 | 0.01 | 0.04 | 0.35 | | | 8147 | 8529 | 8530 | 8531 | 8532 | 8533 | 8167 | | |
| 44 | TCCCG | 181 | 227 | 1266 | 1264 | 0.14 | 0.18 | 0.76 | | | | 8530 | 8531 | 8532 | 8533 | 8167 | | | |
| 44 | TTATG | 17 | 45 | 1266 | 1264 | 0.01 | 0.04 | 0.37 | | | | 8530 | 8531 | 8532 | 8533 | 8167 | | | |
| 44 | CCG | 204 | 256 | 1266 | 1264 | 0.16 | 0.20 | 0.76 | | | | | 8532 | 8533 | 8167 | | | | |
| 44 | CCT | 449 | 400 | 1266 | 1264 | 0.35 | 0.32 | 1.19 | | | | | 8532 | 8533 | 8167 | | | | |
| 44 | ATG | 17 | 45 | 1266 | 1264 | 0.01 | 0.04 | 0.37 | | | | | 8532 | 8533 | 8167 | | | | |
| 44 | G | 237 | 327 | 1266 | 1264 | 0.19 | 0.26 | 0.66 | | | | | | 8167 | | | | | |
| 44 | T | 1029 | 937 | 1266 | 1264 | 0.81 | 0.74 | 1.52 | | | | | | 8167 | | | | | |
| 44 | GCT | 121 | 180 | 1266 | 1264 | 0.10 | 0.14 | 0.64 | | | | | 8167 | 8534 | 8535 | | | | |
| 44 | GCTGC | 121 | 179 | 1266 | 1264 | 0.10 | 0.14 | 0.64 | | | | 8167 | 8534 | 8535 | 8536 | 8537 | | | |
| 45 | ACA | 311 | 369 | 1272 | 1264 | 0.24 | 0.29 | 0.78 | | | | | 8538 | 8231 | 8235 | | | | |
| 45 | CAT | 313 | 373 | 1272 | 1264 | 0.25 | 0.30 | 0.78 | | | | | 8231 | 8235 | 8236 | | | | |
| 45 | TAT | 94 | 67 | 1272 | 1264 | 0.07 | 0.05 | 1.43 | | | | | 8231 | 8235 | 8236 | | | | |
| 45 | ATC | 315 | 371 | 1272 | 1264 | 0.25 | 0.29 | 0.79 | | | | | 8235 | 8236 | 8539 | | | | |
| 45 | C | 378 | 424 | 1272 | 1264 | 0.30 | 0.34 | 0.84 | | | | | | 8539 | | | | | |
| 45 | T | 894 | 840 | 1272 | 1264 | 0.70 | 0.66 | 1.19 | | | | | | 8539 | | | | | |
| 45 | A | 286 | 343 | 1272 | 1264 | 0.22 | 0.27 | 0.78 | | | | | | 8239 | | | | | |
| 45 | G | 986 | 921 | 1272 | 1264 | 0.78 | 0.73 | 1.28 | | | | | | 8239 | | | | | |
| 45 | G | 286 | 343 | 1272 | 1264 | 0.22 | 0.27 | 0.78 | | | | | | 8241 | | | | | |
| 45 | A | 986 | 921 | 1272 | 1264 | 0.78 | 0.73 | 1.28 | | | | | | 8241 | | | | | |
| 47 | CAG | 31 | 16 | 1124 | 1130 | 0.03 | 0.01 | 1.97 | | | | | 8542 | 8543 | 8336 | | | | |
| 47 | CAGTG | 17 | 5 | 1124 | 1130 | 0.02 | 0.00 | 3.46 | | | | 8542 | 8543 | 8336 | 8338 | 8340 | | | |
| 47 | C | 332 | 386 | 1124 | 1130 | 0.30 | 0.34 | 0.81 | | | | | | 8350 | | | | | |
| 47 | T | 792 | 744 | 1124 | 1130 | 0.70 | 0.66 | 1.24 | | | | | | 8350 | | | | | |
| 47 | C | 330 | 385 | 1124 | 1130 | 0.29 | 0.34 | 0.80 | | | | | | 8544 | | | | | |
| 47 | T | 794 | 745 | 1124 | 1130 | 0.71 | 0.66 | 1.24 | | | | | | 8544 | | | | | |
| 47 | A | 330 | 383 | 1124 | 1130 | 0.29 | 0.34 | 0.81 | | | | | | 8545 | | | | | |
| 47 | G | 794 | 747 | 1124 | 1130 | 0.71 | 0.66 | 1.23 | | | | | | 8545 | | | | | |

1. A method of constructing a longevity trait GeneMap in a human population, comprising screening for the expression level of or presence or absence of at least one allele of (i) at least one gene from Tables 4, 5 and 6 or (ii) at least one single nucleotide polymorphism (SNP) from Tables 2, 3 and 7 in at least one sample.

2. A method of claim 1, wherein said population is a general population.

3. A method of claim 1, wherein said population is a founder population.

4. A method of claim 3, wherein said founder population is the population of Quebec.

5-21. (canceled)

22. A set of genetic markers comprising at least two SNPs of Tables 2, 3 and 7.

23. A set of nucleic acid probes that specifically detect said SNPs of claim 22.

24. A solid support or collection of solid supports comprising the nucleic acid probes of claim 23.

25. A solid support of claim 24, wherein the support is selected from the group consisting of at least one microarray and a set of beads.

26. A method for predicting the efficacy of a drug for treating an age-associated disorder in a human patient, comprising: a) obtaining a sample of cells from the patient; b) obtaining (i) a gene expression profile or (ii) a set of genotype from the sample in the absence and presence of in vitro modulation of the cells with specific mediators; the gene expression profile comprising one or more genes from Tables 4, 5 and 6; the set of genotypes comprising one or more polymorphic loci from Tables 2, 3 and 7 and c) comparing the gene expression profile or the set of genotypes of the sample with a reference gene expression profile or a reference set of genotypes associated with efficacy of the drug, wherein similarity between the sample expression profile and the reference expression profile or between the set of genotypes and the reference set of genotypes predicts the efficacy of the drug for treating age-associated disorder in the patient.

27. The method of claim 26, further comprising exposing the sample to the drug for treating an age-associated disorder prior to obtaining the gene expression profile of the sample.

28. The method of claim 26, wherein the sample of cells is derived from a tissue selected from the group consisting of: any parts of the body such as the scalp, blood, dermis, epidermis and other skin cells, cutaneous surfaces, intertrigious areas, genitalia, vessels and endothelium.

29. The method of claim 28, wherein the cells are selected from the group consisting of: red blood cells, muscle cells, heart cells, nerve cells, insulin-producing cells, pancreatic cells, brain cells, germ cells, keratinocytes, monocytes, neutrophils, Langerhans cells, CD4+ and CD8+ T cells, B and T lymphocytes, leukocytes, hormonal cells, bone marrow cells, skin cells, buccal cells, spinal cord cells, bone cells, adipose cells, cartilage cells, dendritic cells, intestinal cells, hepatic cells, mucous cells, olfactory cells, retinal cells, somatic cells and arterial cells.

30. The method of claim 26, wherein the sample is obtained via biopsy.

31. The method of claim 26, wherein the gene expression profile comprises expression values for at least two of the genes listed in Tables 4, 5 and 6.

32. The method of claim 31, wherein the gene expression profile of the sample is obtained by detecting the protein products of said genes.

33. The method of claim 26, wherein the gene expression profile of the sample is obtained using a hybridization assay to oligonucleotides contained in a microarray.

34. The method of claim 26, wherein the oligonucleotides comprises nucleic acid molecules at least 95% identical to SEQ ID from Tables 2, 3, 4, 5 or 7.

35. The method of claim 26, wherein the reference expression profile is that of cells derived from patients that have the longevity trait.

36. The method of claim 26, wherein the drug is selected from the group consisting of symptom relievers and drugs for an age-associated condition.

37. A method for inducing a longevity-like state in a resident tissue or cell, comprising contacting the tissue or cell with at least one gene from Tables 4, 5 and 6 that induces a longevity-like state.

38. The method of claim 37, wherein the resident tissue cell is selected from the group consisting of red blood cells, muscle cells, heart cells, nerve cells, insulin-producing cells, pancreatic cells, brain cells, germ cells, keratinocytes, monocytes, neutrophils, Langerhans cells, CD4+ and CD8+ T cells, B and T lymphocytes, leukocytes, hormonal cells, bone marrow cells, skin cells, buccal cells, spinal cord cells, bone cells, adipose cells, cartilage cells, dendritic cells, intestinal cells, hepatic cells, mucous cells, olfactory cells, retinal cells, somatic cells and arterial cells.

39. A method for screening drug candidates for treating age-associated disorders, comprising: a) contacting a resident cell induced by the method of claim 37 with a drug candidate for treating age-associated disorders; and b) assaying for a longevity-like state, such that an absence of the longevity-like state is indicative of the drug candidate being effective in treating age-associate disorders.

40-43. (canceled)

44. A drug screening assay comprising: a) administering a test compound to an animal having an age-associated disorder, or related phenotype, or a cell composition isolated therefrom; and b) comparing the level of gene expression of at least one gene from Tables 4, 5 and 6 in the presence of the test compound with one or both of the level of said gene expression in the absence of the test compound or in normal cells; wherein test compounds which cause the level of expression of one or more genes from Tables 4, 5 and 6 to approach normal are candidates for drugs to treat age-associated disorders.

45-46. (canceled)

47. A method for identifying a gene that regulates drug response in an age-associated disorder, comprising: a) obtaining a gene expression profile for at least one gene from Tables 4, 5 and 6 in a resident tissue cell induced for a longevity-like state in the presence of the candidate drug; and b) comparing the expression profile of said gene to a reference expression profile for said gene in a cell induced for the longevity-like state in the absence of the candidate drug, wherein genes whose expression relative to the reference expression profile is altered by the drug may identifies the gene as a gene that regulates drug response in the longevity trait.

48. An expression profile indicative of the presence of longevity trait in a patient, comprising the level of expression of at least one gene of Tables 4, 5 and 6.

49. A microarray comprising probes that hybridize to one or more genes of Tables 4, 5 and 6.

50-51. (canceled)

52. A method of diagnosing susceptibility to an age-associated disorder in an individual, comprising screening for an at-risk haplotype of at least one gene from Tables 4, 5 and 6, or comprising at least one SNP from Tables 2, 3 and 7, that is more frequently present in an individual susceptible to an age-associated disorder, compared to the frequency of its presence in a control individual, wherein the presence of the at-risk haplotype is indicative of a susceptibility to an age-associated disorder.

53. The method of claim 52, wherein the at-risk haplotype is characterized by the presence of at least one single nucleotide polymorphism from Tables 2, 3 and 7.

54. The method of claim 52, wherein screening for the presence of an at-risk haplotype in at least one gene from Tables 4, 5 and 6, or comprising at least one SNP from Tables 2, 3 and 7, comprises enzymatic amplification of nucleic acid from said individual or amplification using universal oligos on elongation/ligation products.

55. The method of claim 54, wherein the nucleic acid is DNA.

56. The method of claim 55, wherein the DNA is human DNA.

57. The method of claim 52, wherein screening for the presence of an at-risk haplotype in at least one gene from Tables 4, 5 and 6 or comprising at least one SNP from Tables 2, 3 and 7 comprises: a) obtaining material containing nucleic acid from the individual; (b) amplifying said nucleic acid as for claim 54; and c) determining the presence or absence of an at-risk haplotype in said amplified nucleic acid.

58. The method of claim 57, wherein determining the presence of an at-risk haplotype is performed by electrophoretic analysis, restriction length polymorphism analysis, sequence analysis or hybridization analysis.

59-95. (canceled)

96. A method for determining the phenotype of a cell comprising detecting the differential expression, relative to a normal cell, of at least one gene from Tables 4, 5 and 6.

97. The method of claim 96, wherein said difference in the level of expression of said gene, is of at least a factor of about two.

98. The method of claim 96, including the further step of cloning said genes which are up- or down-regulated.

99. The method of claim 96, including the further step of generating nucleic acid probes for detecting the level of expression of said genes which are up- or down-regulated.

100. A kit for assessing a patient's risk of having or developing an age-associated disorder, comprising: a) detection means for detecting the differential expression, relative to a normal cell, of at least one gene shown in Tables 4, 5 and 6 or the gene product thereof; and b) instructions for correlating the differential expression of said gene or gene product with a patient's risk of having or developing an age-associated disorder.

101. The kit of claim 100, wherein the detection means includes nucleic acid probes for detecting the level of mRNA of said genes.

102. A kit for assessing a patient's risk of having or developing an age-associated disorder, comprising: (a) at least one means for amplifying or detecting a sequence of at least one gene in Tables 4, 5 and 6, or at least one sequence comprising a SNP in Tables 2, 3 and 7, wherein the detection means includes nucleic acid probes or primers for detecting the presence or absence of an associated allele, a particular allele of a polymorphic locus, or the like or changes to at least one sequence of Tables 4, 5 and 6 or Tables 2, 3 and 7, and (b) instructions for correlating the presence or absence of at least one sequence of Tables 4, 5 and 6 or Tables 2, 3 and 7 with a patient's risk of having age-associated disorder.

103. The kit of claim 102, wherein the detection means includes an immunoassay for detecting the level of at least one gene product from Tables 4, 5 and 6.

104. A method of assessing a patient's risk of having or developing an age-associated disorder, comprising: a) determining the level of expression of at least one gene from Tables 4, 5 and 6 or gene products thereof, and comparing the level of expression to a normal cell; and b) assessing a patient's risk of having or developing an age-associated disorder, if any, by determining the correlation between the differential expression of said genes or gene products with known changes in expression of said genes measured in at least one parent suffering from an age-associated disorder.

105. A nucleic acid array comprising a solid support comprising nucleic acid probes which selectively hybridize to at least 5 different genes from Tables 4, 5 and 6 or at least 5 different SNPs of Tables 2, 3 and 7.

106. The array of claim 105, wherein the solid support is selected from the group consisting of paper, membranes, filters, chips, pins, and glass.

107. A method of diagnosing the longevity trait in a patient, comprising detecting a nucleic acid molecule encoding at least one protein from Tables 4, 5 and 6 in a fluid or tissue sample from the patient.

108. A method of claim 107, wherein the detecting comprises detecting at least one associated allele, particular allele of a polymorphic locus, or the like in the nucleic acid molecule encoding said protein.

109. A method of claim 107, wherein said method comprises hybridizing a probe to said patient's sample of DNA or RNA under stringent conditions which allow hybridization of said probe to nucleic acid comprising said associated allele, a particular allele of a polymorphic locus, or the like, wherein the presence of a hybridization signal indicates the presence of said associated allele, particular allele of a polymorphic locus, or the like, in at least one gene from Tables 4, 5 and 6.

110. A method of claim 109, wherein the patient's DNA or RNA has been amplified and said amplified DNA or RNA is hybridized.

111. A method of claim 109, wherein said method comprises using a single-stranded conformation polymorphism technique to assay for said associated allele, particular allele of a polymorphic locus, or the like.

112. (canceled)

113. A method of claim 109, wherein said patient's sample of DNA has been amplified or cloned.

114. A method of claim 109, wherein said method comprises sequencing at least one gene from Tables 4, 5 and 6 in a sample of RNA or DNA from a patient.

115. A method of claim 109, wherein said method comprises determining the sequence of at least one gene from Tables 4, 5 and 6 by preparing cDNA from RNA taken from said patient and sequencing said cDNA to determine the presence or absence of an associated allele, a particular allele of a polymorphic locus, or the like.

116. A method of claim 109, wherein said method comprises performing an RNAse assay.

117. A method of claim 109, wherein said probe is attached to a microarray or a bead.

118. A method of claim 109, wherein said probes are oligonucleotides.

119. A method of claim 109, wherein said sample is selected from the group consisting of blood, normal tissue and tumor tissue.

120. A method of claim 109, wherein the associated allele, particular allele of a polymorphic locus, or the like is selected from the group consisting of at least one of the SNPs from Tables 2, 3 and 7, alone or in combination.

121-123. (canceled)

124. A method for diagnosing or prognosticating longevity comprising comparing the level of expression or activity of a polypeptide encoded by a gene of Tables 4, 5 and 6 in a test sample from a patient with the level of expression or activity of the same polypeptide in a control sample wherein a difference in the level of expression or activity between the test sample and control sample is indicative of the longevity trait.

125-131. (canceled)

132. The method of claim 26, wherein the set of genotypes from the sample comprises genotypes of at least two of the polymorphic loci listed in Tables 2, 3 and 7.

133. The method of claim 26, wherein the set of genotypes from the sample is obtained by hybridization to allele-specific oligonucleotides complementary to the polymorphic loci from Tables 2, 3 and 7, wherein said allele-specific oligonucleotides are contained on a microarray.

134. The method of claim 133, wherein the oligonucleotides comprise nucleic acid molecules at least 95% identical to SEQ ID from Tables 1, 2 or 3.

135. The method of claim 26, wherein the set of genotypes from the sample is obtained by sequencing said polymorphic loci in said sample.

136. The method of claim 26, wherein the drug is selected from the group consisting of symptom relievers and drugs for an age-associated condition.

137-145. (canceled)

146. A kit for assessing a patient's risk of having or developing an age-associated disorder, comprising: a) detection means for detecting the genotype of at least one polymorphic locus shown in Tables 2, 3 and 7; and b) instructions for correlating the genotype of said at least one polymorphic locus with a patient's risk of having or developing an age-associated disorder.

147. The kit of claim 146, wherein the detection means includes nucleic acid probes for detecting the genotype of said at least one polymorphic locus.

148. A method of assessing a patient's risk of having or developing an age-associated disorder, comprising: a) selecting at least one polymorphic locus from Tables 2, 3 and 7; b) determining a genotype for said at least one polymorphic locus from Table 1 in a patient; c) comparing said genotype of b) to a genotype for said at least one polymorphic locus from Tables 2, 3 and 7 that is associated with the age-associated disorder; and d) assessing the patient's risk of having or developing the age-associated disorder, wherein said patient has a higher risk of having or developing the age-associated disorder if the genotype for said at least one polymorphic locus from Tables 2, 3 and 7 in said patient is the same as said genotype for said at least one polymorphic locus from Tables 2, 3 and 7 that is associated with the age-associated disorder.
